(19) United States
(12) Patent Application Publication
(10) Pub. No.: US 2023/0322744 A1
(43) Pub. Date: Oct. 12, 2023

(54) COMPOUNDS AS GLP-1R AGONISTS

(71) Applicant: Terns Pharmaceuticals, Inc., Foster City, CA (US)

(72) Inventors: F. Anthony ROMERO, Redwood City, CA (US); Christopher T. JONES, Foster City, CA (US); Martijn FENAUX, San Mateo, CA (US); Corey REEVES, Foster City, CA (US); Thorsten A. KIRSCHBERG, San Carlos, CA (US); Yingzi XU, Palo Alto, CA (US)

(21) Appl. No.: 18/042,443

(22) PCT Filed: Aug. 20, 2021
(86) PCT No.: PCT/US2021/047015

§ 371 Date: Feb. 21, 2023

Related U.S. Application Data
(60) Provisional application No. 63/068,870, filed on Aug. 21, 2020.

(51) Int. Cl. C07D 405/14; C07D 487/04; C07D 498/04; C07D 417/14; C07D 471/04; C07D 409/14

(52) U.S. Cl. CPC C07D 405/14; C07D 487/04; C07D 498/04; C07D 417/14; C07D 471/04; C07D 409/14

(57) ABSTRACT

The present application provides compounds that may be used as a glucagon-like peptide-1 receptors (GLP-1R) agonist, or stereoisomers, tautomers, or pharmaceutically acceptable salts of any of the foregoing. Also provided are pharmaceutical compositions containing such compounds, or stereoisomers, tautomers, or pharmaceutically acceptable salts of any of the foregoing. Methods of prepare these compounds and compositions and method of using them to treat or present a disease or a condition mediated by GLP-1R.

### CROSS-REFERENCE TO RELATED APPLICATIONS

This application claims priority to U.S. Provisional Patent Application No. 63/068,870, filed Aug. 21, 2020, the disclosure of which is hereby incorporated herein by reference in its entirety for all purposes.

### FIELD

This invention relates to compositions for modulating glucagon-like peptide-1 (GLP-1) receptors and methods thereof.

### BACKGROUND

Diabetes is a major public health concern because of its increasing prevalence and associated health risks. The disease is characterized by high levels of blood glucose resulting from defects in insulin production, insulin action, or both. Two major forms of diabetes are recognized, Type 1 and Type 2. Type 1 diabetes (T1D) develops when the body's immune system destroys pancreatic beta cells, the only cells in the body that make the hormone insulin that regulates blood glucose. To survive, people with Type 1 diabetes must have insulin administered by injection or a pump. Type 2 diabetes mellitus (T2DM) usually begins with either insulin resistance or when there is insufficient production of insulin to maintain an acceptable glucose level.

Currently, various pharmacological approaches are available for treating hyperglycemia and subsequently, T2DM (Hampp, C. et al. Use of Antidiabetic Drugs in the U.S., 2003-2012, Diabetes Care 2014, 37, 1367-1374). One of them is glucagon-like peptide-1 receptor (GLP-1R) agonists (e.g., liraglutide, albiglutide, exenatide, lixisenatide, dulaglutide, semaglutide), which enhance secretion of insulin by acting on the pancreatic beta-cells. Marketed GLP-1R agonists are peptides administered by subcutaneous injection. Liraglutide is additionally approved for the treatment of obesity.

GLP-1 is a 30 amino acid long incretin hormone secreted by the L-cells in the intestine in response to ingestion of food. GLP-1 has been shown to stimulate insulin secretion in a physiological and glucose-dependent manner, decrease glucagon secretion, inhibit gastric emptying, decrease appetite, and stimulate proliferation of beta-cells. In non-clinical experiments GLP-1 promotes continued beta-cell competence by stimulating transcription of genes important for glucose-dependent insulin secretion and by promoting beta-cell neogenesis (Meier et al. Biodrugs. 2003; 17 (2): 93-102).

In a healthy individual, GLP-1 plays an important role regulating post-prandial blood glucose levels by stimulating glucose-dependent insulin secretion by the pancreas resulting in increased glucose absorption in the periphery. GLP-1 also suppresses glucagon secretion, leading to reduced hepatic glucose output. In addition, GLP-1 delays gastric emptying and slows small bowel motility delaying food absorption. In people with T2DM, the normal post-prandial rise in GLP-1 is absent or reduced (Vilsboll T, et al. Diabetes. 2001. 50; 609-613).

Holst (Physiol. Rev. 2007, 87, 1409) and Meier (Nat. Rev. Endocrinol. 2012, 8, 728) describe that GLP-1 receptor agonists, such as liraglutide and exendin-4, have 3 major pharmacological activities to improve glycemic control in patients with T2DM by reducing fasting and postprandial glucose (FPG and PPG): (i) increased glucose-dependent insulin secretion (improved first- and second-phase), (ii) glucagon suppressing activity under hyperglycemic conditions, (iii) delay of gastric emptying rate resulting in retarded absorption of meal-derived glucose.

There remains a need of developing GLP-1 receptor agonists for an easily-administered prevention and/or treatment for cardiometabolic and associated diseases.

### BRIEF SUMMARY

Disclosed are compounds that can be used as glucagon-like peptide-1 receptor (GLP-1R) agonists, compositions containing these compounds and methods for treating diseases and/or conditions mediated by GLP-1R.

In one aspect, provides is a compound of Formula (I), including compounds of Formulae (II)-(IV), or selected from the group consisting of a compound listed in Table 1, or a stereoisomer, tautomer, or a pharmaceutically acceptable salt of any of the foregoing, as detailed herein.

Further provided is a pharmaceutical composition comprising is a compound of Formula (I), including compounds of Formulae (II)-(IV), or selected from the group consisting of a compound listed in Table 1, or a stereoisomer, tautomer, or a pharmaceutically acceptable salt of any of the foregoing, and a pharmaceutically acceptable carrier or excipient.

In another aspect, provided is a method of treating a disease or a condition mediated by GLP-1R in a subject in need thereof comprises administering to the subject a therapeutically effective amount of a compound of Formula (I), including compounds of Formulae (II)-(IV), or selected from the group consisting of compounds listed in Table 1, or a stereoisomer, tautomer, or a pharmaceutically acceptable salt of any of the foregoing. In some embodiments, the disease or the condition is a cardiometabolic disease. In some embodiments, the disease or the condition is diabetes. In some embodiments, the disease or the condition is a liver disease.

Also provided is a compound of Formula (I), including compounds of Formulae (II)-(IV), or selected from the group consisting of a compound listed in Table 1, or a stereoisomer, tautomer, or a pharmaceutically acceptable salt of any of the foregoing, as detailed herein, for the treatment.

Also provided is use of a compound of Formula (I), including compounds of Formulae (II)-(IV), or selected from the group consisting of a compound listed in Table 1, or a stereoisomer, tautomer, or a pharmaceutically acceptable salt of any of the foregoing, as detailed herein, in the manufacture of a medicament for the treatment.

Further provided is a kit comprising a compound of Formula (I), including compounds of Formulae (II)-(IV), or selected from the group consisting of a compound listed in Table 1, or a stereoisomer, tautomer, or a pharmaceutically acceptable salt of any of the foregoing. In some embodiments, the kit comprises instructions for use according to a method described herein.

In yet another aspect, provided is a method of making a compound of Formula (I), including compounds of Formulae (II)-(IV), or selected from the group consisting of a compound listed in Table 1, or a stereoisomer, tautomer, or a pharmaceutically acceptable salt of any of the foregoing. Also provided are compound intermediates useful in synthesis of a compound of Formula (I), including compounds of Formulae (II)-(IV), or selected from the group consisting of a compound listed in Table 1, or a stereoisomer, tautomer, or a pharmaceutically acceptable salt of any of the foregoing.

### DETAILED DESCRIPTION

### Definitions

As used herein, the following definitions shall apply unless otherwise indicated. Further, if any term or symbol used herein is not defined as set forth below, it shall have its ordinary meaning in the art.

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in connection with a value, contemplate a variation within ±15%, within ±10%, within ±5%, within ±4%, within ±3%, within ±2%, within ±1%, or within +0.5% of the specified value. Reference to “about” a value or parameter herein includes (and describes) embodiments that are directed to that value or parameter per se. For example, description referring to “about X” includes description of “X”.

“Comprising” is intended to mean that the compositions and methods include the recited elements, but not exclude others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH4, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the likes.

“Stereoisomer” or “stereoisomers” refer to compounds that differ in the stereogenicity of the constituent atoms such as, without limitation, in the chirality of one or more stereocenters or related to the cis or trans configuration of a carbon-carbon or carbon-nitrogen double bond. Stereoisomers include enantiomers and diastereomers.

As used herein, the term “subject” refers to an animal, including, but are not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

As used herein, “treatment” or “treating” is an approach for obtaining beneficial or desired results including clinical results. For purposes of this disclosure, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of this disclosure contemplate any one or more of these aspects of treatment.

“Therapeutically effective amount” or dose of a compound or a composition refers to that amount of the compound or the composition that results in reduction or inhibition of symptoms or a prolongation of survival in a patient. The results may require multiple doses of the compound or the composition.

“Alkyl” refers to monovalent saturated aliphatic hydrocarbyl groups having from 1 to 12 carbon atoms, preferably from 1 to 10 carbon atoms, and more preferably from 1 to 6 carbon atoms. This term includes, by way of example, linear and branched hydrocarbyl groups such as methyl (CH3-), ethyl (CH3CH2-), n-propyl (CH3CH2CH2-), isopropyl ((CH3)2CH—), n-butyl (CH3CH2CH2CH2-), isobutyl ((CH3)2CHCH2-), sec-butyl ((CH3)(CH3CH2)CH—), t-butyl ((CH3)3C—), n-pentyl (CH3CH2CH2CH2CH2-), and neopentyl ((CH3)3CCH2-). Cx alkyl refers to an alkyl group having x number of carbon atoms.

“Alkylene” refers to a divalent saturated aliphatic hydrocarbyl group having from 1 to 12 carbon atoms, preferably from 1 to 10 carbon atoms, and more preferably from 1 to 6 carbon atoms. This term includes, by way of example, linear and branched hydrocarbyl groups such as methylene (—CH2-), ethylene (—CH2CH2- or —CH(Me)-), propylene (—CH2CH2CH2- or —CH(Me)CH2-, or —CH(Et)-) and the likes.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Aryl” refers to a monovalent aromatic carbocyclic group of from 6 to 14 carbon atoms having a single ring (e.g., phenyl (Ph)) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Cyano” refers to the group —C≡N.

“Cycloalkyl” refers to saturated or unsaturated but nonaromatic cyclic alkyl groups of from 3 to 10 carbon atoms, preferably from 3 to 8 carbon atoms, and more preferably from 3 to 6 carbon atoms, having single or multiple cyclic rings including fused, bridged, and spiro ring systems. Cx cycloalkyl refers to a cycloalkyl group having x number of ring carbon atoms. Examples of suitable cycloalkyl groups include, for instance, adamantyl, cyclopropyl, cyclobutyl, cyclopentyl, and cyclooctyl. One or more the rings can be aryl, heteroaryl, or heterocyclic provided that the point of attachment is through the non-aromatic, non-heterocyclic ring saturated carbocyclic ring. “Substituted cycloalkyl” refers to a cycloalkyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, S03H, substituted sulfonyl, sulfonyloxy, thioacyl, thiol, alkylthio, and substituted alkylthio, wherein said substituents are defined herein.

“Halo” or “halogen” refers to fluoro, chloro, bromo and iodo and preferably is fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include 5 or 6 membered heteroaryls such as pyridinyl, pyrrolyl, thiophenyl, and furanyl. Other preferred heteroaryls include 9 or 10 membered heteroaryls, such as indolyl, quinolinyl, quinolonyl, isoquinolinyl, and isoquinolonyl.

“Heterocycle” or “heterocyclic” or “heterocycloalkyl” or “heterocyclyl” refers to a saturated or partially saturated, but not aromatic, group having from 1 to 10 ring carbon atoms, preferably from 1 to 8 carbon atoms, and more preferably from 1 to 6 carbon atoms, and from 1 to 4 ring heteroatoms, preferably from 1 to 3 heteroatoms, and more preferably from 1 to 2 heteroatoms selected from the group consisting of nitrogen, sulfur, or oxygen. Cx heterocycloalkyl refers to a heterocycloalkyl group having x number of ring atoms including the ring heteroatoms. Heterocycle encompasses single ring or multiple condensed rings, including fused bridged and spiro ring systems. In fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl (S(O)), sulfonyl (S(O)2) moieties.

Examples of heterocyclyl and heteroaryl include, but are not limited to, azetidinyl, pyrrolyl, imidazolyl, pyrazolyl, pyridyl, pyrazyl, pyrimidyl, pyridazyl, indolizyl, isoindolyl, indolyl, dihydroindolyl, indazolyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholinyl, thiomorpholinyl (also referred to as thiamorpholinyl), 1,1-dioxothiomorpholinyl, piperidinyl, pyrrolidinyl, and tetrahydrofuranyl.

“Oxo” refers to the atom (═O) or (O).

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

It is understood that an optionally substituted moiety can be substituted with more than five substituents, if permitted by the number of valences available for substitution on the moiety. For example, a propyl group can be substituted with seven halogen atoms to provide a perhalopropyl group. The substituents may be the same or different.

### Compounds

In one aspect, provided is a compound of Formula (I):

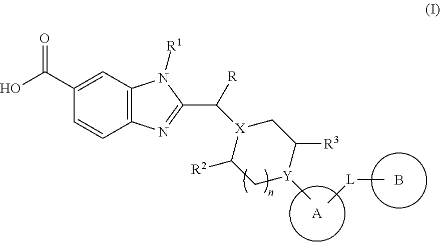

or a stereoisomer, tautomer, or a pharmaceutically acceptable salt of any of the foregoing, wherein:
X is N or CH;
Y is N or CR⁴, wherein R⁴ is hydrogen, OH or C₁-C₆ alkyl;
n is 0 or 1;
R is hydrogen;
R¹ is —C₁-C₆ alkylene-R⁵, wherein R⁵ is 3- to 6-membered heterocyclyl or 5- to 6-membered heteroaryl, each of which is independently optionally substituted by C₁-C₆ alkyl, or
   R¹ is taken together with R and the intervening atoms to form a Ring C, wherein Ring C is a 5- to 7-membered heterocyclyl optionally substituted by C₁-C₆ alkyl;
R² and R³ are independently hydrogen, oxo, or C₁-C₆ alkyl, wherein when Y is CR⁴, R³ and
R⁴ are optionally taken together with the carbon atoms to which they are attached to form C₃-C₆ cycloalkyl;
Ring A is 5- to 12-membered heterocyclyl or 5- to 12-membered heteroaryl, each of which is independently optionally substituted by halo, CN, C₃-C₆ cycloalkyl, or C₁-C₆ alkyl optionally substituted by halo or OH;
L is a bond, —O—, C₁-C₆ alkylene, *—O—C₁-C₆ alkylene-**, *—C₁-C₆ alkylene-O—**, or *—NR⁶—C₁-C₆ alkylene-**, wherein
   * represents the point of attachment to ring A and ** represents the point of attachment to ring B,
   when L is *—O—C₁-C₆ alkylene-**, the C₁-C₆ alkylene is optionally substituted by R^{L}, wherein:
   each R^{L} is independently C₁-C₆ alkyl or halo, or two R^{L} are taken together with the carbon atom or atoms to which they are attached to form C₃-C₆ cycloalkyl or 3- to 6-membered heterocyclyl,
   when L is C₁-C₆ alkylene, the C₁-C₆ alkylene is optionally substituted by R^{L1}, wherein:
      each R^{L1} is independently halo, OH, or C₁-C₆ alkyl; or two R^{L1} are taken together with the carbon atom or atoms to which they are attached to form C₃-C₆ cycloalkyl or 3- to 6-membered heterocyclyl; and
   R⁶ is hydrogen or C₁-C₆ alkyl; and
Ring B is C₃-C₁₀ cycloalkyl, C₆-C₁₄ aryl, 4- to 12-membered heterocyclyl, or 5- to 12-membered heteroaryl, each of which is independently optionally substituted by one to three substituents independently selected from the group consisting of halo, CN, oxo, C₁-C₆ alkyl, C₁-C₆ haloalkyl, —COCH₃, —CONH₂, —S(O)₂CH3, and phenyl, with the proviso that
   when R¹ is —C₁-C₆ alkylene-R⁵, wherein R⁵ is 3- to 6-membered heterocyclyl or 3- to 6-membered heteroaryl, each of which is optionally substituted by C₁-C₆ alkyl, Y is N or CH, n is 1, R² and R³ are independently hydrogen or C₁-C₆ alkyl, ring A is 6-membered heteroaryl optionally substituted one or two substituents each independently selected from the group consisting of F, Cl and CN, and L is *—OCH₂—**, then ring B is not phenyl optionally substituted by one or two substituents each independently selected from the group consisting of halo, CN, and C₁-C₆ alkyl;
   when R¹ is —C₁-C₆ alkylene-R⁵, wherein R⁵ is 3- to 6-membered heterocyclyl or 3- to 6-membered heteroaryl, each of which is optionally substituted by C₁-C₆ alkyl, Y is N or CH,
n is 1, R² and R³ are independently hydrogen or C₁-C₆ alkyl, ring A is

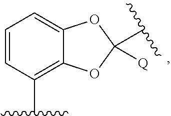

wherein Q is H or CH3, and L is a bond, then ring B is neither phenyl or pyridinyl, each of which is optionally substituted by one or two substituents each independently selected from the group consisting of halo, CN, and C₁-C₆ alkyl; and
   when R¹ is —C₁-C₆ alkylene-R⁵, wherein R⁵ is 4-membered heterocyclyl or 5-membered heteroaryl, each of which is optionally substituted by C₁-C₆ alkyl, X is N, Y is N or CH, n is 1, and R² and R³ are independently hydrogen or oxo, then ring B is not

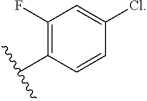 [SMILES: *c1ccc(Cl)cc1F]

In some such embodiments of Formula (I), Ring B is C₃-C₁₀ cycloalkyl, C₆-C₁₄ aryl, 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH3, and phenyl. In some such embodiments of Formula (I), including formulae (II), (V), (Va), (Vb), (Vb-1), (I′), (I″), and (VI), when L is *—O—C₁-C₆ alkylene-**, the C₁-C₆ alkylene is optionally substituted by R^{L}, wherein each R^{L} is independently C₁-C₆ alkyl, or two R^{L} are taken together with the carbon atom or atoms to which they are attached to form C₃-C₆ cycloalkyl or 3- to 6-membered heterocyclyl. In some such embodiments of Formula (I), when L is C₁-C₆ alkylene, the C₁-C₆ alkylene is unsubstituted.

In the descriptions herein, it is understood that every description, variation, embodiment or aspect of a moiety/variable may be combined with every description, variation, embodiment or aspect of other moieties/variables the same as if each and every combination of descriptions is specifically and individually listed. For example, every description, variation, embodiment or aspect provided herein with respect to R¹ of Formula (I) may be combined with every description, variation, embodiment or aspect of Ring A the same as if each and every combination were specifically and individually listed.

It is also understood that the provisos provided herein may apply to each embodiment of compounds of Formulae (I)-(IV) described herein as long as any of them are applicable.

In some embodiments, provided is a compound of Formula (II):

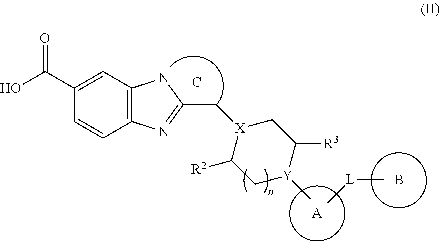

or a stereoisomer, tautomer, or a pharmaceutically acceptable salt of any of the foregoing, wherein Ring C is a 5- to 7-membered heterocyclyl optionally substituted by C₁-C₆ alkyl, and X, Y, n, R², R³, Ring A, Ring B and L are as detailed herein for Formula (I).

In some embodiments, provided is a compound of Formula (III):

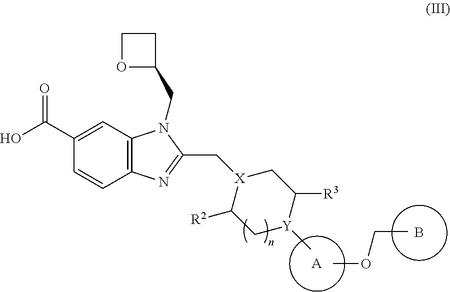

or a stereoisomer, tautomer, or a pharmaceutically acceptable salt of any of the foregoing, wherein X, Y, n, R², R³, Ring A, and Ring B are as detailed herein for Formula (I) with the provisos if applicable. In some embodiments, X is N and Y is CR⁴. In some embodiments, X is CH and Y is N. In some embodiments, X and Y are each N.

In some embodiments of Formula (III), X is N, Y is CR⁴; R³ and R⁴ are taken together with the carbon atoms to which they are attached to form a C₃-C₆ cycloalkyl; and n, R², Ring A, and Ring B are as detailed herein for Formula (I). In some embodiments of Formula (III), Y is CR⁴; R³ and R⁴ are taken together with the carbon atoms to which they are attached to form a C₃-C₆ cycloalkyl; Ring B is optionally substituted phenyl; and X, n, R², and Ring A are as detailed herein for Formula (I). In some embodiments of the foregoing, R³ and R⁴ are taken together with the carbon atoms to which they are attached to form a C₃ cycloalkyl. In some embodiments of the foregoing, X is N. In some embodiments of the foregoing, n is 1. In some embodiments of the foregoing, R² is H. In some embodiments of the foregoing, Ring A is pyridinyl. In some embodiments of the foregoing, X is N, n is 1, and R² is H.

In some embodiments of Formula (III), X is N; Y is CR⁴; R⁴ is H; and n, R², R³, Ring A, and Ring B are as detailed herein for Formula (I). In some embodiments of Formula (III), X is N; Y is CR⁴; R⁴ is H; Ring B is C₃-C₁₀ cycloalkyl, 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl; and n, R², R³, and Ring A are as detailed herein for Formula (I). In some embodiments of Formula (III), X is N; Y is CR⁴; R⁴ is H; Ring B is 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl; and n, R², R³, and Ring A are as detailed herein for Formula (I). In some embodiments of Formula (III), X is N; Y is CR⁴; R⁴ is H; n is 1; R² and R³ are each H; Ring A is pyridyl; Ring B is 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH3, and phenyl.

In some embodiments of Formula (III), X and Y are each N; Ring B is C₃-C₁₀ cycloalkyl, 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH3, and phenyl; and n, R², R³, and Ring A are as detailed herein for Formula (I). In some embodiments of Formula (III), X and Y are each N; Ring B is C₃-C₁₀ cycloalkyl, 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl; n is 1; R² and R³ are each H; and Ring A is as detailed herein for Formula (I). In some embodiments of Formula (III), X and Y are each N; Ring B is 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl; n is 1; R² and R³ are each H; and Ring A is as detailed herein for Formula (I). In some embodiments of Formula (III), X and Y are each N; Ring B is C₃-C₁₀ cycloalkyl, 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl; n is 1; R² and R³ are each H; and Ring A is pyrazolyl or pyridyl, each of which is optionally substituted by halo, CN, C₃-C₆ cycloalkyl, or C₁-C₆ alkyl optionally substituted by halo or OH.

In some embodiments, provided is a compound of Formula (IV):

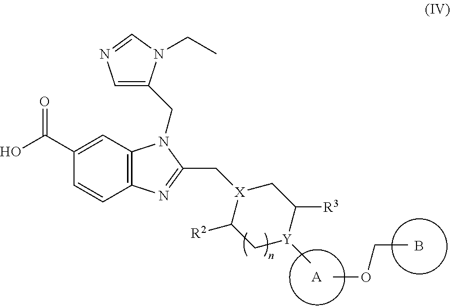

or a stereoisomer, tautomer, or a pharmaceutically acceptable salt of any of the foregoing, wherein X, Y, n, R², R³, Ring A, and Ring B are as detailed herein for Formula (I) with the provisos if applicable. In some embodiments of Formula (III) or (IV), both X and Y are N, n is 1, both R² and R³ are oxo, and the compound is of Formula (III-a) or (IV-a),

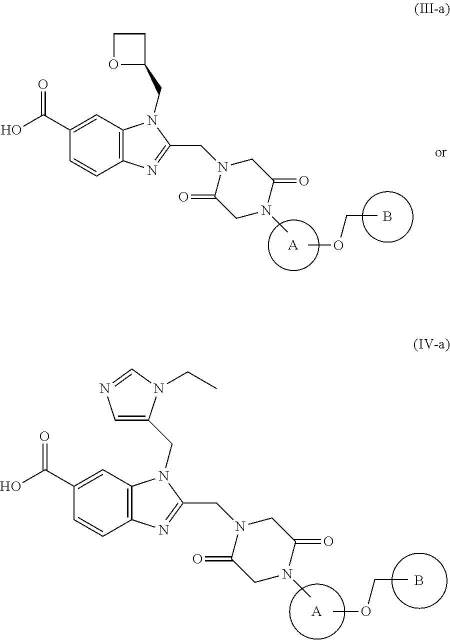

or a stereoisomer, tautomer, or a pharmaceutically acceptable salt of any of the foregoing, wherein Ring A, and Ring B are as detailed herein for Formula (I). In certain embodiments of Formula (III-a) or (IV-a), Ring A is pyridinyl.

In some embodiments of Formula (III) or (IV), X is N, Y is CR⁴, n is 1, R² is H, R³ and R⁴ taken together with the carbon atoms to which they are attached to form cyclopropyl, and the compound is of Formula (III-b) or (IV-b),

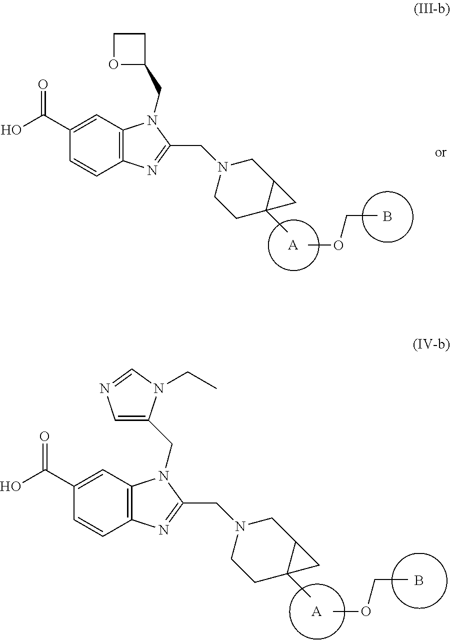

or a stereoisomer, tautomer, or a pharmaceutically acceptable salt of any of the foregoing, wherein Ring A, and Ring B are as detailed herein for Formula (I). In certain embodiments of Formula (III-b) or (IV-b), Ring A is pyridinyl. In certain embodiments of Formula (III-b) or (IV-b), Ring A is pyrazolyl. In certain embodiments of Formula (III-b) or (IV-b), Ring B is optionally substituted phenyl. In certain embodiments of Formula (III-b) or (IV-b), Ring A is pyridinyl and Ring B is as detailed herein for Formula (I). In certain embodiments of Formula (III-b) or (IV-b), Ring A is pyrazolyl and Ring B is as detailed herein for Formula (I). In certain embodiments of Formula (III-b) or (IV-b), Ring A is pyridinyl and Ring B is optionally substituted phenyl. In certain embodiments of Formula (III-b) or (IV-b), Ring A is pyrazolyl and Ring B is optionally substituted phenyl.

In some embodiments of Formula (III) or (IV), X is N, Y is CH, n is 1, both R² and R³ are hydrogen, and Ring A is

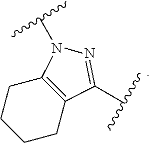 [SMILES: *c1nn(*)c2c1CCCC2]

In some embodiments of Formula (III) or (IV), X is N, Y is CH, n is 1, both R² and R³ are hydrogen, Ring A is pyridinyl, and Ring B is 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl.

In some embodiments, provided is a compound of Formula (IV′):

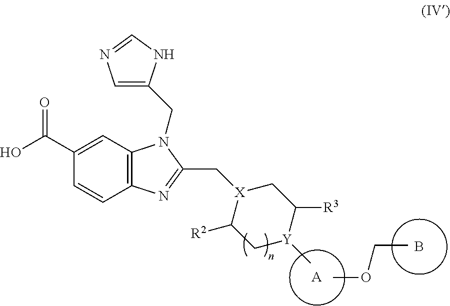

or a pharmaceutically acceptable salt thereof, wherein X, Y, n, R², R³, Ring A, and Ring B are as detailed herein for Formula (I) with the provisos if applicable.

In some embodiments, provided is a compound of Formula (IV″):

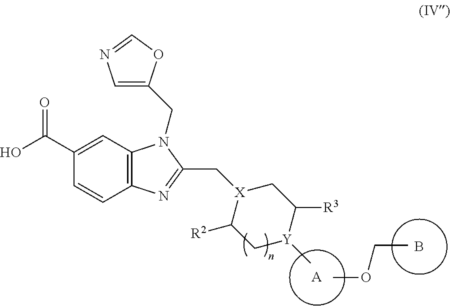

or a pharmaceutically acceptable salt thereof, wherein X, Y, n, R², R³, Ring A, and Ring B are as detailed herein for Formula (I) with the provisos if applicable.

In some embodiments of Formula (IV′) or (IV″), both X and Y are N, n is 1, both
R² and R³ are oxo, and the compound is of Formula (IV′-a) or (IV″-a),

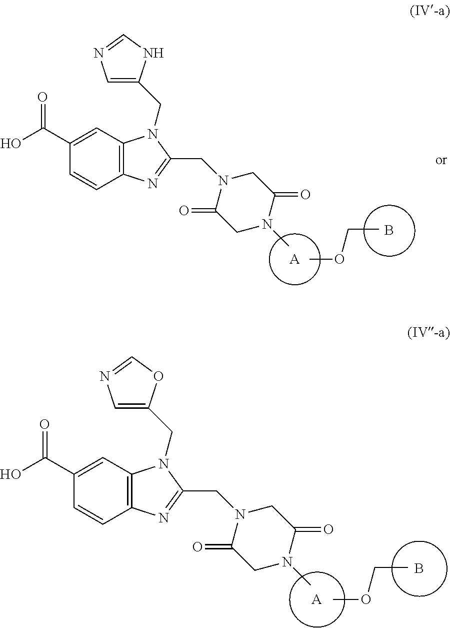

or a pharmaceutically acceptable salt thereof, wherein Ring A, and Ring B are as detailed herein for Formula (I). In certain embodiments of Formula (III-a) or (IV-a), Ring A is pyridinyl.

In some embodiments of Formula (IV′) or (IV″), X is N, Y is CR⁴, n is 1, R² is H, R³ and R⁴ taken together with the carbon atoms to which they are attached to form cyclopropyl, and the compound is of Formula (IV′-b) or (IV″-b),

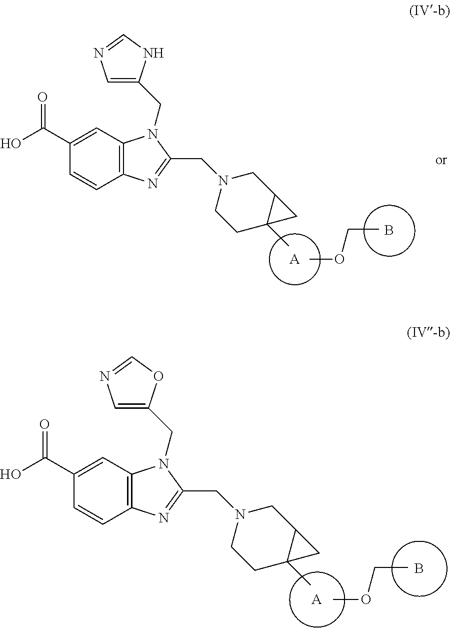

or a pharmaceutically acceptable salt thereof, wherein Ring A, and Ring B are as detailed herein for Formula (I). In certain embodiments of Formula (IV′-b) or (IV″-b), Ring A is pyridinyl. In certain embodiments of Formula (IV′-b) or (IV″-b), Ring A is pyrazolyl. In certain embodiments of Formula (IV′-b) or (IV″-b), Ring B is optionally substituted phenyl. In certain embodiments of Formula (IV′-b) or (IV″-b), Ring A is pyridinyl and Ring B is as detailed herein for Formula (I). In certain embodiments of Formula (IV′-b) or (IV″-b), Ring A is pyrazolyl and Ring B is as detailed herein for Formula (I). In certain embodiments of Formula (IV′-b) or (IV″-b), Ring A is pyridinyl and Ring B is optionally substituted phenyl. In certain embodiments of Formula (IV′-b) or (IV″-b), Ring A is pyrazolyl and Ring B is optionally substituted phenyl.

In some embodiments of Formula (IV′) or (IV″), X is N, Y is CH, n is 1, both R² and R³ are hydrogen, and Ring A is

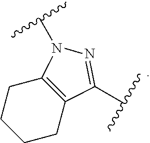 [SMILES: *c1nn(*)c2c1CCCC2]

In some embodiments of Formula (IV′) or (IV″), X is N, Y is CH, n is 1, both R² and R³ are hydrogen, Ring A is pyridinyl, and Ring B is 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl.

In some embodiments of a compound of Formula (I) (including compounds of Formulae (II)-(IV) if applicable), or a stereoisomer, tautomer, or a pharmaceutically acceptable salt of any of the foregoing, wherein R¹ is —C₁-C₆ alkylene-R⁵, wherein R⁵ is 3- to 6-membered heterocyclyl or 5- to 6-membered heteroaryl, each of which is independently optionally substituted by C₁-C₆ alkyl. In some embodiments, R¹ is —CH₂—R⁵. In another embodiment, R¹ is taken together with R and the intervening atoms to form a Ring C, wherein Ring C is a 5- to 7-membered heterocyclyl optionally substituted by C₁-C₆ alkyl. Exemplary Ring C include, but are not limited to,

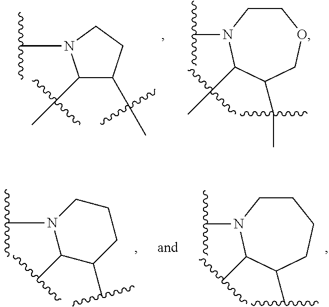

each of which is independently optionally substituted by C₁-C₆ alkyl.

In some embodiments of a compound of Formula (I) (including compounds of Formulae (II)-(IV) if applicable), or a stereoisomer, tautomer, or a pharmaceutically acceptable salt of any of the foregoing, wherein R⁵ is 3- to 6-membered heterocyclyl, optionally substituted by C₁-C₆ alkyl. For example, R⁵ is

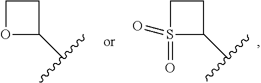 [SMILES: *C1CCO1 ; *C1CCS1(=O)=O]

each of which is independently optionally substituted by C₁-C₆ alkyl. In some embodiments, R⁵ is

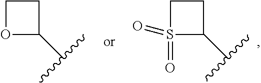 [SMILES: *C1CCO1 ; *C1CCS1(=O)=O]

each of which is independently optionally substituted by C₁-C₆ alkyl. In some embodiments, R⁵ is

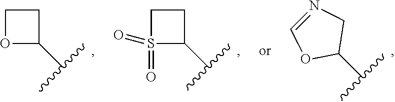 [SMILES: *C1CCO1 ; *C1CCS1(=O)=O ; *C1CN=CO1]

each of which is independently optionally substituted by C₁-C₆ alkyl. In some embodiments, R⁵ is 5- to 6-membered heteroaryl, optionally substituted by C₁-C₆ alkyl. In some embodiments, R⁵ is 5-membered heteroaryl, optionally substituted by C₁-C₆ alkyl. In some embodiments, R⁵ is

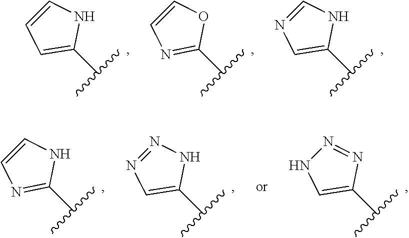 [SMILES: *c1ccc[nH]1 ; *c1ncco1 ; *c1cnc[nH]1 ; *c1ncc[nH]1 ; *c1cnn[nH]1 ; *c1c[nH]nn1]

each of which is optionally substituted by C₁-C₆ alkyl. In some embodiments, R⁵ is

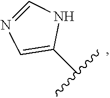 [SMILES: *c1cnc[nH]1]

which is optionally substituted by C₁-C₆ alkyl.

In some embodiments of a compound of Formula (I) (including compounds of Formulae (II)-(IV)), or a stereoisomer, tautomer, or a pharmaceutically acceptable salt of any of the foregoing, X is N. In other embodiments, X is CH.

In some embodiments of a compound of Formula (I) (including compounds of Formulae (II)-(IV)), or a stereoisomer, tautomer, or a pharmaceutically acceptable salt of any of the foregoing, n is 0. In other embodiments, n is 1.

In some embodiments of a compound of Formula (I) (including compounds of Formulae (II)-(IV)), or a stereoisomer, tautomer, or a pharmaceutically acceptable salt of any of the foregoing, Y is N. In other embodiments, Y is CR⁴, wherein R⁴ is hydrogen, OH or C₁-C₆ alkyl. In other embodiments, Y is CR⁴, and R³ and R⁴ are optionally taken together with the carbon atoms to which they are attached to form C₃-C₆ cycloalkyl. For example, the C₃-C₆ cycloalkyl can be cyclopropyl.

In some embodiments of a compound of Formula (I) (including compounds of Formulae (II)-(IV)), or a stereoisomer, tautomer, or a pharmaceutically acceptable salt of any of the foregoing, R² and R³ are independently hydrogen, oxo, or C₁-C₆ alkyl. In some embodiments, R² and R³ are hydrogen. In some embodiments, R² and R³ are oxo. In some embodiments, R² and R³ are methyl.

In some embodiments of a compound of Formula (I) (including compounds of Formulae (II)-(IV)), or a stereoisomer, tautomer, or a pharmaceutically acceptable salt of any of the foregoing, the moiety

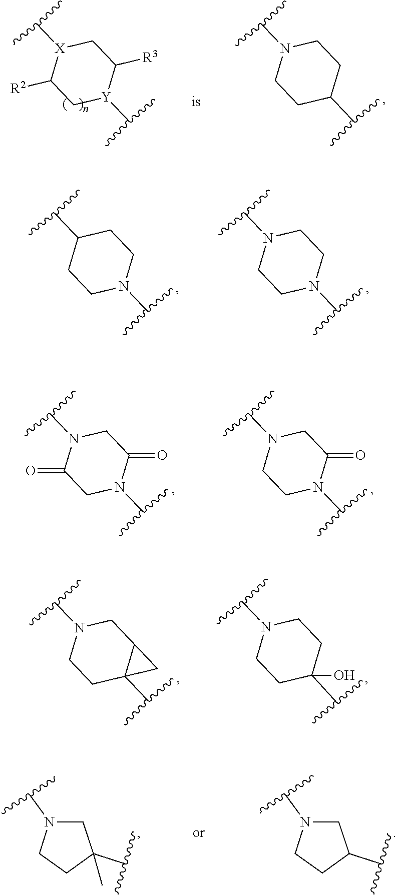

In some embodiments of a compound of Formula (I) (including compounds of Formulae (II)-(IV)), or a stereoisomer, tautomer, or a pharmaceutically acceptable salt of any of the foregoing, Ring A is 5- to 12-membered heterocyclyl, which is optionally substituted by halo, CN, C₃-C₆ cycloalkyl, or C₁-C₆ alkyl optionally substituted by halo or OH. For example, Ring A can be

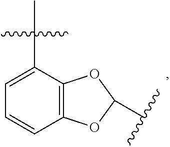 [SMILES: *c1cccc2c1OC(*)O2]

which is optionally substituted by halo, CN, C₃-C₆ cycloalkyl, or C₁-C₆ alkyl optionally substituted by halo or OH. In some embodiments, Ring A is

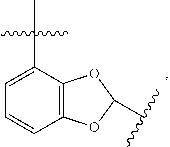 [SMILES: *c1cccc2c1OC(*)O2]

which is optionally substituted by halo, CN, C₃-C₆ cycloalkyl, or C₁-C₆ alkyl optionally substituted by halo or OH. In some embodiments, Ring A

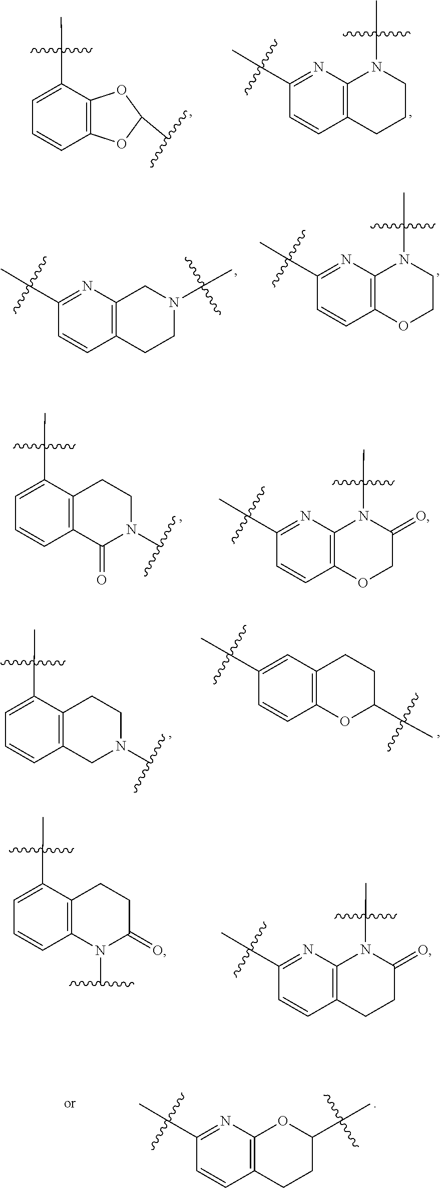

In some embodiments, Ring A is

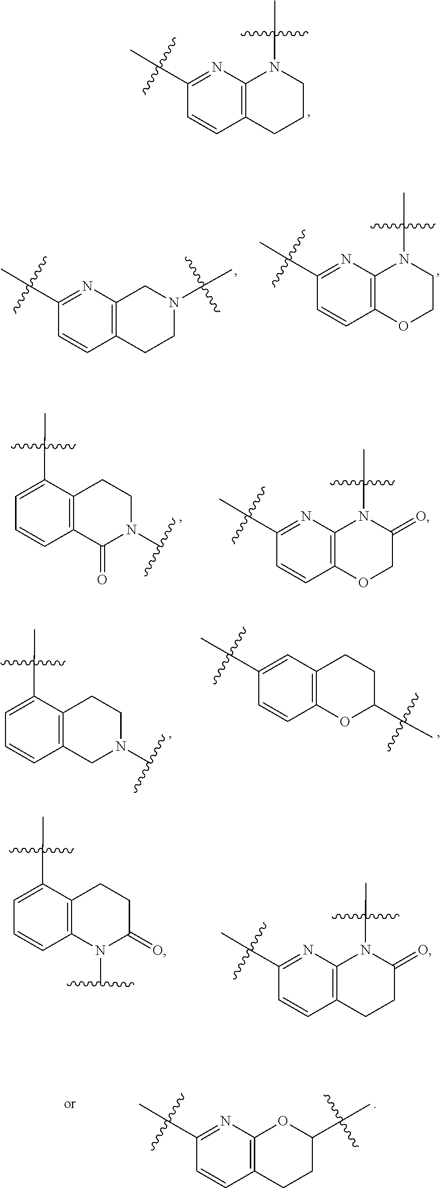

each of which is optionally substituted by halo, CN, C₃-C₆ cycloalkyl, or C₁-C₆ alkyl optionally substituted by halo or OH. In some embodiments,
Ring A is 5- to 12-membered heteroaryl, which is optionally substituted by halo, CN, C₃-C₆ cycloalkyl, or C₁-C₆ alkyl optionally substituted by halo or OH. Exemplary Ring A include, but are not limited to

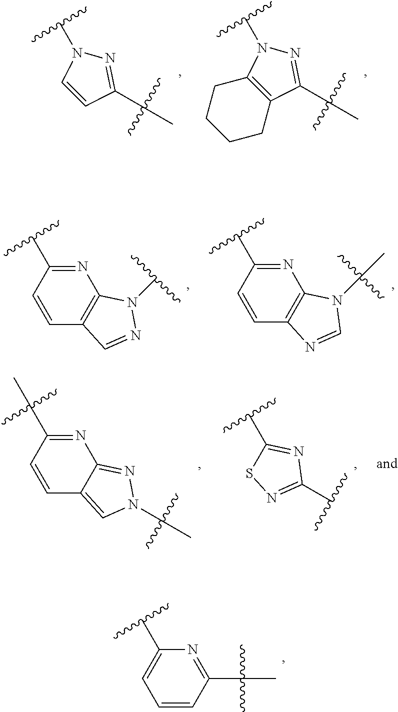

each of which is independently optionally substituted by halo, CN, C₃-C₆ cycloalkyl, or C₁-C₆ alkyl optionally substituted by halo or OH. In some embodiments, Ring A is

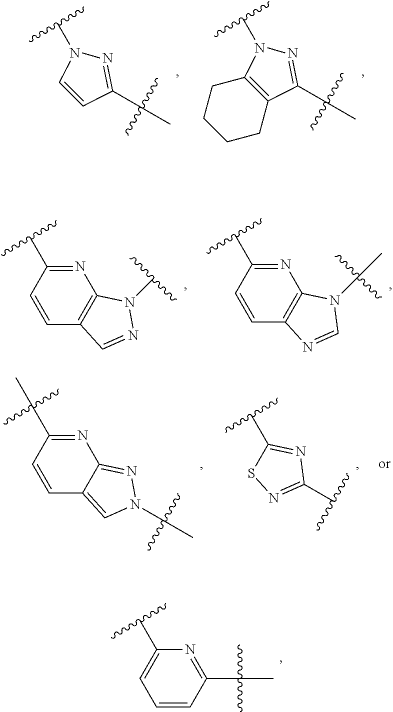

each of which is independently optionally substituted by halo, CN, C₃-C₆ cycloalkyl, or C₁-C₆ alkyl optionally substituted by halo or OH. In some embodiments, Ring A is

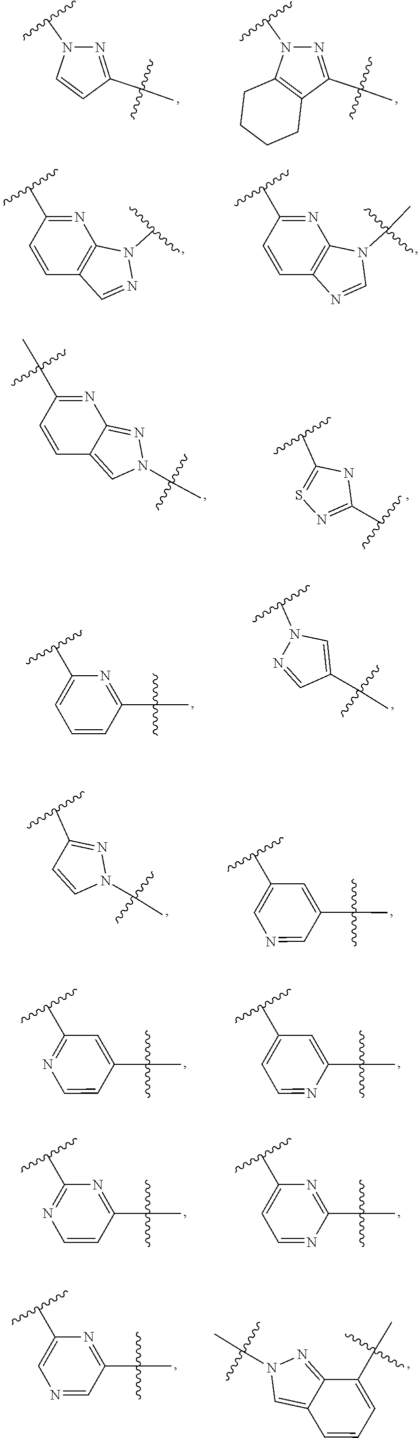

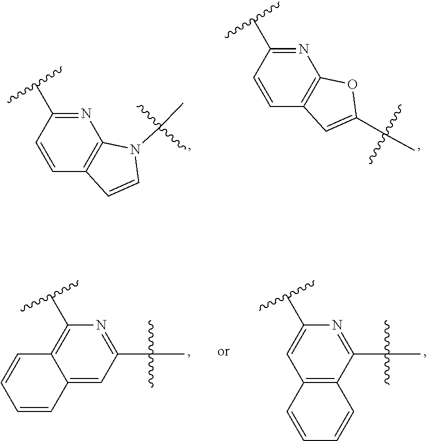 [SMILES: *c1ccc2ccn(*)c2n1 ; *c1ccc2cc(*)oc2n1 ; *c1cc2ccccc2c(*)n1 ; *c1cc2ccccc2c(*)n1]

each of which is independently optionally substituted by halo, CN, C₃-C₆ cycloalkyl, or C₁-C₆ alkyl optionally substituted by halo or OH. In some embodiments, Ring A is

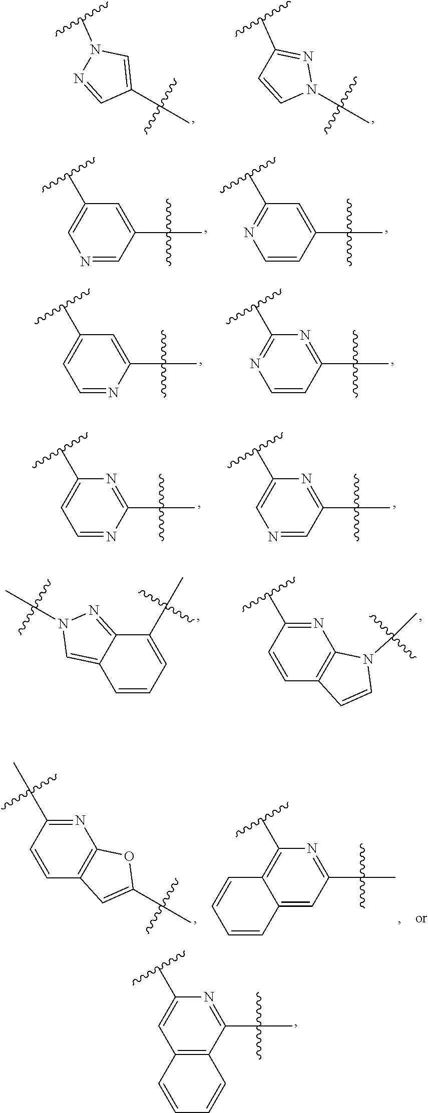

each of which is independently optionally substituted by halo, CN, C₃-C₆ cycloalkyl, or C₁-C₆ alkyl optionally substituted by halo or OH.

In some embodiments of a compound of Formula (I) (including compounds of Formulae (II)-(IV) if applicable), or a stereoisomer, tautomer, or a pharmaceutically acceptable salt of any of the foregoing, wherein L is bond. In some embodiments, L is —O—. In some embodiments, L is C₁-C₆ alkylene. In some embodiments, L is unsubstituted C₁-C₆ alkylene. In some embodiments, L is C₁-C₆ alkylene optionally substituted by R^{L1}, wherein each R^{L1} is independently halo, OH, or C₁-C₆ alkyl, or two R^{L1} are taken together with the carbon atom or atoms to which they are attached to form C₃-C₆ cycloalkyl or 3- to 6-membered heterocyclyl. In some embodiments, L is unsubstituted C₁-C₂ alkylene. In some embodiments, L is C₁-C₂ alkylene optionally substituted by R^{L1}, wherein each R^{L1} is independently halo, OH, or C₁-C₆ alkyl. In some embodiments, L is unsubstituted C₂ alkylene. In some embodiments, L is C₂ alkylene optionally substituted by R^{L1}, wherein each R^{L1} is independently halo, OH, or C₁-C₆ alkyl. In some such embodiments, L is

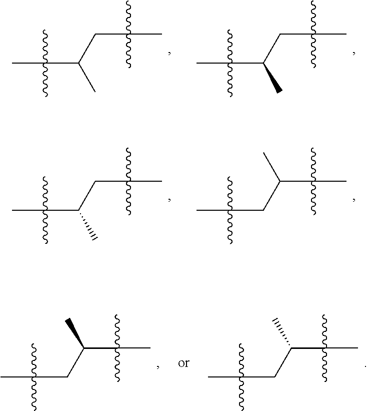

In some embodiments, L is *—O—C₁-C₆ alkylene-**, wherein * represents the point of attachment to ring A and ** represents the point of attachment to ring B. For example, L can be *—OCH₂—**. In some embodiments, when L is *—O—C₁-C₆ alkylene-**, the C₁-C₆ alkylene is substituted by R^{L}, wherein each R^{L} is independently C₁-C₆ alkyl or halo, or two R^{L} are taken together with the carbon atom or atoms to which they are attached to form C₃-C₆ cycloalkyl or 3- to 6-membered heterocyclyl. In some embodiments, when L is *—O—C₁-C₆ alkylene-**, the C₁-C₆ alkylene is substituted by R^{L}, wherein each R^{L} is independently C₁-C₆ alkyl or two R^{L} are taken together with the carbon atom or atoms to which they are attached to form C₃-C₆ cycloalkyl or 3- to 6-membered heterocyclyl. To give a specific example, when L is *—OC(RL)₂-**, two RL can be taken together with the carbon atom or atoms to which they are attached to form C₃-C₆ cycloalkyl or 3- to 6-membered heterocyclyl. In some embodiments, L is *—C₁-C₆ alkylene-O—**. In some embodiments, L is *—NR⁶—C₁-C₆ alkylene-**, wherein R⁶ is hydrogen or C₁-C₆ alkyl.

In some embodiments of a compound of Formula (I) (including compounds of Formulae (II)-(IV)), or a stereoisomer, tautomer, or a pharmaceutically acceptable salt of any of the foregoing, wherein Ring B is C₃-C₁₀ cycloalkyl, which is optionally substituted by one to three substituents independently selected from the group consisting of halo, CN, oxo, C₁-C₆ alkyl, —COCH₃, —CONH₂, —S(O)₂CH₃ and phenyl. Exemplary C₃-C₁₀ cycloalkyl include, but are not limited to,

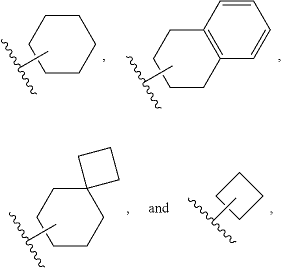

each of which is independently optionally substituted by one to three substituents independently selected from the group consisting of halo, CN, oxo, C₁-C₆ alkyl, —COCH₃, —CONH₂, —S(O)₂CH₃ and phenyl. In some embodiments, Ring B is

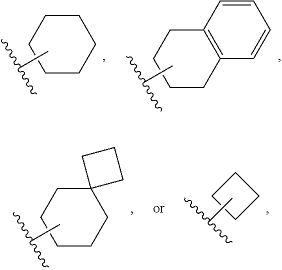

each of which is independently optionally substituted by one to three substituents independently selected from the group consisting of halo, CN, oxo, C₁-C₆ alkyl, —COCH₃, —CONH₂, —S(O)₂CH₃ and phenyl. In some embodiments, Ring B is

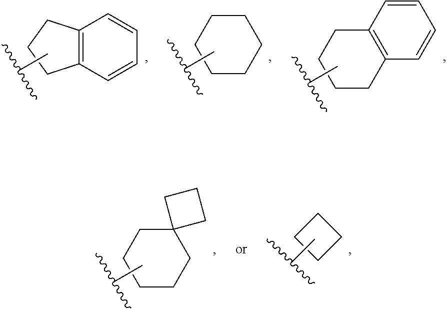

each of which is independently optionally substituted by one to three substituents independently selected from the group consisting of halo, CN, oxo, C₁-C₆ alkyl, —COCH₃, —CONH₂, —S(O)₂CH₃ and phenyl. In some embodiments, Ring B is C₆-C₁₄ aryl, which is optionally substituted by one to three substituents independently selected from the group consisting of halo, CN, oxo, C₁-C₆ alkyl, —COCH₃, —CONH₂, —S(O)₂CH₃ and phenyl. For example, the C₆-C₁₄ aryl can be

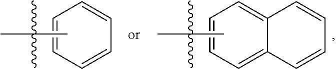

or each of which is independently optionally substituted by one to three substituents each independently selected from the group consisting of halo, CN, oxo, C₁-C₆ alkyl, —COCH₃, —CONH₂, —S(O)₂CH₃ and phenyl. In some embodiments, Ring B is 4- to 12-membered heterocyclyl, which is optionally substituted by one to three substituents independently selected from the group consisting of halo, CN, oxo, C₁-C₆ alkyl, —COCH₃, —CONH₂, —S(O)₂CH₃ and phenyl. Exemplary 4- to 12-membered heterocyclyl include, but are not limited to,

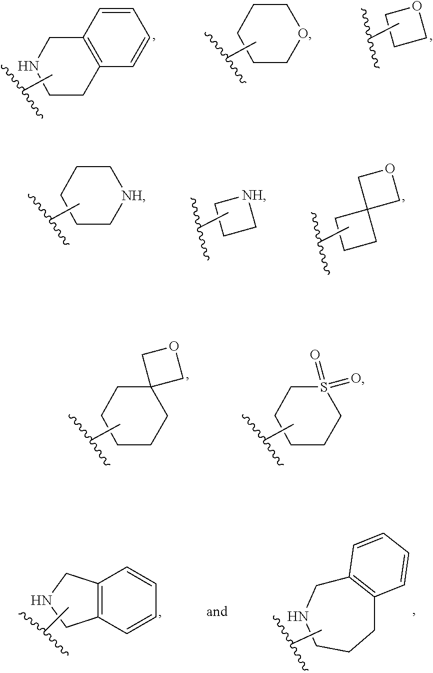

each of which is independently optionally substituted by one to three substituents independently selected from the group consisting of halo, CN, oxo, C₁-C₆ alkyl, —COCH₃, —CONH₂, —S(O)₂CH₃ and phenyl. In some embodiments, Ring B is

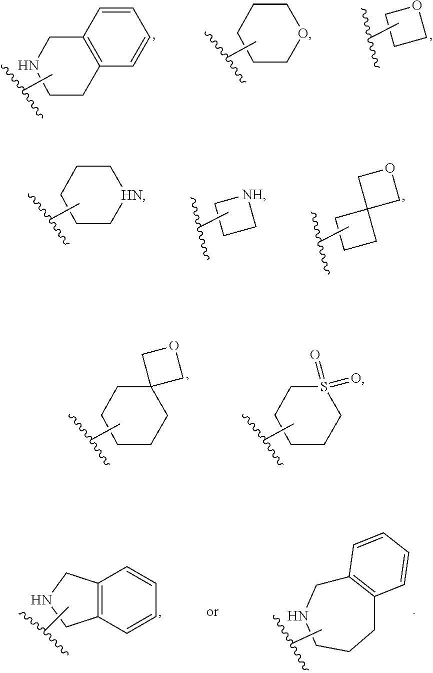

In some embodiments, Ring B is

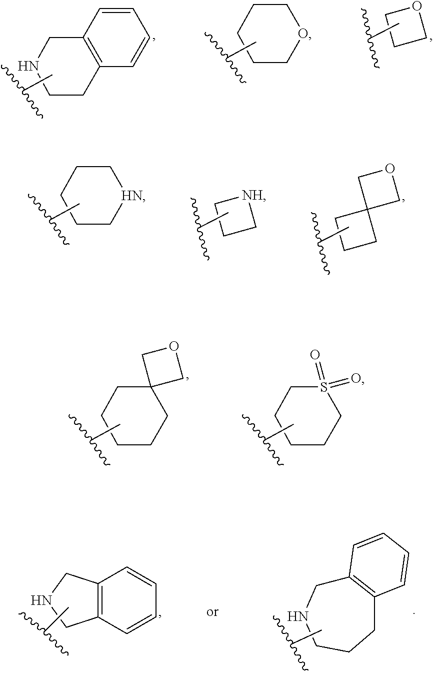

In some embodiments, Ring B is 5- to 12-membered heteroaryl, which is optionally substituted by one to three substituents independently selected from the group consisting of halo, CN, oxo, C₁-C₆ alkyl, —COCH₃, —CONH₂, —S(O)₂CH₃ and phenyl. Exemplary 5- to 12-membered heteroaryl include, but are not limited to

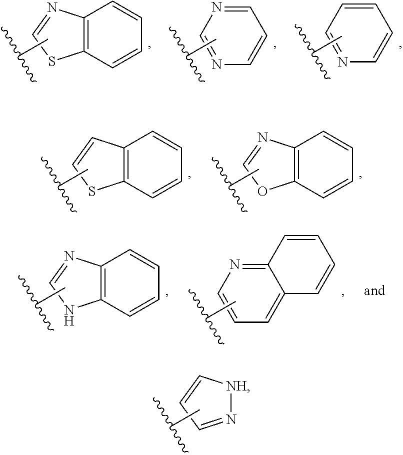

each of which is independently optionally substituted by one to three substituents independently selected from the group consisting of halo, CN, oxo, C₁-C₆ alkyl, —COCH₃, —CONH₂, —S(O)₂CH₃ and phenyl. In some embodiments, Ring B is

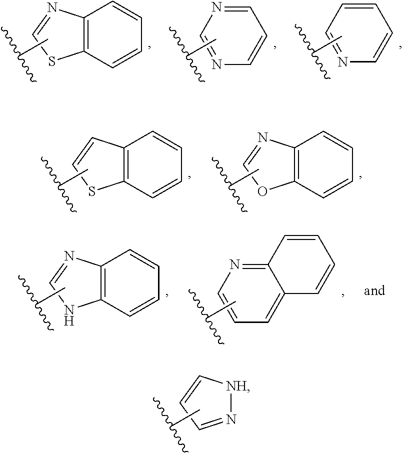

each of which is independently optionally substituted by one to three substituents independently selected from the group consisting of halo, CN, oxo, C₁-C₆ alkyl, —COCH₃, —CONH₂, —S(O)₂CH₃ and phenyl. In some embodiments, Ring B is

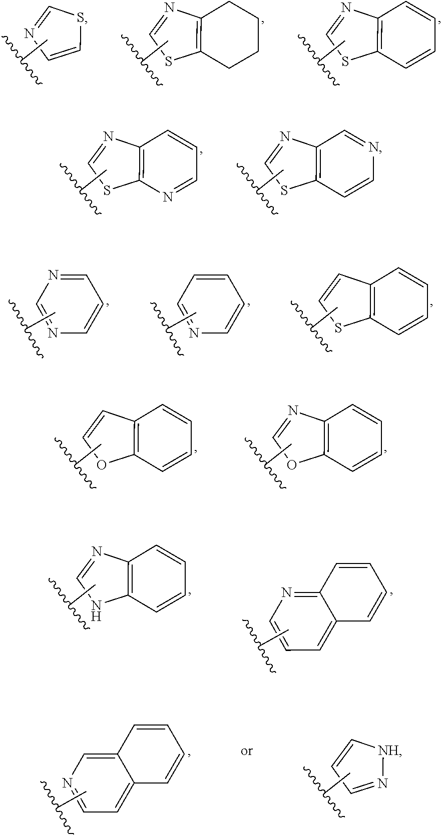

each of which is independently optionally substituted by one to three substituents independently selected from the group consisting of halo, CN, oxo, C₁-C₆ alkyl, —COCH₃, —CONH₂, —S(O)₂CH₃ and phenyl. In some embodiments, Ring B is

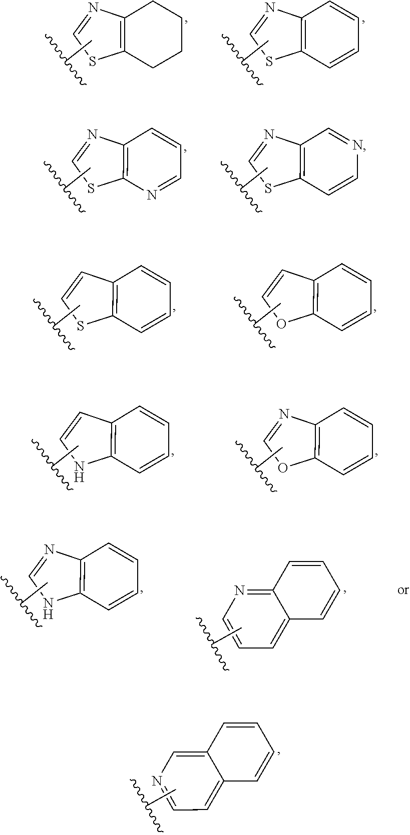

each of which is independently optionally substituted by one to three substituents independently selected from the group consisting of halo, CN, oxo, C₁-C₆ alkyl, —COCH₃, —CONH₂, —S(O)₂CH₃ and phenyl.

In some embodiments, of Formula (I), provided is a compound of Formula (V):

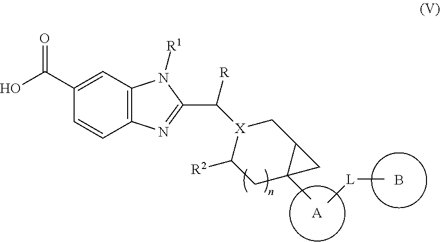

or a pharmaceutically acceptable salt thereof, wherein X, n, R, R¹, R², Ring A, L, and Ring B are as described for Formula (I). In some such embodiments of Formula (V), Ring B is C₃-C₁₀ cycloalkyl, C₆-C₁₄ aryl, 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl.

In some embodiments of Formula (V), the compound is of Formula (Va) or (Vb):

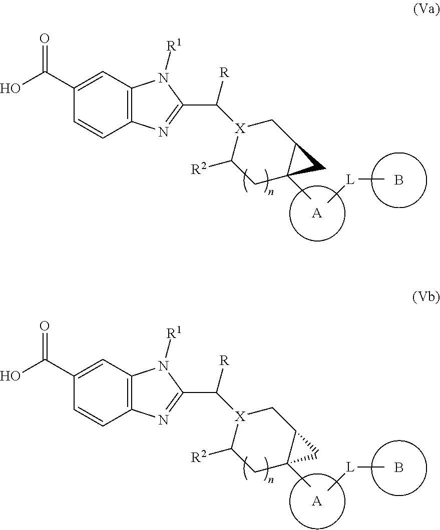

or a pharmaceutically acceptable salt thereof, wherein X, n, R, R¹, R², Ring A, L, and Ring B are as described for Formula (I). In some such embodiments of Formula (Va) or (Vb), Ring B is C₃-C₁₀ cycloalkyl, C₆-C₁₄ aryl, 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl.

In some embodiments of Formula (I), (V), (Va), or (Vb), X is N, n is 1, and R, R¹, R², Ring A, L, and Ring B are as described for Formula (I). In some embodiments of Formula (I), (V), (Va), or (Vb), X is N, n is 1, R¹ is —CH₂—R⁵, and R, R⁵, R², Ring A, L, and Ring B are as described for Formula (I). In some such embodiments, R⁵ is 3- to 6-membered heterocyclyl, optionally substituted by C₁-C₆ alkyl. In other such embodiments, R⁵ is 5- to 6-membered heteroaryl optionally substituted by C₁-C₆ alkyl, preferably wherein R⁵ is 5-membered heteroaryl, optionally substituted by C₁-C₆ alkyl.

In some embodiments of Formula (I), (V), (Va), or (Vb), X is N, n is 1, R¹ is —CH₂—R⁵, R⁵ is 3- to 6-membered heterocyclyl optionally substituted by C₁-C₆ alkyl, Ring A is 5- to 12-membered heterocyclyl, which is optionally substituted by halo, CN, C₃-C₆ cycloalkyl, or C₁-C₆ alkyl optionally substituted by halo or OH; and R, R², L, and Ring B are as described for Formula (I). In some embodiments of Formula (I), (V), (Va), or (Vb), X is N, n is 1, R¹ is —CH₂—R⁵, R⁵ is 3- to 6-membered heterocyclyl optionally substituted by C₁-C₆ alkyl, Ring A is 9- to 10-membered heterocyclyl, which is optionally substituted by halo, CN, C₃-C₆ cycloalkyl, or C₁-C₆ alkyl optionally substituted by halo or OH; and R, R², L, and Ring B are as described for Formula (I). In some embodiments of any of the foregoing, R⁵ is 4- to 5-membered heterocyclyl optionally substituted by C₁-C₆ alkyl.

In some embodiments of Formula (I), (V), (Va), or (Vb), X is N, n is 1, R¹ is —CH₂—R⁵, R⁵ is 3- to 6-membered heterocyclyl optionally substituted by C₁-C₆ alkyl, Ring A is 5- to 12-membered heteroaryl, which is optionally substituted by halo, CN, C₃-C₆ cycloalkyl, or C₁-C₆ alkyl optionally substituted by halo or OH; and R, R², L, and Ring B are as described for Formula (I). In some embodiments of Formula (I), (V), (Va), or (Vb), X is N, n is 1, R¹ is —CH₂—R⁵, R⁵ is 3- to 6-membered heterocyclyl optionally substituted by C₁-C₆ alkyl, Ring A is 5- to 6-membered heteroaryl, which is optionally substituted by halo, CN, C₃-C₆ cycloalkyl, or C₁-C₆ alkyl optionally substituted by halo or OH; and R, R², L, and Ring B are as described for Formula (I). In some such embodiments, Ring A is 5- to 6-membered heteroaryl, which is optionally substituted by halo, CN, C₃-C₆ cycloalkyl, or C₁-C₆ alkyl optionally substituted by halo or OH. In some such embodiments, Ring A is 6-membered heteroaryl, which is optionally substituted by halo, CN, C₃-C₆ cycloalkyl, or C₁-C₆ alkyl optionally substituted by halo or OH. In some such embodiments, Ring A is unsubstituted 6-membered heteroaryl such as

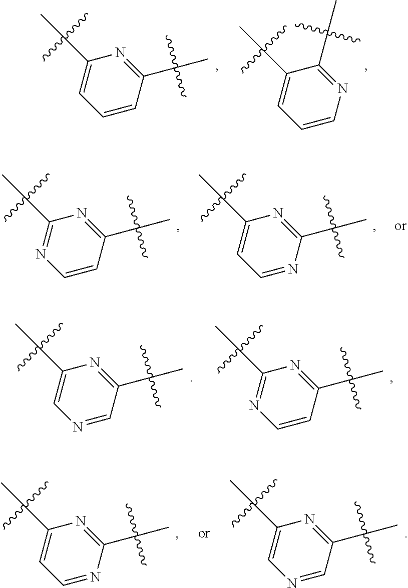

In some such embodiments, Ring A is, N, or N. In some embodiments of any of the foregoing, R⁵ is 4- to 5-membered heterocyclyl optionally substituted by C₁-C₆ alkyl. In some embodiments of Formula (I), (V), (Va), or (Vb), X is N, n is 1, R¹ is —CH₂—R⁵, R⁵ is 5- to 6-membered heteroaryl optionally substituted by C₁-C₆ alkyl, Ring A is 5- to 12-membered heterocyclyl, which is optionally substituted by halo, CN, C₃-C₆ cycloalkyl, or C₁-C₆ alkyl optionally substituted by halo or OH; and R, R², L, and Ring B is as described for Formula (I). In some embodiments of Formula (I), (V), (Va), or (Vb), X is N, n is 1, R¹ is —CH₂—R⁵, R⁵ is 5- to 6-membered heteroaryl optionally substituted by C₁-C₆ alkyl, Ring A is 9- to 10-membered heterocyclyl, which is optionally substituted by halo, CN, C₃-C₆ cycloalkyl, or C₁-C₆ alkyl optionally substituted by halo or OH; and R, R², L, and Ring B is as described for Formula (I).

In some embodiments of Formula (I), (V), (Va), or (Vb), X is N, n is 1, R¹ is —CH₂—R⁵, R⁵ is 5-membered heteroaryl optionally substituted by C₁-C₆ alkyl, Ring A is 9- to 10-membered heterocyclyl, which is optionally substituted by halo, CN, C₃-C₆ cycloalkyl, or C₁-C₆ alkyl optionally substituted by halo or OH; and R, R², L, and Ring B is as described for Formula (I).

In some embodiments of Formula (I), (V), (Va), or (Vb), X is N, n is 1, R¹ is —CH₂—R⁵, R⁵ is 5- to 6-membered heteroaryl optionally substituted by C₁-C₆ alkyl, Ring A is 5- to 12-membered heteroaryl, which is optionally substituted by halo, CN, C₃-C₆ cycloalkyl, or C₁-C₆ alkyl optionally substituted by halo or OH; and R, R², L, and Ring B are is as described for Formula (I). In some embodiments of Formula (I), (V), (Va), or (Vb), X is N, n is 1, R¹ is —CH₂—R⁵, R⁵ is 5- to 6-membered heteroaryl optionally substituted by C₁-C₆ alkyl, Ring A is 5- to 6-membered heteroaryl, which is optionally substituted by halo, CN, C₃-C₆ cycloalkyl, or C₁-C₆ alkyl optionally substituted by halo or OH; and R, R², L, and Ring B are is as described for Formula (I).

In some embodiments of Formula (I), (V), (Va), or (Vb), X is N, n is 1, R¹ is —CH₂—R⁵, R⁵ is 5-membered heteroaryl optionally substituted by C₁-C₆ alkyl, Ring A is 5- to 6-membered heteroaryl, which is optionally substituted by halo, CN, C₃-C₆ cycloalkyl, or C₁-C₆ alkyl optionally substituted by halo or OH; and R, R², L, and Ring B are is as described for Formula (I). In some such embodiments, Ring A is 6-membered heteroaryl, which is optionally substituted by halo, CN, C₃-C₆ cycloalkyl, or C₁-C₆ alkyl optionally substituted by halo or OH. In some such embodiments, Ring A is

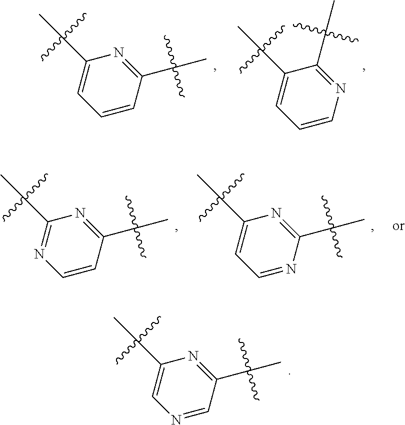 [SMILES: *c1cccc(*)n1 ; *c1cccnc1* ; *c1ccnc(*)n1 ; *c1ccnc(*)n1 ; *c1cncc(*)n1]

In some embodiments of Formula (I), (V), (Va), or (Vb), X is N, n is 1, R¹ is —CH₂—R⁵, R⁵ is 3- to 6-membered heterocyclyl optionally substituted by C₁-C₆ alkyl, Ring B is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I).

In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl.

In some embodiments of Formula (I), (V), (Va), or (Vb), X is N, n is 1, R¹ is —CH₂—R⁵, R⁵ is 4- to 5-membered heterocyclyl optionally substituted by C₁-C₆ alkyl, Ring B is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I).

In some such embodiments, Ring B is cyclobutyl, cyclohexyl, or tetrahydronaphthalenyl, each of which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl. In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl.

In some embodiments of Formula (I), (V), (Va), or (Vb), X is N, n is 1, R¹ is —CH₂—R⁵, R⁵ is 3- to 6-membered heterocyclyl optionally substituted by C₁-C₆ alkyl, Ring B is C₆-C₁₄ aryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl.

In some embodiments of Formula (I), (V), (Va), or (Vb), X is N, n is 1, R¹ is —CH₂—R⁵, R⁵ is 4- to 5-membered heterocyclyl optionally substituted by C₁-C₆ alkyl, Ring B is C₆-C₁₄ aryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some such embodiments, Ring B is phenyl or naphthalenyl, each of which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl. In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl.

In some such embodiments, Ring B is

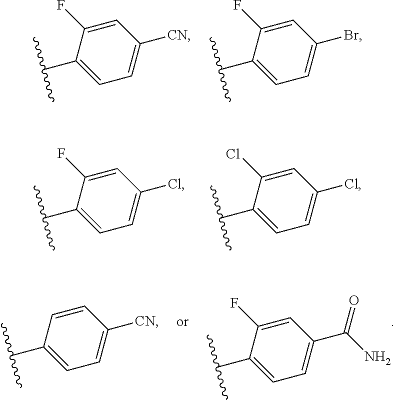 [SMILES: *c1ccc(C#N)cc1F ; *c1ccc(Br)cc1F ; *c1ccc(Cl)cc1F ; *c1ccc(Cl)cc1Cl ; *c1ccc(C#N)cc1 ; *c1ccc(C(N)=O)cc1F]

In some embodiments of Formula (I), (V), (Va), or (Vb), X is N, n is 1, R¹ is —CH₂—R⁵, R⁵ is 3- to 6-membered heterocyclyl optionally substituted by C₁-C₆ alkyl, Ring B is 4- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl.

In some embodiments of Formula (I), (V), (Va), or (Vb), X is N, n is 1, R¹ is —CH₂—R⁵, R⁵ is 4- to 5-membered heterocyclyl optionally substituted by C₁-C₆ alkyl, Ring B is 9- to 10-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some such embodiments, Ring B is

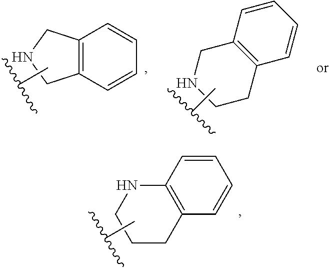

each of which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl. In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl.

In some embodiments of Formula (I), (V), (Va), or (Vb), X is N, n is 1, R¹ is —CH₂—R⁵, R⁵ is 3- to 6-membered heterocyclyl optionally substituted by C₁-C₆ alkyl, Ring B is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl.

In some embodiments of Formula (I), (V), (Va), or (Vb), X is N, n is 1, R¹ is —CH₂—R⁵, R⁵ is 4- to 5-membered heterocyclyl optionally substituted by C₁-C₆ alkyl, Ring B is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl.

In some embodiments of Formula (I), (V), (Va), or (Vb), X is N, n is 1, R¹ is —CH₂—R⁵, R⁵ is 4- to 5-membered heterocyclyl optionally substituted by C₁-C₆ alkyl, Ring B is 9- to 10-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl.

In some embodiments of Formula (I), (V), (Va), or (Vb), X is N, n is 1, R¹ is —CH₂—R⁵, R⁵ is 5- to 6-membered heteroaryl optionally substituted by C₁-C₆ alkyl, Ring B is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl.

In some embodiments of Formula (I), (V), (Va), or (Vb), X is N, n is 1, R¹ is —CH₂—R⁵, R⁵ is 5-membered heteroaryl optionally substituted by C₁-C₆ alkyl, Ring B is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; Ring B is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², and L are as described for Formula (I). In some such embodiments, Ring A is

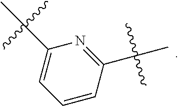 [SMILES: *c1cccc(*)n1]

In other such embodiments, Ring A is

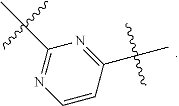 [SMILES: *c1ccnc(*)n1]

In still other such embodiments, Ring A is

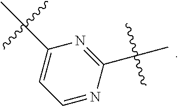 [SMILES: *c1ccnc(*)n1]

In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is *—O—C₁-C₆ alkylene-**; Ring B is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). In some such embodiments, L is *—O—CH₂—**. For example, in some embodiments

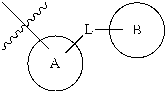

is

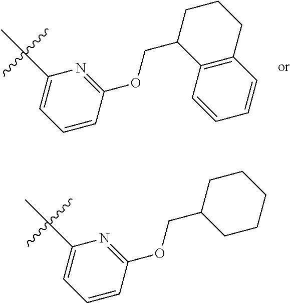 [SMILES: *c1cccc(OCC2CCCc3ccccc32)n1 ; *c1cccc(OCC2CCCCC2)n1]

or the like, optionally substituted as described for Ring A, L, and Ring B herein. In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is a bond; Ring B is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). For example, in some embodiments,

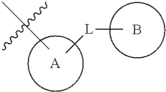

is

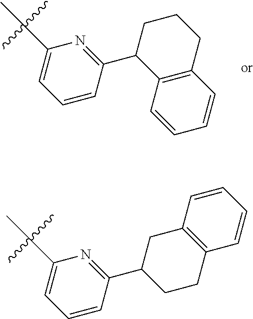 [SMILES: *c1cccc(C2CCCc3ccccc32)n1 ; *c1cccc(C2CCc3ccccc3C2)n1]

or the like, optionally substituted as described for Ring A and Ring B herein. In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is —O—; Ring B is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). For example, in some embodiments

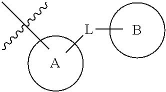

is

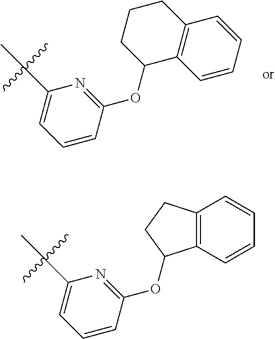 [SMILES: *c1cccc(OC2CCCc3ccccc32)n1 ; *c1cccc(OC2CCc3ccccc32)n1]

or the like, optionally substituted as described for Ring A and Ring B herein. In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring B is C₆-C₁₄ aryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of Formula (I), (V), (Va), or (Vb), X is N, n is 1, R¹ is —CH₂—R⁵, R⁵ is 5-membered heteroaryl optionally substituted by C₁-C₆ alkyl, Ring B is C₆-C₁₄ aryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of Formula (I), (V), (Va), or (Vb), X is N, n is 1, R¹ is —CH₂—R⁵, R⁵ is 5-membered heteroaryl optionally substituted by C₁-C₆ alkyl, Ring B is C₆ aryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; Ring B is C₆ aryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², and L are as described for Formula (I). In some such embodiments, Ring A is

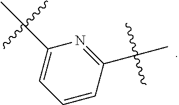 [SMILES: *c1cccc(*)n1]

In other such embodiments, Ring A is

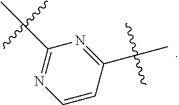 [SMILES: *c1ccnc(*)n1]

In still other such embodiments, Ring A is

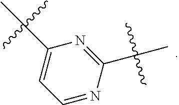 [SMILES: *c1ccnc(*)n1]

In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is *—O—C₁-C₆ alkylene-**; Ring B is C₆ aryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). In some such embodiments, L is *—O—CH₂—**. For example, in some embodiments,

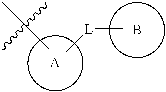

is

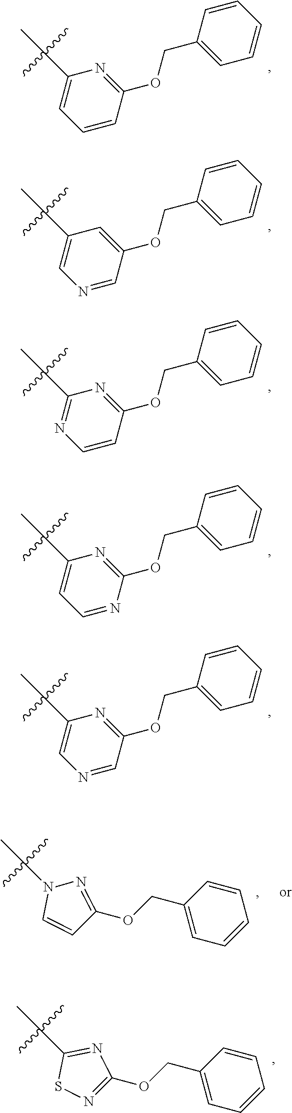

or the like, optionally substituted as described for Ring A, L, and Ring B herein. For example, in some embodiments,

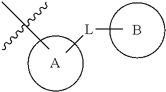

is

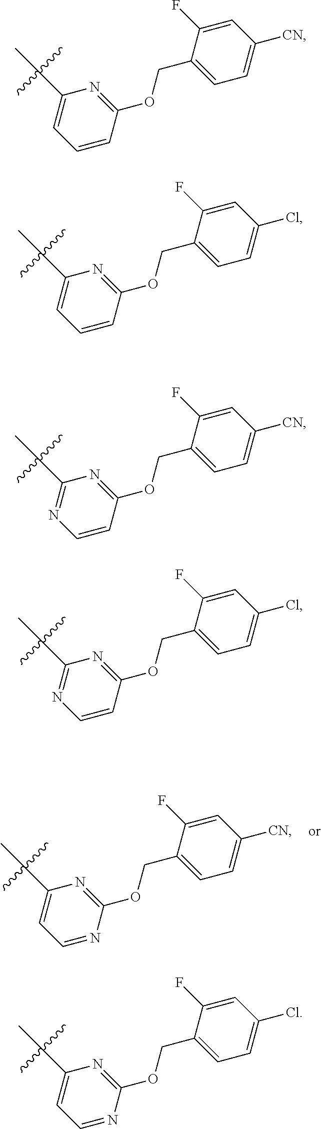 [SMILES: *c1cccc(OCc2ccc(C#N)cc2F)n1 ; *c1cccc(OCc2ccc(Cl)cc2F)n1 ; *c1nccc(OCc2ccc(C#N)cc2F)n1 ; *c1nccc(OCc2ccc(Cl)cc2F)n1 ; *c1ccnc(OCc2ccc(C#N)cc2F)n1 ; *c1ccnc(OCc2ccc(Cl)cc2F)n1]

In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is a bond; Ring B is C₆ aryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). In some embodiments of the foregoing, Ring B is C₃-C₁₀ cycloalkyl, which is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is —O—; Ring B is C₆ aryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). In some embodiments of the foregoing, Ring B is C₃-C₁₀ cycloalkyl, which is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring B is 4- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some such embodiments, Ring A is

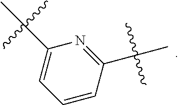 [SMILES: *c1cccc(*)n1]

In other such embodiments, Ring A is

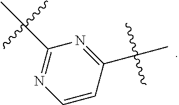 [SMILES: *c1ccnc(*)n1]

In still other such embodiments, Ring A is

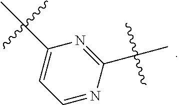 [SMILES: *c1ccnc(*)n1]

In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring B is 4- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of Formula (I), (V), (Va), or (Vb), Ring B is 9- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; Ring B is 4- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², and L are as described for Formula (I). In some such embodiments, Ring A is

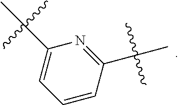 [SMILES: *c1cccc(*)n1]

In other such embodiments, Ring A is

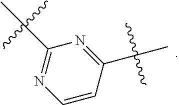 [SMILES: *c1ccnc(*)n1]

In still other such embodiments, Ring A is

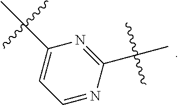 [SMILES: *c1ccnc(*)n1]

In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is *—O—C₁-C₆ alkylene-**; Ring B is 4- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). In some such embodiments, L is *—O—CH₂—**. For example, in some embodiments of Formula (I), (V), (Va), or (Vb),

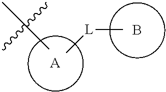

is

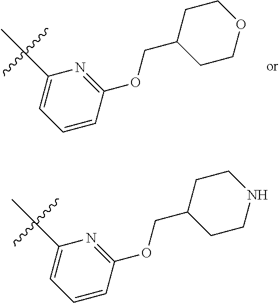 [SMILES: *c1cccc(OCC2CCOCC2)n1 ; *c1cccc(OCC2CCNCC2)n1]

or the like, optionally substituted as described for Ring A and Ring B herein. In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is a bond; Ring B is 9- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). For example, in some embodiments,

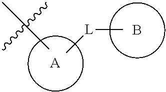

is

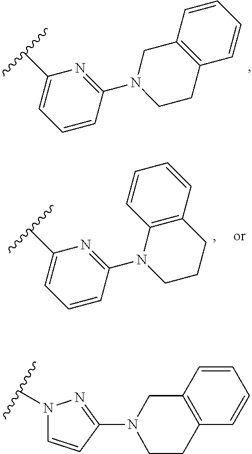 [SMILES: *c1cccc(N2CCc3ccccc3C2)n1 ; *c1cccc(N2CCCc3ccccc32)n1 ; *n1ccc(N2CCc3ccccc3C2)n1]

or the like, optionally substituted as described for Ring A and Ring B herein. In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is —O—; Ring B is 9- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring B is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of Formula (I), (V), (Va), or (Vb), Ring B is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², Ring A, and L are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; Ring B is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², and L are as described for Formula (I). In some such embodiments, Ring A is

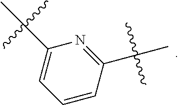 [SMILES: *c1cccc(*)n1]

In other such embodiments, Ring A is

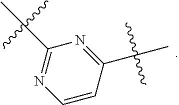 [SMILES: *c1ccnc(*)n1]

In still other such embodiments, Ring A is

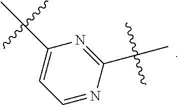 [SMILES: *c1ccnc(*)n1]

In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is *—O—C₁-C₆ alkylene-**; Ring B is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). In some such embodiments, L is *—O—CH₂—**. For example, in some embodiments of Formula (I), (V), (Va), or (Vb),

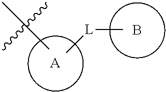

is

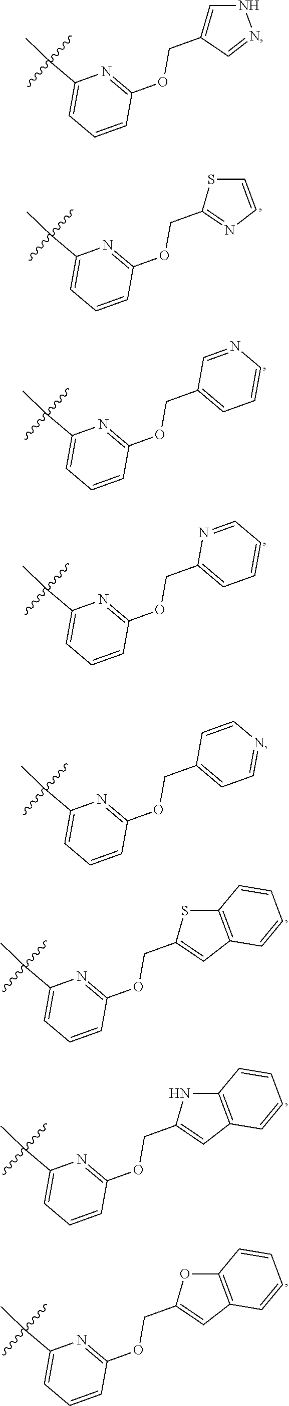

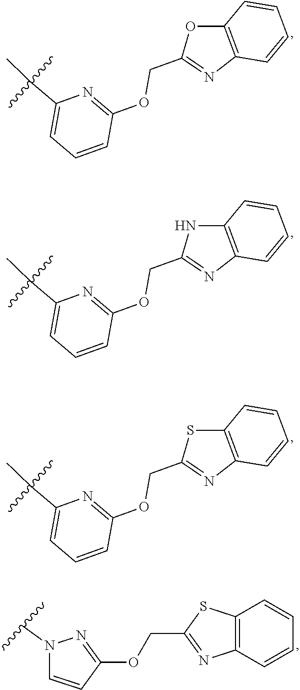 [SMILES: *c1cccc(OCc2nc3ccccc3o2)n1 ; *c1cccc(OCc2nc3ccccc3[nH]2)n1 ; *c1cccc(OCc2nc3ccccc3s2)n1 ; *n1ccc(OCc2nc3ccccc3s2)n1]

or the like, optionally substituted as described for Ring A, L, and Ring B herein. In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is a bond; Ring B is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (I), (V), (Va), or (Vb), Ring A is 5- to 6-membered heteroaryl, which is optionally substituted by halo, CN, C₃-C₆ cycloalkyl, or C₁-C₆ alkyl optionally substituted by halo or OH; L is —O—; Ring B is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R and R² are as described for Formula (I). In some embodiments of the foregoing, Ring B is optionally substituted by one to three substituents independently selected from the group consisting of halo, CN, oxo, C₁-C₆ alkyl, —COCH₃, —CONH₂, —S(O)₂CH₃ and phenyl. In some embodiments of any of the foregoing, X is N, n is 1, R¹ is —CH₂—R⁵, R⁵ is 5-membered heteroaryl optionally substituted by C₁-C₆ alkyl.

In some embodiments of Formula (Vb), the compound is of Formula (Vb-1):

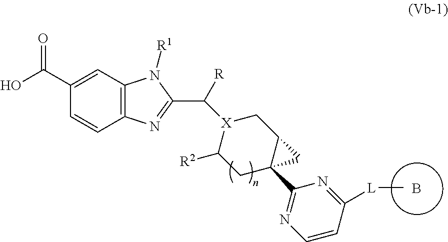

or a pharmaceutically acceptable salt thereof, wherein X, n, R, R¹, R², L, and Ring B are as described for Formula (I). In some such embodiments of Formula (Vb-1), Ring B is C₃-C₁₀ cycloalkyl, C₆-C₁₄ aryl, 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl.

In some embodiments of Formula (Vb-1), X is N, n is 1, and R, R¹, R², L, and Ring B are as described for Formula (I). In some embodiments of Formula (Vb-1), X is N, n is 1, R¹ is —CH₂—R⁵, and R, R⁵, R², L, and Ring B are as described for Formula (I). In some such embodiments, R⁵ is 3- to 6-membered heterocyclyl, optionally substituted by C₁-C₆ alkyl. In other such embodiments, R⁵ is 5- to 6-membered heteroaryl optionally substituted by C₁-C₆ alkyl, preferably wherein R⁵ is 5-membered heteroaryl, optionally substituted by C₁-C₆ alkyl.

In some embodiments of Formula (Vb-1), X is N, n is 1, R¹ is —CH₂—R⁵, R⁵ is 5-membered heteroaryl optionally substituted by C₁-C₆ alkyl, Ring B is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², and L are as described for Formula (I). In some such embodiments, L is *—O—C₁-C₆ alkylene-**, preferably wherein L is *—O—CH₂—**. In other such embodiments, L is a bond. In other such embodiments, L is —O—.

In some embodiments of Formula (Vb-1), X is N, n is 1, R¹ is —CH₂—R⁵, R⁵ is 5-membered heteroaryl optionally substituted by C₁-C₆ alkyl, Ring B is C₃-C₁₀ cycloalkyl, which is optionally substituted by one to three substituents independently selected from the group consisting of halo, CN, oxo, C₁-C₆ alkyl, —COCH₃, —CONH₂, —S(O)₂CH₃ and phenyl; and R, R², and L are as described for Formula (I). In some such embodiments, L is *—O—C₁-C₆ alkylene-**, preferably wherein L is *—O—CH₂—**. In other such embodiments, L is a bond. In other such embodiments, L is —O—.

In some embodiments of Formula (Vb-1), X is N, n is 1, R¹ is —CH₂—R⁵, R⁵ is 5-membered heteroaryl optionally substituted by C₁-C₆ alkyl, Ring B is C₆-C₁₄ aryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², and L are as described for Formula (I). In some such embodiments, L is *—O—C₁-C₆ alkylene-**, preferably wherein L is *—O—CH₂—**. In other such embodiments,
L is a bond. In other such embodiments, L is —O—.

In some embodiments of Formula (Vb-1), X is N, n is 1, R¹ is —CH₂—R⁵, R⁵ is 5-membered heteroaryl optionally substituted by C₁-C₆ alkyl, Ring B is C₆-C₁₄ aryl, which is optionally substituted by one to three substituents independently selected from the group consisting of halo, CN, oxo, C₁-C₆ alkyl, —COCH₃, —CONH₂, —S(O)₂CH₃ and phenyl; and R, R², and L are as described for Formula (I). In some such embodiments, L is *—O—C₁-C₆ alkylene-**, preferably wherein L is *—O—CH₂—**. In other such embodiments, L is a bond. In other such embodiments, L is —O—.

In some embodiments of Formula (Vb-1), X is N, n is 1, R¹ is —CH₂—R⁵, R⁵ is 5-membered heteroaryl optionally substituted by C₁-C₆ alkyl, Ring B is 4- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², and L are as described for Formula (I). In some such embodiments, L is *—O—C₁-C₆ alkylene-**, preferably wherein L is *—O—CH₂—**. In other such embodiments, L is a bond. In other such embodiments, L is —O—.

In some embodiments of Formula (Vb-1), X is N, n is 1, R¹ is —CH₂—R⁵, R⁵ is 5-membered heteroaryl optionally substituted by C₁-C₆ alkyl, Ring B is 4- to 12-membered heterocyclyl, which is optionally substituted by one to three substituents independently selected from the group consisting of halo, CN, oxo, C₁-C₆ alkyl, —COCH₃, —CONH₂, —S(O)₂CH₃ and phenyl; and R, R², and L are as described for Formula (I). In some such embodiments, L is *—O—C₁-C₆ alkylene-**, preferably wherein L is *—O—CH₂—**. In other such embodiments, L is a bond. In other such embodiments, L is —O—.

In some embodiments of Formula (Vb-1), X is N, n is 1, R¹ is —CH₂—R⁵, R⁵ is 5-membered heteroaryl optionally substituted by C₁-C₆ alkyl, Ring B is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; and R, R², and L are as described for Formula (I). In some such embodiments, L is *—O—C₁-C₆ alkylene-**, preferably wherein L is *—O—CH₂—**. In other such embodiments, L is a bond. In other such embodiments, L is —O—.

In some embodiments of Formula (Vb-1), X is N, n is 1, R¹ is —CH₂—R⁵, R⁵ is 5-membered heteroaryl optionally substituted by C₁-C₆ alkyl, Ring B is 5- to 12-membered heteroaryl, which is optionally substituted by one to three substituents independently selected from the group consisting of halo, CN, oxo, C₁-C₆ alkyl, —COCH₃, —CONH₂, —S(O)₂CH₃ and phenyl; and R, R², and L are as described for Formula (I). In some such embodiments, L is *—O—C₁-C₆ alkylene-**, preferably wherein L is *—O—CH₂—**. In other such embodiments, L is a bond. In other such embodiments, L is —O—.

In some embodiments, provided is a compound of Formula (I′):

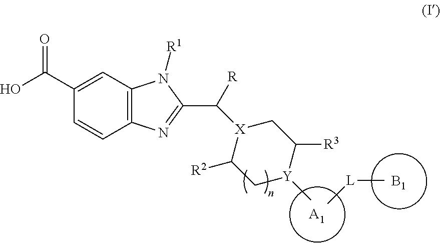

or a pharmaceutically acceptable salt thereof, wherein
X, Y, n, R, R¹, R², R³, and L are as described for Formula (I);
Ring A₁ is 5- to 12-membered heteroaryl optionally substituted by halo, CN, C₃-C₆ cycloalkyl, or C₁-C₆ alkyl optionally substituted by halo or OH; and
Ring B₁ is C₃-C₁₀ cycloalkyl, 4- to 12-membered heterocyclyl, or 5- to 12-membered heteroaryl, each of which is independently optionally substituted by one to three substituents independently selected from the group consisting of halo, CN, oxo, C₁-C₆ alkyl, C₁-C₆ haloalkyl, —COCH₃, —CONH₂, —S(O)₂CH₃, and phenyl. In some such embodiments of Formula (I′), Ring B₁ is C₃-C₁₀ cycloalkyl, 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl.

In some embodiments of Formula (I′), X is N. In some embodiments of Formula (I′), X is N, R¹ is —CH₂—R⁵; Y, n, R, R⁵, R², R³, and L are as described for Formula (I); and Ring A₁ and Ring B₁ are as described for Formula (I′).

In some embodiments of Formula (I′), X is N, R¹ is —CH₂—R⁵; R⁵ is 3- to 6-membered heterocyclyl optionally substituted by C₁-C₆ alkyl; Ring B₁ is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; Y, n, R, R², and R³ are as described for Formula (I); and Ring A₁ is as described for Formula (I′). In some embodiments of Formula (I′), X is N; R¹ is —CH₂—R⁵; R⁵ is 3- to 6-membered heterocyclyl optionally substituted by C₁-C₆ alkyl; Ring B₁ is C₃-C₁₀ cycloalkyl, which is optionally substituted by one to three substituents independently selected from the group consisting of halo, CN, oxo, C₁-C₆ alkyl, —COCH₃, —CONH₂, —S(O)₂CH₃ and phenyl; Y, n, R, R², and R³ are as described for Formula (I); and Ring A₁ is as described for Formula (I′).

In some embodiments of Formula (I′), X is N; R¹ is —CH₂—R⁵; R⁵ is 3- to 6-membered heterocyclyl optionally substituted by C₁-C₆ alkyl; Ring B₁ is 4- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; Y, n, R, R², and R³ are as described for Formula (I); and Ring A₁ is as described for Formula (I′). In some embodiments of Formula (I′), X is N; R¹ is —CH₂—R⁵; R⁵ is 3- to 6-membered heterocyclyl optionally substituted by C₁-C₆ alkyl; Ring B₁ is 4- to 12-membered heterocyclyl, which is optionally substituted by one to three substituents independently selected from the group consisting of halo, CN, oxo, C₁-C₆ alkyl, —COCH₃, —CONH₂, —S(O)₂CH₃ and phenyl; Y, n, R, R², and R³ are as described for Formula (I); and Ring A₁ is as described for Formula (I′).

In some embodiments of Formula (I′), X is N; R¹ is —CH₂—R⁵; R⁵ is 3- to 6-membered heterocyclyl optionally substituted by C₁-C₆ alkyl; Ring B₁ is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; Y, n, R, R², and R³ are as described for Formula (I); and Ring A₁ is as described for Formula (I′). In some embodiments of Formula (I′), X is N; R¹ is —CH₂—R⁵; R⁵ is 3- to 6-membered heterocyclyl optionally substituted by C₁-C₆ alkyl; Ring B₁ is 5- to 12-membered heteroaryl, which is optionally substituted by one to three substituents independently selected from the group consisting of halo, CN, oxo, C₁-C₆ alkyl, —COCH₃, —CONH₂, —S(O)₂CH₃ and phenyl; Y, n, R, R², and R³ are as described for Formula (I); and Ring A₁ is as described for Formula (I′).

In some embodiments of Formula (I′), X is N; R¹ is —CH₂—R⁵; R⁵ is 5- to 6-membered heteroaryl optionally substituted by C₁-C₆ alkyl; Ring B₁ is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; Y, n, R, R², and R³ are as described for Formula (I); and Ring A₁ is as described for Formula (I′). In some embodiments of Formula (I′), X is N; R¹ is —CH₂—R⁵; R⁵ is 5- to 6-membered heteroaryl optionally substituted by C₁-C₆ alkyl; Ring B₁ is C₃-C₁₀ cycloalkyl, which is optionally substituted by one to three substituents independently selected from the group consisting of halo, CN, oxo, C₁-C₆ alkyl, —COCH₃, —CONH₂, —S(O)₂CH₃ and phenyl; Y, n, R, R², and R³ are as described for Formula (I); and Ring A₁ is as described for Formula (I′).

In some embodiments of Formula (I′), X is N; R¹ is —CH₂—R⁵; R⁵ is 5- to 6-membered heteroaryl optionally substituted by C₁-C₆ alkyl; Ring B₁ is 4- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; Y, n, R, R², and R³ are as described for Formula (I); and Ring A₁ is as described for Formula (I′). In some embodiments of Formula (I′), X is N; R¹ is —CH₂—R⁵; R⁵ is 5- to 6-membered heteroaryl optionally substituted by C₁-C₆ alkyl; Ring B₁ is 4- to 12-membered heterocyclyl, which is optionally substituted by one to three substituents independently selected from the group consisting of halo, CN, oxo, C₁-C₆ alkyl, —COCH₃, —CONH₂, —S(O)₂CH₃ and phenyl; Y, n, R, R², and R³ are as described for Formula (I); and Ring A₁ is as described for Formula (I′).

In some embodiments of Formula (I′), X is N; R¹ is —CH₂—R⁵; 5- to 6-membered heteroaryl optionally substituted by C₁-C₆ alkyl; Ring B₁ is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl; Y, n, R, R², and R³ are as described for Formula (I); and Ring A₁ is as described for Formula (I′). In some embodiments of Formula (I′), X is N, R¹ is —CH₂—R⁵, R⁵ is 5- to 6-membered heteroaryl optionally substituted by C₁-C₆ alkyl; Ring B₁ is 5- to 12-membered heteroaryl, which is optionally substituted by one to three substituents independently selected from the group consisting of halo, CN, oxo, C₁-C₆ alkyl, —COCH₃, —CONH₂, —S(O)₂CH₃ and phenyl; Y, n, R, R², and R³ are as described for Formula (I); and Ring A₁ is as described for Formula (I′).

In some embodiments, provided is a compound of Formula (I″),

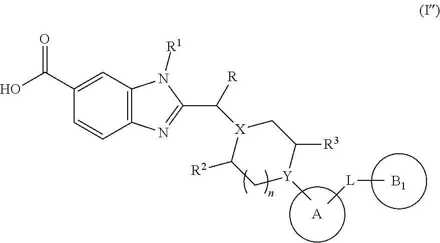

or a pharmaceutically acceptable salt thereof, wherein
X, Y, n, R, R², R³, Ring A, and L are as described for Formula (I);
R¹ is —C₁-C₆ alkylene-R^{5a}, wherein R^{5a} is 5- to 6-membered heteroaryl optionally substituted by C₁-C₆ alkyl; and
Ring B₁ is C₃-C₁₀ cycloalkyl, 4- to 12-membered heterocyclyl, or 5- to 12-membered heteroaryl, each of which is independently optionally substituted by one to three substituents independently selected from the group consisting of halo, CN, oxo, C₁-C₆ alkyl, C₁-C₆ haloalkyl, —COCH₃, —CONH₂, —S(O)₂CH₃, and phenyl. In some such embodiments of Formula (I′), Ring B₁ is C₃-C₁₀ cycloalkyl, 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl.

In some embodiments of Formula (I″), X is N. In some embodiments of Formula (I″), X is N, R¹ is —CH₂—R^{5a}; Y, n, R, R², R³, Ring A, and L are as described for Formula (I); and R^{5a} and Ring B₁ are as described for Formula (I″).

In some embodiments, provided is a compound of Formula (VI),

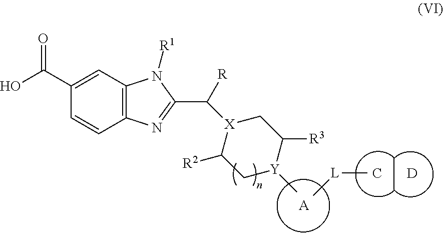

or a pharmaceutically acceptable salt thereof, wherein
X, Y, n, R, R¹, R², R³, Ring A, and L are as described for Formula (I);

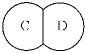

is a fused bicyclic ring system comprising fused rings Ring C and Ring D, wherein Ring C is C₅-C₆ cycloalkyl, 5- to 7-membered heterocyclyl, or 5- to 6-membered heteroaryl; and
   Ring D is C₆ cycloalkyl, C₆ aryl or 6-membered heteroaryl;
   wherein Ring C and Ring D are optionally substituted by one to three substituents independently selected from the group consisting of halo, CN, oxo, C₁-C₆ alkyl, C₁-C₆ haloalkyl, —COCH₃, —CONH₂, —S(O)₂CH₃, and phenyl. In some embodiments, Ring C and Ring D are optionally substituted by one to three substituents independently selected from the group consisting of halo, CN, oxo, C₁-C₆ alkyl, —COCH₃, —CONH₂, —S(O)₂CH₃, and phenyl.

In some embodiments of Formula (VI), Ring D is C₆ aryl, and Ring C is C₅-C₆ cycloalkyl, 5- to 7-membered heterocyclyl, or 5- to 6-membered heteroaryl, wherein Ring C and Ring D are optionally substituted by one to three substituents independently selected from the group consisting of halo, CN, oxo, C₁-C₆ alkyl, C₁-C₆ haloalkyl, —COCH₃, —CONH₂, —S(O)₂CH₃, and phenyl. In some embodiments, Ring C and Ring D are optionally substituted by one to three substituents independently selected from the group consisting of halo, CN, oxo, C₁-C₆ alkyl, —COCH₃, —CONH₂, —S(O)₂CH₃, and phenyl. In some embodiments of any of the foregoing, Ring A is 5- to 6-membered heteroaryl, which is optionally substituted by halo, CN, C₃-C₆ cycloalkyl, or C₁-C₆ alkyl optionally substituted by halo or OH.

In some embodiments of Formula (VI), Ring D is C₆ aryl, and Ring C is C₅-C₆ cycloalkyl. In some such embodiments, Ring C and Ring D form

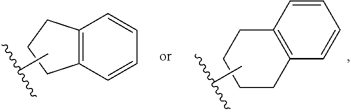

optionally substituted by one to three substituents independently selected from the group consisting of halo, CN, oxo, C₁-C₆ alkyl, C₁-C₆ haloalkyl, —COCH₃, —CONH₂, —S(O)₂CH₃, and phenyl. In some embodiments, Ring C and Ring D are optionally substituted by one to three substituents independently selected from the group consisting of halo, CN, oxo, C₁-C₆ alkyl, —COCH₃, —CONH₂, —S(O)₂CH₃, and phenyl. In some embodiments of any of the foregoing, X is N, n is 1, R¹ is —CH₂—R⁵, and R⁵ is 5- to 6-membered heteroaryl optionally substituted by C₁-C₆ alkyl. In some embodiments of any of the foregoing, X is N, n is 1, R¹ is —CH₂—R⁵, and R⁵ is 5-membered heteroaryl optionally substituted by C₁-C₆ alkyl.

In some embodiments of Formula (VI), Ring D is C₆ aryl and Ring C is 5- to 7-membered heterocyclyl. In some such embodiments, Ring C and Ring D form

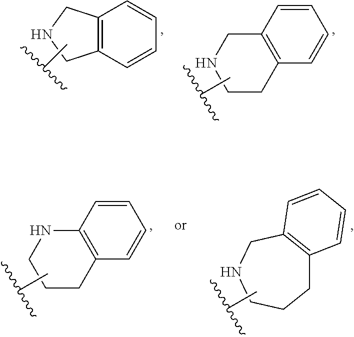

optionally substituted by one to three substituents independently selected from the group consisting of halo, CN, oxo, C₁-C₆ alkyl, C₁-C₆ haloalkyl, —COCH₃, —CONH₂, —S(O)₂CH₃, and phenyl. In some embodiments, Ring C and Ring D are optionally substituted by one to three substituents independently selected from the group consisting of halo, CN, oxo, C₁-C₆ alkyl, —COCH₃, —CONH₂, —S(O)₂CH₃, and phenyl. In some embodiments of any of the foregoing, X is N, n is 1, R¹ is —CH₂—R⁵, and R⁵ is 5- to 6-membered heteroaryl optionally substituted by C₁-C₆ alkyl. In some embodiments of any of the foregoing, X is N, n is 1, R¹ is —CH₂—R⁵, and R⁵ is 5-membered heteroaryl optionally substituted by C₁-C₆ alkyl.

In some embodiments of Formula (VI), Ring D is C₆ aryl and Ring C is 5- to 6-membered heteroaryl. In some such embodiments, Ring C and Ring D form

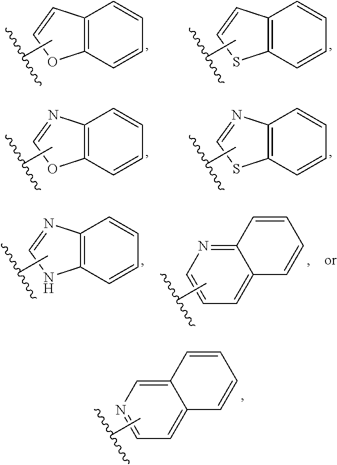

optionally substituted by one to three substituents independently selected from the group consisting of halo, CN, oxo, C₁-C₆ alkyl, C₁-C₆ haloalkyl, —COCH₃, —CONH₂, —S(O)₂CH₃, and phenyl. In some embodiments, Ring C and Ring D are optionally substituted by one to three substituents independently selected from the group consisting of halo, CN, oxo, C₁-C₆ alkyl, —COCH₃, —CONH₂, —S(O)₂CH₃, and phenyl. In some embodiments of any of the foregoing, X is N, n is 1, R¹ is —CH₂—R⁵, and R⁵ is 5- to 6-membered heteroaryl optionally substituted by C₁-C₆ alkyl. In some embodiments of any of the foregoing, X is N, n is 1, R¹ is —CH₂—R⁵, and R⁵ is 5-membered heteroaryl optionally substituted by C₁-C₆ alkyl.

In some embodiments of Formula (VI), Ring D is 6-membered heteroaryl and Ring C is C₅-C₆ cycloalkyl, 5- to 7-membered heterocyclyl, or 5- to 6-membered heteroaryl, wherein Ring C and Ring D are optionally substituted by one to three substituents independently selected from the group consisting of halo, CN, oxo, C₁-C₆ alkyl, C₁-C₆ haloalkyl, —COCH₃, —CONH₂, —S(O)₂CH₃, and phenyl. In some embodiments, Ring C and Ring D are optionally substituted by one to three substituents independently selected from the group consisting of halo, CN, oxo, C₁-C₆ alkyl, —COCH₃, —CONH₂, —S(O)₂CH₃, and phenyl. In some embodiments of any of the foregoing, X is N, n is 1, R¹ is —CH₂—R⁵, and R⁵ is 5- to 6-membered heteroaryl optionally substituted by C₁-C₆ alkyl. In some embodiments of any of the foregoing, X is N, n is 1, R¹ is —CH₂—R⁵, and R⁵ is 5-membered heteroaryl optionally substituted by C₁-C₆ alkyl.

In some embodiments of Formula (VI), Ring D is 6-membered heteroaryl and Ring C is C₅-C₆ cycloalkyl, wherein Ring C and Ring D are, optionally substituted by one to three substituents independently selected from the group consisting of halo, CN, oxo, C₁-C₆ alkyl, —COCH₃, —CONH₂, —S(O)₂CH₃, and phenyl. In some embodiments of any of the foregoing, X is N, n is 1, R¹ is —CH₂—R⁵, and R⁵ is 5- to 6-membered heteroaryl optionally substituted by C₁-C₆ alkyl. In some embodiments of any of the foregoing, X is N, n is 1, R¹ is —CH₂—R⁵, and R⁵ is 5-membered heteroaryl optionally substituted by C₁-C₆ alkyl.

In some embodiments of Formula (VI), Ring D is 6-membered heteroaryl and Ring C is 5- to 7-membered heterocyclyl, wherein Ring C and Ring D are, optionally substituted by one to three substituents independently selected from the group consisting of halo, CN, oxo, C₁-C₆ alkyl, —COCH₃, —CONH₂, —S(O)₂CH₃, and phenyl. In some embodiments of any of the foregoing, X is N, n is 1, R¹ is —CH₂—R⁵, and R⁵ is 5- to 6-membered heteroaryl optionally substituted by C₁-C₆ alkyl. In some embodiments of any of the foregoing, X is N, n is 1, R¹ is —CH₂—R⁵, and R⁵ is 5-membered heteroaryl optionally substituted by C₁-C₆ alkyl.

In some embodiments of Formula (VI), Ring D is 6-membered heteroaryl and Ring C is 5- to 6-membered heteroaryl. In some embodiments, Ring C and Ring D are

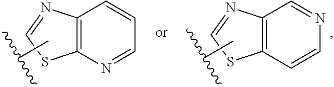

optionally substituted by one to three substituents independently selected from the group consisting of halo, CN, oxo, C₁-C₆ alkyl, —COCH₃, —CONH₂, —S(O)₂CH₃, and phenyl. In some embodiments of any of the foregoing, X is N, n is 1, R¹ is —CH₂—R⁵, and R⁵ is 5- to 6-membered heteroaryl optionally substituted by C₁-C₆ alkyl. In some embodiments of any of the foregoing, X is N, n is 1, R¹ is —CH₂—R⁵, and R⁵ is 5-membered heteroaryl optionally substituted by C₁-C₆ alkyl.

Representative compounds are listed in Table 1 below. In some embodiments, provided is a compound, or a stereoisomer, tautomer, or a pharmaceutically acceptable salt of any of the foregoing, which is selected from Compound Nos. 1-142 in Table 1. In some embodiments, provided is a compound, or a stereoisomer, tautomer, or a pharmaceutically acceptable salt of any of the foregoing, which is selected from Compound Nos. 143-187 in Table 1. In some embodiments, provided is a compound, or a stereoisomer, tautomer, or a pharmaceutically acceptable salt of any of the foregoing, which is selected from Compound Nos. 1-187 in Table 1. Compounds were prepared as described in the Examples.

TABLE 1

| Cmpd No. | Structure | Name |
|---|---|---|
| 1 | 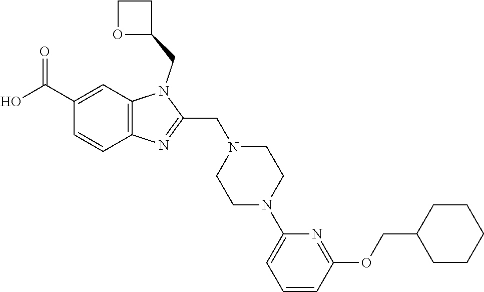 [SMILES: O=C(O)c1ccc2nc(CN3CCN(c4cccc(OCC5CCCCC5)n4)CC3)n(C[C@@H]3CCO3)c2c1] | (S)-2-((4-(6-(cyclohexylmethoxy)pyridin- 2-yl)piperazin-1-yl)methyl)-1-(oxetan-2- ylmethyl)-1H-benzo[d]imidazole-6- carboxylic acid |
| 2 | 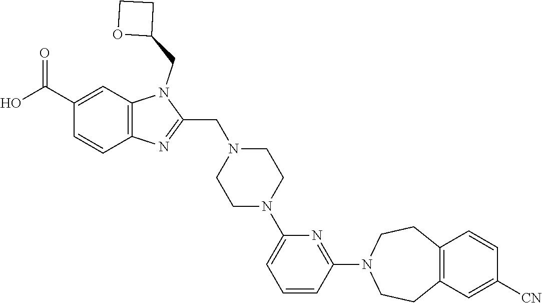 [SMILES: N#Cc1ccc2c(c1)CCN(c1cccc(N3CCN(Cc4nc5ccc(C(=O)O)cc5n4C[C@@H]4CCO4)CC3)n1)CC2] | (S)-2-((4-(6-(7-cyano-4,5-dihydro-1H- benzo[d]azepin-3(2H)-yl)pyridin-2- yl)piperazin-1-yl)methyl)-1-(oxetan-2- ylmethyl)-1H-benzo[d]imidazole-6- carboxylic acid |
| 3 | 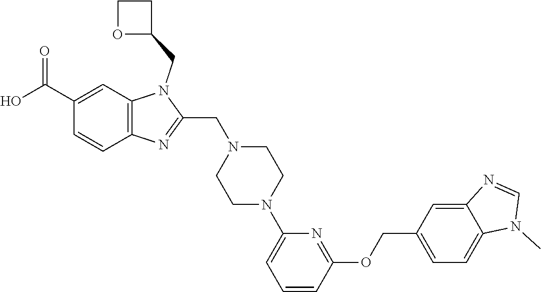 [SMILES: Cn1cnc2cc(COc3cccc(N4CCN(Cc5nc6ccc(C(=O)O)cc6n5C[C@@H]5CCO5)CC4)n3)ccc21] | (S)-2-((4-(6-((1-methyl-1H- benzo[d]imidazol-5-yl)methoxy)pyridin-2- yl)piperazin-1-yl)methyl)-1-(oxetan-2- ylmethyl)-1H-benzo[d]imidazole-6- carboxylic acid |
| 4 | 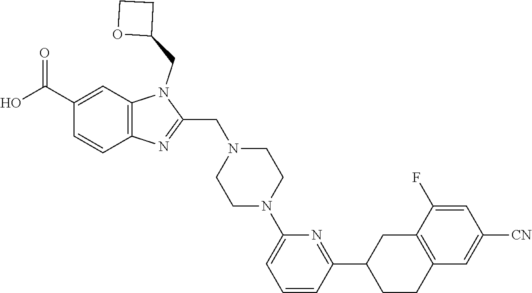 [SMILES: N#Cc1cc(F)c2c(c1)CCC(c1cccc(N3CCN(Cc4nc5ccc(C(=O)O)cc5n4C[C@@H]4CCO4)CC3)n1)C2] | 2-((4-(6-(6-cyano-8-fluoro-1,2,3,4- tetrahydronaphthalen-2-yl)pyridin-2- yl)piperazin-1-yl)methyl)-1-((S)-oxetan-2- ylmethyl)-1H-benzo[d]imidazole-6- carboxylic acid |
| 5 | 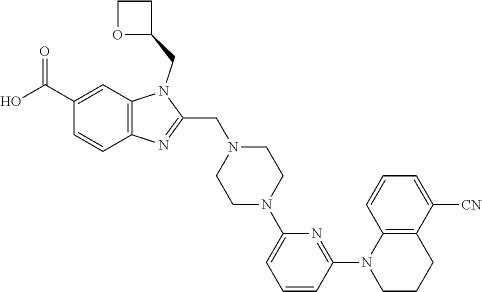 [SMILES: N#Cc1cccc2c1CCCN2c1cccc(N2CCN(Cc3nc4ccc(C(=O)O)cc4n3C[C@@H]3CCO3)CC2)n1] | (S)-2-((4-(6-(5-cyano-3,4- dihydroquinolin-1(2H)-yl)pyridin-2- yl)piperazin-1-yl)methyl)-1-(oxetan-2- ylmethyl)-1H-benzo[d]imidazole-6- carboxylic acid |
| 6-P1 | 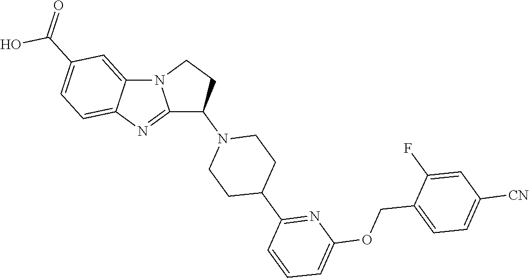 [SMILES: N#Cc1ccc(COc2cccc(C3CCN([C@@H]4CCn5c4nc4ccc(C(=O)O)cc45)CC3)n2)c(F)c1] | (R)-3-(4-(6-((4-cyano-2- fluorobenzyl)oxy)pyridin-2-yl)piperidin-1- yl)-2,3-dihydro-1H-benzo[d]pyrrolo[1,2- a]imidazole-7-carboxylic acid |
| 6-P2 | 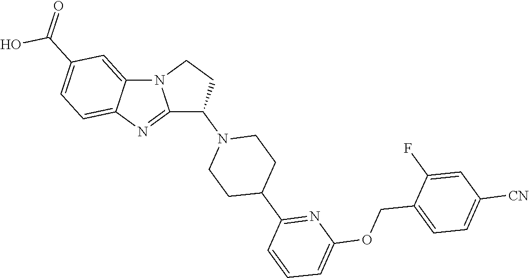 [SMILES: N#Cc1ccc(COc2cccc(C3CCN([C@H]4CCn5c4nc4ccc(C(=O)O)cc45)CC3)n2)c(F)c1] | (S)-3-(4-(6-((4-cyano-2- fluorobenzyl)oxy)pyridin-2-yl)piperidin-1- yl)-2,3-dihydro-1H-benzo[d]pyrrolo[1,2- a]imidazole-7-carboxylic acid |
| 7-P1 | 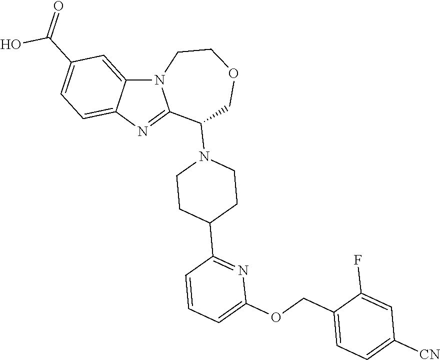 [SMILES: N#Cc1ccc(COc2cccc(C3CCN([C@H]4COCCn5c4nc4ccc(C(=O)O)cc45)CC3)n2)c(F)c1] | (R)-5-(4-(6-((4-cyano-2- fluorobenzyl)oxy)pyridin-2-yl)piperidin-1- yl)-1,2,4,5- tetrahydrobenzo[4,5]imidazo[1,2- d][1,4]oxazepine-9-carboxylic acid |
| 7-P2 | 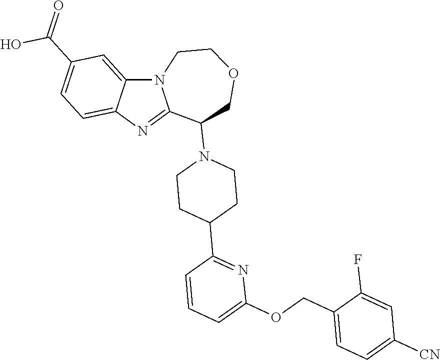 [SMILES: N#Cc1ccc(COc2cccc(C3CCN([C@@H]4COCCn5c4nc4ccc(C(=O)O)cc45)CC3)n2)c(F)c1] | (S)-5-(4-(6-((4-cyano-2- fluorobenzyl)oxy)pyridin-2-yl)piperidin-1- yl)-1,2,4,5- tetrahydrobenzo[4,5]imidazo[1,2- d][1,4]oxazepine-9-carboxylic acid |
| 8-P1 | 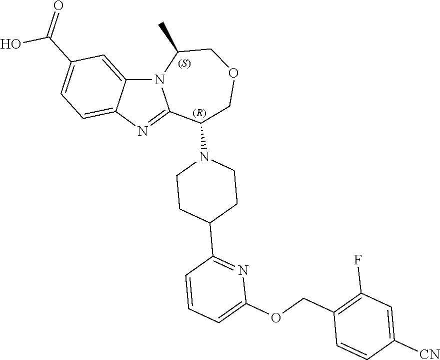 | (1S,5R)-5-(4-(6-((4-cyano-2- fluorobenzyl)oxy)pyridin-2-yl)piperidin-1- yl)-1-methyl-1,2,4,5- tetrahydrobenzo[4,5]imidazo[1,2- d][1,4]oxazepine-9-carboxylic acid |
| 8-P2 | 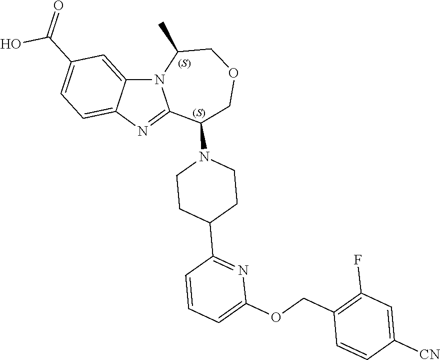 | (1S,5S)-5-(4-(6-((4-cyano-2- fluorobenzyl)oxy)pyridin-2-yl)piperidin-1- yl)-1-methyl-1,2,4,5- tetrahydrobenzo[4,5]imidazo[1,2- d][1,4]oxazepine-9-carboxylic acid |
| 9 | 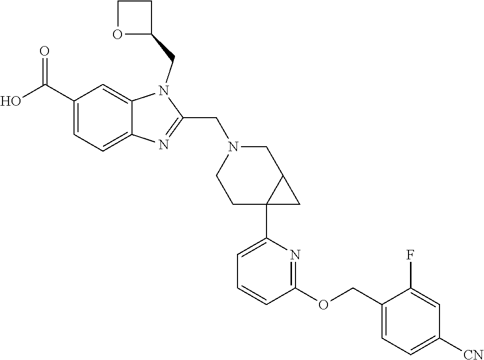 [SMILES: N#Cc1ccc(COc2cccc(C34CCN(Cc5nc6ccc(C(=O)O)cc6n5C[C@@H]5CCO5)CC3C4)n2)c(F)c1] | 2-((6-(6-((4-cyano-2- fluorobenzyl)oxy)pyridin-2-yl)-3- azabicyclo[4.1.0]heptan-3-yl)methyl)-1- ((S)-oxetan-2-ylmethyl)-1H- benzo[d]imidazole-6-carboxylic acid |
| 9-P1 | 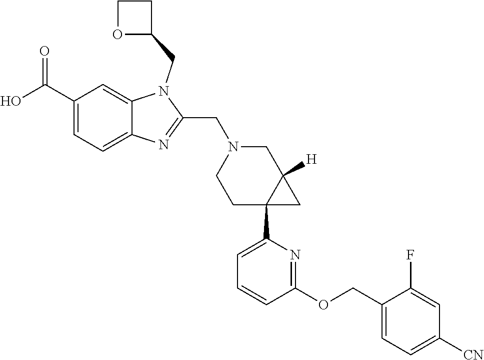 [SMILES: N#Cc1ccc(COc2cccc([C@@]34CCN(Cc5nc6ccc(C(=O)O)cc6n5C[C@@H]5CCO5)C[C@@H]3C4)n2)c(F)c1] | 2-(((1R,6S)-6-(6-((4-cyano-2- fluorobenzyl)oxy)pyridin-2-yl)-3- azabicyclo[4.1.0]heptan-3-yl)methyl)-1- ((S)-oxetan-2-ylmethyl)-1H- benzo[d]imidazole-6-carboxylic acid |
| 9-P2 | 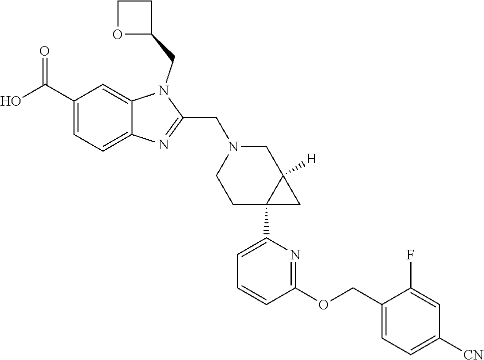 [SMILES: N#Cc1ccc(COc2cccc([C@]34CCN(Cc5nc6ccc(C(=O)O)cc6n5C[C@@H]5CCO5)C[C@H]3C4)n2)c(F)c1] | 2-(((1S,6R)-6-(6-((4-cyano-2- fluorobenzyl)oxy)pyridin-2-yl)-3- azabicyclo[4.1.0]heptan-3-yl)methyl)-1- ((S)-oxetan-2-ylmethyl)-1H- benzo[d]imidazole-6-carboxylic acid |
| 10-P1 | 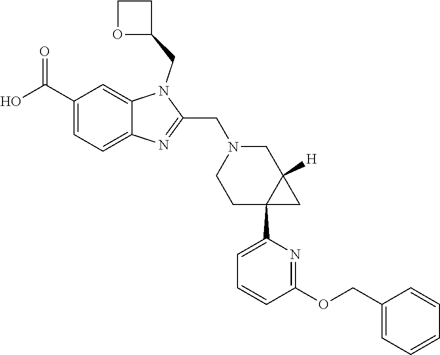 [SMILES: O=C(O)c1ccc2nc(CN3CC[C@@]4(c5cccc(OCc6ccccc6)n5)C[C@H]4C3)n(C[C@@H]3CCO3)c2c1] | 2-(((1R,6S)-6-(6-(benzyloxy)pyridin-2- yl)-3-azabicyclo[4.1.0]heptan-3- yl)methyl)-1-((S)-oxetan-2-ylmethyl)-1H- benzo[d]imidazole-6-carboxylic acid |
| 10-P2 | 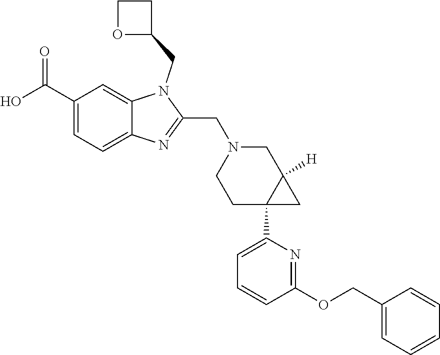 [SMILES: O=C(O)c1ccc2nc(CN3CC[C@]4(c5cccc(OCc6ccccc6)n5)C[C@@H]4C3)n(C[C@@H]3CCO3)c2c1] | 2-(((1S,6R)-6-(6-(benzyloxy)pyridin-2- yl)-3-azabicyclo[4.1.0]heptan-3- yl)methyl)-1-((S)-oxetan-2-ylmethyl)-1H- benzo[d]imidazole-6-carboxylic acid |
| 11-P1 | 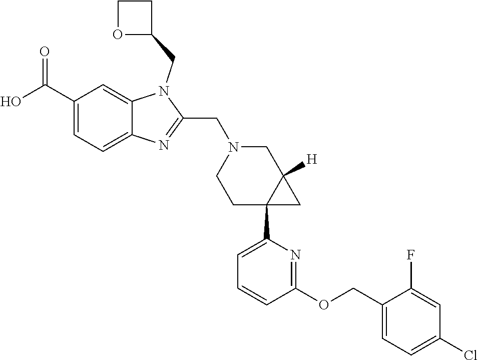 [SMILES: O=C(O)c1ccc2nc(CN3CC[C@@]4(c5cccc(OCc6ccc(Cl)cc6F)n5)C[C@H]4C3)n(C[C@@H]3CCO3)c2c1] | 2-(((1R,6S)-6-(6-((4-chloro-2- fluorobenzyl)oxy)pyridin-2-yl)-3- azabicyclo[4.1.0]heptan-3-yl)methyl)-1- ((S)-oxetan-2-ylmethyl)-1H- benzo[d]imidazole-6-carboxylic acid |
| 11-P2 | 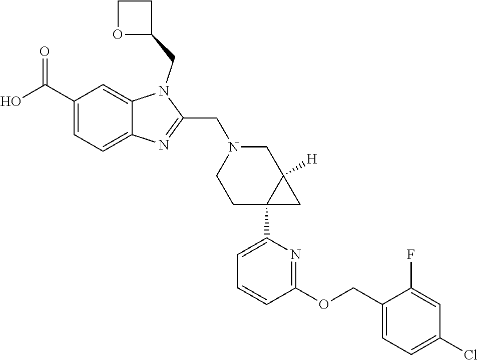 [SMILES: O=C(O)c1ccc2nc(CN3CC[C@]4(c5cccc(OCc6ccc(Cl)cc6F)n5)C[C@@H]4C3)n(C[C@@H]3CCO3)c2c1] | 2-(((1S,6R)-6-(6-((4-chloro-2- fluorobenzyl)oxy)pyridin-2-yl)-3- azabicyclo[4.1.0]heptan-3-yl)methyl)-1- ((S)-oxetan-2-ylmethyl)-1H- benzo[d]imidazole-6-carboxylic acid |
| 12 | 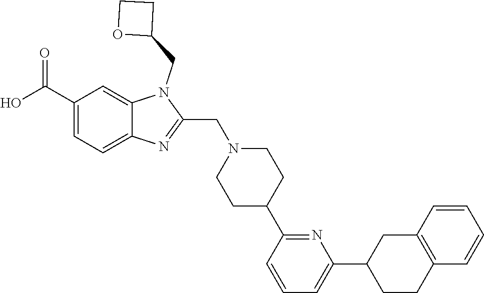 [SMILES: O=C(O)c1ccc2nc(CN3CCC(c4cccc(C5CCc6ccccc6C5)n4)CC3)n(C[C@@H]3CCO3)c2c1] | 1-((S)-oxetan-2-ylmethyl)-2-((4-(6- (1,2,3,4-tetrahydronaphthalen-2- yl)pyridin-2-yl)piperidin-1-yl)methyl)-1H- benzo[d]imidazole-6-carboxylic acid |
| 13 | 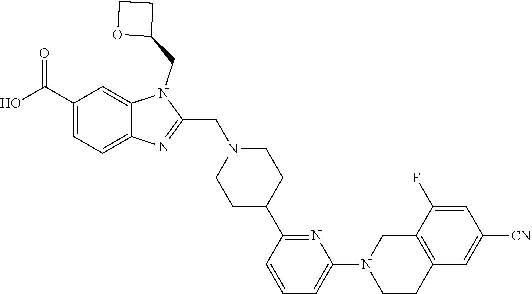 [SMILES: N#Cc1cc(F)c2c(c1)CCN(c1cccc(C3CCN(Cc4nc5ccc(C(=O)O)cc5n4C[C@@H]4CCO4)CC3)n1)C2] | (S)-2-((4-(6-(6-cyano-8-fluoro-3,4- dihydroisoquinolin-2(1H)-yl)pyridin-2- yl)piperidin-1-yl)methyl)-1-(oxetan-2- ylmethyl)-1H-benzo[d]imidazole-6- carboxylic acid |
| 14 | 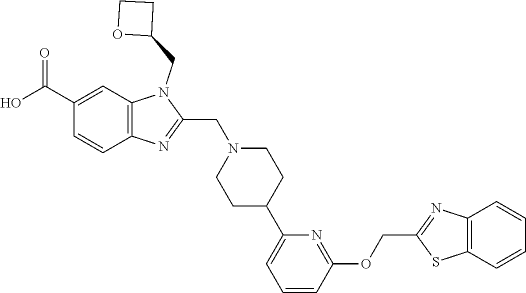 [SMILES: O=C(O)c1ccc2nc(CN3CCC(c4cccc(OCc5nc6ccccc6s5)n4)CC3)n(C[C@@H]3CCO3)c2c1] | (S)-2-((4-(6-(benzo[d]thiazol-2- ylmethoxy)pyridin-2-yl)piperidin-1- yl)methyl)-1-(oxetan-2-ylmethyl)-1H- benzo[d]imidazole-6-carboxylic acid |
| 15 | 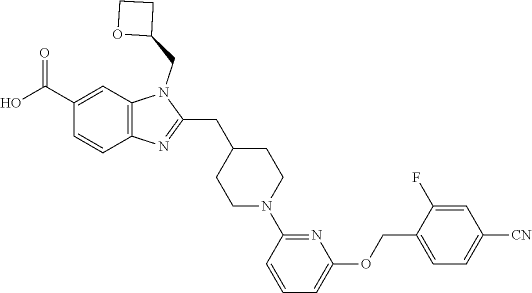 [SMILES: N#Cc1ccc(COc2cccc(N3CCC(Cc4nc5ccc(C(=O)O)cc5n4C[C@@H]4CCO4)CC3)n2)c(F)c1] | (S)-2-((1-(6-((4-cyano-2- fluorobenzyl)oxy)pyridin-2-yl)piperidin-4- yl)methyl)-1-(oxetan-2-ylmethyl)-1H- benzo[d]imidazole-6-carboxylic acid |
| 16 | 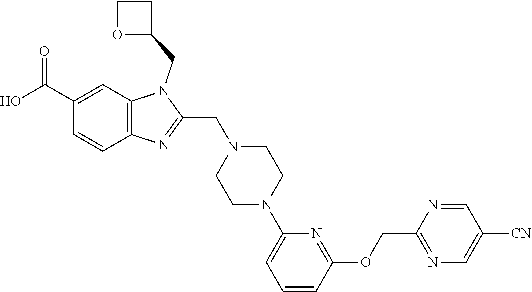 [SMILES: N#Cc1cnc(COc2cccc(N3CCN(Cc4nc5ccc(C(=O)O)cc5n4C[C@@H]4CCO4)CC3)n2)nc1] | (S)-2-((4-(6-((5-cyanopyrimidin-2- yl)methoxy)pyridin-2-yl)piperazin-1- yl)methyl)-1-(oxetan-2-ylmethyl)-1H- benzo[d]imidazole-6-carboxylic acid |
| 17 | 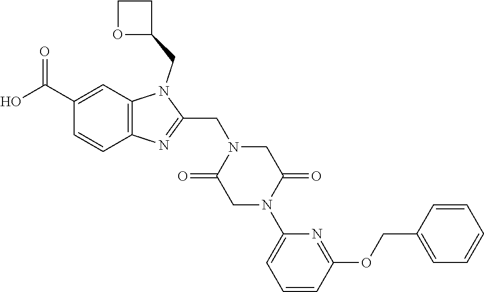 [SMILES: O=C(O)c1ccc2nc(CN3CC(=O)N(c4cccc(OCc5ccccc5)n4)CC3=O)n(C[C@@H]3CCO3)c2c1] | S)-2-((4-(6-(benzyloxy)pyridin-2-yl)-2,5- dioxopiperazin-1-yl)methyl)-1-(oxetan-2- ylmethyl)-1H-benzo[d]imidazole-6- carboxylic acid |
| 18 | 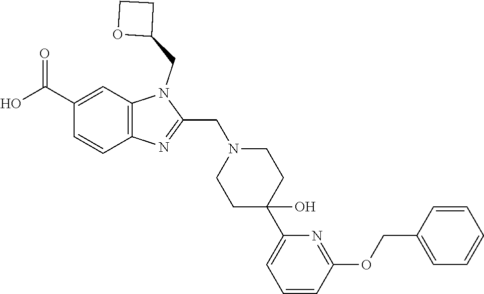 [SMILES: O=C(O)c1ccc2nc(CN3CCC(O)(c4cccc(OCc5ccccc5)n4)CC3)n(C[C@@H]3CCO3)c2c1] | (S)-2-((4-(6-(benzyloxy)pyridin-2-yl)-4- hydroxypiperidin-1-yl)methyl)-1-(oxetan- 2-ylmethyl)-1H-benzo[d]imidazole-6- carboxylic acid |
| 19 | 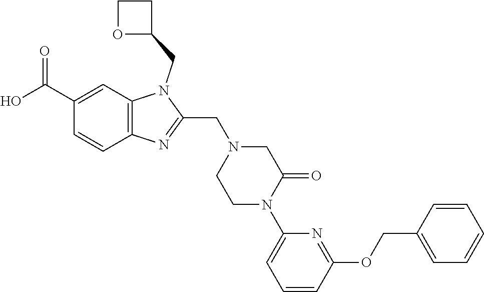 [SMILES: O=C(O)c1ccc2nc(CN3CCN(c4cccc(OCc5ccccc5)n4)C(=O)C3)n(C[C@@H]3CCO3)c2c1] | (S)-2-((4-(6-(benzyloxy)pyridin-2-yl)-3- oxopiperazin-1-yl)methyl)-1-(oxetan-2- ylmethyl)-1H-benzo[d]imidazole-6- carboxylic acid |
| 20-P1 | 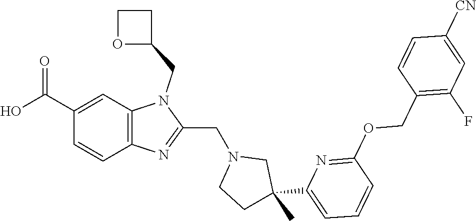 [SMILES: C[C@@]1(c2cccc(OCc3ccc(C#N)cc3F)n2)CCN(Cc2nc3ccc(C(=O)O)cc3n2C[C@@H]2CCO2)C1] | 2-(((R)-3-(6-((4-cyano-2- fluorobenzyl)oxy)pyridin-2-yl)-3- methylpyrrolidin-1-yl)methyl)-1-((S)- oxetan-2-ylmethyl)-1H- benzo[d]imidazole-6-carboxylic acid |
| 20-P2 | 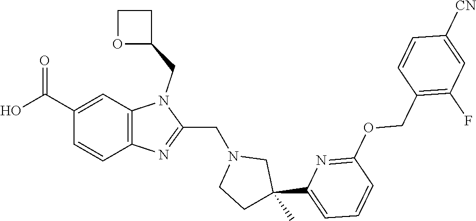 [SMILES: C[C@]1(c2cccc(OCc3ccc(C#N)cc3F)n2)CCN(Cc2nc3ccc(C(=O)O)cc3n2C[C@@H]2CCO2)C1] | 2-(((S)-3-(6-((4-cyano-2- fluorobenzyl)oxy)pyridin-2-yl)-3- methylpyrrolidin-1-yl)methyl)-1-((S)- oxetan-2-ylmethyl)-1H- benzo[d]imidazole-6-carboxylic acid |
| 21 | 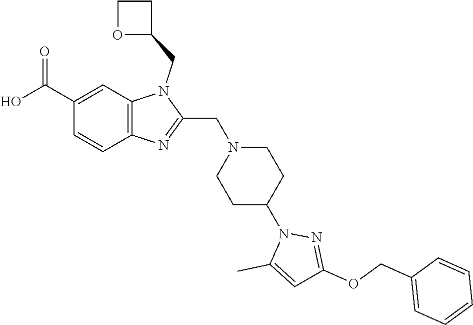 [SMILES: Cc1cc(OCc2ccccc2)nn1C1CCN(Cc2nc3ccc(C(=O)O)cc3n2C[C@@H]2CCO2)CC1] | (S)-2-((4-(3-(benzyloxy)-5-methyl-1H- pyrazol-1-yl)piperidin-1-yl)methyl)-1- (oxetan-2-ylmethyl)-1H- benzo[d]imidazole-6-carboxylic acid |
| 22 | 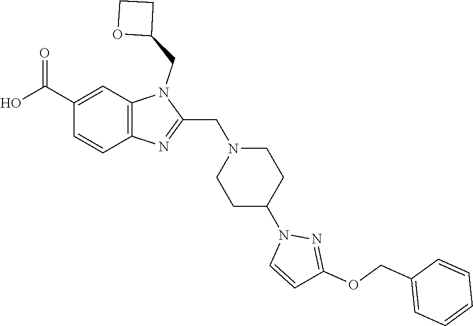 [SMILES: O=C(O)c1ccc2nc(CN3CCC(n4ccc(OCc5ccccc5)n4)CC3)n(C[C@@H]3CCO3)c2c1] | (S)-2-((4-(3-(benzyloxy)-1H-pyrazol-1- yl)piperidin-1-yl)methyl)-1-(oxetan-2- ylmethyl)-1H-benzo[d]imidazole-6- carboxylic |
| 23 | 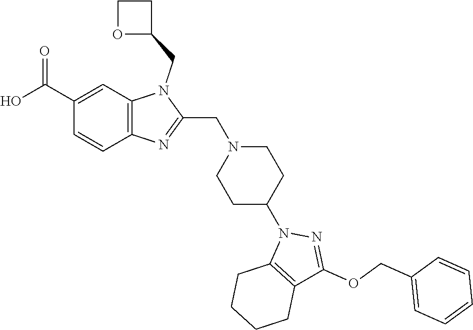 [SMILES: O=C(O)c1ccc2nc(CN3CCC(n4nc(OCc5ccccc5)c5c4CCCC5)CC3)n(C[C@@H]3CCO3)c2c1] | (S)-2-((4-(3-(benzyloxy)-4,5,6,7- tetrahydro-1H-indazol-1-yl)piperidin-1- yl)methyl)-1-(oxetan-2-ylmethyl)-1H- benzo[d]imidazole-6-carboxylic acid |
| 24 | 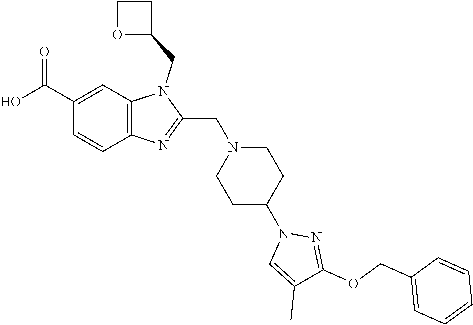 [SMILES: Cc1cn(C2CCN(Cc3nc4ccc(C(=O)O)cc4n3C[C@@H]3CCO3)CC2)nc1OCc1ccccc1] | (S)-2-((4-(3-(benzyloxy)-4-methyl-1H- pyrazol-1-yl)piperidin-1-yl)methyl)-1- (oxetan-2-ylmethyl)-1H- benzo[d]imidazole-6-carboxylic acid |
| 25 | 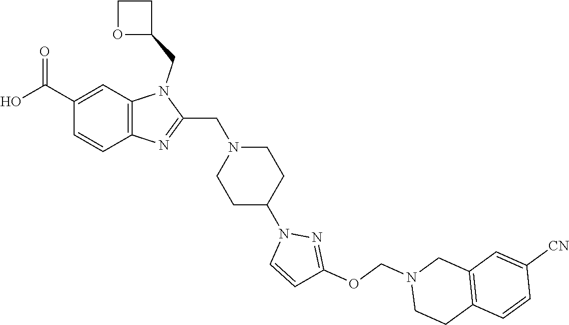 [SMILES: N#Cc1ccc2c(c1)CN(COc1ccn(C3CCN(Cc4nc5ccc(C(=O)O)cc5n4C[C@@H]4CCO4)CC3)n1)CC2] | (S)-2-((4-(3-(7-cyano-3,4- dihydroisoquinolin-2(1H)-yl)-1H-pyrazol- 1-yl)piperidin-1-yl)methyl)-1-(oxetan-2- ylmethyl)-1H-benzo[d]imidazole-6- carboxylic acid |
| 26-P1 | 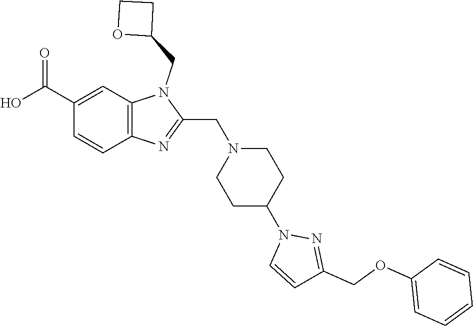 [SMILES: O=C(O)c1ccc2nc(CN3CCC(n4ccc(COc5ccccc5)n4)CC3)n(C[C@@H]3CCO3)c2c1] | (S)-1-(oxetan-2-ylmethyl)-2-((4-(3- (phenoxymethyl)-1H-pyrazol-1- yl)piperidin-1-yl)methyl)-1H- benzo[d]imidazole-6-carboxylic acid |
| 26-P2 | 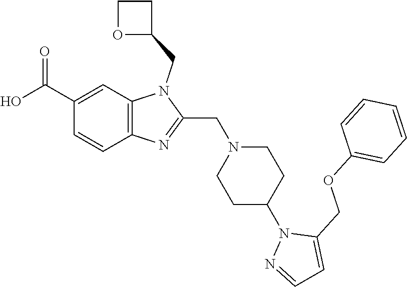 [SMILES: O=C(O)c1ccc2nc(CN3CCC(n4nccc4COc4ccccc4)CC3)n(C[C@@H]3CCO3)c2c1] | (S)-1-(oxetan-2-ylmethyl)-2-((4-(5- (phenoxymethyl)-1H-pyrazol-1- yl)piperidin-1-yl)methyl)-1H- benzo[d]imidazole-6-carboxylic acid |
| 27 | 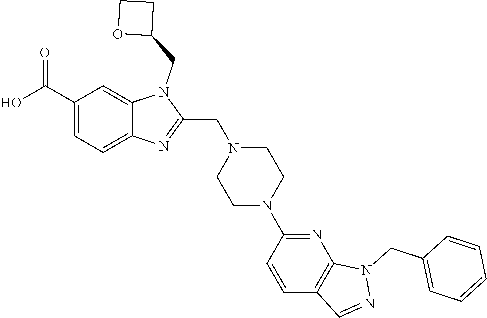 [SMILES: O=C(O)c1ccc2nc(CN3CCN(c4ccc5cnn(Cc6ccccc6)c5n4)CC3)n(C[C@@H]3CCO3)c2c1] | (S)-2-((4-(1-benzyl-1H-pyrazolo[3,4- b]pyridin-6-yl)piperazin-1-yl)methyl)-1- (oxetan-2-ylmethyl)-1H- benzo[d]imidazole-6-carboxylic |
| 28 | 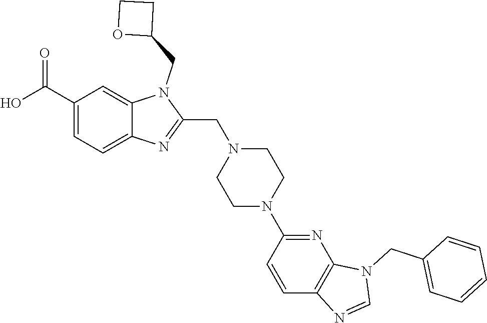 [SMILES: O=C(O)c1ccc2nc(CN3CCN(c4ccc5ncn(Cc6ccccc6)c5n4)CC3)n(C[C@@H]3CCO3)c2c1] | (S)-2-((4-(3-benzyl-3H-imidazo[4,5- b]pyridin-5-yl)piperazin-1-yl)methyl)-1- (oxetan-2-ylmethyl)-1H- benzo[d]imidazole-6-carboxylic acid |
| 29 | 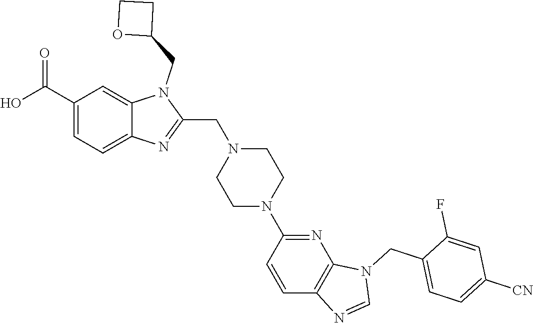 [SMILES: N#Cc1ccc(Cn2cnc3ccc(N4CCN(Cc5nc6ccc(C(=O)O)cc6n5C[C@@H]5CCO5)CC4)nc32)c(F)c1] | (S)-2-((4-(1-(4-cyano-2-fluorobenzyl)-1H- pyrrolo[2,3-b]pyridin-6-yl)piperazin-1- yl)methyl)-1-(oxetan-2-ylmethyl)-1H- benzo[d]imidazole-6-carboxylic acid |
| 30 | 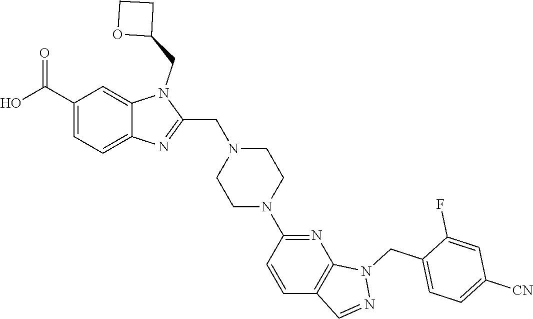 [SMILES: N#Cc1ccc(Cn2ncc3ccc(N4CCN(Cc5nc6ccc(C(=O)O)cc6n5C[C@@H]5CCO5)CC4)nc32)c(F)c1] | (S)-2-((4-(1-(4-cyano-2-fluorobenzyl)-1H- pyrazolo[3,4-b]pyridin-6-yl)piperazin-1- yl)methyl)-1-(oxetan-2-ylmethyl)-1H- benzo[d]imidazole-6-carboxylic acid |
| 31 | 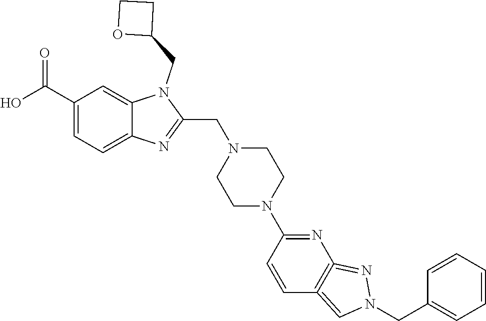 | (S)-2-((4-(2-benzyl-2H-pyrazolo[3,4- b]pyridin-6-yl)piperazin-1-yl)methyl)-1- (oxetan-2-ylmethyl)-1H- benzo[d]imidazole-6-carboxylic acid |
| 32 | 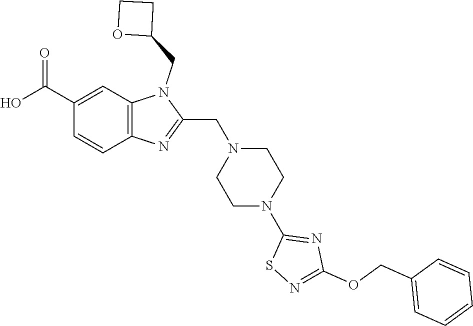 [SMILES: O=C(O)c1ccc2nc(CN3CCN(c4nc(OCc5ccccc5)ns4)CC3)n(C[C@@H]3CCO3)c2c1] | (S)-2-((4-(3-(benzyloxy)-1,2,4-thiadiazol- 5-yl)piperazin-1-yl)methyl)-1-(oxetan-2- ylmethyl)-1H-benzo[d]imidazole-6- carboxylic acid |
| 33 | 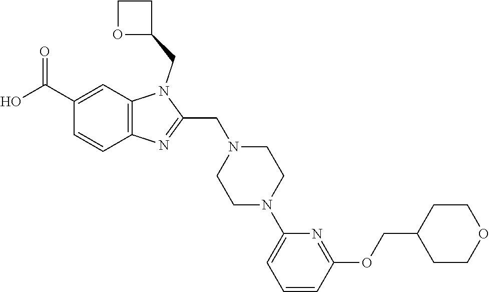 [SMILES: O=C(O)c1ccc2nc(CN3CCN(c4cccc(OCC5CCOCC5)n4)CC3)n(C[C@@H]3CCO3)c2c1] | (S)-1-(oxetan-2-ylmethyl)-2-((4-(6- ((tetrahydro-2H-pyran-4- yl)methoxy)pyridin-2-yl)piperazin-1- yl)methyl)-1H-benzo[d]imidazole-6- carboxylic acid |
| 34 | 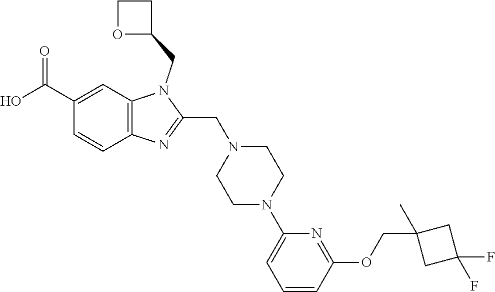 [SMILES: CC1(COc2cccc(N3CCN(Cc4nc5ccc(C(=O)O)cc5n4C[C@@H]4CCO4)CC3)n2)CC(F)(F)C1] | (S)-2-((4-(6-((3,3-difluoro-1- methylcyclobutyl)methoxy)pyridin-2- yl)piperazin-1-yl)methyl)-1-(oxetan-2- ylmethyl)-1H-benzo[d]imidazole-6- carboxylic acid |
| 35 | 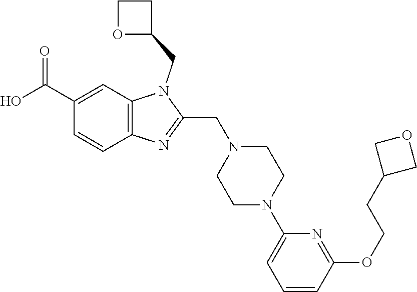 [SMILES: O=C(O)c1ccc2nc(CN3CCN(c4cccc(OCCC5COC5)n4)CC3)n(C[C@@H]3CCO3)c2c1] | (S)-1-(oxetan-2-ylmethyl)-2-((4-(6-(2- (oxetan-3-yl)ethoxy)pyridin-2- yl)piperazin-1-yl)methyl)-1H- benzo[d]imidazole-6-carboxylic acid |
| 36 | 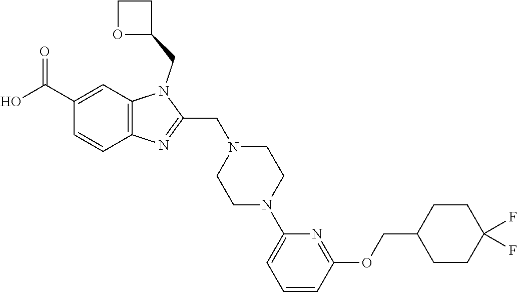 [SMILES: O=C(O)c1ccc2nc(CN3CCN(c4cccc(OCC5CCC(F)(F)CC5)n4)CC3)n(C[C@@H]3CCO3)c2c1] | (S)-2-((4-(6-((4,4- difluorocyclohexyl)methoxy)pyridin-2- yl)piperazin-1-yl)methyl)-1-(oxetan-2- ylmethyl)-1H-benzo[d]imidazole-6- carboxylic acid |
| 37 | 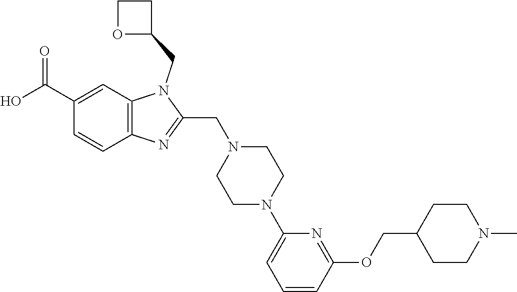 [SMILES: CN1CCC(COc2cccc(N3CCN(Cc4nc5ccc(C(=O)O)cc5n4C[C@@H]4CCO4)CC3)n2)CC1] | (S)-2-((4-(6-((1-methylpiperidin-4- yl)methoxy)pyridin-2-yl)piperazin-1- yl)methyl)-1-(oxetan-2-ylmethyl)-1H- benzo[d]imidazole-6-carboxylic acid |
| 38 | 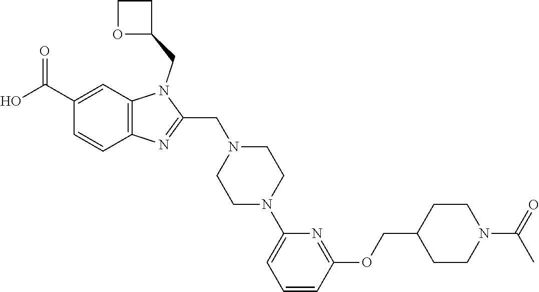 [SMILES: CC(=O)N1CCC(COc2cccc(N3CCN(Cc4nc5ccc(C(=O)O)cc5n4C[C@@H]4CCO4)CC3)n2)CC1] | (S)-2-((4-(6-((1-acetylpiperidin-4- yl)methoxy)pyridin-2-yl)piperazin-1- yl)methyl)-1-(oxetan-2-ylmethyl)-1H- benzo[d]imidazole-6-carboxylic acid |
| 39 | 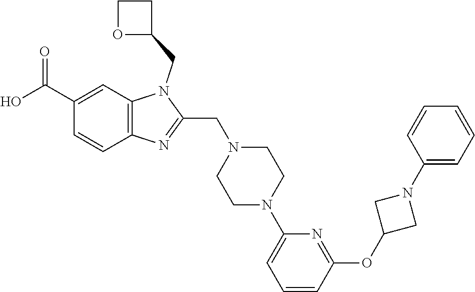 [SMILES: O=C(O)c1ccc2nc(CN3CCN(c4cccc(OC5CN(c6ccccc6)C5)n4)CC3)n(C[C@@H]3CCO3)c2c1] | (S)-1-(oxetan-2-ylmethyl)-2-((4-(6-((1- phenylazetidin-3-yl)oxy)pyridin-2- yl)piperazin-1-yl)methyl)-1H- benzo[d]imidazole-6-carboxylic acid |
| 40 | 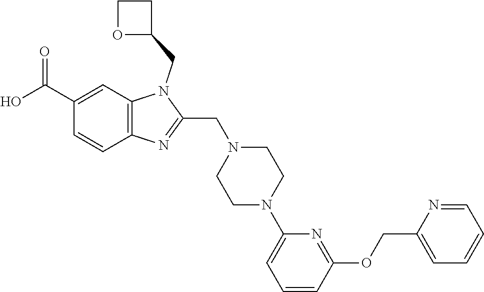 [SMILES: O=C(O)c1ccc2nc(CN3CCN(c4cccc(OCc5ccccn5)n4)CC3)n(C[C@@H]3CCO3)c2c1] | (S)-1-(oxetan-2-ylmethyl)-2-((4-(6- (pyridin-2-ylmethoxy)pyridin-2- yl)piperazin-1-yl)methyl)-1H- benzo[d]imidazole-6-carboxylic acid |
| 41 | 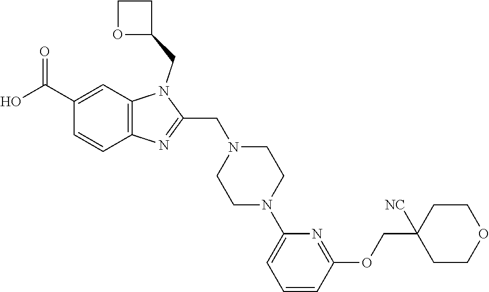 [SMILES: N#CC1(COc2cccc(N3CCN(Cc4nc5ccc(C(=O)O)cc5n4C[C@@H]4CCO4)CC3)n2)CCOCC1] | (S)-2-((4-(6-((4-cyanotetrahydro-2H- pyran-4-yl)methoxy)pyridin-2- yl)piperazin-1-yl)methyl)-1-(oxetan-2- ylmethyl)-1H-benzo[d]imidazole-6- carboxylic acid |
| 42 | 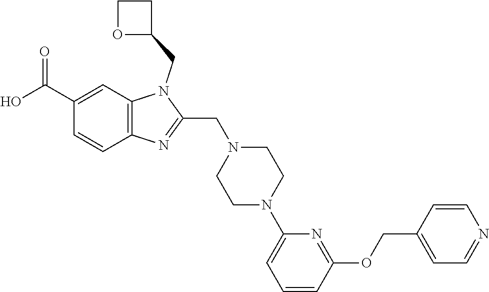 [SMILES: O=C(O)c1ccc2nc(CN3CCN(c4cccc(OCc5ccncc5)n4)CC3)n(C[C@@H]3CCO3)c2c1] | (S)-1-(oxetan-2-ylmethyl)-2-((4-(6- (pyridin-4-ylmethoxy)pyridin-2- yl)piperazin-1-yl)methyl)-1H- benzo[d]imidazole-6-carboxylic acid |
| 43 | 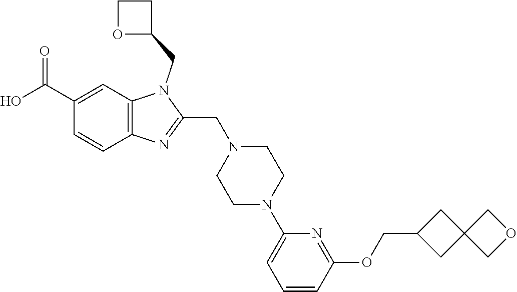 [SMILES: O=C(O)c1ccc2nc(CN3CCN(c4cccc(OCC5CC6(COC6)C5)n4)CC3)n(C[C@@H]3CCO3)c2c1] | (S)-2-((4-(6-((2-oxaspiro[3.3]heptan-6- yl)methoxy)pyridin-2-yl)piperazin-1- yl)methyl)-1-(oxetan-2-ylmethyl)-1H- benzo[d]imidazole-6-carboxylic acid |
| 44 | 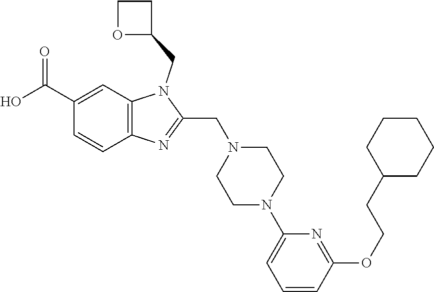 [SMILES: O=C(O)c1ccc2nc(CN3CCN(c4cccc(OCCC5CCCCC5)n4)CC3)n(C[C@@H]3CCO3)c2c1] | (S)-2-((4-(6-(2-cyclohexylethoxy)pyridin- 2-yl)piperazin-1-yl)methyl)-1-(oxetan-2- ylmethyl)-1H-benzo[d]imidazole-6- carboxylic acid |
| 45 | 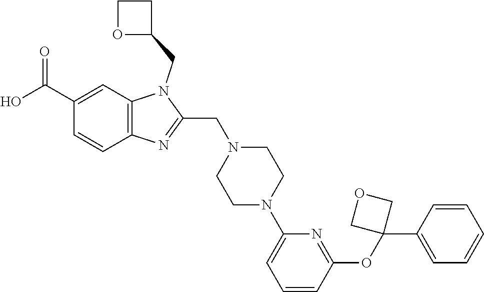 [SMILES: O=C(O)c1ccc2nc(CN3CCN(c4cccc(OC5(c6ccccc6)COC5)n4)CC3)n(C[C@@H]3CCO3)c2c1] | (S)-1-(oxetan-2-ylmethyl)-2-((4-(6-((3- phenyloxetan-3-yl)oxy)pyridin-2- yl)piperazin-1-yl)methyl)-1H- benzo[d]imidazole-6-carboxylic acid |
| 46 | 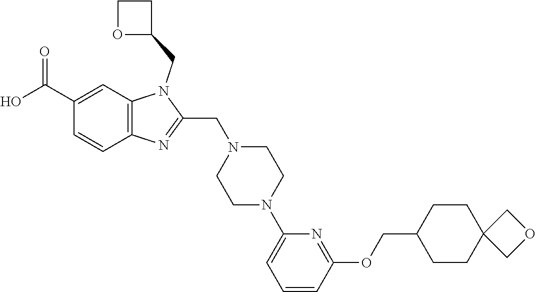 [SMILES: O=C(O)c1ccc2nc(CN3CCN(c4cccc(OCC5CCC6(CC5)COC6)n4)CC3)n(C[C@@H]3CCO3)c2c1] | (S)-2-((4-(6-((2-oxaspiro[3.5]nonan-7- yl)methoxy)pyridin-2-yl)piperazin-1- yl)methyl)-1-(oxetan-2-ylmethyl)-1H- benzo[d]imidazole-6-carboxylic acid |
| 47 | 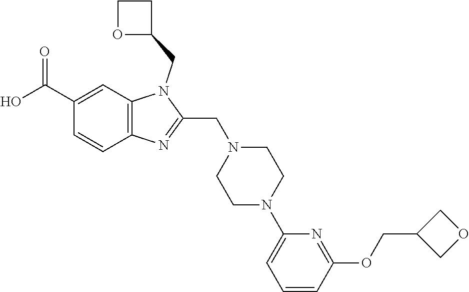 [SMILES: O=C(O)c1ccc2nc(CN3CCN(c4cccc(OCC5COC5)n4)CC3)n(C[C@@H]3CCO3)c2c1] | (S)-1-(oxetan-2-ylmethyl)-2-((4-(6- (oxetan-3-ylmethoxy)pyridin-2- yl)piperazin-1-yl)methyl)-1H- benzo[d]imidazole-6-carboxylic acid |
| 48 | 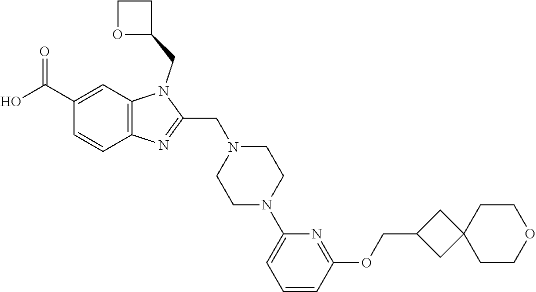 [SMILES: O=C(O)c1ccc2nc(CN3CCN(c4cccc(OCC5CC6(CCOCC6)C5)n4)CC3)n(C[C@@H]3CCO3)c2c1] | (S)-2-((4-(6-((7-oxaspiro[3.5]nonan-2- yl)methoxy)pyridin-2-yl)piperazin-1- yl)methyl)-1-(oxetan-2-ylmethyl)-1H- benzo[d]imidazole-6-carboxylic acid |
| 49 | 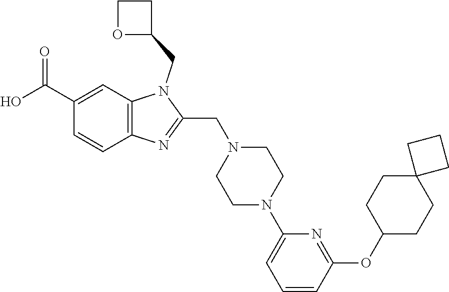 [SMILES: O=C(O)c1ccc2nc(CN3CCN(c4cccc(OC5CCC6(CCC6)CC5)n4)CC3)n(C[C@@H]3CCO3)c2c1] | (S)-1-(oxetan-2-ylmethyl)-2-((4-(6- (spiro[3.5]nonan-7-yloxy)pyridin-2- yl)piperazin-1-yl)methyl)-1H- benzo[d]imidazole-6-carboxylic acid |
| 50 | 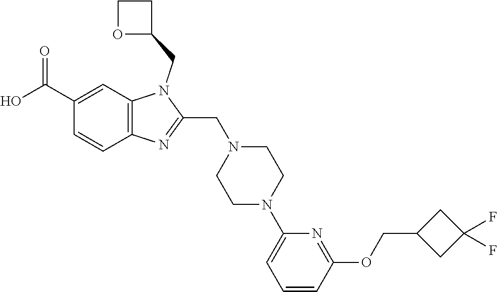 [SMILES: O=C(O)c1ccc2nc(CN3CCN(c4cccc(OCC5CC(F)(F)C5)n4)CC3)n(C[C@@H]3CCO3)c2c1] | (S)-2-((4-(6-((3,3- difluorocyclobutyl)methoxy)pyridin-2- yl)piperazin-1-yl)methyl)-1-(oxetan-2- ylmethyl)-1H-benzo[d]imidazole-6- carboxylic acid |
| 51 | 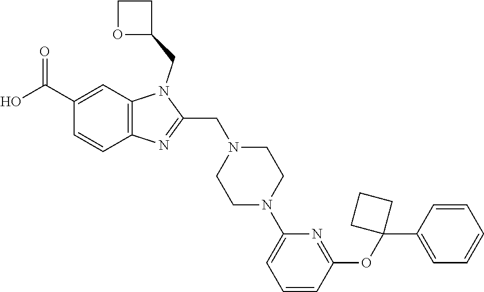 [SMILES: O=C(O)c1ccc2nc(CN3CCN(c4cccc(OC5(c6ccccc6)CCC5)n4)CC3)n(C[C@@H]3CCO3)c2c1] | (S)-1-(oxetan-2-ylmethyl)-2-((4-(6-(1- phenylcyclobutoxy)pyridin-2-yl)piperazin- 1-yl)methyl)-1H-benzo[d]imidazole-6- carboxylic acid |
| 52 | 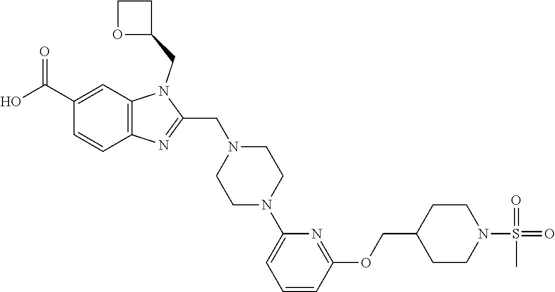 [SMILES: CS(=O)(=O)N1CCC(COc2cccc(N3CCN(Cc4nc5ccc(C(=O)O)cc5n4C[C@@H]4CCO4)CC3)n2)CC1] | (S)-2-((4-(6-((1-(methylsulfonyl)piperidin- 4-yl)methoxy)pyridin-2-yl)piperazin-1- yl)methyl)-1-(oxetan-2-ylmethyl)-1H- benzo[d]imidazole-6-carboxylic acid |
| 53 | 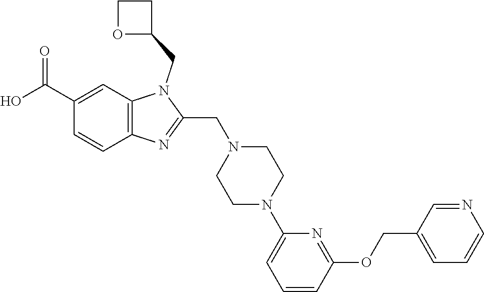 [SMILES: O=C(O)c1ccc2nc(CN3CCN(c4cccc(OCc5cccnc5)n4)CC3)n(C[C@@H]3CCO3)c2c1] | (S)-1-(oxetan-2-ylmethyl)-2-((4-(6- (pyridin-3-ylmethoxy)pyridin-2- yl)piperazin-1-yl)methyl)-1H- benzo[d]imidazole-6-carboxylic acid |
| 54 | 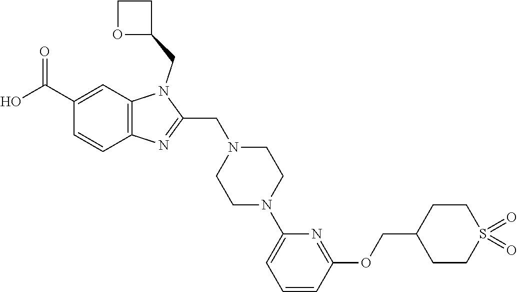 [SMILES: O=C(O)c1ccc2nc(CN3CCN(c4cccc(OCC5CCS(=O)(=O)CC5)n4)CC3)n(C[C@@H]3CCO3)c2c1] | (S)-2-((4-(6-((1,1-dioxidotetrahydro-2H- thiopyran-4-yl)methoxy)pyridin-2- yl)piperazin-1-yl)methyl)-1-(oxetan-2- ylmethyl)-1H-benzo[d]imidazole-6- carboxylic acid |
| 55 | 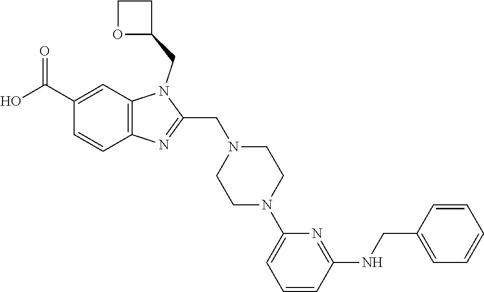 [SMILES: O=C(O)c1ccc2nc(CN3CCN(c4cccc(NCc5ccccc5)n4)CC3)n(C[C@@H]3CCO3)c2c1] | (S)-2-((4-(6-(benzylamino)pyridin-2- yl)piperazin-1-yl)methyl)-1-(oxetan-2- ylmethyl)-1H-benzo[d]imidazole-6- carboxylic acid |
| 56 | 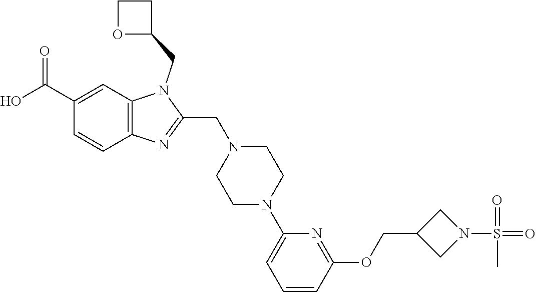 [SMILES: CS(=O)(=O)N1CC(COc2cccc(N3CCN(Cc4nc5ccc(C(=O)O)cc5n4C[C@@H]4CCO4)CC3)n2)C1] | (S)-2-((4-(6-((1-(methylsulfonyl)azetidin- 3-yl)methoxy)pyridin-2-yl)piperazin-1- yl)methyl)-1-(oxetan-2-ylmethyl)-1H- benzo[d]imidazole-6-carboxylic acid |
| 57 | 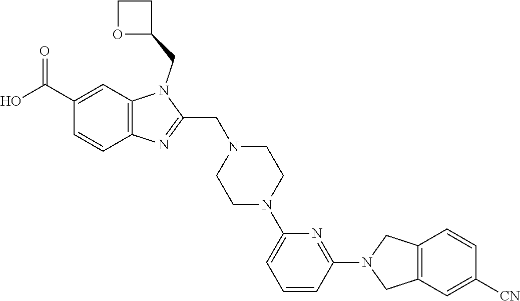 [SMILES: N#Cc1ccc2c(c1)CN(c1cccc(N3CCN(Cc4nc5ccc(C(=O)O)cc5n4C[C@@H]4CCO4)CC3)n1)C2] | (S)-2-((4-(6-(5-cyanoisoindolin-2- yl)pyridin-2-yl)piperazin-1-yl)methyl)-1- (oxetan-2-ylmethyl)-1H- benzo[d]imidazole-6-carboxylic acid |
| 58 | 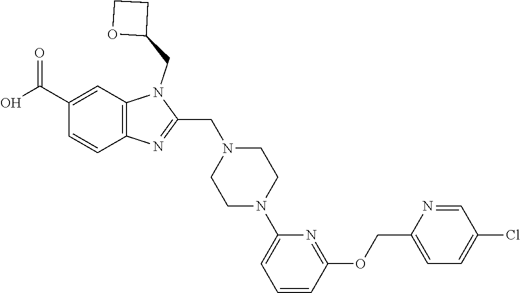 [SMILES: O=C(O)c1ccc2nc(CN3CCN(c4cccc(OCc5ccc(Cl)cn5)n4)CC3)n(C[C@@H]3CCO3)c2c1] | (S)-2-((4-(6-((5-chloropyridin-2- yl)methoxy)pyridin-2-yl)piperazin-1- yl)methyl)-1-(oxetan-2-ylmethyl)-1H- benzo[d]imidazole-6-carboxylic acid |
| 59 | 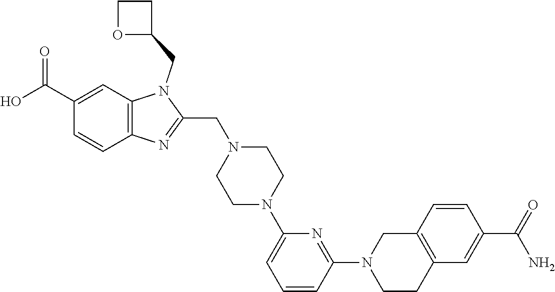 [SMILES: NC(=O)c1ccc2c(c1)CCN(c1cccc(N3CCN(Cc4nc5ccc(C(=O)O)cc5n4C[C@@H]4CCO4)CC3)n1)C2] | (S)-2-((4-(6-(6-carbamoyl-3,4- dihydroisoquinolin-2(1H)-yl)pyridin-2- yl)piperazin-1-yl)methyl)-1-(oxetan-2- ylmethyl)-1H-benzo[d]imidazole-6- carboxylic acid |
| 60 | 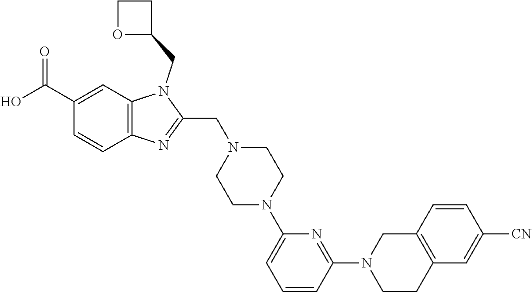 [SMILES: N#Cc1ccc2c(c1)CCN(c1cccc(N3CCN(Cc4nc5ccc(C(=O)O)cc5n4C[C@@H]4CCO4)CC3)n1)C2] | (S)-2-((4-(6-(6-cyano-3,4- dihydroisoquinolin-2(1H)-yl)pyridin-2- yl)piperazin-1-yl)methyl)-1-(oxetan-2- ylmethyl)-1H-benzo[d]imidazole-6- carboxylic acid |
| 61 | 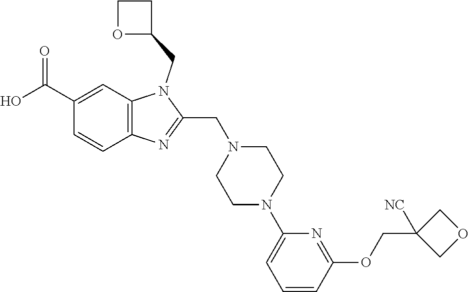 [SMILES: N#CC1(COc2cccc(N3CCN(Cc4nc5ccc(C(=O)O)cc5n4C[C@@H]4CCO4)CC3)n2)COC1] | (S)-2-((4-(6-((3-cyanooxetan-3- yl)methoxy)pyridin-2-yl)piperazin-1- yl)methyl)-1-(oxetan-2-ylmethyl)-1H- benzo[d]imidazole-6-carboxylic acid |
| 62 | 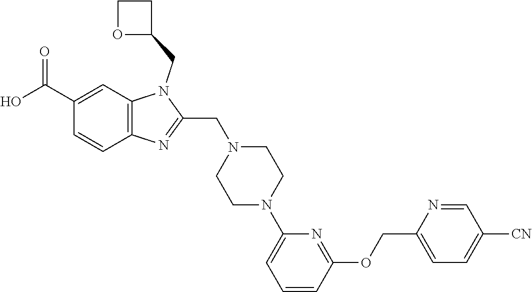 [SMILES: N#Cc1ccc(COc2cccc(N3CCN(Cc4nc5ccc(C(=O)O)cc5n4C[C@@H]4CCO4)CC3)n2)nc1] | (S)-2-((4-(6-((5-cyanopyridin-2- yl)methoxy)pyridin-2-yl)piperazin-1- yl)methyl)-1-(oxetan-2-ylmethyl)-1H- benzo[d]imidazole-6-carboxylic acid |
| 63 | 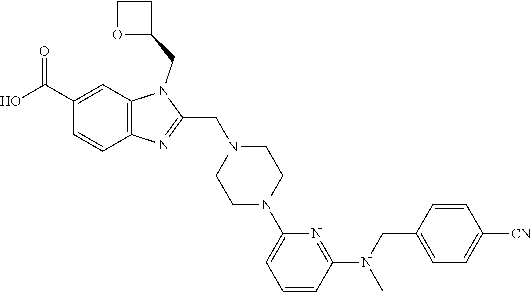 [SMILES: CN(Cc1ccc(C#N)cc1)c1cccc(N2CCN(Cc3nc4ccc(C(=O)O)cc4n3C[C@@H]3CCO3)CC2)n1] | (S)-2-((4-(6-((4- cyanobenzyl)(methyl)amino)pyridin-2- yl)piperazin-1-yl)methyl)-1-(oxetan-2- ylmethyl)-1H-benzo[d]imidazole-6- carboxylic acid |
| 64 | 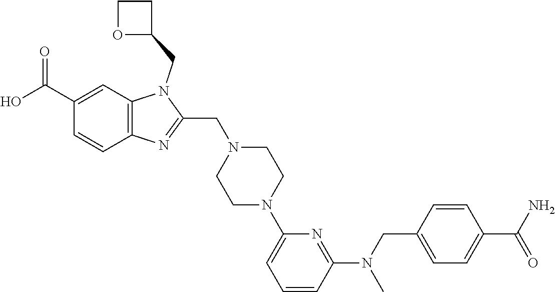 [SMILES: CN(Cc1ccc(C(N)=O)cc1)c1cccc(N2CCN(Cc3nc4ccc(C(=O)O)cc4n3C[C@@H]3CCO3)CC2)n1] | (S)-2-((4-(6-((4- carbamoylbenzyl)(methyl)amino)pyridin- 2-yl)piperazin-1-yl)methyl)-1-(oxetan-2- ylmethyl)-1H-benzo[d]imidazole-6- carboxylic acid |
| 65 | 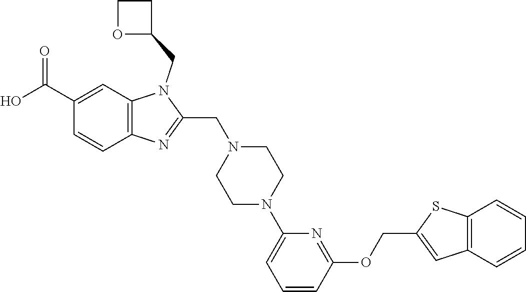 [SMILES: O=C(O)c1ccc2nc(CN3CCN(c4cccc(OCc5cc6ccccc6s5)n4)CC3)n(C[C@@H]3CCO3)c2c1] | (S)-2-((4-(6-(benzo[b]thiophen-2- ylmethoxy)pyridin-2-yl)piperazin-1- yl)methyl)-1-(oxetan-2-ylmethyl)-1H- benzo[d]imidazole-6-carboxylic acid |
| 66 | 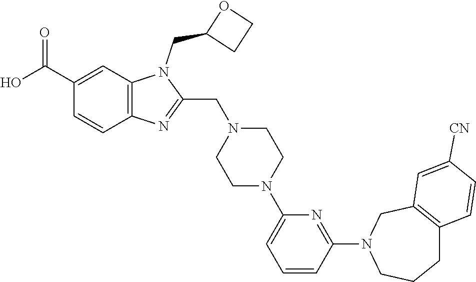 [SMILES: N#Cc1ccc2c(c1)CN(c1cccc(N3CCN(Cc4nc5ccc(C(=O)O)cc5n4C[C@@H]4CCO4)CC3)n1)CCC2] | (S)-2-((4-(6-(8-cyano-1,3,4,5-tetrahydro- 2H-benzo[c]azepin-2-yl)pyridin-2- yl)piperazin-1-yl)methyl)-1-(oxetan-2- ylmethyl)-1H-benzo[d]imidazole-6- carboxylic acid |
| 67 | 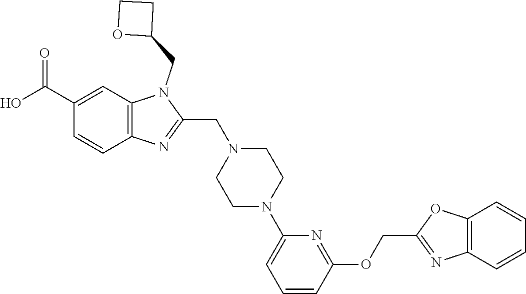 [SMILES: O=C(O)c1ccc2nc(CN3CCN(c4cccc(OCc5nc6ccccc6o5)n4)CC3)n(C[C@@H]3CCO3)c2c1] | (S)-2-((4-(6-(benzo[d]oxazol-2- ylmethoxy)pyridin-2-yl)piperazin-1- yl)methyl)-1-(oxetan-2-ylmethyl)-1H- benzo[d]imidazole-6-carboxylic acid |
| 68 | 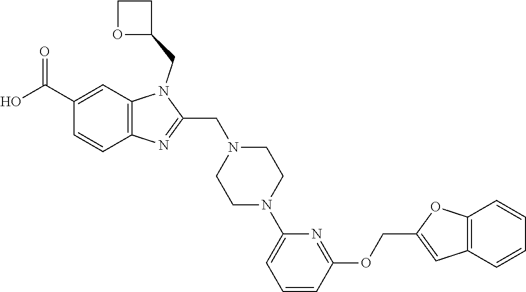 [SMILES: O=C(O)c1ccc2nc(CN3CCN(c4cccc(OCc5cc6ccccc6o5)n4)CC3)n(C[C@@H]3CCO3)c2c1] | (S)-2-((4-(6-(benzofuran-2- ylmethoxy)pyridin-2-yl)piperazin-1- yl)methyl)-1-(oxetan-2-ylmethyl)-1H- benzo[d]imidazole-6-carboxylic acid |
| 69 | 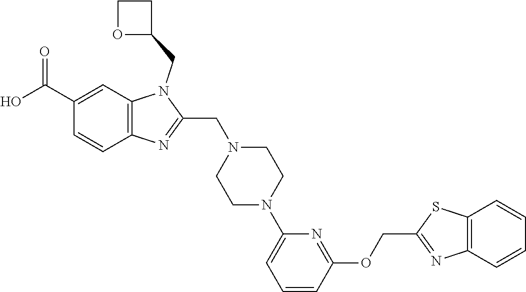 [SMILES: O=C(O)c1ccc2nc(CN3CCN(c4cccc(OCc5nc6ccccc6s5)n4)CC3)n(C[C@@H]3CCO3)c2c1] | (S)-2-((4-(6-(benzo[d]thiazol-2- ylmethoxy)pyridin-2-yl)piperazin-1- yl)methyl)-1-(oxetan-2-ylmethyl)-1H- benzo[d]imidazole-6-carboxylic acid |
| 70 | 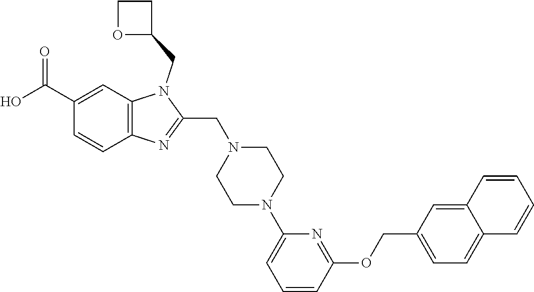 [SMILES: O=C(O)c1ccc2nc(CN3CCN(c4cccc(OCc5ccc6ccccc6c5)n4)CC3)n(C[C@@H]3CCO3)c2c1] | (S)-2-((4-(6-(naphthalen-2- ylmethoxy)pyridin-2-yl)piperazin-1- yl)methyl)-1-(oxetan-2-ylmethyl)-1H- benzo[d]imidazole-6-carboxylic acid |
| 71 | 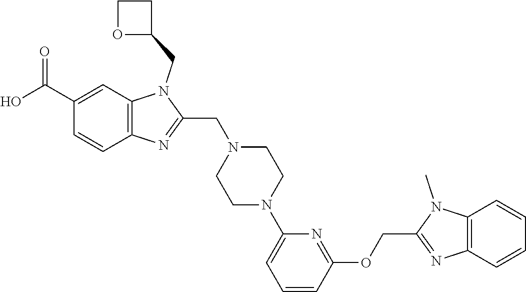 [SMILES: Cn1c(COc2cccc(N3CCN(Cc4nc5ccc(C(=O)O)cc5n4C[C@@H]4CCO4)CC3)n2)nc2ccccc21] | (S)-2-((4-(6-((1-methyl-1H- benzo[d]imidazol-2-yl)methoxy)pyridin-2- yl)piperazin-1-yl)methyl)-1-(oxetan-2- ylmethyl)-1H-benzo[d]imidazole-6- carboxylic acid |
| 72 | 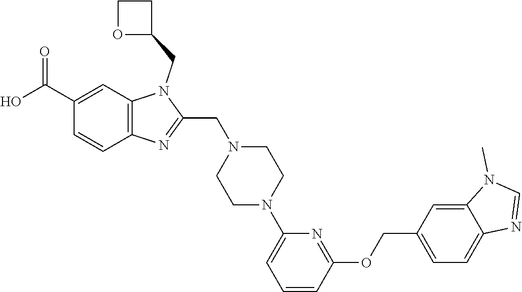 [SMILES: Cn1cnc2ccc(COc3cccc(N4CCN(Cc5nc6ccc(C(=O)O)cc6n5C[C@@H]5CCO5)CC4)n3)cc21] | (S)-2-((4-(6-((1-methyl-1H- benzo[d]imidazol-6-yl)methoxy)pyridin-2- yl)piperazin-1-yl)methyl)-1-(oxetan-2- ylmethyl)-1H-benzo[d]imidazole-6- carboxylic acid |
| 73 | 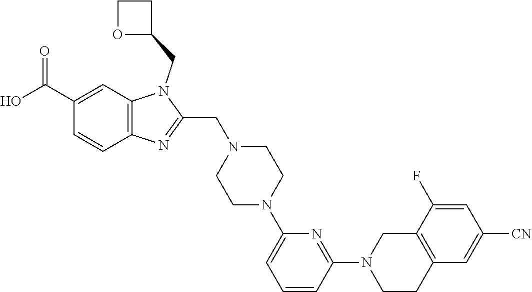 [SMILES: N#Cc1cc(F)c2c(c1)CCN(c1cccc(N3CCN(Cc4nc5ccc(C(=O)O)cc5n4C[C@@H]4CCO4)CC3)n1)C2] | (S)-2-((4-(6-(6-cyano-8-fluoro-3,4- dihydroisoquinolin-2(1H)-yl)pyridin-2- yl)piperazin-1-yl)methyl)-1-(oxetan-2- ylmethyl)-1H-benzo[d]imidazole-6- carboxylic acid |
| 74 | 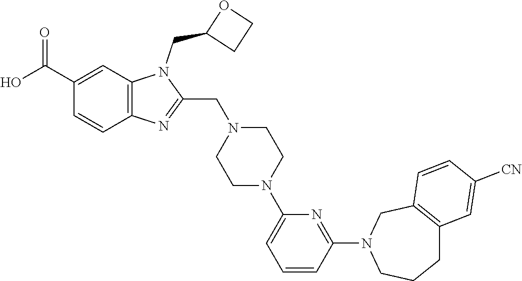 [SMILES: N#Cc1ccc2c(c1)CCCN(c1cccc(N3CCN(Cc4nc5ccc(C(=O)O)cc5n4C[C@@H]4CCO4)CC3)n1)C2] | (S)-2-((4-(6-(7-cyano-1,3,4,5-tetrahydro- 2H-benzo[c]azepin-2-yl)pyridin-2- yl)piperazin-1-yl)methyl)-1-(oxetan-2- ylmethyl)-1H-benzo[d]imidazole-6- carboxylic acid |
| 75 | 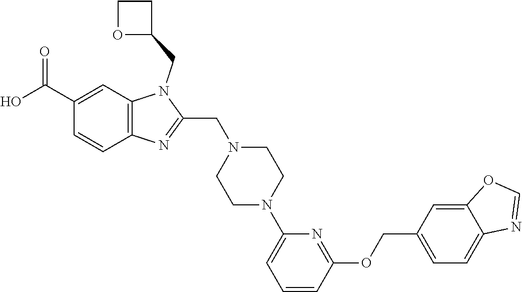 [SMILES: O=C(O)c1ccc2nc(CN3CCN(c4cccc(OCc5ccc6ncoc6c5)n4)CC3)n(C[C@@H]3CCO3)c2c1] | (S)-2-((4-(6-(benzo[d]oxazol-6- ylmethoxy)pyridin-2-yl)piperazin-1- yl)methyl)-1-(oxetan-2-ylmethyl)-1H- benzo[d]imidazole-6-carboxylic acid |
| 76 | 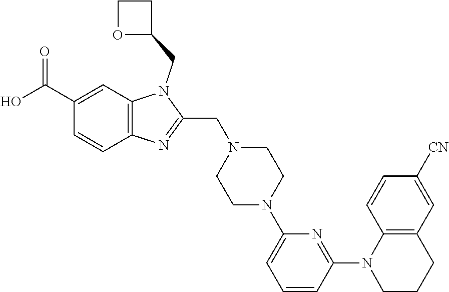 [SMILES: N#Cc1ccc2c(c1)CCCN2c1cccc(N2CCN(Cc3nc4ccc(C(=O)O)cc4n3C[C@@H]3CCO3)CC2)n1] | (S)-2-((4-(6-(6-cyano-3,4- dihydroquinolin-1(2H)-yl)pyridin-2- yl)piperazin-1-yl)methyl)-1-(oxetan-2- ylmethyl)-1H-benzo[d]imidazole-6- carboxylic acid |
| 77 | 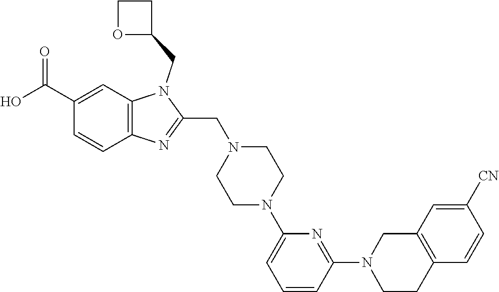 [SMILES: N#Cc1ccc2c(c1)CN(c1cccc(N3CCN(Cc4nc5ccc(C(=O)O)cc5n4C[C@@H]4CCO4)CC3)n1)CC2] | (S)-2-((4-(6-(7-cyano-3,4- dihydroisoquinolin-2(1H)-yl)pyridin-2- yl)piperazin-1-yl)methyl)-1-(oxetan-2- ylmethyl)-1H-benzo[d]imidazole-6- carboxylic acid |
| 78 | 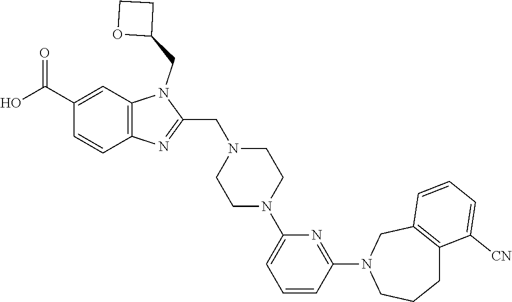 [SMILES: N#Cc1cccc2c1CCCN(c1cccc(N3CCN(Cc4nc5ccc(C(=O)O)cc5n4C[C@@H]4CCO4)CC3)n1)C2] | (S)-2-((4-(6-(6-cyano-1,3,4,5-tetrahydro- 2H-benzo[c]azepin-2-yl)pyridin-2- yl)piperazin-1-yl)methyl)-1-(oxetan-2- ylmethyl)-1H-benzo[d]imidazole-6- carboxylic acid |
| 79 | 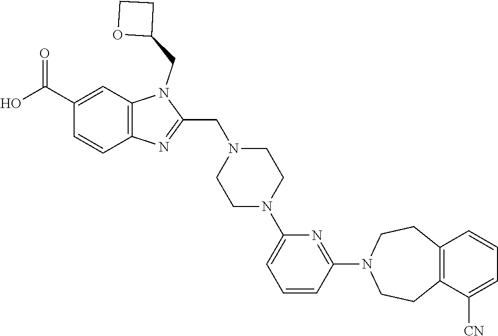 [SMILES: N#Cc1cccc2c1CCN(c1cccc(N3CCN(Cc4nc5ccc(C(=O)O)cc5n4C[C@@H]4CCO4)CC3)n1)CC2] | (S)-2-((4-(6-(6-cyano-1,2,4,5-tetrahydro- 3H-benzo[d]azepin-3-yl)pyridin-2- yl)piperazin-1-yl)methyl)-1-(oxetan-2- ylmethyl)-1H-benzo[d]imidazole-6- carboxylic acid |
| 80 | 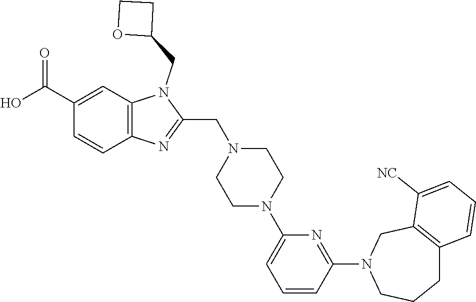 [SMILES: N#Cc1cccc2c1CN(c1cccc(N3CCN(Cc4nc5ccc(C(=O)O)cc5n4C[C@@H]4CCO4)CC3)n1)CCC2] | (S)-2-((4-(6-(9-cyano-1,3,4,5-tetrahydro- 2H-benzo[c]azepin-2-yl)pyridin-2- yl)piperazin-1-yl)methyl)-1-(oxetan-2- ylmethyl)-1H-benzo[d]imidazole-6- carboxylic acid |
| 81 | 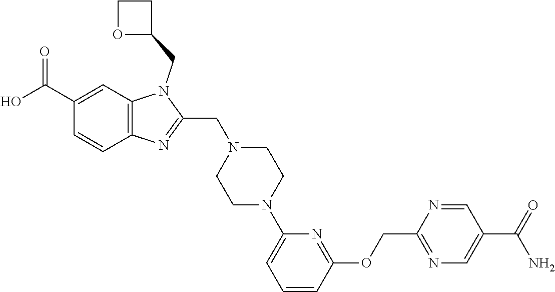 [SMILES: NC(=O)c1cnc(COc2cccc(N3CCN(Cc4nc5ccc(C(=O)O)cc5n4C[C@@H]4CCO4)CC3)n2)nc1] | (S)-2-((4-(6-((5-carbamoylpyrimidin-2- yl)methoxy)pyridin-2-yl)piperazin-1- yl)methyl)-1-(oxetan-2-ylmethyl)-1H- benzo[d]imidazole-6-carboxylic acid |
| 82 | 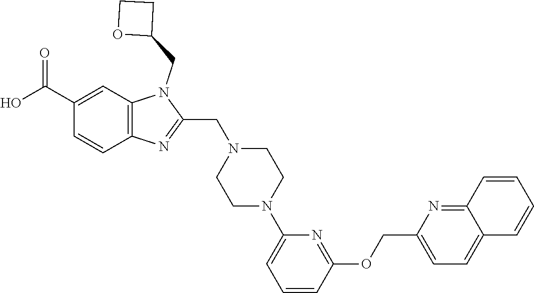 [SMILES: O=C(O)c1ccc2nc(CN3CCN(c4cccc(OCc5ccc6ccccc6n5)n4)CC3)n(C[C@@H]3CCO3)c2c1] | (S)-1-(oxetan-2-ylmethyl)-2-((4-(6- (quinolin-2-ylmethoxy)pyridin-2- yl)piperazin-1-yl)methyl)-1H- benzo[d]imidazole-6-carboxylic acid |
| 83 | 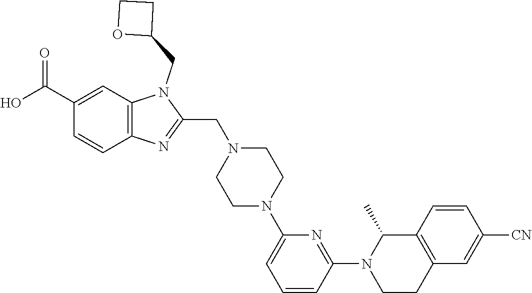 [SMILES: C[C@@H]1c2ccc(C#N)cc2CCN1c1cccc(N2CCN(Cc3nc4ccc(C(=O)O)cc4n3C[C@@H]3CCO3)CC2)n1] | 2-((4-(6-((R)-6-cyano-1-methyl-3,4- dihydroisoquinolin-2(1H)-yl)pyridin-2- yl)piperazin-1-yl)methyl)-1-(((S)-oxetan- 2-yl)methyl)-1H-benzo[d]imidazole-6- carboxylic acid |
| 84 | 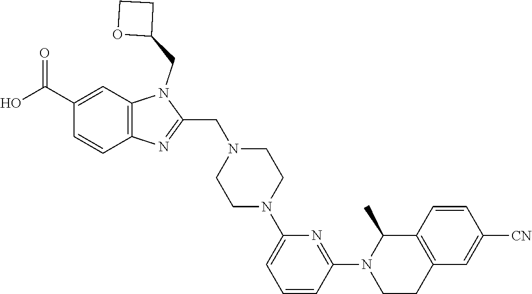 [SMILES: C[C@H]1c2ccc(C#N)cc2CCN1c1cccc(N2CCN(Cc3nc4ccc(C(=O)O)cc4n3C[C@@H]3CCO3)CC2)n1] | 2-((4-(6-((S)-6-cyano-1-methyl-3,4- dihydroisoquinolin-2(1H)-yl)pyridin-2- yl)piperazin-1-yl)methyl)-1-(((S)-oxetan- 2-yl)methyl)-1H-benzo[d]imidazole-6- carboxylic acid |
| 85 | 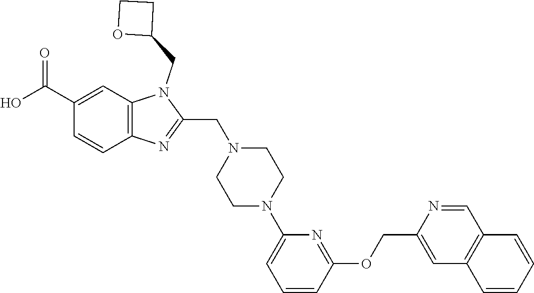 [SMILES: O=C(O)c1ccc2nc(CN3CCN(c4cccc(OCc5cc6ccccc6cn5)n4)CC3)n(C[C@@H]3CCO3)c2c1] | (S)-2-((4-(6-(isoquinolin-3- ylmethoxy)pyridin-2-yl)piperazin-1- yl)methyl)-1-(oxetan-2-ylmethyl)-1H- benzo[d]imidazole-6-carboxylic acid |
| 86 | 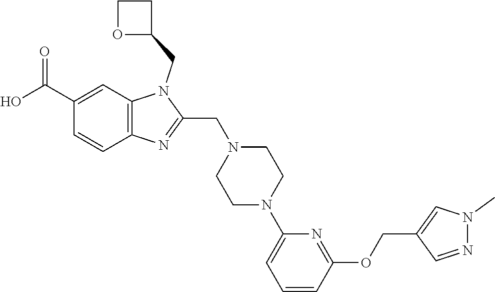 [SMILES: Cn1cc(COc2cccc(N3CCN(Cc4nc5ccc(C(=O)O)cc5n4C[C@@H]4CCO4)CC3)n2)cn1] | (S)-2-((4-(6-((1-methyl-1H-pyrazol-4- yl)methoxy)pyridin-2-yl)piperazin-1- yl)methyl)-1-(oxetan-2-ylmethyl)-1H- benzo[d]imidazole-6-carboxylic acid |
| 87 | 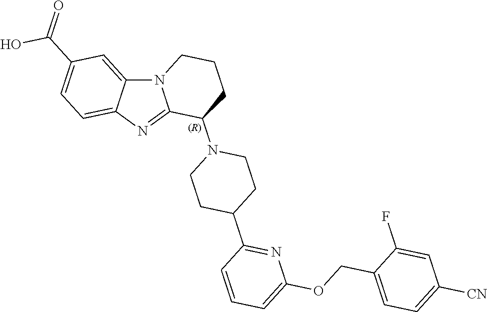 | (R)-4-(4-(6-((4-cyano-2- fluorobenzyl)oxy)pyridin-2-yl)piperidin-1- yl)-1,2,3,4- tetrahydrobenzo[4,5]imidazo[1,2- a]pyridine-8-carboxylic acid |
| 88 | 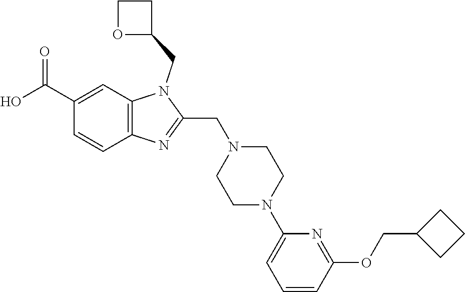 [SMILES: O=C(O)c1ccc2nc(CN3CCN(c4cccc(OCC5CCC5)n4)CC3)n(C[C@@H]3CCO3)c2c1] | (S)-2-((4-(6-(cyclobutylmethoxy)pyridin- 2-yl)piperazin-1-yl)methyl)-1-(oxetan-2- ylmethyl)-1H-benzo[d]imidazole-6- carboxylic acid |
| 89 | 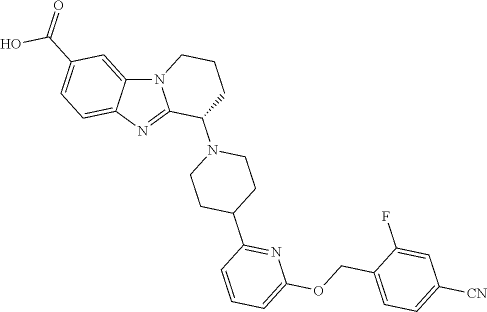 [SMILES: N#Cc1ccc(COc2cccc(C3CCN([C@H]4CCCn5c4nc4ccc(C(=O)O)cc45)CC3)n2)c(F)c1] | (S)-4-(4-(6-((4-cyano-2- fluorobenzyl)oxy)pyridin-2-yl)piperidin-1- yl)-1,2,3,4- tetrahydrobenzo[4,5]imidazo[1,2- a]pyridine-8-carboxylic acid |
| 90 | 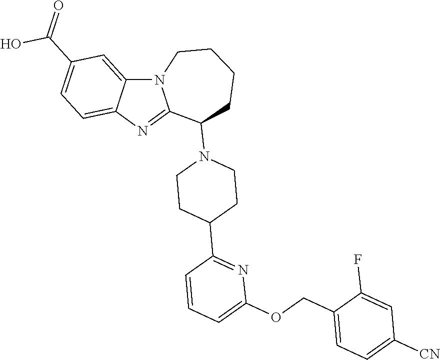 [SMILES: N#Cc1ccc(COc2cccc(C3CCN([C@@H]4CCCCn5c4nc4ccc(C(=O)O)cc45)CC3)n2)c(F)c1] | (R)-6-(4-(6-((4-cyano-2- fluorobenzyl)oxy)pyridin-2-yl)piperidin-1- yl)-7,8,9,10-tetrahydro-6H- benzo[4,5]imidazo[1,2-a]azepine-2- carboxylic acid |
| 91 | 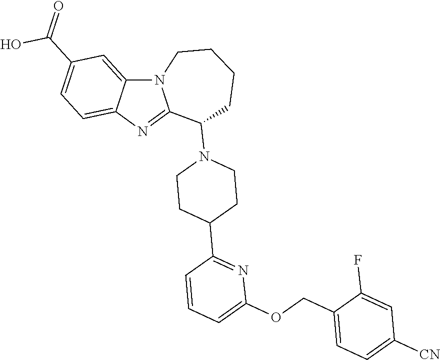 [SMILES: N#Cc1ccc(COc2cccc(C3CCN([C@H]4CCCCn5c4nc4ccc(C(=O)O)cc45)CC3)n2)c(F)c1] | (S)-6-(4-(6-((4-cyano-2- fluorobenzyl)oxy)pyridin-2-yl)piperidin-1- yl)-7,8,9,10-tetrahydro-6H- benzo[4,5]imidazo[1,2-a]azepine-2- carboxylic acid |
| 92 | 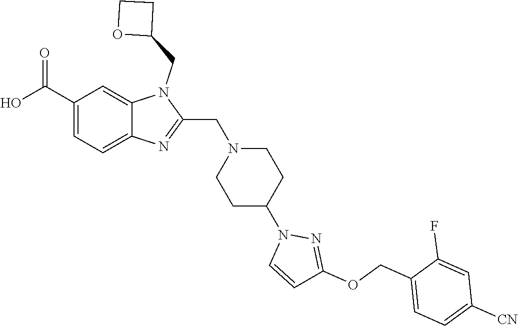 [SMILES: N#Cc1ccc(COc2ccn(C3CCN(Cc4nc5ccc(C(=O)O)cc5n4C[C@@H]4CCO4)CC3)n2)c(F)c1] | (S)-2-((4-(3-((4-cyano-2- fluorobenzyl)oxy)-1H-pyrazol-1- yl)piperidin-1-yl)methyl)-1-(oxetan-2- ylmethyl)-1H-benzo[d]imidazole-6- carboxylic acid |
| 93 | 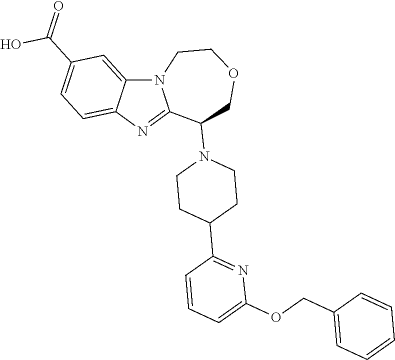 [SMILES: O=C(O)c1ccc2nc3n(c2c1)CCOC[C@H]3N1CCC(c2cccc(OCc3ccccc3)n2)CC1] | (S)-5-(4-(6-(benzyloxy)pyridin-2- yl)piperidin-1-yl)-1,2,4,5- tetrahydrobenzo[4,5]imidazo[1,2- d][1,4]oxazepine-9-carboxylic acid |
| 94 | 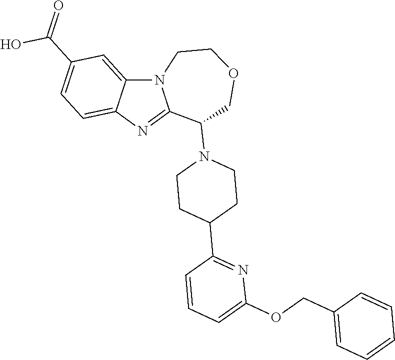 [SMILES: O=C(O)c1ccc2nc3n(c2c1)CCOC[C@@H]3N1CCC(c2cccc(OCc3ccccc3)n2)CC1] | (R)-5-(4-(6-(benzyloxy)pyridin-2- yl)piperidin-1-yl)-1,2,4,5- tetrahydrobenzo[4,5]imidazo[1,2- d][1,4]oxazepine-9-carboxylic acid |
| 95 | 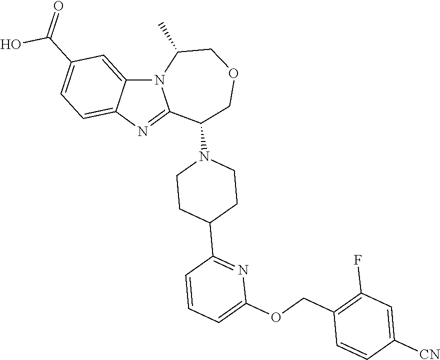 [SMILES: C[C@@H]1COC[C@H](N2CCC(c3cccc(OCc4ccc(C#N)cc4F)n3)CC2)c2nc3ccc(C(=O)O)cc3n21] | (1R,5R)-5-(4-(6-((4-cyano-2- fluorobenzyl)oxy)pyridin-2-yl)piperidin-1- yl)-1-methyl-1,2,4,5- tetrahydrobenzo[4,5]imidazo[1,2- d][1,4]oxazepine-9-carboxylic acid |
| 96 | 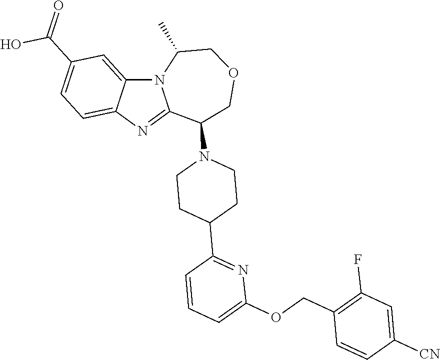 [SMILES: C[C@@H]1COC[C@@H](N2CCC(c3cccc(OCc4ccc(C#N)cc4F)n3)CC2)c2nc3ccc(C(=O)O)cc3n21] | (1R,5S)-5-(4-(6-((4-cyano-2- fluorobenzyl)oxy)pyridin-2-yl)piperidin-1- yl)-1-methyl-1,2,4,5- tetrahydrobenzo[4,5]imidazo[1,2- d][1,4]oxazepine-9-carboxylic acid |
| 97 | 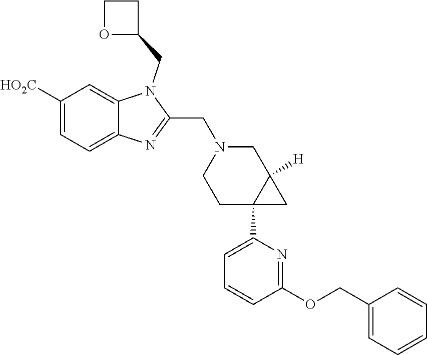 [SMILES: O=C(O)c1ccc2nc(CN3CC[C@]4(c5cccc(OCc6ccccc6)n5)C[C@@H]4C3)n(C[C@@H]3CCO3)c2c1] | 2-(((1S,6R)-6-(6-(benzyloxy)pyridin-2- yl)-3-azabicyclo[4.1.0]heptan-3- yl)methyl)-1-(((S)-oxetan-2-yl)methyl)- 1H-benzo[d]imidazole-6-carboxylic acid |
| 98 | 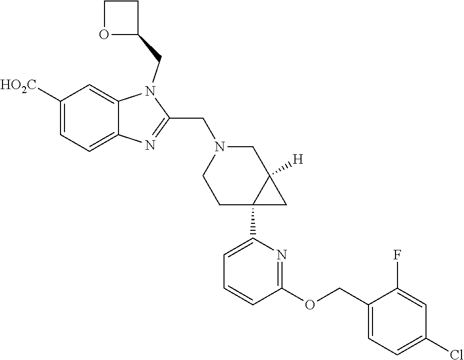 [SMILES: O=C(O)c1ccc2nc(CN3CC[C@]4(c5cccc(OCc6ccc(Cl)cc6F)n5)C[C@@H]4C3)n(C[C@@H]3CCO3)c2c1] | 2-(((1S,6R)-6-(6-((4-chloro-2- fluorobenzyl)oxy)pyridin-2-yl)-3- azabicyclo[4.1.0]heptan-3-yl)methyl)-1- (((S)-oxetan-2-yl)methyl)-1H- benzo[d]imidazole-6-carboxylic acid |
| 99 | 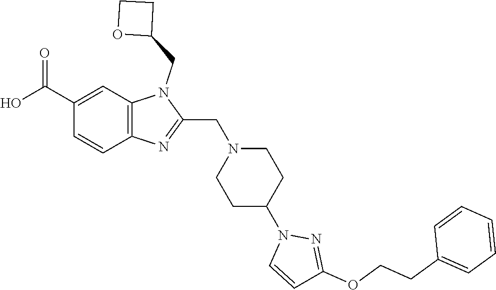 [SMILES: O=C(O)c1ccc2nc(CN3CCC(n4ccc(OCCc5ccccc5)n4)CC3)n(C[C@@H]3CCO3)c2c1] | (S)-1-(oxetan-2-ylmethyl)-2-((4-(3- phenethoxy-1H-pyrazol-1-yl)piperidin-1- yl)methyl)-1H-benzo[d]imidazole-6- carboxylic acid |
| 100 | 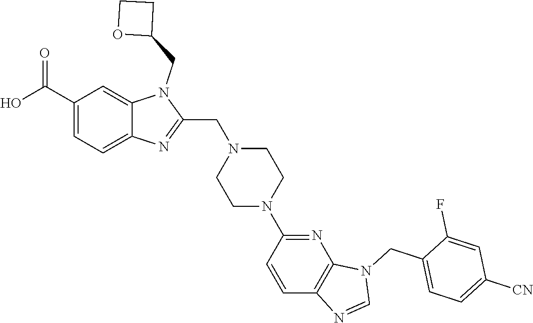 [SMILES: N#Cc1ccc(Cn2cnc3ccc(N4CCN(Cc5nc6ccc(C(=O)O)cc6n5C[C@@H]5CCO5)CC4)nc32)c(F)c1] | (S)-2-((4-(3-(4-cyano-2-fluorobenzyl)-3H- imidazo[4,5-b]pyridin-5-yl)piperazin-1- yl)methyl)-1-(oxetan-2-ylmethyl)-1H- benzo[d]imidazole-6-carboxylic acid |
| 101 | 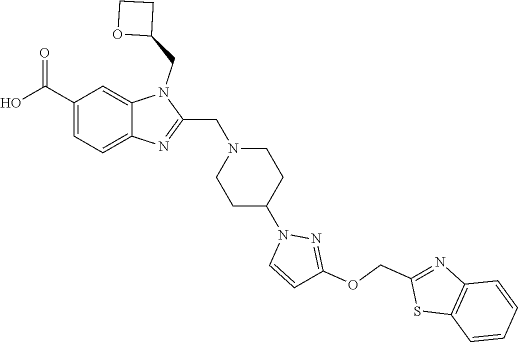 [SMILES: O=C(O)c1ccc2nc(CN3CCC(n4ccc(OCc5nc6ccccc6s5)n4)CC3)n(C[C@@H]3CCO3)c2c1] | (S)-2-((4-(3-(benzo[d]thiazol-2- ylmethoxy)-1H-pyrazol-1-yl)piperidin-1- yl)methyl)-1-(oxetan-2-ylmethyl)-1H- benzo[d]imidazole-6-carboxylic acid |
| 102 | 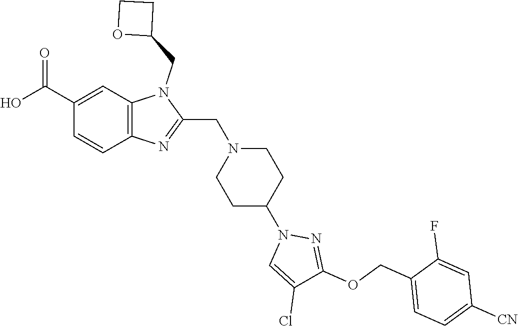 [SMILES: N#Cc1ccc(COc2nn(C3CCN(Cc4nc5ccc(C(=O)O)cc5n4C[C@@H]4CCO4)CC3)cc2Cl)c(F)c1] | (S)-2-((4-(4-chloro-3-((4-cyano-2- fluorobenzyl)oxy)-1H-pyrazol-1- yl)piperidin-1-yl)methyl)-1-(oxetan-2- ylmethyl)-1H-benzo[d]imidazole-6- carboxylic acid |
| 103 | 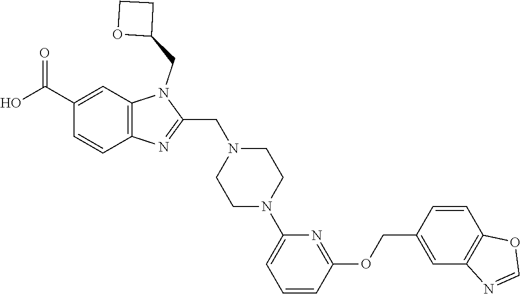 [SMILES: O=C(O)c1ccc2nc(CN3CCN(c4cccc(OCc5ccc6ocnc6c5)n4)CC3)n(C[C@@H]3CCO3)c2c1] | (S)-2-((4-(6-(benzo[d]oxazol-5- ylmethoxy)pyridin-2-yl)piperazin-1- yl)methyl)-1-(oxetan-2-ylmethyl)-1H- benzo[d]imidazole-6-carboxylic acid |
| 104-P1 | 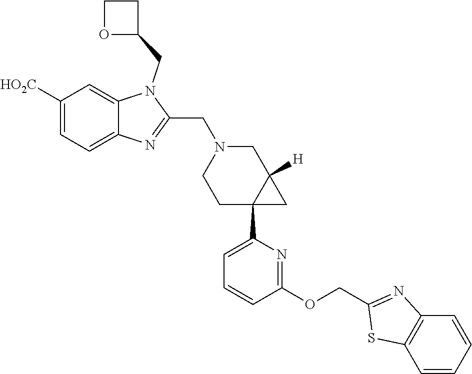 [SMILES: O=C(O)c1ccc2nc(CN3CC[C@@]4(c5cccc(OCc6nc7ccccc7s6)n5)C[C@H]4C3)n(C[C@@H]3CCO3)c2c1] | 2-(((1R,6S)-6-(6-(benzo[d]thiazol-2- ylmethoxy)pyridin-2-yl)-3- azabicyclo[4.1.0]heptan-3-yl)methyl)-1- (((S)-oxetan-2-yl)methyl)-1H- benzo[d]imidazole-6-carboxylic acid |
| 104-P2 | 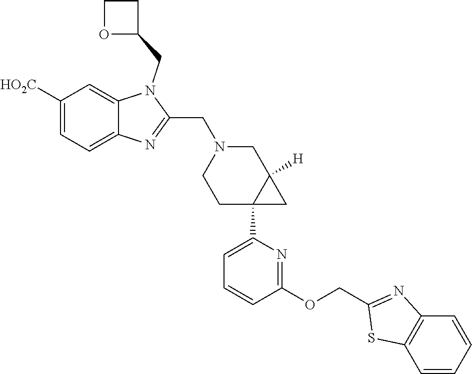 [SMILES: O=C(O)c1ccc2nc(CN3CC[C@]4(c5cccc(OCc6nc7ccccc7s6)n5)C[C@@H]4C3)n(C[C@@H]3CCO3)c2c1] | 2-(((1S,6R)-6-(6-(benzo[d]thiazol-2- ylmethoxy)pyridin-2-yl)-3- azabicyclo[4.1.0]heptan-3-yl)methyl)-1- (((S)-oxetan-2-yl)methyl)-1H- benzo[d]imidazole-6-carboxylic acid |
| 105 | 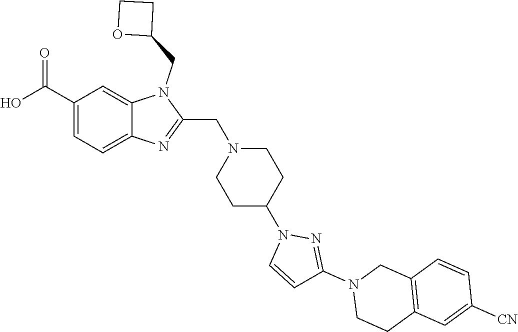 [SMILES: N#Cc1ccc2c(c1)CCN(c1ccn(C3CCN(Cc4nc5ccc(C(=O)O)cc5n4C[C@@H]4CCO4)CC3)n1)C2] | (S)-2-((4-(3-(6-cyano-3,4- dihydroisoquinolin-2(1H)-yl)-1H-pyrazol- 1-yl)piperidin-1-yl)methyl)-1-(oxetan-2- ylmethyl)-1H-benzo[d]imidazole-6- carboxylic acid |
| 106 | 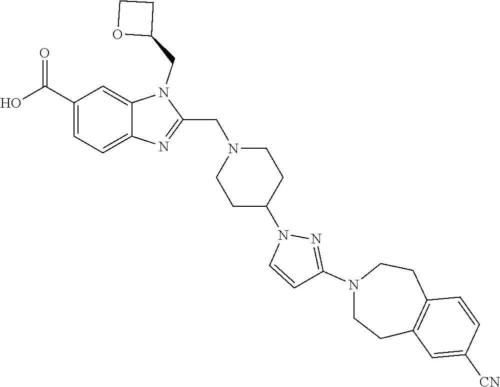 [SMILES: N#Cc1ccc2c(c1)CCN(c1ccn(C3CCN(Cc4nc5ccc(C(=O)O)cc5n4C[C@@H]4CCO4)CC3)n1)CC2] | (S)-2-((4-(3-(7-cyano-1,2,4,5-tetrahydro- 3H-benzo[d]azepin-3-yl)-1H-pyrazol-1- yl)piperidin-1-yl)methyl)-1-(oxetan-2- ylmethyl)-1H-benzo[d]imidazole-6- carboxylic acid |
| 107 | 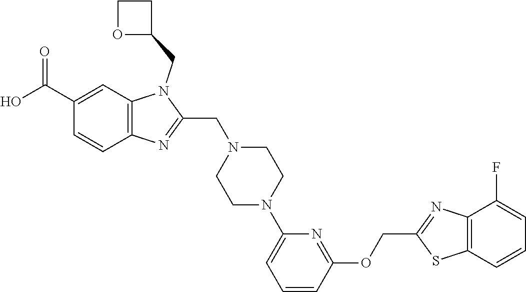 [SMILES: O=C(O)c1ccc2nc(CN3CCN(c4cccc(OCc5nc6c(F)cccc6s5)n4)CC3)n(C[C@@H]3CCO3)c2c1] | (S)-2-((4-(6-((4-fluorobenzo[d]thiazol-2- yl)methoxy)pyridin-2-yl)piperazin-1- yl)methyl)-1-(oxetan-2-ylmethyl)-1H- benzo[d]imidazole-6-carboxylic acid |
| 108 | 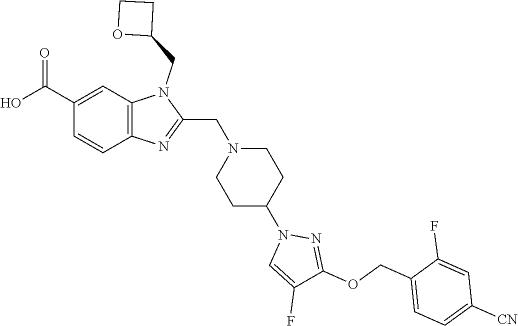 [SMILES: N#Cc1ccc(COc2nn(C3CCN(Cc4nc5ccc(C(=O)O)cc5n4C[C@@H]4CCO4)CC3)cc2F)c(F)c1] | (S)-2-((4-(3-((4-cyano-2- fluorobenzyl)oxy)-4-fluoro-1H-pyrazol-1- yl)piperidin-1-yl)methyl)-1-(oxetan-2- ylmethyl)-1H-benzo[d]imidazole-6- carboxylic acid |
| 109 | 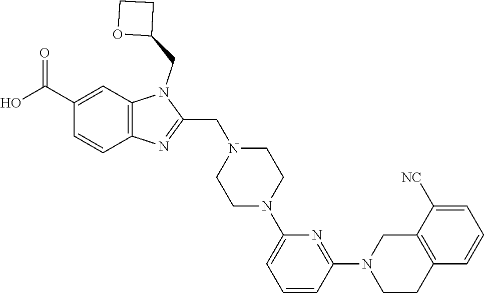 [SMILES: N#Cc1cccc2c1CN(c1cccc(N3CCN(Cc4nc5ccc(C(=O)O)cc5n4C[C@@H]4CCO4)CC3)n1)CC2] | (S)-2-((4-(6-(8-cyano-3,4- dihydroisoquinolin-2(1H)-yl)pyridin-2- yl)piperazin-1-yl)methyl)-1-(oxetan-2- ylmethyl)-1H-benzo[d]imidazole-6- carboxylic acid |
| 110 | 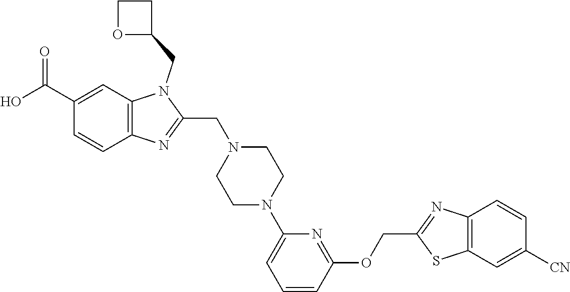 [SMILES: N#Cc1ccc2nc(COc3cccc(N4CCN(Cc5nc6ccc(C(=O)O)cc6n5C[C@@H]5CCO5)CC4)n3)sc2c1] | (S)-2-((4-(6-((6-cyanobenzo[d]thiazol-2- yl)methoxy)pyridin-2-yl)piperazin-1- yl)methyl)-1-(oxetan-2-ylmethyl)-1H- benzo[d]imidazole-6-carboxylic acid |
| 111 | 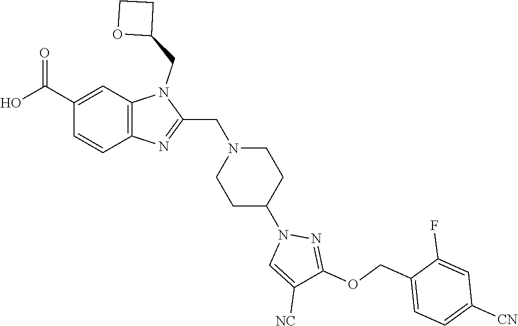 [SMILES: N#Cc1ccc(COc2nn(C3CCN(Cc4nc5ccc(C(=O)O)cc5n4C[C@@H]4CCO4)CC3)cc2C#N)c(F)c1] | (S)-2-((4-(4-cyano-3-((4-cyano-2- fluorobenzyl)oxy)-1H-pyrazol-1- yl)piperidin-1-yl)methyl)-1-(oxetan-2- ylmethyl)-1H-benzo[d]imidazole-6- carboxylic acid |
| 112 | 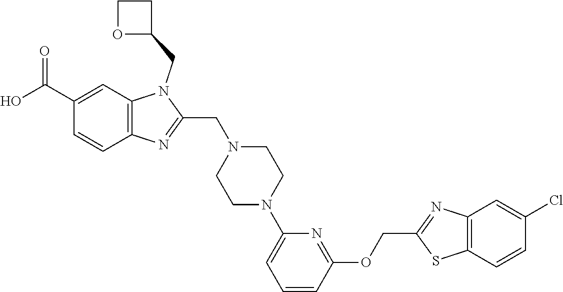 [SMILES: O=C(O)c1ccc2nc(CN3CCN(c4cccc(OCc5nc6cc(Cl)ccc6s5)n4)CC3)n(C[C@@H]3CCO3)c2c1] | (S)-2-((4-(6-((5-chlorobenzo[d]thiazol-2- yl)methoxy)pyridin-2-yl)piperazin-1- yl)methyl)-1-(oxetan-2-ylmethyl)-1H- benzo[d]imidazole-6-carboxylic acid |
| 113 | 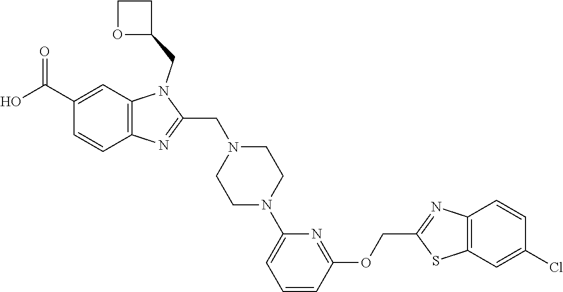 [SMILES: O=C(O)c1ccc2nc(CN3CCN(c4cccc(OCc5nc6ccc(Cl)cc6s5)n4)CC3)n(C[C@@H]3CCO3)c2c1] | (S)-2-((4-(6-((6-chlorobenzo[d]thiazol-2- yl)methoxy)pyridin-2-yl)piperazin-1- yl)methyl)-1-(oxetan-2-ylmethyl)-1H- benzo[d]imidazole-6-carboxylic acid |
| 114 | 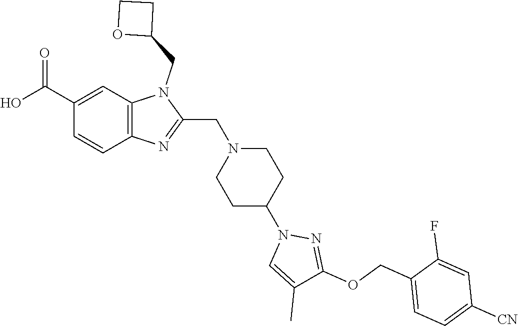 [SMILES: Cc1cn(C2CCN(Cc3nc4ccc(C(=O)O)cc4n3C[C@@H]3CCO3)CC2)nc1OCc1ccc(C#N)cc1F] | (S)-2-((4-(3-((4-cyano-2- fluorobenzyl)oxy)-4-methyl-1H-pyrazol- 1-yl)piperidin-1-yl)methyl)-1-(oxetan-2- ylmethyl)-1H-benzo[d]imidazole-6- carboxylic acid |
| 115 | 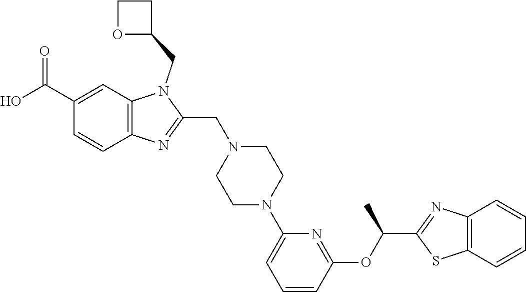 [SMILES: C[C@H](Oc1cccc(N2CCN(Cc3nc4ccc(C(=O)O)cc4n3C[C@@H]3CCO3)CC2)n1)c1nc2ccccc2s1] | 2-((4-(6-((S)-1-(benzo[d]thiazol-2- yl)ethoxy)pyridin-2-yl)piperazin-1- yl)methyl)-1-(((S)-oxetan-2-yl)methyl)- 1H-benzo[d]imidazole-6-carboxylic acid |
| 116 | 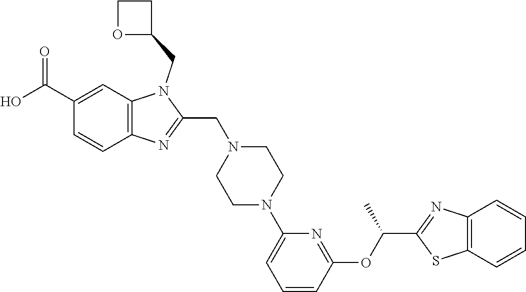 [SMILES: C[C@@H](Oc1cccc(N2CCN(Cc3nc4ccc(C(=O)O)cc4n3C[C@@H]3CCO3)CC2)n1)c1nc2ccccc2s1] | 2-((4-(6-((R)-1-(benzo[d]thiazol-2- yl)ethoxy)pyridin-2-yl)piperazin-1- yl)methyl)-1-(((S)-oxetan-2-yl)methyl)- 1H-benzo[d]imidazole-6-carboxylic acid |
| 117 | 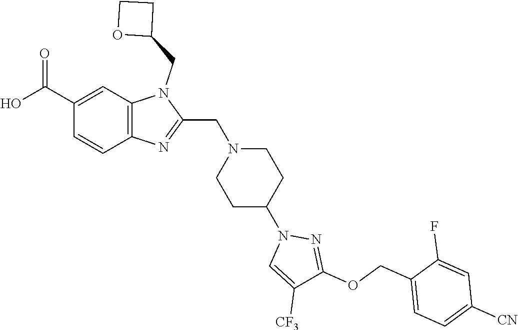 [SMILES: N#Cc1ccc(COc2nn(C3CCN(Cc4nc5ccc(C(=O)O)cc5n4C[C@@H]4CCO4)CC3)cc2C(F)(F)F)c(F)c1] | (S)-2-((4-(3-((4-cyano-2- fluorobenzyl)oxy)-4-(trifluoromethyl)-1H- pyrazol-1-yl)piperidin-1-yl)methyl)-1- (oxetan-2-ylmethyl)-1H- benzo[d]imidazole-6-carboxylic acid |
| 118 | 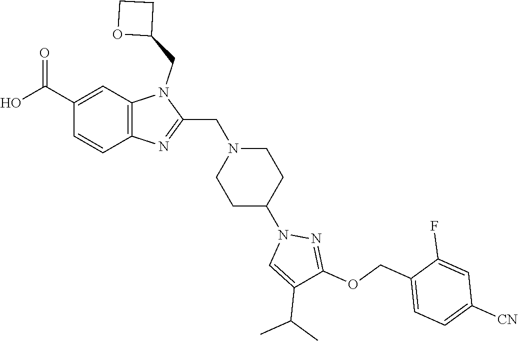 [SMILES: CC(C)c1cn(C2CCN(Cc3nc4ccc(C(=O)O)cc4n3C[C@@H]3CCO3)CC2)nc1OCc1ccc(C#N)cc1F] | (S)-2-((4-(3-((4-cyano-2- fluorobenzyl)oxy)-4-isopropyl-1H- pyrazol-1-yl)piperidin-1-yl)methyl)-1- (oxetan-2-ylmethyl)-1H- benzo[d]imidazole-6-carboxylic acid |
| 119 | 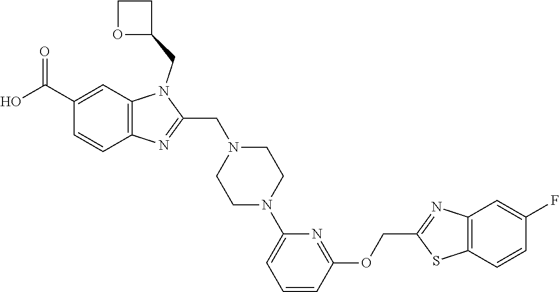 [SMILES: O=C(O)c1ccc2nc(CN3CCN(c4cccc(OCc5nc6cc(F)ccc6s5)n4)CC3)n(C[C@@H]3CCO3)c2c1] | (S)-2-((4-(6-((5-fluorobenzo[d]thiazol-2- yl)methoxy)pyridin-2-yl)piperazin-1- yl)methyl)-1-(oxetan-2-ylmethyl)-1H- benzo[d]imidazole-6-carboxylic acid |
| 120 | 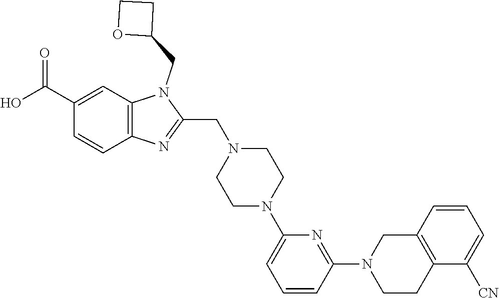 [SMILES: N#Cc1cccc2c1CCN(c1cccc(N3CCN(Cc4nc5ccc(C(=O)O)cc5n4C[C@@H]4CCO4)CC3)n1)C2] | (S)-2-((4-(6-(5-cyano-3,4- dihydroisoquinolin-2(1H)-yl)pyridin-2- yl)piperazin-1-yl)methyl)-1-(oxetan-2- ylmethyl)-1H-benzo[d]imidazole-6- carboxylic acid |
| 121-P1 | 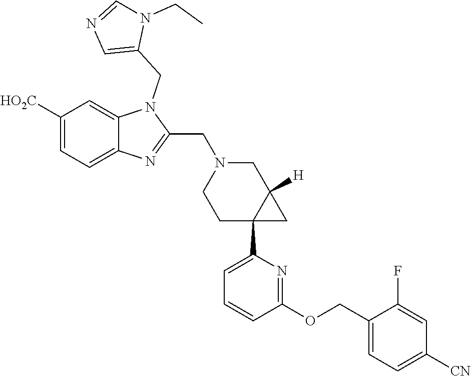 [SMILES: CCn1cncc1Cn1c(CN2CC[C@@]3(c4cccc(OCc5ccc(C#N)cc5F)n4)C[C@H]3C2)nc2ccc(C(=O)O)cc21] | 2-(((1R,6S)-6-(6-((4-cyano-2- fluorobenzyl)oxy)pyridin-2-yl)-3- azabicyclo[4.1.0]heptan-3-yl)methyl)-1- ((1-ethyl-1H-imidazol-5-yl)methyl)-1H- benzo[d]imidazole-6-carboxylic acid |
| 121-P2 | 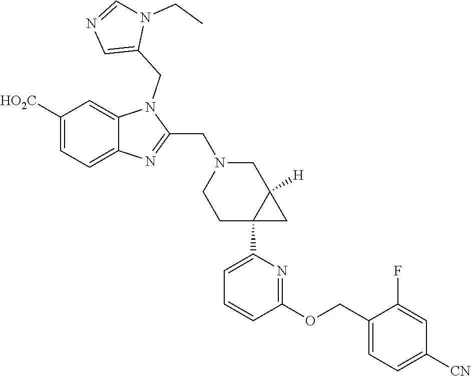 [SMILES: CCn1cncc1Cn1c(CN2CC[C@]3(c4cccc(OCc5ccc(C#N)cc5F)n4)C[C@@H]3C2)nc2ccc(C(=O)O)cc21] | 2-(((1S,6R)-6-(6-((4-cyano-2- fluorobenzyl)oxy)pyridin-2-yl)-3- azabicyclo[4.1.0]heptan-3-yl)methyl)-1- ((1-ethyl-1H-imidazol-5-yl)methyl)-1H- benzo[d]imidazole-6-carboxylic acid |
| 122 | 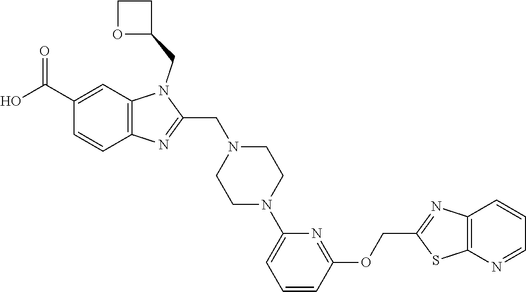 [SMILES: O=C(O)c1ccc2nc(CN3CCN(c4cccc(OCc5nc6cccnc6s5)n4)CC3)n(C[C@@H]3CCO3)c2c1] | (S)-1-(oxetan-2-ylmethyl)-2-((4-(6- (thiazolo[5,4-b]pyridin-2- ylmethoxy)pyridin-2-yl)piperazin-1- yl)methyl)-1H-benzo[d]imidazole-6- carboxylic acid |
| 123 | 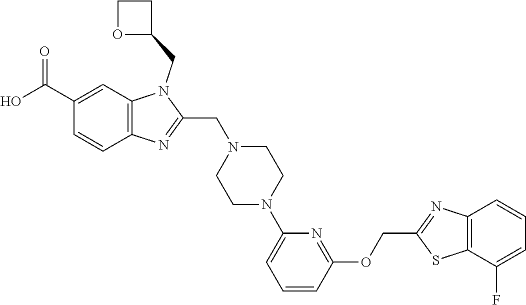 [SMILES: O=C(O)c1ccc2nc(CN3CCN(c4cccc(OCc5nc6cccc(F)c6s5)n4)CC3)n(C[C@@H]3CCO3)c2c1] | (S)-2-((4-(6-((7-fluorobenzo[d]thiazol-2- yl)methoxy)pyridin-2-yl)piperazin-1- yl)methyl)-1-(oxetan-2-ylmethyl)-1H- benzo[d]imidazole-6-carboxylic acid |
| 124 | 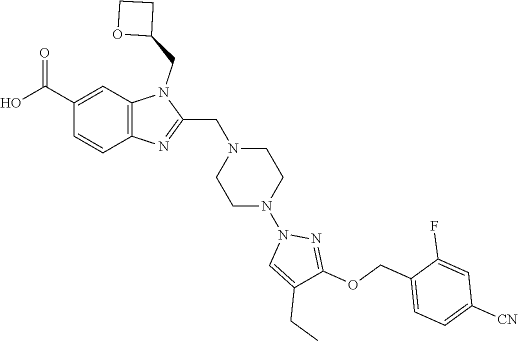 [SMILES: CCc1cn(N2CCN(Cc3nc4ccc(C(=O)O)cc4n3C[C@@H]3CCO3)CC2)nc1OCc1ccc(C#N)cc1F] | (S)-2-((4-(3-((4-cyano-2- fluorobenzyl)oxy)-4-ethyl-1H-pyrazol-1- yl)piperidin-1-yl)methyl)-1-(oxetan-2- ylmethyl)-1H-benzo[d]imidazole-6- carboxylic acid |
| 125 | 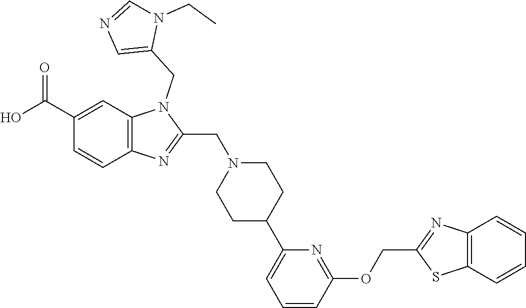 [SMILES: CCn1cncc1Cn1c(CN2CCC(c3cccc(OCc4nc5ccccc5s4)n3)CC2)nc2ccc(C(=O)O)cc21] | 2-((4-(6-(benzo[d]thiazol-2- ylmethoxy)pyridin-2-yl)piperidin-1- yl)methyl)-1-((1-ethyl-1H-imidazol-5- yl)methyl)-1H-benzo[d]imidazole-6- carboxylic acid |
| 126-P1 | 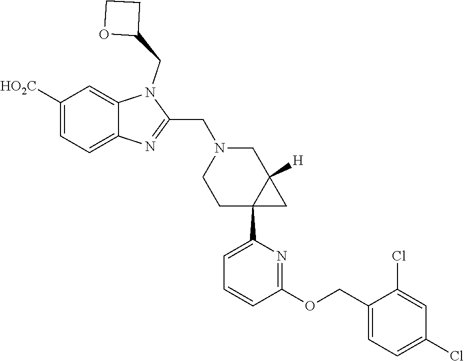 | 2-(((1R,6S)-6-(6-((2,4- dichlorobenzyl)oxy)pyridin-2-yl)-3- azabicyclo[4.1.0]heptan-3-yl)methyl)-1- (((R)-oxetan-2-yl)methyl)-1H- benzo[d]imidazole-6-carboxylic acid |
| 126-P2 | 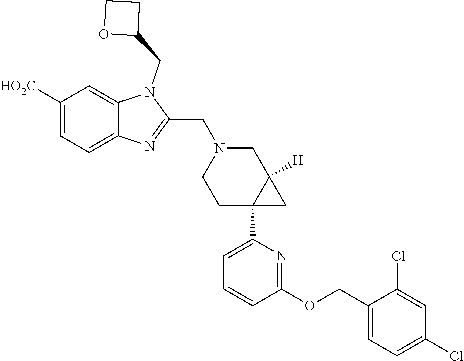 | 2-(((1S,6R)-6-(6-((2,4- dichlorobenzyl)oxy)pyridin-2-yl)-3- azabicyclo[4.1.0]heptan-3-yl)methyl)-1- (((R)-oxetan-2-yl)methyl)-1H- benzo[d]imidazole-6-carboxylic acid |
| 127 | 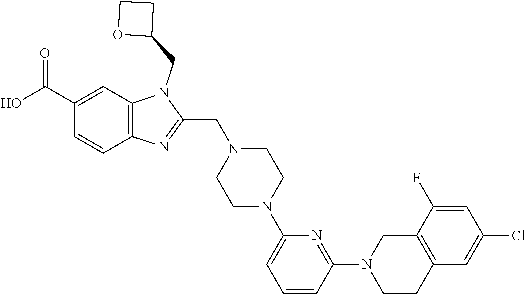 [SMILES: O=C(O)c1ccc2nc(CN3CCN(c4cccc(N5CCc6cc(Cl)cc(F)c6C5)n4)CC3)n(C[C@@H]3CCO3)c2c1] | (S)-2-((4-(6-(6-chloro-8-fluoro-3,4- dihydroisoquinolin-2(1H)-yl)pyridin-2- yl)piperazin-1-yl)methyl)-1-(oxetan-2- ylmethyl)-1H-benzo[d]imidazole-6- carboxylic acid |
| 128 | 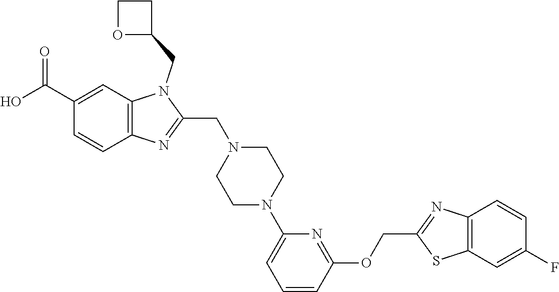 [SMILES: O=C(O)c1ccc2nc(CN3CCN(c4cccc(OCc5nc6ccc(F)cc6s5)n4)CC3)n(C[C@@H]3CCO3)c2c1] | (S)-2-((4-(6-((6-fluorobenzo[d]thiazol-2- yl)methoxy)pyridin-2-yl)piperazin-1- yl)methyl)-1-(oxetan-2-ylmethyl)-1H- benzo[d]imidazole-6-carboxylic acid |
| 129 | 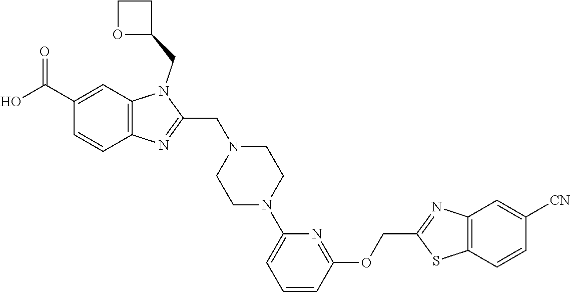 [SMILES: N#Cc1ccc2sc(COc3cccc(N4CCN(Cc5nc6ccc(C(=O)O)cc6n5C[C@@H]5CCO5)CC4)n3)nc2c1] | (S)-2-((4-(6-((5-cyanobenzo[d]thiazol-2- yl)methoxy)pyridin-2-yl)piperazin-1- yl)methyl)-1-(oxetan-2-ylmethyl)-1H- benzo[d]imidazole-6-carboxylic acid |
| 130 | 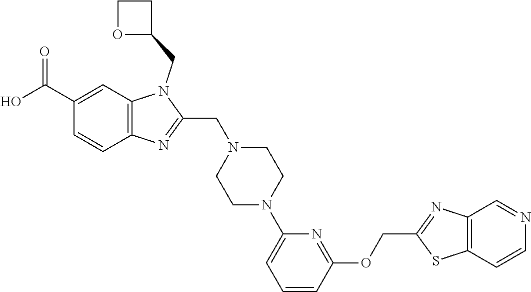 [SMILES: O=C(O)c1ccc2nc(CN3CCN(c4cccc(OCc5nc6cnccc6s5)n4)CC3)n(C[C@@H]3CCO3)c2c1] | (S)-1-(oxetan-2-ylmethyl)-2-((4-(6- (thiazolo[4,5-c]pyridin-2- ylmethoxy)pyridin-2-yl)piperazin-1- yl)methyl)-1H-benzo[d]imidazole-6- carboxylic acid |
| 131 | 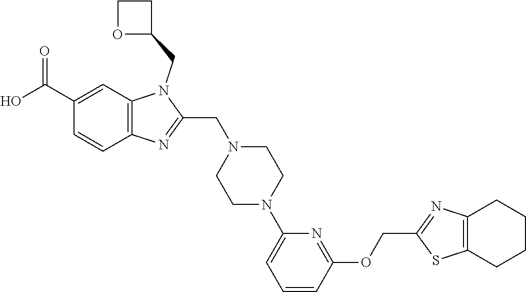 [SMILES: O=C(O)c1ccc2nc(CN3CCN(c4cccc(OCc5nc6c(s5)CCCC6)n4)CC3)n(C[C@@H]3CCO3)c2c1] | (S)-1-(oxetan-2-ylmethyl)-2-((4-(6- ((4,5,6,7-tetrahydrobenzo[d]thiazol-2- yl)methoxy)pyridin-2-yl)piperazin-1- yl)methyl)-1H-benzo[d]imidazole-6- carboxylic acid |
| 132 | 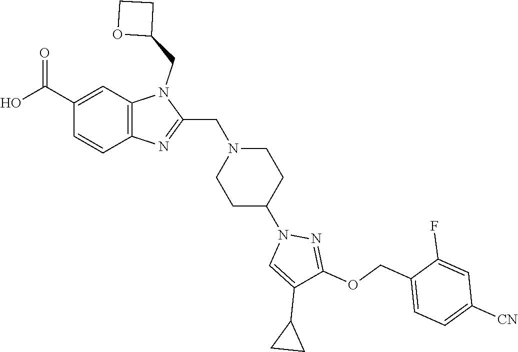 [SMILES: N#Cc1ccc(COc2nn(C3CCN(Cc4nc5ccc(C(=O)O)cc5n4C[C@@H]4CCO4)CC3)cc2C2CC2)c(F)c1] | (S)-2-((4-(3-((4-cyano-2- fluorobenzyl)oxy)-4-cyclopropyl-1H- pyrazol-1-yl)piperidin-1-yl)methyl)-1- (oxetan-2-ylmethyl)-1H- benzo[d]imidazole-6-carboxylic acid |
| 133 | 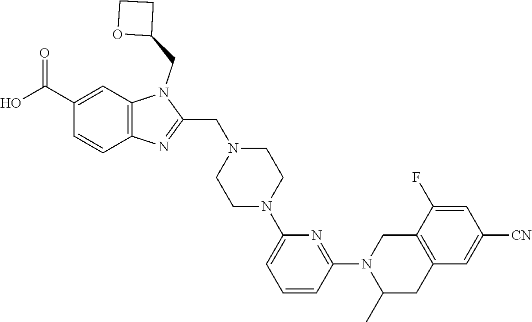 [SMILES: CC1Cc2cc(C#N)cc(F)c2CN1c1cccc(N2CCN(Cc3nc4ccc(C(=O)O)cc4n3C[C@@H]3CCO3)CC2)n1] | 2-((4-(6-(6-cyano-8-fluoro-3-methyl-3,4- dihydroisoquinolin-2(1H)-yl)pyridin-2- yl)piperazin-1-yl)methyl)-1-(((S)-oxetan- 2-yl)methyl)-1H-benzo[d]imidazole-6- carboxylic acid |
| 134 | 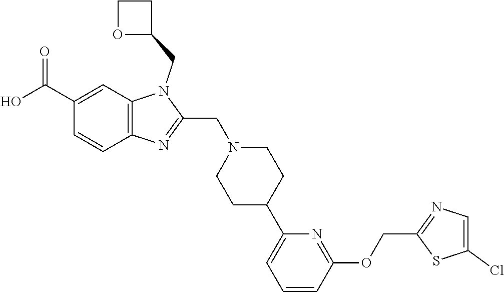 [SMILES: O=C(O)c1ccc2nc(CN3CCC(c4cccc(OCc5ncc(Cl)s5)n4)CC3)n(C[C@@H]3CCO3)c2c1] | (S)-2-((4-(6-((5-chlorothiazol-2- yl)methoxy)pyridin-2-yl)piperidin-1- yl)methyl)-1-(oxetan-2-ylmethyl)-1H- benzo[d]imidazole-6-carboxylic acid |
| 135 | 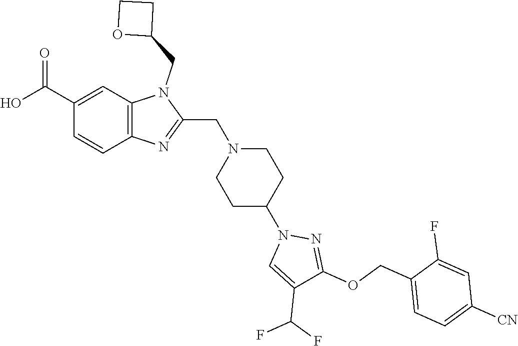 [SMILES: N#Cc1ccc(COc2nn(C3CCN(Cc4nc5ccc(C(=O)O)cc5n4C[C@@H]4CCO4)CC3)cc2C(F)F)c(F)c1] | (S)-2-((4-(3-((4-cyano-2- fluorobenzyl)oxy)-4-(difluoromethyl)-1H- pyrazol-1-yl)piperidin-1-yl)methyl)-1- (oxetan-2-ylmethyl)-1H- benzo[d]imidazole-6-carboxylic acid |
| 136 | 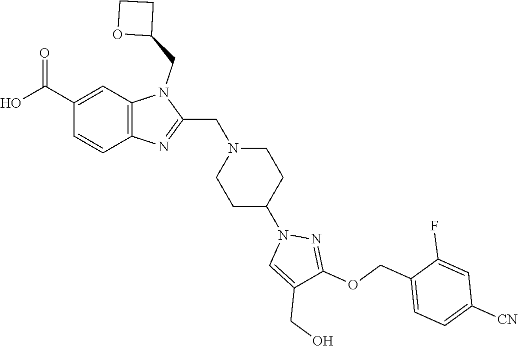 [SMILES: N#Cc1ccc(COc2nn(C3CCN(Cc4nc5ccc(C(=O)O)cc5n4C[C@@H]4CCO4)CC3)cc2CO)c(F)c1] | (S)-2-((4-(3-((4-cyano-2- fluorobenzyl)oxy)-4-(hydroxymethyl)-1H- pyrazol-1-yl)piperidin-1-yl)methyl)-1- (oxetan-2-ylmethyl)-1H- benzo[d]imidazole-6-carboxylic acid |
| 137 | 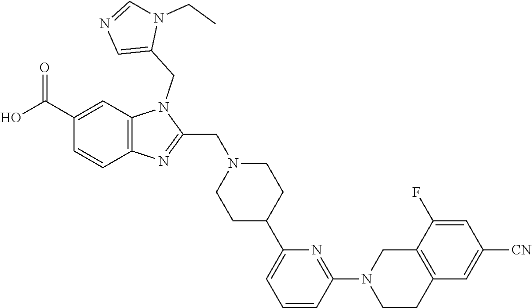 [SMILES: CCn1cncc1Cn1c(CN2CCC(c3cccc(N4CCc5cc(C#N)cc(F)c5C4)n3)CC2)nc2ccc(C(=O)O)cc21] | 2-((4-(6-(6-cyano-8-fluoro-3,4- dihydroisoquinolin-2(1H)-yl)pyridin-2- yl)piperidin-1-yl)methyl)-1-((1-ethyl-1H- imidazol-5-yl)methyl)-1H- benzo[d]imidazole-6-carboxylic acid |
| 138 | 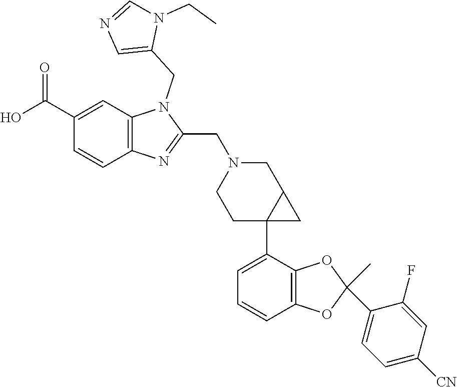 [SMILES: CCn1cncc1Cn1c(CN2CCC3(c4cccc5c4OC(C)(c4ccc(C#N)cc4F)O5)CC3C2)nc2ccc(C(=O)O)cc21] | 2-((6-(2-(4-cyano-2-fluorophenyl)-2- methylbenzo[d][1,3]dioxol-4-yl)-3- azabicyclo[4.1.0]heptan-3-yl)methyl)-1- ((1-ethyl-1H-imidazol-5-yl)methyl)-1H- benzo[d]imidazole-6-carboxylic acid |
| 139 | 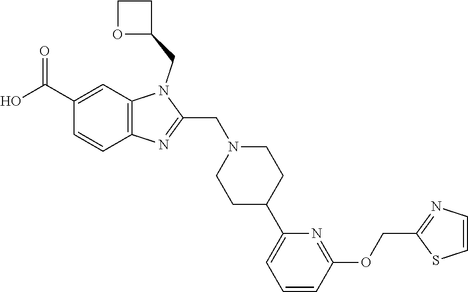 [SMILES: O=C(O)c1ccc2nc(CN3CCC(c4cccc(OCc5nccs5)n4)CC3)n(C[C@@H]3CCO3)c2c1] | (S)-1-(oxetan-2-ylmethyl)-2-((4-(6- (thiazol-2-ylmethoxy)pyridin-2- yl)piperidin-1-yl)methyl)-1H- benzo[d]imidazole-6-carboxylic acid |
| 140-P1 | 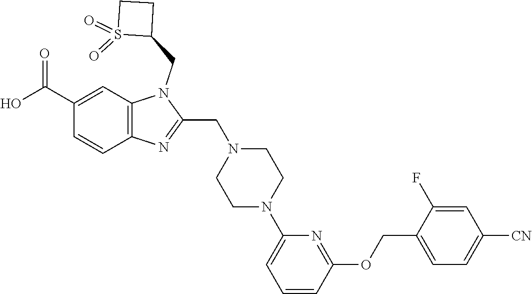 [SMILES: N#Cc1ccc(COc2cccc(N3CCN(Cc4nc5ccc(C(=O)O)cc5n4C[C@@H]4CCS4(=O)=O)CC3)n2)c(F)c1] | (S)-2-((4-(6-((4-cyano-2- fluorobenzyl)oxy)pyridin-2-yl)piperazin- 1-yl)methyl)-1-((1,1-dioxidothietan-2- yl)methyl)-1H-benzo[d]imidazole-6- carboxylic acid |
| 140-P2 | 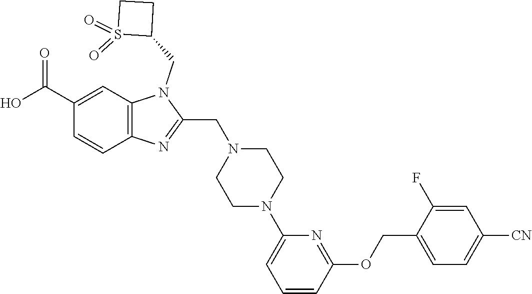 [SMILES: N#Cc1ccc(COc2cccc(N3CCN(Cc4nc5ccc(C(=O)O)cc5n4C[C@H]4CCS4(=O)=O)CC3)n2)c(F)c1] | (R)-2-((4-(6-((4-cyano-2- fluorobenzyl)oxy)pyridin-2-yl)piperazin- 1-yl)methyl)-1-((1,1-dioxidothietan-2- yl)methyl)-1H-benzo[d]imidazole-6- carboxylic acid |
| 141-P1 | 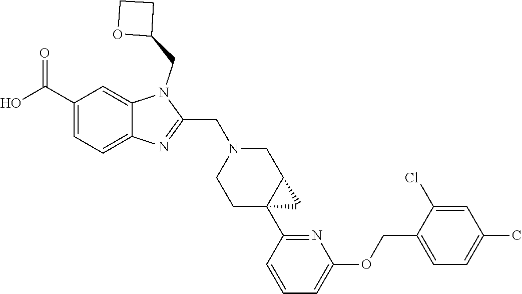 [SMILES: Cc1ccc(COc2cccc([C@@]34CCN(Cc5nc6ccc(C(=O)O)cc6n5C[C@@H]5CCO5)C[C@@H]3C4)n2)c(Cl)c1] | 2-(((1R,6S)-6-(6-((2,4- dichlorobenzyl)oxy)pyridin-2-yl)-3- azabicyclo[4.1.0]heptan-3-yl)methyl)-1- (((S)-oxetan-2-yl)methyl)-1H- benzo[d]imidazole-6-carboxylic acid |
| 141-P2 | 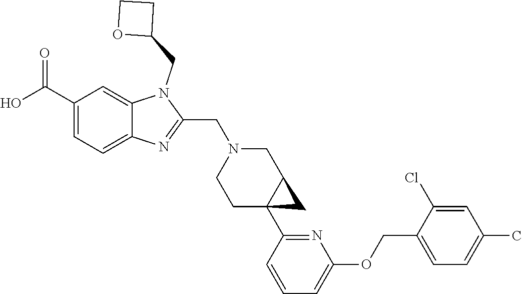 [SMILES: Cc1ccc(COc2cccc([C@]34CCN(Cc5nc6ccc(C(=O)O)cc6n5C[C@@H]5CCO5)C[C@H]3C4)n2)c(Cl)c1] | 2-(((1S,6R)-6-(6-((2,4- dichlorobenzyl)oxy)pyridin-2-yl)-3- azabicyclo[4.1.0]heptan-3-yl)methyl)-1- (((S)-oxetan-2-yl)methyl)-1H- benzo[d]imidazole-6-carboxylic acid |
| 142 | 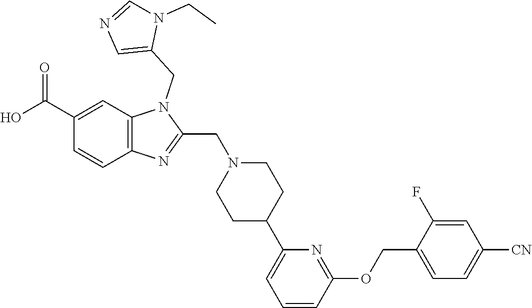 [SMILES: CCn1cncc1Cn1c(CN2CCC(c3cccc(OCc4ccc(C#N)cc4F)n3)CC2)nc2ccc(C(=O)O)cc21] | 2-((4-(6-((4-cyano-2- fluorobenzyl)oxy)pyridin-2-yl)piperidin-1- yl)methyl)-1-((1-ethyl-1H-imidazol-5- yl)methyl)-1H-benzo[d]imidazole-6- carboxylic acid |
| 143 | 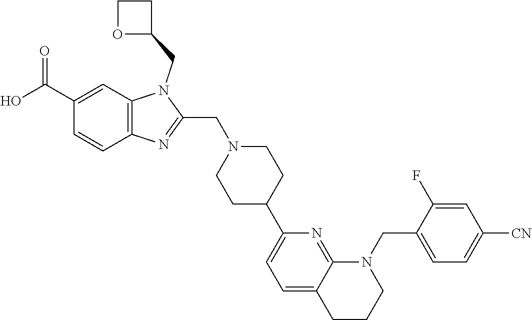 [SMILES: N#Cc1ccc(CN2CCCc3ccc(C4CCN(Cc5nc6ccc(C(=O)O)cc6n5C[C@@H]5CCO5)CC4)nc32)c(F)c1] | (S)-2-((4-(8-(4-cyano-2-fluorobenzyl)- 5,6,7,8-tetrahydro-1,8-naphthyridin-2- yl)piperidin-1-yl)methyl)-1-(oxetan-2- ylmethyl)-1H-benzo[d]imidazole-6- carboxylic acid |
| 144 | 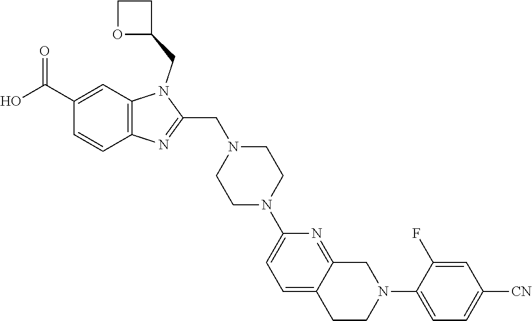 [SMILES: N#Cc1ccc(N2CCc3ccc(N4CCN(Cc5nc6ccc(C(=O)O)cc6n5C[C@@H]5CCO5)CC4)nc3C2)c(F)c1] | (S)-2-((4-(7-(4-cyano-2-fluorophenyl)- 5,6,7,8-tetrahydro-1,7-naphthyridin-2- yl)piperazin-1-yl)methyl)-1-(oxetan-2- ylmethyl)-1H-benzo[d]imidazole-6- carboxylic acid |
| 145 | 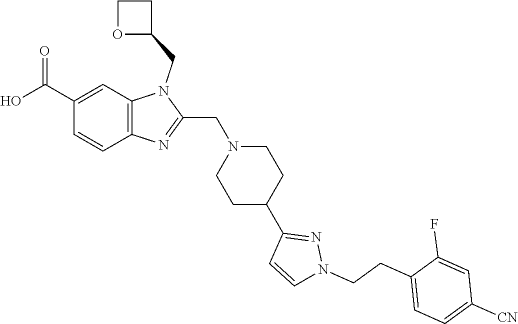 [SMILES: N#Cc1ccc(CCn2ccc(C3CCN(Cc4nc5ccc(C(=O)O)cc5n4C[C@@H]4CCO4)CC3)n2)c(F)c1] | (S)-2-((4-(1-(4-cyano-2-fluorophenethyl)- 1H-pyrazol-3-yl)piperidin-1-yl)methyl)-1- (oxetan-2-ylmethyl)-1H- benzo[d]imidazole-6-carboxylic acid |
| 146-P1 | 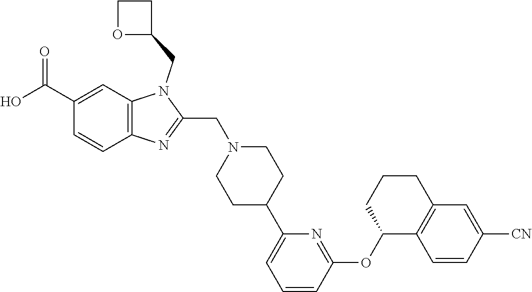 [SMILES: N#Cc1ccc2c(c1)CCC[C@H]2Oc1cccc(C2CCN(Cc3nc4ccc(C(=O)O)cc4n3C[C@@H]3CCO3)CC2)n1] | 2-((4-(6-(((R)-6-cyano-1,2,3,4- tetrahydronaphthalen-1-yl)oxy)pyridin-2- yl)piperidin-1-yl)methyl)-1-(((S)-oxetan- 2-yl)methyl)-1H-benzo[d]imidazole-6- carboxylic acid |
| 146-P2 | 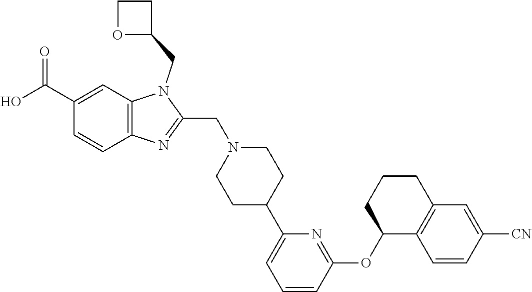 [SMILES: N#Cc1ccc2c(c1)CCC[C@@H]2Oc1cccc(C2CCN(Cc3nc4ccc(C(=O)O)cc4n3C[C@@H]3CCO3)CC2)n1] | 2-((4-(6-(((S)-6-cyano-1,2,3,4- tetrahydronaphthalen-1-yl)oxy)pyridin-2- yl)piperidin-1-yl)methyl)-1-(((S)-oxetan- 2-yl)methyl)-1H-benzo[d]imidazole-6- carboxylic acid |
| 147 | 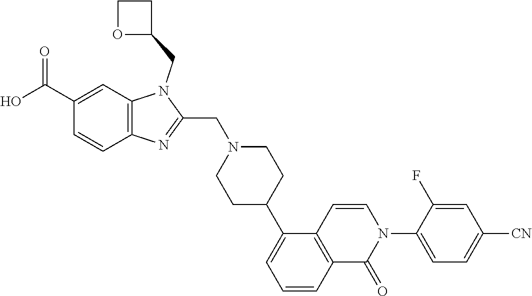 [SMILES: N#Cc1ccc(-n2ccc3c(C4CCN(Cc5nc6ccc(C(=O)O)cc6n5C[C@@H]5CCO5)CC4)cccc3c2=O)c(F)c1] | (S)-2-((4-(2-(4-cyano-2-fluorophenyl)-1- oxo-1,2-dihydroisoquinolin-5- yl)piperidin-1-yl)methyl)-1-(oxetan-2- ylmethyl)-1H-benzo[d]imidazole-6- carboxylic acid |
| 148 | 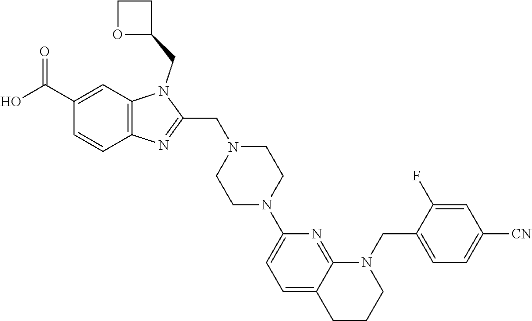 [SMILES: N#Cc1ccc(CN2CCCc3ccc(N4CCN(Cc5nc6ccc(C(=O)O)cc6n5C[C@@H]5CCO5)CC4)nc32)c(F)c1] | (S)-2-((4-(8-(4-cyano-2-fluorobenzyl)- 5,6,7,8-tetrahydro-1,8-naphthyridin-2- yl)piperazin-1-yl)methyl)-1-(oxetan-2- ylmethyl)-1H-benzo[d]imidazole-6- carboxylic acid |
| 149 | 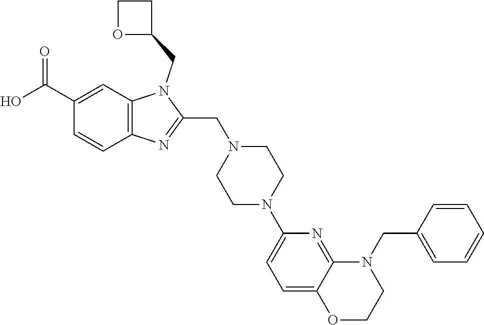 [SMILES: O=C(O)c1ccc2nc(CN3CCN(c4ccc5c(n4)N(Cc4ccccc4)CCO5)CC3)n(C[C@@H]3CCO3)c2c1] | (S)-2-((4-(4-benzyl-3,4-dihydro-2H- pyrido[3,2-b][1,4]oxazin-6-yl)piperazin- 1-yl)methyl)-1-(oxetan-2-ylmethyl)-1H- benzo[d]imidazole-6-carboxylic acid |
| 150 | 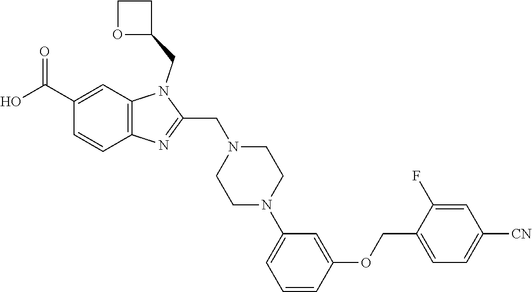 [SMILES: N#Cc1ccc(COc2cccc(N3CCN(Cc4nc5ccc(C(=O)O)cc5n4C[C@@H]4CCO4)CC3)c2)c(F)c1] | (S)-2-((4-(3-((4-cyano-2- fluorobenzyl)oxy)phenyl)piperazin-1- yl)methyl)-1-(oxetan-2-ylmethyl)-1H- benzo[d]imidazole-6-carboxylic acid |
| 151-P1 | 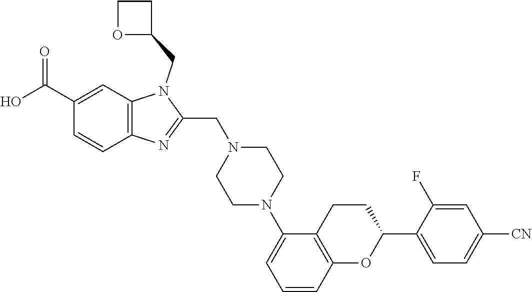 [SMILES: N#Cc1ccc([C@H]2CCc3c(cccc3N3CCN(Cc4nc5ccc(C(=O)O)cc5n4C[C@@H]4CCO4)CC3)O2)c(F)c1] | 2-((4-((R)-2-(4-cyano-2- fluorophenyl)chroman-5-yl)piperazin-1- yl)methyl)-1-(((S)-oxetan-2-yl)methyl)- 1H-benzo[d]imidazole-6-carboxylic acid |
| 151-P2 | 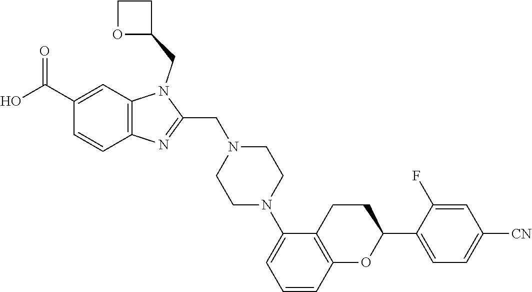 [SMILES: N#Cc1ccc([C@@H]2CCc3c(cccc3N3CCN(Cc4nc5ccc(C(=O)O)cc5n4C[C@@H]4CCO4)CC3)O2)c(F)c1] | 2-((4-((S)-2-(4-cyano-2- fluorophenyl)chroman-5-yl)piperazin-1- yl)methyl)-1-(((S)-oxetan-2-yl)methyl)- 1H-benzo[d]imidazole-6-carboxylic acid |
| 152 | 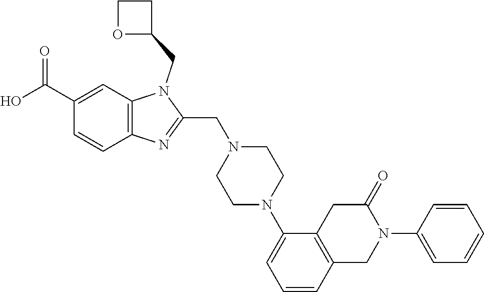 [SMILES: O=C(O)c1ccc2nc(CN3CCN(c4cccc5c4CC(=O)N(c4ccccc4)C5)CC3)n(C[C@@H]3CCO3)c2c1] | (S)-1-(oxetan-2-ylmethyl)-2-((4-(3-oxo-2- phenyl-1,2,3,4-tetrahydroisoquinolin-5- yl)piperidin-1-yl)methyl)-1H- benzo[d]imidazole-6-carboxylic acid |
| 153 | 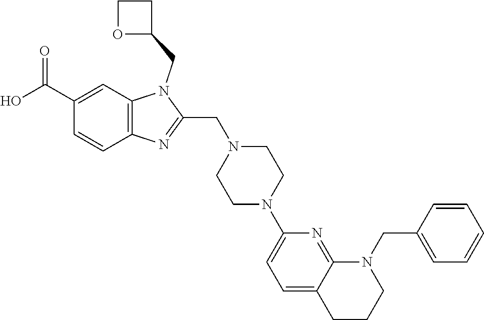 [SMILES: O=C(O)c1ccc2nc(CN3CCN(c4ccc5c(n4)N(Cc4ccccc4)CCC5)CC3)n(C[C@@H]3CCO3)c2c1] | (S)-2-((4-(8-benzyl-5,6,7,8-tetrahydro- 1,8-naphthyridin-2-yl)piperazin-1- yl)methyl)-1-(oxetan-2-ylmethyl)-1H- benzo[d]imidazole-6-carboxylic acid |
| 154 | 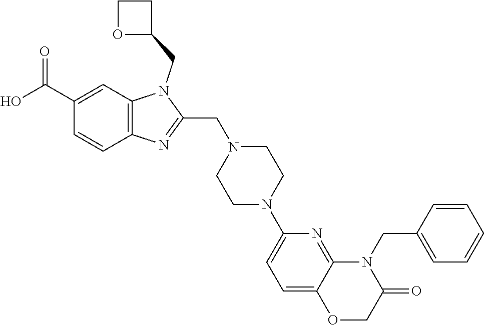 [SMILES: O=C(O)c1ccc2nc(CN3CCN(c4ccc5c(n4)N(Cc4ccccc4)C(=O)CO5)CC3)n(C[C@@H]3CCO3)c2c1] | (S)-2-((4-(4-benzyl-3-oxo-3,4-dihydro- 2H-pyrido[3,2-b][1,4]oxazin-6- yl)piperazin-1-yl)methyl)-1-(oxetan-2- ylmethyl)-1H-benzo[d]imidazole-6- carboxylic acid |
| 155 | 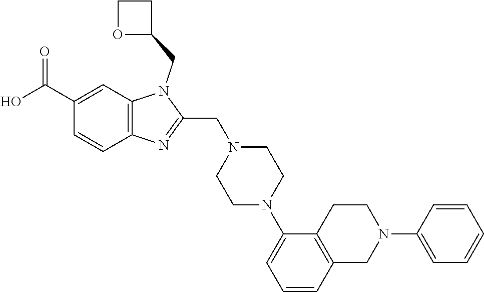 [SMILES: O=C(O)c1ccc2nc(CN3CCN(c4cccc5c4CCN(c4ccccc4)C5)CC3)n(C[C@@H]3CCO3)c2c1] | (S)-1-(oxetan-2-ylmethyl)-2-((4-(2- phenyl-1,2,3,4-tetrahydroisoquinolin-5- yl)piperazin-1-yl)methyl)-1H- benzo[d]imidazole-6-carboxylic acid |
| 156 | 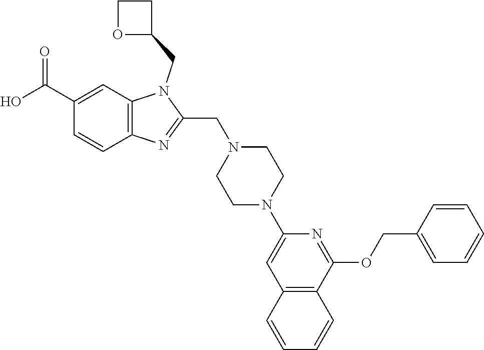 [SMILES: O=C(O)c1ccc2nc(CN3CCN(c4cc5ccccc5c(OCc5ccccc5)n4)CC3)n(C[C@@H]3CCO3)c2c1] | (S)-2-((4-(1-(benzyloxy)isoquinolin-3- yl)piperazin-1-yl)methyl)-1-(oxetan-2- ylmethyl)-1H-benzo[d]imidazole-6- carboxylic acid |
| 157 | 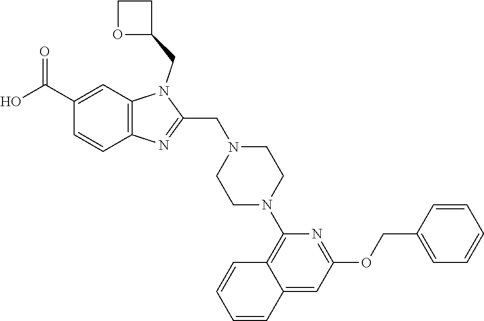 [SMILES: O=C(O)c1ccc2nc(CN3CCN(c4nc(OCc5ccccc5)cc5ccccc45)CC3)n(C[C@@H]3CCO3)c2c1] | (S)-2-((4-(3-(benzyloxy)isoquinolin-1- yl)piperazin-1-yl)methyl)-1-(oxetan-2- ylmethyl)-1H-benzo[d]imidazole-6- carboxylic acid |
| 158 | 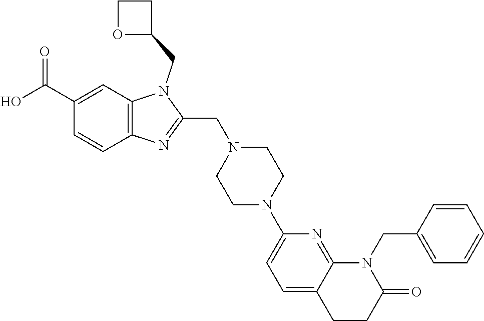 [SMILES: O=C(O)c1ccc2nc(CN3CCN(c4ccc5c(n4)N(Cc4ccccc4)C(=O)CC5)CC3)n(C[C@@H]3CCO3)c2c1] | (S)-2-((4-(8-benzyl-7-oxo-5,6,7,8- tetrahydro-1,8-naphthyridin-2- yl)piperazin-1-yl)methyl)-1-(oxetan-2- ylmethyl)-1H-benzo[d]imidazole-6- carboxylic acid |
| 159 | 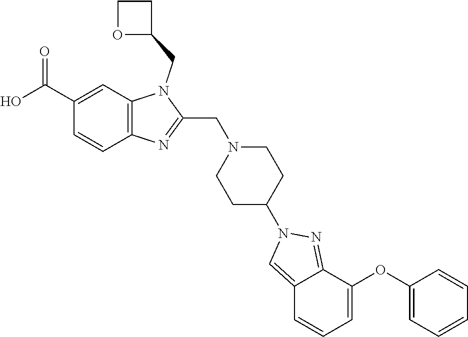 [SMILES: O=C(O)c1ccc2nc(CN3CCC(n4cc5cccc(Oc6ccccc6)c5n4)CC3)n(C[C@@H]3CCO3)c2c1] | (S)-1-(oxetan-2-ylmethyl)-2-((4-(7- phenoxy-2H-indazol-2-yl)piperidin-1- yl)methyl)-1H-benzo[d]imidazole-6- carboxylic acid |
| 160 | 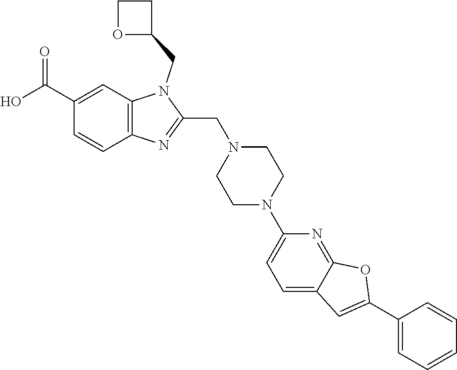 [SMILES: O=C(O)c1ccc2nc(CN3CCN(c4ccc5cc(-c6ccccc6)oc5n4)CC3)n(C[C@@H]3CCO3)c2c1] | (S)-1-(oxetan-2-ylmethyl)-2-((4-(2- phenylfuro[2,3-b]pyridin-6-yl)piperazin- 1-yl)methyl)-1H-benzo[d]imidazole-6- carboxylic acid |
| 161 | 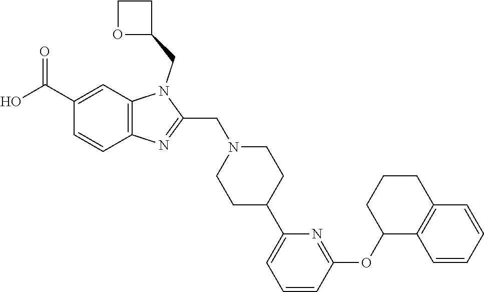 [SMILES: O=C(O)c1ccc2nc(CN3CCC(c4cccc(OC5CCCc6ccccc65)n4)CC3)n(C[C@@H]3CCO3)c2c1] | 1-(((S)-oxetan-2-yl)methyl)-2-((4-(6- ((1,2,3,4-tetrahydronaphthalen-1- yl)oxy)pyridin-2-yl)piperidin-1- yl)methyl)-1H-benzo[d]imidazole-6- carboxylic acid |
| 162 | 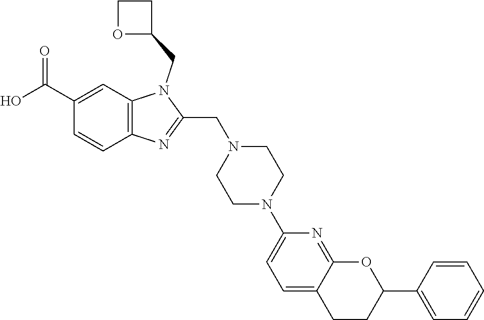 [SMILES: O=C(O)c1ccc2nc(CN3CCN(c4ccc5c(n4)OC(c4ccccc4)CC5)CC3)n(C[C@@H]3CCO3)c2c1] | 1-(((S)-oxetan-2-yl)methyl)-2-((4-(2- phenyl-3,4-dihydro-2H-pyrano[2,3- b]pyridin-7-yl)piperazin-1-yl)methyl)-1H- benzo[d]imidazole-6-carboxylic acid |
| 163-P1 | 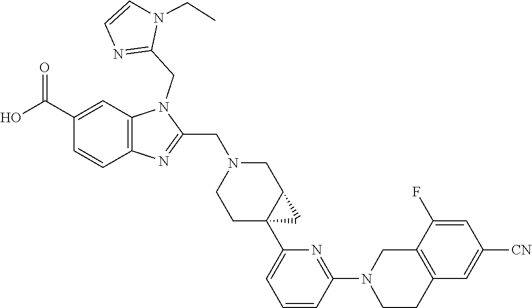 [SMILES: CCn1ccnc1Cn1c(CN2CC[C@@]3(c4cccc(N5CCc6cc(C#N)cc(F)c6C5)n4)C[C@H]3C2)nc2ccc(C(=O)O)cc21] | 2-(((1R,6S)-6-(6-(6-cyano-8-fluoro-3,4- dihydroisoquinolin-2(1H)-yl)pyridin-2- yl)-3-azabicyclo[4.1.0]heptan-3- yl)methyl)-1-((1-ethyl-1H-imidazol-2- yl)methyl)-1H-benzo[d]imidazole-6- carboxylic acid |
| 163-P2 | 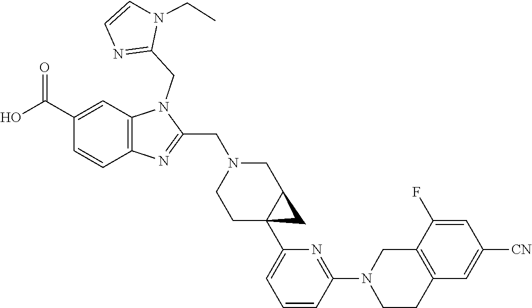 [SMILES: CCn1ccnc1Cn1c(CN2CC[C@]3(c4cccc(N5CCc6cc(C#N)cc(F)c6C5)n4)C[C@@H]3C2)nc2ccc(C(=O)O)cc21] | 2-(((1S,6R)-6-(6-(6-cyano-8-fluoro-3,4- dihydroisoquinolin-2(1H)-yl)pyridin-2- yl)-3-azabicyclo[4.1.0]heptan-3- yl)methyl)-1-((1-ethyl-1H-imidazol-2- yl)methyl)-1H-benzo[d]imidazole-6- carboxylic acid |
| 164 | 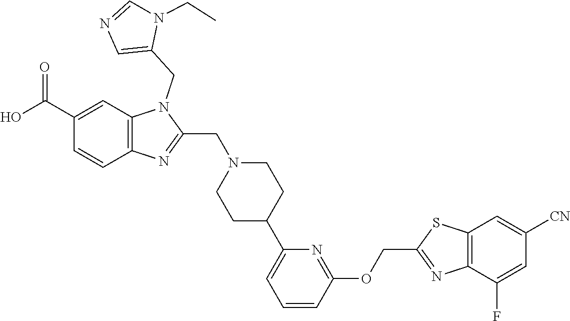 [SMILES: CCn1cncc1Cn1c(CN2CCC(c3cccc(OCc4nc5c(F)cc(C#N)cc5s4)n3)CC2)nc2ccc(C(=O)O)cc21] | 2-((4-(6-((6-cyano-4- fluorobenzo[d]thiazol-2- yl)methoxy)pyridin-2-yl)piperidin-1- yl)methyl)-1-((1-ethyl-1H-imidazol-5- yl)methyl)-1H-benzo[d]imidazole-6- carboxylic acid |
| 165 | 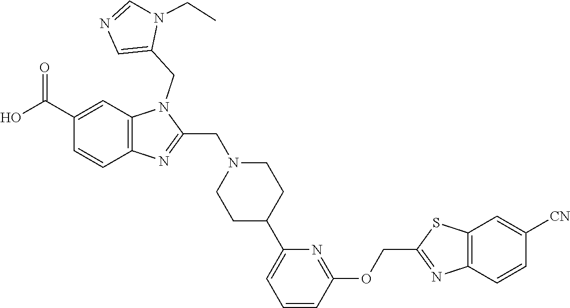 [SMILES: CCn1cncc1Cn1c(CN2CCC(c3cccc(OCc4nc5ccc(C#N)cc5s4)n3)CC2)nc2ccc(C(=O)O)cc21] | 2-((4-(6-((6-cyanobenzo[d]thiazol-2- yl)methoxy)pyridin-2-yl)piperidin-1- yl)methyl)-1-((1-ethyl-1H-imidazol-5- yl)methyl)-1H-benzo[d]imidazole-6- carboxylic acid |
| 166 | 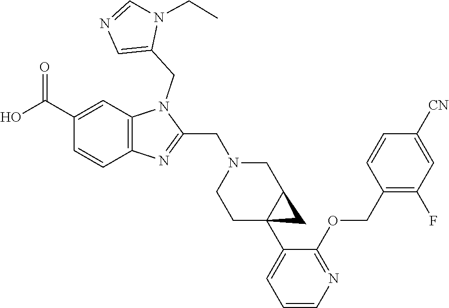 [SMILES: CCn1cncc1Cn1c(CN2CC[C@]3(c4cccnc4OCc4ccc(C#N)cc4F)C[C@@H]3C2)nc2ccc(C(=O)O)cc21] | 2-((6-(2-((4-cyano-2- fluorobenzyl)oxy)pyridin-3-yl)-3- azabicyclo[4.1.0]heptan-3-yl)methyl)-1- ((1-ethyl-1H-imidazol-5-yl)methyl)-1H- benzo[d]imidazole-6-carboxylic acid |
| 167 | 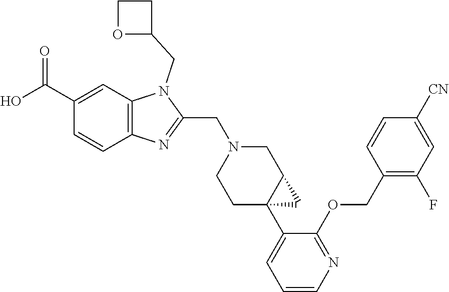 [SMILES: N#Cc1ccc(COc2ncccc2[C@@]23CCN(Cc4nc5ccc(C(=O)O)cc5n4CC4CCO4)C[C@@H]2C3)c(F)c1] | 2-((6-(2-((4-cyano-2- fluorobenzyl)oxy)pyridin-3-yl)-3- azabicyclo[4.1.0]heptan-3-yl)methyl)-1- (oxetan-2-ylmethyl)-1H- benzo[d]imidazole-6-carboxylic acid |
| 168 | 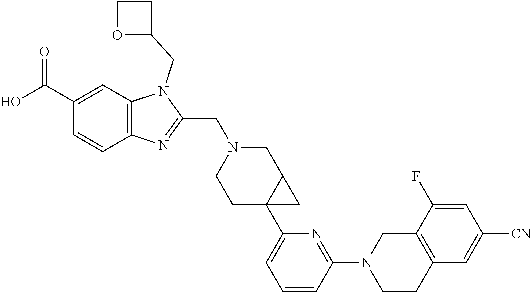 [SMILES: N#Cc1cc(F)c2c(c1)CCN(c1cccc(C34CCN(Cc5nc6ccc(C(=O)O)cc6n5CC5CCO5)CC3C4)n1)C2] | 2-((6-(6-(6-cyano-8-fluoro-3,4- dihydroisoquinolin-2(1H)-yl)pyridin-2- yl)-3-azabicyclo[4.1.0]heptan-3- yl)methyl)-1-(oxetan-2-ylmethyl)-1H- benzo[d]imidazole-6-carboxylic acid |
| 169 | 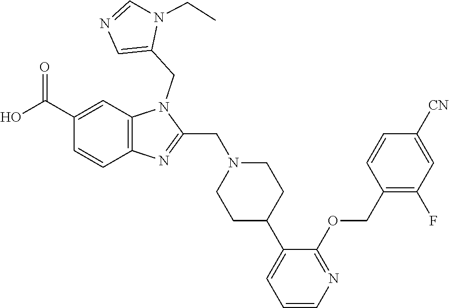 [SMILES: CCn1cncc1Cn1c(CN2CCC(c3cccnc3OCc3ccc(C#N)cc3F)CC2)nc2ccc(C(=O)O)cc21] | 2-((4-(2-((4-cyano-2- fluorobenzyl)oxy)pyridin-3-yl)piperidin- 1-yl)methyl)-1-((1-ethyl-1H-imidazol-5- yl)methyl)-1H-benzo[d]imidazole-6- carboxylic acid |
| 170 | 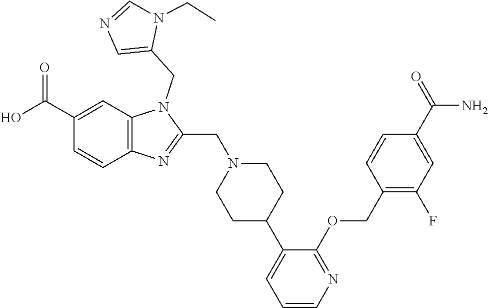 [SMILES: CCn1cncc1Cn1c(CN2CCC(c3cccnc3OCc3ccc(C(N)=O)cc3F)CC2)nc2ccc(C(=O)O)cc21] | 2-((4-(2-((4-carbamoyl-2- fluorobenzyl)oxy)pyridin-3-yl)piperidin- 1-yl)methyl)-1-((1-ethyl-1H-imidazol-5- yl)methyl)-1H-benzo[d]imidazole-6- carboxylic acid |
| 171 | 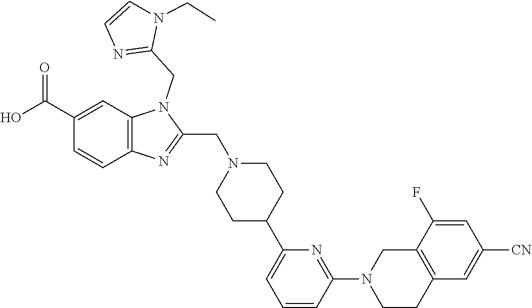 [SMILES: CCn1ccnc1Cn1c(CN2CCC(c3cccc(N4CCc5cc(C#N)cc(F)c5C4)n3)CC2)nc2ccc(C(=O)O)cc21] | 2-((4-(6-(6-cyano-8-fluoro-3,4- dihydroisoquinolin-2(1H)-yl)pyridin-2- yl)piperidin-1-yl)methyl)-1-((1-ethyl-1H- imidazol-2-yl)methyl)-1H- benzo[d]imidazole-6-carboxylic acid |
| 172 | 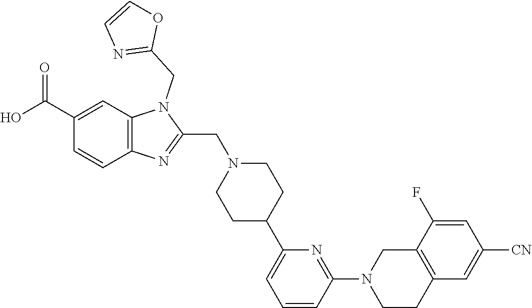 [SMILES: N#Cc1cc(F)c2c(c1)CCN(c1cccc(C3CCN(Cc4nc5ccc(C(=O)O)cc5n4Cc4ncco4)CC3)n1)C2] | 2-((4-(6-(6-cyano-8-fluoro-3,4- dihydroisoquinolin-2(1H)-yl)pyridin-2- yl)piperidin-1-yl)methyl)-1-(oxazol-2- ylmethyl)-1H-benzo[d]imidazole-6- carboxylic acid |
| 173 | 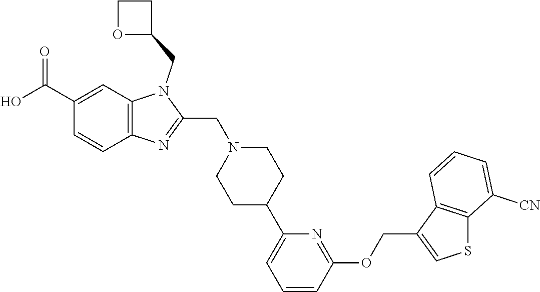 [SMILES: N#Cc1cccc2c(COc3cccc(C4CCN(Cc5nc6ccc(C(=O)O)cc6n5C[C@@H]5CCO5)CC4)n3)csc12] | (S)-2-((4-(6-((7-cyanobenzo[b]thiophen- 3-yl)methoxy)pyridin-2-yl)piperidin-1- yl)methyl)-1-(oxetan-2-ylmethyl)-1H- benzo[d]imidazole-6-carboxylic acid |
| 174 | 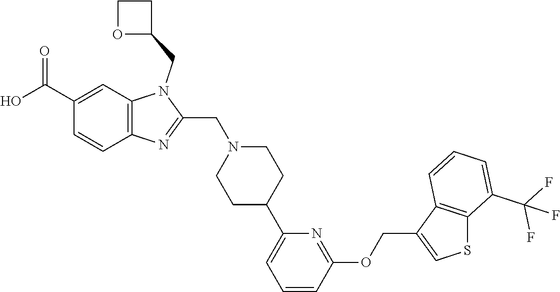 [SMILES: O=C(O)c1ccc2nc(CN3CCC(c4cccc(OCc5csc6c(C(F)(F)F)cccc56)n4)CC3)n(C[C@@H]3CCO3)c2c1] | (S)-1-(oxetan-2-ylmethyl)-2-((4-(6-((7- (trifluoromethyl)benzo[b]thiophen-3- yl)methoxy)pyridin-2-yl)piperidin-1- yl)methyl)-1H-benzo[d]imidazole-6- carboxylic acid |
| 175 | 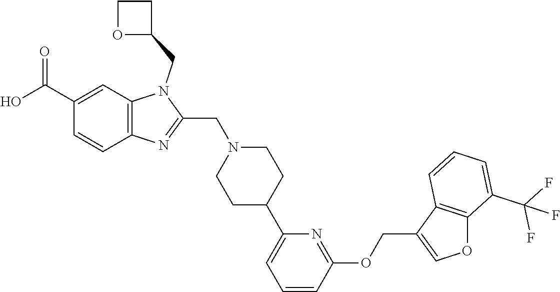 [SMILES: O=C(O)c1ccc2nc(CN3CCC(c4cccc(OCc5coc6c(C(F)(F)F)cccc56)n4)CC3)n(C[C@@H]3CCO3)c2c1] | (S)-1-(oxetan-2-ylmethyl)-2-((4-(6-((7- (trifluoromethyl)benzofuran-3- yl)methoxy)pyridin-2-yl)piperidin-1- yl)methyl)-1H-benzo[d]imidazole-6- carboxylic acid |
| 176 | 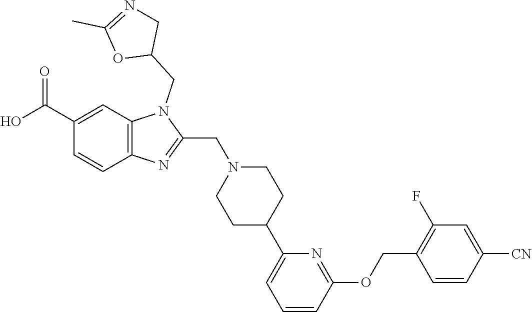 [SMILES: CC1=NCC(Cn2c(CN3CCC(c4cccc(OCc5ccc(C#N)cc5F)n4)CC3)nc3ccc(C(=O)O)cc32)O1] | 2-((4-(6-((4-cyano-2- fluorobenzyl)oxy)pyridin-2-yl)piperidin- 1-yl)methyl)-1-((2-methyl-4,5- dihydrooxazol-5-yl)methyl)-1H- benzo[d]imidazole-6-carboxylic acid |
| 177 | 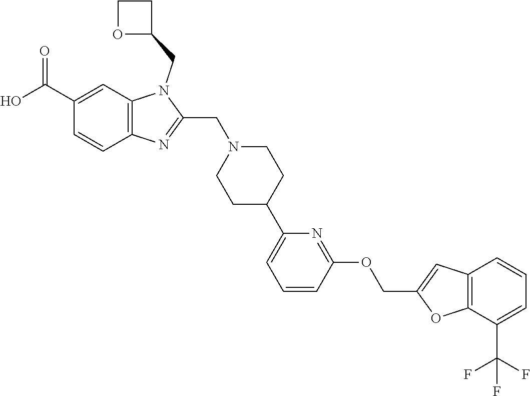 [SMILES: O=C(O)c1ccc2nc(CN3CCC(c4cccc(OCc5cc6cccc(C(F)(F)F)c6o5)n4)CC3)n(C[C@@H]3CCO3)c2c1] | (S)-1-(oxetan-2-ylmethyl)-2-((4-(6-((7- (trifluoromethyl)benzofuran-2- yl)methoxy)pyridin-2-yl)piperidin-1- yl)methyl)-1H-benzo[d]imidazole-6- carboxylic acid |
| 178 | 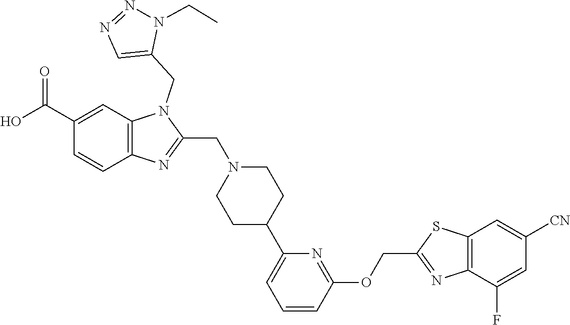 [SMILES: CCn1nncc1Cn1c(CN2CCC(c3cccc(OCc4nc5c(F)cc(C#N)cc5s4)n3)CC2)nc2ccc(C(=O)O)cc21] | 2-((4-(6-((6-cyano-4- fluorobenzo[d]thiazol-2- yl)methoxy)pyridin-2-yl)piperidin-1- yl)methyl)-1-((1-ethyl-1H-1,2,3-triazol-5- yl)methyl)-1H-benzo[d]imidazole-6- carboxylic acid |
| 179 | 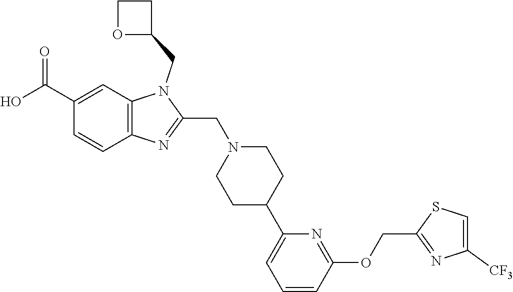 [SMILES: O=C(O)c1ccc2nc(CN3CCC(c4cccc(OCc5nc(C(F)(F)F)cs5)n4)CC3)n(C[C@@H]3CCO3)c2c1] | (S)-1-(oxetan-2-ylmethyl)-2-((4-(6-((4- (trifluoromethyl)thiazol-2- yl)methoxy)pyridin-2-yl)piperidin-1- yl)methyl)-1H-benzo[d]imidazole-6- carboxylic acid |
| 180 | 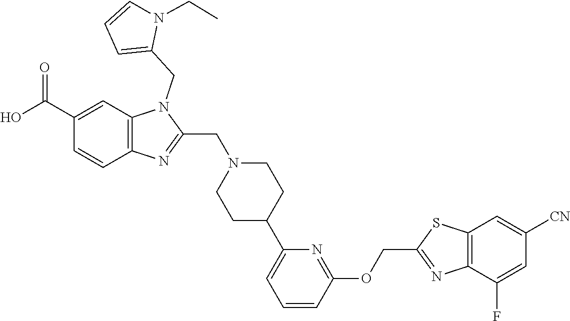 [SMILES: CCn1cccc1Cn1c(CN2CCC(c3cccc(OCc4nc5c(F)cc(C#N)cc5s4)n3)CC2)nc2ccc(C(=O)O)cc21] | 2-((4-(6-((6-cyano-4- fluorobenzo[d]thiazol-2- yl)methoxy)pyridin-2-yl)piperidin-1- yl)methyl)-1-((1-ethyl-1H-pyrrol-2- yl)methyl)-1H-benzo[d]imidazole-6- carboxylic acid |
| 181 | 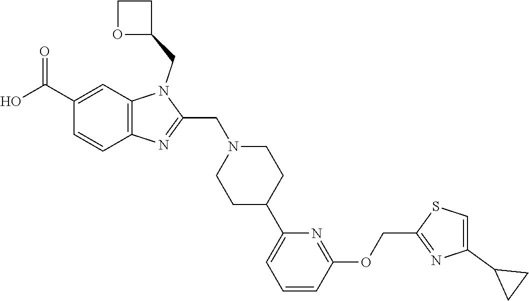 [SMILES: O=C(O)c1ccc2nc(CN3CCC(c4cccc(OCc5nc(C6CC6)cs5)n4)CC3)n(C[C@@H]3CCO3)c2c1] | (S)-2-((4-(6-((4-cyclopropylthiazol-2- yl)methoxy)pyridin-2-yl)piperidin-1- yl)methyl)-1-(oxetan-2-ylmethyl)-1H- benzo[d]imidazole-6-carboxylic acid |
| 182 | 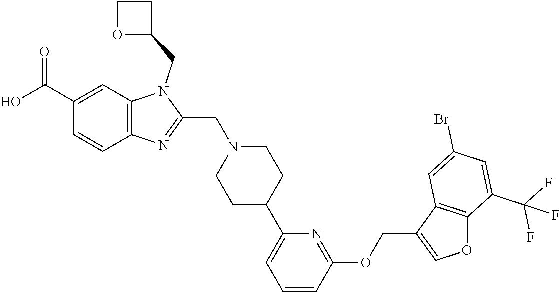 [SMILES: O=C(O)c1ccc2nc(CN3CCC(c4cccc(OCc5coc6c(C(F)(F)F)cc(Br)cc56)n4)CC3)n(C[C@@H]3CCO3)c2c1] | (S)-2-((4-(6-((5-bromo-7- (trifluoromethyl)benzofuran-3- yl)methoxy)pyridin-2-yl)piperidin-1- yl)methyl)-1-(oxetan-2-ylmethyl)-1H- benzo[d]imidazole-6-carboxylic acid |
| 183 | 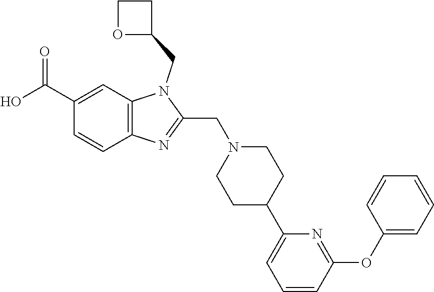 [SMILES: O=C(O)c1ccc2nc(CN3CCC(c4cccc(Oc5ccccc5)n4)CC3)n(C[C@@H]3CCO3)c2c1] | (S)-1-(oxetan-2-ylmethyl)-2-((4-(6- phenoxypyridin-2-yl)piperidin-1- yl)methyl)-1H-benzo[d]imidazole-6- carboxylic acid |
| 184-P1 | 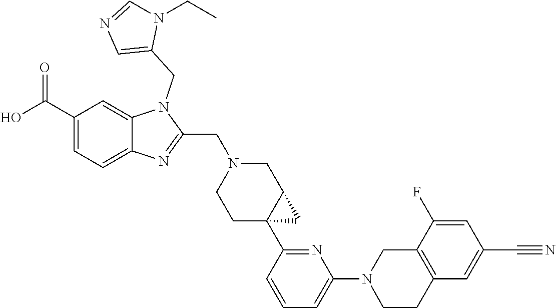 [SMILES: CCn1cncc1Cn1c(CN2CC[C@@]3(c4cccc(N5CCc6cc(C#N)cc(F)c6C5)n4)C[C@H]3C2)nc2ccc(C(=O)O)cc21] | 2-(((1R,6S)-6-(6-(6-cyano-8-fluoro-3,4- dihydroisoquinolin-2(1H)-yl)pyridin-2- yl)-3-azabicyclo[4.1.0]heptan-3- yl)methyl)-1-((1-ethyl-1H-imidazol-5- yl)methyl)-1H-benzo[d]imidazole-6- carboxylic acid |
| 184-P2 | 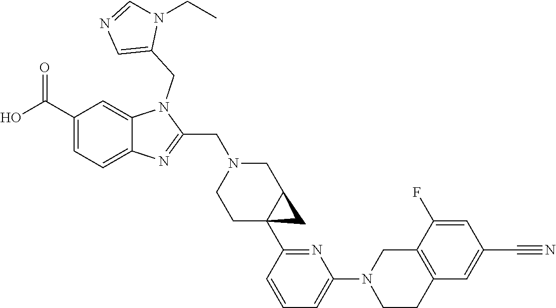 [SMILES: CCn1cncc1Cn1c(CN2CC[C@]3(c4cccc(N5CCc6cc(C#N)cc(F)c6C5)n4)C[C@@H]3C2)nc2ccc(C(=O)O)cc21] | 2-(((1S,6R)-6-(6-(6-cyano-8-fluoro-3,4- dihydroisoquinolin-2(1H)-yl)pyridin-2- yl)-3-azabicyclo[4.1.0]heptan-3- yl)methyl)-1-((1-ethyl-1H-imidazol-5- yl)methyl)-1H-benzo[d]imidazole-6- carboxylic acid |
| 185-P1 | 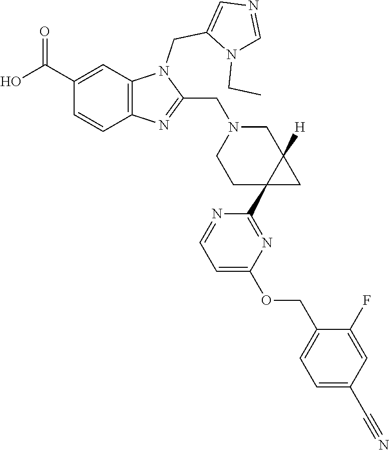 [SMILES: CCn1cncc1Cn1c(CN2CC[C@@]3(c4nccc(OCc5ccc(C#N)cc5F)n4)C[C@H]3C2)nc2ccc(C(=O)O)cc21] | 2-(((1R,6S)-6-(4-((4-cyano-2- fluorobenzyl)oxy)pyrimidin-2-yl)-3- azabicyclo[4.1.0]heptan-3-yl)methyl)-1- ((1-ethyl-1H-imidazol-5-yl)methyl)-1H- benzo[d]imidazole-6-carboxylic acid |
| 185-P2 | 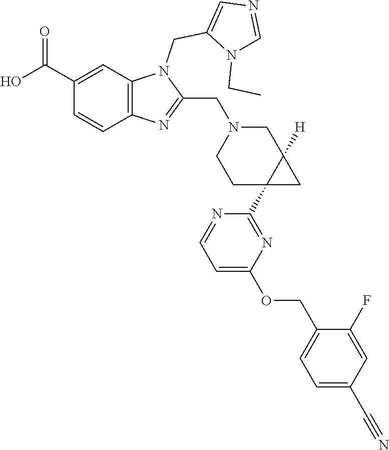 [SMILES: CCn1cncc1Cn1c(CN2CC[C@]3(c4nccc(OCc5ccc(C#N)cc5F)n4)C[C@@H]3C2)nc2ccc(C(=O)O)cc21] | 2-(((1S,6R)-6-(4-((4-cyano-2- fluorobenzyl)oxy)pyrimidin-2-yl)-3- azabicyclo[4.1.0]heptan-3-yl)methyl)-1- ((1-ethyl-1H-imidazol-5-yl)methyl)-1H- benzo[d]imidazole-6-carboxylic acid |
| 186-P1 | 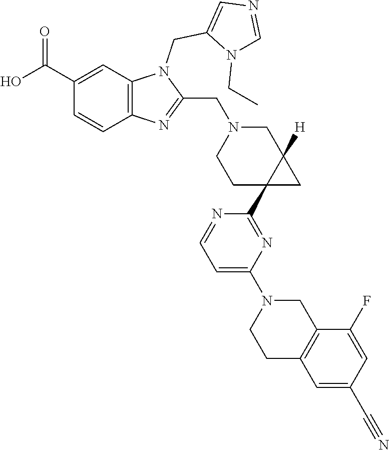 [SMILES: CCn1cncc1Cn1c(CN2CC[C@@]3(c4nccc(N5CCc6cc(C#N)cc(F)c6C5)n4)C[C@H]3C2)nc2ccc(C(=O)O)cc21] | 2-(((1S,6R)-6-(4-((4-cyano-2- fluorobenzyl)oxy)pyrimidin-2-yl)-3- azabicyclo[4.1.0]heptan-3-yl)methyl)-1- ((1-ethyl-1H-imidazol-5-yl)methyl)-1H- benzo[d]imidazole-6-carboxylic acid |
| 186-P2 | 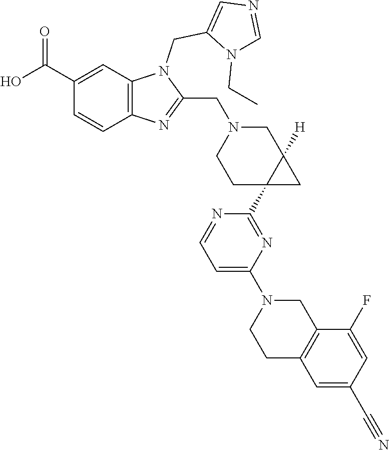 [SMILES: CCn1cncc1Cn1c(CN2CC[C@]3(c4nccc(N5CCc6cc(C#N)cc(F)c6C5)n4)C[C@@H]3C2)nc2ccc(C(=O)O)cc21] | 2-(((1R,6S)-6-(4-(6-cyano-8-fluoro-3,4- dihydroisoquinolin-2(1H)-yl)pyrimidin-2- yl)-3-azabicyclo[4.1.0]heptan-3- yl)methyl)-1-((1-ethyl-1H-imidazol-5- yl)methyl)-1H-benzo[d]imidazole-6- carboxylic acid |
| 187 | 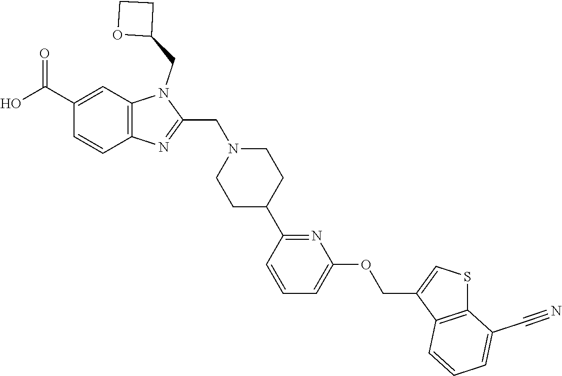 [SMILES: N#Cc1cccc2c(COc3cccc(C4CCN(Cc5nc6ccc(C(=O)O)cc6n5C[C@@H]5CCO5)CC4)n3)csc12] | (S)-2-((4-(6-((7-cyanobenzo[b]thiophen- 3-yl)methoxy)pyridin-2-yl)piperidin-1- yl)methyl)-1-(oxetan-2-ylmethyl)-1H- benzo[d]imidazole-6-carboxylic acid |

In another aspect, provided is a method of making a compound of Formula (I), including compounds of Formulae (II)-(IV), or selected from the group consisting of a compound listed in Table 1, or a stereoisomer, tautomer, or a pharmaceutically acceptable salt of any of the foregoing. Compounds described herein may be prepared according to general schemes, as exemplified by the general procedures and examples. Minor variations in temperatures, concentrations, reaction times, and other parameters can be made when following the general procedures, which do not substantially affect the results of the procedures.

Also provided are compound intermediates useful in synthesis of a compound of Formula (I), including compounds of Formulae (II)-(IV), or selected from the group consisting of a compound listed in Table 1, or a stereoisomer, tautomer, or a pharmaceutically acceptable salt of any of the foregoing. Synthesis of representative compounds and intermediates are shown in the examples below.

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described. Compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio, unless a specific stereochemistry is otherwise indicated. Where a compound of Table 1 is depicted with a particular stereochemical configuration, also provided herein is any alternative stereochemical configuration of the compound, as well as a mixture of stereoisomers of the compound in any ratio. For example, where a compound of Table 1 has a stereocenter that is in an “S” stereochemical configuration, also provided herein is the enantiomer of the compound wherein that stereocenter is in an “R” stereochemical configuration. Likewise, when a compound of Table 1 has a stereocenter that is in an “R” configuration, also provided herein is enantiomer of the compound in an “S” stereochemical configuration. Also provided are mixtures of the compound with both the “S” and the “R” stereochemical configuration.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, 3H, ¹¹C, ¹³C, 1C, ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution study. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

The invention also includes any or all metabolites of any of the compounds described. The metabolites may include any chemical species generated by a biotransformation of any of the compounds described, such as intermediates and products of metabolism of the compound, such as would be generated in vivo following administration to a human.

### Pharmaceutically Acceptable Compositions and Formulations

Pharmaceutically acceptable compositions or simply “pharmaceutical compositions” of any of the compounds detailed herein are embraced by this invention. Thus, the invention includes pharmaceutical compositions comprising a compound of Formula (I) (including compounds of Formulae (II)-(IV)), or a stereoisomer, tautomer, or a pharmaceutically acceptable salt of any of the foregoing, and a pharmaceutically acceptable carrier or excipient.

In some embodiments, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound intends a composition that contains no more than 35% impurity, wherein the impurity denotes a compound other than the compound or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form.

In one variation, the compounds herein are synthetic compounds prepared for administration to an individual such as a human. In another variation, compositions are provided containing a compound in substantially pure form. In another variation, the invention embraces pharmaceutical compositions comprising a compound detailed herein and a pharmaceutically acceptable carrier or excipient. In another variation, methods of administering a compound are provided. The purified forms, pharmaceutical compositions and methods of administering the compounds are suitable for any compound or form thereof detailed herein.

The compounds may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Compounds described herein can be used in the preparation of a formulation, such as a pharmaceutical formulation, by combining the compounds as active ingredients with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21st ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Compositions comprising two compounds utilized herein are described. Any of the compounds described herein can be formulated in a tablet in any dosage form described herein. In some embodiments, the composition comprises a compound of Formula (I) (including compounds of Formulae (II)-(IV)), or a stereoisomer, tautomer, or a pharmaceutically acceptable salt of any of the foregoing, as described herein. In some embodiments, provided herein is a dosage form comprises a therapeutically effective amount of a compound of Formula (I) (including compounds of Formulae (II)-(IV)), or a stereoisomer, tautomer, or a pharmaceutically acceptable salt of any of the foregoing. In some embodiments, the compound or a stereoisomer, tautomer, or a pharmaceutically acceptable salt of any of the foregoing is selected from Compound Nos. 1-142 in Table 1. In some embodiments, the compound or a stereoisomer, tautomer, or a pharmaceutically acceptable salt of any of the foregoing is selected from Compound Nos. 143-187 in Table 1. In some embodiments, the compound or a stereoisomer, tautomer, or a pharmaceutically acceptable salt of any of the foregoing is selected from Compound Nos. 1-187 in Table 1.

### Methods of Use and Uses

Compounds and compositions described herein may in some aspects be used in treatment of diseases and/or conditions described herein, for example, diseases and/or conditions mediated by GLP-1R. In some embodiments, the method of treating a disease or condition in a subject in need thereof comprises administering to the subject a therapeutically effective amount of a compound of Formula (I) (including compounds of Formulae (II)-(IV)), or a stereoisomer, tautomer, or a pharmaceutically acceptable salt of any of the foregoing. In some embodiments, the method of treating a disease or condition in a subject in need thereof comprises administering to the subject a therapeutically effective amount of a compound selected from Compound Nos. 1-142 in Table 1, or a stereoisomer, tautomer, or a pharmaceutically acceptable salt of any of the foregoing. In some embodiments, the method of treating a disease or condition in a subject in need thereof comprises administering to the subject a therapeutically effective amount of a compound selected from Compound Nos. 143-187 in Table 1, or a stereoisomer, tautomer, or a pharmaceutically acceptable salt of any of the foregoing. In some embodiments, the method of treating a disease or condition in a subject in need thereof comprises administering to the subject a therapeutically effective amount of a compound selected from Compound Nos. 1-187 in Table 1, or a stereoisomer, tautomer, or a pharmaceutically acceptable salt of any of the foregoing.

In accordance with the present application, a disease or condition to be treated and/or prevented is selected from the group consisting of cardiometabolic and associated diseases including diabetes (T1 D and/or T2DM, including pre-diabetes), idiopathic T1 D (Type 1 b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease (e.g., acute kidney disorder, tubular dysfunction, proinflammatory changes to the proximal tubules), diabetic retinopathy, adipocyte dysfunction, visceral adipose deposition, sleep apnea, obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain from use of other agents (e.g., from use of steroids and antipsychotics), excessive sugar craving, dyslipidemia (including hyperlipidemia, hypertriglyceridemia, increased total cholesterol, high LDL cholesterol, and low HDL cholesterol), hyperinsulinemia, liver diseases such as NAFLD, steatosis, NASH, fibrosis, cirrhosis, and hepatocellular carcinoma, cardiovascular disease, atherosclerosis (including coronary artery disease), peripheral vascular disease, hypertension, endothelial dysfunction, impaired vascular compliance, congestive heart failure, myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome and addiction (e.g., alcohol and/or drug abuse), prevention or treatment of Polycystic Ovary Syndrome and treatment of addiction (e.g., alcohol and/or drug abuse).

In some embodiments, provided herein is a method of treating a cardiometabolic disease in a subject (e.g., a human patient) in need thereof, comprising administering to the subject a therapeutically effective amount of a compound described herein, or a stereoisomer, tautomer, or a pharmaceutically acceptable salt of any of the foregoing.

In some embodiments, provided herein is a method of treating diabetes in a subject (e.g., a human patient) in need thereof, comprising administering to the subject a therapeutically effective amount of a compound described herein, or a stereoisomer, tautomer, or a pharmaceutically acceptable salt of any of the foregoing. Exemplary diabetes include, but are not limited to, T1 D, T2DM, pre-diabetes, idiopathic T1 D, LADA, EOD, YOAD, MODY, malnutrition-related diabetes, and gestational diabetes.

In some embodiments, provided herein is a method of treating a liver disorder in a subject (e.g., a human patient) in need thereof, comprising administering to the subject a therapeutically effective amount of a compound described herein, or a stereoisomer, tautomer, or a pharmaceutically acceptable salt of any of the foregoing. Exemplary liver disorders include, without limitation, liver inflammation, fibrosis, and steatohepatitis. In some embodiments, the liver disorder is selected from the list consisting of primary biliary cirrhosis (PBC), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, nonalcoholic fatty liver disease (NAFLD), nonalcoholic steatohepatitis (NASH), graft versus host disease, transplant liver regeneration, congenital hepatic fibrosis, choledocholithiasis, granulomatous liver disease, intra- or extrahepatic malignancy, Sjogren's syndrome, sarcoidosis, Wilson's disease, Gaucher's disease, hemochromatosis, and oti-antitrypsin deficiency. In some embodiments, the liver disorder is selected from the list consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the liver disorder is selected from the group consisting of liver fibrosis, alcohol induced fibrosis, steatosis, alcoholic steatosis, NAFLD, and NASH. In one embodiment, the liver disorder is NASH. In another embodiment, the liver disorder is liver inflammation. In another embodiment, the liver disorder is liver fibrosis. In another embodiment, the liver disorder is alcohol induced fibrosis. In another embodiment, the liver disorder is steatosis. In another embodiment, the liver disorder is alcoholic steatosis. In another embodiment, the liver disorder is NAFLD. In one embodiment, the treatment methods provided herein impedes or slows the progression of NAFLD to NASH. In one embodiment, the treatment methods provided herein impedes or slows the progression of NASH. NASH can progress, e.g., to one or more of liver cirrhosis, hepatic cancer, etc. In some embodiments, the liver disorder is NASH. In some embodiments, the patient has had a liver biopsy. In some embodiments, the method further comprising obtaining the results of a liver biopsy.

In accordance with the present application, a compound described herein, or a stereoisomer, tautomer, or a pharmaceutically acceptable salt of any of the foregoing, can be administered by any suitable route in the form of a pharmaceutical composition adapted to such a route, and in a dose effective for the treatment intended. In some embodiments, it is a compound of any embodiment of Formula (I) or selected from the compounds of Table 1, or a stereoisomer, tautomer, or a pharmaceutically acceptable salt of any of the foregoing. The compounds and/or compositions described herein may be administered orally, rectally, vaginally, parenterally, or topically.

In some embodiments, the compounds and/or compositions may be administered orally. Oral administration may involve swallowing, so that the compound enters the gastrointestinal tract, or buccal or sublingual administration may be employed by which the compound enters the bloodstream directly from the mouth.

In some embodiments, the compounds and/or compositions may be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In some embodiments, the compounds and/or compositions may be administered topically to the skin or mucosa, that is, dermally or transdermally. In some embodiments, the compounds and/or compositions may be administered intranasally or by inhalation. In some embodiments, the compounds and/or compositions may be administered rectally or vaginally. In some embodiments, the compounds and/or compositions may be administered directly to the eye or ear.

The dosage regimen for the compounds and/or compositions described herein is based on a variety of factors, including the type, age, weight, sex and medical condition of the patient; the severity of the condition; the route of administration; and the activity of the particular compound employed. Thus the dosage regimen may vary widely. In some embodiments, the total daily dose of the compounds of the present application is typically from about 0.001 to about 100 mg/kg (i.e., mg compound per kg body weight) for the treatment of the indicated conditions discussed herein. In one embodiment, total daily dose of the compounds of the present application is from about 0.01 to about 30 mg/kg, and in another embodiment, from about 0.03 to about 10 mg/kg, and in yet another embodiment, from about 0.1 to about 3. It is not uncommon that the administration of the compounds of the present application will be repeated a plurality of times in a day (typically no greater than 4 times). Multiple doses per day typically may be used to increase the total daily dose, if desired.

For oral administration, the compounds and/or compositions described herein may be provided in the form of tablets containing 0.1, 0.5, 10.0, 2.5, 5.0, 10.0, 15.0, 25.0, 30.0 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.01 to about 10 mg/kg/minute during a constant rate infusion.

The compounds and/or compositions described herein can be used alone, or in combination with other therapeutic agents. The administration of two or more agents “in combination” means that all of the agents are administered closely enough in time that each may generate a biological effect in the same time frame. The presence of one agent may alter the biological effects of the other agent(s). The two or more agents may be administered simultaneously, concurrently or sequentially. Additionally, simultaneous administration may be carried out by mixing the agents prior to administration or by administering the compounds at the same point in time but as separate dosage forms at the same or different site of administration.

The present application provides any of the uses, methods or compositions as defined herein wherein a compound of any embodiment of Formula (I) or selected from the compounds of Table 1 as described herein, or a stereoisomer, tautomer, or a pharmaceutically acceptable salt of any of the foregoing, is used in combination with one or more other therapeutic agent. This would include a pharmaceutical composition comprising a compound of any embodiment of Formula (I) or selected from the compounds of Table 1, or a stereoisomer, tautomer, or a pharmaceutically acceptable salt of any of the foregoing, as defined in any of the embodiments described herein, in admixture with at least one pharmaceutically acceptable excipient and one or more other therapeutic agent.

In some embodiments, the one or more other therapeutic agent is an anti-diabetic agent including but not limited to a biguanide (e.g., metformin), a sulfonylurea (e.g., tolbutamide, glibenclamide, gliclazide, chlorpropamide, tolazamide, acetohexamide.glyclopyramide, glimepiride, or glipizide), a thiazolidinedione (e.g., pioglitazone, rosiglitazone, or lobeglitazone), a glitazar (e.g., saroglitazar, aleglitazar, muraglitazar or tesaglitazar), a meglitinide (e.g., nateglinide, repaglinide), a dipeptidyl peptidase 4 (DPP-4) inhibitor (e.g., sitagliptin, vildagliptin, saxagliptin, linagliptin, gemigliptin, anagliptin, teneligliptin, alogliptin, trelagliptin, dutogliptin, or omarigliptin), a glitazone (e.g., pioglitazone, rosiglitazone, balaglitazone, rivoglitazone, or lobeglitazone), a sodium-glucose linked transporter 2 (SGLT2) inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL1 inhibitor, a GPR40 agonist (FFAR1/FFA1 agonist, e.g. fasiglifam), glucose-dependent insulinotropic peptide (GIP) and analogues thereof, an alpha glucosidase inhibitor (e.g. voglibose, acarbose, or miglitol), or an insulin or an insulin analogue, including the pharmaceutically acceptable salts of the specifically named agents and the pharmaceutically acceptable solvates of said agents and salts.

In some embodiments, the one or more other therapeutic agent is an antiobesity agent including but not limited to peptide YY or an analogue thereof, a neuropeptide Y receptor type 2 (NPYR2) agonist, a NPYR1 or NPYR5 antagonist, a cannabinoid receptor type 1 (CB1 R) antagonist, a lipase inhibitor (e.g., orlistat), a human proislet peptide (HIP), a melanocortin receptor 4 agonist (e.g., setmelanotide), a melanin concentrating hormone receptor 1 antagonist, a farnesoid X receptor (FXR) agonist (e.g. obeticholic acid), zonisamide, phentermine (alone or in combination with topiramate), a norepinephrine/dopamine reuptake inhibitor (e.g., buproprion), an opioid receptor antagonist (e.g., naltrexone), a combination of norepinephrine/dopamine reuptake inhibitor and opioid receptor antagonist (e.g., a combination of bupropion and naltrexone), a GDF-15 analog, sibutramine, a cholecystokinin agonist, amylin and analogues therof (e.g., pramlintide), leptin and analogues thereof (e.g., metroleptin), a serotonergic agent (e.g., lorcaserin), a methionine aminopeptidase 2 (MetAP2) inhibitor (e.g., beloranib or ZGN-1061), phendimetrazine, diethylpropion, benzphetamine, an SGLT2 inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL1 inhibitor, a dual SGLT2/SGLT1 inhibitor, a fibroblast growth factor receptor (FGFR) modulator, an AMP-activated protein kinase (AMPK) activator, biotin, a MAS receptor modulator, or a glucagon receptor agonist (alone or in combination with another GLP-1 R agonist, e.g., liraglutide, exenatide, dulaglutide, albiglutide, lixisenatide, or semaglutide), including the pharmaceutically acceptable salts of the specifically named agents and the pharmaceutically acceptable solvates of said agents and salts.

In some embodiments, the one or more other therapeutic agent is an agent to treat NASH including but not limited to PF-05221304, an FXR agonist (e.g., obeticholic acid), a PPAR a/d agonist (e.g., elafibranor), a synthetic fatty acid-bile acid conjugate (e.g., aramchol), a caspase inhibitor (e.g., emricasan), an anti-lysyl oxidase homologue 2 (LOXL2) monoclonal antibody (e.g., simtuzumab), a galectin 3 inhibitor (e.g., GR-MD-02), a MAPK5 inhibitor (e.g., GS-4997), a dual antagonist of chemokine receptor 2 (CCR2) and CCR5 (e.g., cenicriviroc), a fibroblast growth factor21 (FGF21) agonist (e.g., BMS-986036), a leukotriene D4 (LTD4) receptor antagonist (e.g., tipelukast), a niacin analogue (e.g., ARI 3037MO), an ASBT inhibitor (e.g., volixibat), an acetyl-CoA carboxylase (ACC) inhibitor (e.g., NDI 010976), a ketohexokinase (KHK) inhibitor, a diacylglyceryl acyltransferase 2 (DGAT2) inhibitor, a CB1 receptor antagonist, an anti-CB1 R antibody, or an apoptosis signal-regulating kinase 1 (ASK1) inhibitor, including the pharmaceutically acceptable salts of the specifically named agents and the pharmaceutically acceptable solvates of said agents and salts.

### Articles of Manufacture and Kits

The present disclosure further provides articles of manufacture comprising a compound, or a stereoisomer, tautomer, or a pharmaceutically acceptable salt of any of the foregoing in accordance with the present application, a composition described herein, or one or more unit dosages described herein in suitable packaging. In certain embodiments, the article of manufacture is for use in any of the methods described herein. Suitable packaging (e.g., containers) is known in the art and includes, for example, vials, vessels, ampules, bottles, jars, flexible packaging and the like. An article of manufacture may further be sterilized and/or sealed.

The kits may be in unit dosage forms, bulk packages (e.g., multi-dose packages) or sub-unit doses. For example, kits may be provided that contain sufficient dosages of a compound, or a stereoisomer, tautomer, or a pharmaceutically acceptable salt of any of the foregoing in accordance with the present application, a composition described herein, and/or one or more other therapeutic agent useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds/compositions described herein and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### Enumerated Embodiments

1. A compound of formula (I):

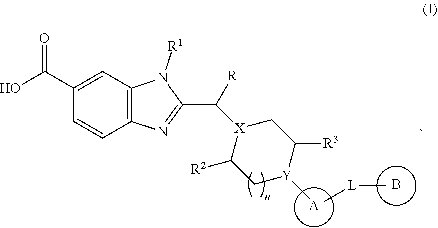

or a stereoisomer, tautomer, or a pharmaceutically acceptable salt of any of the foregoing, wherein:
X is N or CH;
Y is N or CR⁴, wherein R⁴ is hydrogen, OH or C₁-C₆ alkyl;
n is 0 or 1;
R is hydrogen;
R¹ is —C₁-C₆ alkylene-R⁵, wherein R⁵ is 3- to 6-membered heterocyclyl or 5- to 6-membered heteroaryl, each of which is independently optionally substituted by C₁-C₆ alkyl, or
   R¹ is taken together with R and the intervening atoms to form a Ring C, wherein Ring C is a 5- to 7-membered heterocyclyl optionally substituted by C₁-C₆ alkyl;
R² and R³ are independently hydrogen, oxo, or C₁-C₆ alkyl, wherein when Y is CR⁴, R³ and
R⁴ are optionally taken together with the carbon atoms to which they are attached to form C₃-C₆ cycloalkyl;
Ring A is 5- to 12-membered heterocyclyl or 5- to 12-membered heteroaryl, each of which is independently optionally substituted by halo, CN, C₃-C₆ cycloalkyl, or C₁-C₆ alkyl optionally substituted by halo or OH;
L is a bond, —O—, C₁-C₆ alkylene, *—O—C₁-C₆ alkylene-**, *—C₁-C₆ alkylene-O—**, or *—NR⁶—C₁-C₆ alkylene-**, wherein
   * represents the point of attachment to ring A and ** represents the point of attachment to ring B,
   when L is *—O—C₁-C₆ alkylene-**, the C₁-C₆ alkylene is optionally substituted by R^{L}, wherein:
   each R^{L} is independently C₁-C₆ alkyl, or
two R^{L} are taken together with the carbon atom or atoms to which they are attached to form C₃-C₆ cycloalkyl or 3- to 6-membered heterocyclyl, and
   R⁶ is hydrogen or C₁-C₆ alkyl; and
Ring B is C₃-C₁₀ cycloalkyl, C₆-C₁₄ aryl, 4- to 12-membered heterocyclyl, or 5- to 12-membered heteroaryl, each of which is independently optionally substituted by one to three substituents independently selected from the group consisting of halo, CN, oxo, C₁-C₆ alkyl, —COCH₃, —CONH₂, —S(O)₂CH₃, and phenyl,
with the proviso that
   when R¹ is —C₁-C₆ alkylene-R⁵, wherein R⁵ is 3- to 6-membered heterocyclyl or 3- to 6-membered heteroaryl, each of which is optionally substituted by C₁-C₆ alkyl, Y is N or CH, n is 1, R² and R³ are independently hydrogen or C₁-C₆ alkyl, ring A is 6-membered heteroaryl optionally substituted one or two substituents each independently selected from the group consisting of F, Cl and CN, and L is *—OCH₂—**, then ring B is not phenyl optionally substituted by one or two substituents each independently selected from the group consisting of halo, CN, and C₁-C₆ alkyl;
   when R¹ is —C₁-C₆ alkylene-R⁵, wherein R⁵ is 3- to 6-membered heterocyclyl or 3- to 6-membered heteroaryl, each of which is optionally substituted by C₁-C₆ alkyl, Y is N or CH, n is 1, R² and R³ are independently hydrogen or C₁-C₆ alkyl, ring A is

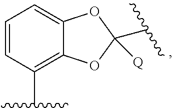

wherein Q is H or CH₃, and L is a bond, then ring B is neither phenyl or pyridinyl, each of which is optionally substituted by one or two substituents each independently selected from the group consisting of halo, CN, and C₁-C₆ alkyl; and
when R¹ is —C₁-C₆ alkylene-R⁵, wherein R⁵ is 4-membered heterocyclyl or 5-membered heteroaryl, each of which is optionally substituted by C₁-C₆ alkyl, X is N, Y is N or CH, n is 1, and R² and R³ are independently hydrogen or oxo, then ring B is not

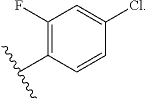 [SMILES: *c1ccc(Cl)cc1F]

2. The compound of embodiment 1, or a stereoisomer, tautomer, or a pharmaceutically acceptable salt of any of the foregoing, wherein R¹ is —C₁-C₆ alkylene-R⁵.
3. The compound of embodiment 1 or 2, or a stereoisomer, tautomer, or a pharmaceutically acceptable salt of any of the foregoing, wherein R¹ is —CH₂—R⁵.
4. The compound of any one of embodiments 1-3, or a stereoisomer, tautomer, or a pharmaceutically acceptable salt of any of the foregoing, wherein R⁵ is 3- to 6-membered heterocyclyl, which is optionally substituted by C₁-C₆ alkyl.
5. The compound of embodiment 4, or a stereoisomer, tautomer, or a pharmaceutically acceptable salt of any of the foregoing, wherein R⁵ is

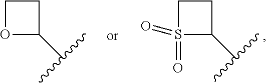 [SMILES: *C1CCO1 ; *C1CCS1(=O)=O]

each of which is independently optionally substituted by C₁-C₆ alkyl.
6. The compound of any one of embodiments 1-3, or a stereoisomer, tautomer, or a pharmaceutically acceptable salt of any of the foregoing, wherein R⁵ is 5- to 6-membered heteroaryl, which is optionally substituted by C₁-C₆ alkyl.
7. The compound of embodiment 6, or a stereoisomer, tautomer, or a pharmaceutically acceptable salt of any of the foregoing, wherein R⁵ is

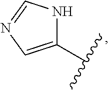 [SMILES: *c1cnc[nH]1]

which is optionally substituted by C₁-C₆ alkyl.
8. The compound of embodiment 1, or a stereoisomer, tautomer, or a pharmaceutically acceptable salt of any of the foregoing, wherein the compound is of formula (II),

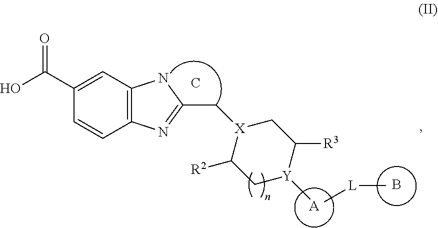

wherein Ring C is a 5- to 7-membered heterocyclyl optionally substituted by C₁-C₆ alkyl.
9. The compound of embodiment 1 or 8, or a stereoisomer, tautomer, or a pharmaceutically acceptable salt of any of the foregoing, wherein Ring C is

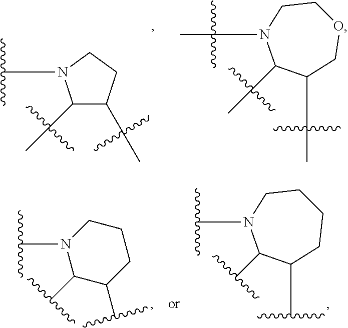 [SMILES: *C1CCN(*)C1* ; *C1COCCN(*)C1* ; *C1CCCN(*)C1* ; *C1CCCCN(*)C1*]

each of which is independently optionally substituted by C₁-C₆ alkyl.
10. The compound of embodiment 1, or a stereoisomer, tautomer, or a pharmaceutically acceptable salt of any of the foregoing, wherein the compound is of formula (III),

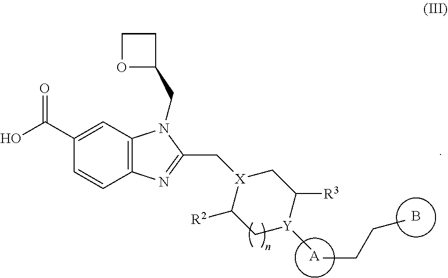

11. The compound of embodiment 1, or a stereoisomer, tautomer, or a pharmaceutically acceptable salt of any of the foregoing, wherein the compound is of formula (IV),

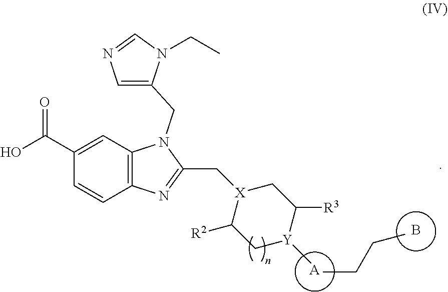

12. The compound of any one of embodiments 1-11, or a stereoisomer, tautomer, or a pharmaceutically acceptable salt of any of the foregoing, wherein X is N.
13. The compound of any one of embodiments 1-11, or a stereoisomer, tautomer, or a pharmaceutically acceptable salt of any of the foregoing, wherein X is CH.
14. The compound of any one of embodiments 1-13, or a stereoisomer, tautomer, or a pharmaceutically acceptable salt of any of the foregoing, wherein n is 0.
15. The compound of any one of embodiments 1-13, or a stereoisomer, tautomer, or a pharmaceutically acceptable salt of any of the foregoing, wherein n is 1.
16. The compound of any one of embodiments 1-15, or a stereoisomer, tautomer, or a pharmaceutically acceptable salt of any of the foregoing, wherein Y is N.
17. The compound of any one of embodiments 1-15, or a stereoisomer, tautomer, or a pharmaceutically acceptable salt of any of the foregoing, wherein Y is CR⁴.
18. The compound of embodiment 17, or a stereoisomer, tautomer, or a pharmaceutically acceptable salt of any of the foregoing, wherein R³ and R⁴ are taken together with the carbon atoms to which they are attached to form a C₃-C₆ cycloalkyl.
19. The compound of embodiment 18, or a stereoisomer, tautomer, or a pharmaceutically acceptable salt of any of the foregoing, wherein R³ and R⁴ are taken together with the carbon atoms to which they are attached to form cyclopropyl.
20. The compound of any of one of embodiments 1-19, or a stereoisomer, tautomer, or a pharmaceutically acceptable salt of any of the foregoing, wherein Ring A is 5- to 12-membered heterocyclyl, which is optionally substituted by halo, CN, C₃-C₆ cycloalkyl, or C₁-C₆ alkyl optionally substituted by halo or OH.
21. The compound of embodiment 20, or a stereoisomer, tautomer, or a pharmaceutically acceptable salt of any of the foregoing, wherein Ring A is

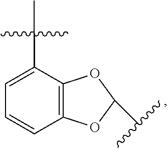 [SMILES: *c1cccc2c1OC(*)O2]

which is optionally substituted by halo, CN, C₃-C₆ cycloalkyl, or C₁-C₆ alkyl optionally substituted by halo or OH.
22. The compound of any of one of embodiments 1-19, or a stereoisomer, tautomer, or a pharmaceutically acceptable salt of any of the foregoing, wherein Ring A is 5- to 12-membered heteroaryl, which is optionally substituted by halo, CN, C₃-C₆ cycloalkyl, or C₁-C₆ alkyl optionally substituted by halo or OH.
23. The compound of embodiment 22, or a stereoisomer, tautomer, or a pharmaceutically acceptable salt of any of the foregoing, Ring A is

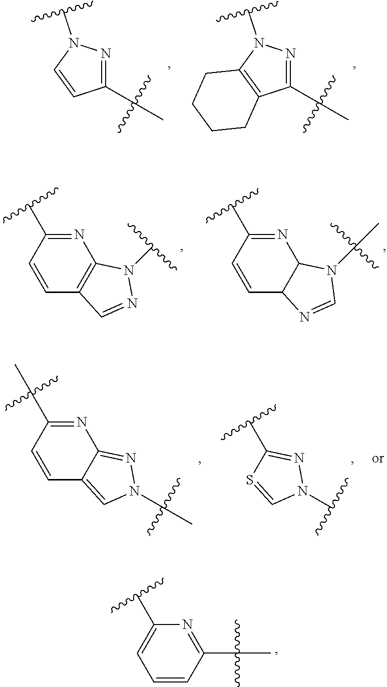

each of which is independently optionally substituted by halo, CN, C₃-C₆ cycloalkyl, or C₁-C₆ alkyl optionally substituted by halo or OH.
24. The compound of any one of embodiments 1-9 and 12-23, or a stereoisomer, tautomer, or a pharmaceutically acceptable salt of any of the foregoing, wherein L is a bond.
25. The compound of any one of embodiments 1-9 and 12-23, or a stereoisomer, tautomer, or a pharmaceutically acceptable salt of any of the foregoing, wherein L is —O—.
26. The compound of any one of embodiments 1-9 and 12-23, or a stereoisomer, tautomer, or a pharmaceutically acceptable salt of any of the foregoing, wherein L is C₁-C₆ alkylene.
27. The compound of any one of embodiments 1-9 and 12-23, or a stereoisomer, tautomer, or a pharmaceutically acceptable salt of any of the foregoing, wherein L is *—O—C₁-C₆ alkylene-**.
28. The compound of any one of embodiments 1-9 and 12-23, or a stereoisomer, tautomer, or a pharmaceutically acceptable salt of any of the foregoing, wherein L is *—C₁-C₆ alkylene-O—**.
29. The compound of any one of embodiments 1-9 and 12-23, or a stereoisomer, tautomer, or a pharmaceutically acceptable salt of any of the foregoing, wherein L is *—NR⁶—C₁-C₆ alkylene-**.
30. The compound of any one of embodiments 1-29, or a stereoisomer, tautomer, or a pharmaceutically acceptable salt of any of the foregoing, wherein Ring B is C₃-C₁₀ cycloalkyl, which is optionally substituted by one to three substituents independently selected from the group consisting of halo, CN, oxo, C₁-C₆ alkyl, —COCH₃, —CONH₂, —S(O)₂CH₃ and phenyl.
31. The compound of embodiment 30, or a stereoisomer, tautomer, or a pharmaceutically acceptable salt of any of the foregoing, wherein Ring B is

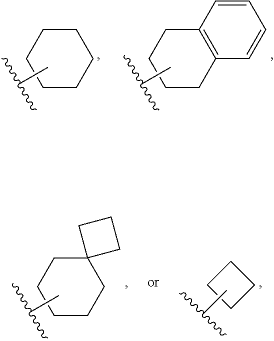

each of which is independently optionally substituted by one to three substituents independently selected from the group consisting of halo, CN, oxo, C₁-C₆ alkyl, —COCH₃, —CONH₂, —S(O)₂CH₃ and phenyl.
32. The compound of any one of embodiments 1-29, or a stereoisomer, tautomer, or a pharmaceutically acceptable salt of any of the foregoing, wherein Ring B is C₆-C₁₄ aryl, which is optionally substituted by one to three substituents independently selected from the group consisting of halo, CN, oxo, C₁-C₆ alkyl, —COCH₃, —CONH₂, —S(O)₂CH₃ and phenyl.
33. The compound of embodiment 32, or a stereoisomer, tautomer, or a pharmaceutically acceptable salt of any of the foregoing, wherein Ring B is

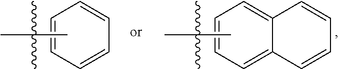

each of which is independently optionally substituted by one to three substituents each independently selected from the group consisting of halo, CN, oxo, C₁-C₆ alkyl, —COCH₃, —CONH₂, —S(O)₂CH₃ and phenyl.
34. The compound of any one of embodiments 1-29, or a stereoisomer, tautomer, or a pharmaceutically acceptable salt of any of the foregoing, wherein Ring B is 4- to 12-membered heterocyclyl, which is optionally substituted by one to three substituents independently selected from the group consisting of halo, CN, oxo, C₁-C₆ alkyl, —COCH₃, —CONH₂, —S(O)₂CH₃ and phenyl.
35. The compound of embodiment 34, or a stereoisomer, tautomer, or a pharmaceutically acceptable salt of any of the foregoing, wherein Ring B is

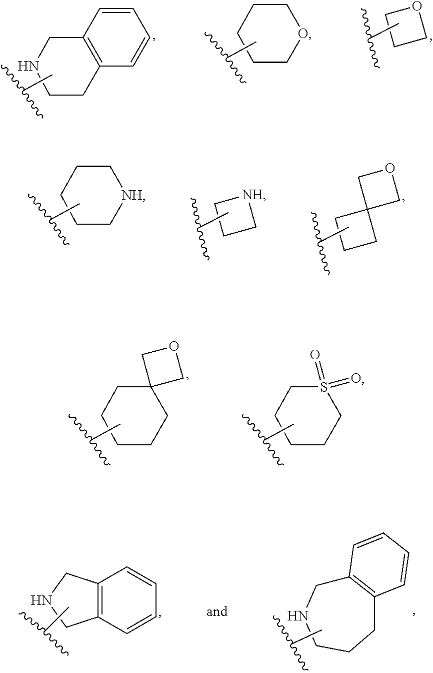

each of which is independently optionally substituted by one to three substituents independently selected from the group consisting of halo, CN, oxo, C₁-C₆ alkyl, —COCH₃, —CONH₂, —S(O)₂CH₃ and phenyl.
36. The compound of any one of embodiments 1-29, or a stereoisomer, tautomer, or a pharmaceutically acceptable salt of any of the foregoing, wherein Ring B is 5- to 12-membered heteroaryl, which is optionally substituted by one to three substituents independently selected from the group consisting of halo, CN, oxo, C₁-C₆ alkyl, —COCH₃, —CONH₂, —S(O)₂CH₃ and phenyl.
37. The compound of embodiment 36, or a stereoisomer, tautomer, or a pharmaceutically acceptable salt of any of the foregoing, wherein Ring B is N

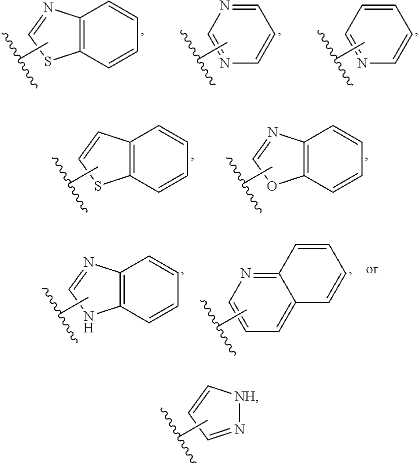

each of which is independently optionally substituted by one to three substituents independently selected from the group consisting of halo, CN, oxo, C₁-C₆ alkyl, —COCH₃, —CONH₂, —S(O)₂CH₃ and phenyl.
38. A compound or a pharmaceutically acceptable salt thereof, wherein the compound is selected from the group consisting of the compounds in Table 1.
39. A pharmaceutical composition comprising the compound of any one of embodiments 1-38, or a stereoisomer, tautomer, or a pharmaceutically acceptable salt of any of the foregoing, and a pharmaceutical acceptable excipient.
40. A method of treating a disease mediated by glucagon-like peptide-1 receptor (GLP-1R) in an individual in need thereof, comprising administering to the individual a therapeutically effective amount of the compound of any one of embodiments 1-38, or a stereoisomer, tautomer, or a pharmaceutically acceptable salt of any of the foregoing, or the pharmaceutical composition of embodiment 39.
41. The method of embodiment 40, wherein the disease is a liver disease.
42. The method of embodiment 41, wherein the liver disease is primary biliary cirrhosis (PBC), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, nonalcoholic fatty liver disease (NAFLD), nonalcoholic steatohepatitis (NASH), graft versus host disease, transplant liver regeneration, congenital hepatic fibrosis, choledocholithiasis, granulomatous liver disease, intra- or extrahepatic malignancy, Sjogren's syndrome, sarcoidosis, Wilson's disease, Gaucher's disease, hemochromatosis, or oti-antitrypsin deficiency.
43. The method of embodiment 40, wherein the disease is diabetes.
44. The method of embodiment 40, wherein the disease is a cardiometabolic disease.
45. Use of the compound of any one of embodiments 1-38, or a stereoisomer, tautomer, or a pharmaceutically acceptable salt of any of the foregoing, in the manufacture of a medicament for treating a disease mediated by mediated by GLP-1R.

### EXAMPLES

### Part I: General Procedures

### Preparing Compounds: Synthetic Schemes

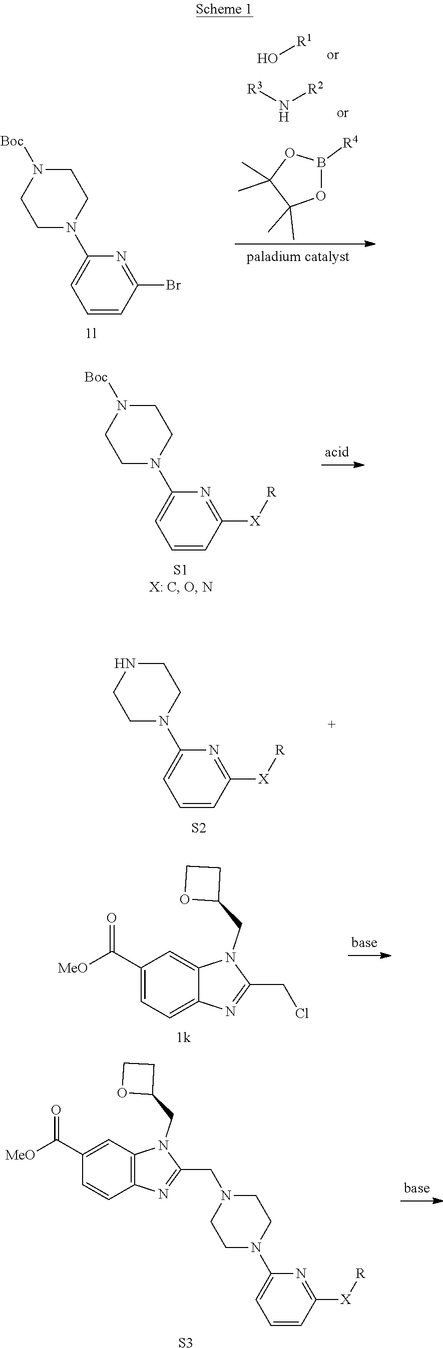

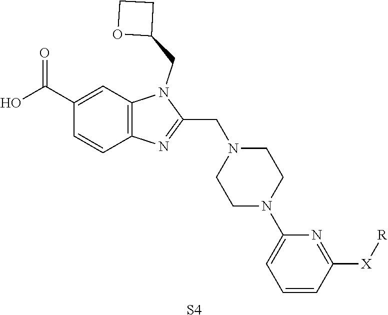

Compounds of formula (S4) may be prepared by general synthetic methods as shown in Scheme 1. Compounds of formula (S1) can be prepared from the 2-bromopyridine (11) upon treatment with Alkyl or aryl alcohols, Alkyl or aryl amine and aryl boronic acids or boronate esters under palladium catalyst conditions such as, but not limited to, XantPhos Pd G4 and an inorganic base such as, but not limited to, cesium carbonate in an organic solvent such as, but not limited to, toluene at an elevated temperature. Treatment of the N-Boc (51) with acid such as, but not limited to, trifluoroacetic acid and organic solvents such as, but not limited to, dichloromethane yields formula (S2). Compounds of formula (S3) can be prepared from the benzyl chloride (1k) upon treatment with amine (S2) under base such as, but not limited to, potassium carbonate. Treatment of the ester (S3) with hydroxide sources such as, but not limited to, lithium hydroxide in the presence of water and organic solvents such as, but not limited to, methanol and/or tetrahydrofuran yields carboxylic acid of formula (S4).

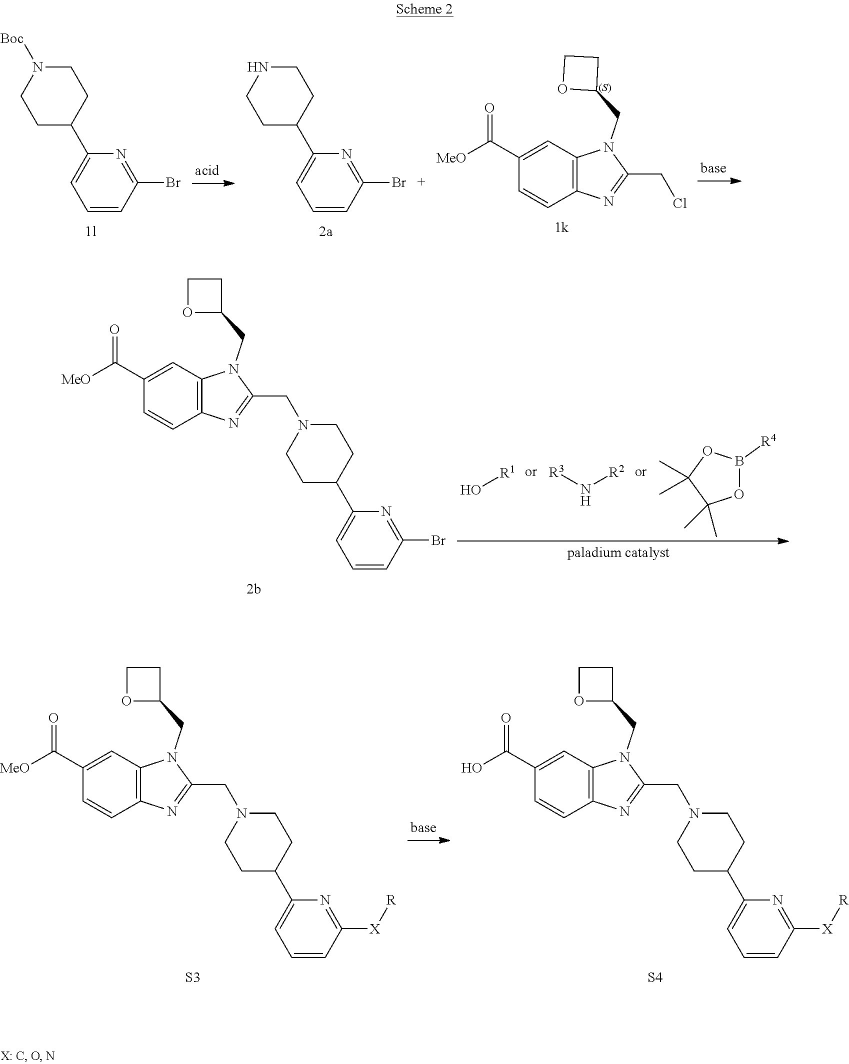

Compounds of formula (S4) may be prepared by general synthetic methods as shown in Scheme 2. Treatment of the N-Boc (11) with acid such as, but not limited to, trifluoroacetic acid and organic solvents such as, but not limited to, dichloromethane yields formula (2a). Compounds of formula (2b) can be prepared from the benzyl chloride (1k) upon treatment with amine (2a) under base such as, but not limited to, potassium carbonate. Compounds of formula (S3) can be prepared from the 2-bromopyridine (2b) upon treatment with Alkyl or aryl alcohols, Alkyl or aryl amine and aryl boronic acids or boronate esters under palladium catalyst conditions such as, but not limited to, XantPhos Pd G4 and an inorganic base such as, but not limited to, cesium carbonate in an organic solvent such as, but not limited to, toluene at an elevated temperature. Treatment of the ester (S3) with hydroxide sources such as, but not limited to, lithium hydroxide in the presence of water and organic solvents such as, but not limited to, methanol and/or tetrahydrofuran yields carboxylic acid of formula (S4).

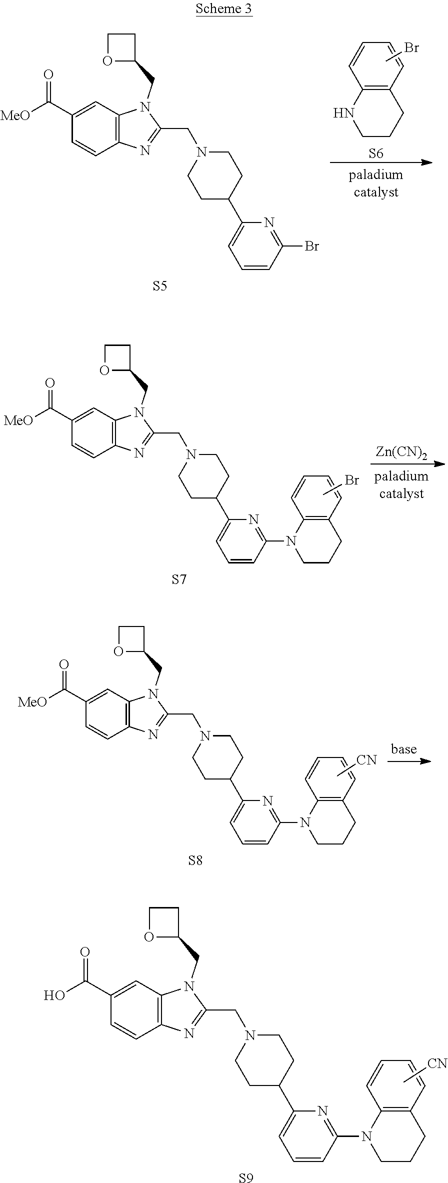

Compounds of formula (S9) may be prepared by general synthetic methods as shown in Scheme 3. Compounds of formula (S7) can be prepared from the 2-bromopyridine (S5) upon treatment with aryl amine under palladium catalyst conditions such as, but not limited to, XantPhos Pd G4 and an inorganic base such as, but not limited to, cesium carbonate in an organic solvent such as, but not limited to, toluene at an elevated temperature. Compounds of formula (S8) can be prepared from the bromobenzene (S7) upon treatment with zinc cyanide under palladium catalyst conditions such as, but not limited to, Pd(PPh3)4 and an organic solvent such as, but not limited to, toluene at an elevated temperature. Treatment of the ester (S8) with hydroxide sources such as, but not limited to, lithium hydroxide in the presence of water and organic solvents such as, but not limited to, methanol and/or tetrahydrofuran yields carboxylic acid of formula (S9).

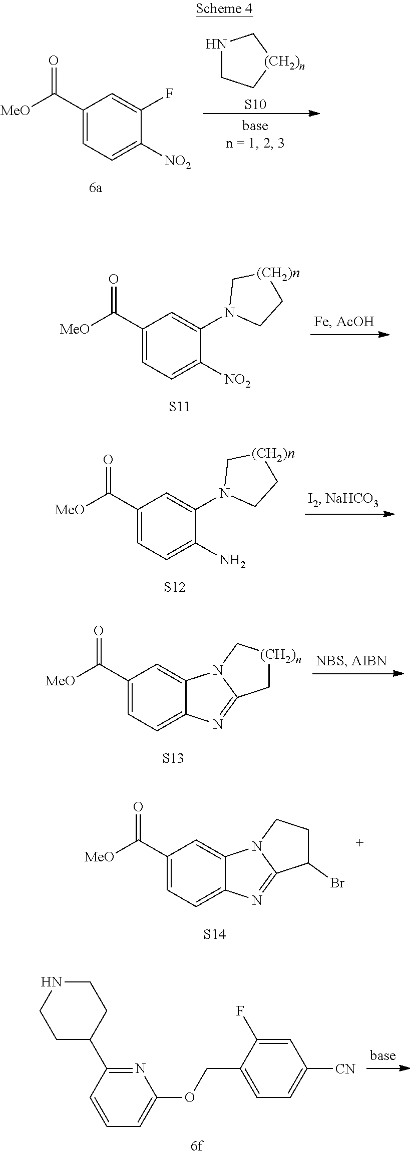

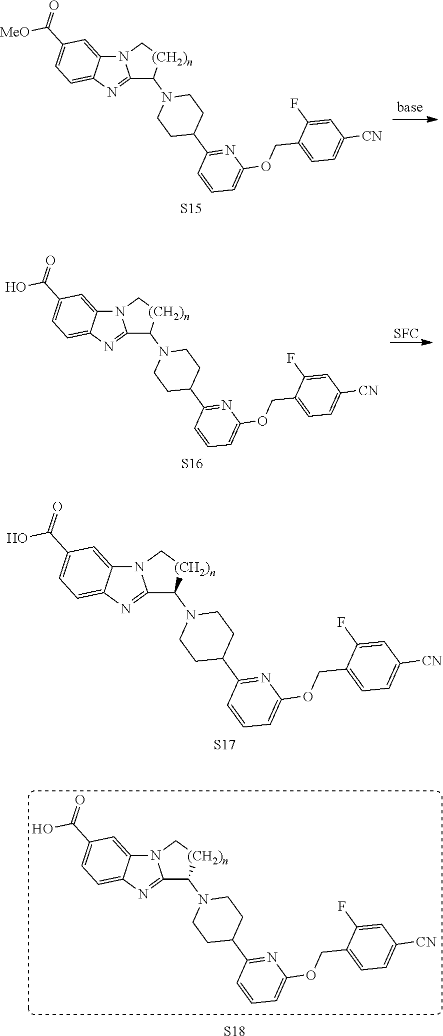

Compounds of formula (S17& S18) may be prepared by general synthetic methods as shown in Scheme 4. Treatment of cyclamine (S10) with fluorobenzene (6a) in a suitable solvent such as ethanol with a base such as, but not limited to, triethylamine at a temperature from about room temperature to 35° C. and for a time varying from about 3 hours to about 16 hours, can readily produce nitroaniline (S11). The phenylenediamine (S12) can be formed by reduction of nitroaniline (S11) using a reductant such as, but not limited to, iron in a solvent such as, but not limited to acetic acid at a temperature from about room temperature to 40° C. and for a time varying from about 1 hour. The cyclization of phenylenediamine (S12) to compounds of formula (S13) can be carried out using a reagent such as, but not limited to, inodine and sodium bicarbonate in a suitable solvent such as ethanol, at a temperature from about room temperature and for a time varying from about 3 hours to about 16 hours. Bromine compound (S14) can be prepared from compounds of formula (S13) upon treatment with NBS under initiator conditions such as, but not limited to, AIBN in an organic solvent such as, but not limited to, carbon tetrachloride at a reflux temperature and for a time varying from about 3 hours to about 8 hours. Treatment of Bromine compound (S14) with secondary amine (6f) in a suitable solvent such as acetontrile with a base such as, but not limited to, potassium carbonate in the presence of activator such as, but not limited to, potassium iodide at a refluxed temperature and for a time varying from about 3 hours to about 8 hours, can readily produce ester (S15). Treatment of the ester (S15) with hydroxide sources such as, but not limited to, lithium hydroxide in the presence of water and organic solvents such as, but not limited to, methanol and/or tetrahydrofuran yields carboxylic acid of formula (S16). Carboxylic acid (S16) and Carboxylic acid (S16) was separated by SFC.

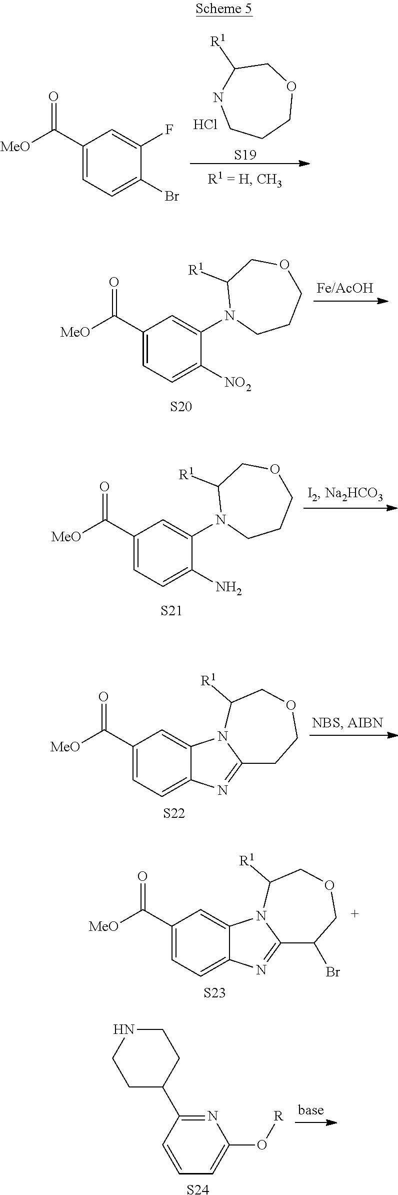

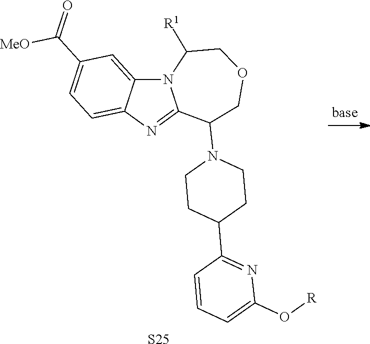

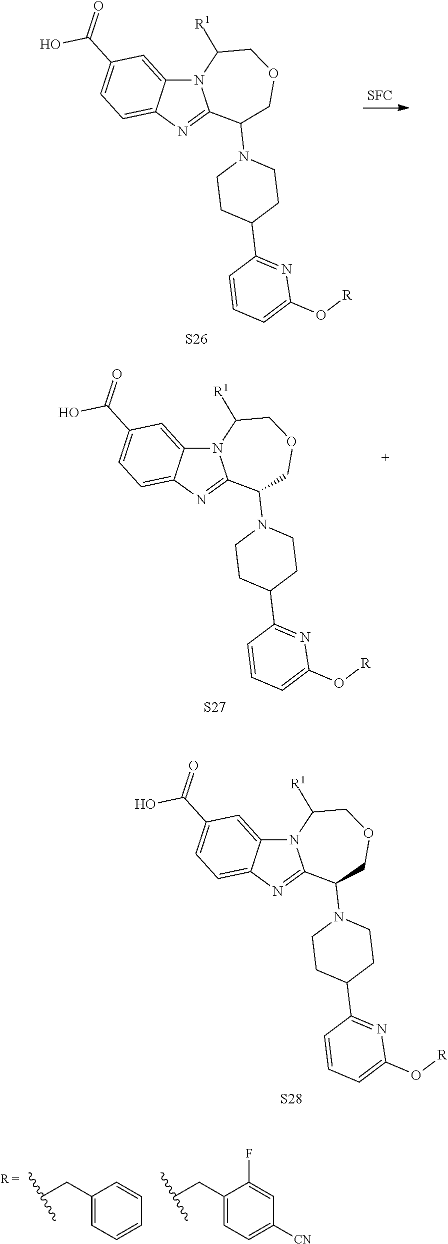

Compounds of formula (S27& S28) may be prepared by general synthetic methods as shown in Scheme 5. Treatment of epoxy amine compound (S19) with fluorobenzene (6a) in a suitable solvent such as ethanol with a base such as, but not limited to, triethylamine at a temperature from about room temperature to 35° C. and for a time varying from about 3 hours to about 16 hours, can readily produce nitroaniline (S20). The compounds of formula (S21) can be formed by reduction of nitroaniline (S20) using a reductant such as, but not limited to, iron in a solvent such as, but not limited to acetic acid at a temperature from about room temperature to 40° C. and for a time varying from about 1 hour. The cyclization of phenylenediamine (S22) to compounds of formula (S21) can be carried out using a reagent such as, but not limited to, inodine and sodium bicarbonate in a suitable solvent such as ethanol, at a temperature from about room temperature and for a time varying from about 3 hours to about 16 hours. Bromine compound (S23) can be prepared from phenylenediamine (S22) upon treatment with NBS under initiator conditions such as, but not limited to, AIBN in an organic solvent such as, but not limited to, carbon tetrachloride at a reflux temperature and for a time varying from about 3 hours to about 8 hours. Treatment of Bromine compound (S23) with secondary amine (S24) in a suitable solvent such as acetontrile with a base such as, but not limited to, potassium carbonate in the presence of activator such as, but not limited to, potassium iodide at a refluxed temperature and for a time varying from about 3 hours to about 8 hours, can readily produce ester (S25). Treatment of the ester (S25) with hydroxide sources such as, but not limited to, lithium hydroxide in the presence of water and organic solvents such as, but not limited to, methanol and/or tetrahydrofuran yields carboxylic acid of formula (S26). Carboxylic acid (S27) and Carboxylic acid (S28) was separated by SFC.

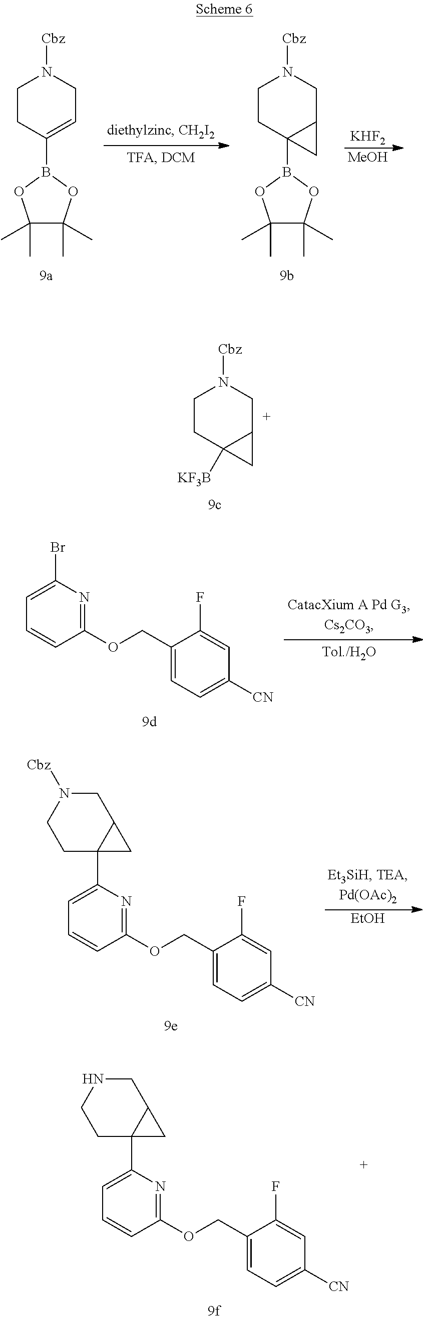

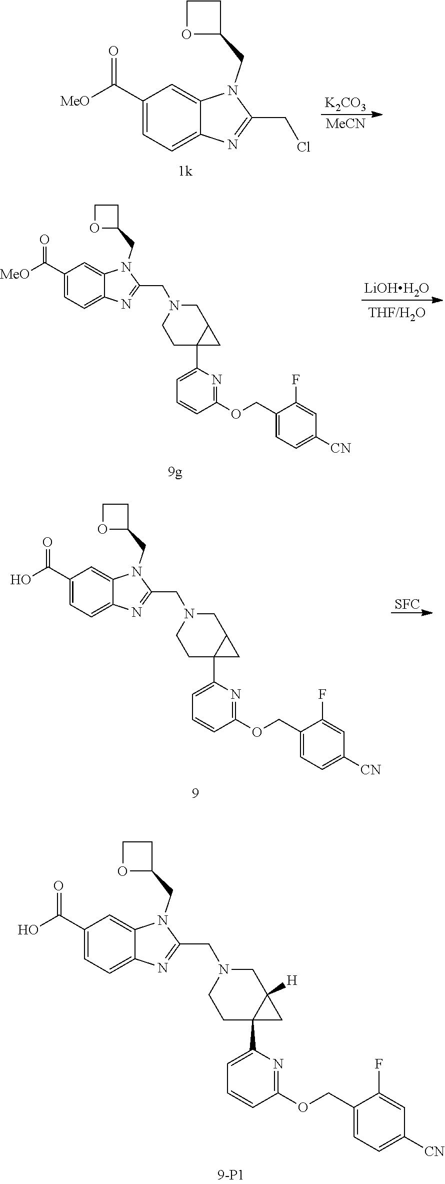

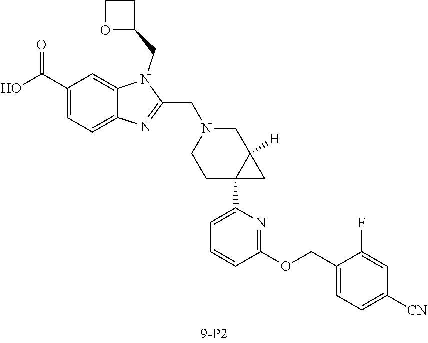 [SMILES: N#Cc1ccc(COc2cccc([C@]34CCN(Cc5nc6ccc(C(=O)O)cc6n5C[C@@H]5CCO5)C[C@H]3C4)n2)c(F)c1]

Scheme 6 can be used for the synthesis of Compound 9. Detailed procedures are described in Example 9.

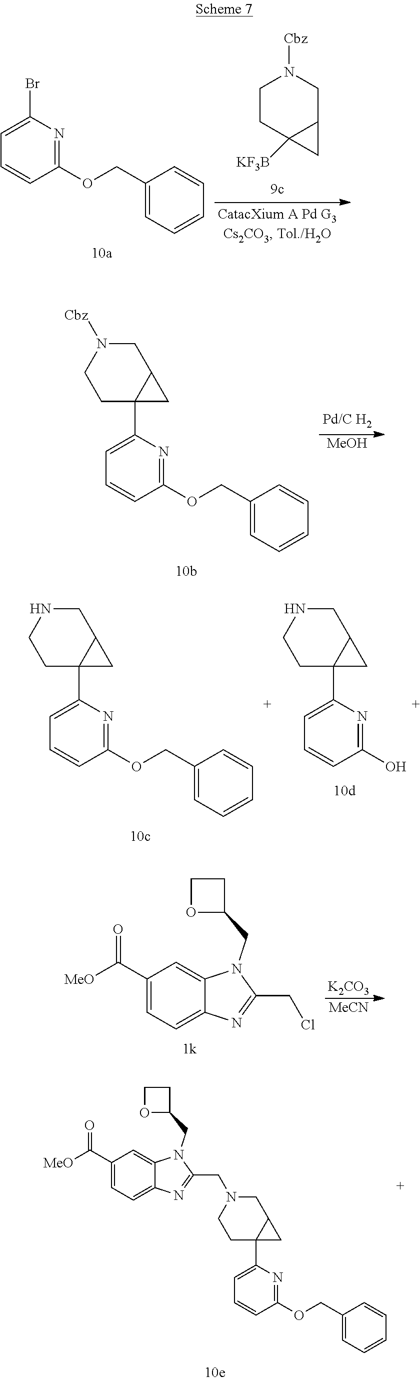

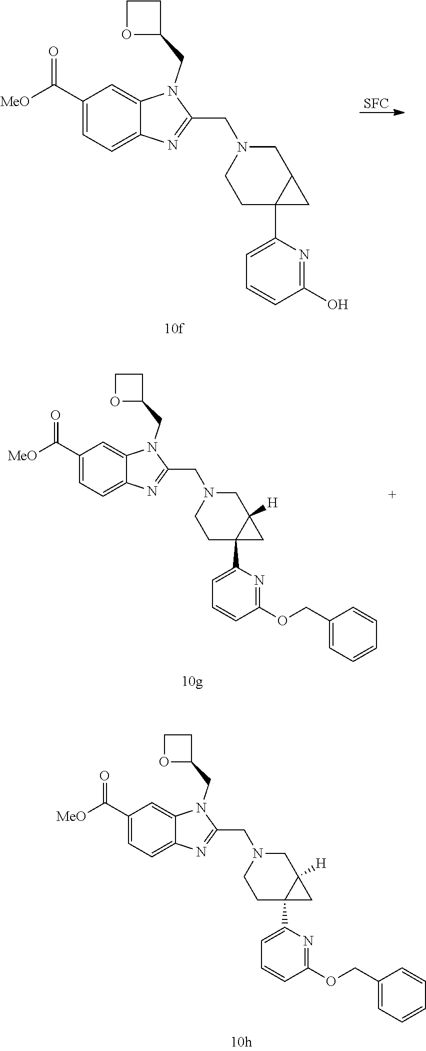 [SMILES: COC(=O)c1ccc2nc(CN3CCC4(c5cccc(O)n5)CC4C3)n(C[C@@H]3CCO3)c2c1 ; "SFC" ; COC(=O)c1ccc2nc(CN3CC[C@@]4(c5cccc(OCc6ccccc6)n5)C[C@H]4C3)n(C[C@@H]3CCO3)c2c1 ; COC(=O)c1ccc2nc(CN3CC[C@]4(c5cccc(OCc6ccccc6)n5)C[C@@H]4C3)n(C[C@@H]3CCO3)c2c1]

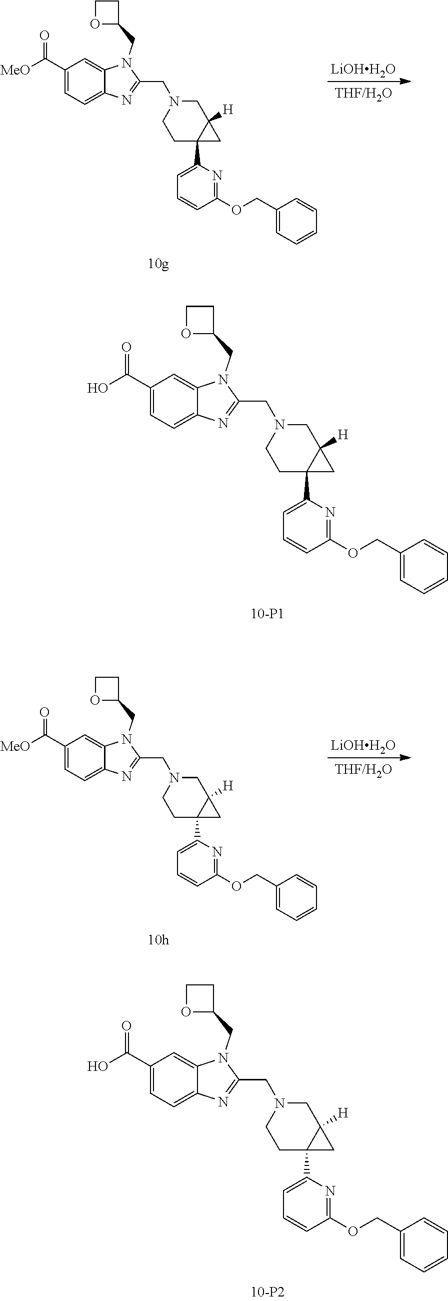 [SMILES: COC(=O)c1ccc2nc(CN3CC[C@@]4(c5cccc(OCc6ccccc6)n5)C[C@H]4C3)n(C[C@@H]3CCO3)c2c1 ; "LiOH·H2O" ; O=C(O)c1ccc2nc(CN3CC[C@@]4(c5cccc(OCc6ccccc6)n5)C[C@H]4C3)n(C[C@@H]3CCO3)c2c1 ; COC(=O)c1ccc2nc(CN3CC[C@]4(c5cccc(OCc6ccccc6)n5)C[C@@H]4C3)n(C[C@@H]3CCO3)c2c1 ; "LiOH·H2O" ; O=C(O)c1ccc2nc(CN3CC[C@]4(c5cccc(OCc6ccccc6)n5)C[C@@H]4C3)n(C[C@@H]3CCO3)c2c1]

Scheme 7 can be used for the synthesis of Compound 10. Detailed procedures are described in Example 10.

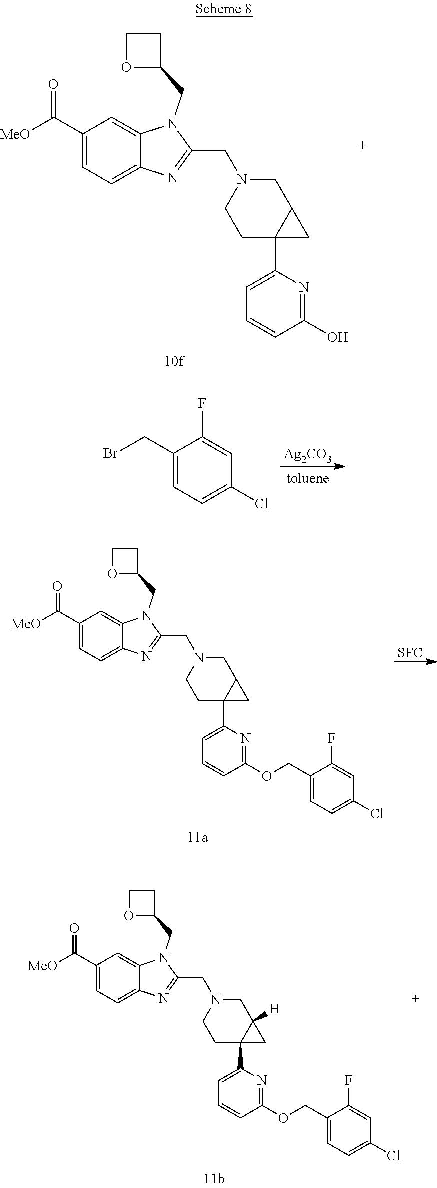

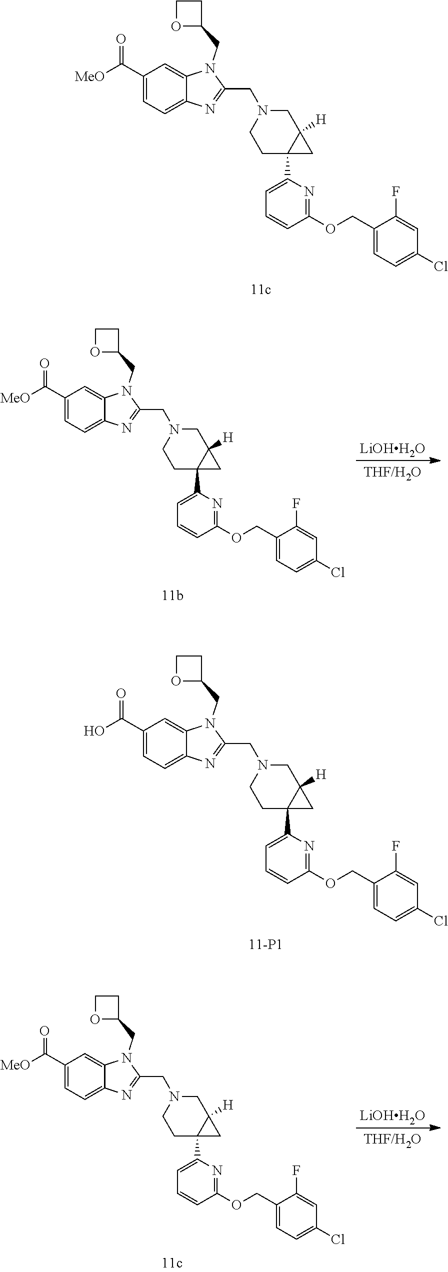 [SMILES: COC(=O)c1ccc2nc(CN3CC[C@]4(c5cccc(OCc6ccc(Cl)cc6F)n5)C[C@@H]4C3)n(C[C@@H]3CCO3)c2c1 ; COC(=O)c1ccc2nc(CN3CC[C@@]4(c5cccc(OCc6ccc(Cl)cc6F)n5)C[C@H]4C3)n(C[C@@H]3CCO3)c2c1 ; "LiOH·H2O" ; O=C(O)c1ccc2nc(CN3CC[C@@]4(c5cccc(OCc6ccc(Cl)cc6F)n5)C[C@H]4C3)n(C[C@@H]3CCO3)c2c1 ; COC(=O)c1ccc2nc(CN3CC[C@]4(c5cccc(OCc6ccc(Cl)cc6F)n5)C[C@@H]4C3)n(C[C@@H]3CCO3)c2c1 ; "LiOH·H2O"]

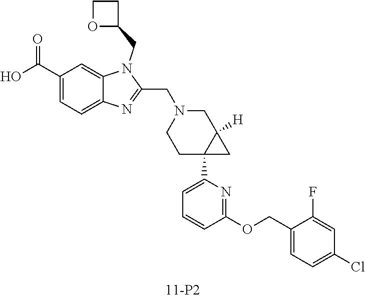 [SMILES: O=C(O)c1ccc2nc(CN3CC[C@]4(c5cccc(OCc6ccc(Cl)cc6F)n5)C[C@@H]4C3)n(C[C@@H]3CCO3)c2c1]

Scheme 8 can be used for the synthesis of Compound 11. Detailed procedures are described in Example 11.

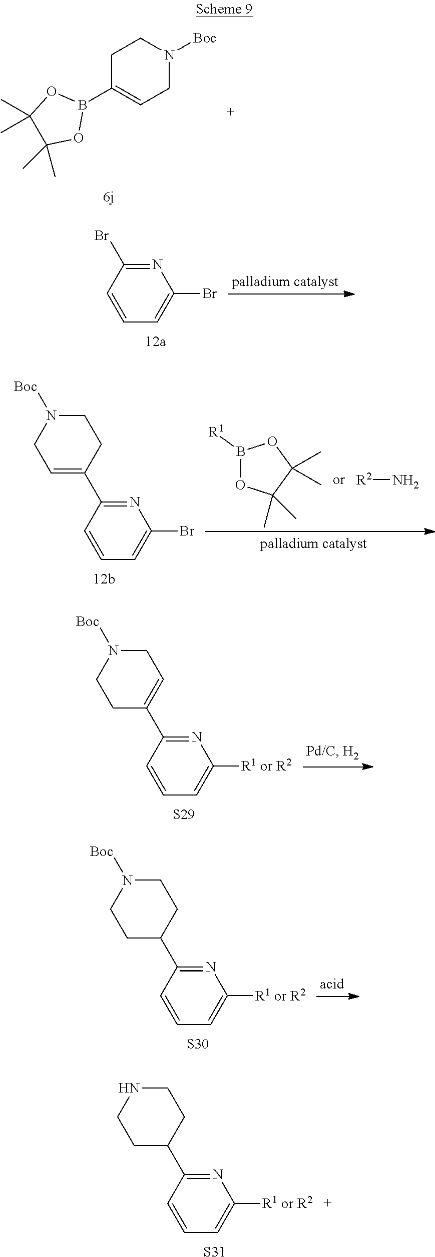

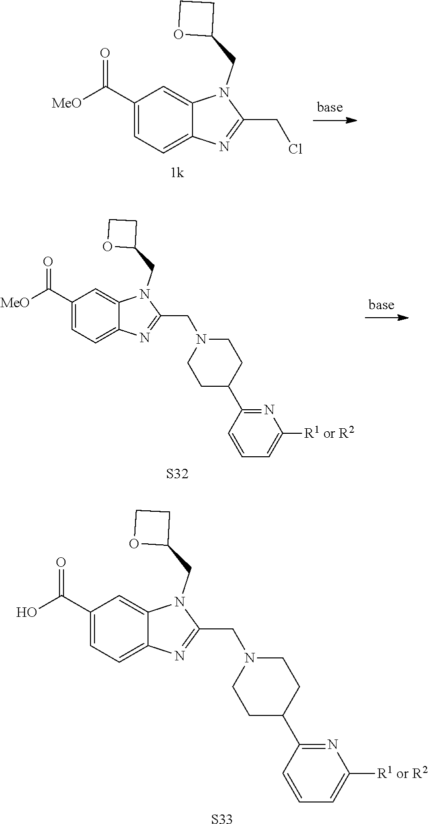

Compounds of formula (S33) may be prepared by general synthetic methods as shown in Scheme 9. Compounds of formula (12b) can be prepared from the 2,6-dibromopyridine (12a) upon treatment with boronate esters (6j) under palladium catalyst conditions such as, but not limited to, Pd(dppf)Cl₂·CH2Cl2 in the presence of water and an inorganic base such as, but not limited to, potassium carbonate in an organic solvent such as, but not limited to, DMSO at an elevated temperature for a time varying from about 16 hours under N2 atmosphere. Compounds of formula (S29) can be prepared from the bromopyridine (12b) upon treatment with boronate esters or amines under palladium catalyst conditions such as, but not limited to, Pd2(dba)3 in the presence of BINAP and an inorganic base such as, but not limited to, caesium carbonate in an organic solvent such as, but not limited to, toluene at an elevated temperature. In the presence Pd/C and H2, tert-butyl carbamate (S30) can be formed by reduction of compound of formula (S29) in a solvent such as, but not limited to methanol at room temperature and for a time varying from about 2 hours. Treatment of the tert-butyl carbamate (S30) with acid such as, but not limited to, THF in the presence of organic solvents such as, but not limited to, DCM yields amine of formula (S31). Compounds of formula (S32) can be prepared from the benzyl chloride (1k) upon treatment with amine (S31) under base such as, but not limited to, potassium carbonate. Treatment of the ester (S32) with hydroxide sources such as, but not limited to, lithium hydroxide in the presence of water and organic solvents such as, but not limited to, methanol and/or tetrahydrofuran yields carboxylic acid of formula (S33).

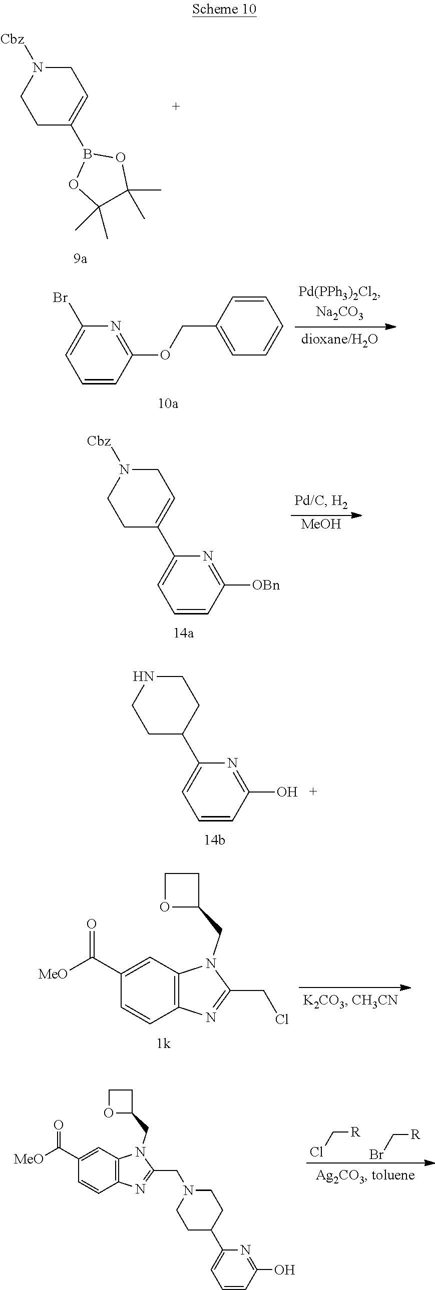

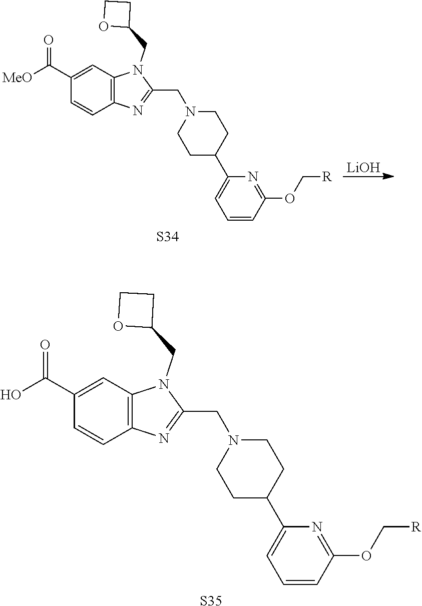

Compounds of formula (S35) may be prepared by general synthetic methods as shown in Scheme 10. Compounds of formula (14a) can be prepared from the bromopyridine (10a) upon treatment with boronate esters (9a) under palladium catalyst conditions such as, but not limited to, Pd(PPh3)2Cl2 in the presence of water and an inorganic base such as, but not limited to, sodium carbonate in an organic solvent such as, but not limited to, dioxane at an elevated temperature for a time varying from about 16 hours under N2 atmosphere. In the presence Pd/C and H2, amine (14b) can be formed by reduction of phenylmethanol (14a) in a solvent such as, but not limited to methanol at room temperature and for a time varying from about 6 hours. Compounds of formula (14c) can be prepared from the benzyl chloride (1k) upon treatment with amine (14b) under base such as, but not limited to, potassium carbonate. Treatment of benzimidazole (14c) with benzyl chloride or benzyl bromine in the presence of a base such as, but not limited to, Ag2CO3 in an organic solvent such as, but not limited to, toluene at a temperature at about 100° C. and for a time for about 16 hours to produce ester (S34). Treatment of the ester (S34) with hydroxide sources such as, but not limited to, lithium hydroxide in the presence of water and organic solvents such as, but not limited to, methanol and/or tetrahydrofuran yields carboxylic acid of formula (S35).

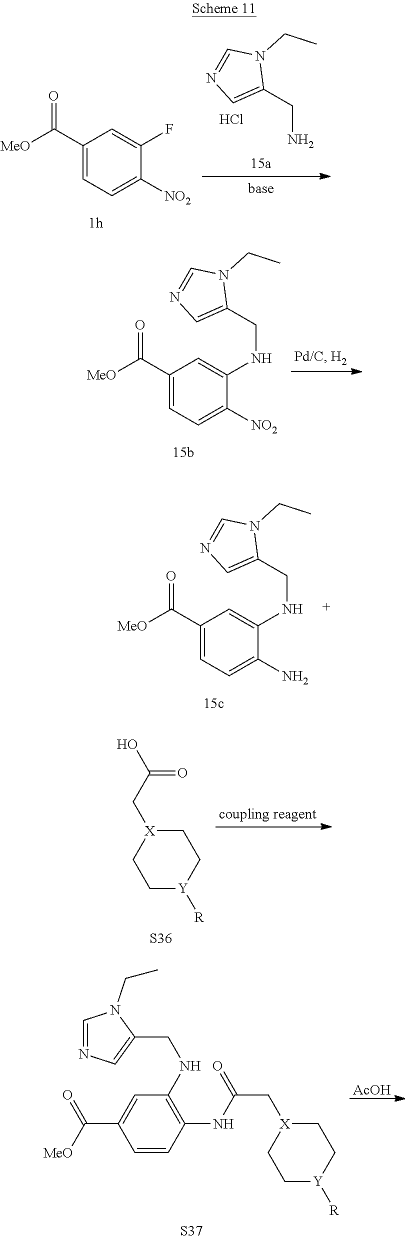

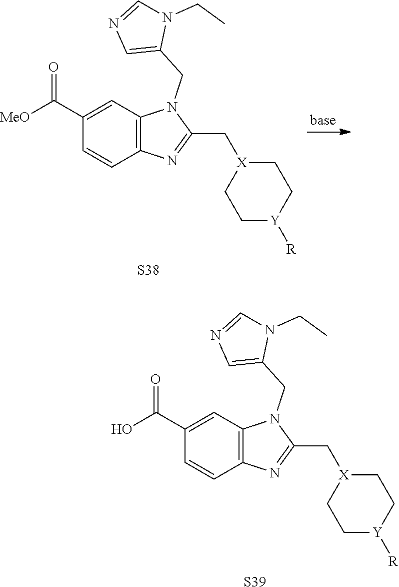

Compounds of formula (S39) may be prepared by general synthetic methods as shown in Scheme 11. Compounds of formula (15b) can be prepared from the fluorobenzene (1h) upon treatment with amine (15a) in the presence of water and an organic solvent such as, but not limited to, tetrahydrofuran under an inorganic base such as, but not limited to, triethylamine at an elevated temperature. In the presence Pd/C and H2, amine (15c) can be formed by reduction of nitrobenzene (15b) in a solvent such as, but not limited to methanol at room temperature and for a time varying from about 2 hours. Reaction of carboxylic acid (S36) with a coupling reagent such as, but not limited to, HATU, a base such as, but not limited to, diisopropylethylamine, and amine (15c) provides amide of formula (S37). Compound (S37) can be treated with AcOH at a temperature at about 65° C. and for a time of about 16 hours can afford compounds of formula (S38). Treatment of the ester (S38) with hydroxide sources such as, but not limited to, lithium hydroxide in the presence of water and organic solvents such as, but not limited to, methanol and/or tetrahydrofuran yields carboxylic acid of formula (S39).

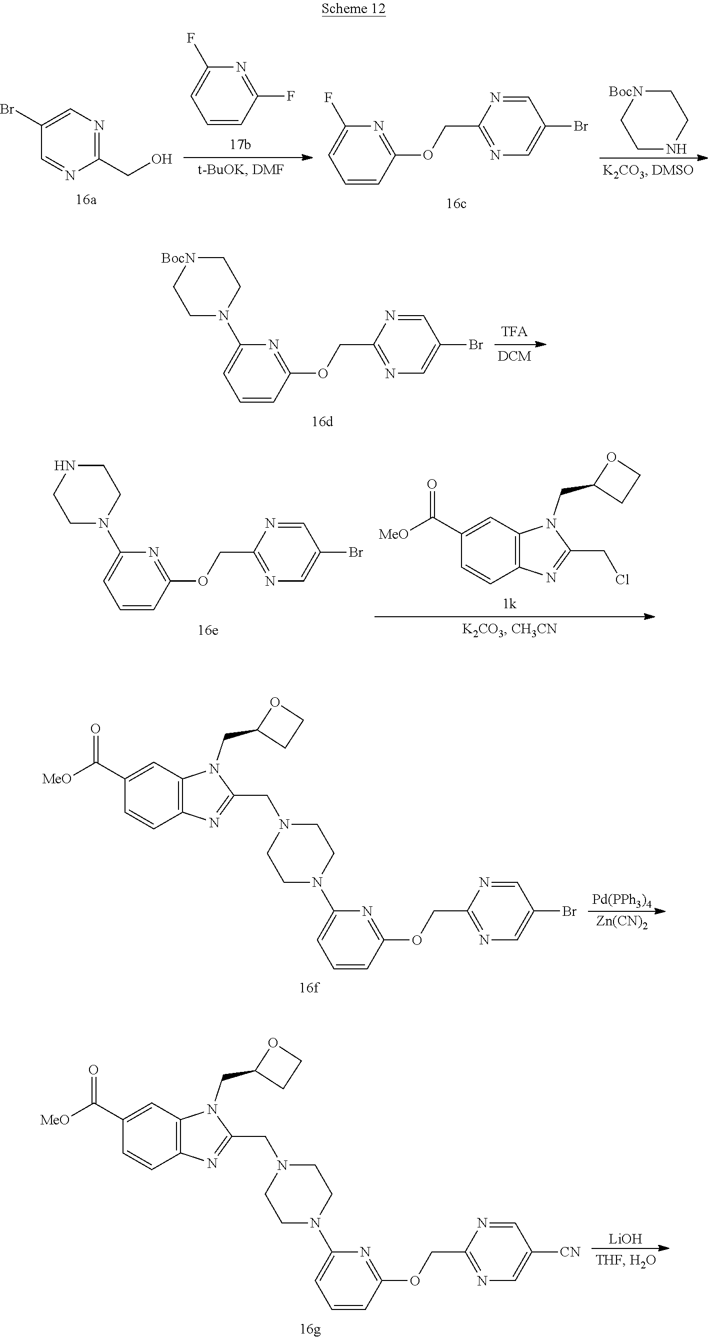

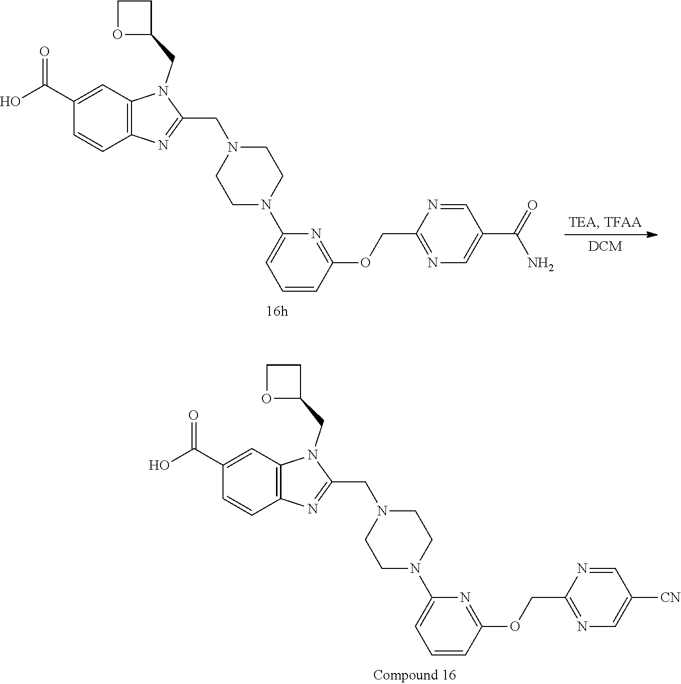 [SMILES: NC(=O)c1cnc(COc2cccc(N3CCN(Cc4nc5ccc(C(=O)O)cc5n4C[C@@H]4CCO4)CC3)n2)nc1 ; N#Cc1cnc(COc2cccc(N3CCN(Cc4nc5ccc(C(=O)O)cc5n4C[C@@H]4CCO4)CC3)n2)nc1]

Scheme 12 can be used for the synthesis of Compound 16. Detailed procedures are described in Example 16.

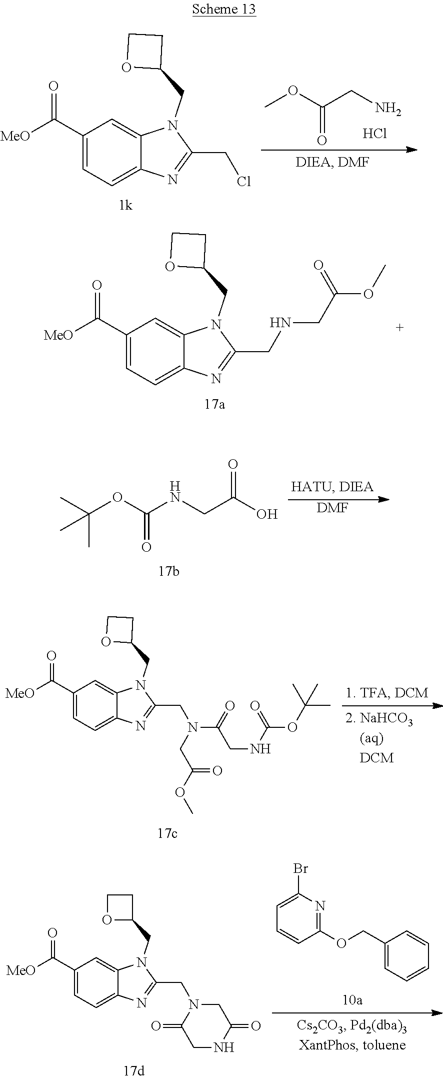

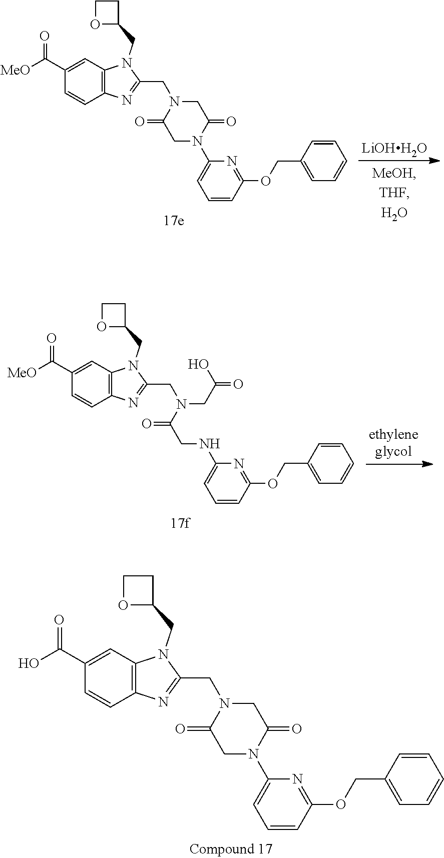

Scheme 13 can be used for the synthesis of Compound 17. Detailed procedures are described in Example 17.

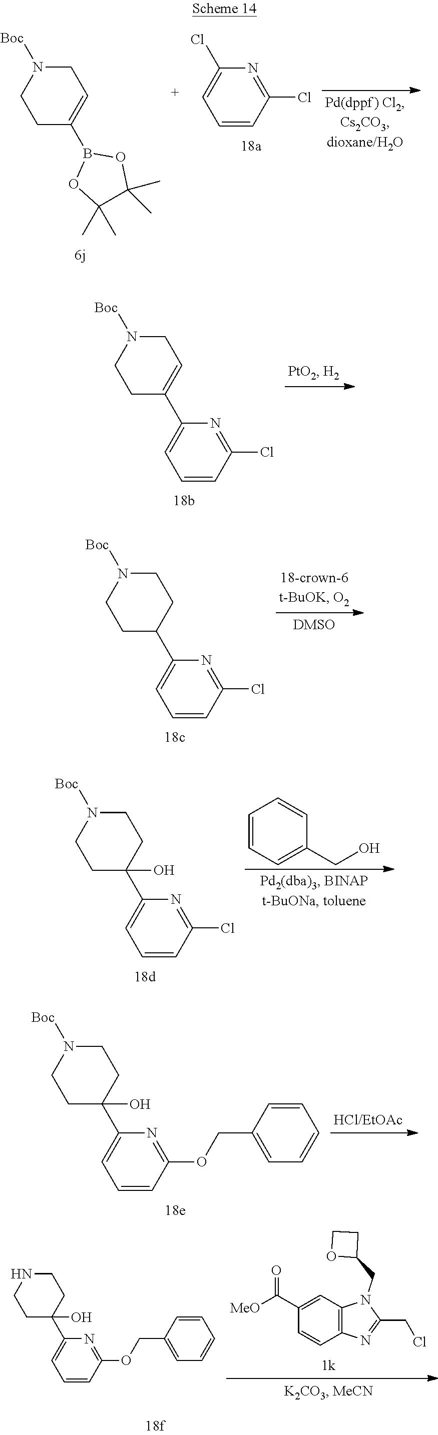

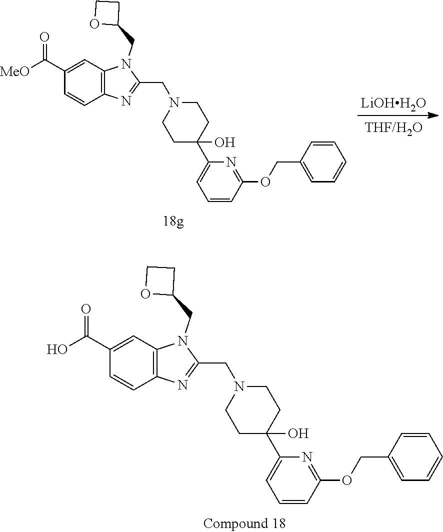 [SMILES: COC(=O)c1ccc2nc(CN3CCC(O)(c4cccc(OCc5ccccc5)n4)CC3)n(C[C@@H]3CCO3)c2c1 ; "LiOH·H2O" ; O=C(O)c1ccc2nc(CN3CCC(O)(c4cccc(OCc5ccccc5)n4)CC3)n(C[C@@H]3CCO3)c2c1]

Scheme 14 can be used for the synthesis of Compound 18. Detailed procedures are described in Example 18.

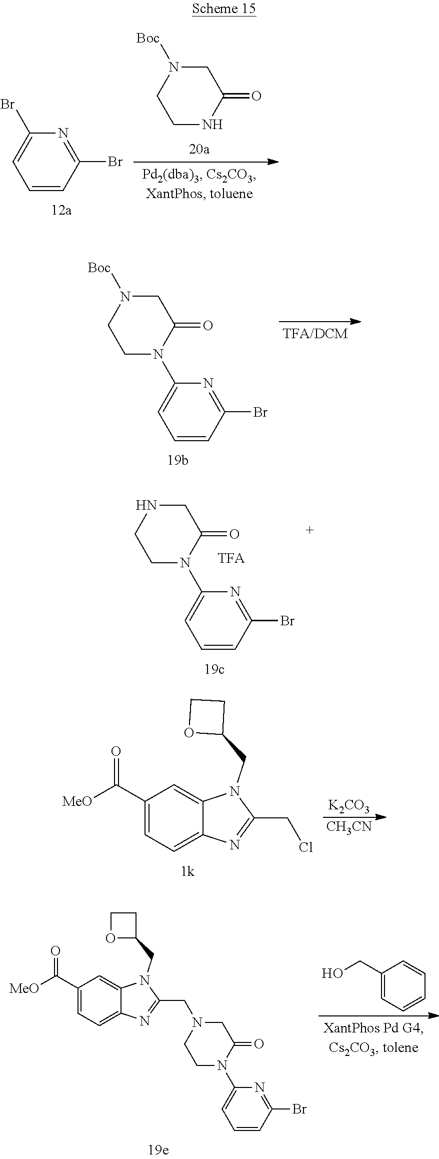

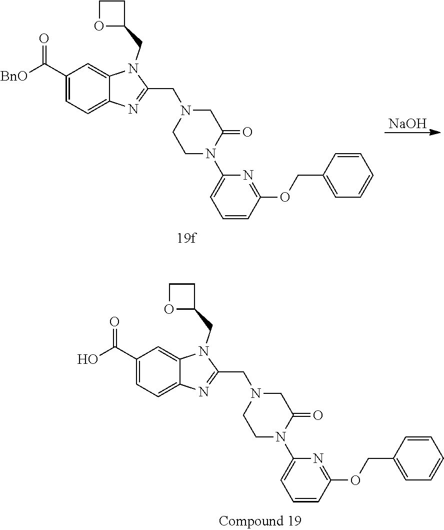

Scheme 15 can be used for the synthesis of Compound 19. Detailed procedures are described in Example 19.

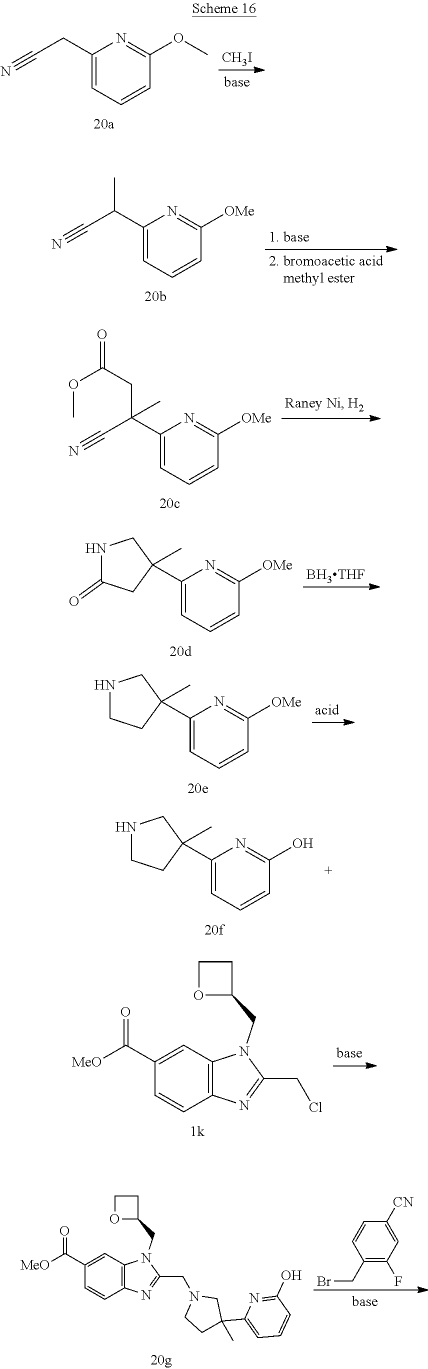

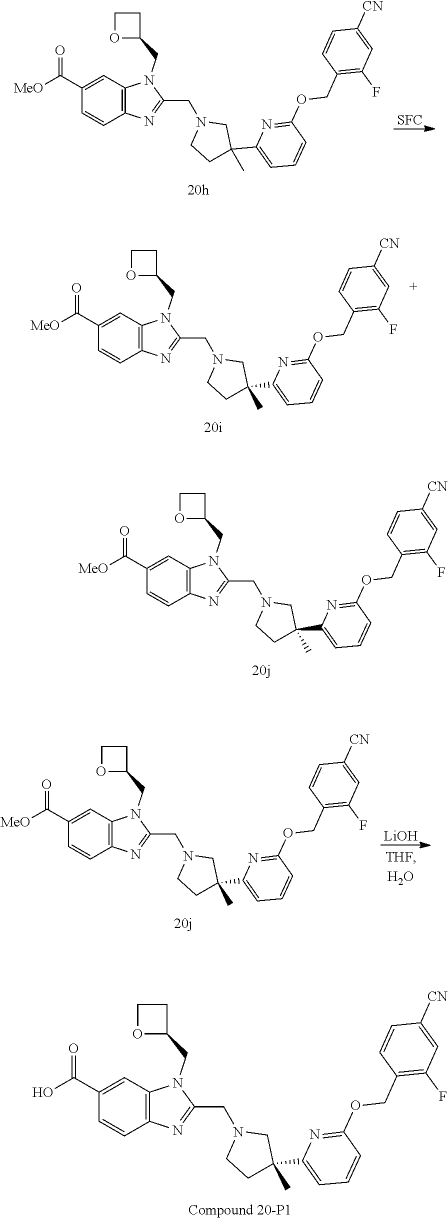

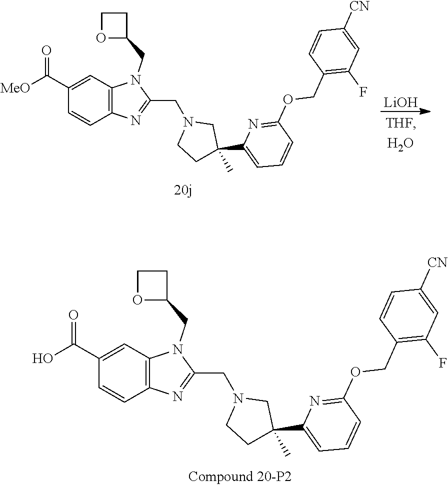

Scheme 16 can be used for the synthesis of Compound 20. Detailed procedures are described in Example 20.

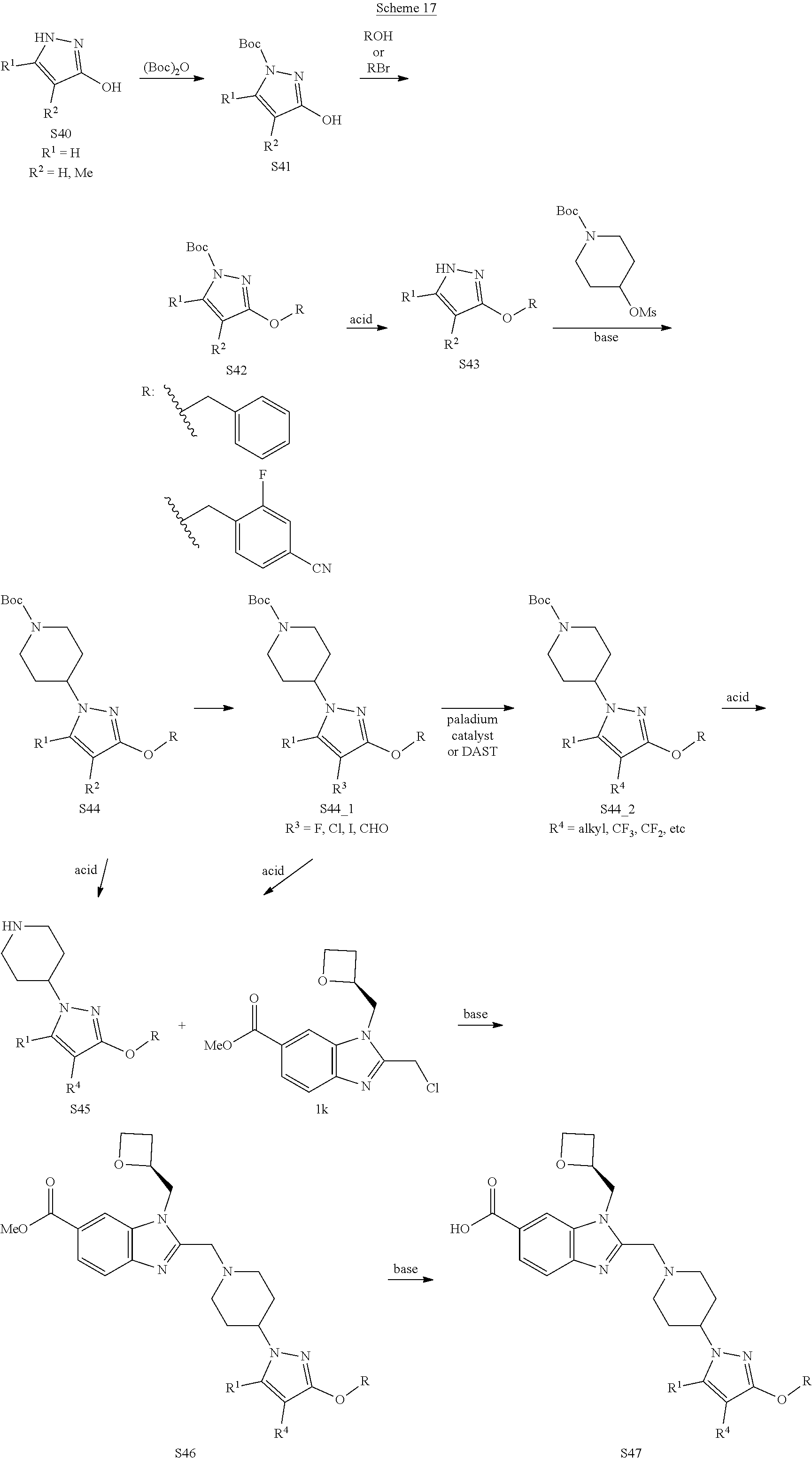

Compounds of formula (S47) may be prepared by general synthetic methods as shown in Scheme 17. Treatment of pyrazol (S40) with di-tert-butyl dicarbonate in a suitable solvent such as, but not limited to, dichloromethane with a base such as, but not limited to, triethylamine at room temperature, can readily produce Compounds of formula (S41). The Compounds of formula (S42) can be prepared from the hydroxyl pyrazole (S41) upon treatment with benzyl bromide in a suitable solvent such as, but not limited to, dimethyl formamide with a base such as, but not limited to, potassium carbonate, in the presence of salt such as, but not limited to, sodium iodide at an elevated temperature. The N-Boc (S42) with acid such as, but not limited to, trifluoroacetic acid and organic solvents such as, but not limited to, dichloromethane yields formula (S43). Treatment of pyrazol (S43) with tert-butyl 4-((methylsulfonyl)oxy)piperidine-1-carboxylate or tert-butyl 4-hydroxypiperidine-1-carboxylate in a suitable solvent such as, but not limited to, dimethyl formamide with a base such as, but not limited to, cesium carbonate at an elevated temperature, can readily produce Compounds of formula (S44). The Compounds of formula (S44_1) can be prepared from compounds of formula (S44) upon treatment with N-Chlorosuccinimide (NCS), select-F or DMF-POCl3 in a suitable solvent such as, but not limited to, chloroform at an elevated temperature. The Compounds of formula (S44_2) can be prepared from compounds of formula (S44_1) upon treatment with paladium catalyst or DAST in a suitable solvent such as, but not limited to, dioxane at an elevated temperature. The N-Boc (S44_2) with acid such as, but not limited to, trifluoroacetic acid and organic solvents such as, but not limited to, dichloromethane yields formula (S45). Compounds of formula (S46) can be prepared from the benzyl chloride (1k) upon treatment with amine (S45) under base such as, but not limited to, potassium carbonate. Treatment of the ester (S46) with hydroxide sources such as, but not limited to, lithium hydroxide in the presence of water and organic solvents such as, but not limited to, methanol and/or tetrahydrofuran yields carboxylic acid of formula (S47).

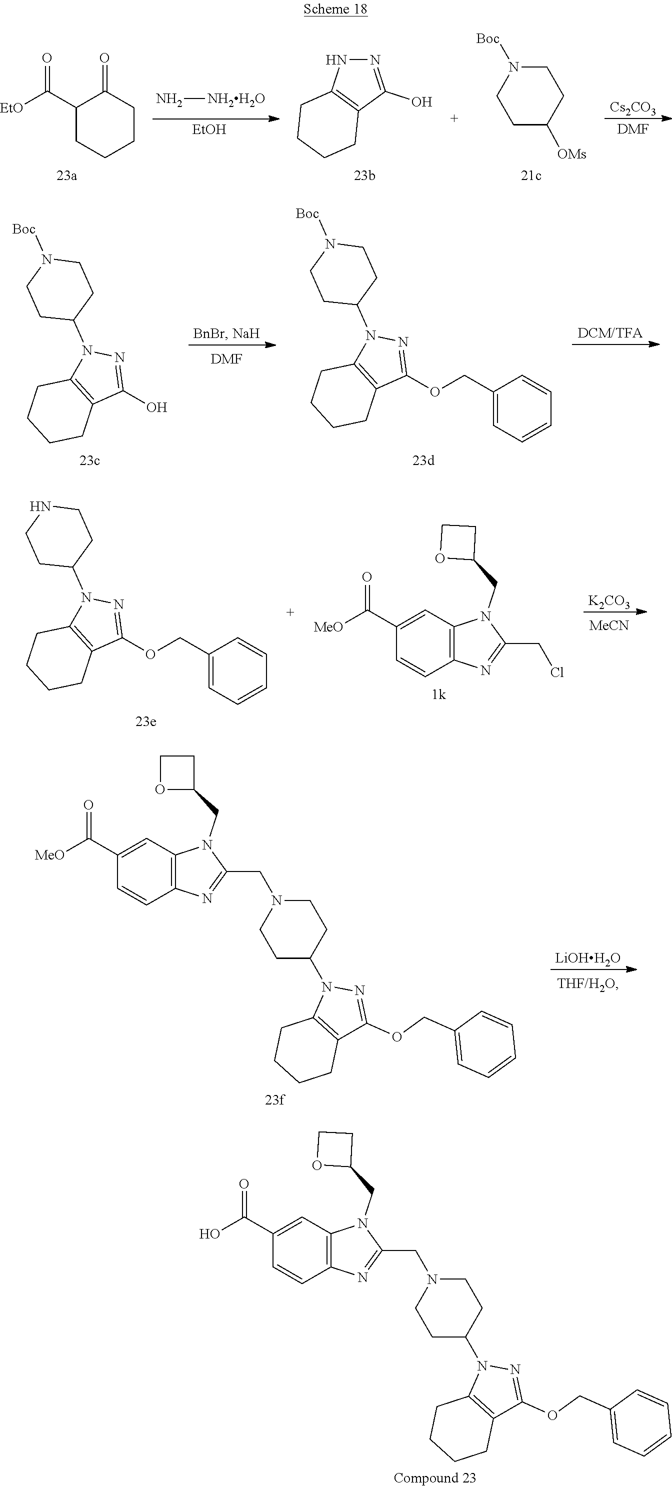

Scheme 18 can be used for the synthesis of Compound 23. Detailed procedures are described in Example 23.

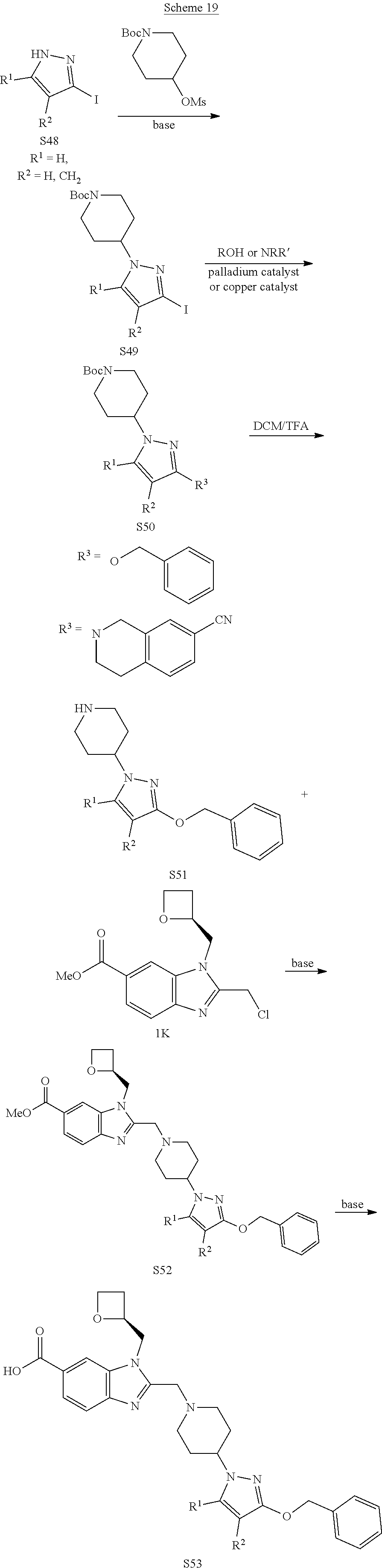

Compounds of formula (S53) may be prepared by general synthetic methods as shown in Scheme 19. Treatment of pyrazol (S48) with tert-butyl 4-((methylsulfonyl)oxy)piperidine-1-carboxylate in a suitable solvent such as, but not limited to, dimethyl formamide with a base such as, but not limited to, cesium carbonate at an elevated temperature, can readily produce Compounds of formula (S49). The Compounds of formula (S50) can be prepared from the iodopyrazole (S49) upon treatment with an alcohol or amine under palladium catalyst conditions such as, but not limited to, XPhos Pd G3 in an inorganic base such as, but not limited to, sodium 2-methylpropan-2-olate in an organic solvent such as, but not limited to, toluene at an elevated temperature. Treatment of the N-Boc (S50) with acid such as, but not limited to, trifluoroacetic acid and organic solvents such as, but not limited to, dichloromethane yields formula (S51). Compounds of formula (S52) can be prepared from the benzyl chloride (1k) upon treatment with amine (S51) under base such as, but not limited to, potassium carbonate. Treatment of the ester (S52) with hydroxide sources such as, but not limited to, lithium hydroxide in the presence of water and organic solvents such as, but not limited to, methanol and/or tetrahydrofuran yields carboxylic acid of formula (S53).

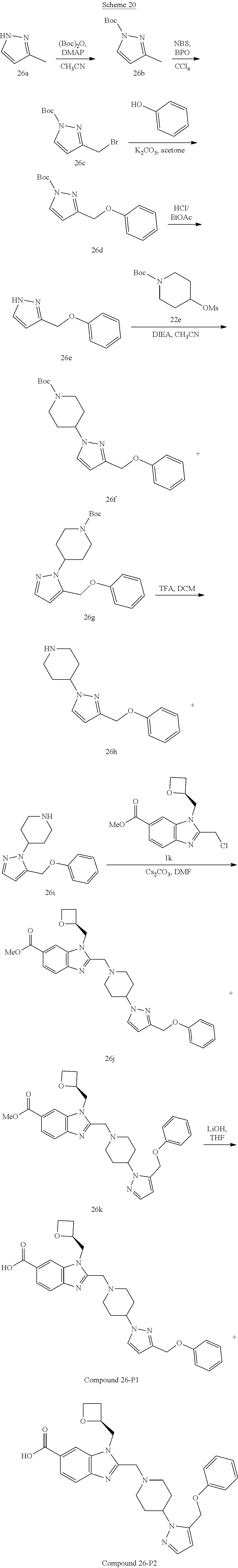

Scheme 20 can be used for the synthesis of Compound 26. Detailed procedures are described in Example 26.

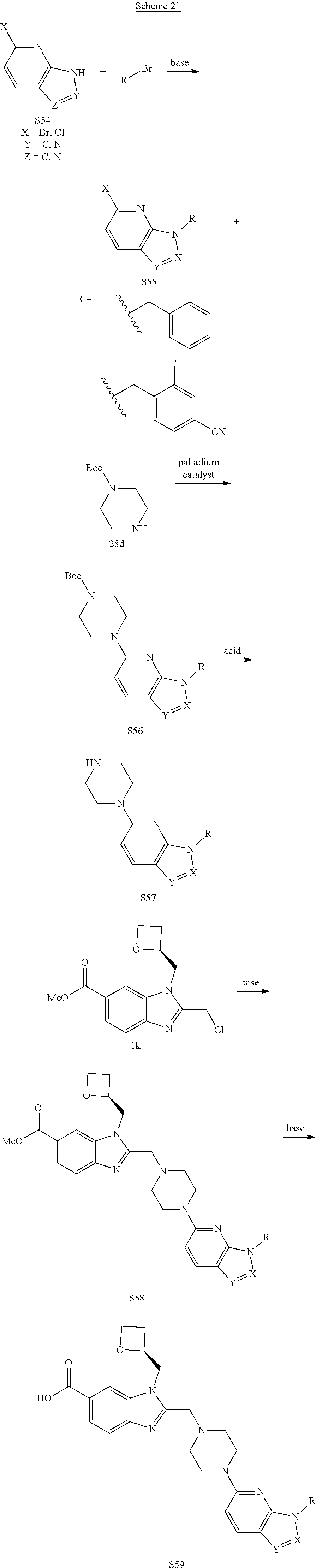

Compounds of formula (S59) may be prepared by general synthetic methods as shown in Scheme 21. Treatment of S54 with various primary Benzyl bromide in a suitable solvent such as, but not limited to, acetonitrile with a base such as, but not limited to, cesium carbonate at an elevated temperature, can readily produce Compounds of formula (S55). The Compounds of formula (S56) can be prepared from the halide (S55) upon treatment with a piperazine under palladium catalyst conditions such as, but not limited to, Tris(dibenzylideneacetone)dipalladium(O) in the presence of ligand such as, but not limited to, 2-(Dicyclohexylphosphanyl)-2,4,6-tris(isopropyl)biphenyl and an inorganic base such as, but not limited to, sodium 2-methylpropan-2-olate in an organic solvent such as, but not limited to, toluene at an elevated temperature. Treatment of the N-Boc (S56) with acid such as, but not limited to, trifluoroacetic acid and organic solvents such as, but not limited to, dichloromethane yields formula (S57). Compounds of formula (S58) can be prepared from the benzyl chloride (1k) upon treatment with amine (S57) under base such as, but not limited to, potassium carbonate. Treatment of the ester (S58) with hydroxide sources such as, but not limited to, lithium hydroxide in the presence of water and organic solvents such as, but not limited to, methanol and/or tetrahydrofuran yields carboxylic acid of formula (S59).

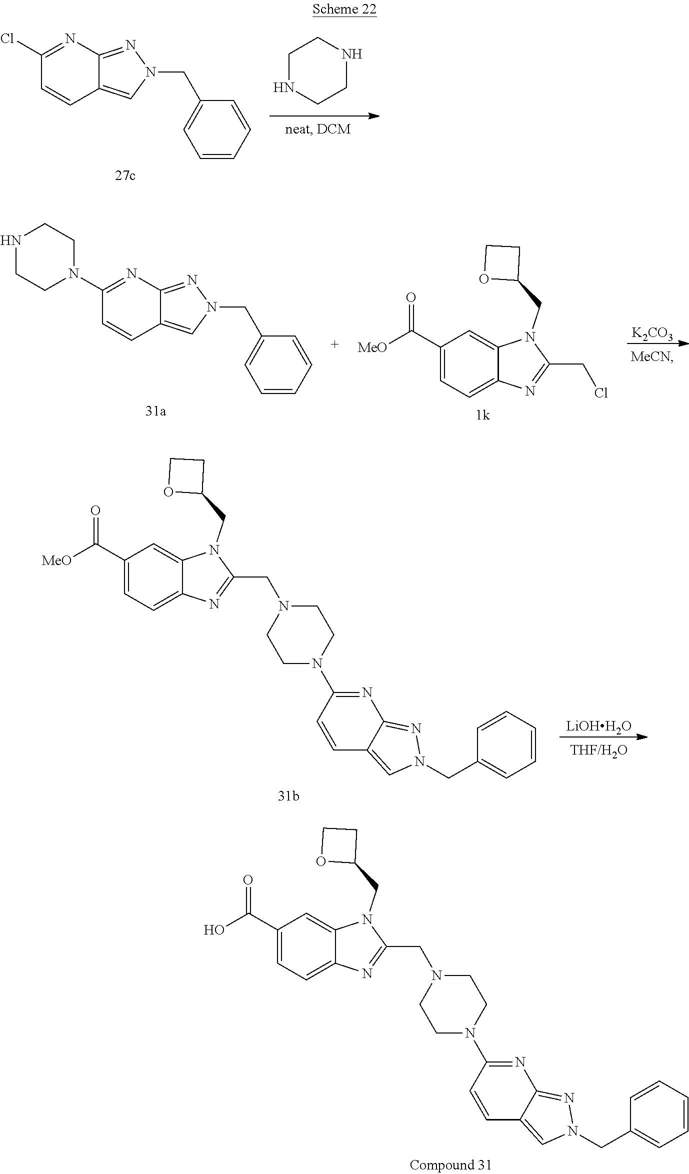

Scheme 22 can be used for the synthesis of Compound 31. Detailed procedures are described in Example 31.

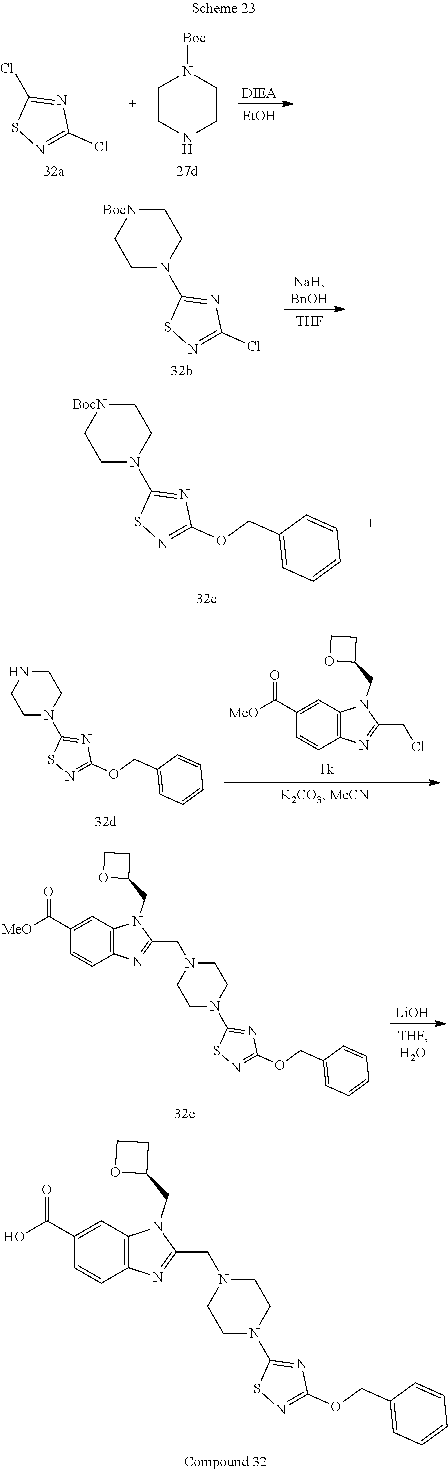

Scheme 23 can be used for the synthesis of Compound 32. Detailed procedures are described in Example 32.

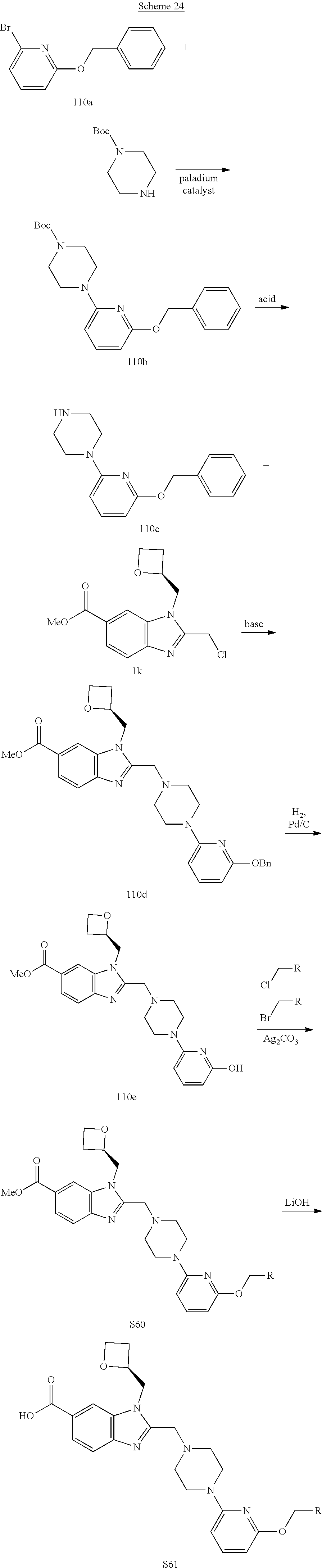

Compounds of formula (S61) may be prepared by general synthetic methods as shown in Scheme 24. The preparation of intermediate 110e please consult the procedure of Example 110. The Compounds of formula (S60) can be prepared from the pyridone (110e) upon treatment with benzyl bromide or chloride in a suitable solvent such as, but not limited to, toluene with a base such as, but not limited to, silver carbonate, at an elevated temperature. Treatment of the ester (S60) with hydroxide sources such as, but not limited to, lithium hydroxide in the presence of water and organic solvents such as, but not limited to, methanol and/or tetrahydrofuran yields carboxylic acid of formula (S61).

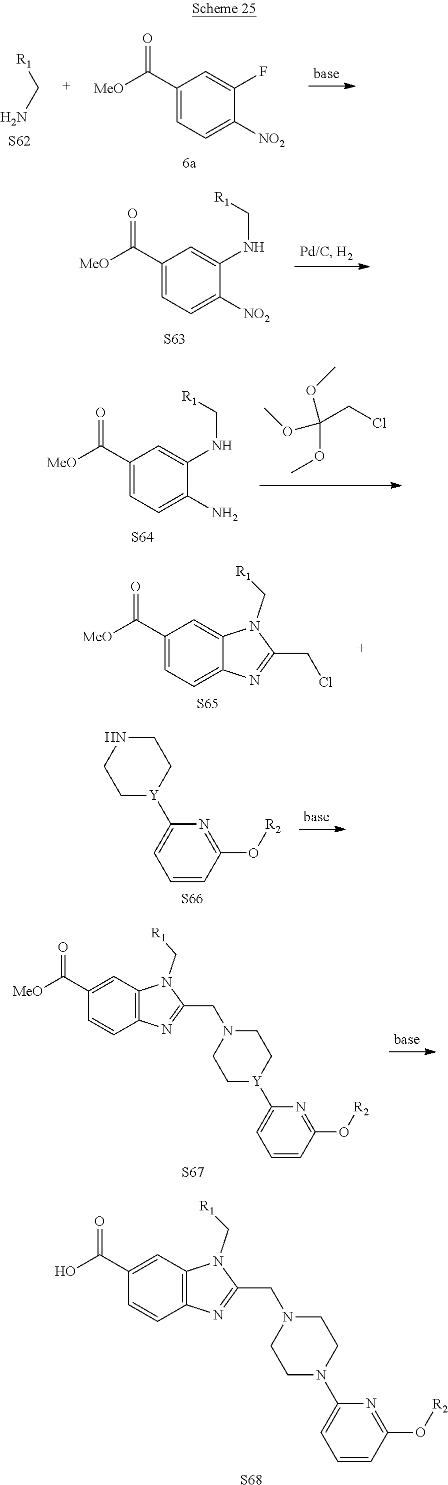

Compounds of formula (S68) may be prepared by general synthetic methods as shown in Scheme 25. Treatment of amine (S62) with Paranitrofluorobenzene in a suitable solvent such as, but not limited to, tetrahydrofuran with a base such as, but not limited to, triethylamine an elevated temperature, can readily produce Compounds of formula (S63). The Compounds of formula (S64) can be prepared from the ortho-nitroaniline (S63) upon treatment with palladium catalyst and hydrogen in a suitable solvent such as, but not limited to, methanol at room temperature or elevated temperature. The Compounds of benzimidazole (S65) can be prepared from compounds (S64) upon treatment with 2-chloro-1,1,1-trimethoxyethane in a suitable solvent such as, but not limited to, toluene at an elevated temperature. Treatment of pyrazol (S65) with amine (S66) in a suitable solvent such as, but not limited to, acetonitrile with a base such as, but not limited to, potassium carbonate at an elevated temperature, can readily produce Compounds of formula (S67). Treatment of the ester (S67) with hydroxide sources such as, but not limited to, lithium hydroxide in the presence of water and organic solvents such as, but not limited to, methanol and/or tetrahydrofuran yields carboxylic acid of formula (S68).

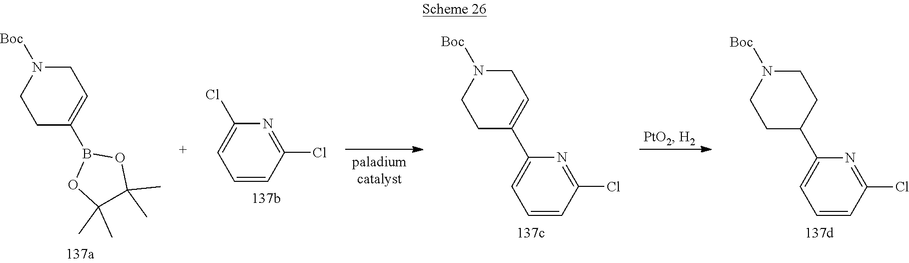 [SMILES: CC(C)(C)OC(=O)N1CC=C(B2OC(C)(C)C(C)(C)O2)CC1 ; Clc1cccc(Cl)n1 ; CC(C)(C)OC(=O)N1CC=C(c2cccc(Cl)n2)CC1 ; CC(C)(C)OC(=O)N1CCC(c2cccc(Cl)n2)CC1]

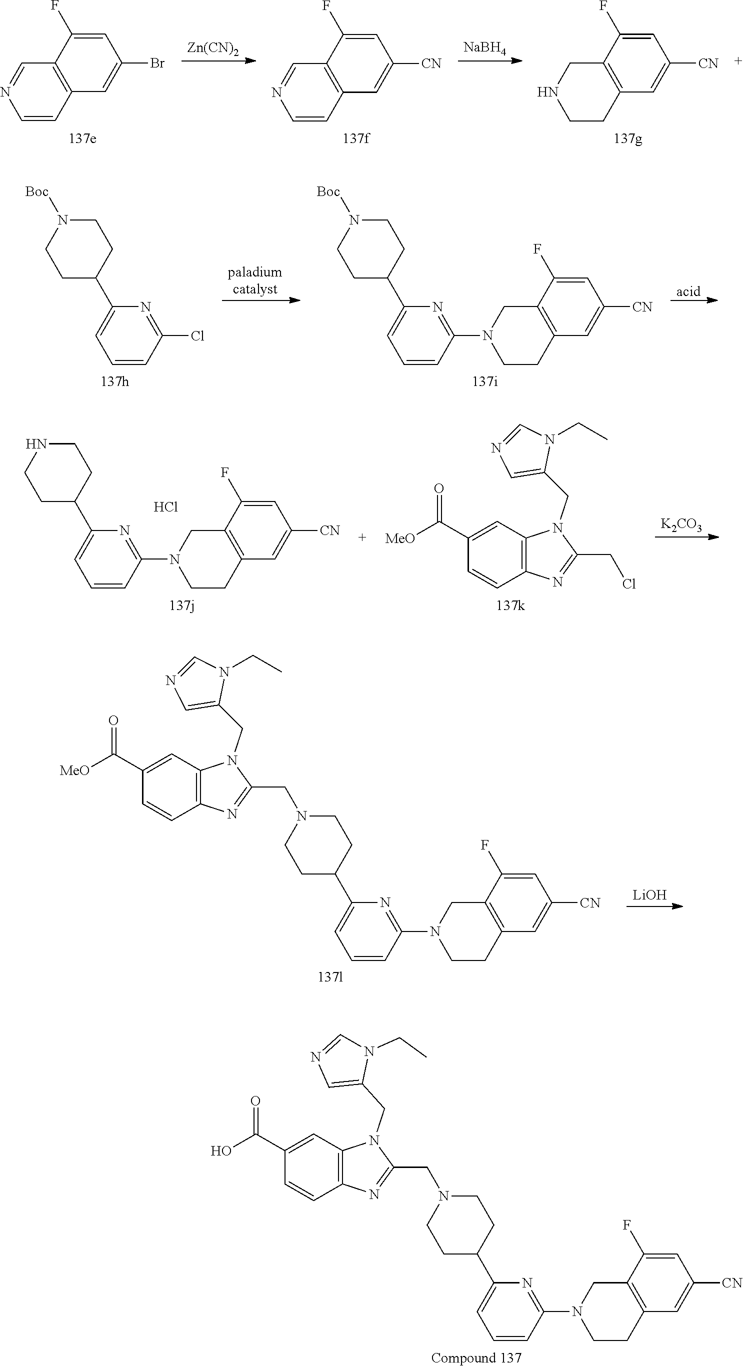

Scheme 26 can be used for the synthesis of Compound 137. Detailed procedures are described in Example 137.

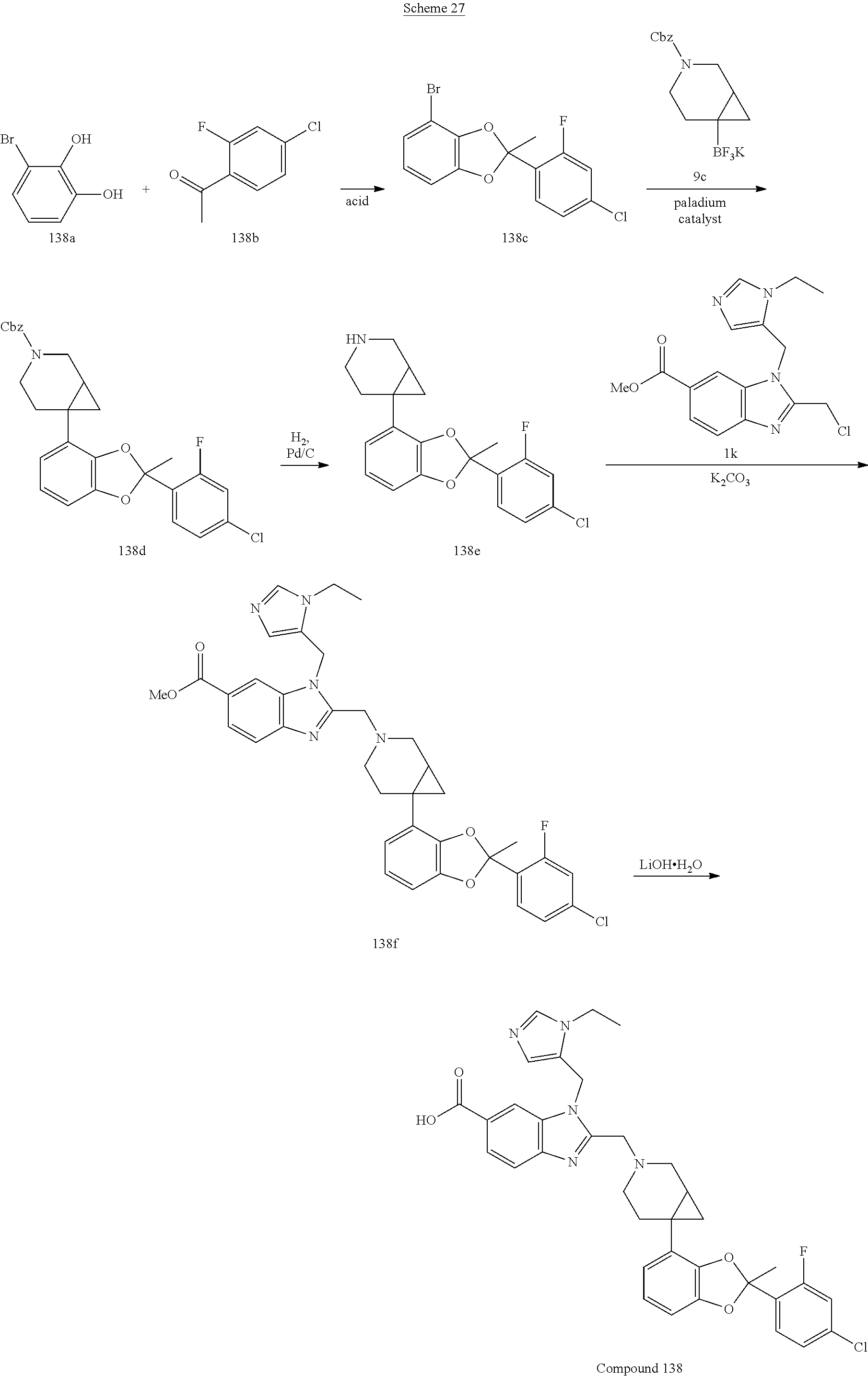

Scheme 27 can be used for the synthesis of Compound 138. Detailed procedures are described in Example 138.

### Part II. Synthetic Examples

### Example 1 (General Procedure A)

### (S)-2-((4-(6-(cyclohexylmethoxy)pyridin-2-yl)piperazin-1-yl)methyl)-1-(oxetan-2-ylmethyl)-1H-benzo[d]imidazole-6-carboxylic acid

The title compound was prepared according to Scheme 1. This General Procedure A exemplifies Scheme 1 and provides particular synthetic details as applied to the title compound.

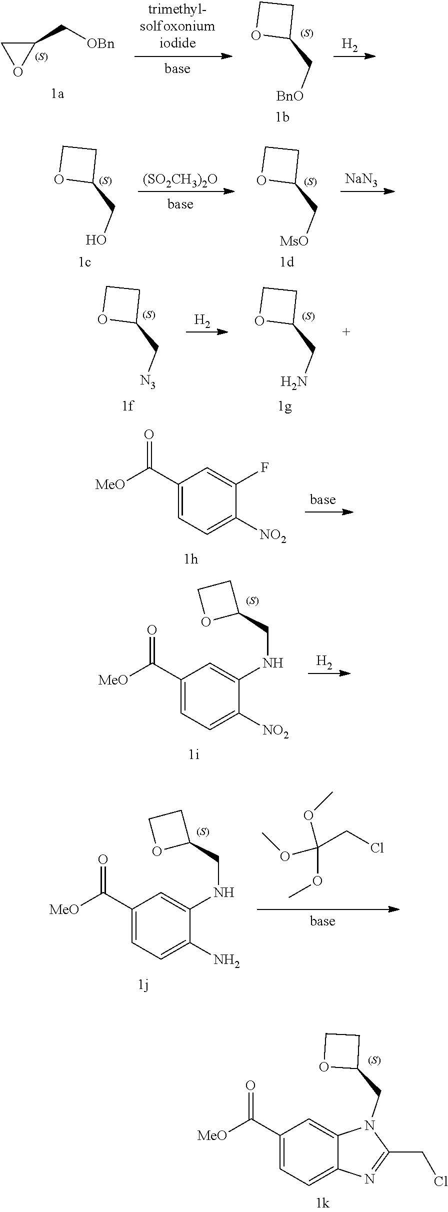

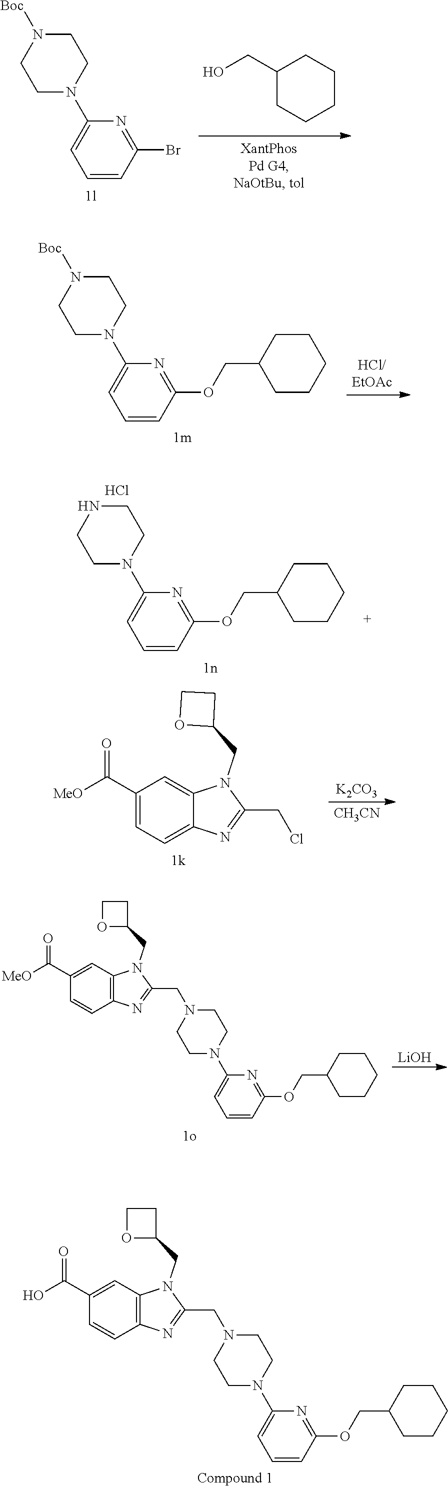

### (S)-2-((benzyloxy)methyl)oxetane (1b)

To a solution of t-BuOK (54.67 g, 487.21 mmol, 2 eq) in t-BuOH (450 mL) was added Trimethylsulfoxonium iodide (107.22 g, 487.21 mmol, 2 eq) at 25° C. The mixture was heated to 60° C., and stirred for 30 min. Then (S)-2-((benzyloxy)methyl)oxirane (1a, 40 g, 243.60 mmol, 1 eq) was added in the mixture. Heat is generated during the reaction (˜10° C.). The mixture was heated to 80° C. and stirred for another 2 hours. TLC (petroleum ether:ethyl acetate=2:1) showed 1b was consumed and one new spot was formed. The reaction mixture was filtered and the filtrate was partitioned between petroleum ether (300 mL) and H2O (300 mL). The aqueous phase was extracted with petroleum ether (100 mL*2). The combined organic layers were washed with brine (100 mL), dried over with anhydrous Na2SO4, filtered and concentrated in vacuo. The residue was purified by silica gel column chromatography (petroleum ether:ethyl acetate=92:8 to 9:1) to give 1b as a yellow oil. 1H NMR (400 MHz, CDCl3-d) δ 7.40-7.29 (m, 5H), 5.06-4.91 (m, 1H), 4.72-4.55 (m, 4H), 3.73-3.57 (m, 2H), 2.74-2.53 (m, 2H).

### (S)-oxetan-2-ylmethanol (1c)

To a solution of (S)-2-((benzyloxy)methyl)oxetane (1b, 5 g, 28.05 mmol, 1 eq) in THF (100 mL) was added Pd(OH)2 (500.00 mg, 356.04 umol, 10% purity). The mixture was stirred at 45° C. for 32 hours under H2 (50 psi). TLC (petroleum ether:ethyl acetate=0:1) showed one new spot was formed. The reaction mixture was filtered and the filtrate was used into the next step without work up. Compound 1c was obtained as a Colorless Liquid.

### (S)-oxetan-2-ylmethyl methanesulfonate (1d)

To a solution of (S)-oxetan-2-ylmethanol (1c, 2.47 g, 28.03 mmol, 1 eq) in THF (85 mL) was added Et3N (7.09 g, 70.09 mmol, 9.76 mL, 2.5 eq) at 0° C. A solution of methylsulfonyl methanesulfonate (7.33 g, 42.05 mmol, 1.5 eq) in THF was added in the mixture dropwise, and the internal temperature was kept below 10° C. The mixture was stirred at 25° C. for 16 hours. TLC (petroleum ether:ethyl acetate=0:1) showed 1c was consumed and one new spot was formed. The mixture was quenched with H2O (100 mL) and extracted with DCM (100 mL*3). The combined organic layers were washed with brine (30 mL), dried over with anhydrous Na2SO4, filtered and concentrated in vacuo. The residue was purified by silica gel column chromatography (Petroleum ether:Ethyl acetate=6:4-1:1) to give 1d as a yellow oil. 1H NMR (400 MHz, CDCl3-d) δ 5.09-4.99 (m, 1H), 4.75-4.66 (m, 1H), 4.59 (td, J=6.0, 9.0 Hz, 1H), 4.37 (d, J=3.8 Hz, 2H), 3.11 (s, 3H), 2.83-2.73 (m, 1H), 2.69-2.58 (m, 1H).

### (S)-2-(azidomethyl)oxetane (1f)

To a solution of (S)-oxetan-2-ylmethyl methanesulfonate (i d, 1.37 g, 8.24 mmol, 1 eq) in DMF (10 mL) was added NaN3 (819.92 mg, 12.61 mmol, 1.53 eq). The mixture was stirred at 80° C. for 6 hours. TLC (petroleum ether:ethyl acetate=0:1) showed 1d was consumed, and one new spot was formed. The reaction mixture was filtered at 0° C., and the filtrate was used in the next step without work up. 1f in DMF was obtained as a colorless liquid.

### (S)-oxetan-2-ylmethanamine (1g)

A mixture of (S)-2-(azidomethyl)oxetane (1f, 932 mg, 8.24 mmol, 1 eq), Pd/C (310.67 mg, 262.39 umol, 10% purity) in DMF (10 mL) and THF (20 mL) at 0° C. was degassed and purged with H2 3 times, and then the mixture was stirred at 40° C. for 16 hours under H2 (15 psi). TLC (petroleum ether:ethyl acetate=1:1) showed 1f was consumed and one new spot was formed. The reaction mixture was filtered at 0° C., and the filtrate was used into the next step without work up. Compound 1g in DMF and THF was obtained as a colorless liquid.

### (S)-methyl 4-nitro-3-((oxetan-2-ylmethyl)amino)benzoate (1i)

To a solution of methyl 3-fluoro-4-nitrobenzoate (1 h, 1.2 g, 6.03 mmol, 1 eq) and (S)-oxetan-2-ylmethanamin (1g, 698.24 mg, 8.01 mmol, 1.33 eq) in THF (30 mL) and DMF (10 mL) was added TEA (1.22 g, 12.05 mmol, 1.68 mL, 2 eq). The mixture was stirred at 60° C. for 3 hours. TLC (Petroleum ether:Ethyl acetate=1:1) showed 1g was consumed and one new spot was formed. The mixture was quenched with water (40 mL) and extracted with ethyl acetate (40 mL*3). The combined organic layers were washed with brine (20 mL*3), dried over with anhydrous Na2SO4, filtered and concentrated in vacuo. The crude product was purified by silica gel column chromatography (Petroleum ether:Ethyl acetate=10:1˜1:1) to give 1i as a yellow solid. 1H NMR (400 MHz, CDCl3-d) δ 8.29 (br s, 1H), 8.35-8.23 (m, 1H), 8.21-8.13 (m, 1H), 7.56 (s, 1H), 7.63-7.53 (m, 1H), 7.19 (br d, J=8.8 Hz, 1H), 5.18-5.06 (m, 1H), 4.74-4.66 (m, 1H), 4.62-4.52 (m, 1H), 3.92-3.83 (m, 3H), 3.63-3.54 (m, 2H), 2.78-2.66 (m, 1H), 2.62-2.47 (m, 1H).

### (S)-methyl 4-amino-3-((oxetan-2-ylmethyl)amino)benzoate (1j)

To a solution of (S)-methyl 4-nitro-3-((oxetan-2-ylmethyl)amino)benzoate (11, 1g, 3.76 mmol, 1 eq) in THF (30 mL) was added Pd/C (444.70 mg, 375.59 umol, 10% purity). The mixture was stirred at 20° C. for 4 hours under H2. TLC (Petroleum ether:Ethyl acetate=1:1) showed 1i was consumed, and one new spot was formed. The reaction mixture was filtered and the filtrate was concentrated. The product was used into the next step without purification. 1j was obtained as yellow oil. 1H NMR (400 MHz, CDCl3-d) δ 7.52-7.47 (m, 1H), 7.39 (d, J=1.8 Hz, 1H), 6.70 (d, J=8.2 Hz, 1H), 5.12 (dq, J=3.7, 6.9 Hz, 1H), 4.80-4.71 (m, 1H), 4.62 (td, J=6.1, 9.0 Hz, 1H), 3.87 (s, 3H), 3.53-3.31 (m, 2H), 2.82-2.72 (m, 1H), 2.68-2.55 (m, 1H).

### (S)-methyl 2-(chloromethyl)-1-(oxetan-2-ylmethyl)-1H-benzo[d]imidazole-6-carboxylate (1k)

To a solution of (S)-methyl 4-amino-3-((oxetan-2-ylmethyl)amino)benzoate (1j, 880 mg, 3.72 mmol, 1 eq) and 2-chloro-1,1,1-trimethoxy-ethane (604.58 mg, 3.91 mmol, 525.72 uL, 1.05 eq) in MeCN (20 mL) was added PTSA (64.14 mg, 372.46 umol, 0.1 eq). The mixture was stirred at 60° C. for 2 hours. LCMS showed one major peak with desired mass. The reaction mixture was concentrated. The crude product was purified by silica gel column chromatography (Petroleum ether:Ethyl acetate=1:1-0:1) to give 1k as a yellow solid. 1H NMR (400 MHz, CDCl3-d) δ 8.13 (s, 1H), 8.02 (dd, J=1.4, 8.6 Hz, 1H), 7.80 (d, J=8.4 Hz, 1H), 5.22 (dq, J=2.8, 7.0 Hz, 1H), 5.04 (s, 2H), 4.68-4.60 (m, 2H), 4.57-4.51 (m, 1H), 4.35 (td, J=6.0, 9.2 Hz, 1H), 3.97 (s, 3H), 2.82-2.71 (m, 1H), 2.49-2.38 (m, 1H).

### Tert-butyl 4-(6-(cyclohexylmethoxy)pyridin-2-yl)piperazine-1-carboxylate (1m)

To a solution of tert-butyl 4-(6-bromopyridin-2-yl)piperazine-1-carboxylate (11, 400.38 mg, 3.51 mmol, 430.51 uL, 3 eq) in toluene (30 mL) was added NaOtBu (224.65 mg, 2.34 mmol, 2 eq) and XantPhos Pd G4 (112.48 mg, 116.88 umol, 0.1 eq). The mixture was stirred at 100° C. for 16 hours under Ar. TLC (Petroleum ether:Ethyl acetate=3:1) showed 11 was consumed, and one major new spot was formed. The mixture was concentrated in vacuo. The residue was diluted with water (10 mL) and extracted with ethyl acetate (40 mL*2). The combined organic layers were washed with brine (20 mL), dried over with anhydrous Na2SO4, filtered and concentrated in vacuo. The residue was purified by silica gel chromatography (Petroleum ether/Ethyl acetate=20:1 to 5:1) to give 1m as a white solid. 1H NMR (400 MHz, CDCl3-d) δ 7.40 (t, J=7.9 Hz, 1H), 6.14 (d, J=7.9 Hz, 1H), 6.10 (d, J=7.9 Hz, 1H), 4.03 (d, J=6.4 Hz, 2H), 3.52 (br dd, J=5.8, 17.4 Hz, 8H), 1.85 (br d, J=13.2 Hz, 2H), 1.79-1.73 (m, 2H), 1.70 (br d, J=11.5 Hz, 1H), 1.49 (s, 9H), 1.37-1.17 (m, 4H), 1.11-0.97 (m, 2H).

### 1-(6-(cyclohexylmethoxy)pyridin-2-yl)piperazine hydrochloride (1n)

To a solution of tert-butyl 4-(6-(cyclohexylmethoxy)pyridin-2-yl)piperazine-1-carboxylate (1m, 170 mg, 452.73 umol, 1 eq) in HCl/EtOAc (10 mL) was stirred at 15° C. for 30 min. TLC (Petroleum ether:Ethyl acetate=0:1) showed 1m was consumed, and one major new spot was formed. The mixture was concentrated in vacuo. The product was used to next step without further purification. In was obtained as a white solid. 1H NMR (400 MHz, MeOD-d4) δ 7.92-7.71 (m, 1H), 6.68 (d, J=8.4 Hz, 1H), 6.45 (d, J=8.0 Hz, 1H), 4.10 (d, J=6.4 Hz, 2H), 3.93-3.80 (m, 4H), 3.43-3.36 (m, 4H), 1.92-1.67 (m, 6H), 1.40-1.25 (m, 3H), 1.12 (br d, J=11.6 Hz, 2H).

### (S)-methyl 2-((4-(6-(cyclohexylmethoxy)pyridin-2-yl)piperazin-1-yl)methyl)-1-(oxetan-2-ylmethyl)-1H-benzo[d]imidazole-6-carboxylate (1o)

To a solution of 1-(6-(cyclohexylmethoxy)pyridin-2-yl)piperazine hydrochloride (In, 140 mg, 448.93 umol, 1 eq, HCl) and (S)-methyl 2-(chloromethyl)-1-(oxetan-2-ylmethyl)-1H-benzo[d]imidazole-6-carboxylate (1k, 172.01 mg, 583.62 umol, 1.3 eq) in MeCN (10 mL) was added K2CO3 (186.14 mg, 1.35 mmol, 3 eq). The mixture was stirred at 80° C. for 3 hours. LCMS showed 1n was consumed completely and desired mass was detected. The mixture was concentrated in vacuo. The residue was diluted with water (10 mL) and extracted with ethyl acetate (20 mL*2). The combined organic layers were washed with brine (10 mL), dried over with anhydrous Na2SO4, filtered and concentrated in vacuo. The residue was purified by silica gel column chromatography (Petroleum ether:Ethyl acetate=10:1 to 0:1) to give 1o as a brown oil. 1H NMR (400 MHz, CDCl3-d) δ 8.17 (s, 1H), 7.99 (dd, J=1.6, 8.4 Hz, 1H), 7.76 (d, J=8.4 Hz, 1H), 7.39 (t, J=8.0 Hz, 1H), 6.13 (d, J=8.0 Hz, 1H), 6.08 (d, J=7.8 Hz, 1H), 5.25 (br s, 1H), 4.79-4.60 (m, 3H), 4.44-4.35 (m, 1H), 4.04-3.98 (m, 4H), 3.96 (s, 3H), 3.51 (br s, 4H), 2.80-2.70 (m, 1H), 2.66 (br t, J=4.8 Hz, 4H), 2.53-2.42 (m, 1H), 1.88-1.63 (m, 6H), 1.26-1.10 (m, 3H), 1.08-0.95 (m, 2H).

### (S)-2-((4-(6-(cyclohexylmethoxy)pyridin-2-yl)piperazin-1-yl)methyl)-1-(oxetan-2-ylmethyl)-1H-benzo[d]imidazole-6-carboxylic acid (Compound 1)

To a solution of (S)-methyl 2-((4-(6-(cyclohexylmethoxy)pyridin-2-yl)piperazin-1-yl)methyl)-1-(oxetan-2-ylmethyl)-1H-benzo[d]imidazole-6-carboxylate (10, 80 mg, 149.91 umol, 1 eq) in THF (2 mL), MeOH (2 mL) and H2O (2 mL) was added LiOH·H2O (31.45 mg, 749.54 umol, 5 eq). The mixture was stirred at 20° C. for 16 hours. LCMS showed 1o was consumed completely and desired mass was detected. 1 M citric acid was added to the reaction mixture drop-wise until pH=6. The aqueous phase was extracted with ethyl acetate (20 mL*3) and H2O (10 mL). The combined organic layers were dried over with anhydrous Na2SO4, filtered and concentrated in vacuo. The mixture was purified by pre-HPLC (Column: Waters Xbridge BEH C18 100*30 mm*10 um; mobile phase: [water (10 mM NH4HCO3)-ACN]; B %: 30%-55%, 8 min) to give Compound 1 as a white solid. MS mass calculated for [M+H]+ (C29H37N5O4) requires m/z 520.2, LCMS found m/z 520.2. 1H NMR (400 MHz, MeOD-d4) δ 8.19 (s, 1H), 7.95 (d, J=8.3 Hz, 1H), 7.59 (d, J=8.4 Hz, 1H), 7.40 (t, J=7.9 Hz, 1H), 6.23 (d, J=8.1 Hz, 1H), 6.02 (d, J=7.9 Hz, 1H), 5.29 (br s, 1H), 4.92 (br s, 1H), 4.77-4.69 (m, 1H), 4.66-4.59 (m, 1H), 4.47 (td, J=6.0, 9.0 Hz, 1H), 4.09-3.88 (m, 4H), 3.52 (br s, 4H), 2.86-2.72 (m, 1H), 2.62 (br s, 5H), 1.92-1.62 (m, 6H), 1.38-1.17 (m, 3H), 1.04 (br d, J=11.6 Hz, 2H)

### Example 2 (General Procedure B)

### (S)-2-((4-(6-(7-cyano-4,5-dihydro-1H-benzo[d]azepin-3(2H)-yl)pyridin-2-yl)piperazin-1-yl)methyl)-1-(oxetan-2-ylmethyl)-1H-benzo[d]imidazole-6-carboxylic acid

The title compound was prepared according to Scheme 2. This General Procedure B exemplifies Scheme 2 and provides particular synthetic details as applied to the title compound.

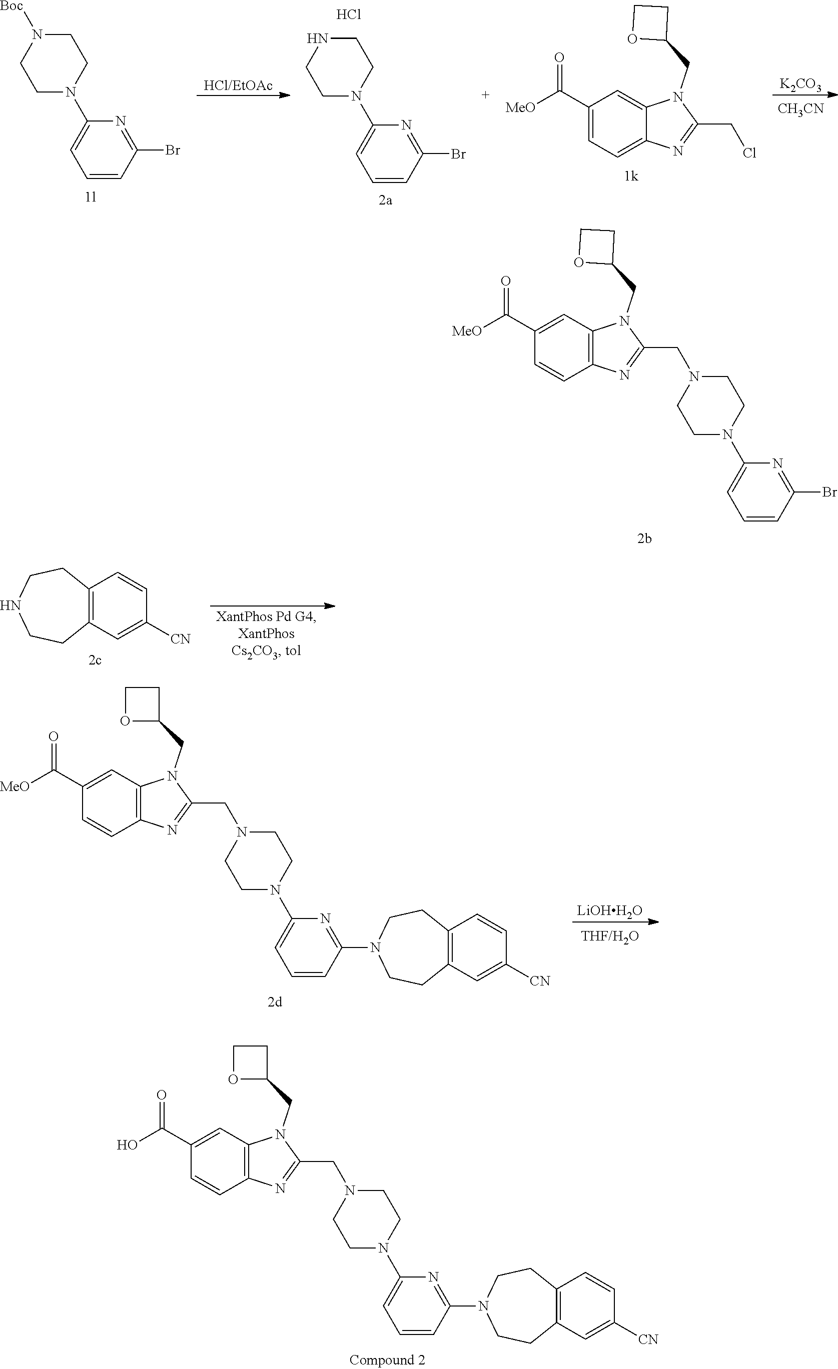

### 1-(6-bromopyridin-2-yl)piperazine hydrochloride (2a)

A solution of tert-butyl 4-(6-bromo-2-pyridyl)piperazine-1-carboxylate (11, 4.3 g, 12.56 mmol, 1 eq) in HCl/EtOAc (50 mL) was stirred at 15° C. for 30 minutes. TLC (Petroleum ether:Ethyl acetate=3:1) showed 11 was consumed completely, and one major new spot was formed. The mixture was concentrated in vacuo. The product was used to next step without further purification. 1H NMR (400 MHz, DMSO-d6) δ 9.40 (br s, 2H), 7.51 (dd, J=7.6, 8.4 Hz, 1H), 6.93-6.86 (m, 2H), 3.81-3.66 (m, 4H), 3.15 (br s, 4H).

### (S)-methyl 2-((4-(6-bromopyridin-2-yl)piperazin-1-yl)methyl)-1-(oxetan-2-ylmethyl)-1H-benzo[d]imidazole-6-carboxylate (2b)

To a solution of 1-(6-bromopyridin-2-yl)piperazine hydrochloride (2a, 3.5 g, 12.56 mmol, 1 eq) and methyl 2-(chloromethyl)-3-[[(2S)-oxetan-2-yl]methyl]benzimidazole-5-carboxylate (1k, 4.26 g, 14.45 mmol, 1.15 eq) in CH₃CN (50 mL) was added K2CO3 (5.21 g, 37.69 mmol, 3 eq). The mixture was stirred at 80° C. for 5 hours. LCMS showed 2a was consumed completely and desired mass was detected. The mixture was concentrated in vacuo. The residue was diluted with water (50 mL) and extracted with ethyl acetate (60 mL*2). The combined organic layers were washed with brine (30 mL), dried over with anhydrous Na2SO4, filtered and concentrated in vacuo. The residue was purified by silica gel column chromatography (Petroleum ether:Ethyl acetate=10:1 to 0:1) to give 2b as a white solid. 1H NMR (400 MHz, CDCl3-d) δ 8.17 (s, 1H), 7.98 (dd, J=1.3, 8.6 Hz, 1H), 7.76 (d, J=8.4 Hz, 1H), 7.31-7.28 (m, 1H), 7.26 (s, 1H), 6.76 (d, J=7.6 Hz, 1H), 6.50 (d, J=8.4 Hz, 1H), 5.31-5.14 (m, 1H), 4.81-4.55 (m, 3H), 4.39 (td, J=6.0, 9.4 Hz, 1H), 4.01 (d, J=2.0 Hz, 2H), 3.96 (s, 3H), 3.63-3.44 (m, 4H), 2.80-2.69 (m, 1H), 2.64 (t, J=5.1 Hz, 4H), 2.51-2.38 (m, 1H).

### (S)-methyl 2-((4-(6-(7-cyano-4,5-dihydro-1H-benzo[d]azepin-3(2H)-yl)pyridin-2-yl)piperazin-1-yl)methyl)-1-(oxetan-2-ylmethyl)-1H-benzo[d]imidazole-6-carboxylate (2d)

A mixture of (S)-methyl 2-((4-(6-bromopyridin-2-yl)piperazin-1-yl)methyl)-1-(oxetan-2-ylmethyl)-1H-benzo[d]imidazole-6-carboxylate (2b, 60 mg, 119.91 umol, 1 eq), 2,3,4,5-tetrahydro-1H-benzo[d]azepine-7-carbonitrile (2c, 25.81 mg, 149.88 umol, 1.25 eq), XantPhos Pd G4 (11.54 mg, 11.99 umol, 0.1 eq), Xantphos (10.41 mg, 17.99 umol, 0.15 eq) and Cs2CO3 (195.34 mg, 599.54 umol, 5 eq) in toluene (2 mL) was degassed and purged with N2 3 times, and then the mixture was stirred at 100° C. for 16 hours under N2. LCMS showed 2b was consumed and desired mass was detected. The reaction mixture was concentrated under reduced pressure. The residue was purified by prep-TLC (SiO2, Ethyl acetate:Methanol=20:1) to give 2d as a white solid. 1H NMR (400 MHz, CDCl3-d) δ 8.18 (s, 1H), 8.05-7.94 (m, 1H), 7.83-7.72 (m, 1H), 7.48-7.30 (m, 3H), 7.20 (d, J=7.6 Hz, 1H), 6.13-5.92 (m, 2H), 5.25 (br s, 1H), 4.80-4.69 (m, 2H), 4.69-4.58 (m, 1H), 4.41 (td, J=6.0, 9.0 Hz, 1H), 4.02 (s, 2H), 3.96 (s, 3H), 3.87-3.78 (m, 4H), 3.56-3.47 (m, 4H), 3.08-2.92 (m, 4H), 2.85-2.71 (m, 1H), 2.67 (br s, 4H), 2.57-2.38 (m, 1H).

### (S)-2-((4-(6-(7-cyano-4,5-dihydro-1H-benzo[d]azepin-3(2H)-yl)pyridin-2-yl)piperazin-1-yl)methyl)-1-(oxetan-2-ylmethyl)-1H-benzo[d]imidazole-6-carboxylic acid (Compound 2)

To a solution of (S)-methyl 2-((4-(6-(7-cyano-4,5-dihydro-1H-benzo[d]azepin-3(2H)-yl)pyridin-2-yl)piperazin-1-yl)methyl)-1-(oxetan-2-ylmethyl)-1H-benzo[d]imidazole-6-carboxylate (2d, 50 mg, 84.50 umol, 1 eq) in THF (3.5 mL) and H2O (1.5 mL) was added LiOH·H2O (10 mg, 238.30 umol, 2.82 eq). The mixture was stirred at 15° C. for 16 hours. LCMS showed 2d was consumed and desired mass was detected. 1 M citric acid was added to the reaction mixture drop-wise until pH=6. The aqueous phase was extracted with ethyl acetate (20 mL*3) and H2O (10 mL). The combined organic layers were dried over with anhydrous Na2SO4, filtered and concentrated in vacuo. The residue was purified by prep-HPLC (neutral condition; column: Phenomenex Gemini-NX C18 75*30 mm*3 um; mobile phase: [water (10 mM NH4HCO3)-ACN]; B %: 21%-41%, 6 min) to give Compound 2 as a white solid. MS mass calculated for [M+H]+ (C33H35N7O3) requires m/z 578.3, LCMS found m/z 578.3. 1H NMR (400 MHz, MeOD-d4) δ 8.34 (d, J=1.2 Hz, 1H), 7.98 (dd, J=1.6, 8.6 Hz, 1H), 7.67 (d, J=8.4 Hz, 1H), 7.48 (s, 1H), 7.44 (dd, J=1.8, 7.8 Hz, 1H), 7.39-7.32 (m, 1H), 7.29 (d, J=7.8 Hz, 1H), 6.15 (d, J=8.2 Hz, 1H), 6.04 (d, J=8.0 Hz, 1H), 5.33-5.23 (m, 1H), 4.94-4.89 (m, 1H), 4.79-4.71 (m, 1H), 4.69-4.57 (m, 1H), 4.47 (td, J=6.0, 9.0 Hz, 1H), 4.11-3.90 (m, 2H), 3.82 (br d, J=3.4 Hz, 4H), 3.59-3.47 (m, 4H), 3.08-2.96 (m, 4H), 2.87-2.74 (m, 1H), 2.71-2.59 (m, 4H), 2.58-2.48 (m, 1H).

### Example 3 (General Procedure C)

### (S)-2-((4-(6-((1-methyl-1H-benzo[d]imidazol-5-yl)methoxy)pyridin-2-yl)piperazin-1-yl)methyl)-1-(oxetan-2-ylmethyl)-1H-benzo[d]imidazole-6-carboxylic acid

The title compound was prepared according to Scheme 2. This General Procedure C exemplifies Scheme 2 and provides particular synthetic details as applied to the title compound.

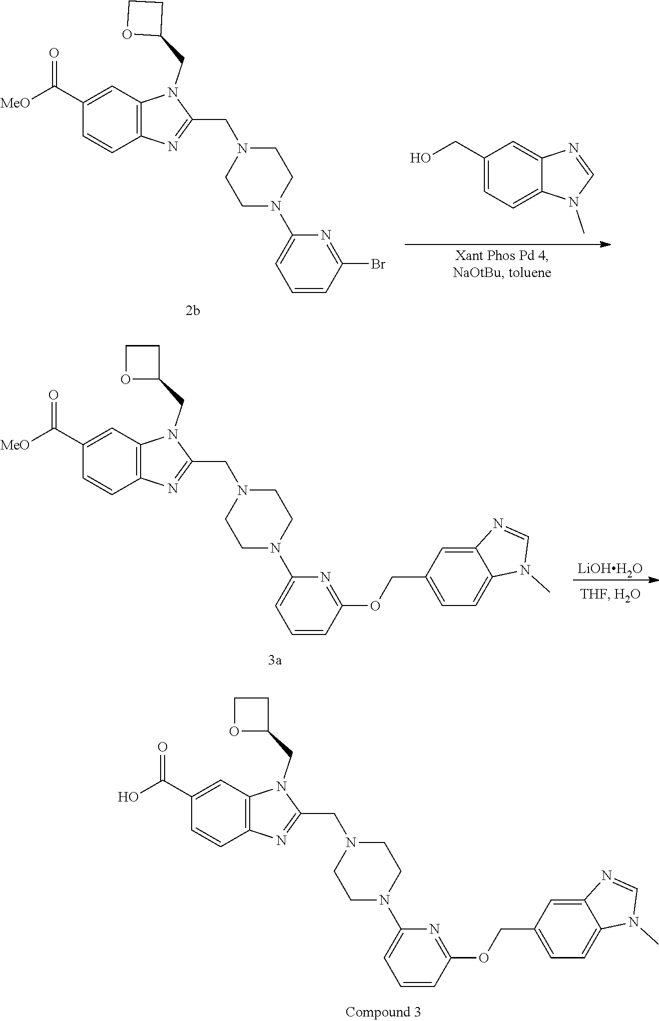

### (S)-methyl 2-((4-(6-((1-methyl-1H-benzo[d]imidazol-5-yl)methoxy)pyridin-2-yl)piperazin-1-yl)methyl)-1-(oxetan-2-ylmethyl)-1H-benzo[d]imidazole-6-carboxylate (3a)

t-BuONa (57.62 mg, 599.54 umol, 3 eq) and (5-diphenylphosphanyl-9,9-dimethyl-xanthen-4-yl)-diphenyl-phosphane; methanesulfonate; XantPhos Pd G4 (19.23 mg, 19.98 umol, 0.1 eq) was added to a solution of (S)-methyl 2-((4-(6-bromopyridin-2-yl)piperazin-1-yl)methyl)-1-(oxetan-2-ylmethyl)-1H-benzo[d]imidazole-6-carboxylate (2b, 100 mg, 199.85 umol, 1 eq) and (1-methyl-1H-benzo[d]imidazol-5-yl)methanol (38.90 mg, 239.81 umol, 1.2 eq) in toluene (10 mL) at 20° C. under N2. The mixture was stirred at 100° C. for 16 hours under N2. LCMS showed the reaction was completed. The mixture was filtered, and the filtrate concentrated to give 3a as a gray solid.

### (S)-2-((4-(6-((1-methyl-1H-benzo[d]imidazol-5-yl)methoxy)pyridin-2-yl)piperazin-1-yl)methyl)-1-(oxetan-2-ylmethyl)-1H-benzo[d]imidazole-6-carboxylic acid (Compound 3)

LiOH·H2O (23.81 mg, 567.34 umol, 3 eq) was added to the solution of (S)-methyl 2-((4-(6-((1-methyl-1H-benzo[d]imidazol-5-yl)methoxy)pyridin-2-yl)piperazin-1-yl)methyl)-1-(oxetan-2-ylmethyl)-1H-benzo[d]imidazole-6-carboxylate (3a, 110 mg, 189.11 umol, 1 eq) in THF (7 mL) and H2O (3 mL) at 20° C. Then the solution was stirred at 20° C. for 16 hours. LCMS showed 3a was consumed, and desired MS was detected. The mixture was adjusted to pH=7 with CH3COOH. The mixture was extracted with Ethyl acetate (10 mL*6). The combined organic layers were washed with brine (15 mL), dried over Na2SO4, filtered and concentrated in vacuo. The residue was purified by prep-HPLC (column: Phenomenex Gemini-NX C18 75*30 mm*3 um; mobile phase: [water (10 mM NH4HCO3)-ACN]; B %: 25%-45%, 8 min) to give Compound 3 as white solid. MS mass calculated for [M+H]+ (C31H33N7O4) requires m/z 568.3, LCMS found m/z 568.3. 1H NMR (400 MHz, MeOD-d4) δ 8.32 (br s, 1H), 8.09 (br s, 1H), 7.97 (br d, J=8.4 Hz, 1H), 7.75-7.59 (m, 2H), 7.57-7.37 (m, 3H), 6.25 (br d, J=7.6 Hz, 1H), 6.11 (br d, J=7.4 Hz, 1H), 5.42 (s, 2H), 5.27 (br s, 1H), 4.96-4.91 (m, 1H), 4.75 (br s, 1H), 4.63 (br d, J=6.4 Hz, 1H), 4.46 (br d, J=5.0 Hz, 1H), 4.08-3.97 (m, 1H), 3.95-3.83 (m, 4H), 3.52 (br s, 4H), 2.85-2.71 (m, 1H), 2.68-2.44 (m, 5H).

### Example 4 (General Procedure D)

### 2-((4-(6-(6-cyano-8-fluoro-1,2,3,4-tetrahydronaphthalen-2-yl)pyridin-2-yl)piperazin-1-yl)methyl)-1-((S)-oxetan-2-ylmethyl)-1H-benzo[d]imidazole-6-carboxylic acid

The title compound was prepared according to Scheme 2. This General Procedure D exemplifies Scheme 2 and provides particular synthetic details as applied to the title compound.

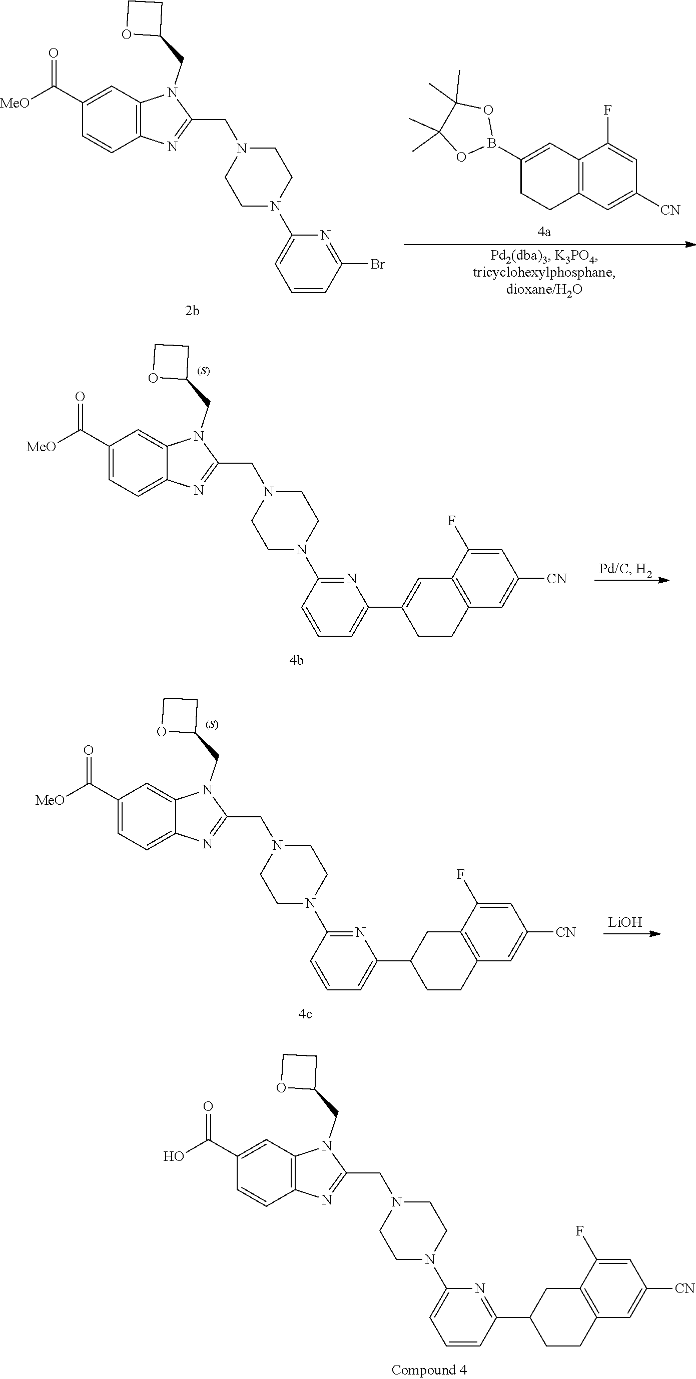

### (S)-methyl 2-((4-(6-(6-cyano-8-fluoro-3,4-dihydronaphthalen-2-yl)pyridin-2-yl)piperazin-1-yl)methyl)-1-(oxetan-2-ylmethyl)-1H-benzo[d]imidazole-6-carboxylate (4b)

To a mixture of (S)-methyl 2-((4-(6-bromopyridin-2-yl)piperazin-1-yl)methyl)-1-(oxetan-2-ylmethyl)-1H-benzo[d]imidazole-6-carboxylate (2b, 500 mg, 999.23 umol, 1 eq), 4-fluoro-6-(4,4,5,5-tetramethyl-1,3,2-dioxaborolan-2-yl)-7,8-dihydronaphthalene-2-carbonitrile (4a, 551.84 mg, 1.20 mmol, 65% purity, 1.2 eq) in dioxane (20 mL) and H2O (2 mL) was added Pd2(dba)3 (91.50 mg, 99.92 umol, 0.1 eq), tricyclohexylphosphane (56.04 mg, 199.85 umol, 64.79 uL, 0.2 eq) and K3PO4 (530.25 mg, 2.50 mmol, 2.5 eq) under N2. The mixture was degassed and purged with N2 3 times, and then the mixture was stirred at 120° C. for 16 hours under N2. LCMS showed 2n was consumed, and desired mass was detected. The reaction mixture was concentrated under reduced pressure. The residue was purified by column chromatography (SiO2, Petroleum ether:Ethyl acetate=5:1 to 0:1) to give 4b as a white solid. 1H NMR (400 MHz, CDCl3-d) δ 8.18 (d, J=1.0 Hz, 1H), 8.08-7.93 (m, 1H), 7.77 (d, J=8.6 Hz, 1H), 7.65-7.44 (m, 2H), 7.25 (s, 1H), 7.20 (d, J=9.2 Hz, 1H), 7.01 (d, J=7.6 Hz, 1H), 6.63 (d, J=8.4 Hz, 1H), 5.33-5.18 (m, 1H), 4.88-4.57 (m, 3H), 4.49-4.34 (m, 1H), 4.08-4.00 (m, 2H), 3.96 (s, 3H), 3.76-3.50 (m, 4H), 3.06-2.83 (m, 4H), 2.81-2.64 (m, 5H), 2.58-2.39 (m, 1H).

### Methyl 2-((4-(6-(6-cyano-8-fluoro-1,2,3,4-tetrahydronaphthalen-2-yl)pyridin-2-yl)piperazin-1-yl)methyl)-1-((S)-oxetan-2-ylmethyl)-1H-benzo[d]imidazole-6-carboxylate (4c)

To a solution of (S)-methyl 2-((4-(6-(6-cyano-8-fluoro-3,4-dihydronaphthalen-2-yl)pyridin-2-yl)piperazin-1-yl)methyl)-1-(oxetan-2-ylmethyl)-1H-benzo[d]imidazole-6-carboxylate (4b, 150 mg, 253.10 umol, 1 eq) in THF (4 mL) was added Pd/C (150.00 mg, 141.92 umol, 10% purity, 0.5 eq). The mixture was stirred at 40° C. for 5 hours under H2 (50 psi). LCMS showed 4b was consumed, and desired mass was detected. The reaction mixture was filtered and the filtrate was concentrated under reduced pressure. The residue was purified by prep-TLC (SiO2, EtOAc:MeOH=20:1) to give 4c as a white solid. The product was used directly in next step without any further purification.

### 2-((4-(6-(6-cyano-8-fluoro-1,2,3,4-tetrahydronaphthalen-2-yl)pyridin-2-yl)piperazin-1-yl)methyl)-1-((S)-oxetan-2-ylmethyl)-1H-benzo[d]imidazole-6-carboxylic acid (Compound 4)

To a solution of methyl 2-((4-(6-(6-cyano-8-fluoro-1,2,3,4-tetrahydronaphthalen-2-yl)pyridin-2-yl)piperazin-1-yl)methyl)-1-((S)-oxetan-2-ylmethyl)-1H-benzo[d]imidazole-6-carboxylate (4c, 100 mg, 168.16 umol, 1 eq) in THF (7 mL) and H2O (3 mL) was added LiOH·H2O (10.58 mg, 252.24 umol, 1.5 eq). The mixture was stirred at 15° C. for 24 hours. LCMS showed 4c was consumed, and desired mass was detected. Citric acid (aq. 1 M) was added to the reaction mixture until pH=5-6, and then the mixture was filtered to collect solid. The filter cake was purified by prep-HPLC (neutral condition; column: mobile phase: [water (10 mM NH4HCO3)-ACN]; B %: 25%-55%, 10 min) to give Compound 4 as a white solid. MS mass calculated for [M+H]+ (C33H33FN6O3) requires m/z 581.3, LCMS found m/z 581.3. 1H NMR (400 MHz, MeOD-d4) δ 8.33 (s, 1H), 7.97 (dd, J=1.4, 8.4 Hz, 1H), 7.67 (d, J=8.4 Hz, 1H), 7.54-7.39 (m, 1H), 7.32 (s, 1H), 7.26 (d, J=9.0 Hz, 1H), 6.63 (s, 1H), 6.61 (d, J=2.0 Hz, 1H), 5.35-5.20 (m, 1H), 4.91 (br d, J=7.2 Hz, 1H), 4.76-4.69 (m, 1H), 4.68-4.59 (m, 1H), 4.47 (td, J=6.0, 9.0 Hz, 1H), 4.10-3.84 (m, 2H), 3.50 (br s, 4H), 3.13-2.99 (m, 3H), 2.90 (br d, J=5.4 Hz, 2H), 2.85-2.73 (m, 1H), 2.60 (br s, 4H), 2.56-2.47 (m, 1H), 2.20-2.07 (m, 1H), 2.06-1.89 (m, 1H).

### Example 5 (General Procedure E)

### (S)-2-((4-(6-(5-cyano-3,4-dihydroquinolin-1(2H)-yl)pyridin-2-yl)piperazin-1-yl)methyl)-1-(oxetan-2-ylmethyl)-1H-benzo[d]imidazole-6-carboxylic acid

The title compound was prepared according to Scheme 3. This General Procedure C exemplifies Scheme 3 and provides particular synthetic details as applied to the title compound.

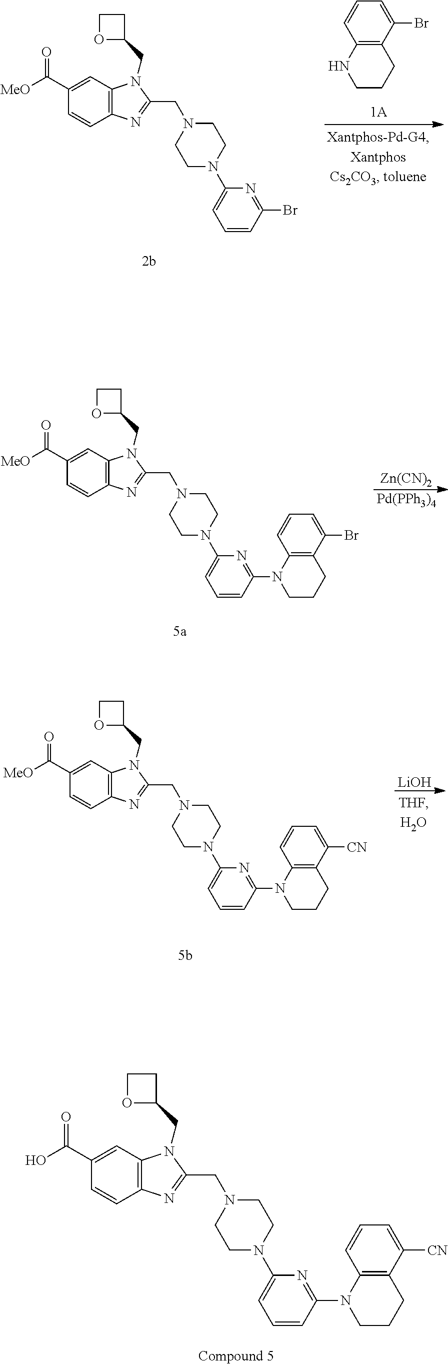

### (S)-methyl 2-((4-(6-(5-bromo-3,4-dihydroquinolin-1(2H)-yl)pyridin-2-yl)piperazin-1-yl)methyl)-1-(oxetan-2-ylmethyl)-1H-benzo[d]imidazole-6-carboxylate (5a)

To a mixture of (S)-methyl 2-((4-(6-bromopyridin-2-yl)piperazin-1-yl)methyl)-1-(oxetan-2-ylmethyl)-1H-benzo[d]imidazole-6-carboxylate (2b, 230 mg, 459.64 umol, 1 eq) and 5-bromo-1,2,3,4-tetrahydroquinoline (1a, 116.98 mg, 551.57 umol, 1.2 eq) in toluene (12 mL) was added Cs2CO3 (748.80 mg, 2.30 mmol, 5 eq), Xantphos (39.89 mg, 68.95 umol, 0.15 eq) and XantPhos Pd G4 (41.67 mg, 45.96 umol, 0.1 eq) under N2. The mixture was stirred at 120° C. for 16 hours under N2. LCMS showed 2b was consumed completely and desired mass was detected. The mixture was diluted with water (20 mL) and extracted with ethyl acetate (40 mL*3). The combined organic layers were washed with brine (30 mL), dried over with anhydrous Na2SO4, filtered and concentrated in vacuo. The residue was purified by prep-HPLC (column: Waters Xbridge BEH C18 100*30 mm*10 um; mobile phase: [water (10 mM NH4HCO3)-ACN]; B %: 50%-80%, 8 min) to give 5a as an off-white solid. 1H NMR (400 MHz, CDCl3-d) δ 8.18 (d, J=1.0 Hz, 1H), 7.99 (dd, J=1.6, 8.4 Hz, 1H), 7.77 (d, J=8.4 Hz, 1H), 7.33-7.28 (m, 2H), 7.16 (d, J=7.0 Hz, 1H), 6.90 (t, J=8.0 Hz, 1H), 6.42 (d, J=8.0 Hz, 1H), 6.14 (d, J=8.0 Hz, 1H), 5.28-5.21 (m, 1H), 4.75 (br s, 1H), 4.79-4.69 (m, 1H), 4.69-4.61 (m, 1H), 4.45-4.35 (m, 1H), 4.01 (s, 2H), 3.96 (s, 3H), 3.91-3.83 (m, 2H), 3.59-3.46 (m, 4H), 2.82 (t, J=6.8 Hz, 2H), 2.78-2.72 (m, 1H), 2.66 (br t, J=4.8 Hz, 4H), 2.52-2.42 (m, 1H), 1.99-1.89 (m, 2H).

### (S)-methyl 2-((4-(6-(5-cyano-3,4-dihydroquinolin-1(2H)-yl)pyridin-2-yl)piperazin-1-yl)methyl)-1-(oxetan-2-ylmethyl)-1H-benzo[d]imidazole-6-carboxylate (5b)

To a mixture of (S)-methyl 2-((4-(6-(5-bromo-3,4-dihydroquinolin-1(2H)-yl)pyridin-2-yl)piperazin-1-yl)methyl)-1-(oxetan-2-ylmethyl)-1H-benzo[d]imidazole-6-carboxylate (5a, 60 mg, 95.00 umol, 1 eq) in DMA (3 mL) were added Zn(CN)2 (44.62 mg, 380.01 umol, 24.12 uL, 4 eq) and Pd(PPh3)4 (10.98 mg, 9.50 umol, 0.1 eq) under N2. The mixture was stirred at 160° C. for 0.5 hours. LCMS showed 5a was consumed completely and desired mass was detected. The reaction mixture was filtered and the filtrate was poured into water (20 mL). The aqueous phase was extracted with ethyl acetate (40 mL*2). The combined organic layers were washed with brine (30 mL*2), dried over with anhydrous Na2SO4, filtered and concentrated in vacuo. The residue was purified by prep-TLC (SiO2, ethyl acetate:MeOH=10:1) to give 5b as a light yellow solid. 1H NMR (400 MHz, CDCl3-d) δ 8.17 (d, J=1.0 Hz, 1H), 7.99 (dd, J=1.6, 8.4 Hz, 1H), 7.77 (d, J=8.4 Hz, 1H), 7.39-7.34 (m, 1H), 7.33 (br s, 1H), 7.18 (dd, J=1.0, 7.6 Hz, 1H), 7.08 (t, J=8.0 Hz, 1H), 6.40 (d, J=8.0 Hz, 1H), 6.20 (d, J=8.2 Hz, 1H), 5.28-5.21 (m, 1H), 4.77-4.61 (m, 3H), 4.40 (td, J=5.8, 9.0 Hz, 1H), 4.05-3.99 (m, 2H), 3.96 (s, 3H), 3.89-3.83 (m, 2H), 3.51 (br d, J=2.8 Hz, 4H), 3.03-2.94 (m, 2H), 2.84-2.70 (m, 1H), 2.65 (t, J=5.0 Hz, 4H), 2.46 (tdd, J=7.2, 9.0, 11.3 Hz, 1H), 2.15-1.98 (m, 2H).

### (S)-2-((4-(6-(5-cyano-3,4-dihydroquinolin-1(2H)-yl)pyridin-2-yl)piperazin-1-yl)methyl)-1-(oxetan-2-ylmethyl)-1H-benzo[d]imidazole-6-carboxylic acid (Compound 5)

To a mixture of (S)-methyl 2-((4-(6-(5-cyano-3,4-dihydroquinolin-1(2H)-yl)pyridin-2-yl)piperazin-1-yl)methyl)-1-(oxetan-2-ylmethyl)-1H-benzo[d]imidazole-6-carboxylate (5b, 78 mg, 135.02 umol, 1 eq) in THF (2.8 mL) was added a solution of LiOH·H2O (8.50 mg, 202.54 umol, 1.5 eq) in H2O (1.2 mL) under N2. The mixture was stirred at 20° C. for 32 hours. LCMS showed 5b was consumed completely and desired mass was detected. Citric acid (aq. 10%) was added to the reaction mixture until pH=7, and the mixture was concentrated under reduced pressure. The residue was purified by prep-HPLC (column: Waters Xbridge Prep OBD C18 150*40 mm*10 um; mobile phase: [water (10 mM NH4HCO3)-ACN]; B %: 20%-50%, 8 min) to give Compound 5 as a white solid. MS mass calculated for [M+H]+ (C32H33N7O3) requires m/z 564.3, LCMS found m/z 564.3. 1H NMR (400 MHz, CDCl3-d) δ 8.24 (s, 1H), 8.06 (d, J=8.6 Hz, 1H), 7.82 (d, J=8.6 Hz, 1H), 7.57 (d, J=8.6 Hz, 1H), 7.37 (t, J=8.0 Hz, 1H), 7.18 (d, J=7.6 Hz, 1H), 7.08 (t, J=8.0 Hz, 1H), 6.40 (d, J=7.8 Hz, 1H), 6.20 (d, J=8.2 Hz, 1H), 5.26 (br s, 1H), 4.81-4.62 (m, 3H), 4.42 (td, J=6.0, 9.0 Hz, 1H), 4.04 (s, 2H), 3.89-3.83 (m, 2H), 3.53 (br s, 4H), 2.99 (t, J=6.6 Hz, 2H), 2.81-2.73 (m, 1H), 2.67 (br s, 4H), 2.58-2.38 (m, 1H), 2.02 (quin, J=6.2 Hz, 2H).

### Example 6 (General Procedure F)

### 3-(4-(6-((4-cyano-2-fluorobenzyl)oxy)pyridin-2-yl)piperidin-1-yl)-2,3-dihydro-11H-benzo[d]pyrrolo[1,2-a]imidazole-7-carboxylic acid

The title compound was prepared according to Scheme 4. This General Procedure D exemplifies Scheme 4 and provides particular synthetic details as applied to the title compound.

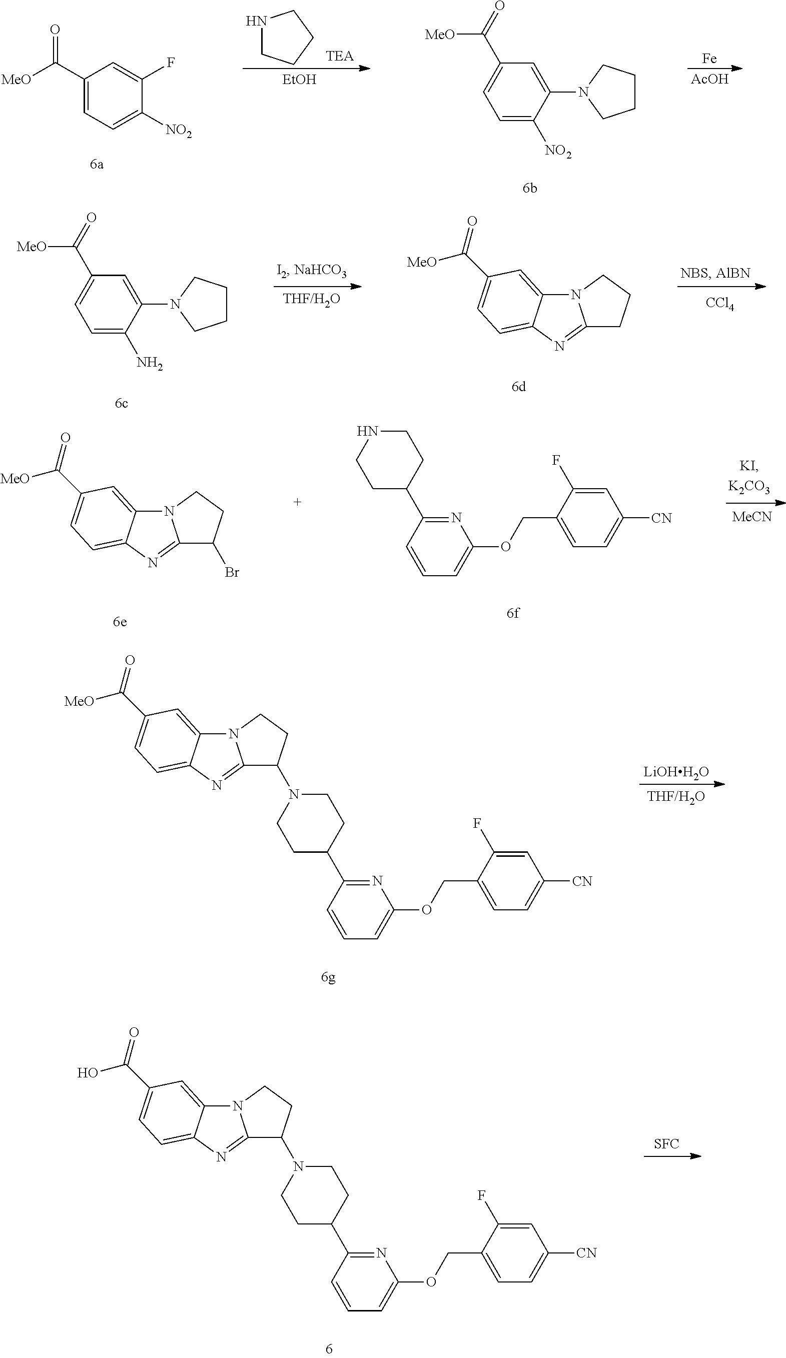

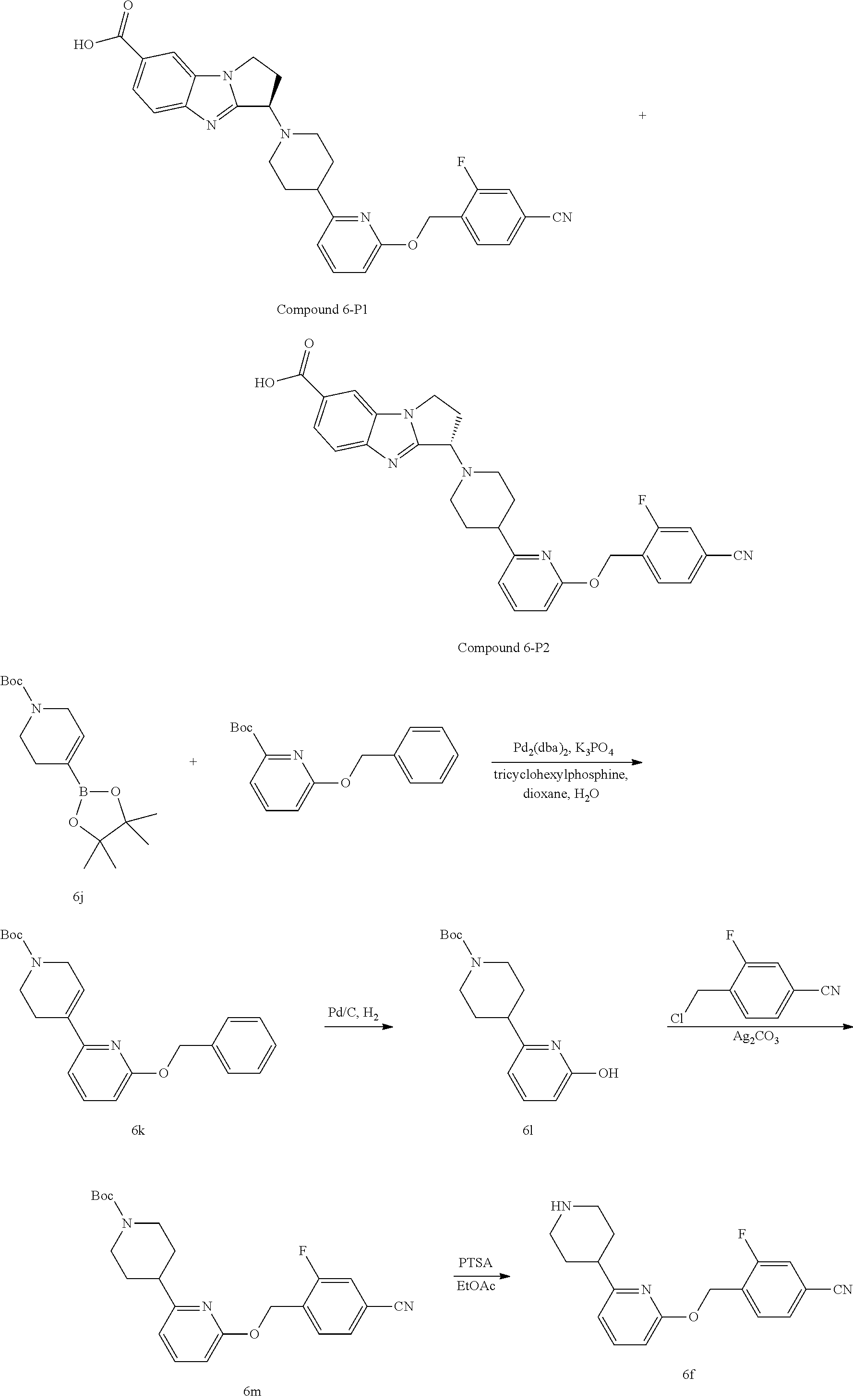

Methyl 4-nitro-3-(pyrrolidin-1-yl)benzoate (6b). TEA (1.14 g, 11.30 mmol, 1.57 mL, 3 eq) was added to the solution of methyl 3-fluoro-4-nitrobenzoate (6a, 750 mg, 3.77 mmol, 1 eq) and pyrrolidine (321.43 mg, 4.52 mmol, 377.27 uL, 1.2 eq) in EtOH (10 mL) at 0° C. Then the solution was stirred at 35° C. for 3 hours. TLC (Petroleum ether:Ethyl acetate=10:1) showed 6a was consumed and one new major spot was formed. The mixture was concentrated to remove the solvent. The residue was triturated with H2O (30 mL) and filtered. The solid was dried over in vacuo to give 6a as a yellow solid. The product was used in next step without further purification. 1H NMR (400 MHz, MeOD-d4) δ 7.73 (d, J=8.4 Hz, 1H), 7.64 (d, J=1.6 Hz, 1H), 7.31 (dd, J=1.6, 8.4 Hz, 1H), 3.92 (s, 3H), 3.27-3.21 (m, 4H), 2.05-1.98 (m, 4H).

Methyl 4-amino-3-(pyrrolidin-1-yl)benzoate (6c). Fe (3.79 g, 67.93 mmol, 10 eq) was added to the solution of methyl 4-nitro-3-(pyrrolidin-1-yl)benzoate (6b, 1.7 g, 6.79 mmol, 1 eq) in CH3COOH (20 mL) at 20° C. Then the solution was stirred at 35° C. for 1 hour. TLC (Petroleum ether:Ethyl acetate=10:1) showed 6b was consumed and one new major spot was formed. The mixture was filtered and the filtrate was extracted with ethyl acetate (30 mL*3). The combined organic layers were washed with saturated Na2CO3 (40 mL), brine (60 mL), dried over Na2SO4, filtered and concentrated in vacuo to give 6c as yellow oil. The product was used to next step directly. 1H NMR (400 MHz, MeOD-d4) δ 7.61 (d, J=1.8 Hz, 1H), 7.52 (dd, J=2.0, 8.4 Hz, 1H), 6.71 (d, J=8.4 Hz, 1H), 3.82 (s, 3H), 3.06-2.98 (m, 4H), 1.98-1.91 (m, 4H).

### Methyl 2,3-dihydro-1H-benzo[d]pyrrolo[1,2-a]imidazole-7-carboxylate (6d)

To a solution of methyl 4-amino-3-(pyrrolidin-1-yl)benzoate (6c, 900 mg, 4.09 mmol, 1 eq) in THF (48 mL) and H2O (16 mL) was added NaHCO₃ (3.43 g, 40.86 mmol, 1.59 mL, 10 eq) and 12 (7.78 g, 30.64 mmol, 6.17 mL, 7.5 eq) at 20° C. Then the solution was stirred at 20° C. for 3 hours. TLC (Petroleum ethe:Ethyl acetate=10:1) showed 6c was consumed and one new major spot was formed. The solution was quenched with saturated Na2S203 (100 mL), and extracted with ethyl acetate (30 mL*3). The combined organic layers were washed with brine (50 mL), dried over Na2SO4, filtered and concentrated. The crude product was used for next step without further purification. 6d was obtained as a brown solid. 1H NMR (400 MHz, CDCl3-d) δ 8.11 (d, J=1.3 Hz, 1H), 7.91 (dd, J=1.6, 8.6 Hz, 1H), 7.60 (d, J=8.6 Hz, 1H), 4.23 (t, J=7.2 Hz, 2H), 3.93 (s, 3H), 3.12-3.06 (m, 2H), 2.78 (t, J=7.2 Hz, 2H).

### Methyl 3-bromo-2,3-dihydro-1H-benzo[d]pyrrolo[1,2-a]imidazole-7-carboxylate (6e)

To a solution of methyl 2,3-dihydro-1H-benzo[d]pyrrolo[1,2-a]imidazole-7-carboxylate (6d, 1.1 g, 5.09 mmol, 1 eq) in CCl4 (40 mL) was added AIBN (417.67 mg, 2.54 mmol, 0.5 eq) and NBS (995.93 mg, 5.60 mmol, 1.1 eq) at 20° C. Then the solution was stirred at 85° C. for 3 hours. Then the solution was stirred at 85° C. for 1 hour. TLC (Petroleum ether:Ethyl acetate=1:1) showed trace of 6d was remained and one nes spot was formed. The mixture was concentrated to remove the solvent and extracted with Ethyl acetate (20 mL*3). The combined organic layers were washed with brine (25 mL), dried over Na2SO4, filtered and concentrated under reduced pressure. The residue was purified by column chromatography (SiO2, Petroleum ether:Ethyl acetate=50:1 to 10:1) to give 6e as a brown solid. 1H NMR (400 MHz, MeOD-d4) δ 8.26 (s, 1H), 7.99 (dd, J=1.4, 8.8 Hz, 1H), 7.70 (d, J=8.8 Hz, 1H), 5.60 (dd, J=1.6, 6.8 Hz, 1H), 4.42-4.30 (m, 2H), 3.94 (s, 3H), 3.50-3.35 (m, 1H), 3.05-2.96 (m, 1H).

Methyl 3-(4-(6-((4-cyano-2-fluorobenzyl)oxy)pyridin-2-yl)piperidin-1-yl)-2,3-dihydro-1H-benzo[d]pyrrolo[1,2-a]imidazole-7-carboxylate (6g). KI (227.80 mg, 1.37 mmol, 1.5 eq) was added to the solution of methyl 3-bromo-2,3-dihydro-1H-benzo[d]pyrrolo[1,2-a]imidazole-7-carboxylate (6e, 270 mg, 914.85 umol, 1 eq) in CH3CN (20 mL) at 20° C. The mixture was stirred at 20° C. for 0.5 hours. Then 3-fluoro-4-(((6-(piperidin-4-yl)pyridin-2-yl)oxy)methyl)benzonitrile (6f, 341.81 mg, 1.10 mmol, 1.2 eq) and K2CO3 (189.66 mg, 1.37 mmol, 1.5 eq) was added to the solution at 20° C. The reaction was stirred at 80° C. for 3 hours. TLC (Petroleum ether:Ethyl acetate=0:1) showed 6e was consumed and one new major spot was formed. The solution was concentrated to remove the solvent. The mixture was extracted with ethyl acetate (5 mL*3). The combined organic layers were washed with brine (15 mL), dried over Na2SO4, filtered and concentrated under reduced pressure. The residue was purified by column chromatography (SiO2, Petroleum ether:Ethyl acetate=10:1 to 0:1) to give 6g as a brown solid. 1H NMR (400 MHz, MeOD-d4) δ 8.21 (d, J=1.6 Hz, 1H), 7.96 (dd, J=1.6, 8.6 Hz, 1H), 7.73-7.61 (m, 2H), 7.60-7.49 (m, 3H), 6.83 (d, J=7.4 Hz, 1H), 6.68 (d, J=8.4 Hz, 1H), 5.50 (s, 2H), 4.42 (br d, J=4.0 Hz, 3H), 4.26-4.16 (m, 1H), 3.94 (s, 3H), 3.11-2.99 (m, 1H), 3.22-2.98 (m, 1H), 2.94-2.84 (m, 1H), 2.82-2.71 (m, 1H), 2.68-2.57 (m, 1H), 2.18 (br dd, J=11.0, 14.8 Hz, 1H), 1.95-1.82 (m, 4H).

### 3-(4-(6-((4-cyano-2-fluorobenzyl)oxy)pyridin-2-yl)piperidin-1-yl)-2,3-dihydro-1H-benzo[d]pyrrolo[1,2-a]imidazole-7-carboxylic acid (6)

To a solution of methyl 3-(4-(6-((4-cyano-2-fluorobenzyl)oxy)pyridin-2-yl)piperidin-1-yl)-2,3-dihydro-1H-benzo[d]pyrrolo[1,2-a]imidazole-7-carboxylate (6g, 320 mg, 608.86 umol, 1 eq) in THF (22.4 mL) and H2O (9.6 mL) was added LiOH·H2O (25.55 mg, 608.86 umol, 1 eq) at 20° C. LCMS showed 6g was remained, and desired mass was detected. LiOH·H2O (25.55 mg, 608.86 umol, 1 eq) was added to the solution at 20° C. Then the reaction was stirred at 20° C. for another 24 hours. LCMS detected the desired product ass and showed that the 6g was consumed. The mixture was adjusted to pH=5 with HCl (1 M, 10 mL) and filtered. The filtrate was concentrated under reduced pressure. The residue was purified by prep-HPLC (Phenomenex Luna C18 200*40 mm*10 um; mobile phase: [water (0.2% FA)-ACN]; B %: 35%-65%, 10 min) to give Compound 6 as a white solid. 1H NMR (400 MHz, MeOD-d4) δ 8.14-8.10 (m, 1H), 7.89 (d, J=10.4 Hz, 1H), 7.81 (dd, J=1.6, 8.6 Hz, 1H), 7.74-7.60 (m, 4H), 6.88 (d, J=7.2 Hz, 1H), 6.71 (d, J=8.2 Hz, 1H), 5.46 (s, 2H), 4.35-4.23 (m, 2H), 4.19-4.09 (m, 1H), 3.28-3.18 (m, 2H), 2.96-2.80 (m, 2H), 2.77-2.62 (m, 1H), 2.76-2.57 (m, 2H), 2.10-1.98 (m, 1H), 1.83-1.64 (m, 4H).

(R)-3-(4-(6-((4-cyano-2-fluorobenzyl)oxy)pyridin-2-yl)piperidin-1-yl)-2,3-dihydro-1H-benzo[d]pyrrolo[1,2-a]imidazole-7-carboxylic acid and (S)-3-(4-(6-((4-cyano-2-fluorobenzyl)oxy)pyridin-2-yl)piperidin-1-yl)-2,3-dihydro-1H-benzo[d]pyrrolo[1,2-a]imidazole-7-carboxylic acid (Compounds 6-P1 & 6-P2). 3-(4-(6-((4-cyano-2-fluorobenzyl)oxy)pyridin-2-yl)piperidin-1-yl)-2,3-dihydro-1H-benzo[d]pyrrolo[1,2-a]imidazole-7-carboxylic acid (6, 150 mg, 293.23 umol, 1 eq) was separated by Chiral SFC to give Compound 6-P1 as white solid. MS mass calculated for [M+1]+(C29H26FN5O3) requires m/z 512.2, LCMS found m/z 512.3; 1H NMR (400 MHz, MeOD-d4) δ 8.19 (s, 1H), 7.97 (br d, J=8.4 Hz, 1H), 7.76-7.46 (m, 5H), 6.84 (br d, J=7.4 Hz, 1H), 6.68 (d, J=8.2 Hz, 1H), 5.51 (s, 2H), 4.48-4.13 (m, 3H), 3.16-2.98 (m, 2H), 2.98-2.86 (m, 1H), 2.78 (br dd, J=8.6, 13.6 Hz, 1H), 2.71-2.56 (m, 1H), 2.29-2.16 (m, 1H), 1.96-1.76 (m, 4H).

Compound 6-P2 was obtained as white solid. MS mass calculated for [M+1]+(C29H26FN5O3) requires m/z 512.2, LCMS found m/z 512.3; 1H NMR (400 MHz, MeOD-d4) δ 8.24 (s, 1H), 7.99 (br d, J=8.7 Hz, 1H), 7.78-7.50 (m, 5H), 6.88 (br d, J=7.4 Hz, 1H), 6.72 (d, J=8.2 Hz, 1H), 5.49 (br s, 2H), 4.81-4.73 (m, 1H), 4.49-4.21 (m, 2H), 3.74 (br d, J=10.5 Hz, 1H), 3.43 (br d, J=10.6 Hz, 1H), 3.19 (br dd, J=5.4, 14.0 Hz, 1H), 2.99-2.73 (m, 2H), 2.66 (br s, 1H), 2.11-1.94 (m, 4H), 0.10-0.10 (m, 1H).

When a mixture of stereoisomers is separated by HPLC, it is to be appreciated that the resultant individual stereoisomers or mixtures will be assigned sequential labels (e.g., P1, P2, etc.), the order of which implies the order in which the isomers eluted from the HPLC column. In the examples described herein, when the mixture of stereoisomers is separated by HPLC, it is to be appreciated that the first-eluting mixture of disasteromers is labeled “P1,” and the second-eluting mixture of disasteromers is labeled “P2.” The absolute configuration of compounds, e.g., Compounds 6-P1 & 6-P2 may be obtained by known methods.

### Tert-butyl 6-(benzyloxy)-5′,6′-dihydro-[2,4′-bipyridine]-1′(2′H)-carboxylate (6k)

To a mixture of tert-butyl 4-(4,4,5,5-tetramethyl-1,3,2-dioxaborolan-2-yl)-5,6-dihydropyridine-1(2H)-carboxylate (6j, 10 g, 32.34 mmol, 1 eq) and 2-(benzyloxy)-6-bromopyridine (8.54 g, 32.34 mmol, 1 eq) in dioxane (100 mL) and H2O (10 mL) was added K3PO4 (17.16 g, 80.85 mmol, 2.5 eq), Pd2(dba)3 (1.48 g, 1.62 mmol, 0.05 eq) and tricyclohexylphosphine (906.92 mg, 3.23 mmol, 1.05 mL, 0.1 eq). The resulted reaction mixture was stirred at 100° C. for 16 hours under N2. LCMS showed one major peak with desired MS was detected. The reaction mixture was poured into water (300 mL) and extracted with EtOAc (300 mL*2). The combined organic layer was concentrated in vacuo. The residue was purified by silica gel column chromatography (petroleum ether:ethyl acetate=10:1) to give crude 6k as yellow oil. The crude product was used in the next step without purification. 1H NMR (400 MHz, MeOD-d4) δ 7.56 (t, J=7.8 Hz, 1H) 7.47 (d, J=7.6 Hz, 2H) 7.38 (t, J=7.2 Hz, 2H) 7.29-7.35 (m, 1H) 6.95 (d, J=7.6 Hz, 1H) 6.73 (br s, 1H) 6.69 (d, J=8.2 Hz, 1H) 5.42 (s, 2H) 4.11-4.19 (m, 2H) 3.66 (brt, J=5.2 Hz, 2H) 2.62 (br s, 2 H) 1.50 (s, 9H).

### Tert-butyl 4-(6-hydroxypyridin-2-yl)piperidine-1-carboxylate (6l)

To a solution of tert-butyl 6-(benzyloxy)-5′,6′-dihydro-[2,4′-bipyridine]-1′(2′H)-carboxylate (6k, 3.5 g, 9.55 mmol, 1 eq) in MeOH (30 mL) was added Pd/C (0.3 g, 10% purity). The resulted reaction mixture was stirred at 20° C. under H2 (15 Psi) for 5 hours. LCMS showed 6k was consumed, and desired mass was detected. The reaction mixture was filtered and the filtrate was concentrated in vacuo. The residue was triturated with MTBE (30 mL) and filtered. The solid was dried in vacuo to give 61 as a white solid. 1H NMR (400 MHz, MeOD-d4) δ 11.86 (br s, 1H) 7.40 (dd, J=9.0, 7.0 Hz, 1H) 6.43 (d, J=9.0 Hz, 1H) 6.05 (d, J=7.0 Hz, 1H) 4.26 (br s, 2H) 2.86 (br s, 2H) 2.66 (br t, J=12.2 Hz, 1H) 1.95 (br d, J=12.8 Hz, 2H) 1.56-1.67 (m, 4H) 1.50 (s, 9H).

### Tert-butyl 4-(6-((4-cyano-2-fluorobenzyl)oxy)pyridin-2-yl)piperidine-1-carboxylate (6m)

To a mixture of tert-butyl 4-(6-hydroxypyridin-2-yl)piperidine-1-carboxylate (61, 6.5 g, 23.35 mmol, 1 eq) and 4-(chloromethyl)-3-fluorobenzonitrile (5.50 g, 25.69 mmol, 1.1 eq) in toluene (100 mL) was added Ag2CO3 (12.88 g, 46.70 mmol, 2.12 mL, 2 eq). The reaction mixture was stirred at 80° C. for 16 hours. LCMS showed 61 was consumed, and desired mass was detected. The reaction mixture was cooled to room temperature and filtered. The filtrate was concentrated to give 6m as a light yellow oil. The product was used in the next step without purification.

### 3-fluoro-4-(((6-(piperidin-4-yl)pyridin-2-yl)oxy)methyl)benzonitrile (6f)

To a solution of tert-butyl 4-(6-((4-cyano-2-fluorobenzyl)oxy)pyridin-2-yl)piperidine-1-carboxylate (6m, 9.5 g, 23.09 mmol, 1 eq) in EtOAc (500 mL) was added PTSA (11.93 g, 69.26 mmol, 3 eq). The resulted reaction mixture was stirred at 70° C. for 3 hours. LCMS showed 6m was consumed, and desired mass was detected. Saturated NaHCO₃ (500 mL) was added to the reaction mixture and the organic layer was separated and concentrated to give crude 6f (TsOH salt) as a white solid. The crude product was used in the next step without purification.

### Example 7 (General Procedure G)

### (R)-5-(4-(6-((4-cyano-2-fluorobenzyl)oxy)pyridin-2-yl)piperidin-1-yl)-1,2,4,5-tetrahydrobenzo[4,5]imidazo[1,2-d][1,4]oxazepine-9-carboxylic acid and (S)-5-(4-(6-((4-cyano-2-fluorobenzyl)oxy)pyridin-2-yl)piperidin-1-yl)-1,2,4,5-tetrahydrobenzo[4,5]imidazo[1,2-d][1,4]oxazepine-9-carboxylic acid

The title compound was prepared according to Scheme 5. This General Procedure G exemplifies Scheme 5 and provides particular synthetic details as applied to the title compound.

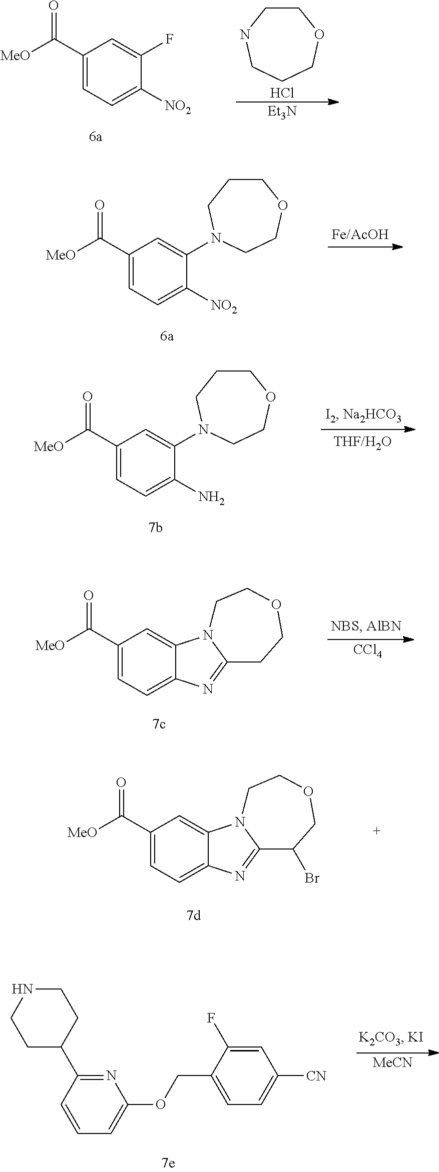

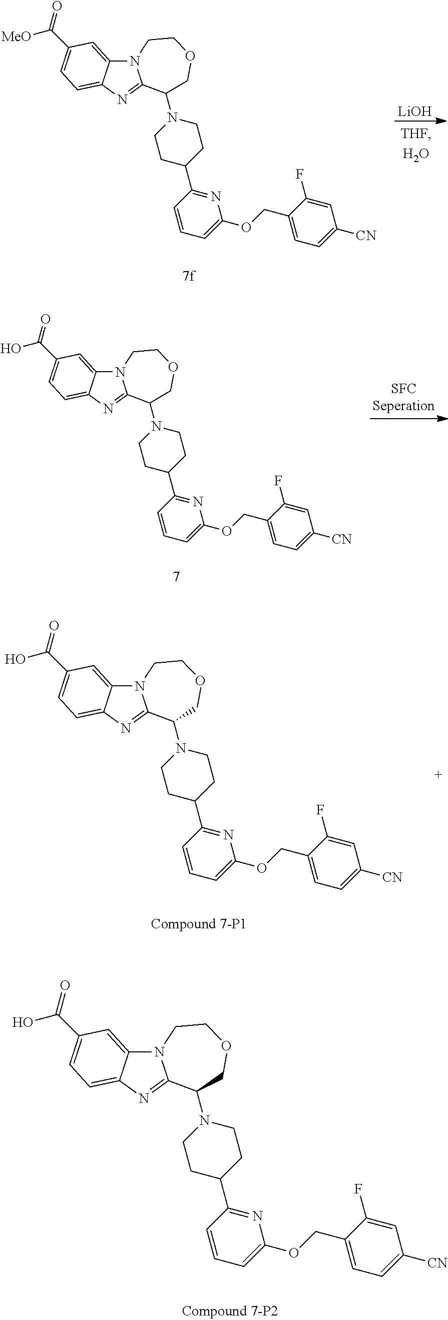 [SMILES: COC(=O)c1ccc2nc3n(c2c1)CCOCC3N1CCC(c2cccc(OCc3ccc(C#N)cc3F)n2)CC1 ; "LiOH" ; N#Cc1ccc(COc2cccc(C3CCN(C4COCCn5c4nc4ccc(C(=O)O)cc45)CC3)n2)c(F)c1 ; N#Cc1ccc(COc2cccc(C3CCN([C@H]4COCCn5c4nc4ccc(C(=O)O)cc45)CC3)n2)c(F)c1 ; N#Cc1ccc(COc2cccc(C3CCN([C@@H]4COCCn5c4nc4ccc(C(=O)O)cc45)CC3)n2)c(F)c1]

### Methyl 4-nitro-3-(1,4-oxazepan-4-yl)benzoate (7a)

To a solution of methyl 3-fluoro-4-nitrobenzoate (6a, 2 g, 10.04 mmol, 1 eq) and 1,4-oxazepane (3.50 g, 20.09 mmol, 2 eq, HCl) in THF (15 mL) was added Et3N (8.13 g, 80.35 mmol, 11.18 mL, 8 eq). The mixture was stirred at 25° C. for 16 hours. TLC (Petroleum ether:Ethyl acetate=4:1) indicated 6a was consumed, and one major new spot was formed. The reaction mixture was poured into water (50 mL), and extracted with ethyl acetate (50 mL*2). The combined organic phase was washed with brine (20 mL), dried over with anhydrous Na2SO4, filtered and concentrated in vacuo to give 7a as a yellow solid. 1H NMR (400 MHz, CDCl3-d) δ 7.78 (d, J=1.4 Hz, 1H), 7.72 (d, J=8.6 Hz, 1H), 7.48 (dd, J=1.2, 8.5 Hz, 1H), 3.94 (s, 3H), 3.88-3.82 (m, 4H), 3.82-3.78 (m, 1H), 3.50-3.40 (m, 4H), 3.02-2.96 (m, 1H).

### Methyl 4-amino-3-(1,4-oxazepan-4-yl)benzoate (7b)

To a solution of methyl 4-nitro-3-(1,4-oxazepan-4-yl)benzoate (7a, 2.8 g, 9.99 mmol, 1 eq) in AcOH (10 mL) was added Fe (5.58 g, 99.90 mmol, 10 eq). The mixture was stirred at 35° C. for 2 hours. TLC (Petroleum ether:Ethyl acetate=4:1) indicated 7a was consumed, and a new spot was formed. The reaction mixture was diluted with Ethyl acetate (20 mL) and filtered. The filtrate was adjusted pH=7 with saturated NaHCO₃ solution. The mixture was extracted with Ethyl acetate (30 mL *2). The combined organic layers were dried over Mg2SO4, filtered and concentrated under reduced pressure to give 7b as a white solid. 1H NMR (400 MHz, CDCl3-d) δ 7.75 (d, J=1.8 Hz, 1H), 7.65 (dd, J=1.8, 8.2 Hz, 1H), 6.70 (d, J=8.2 Hz, 1H), 4.46 (br s, 2H), 3.88-3.82 (m, 5H), 3.16-3.09 (m, 4H), 2.02 (quin, J=5.8 Hz, 2H).

### Methyl 1,2,4,5-tetrahydrobenzo[4,5]imidazo[1,2-d][1,4]oxazepine-9-carboxylate (7c)

To a solution of methyl 4-amino-3-(1,4-oxazepan-4-yl)benzoate (7b, 2.3 g, 9.19 mmol, 1 eq) in THF (15 mL) H2O (5 mL) was added 12 (17.49 g, 68.92 mmol, 13.88 mL, 7.5 eq) and NaHCO3 (7.72 g, 91.89 mmol, 3.57 mL, 10 eq). The mixture was stirred at 25° C. for 5 hours. LCMS showed of 7b was consumed, and desired mass was detected. The reaction mixture was poured into water, and quenched by addition of Na2S203 (100 mL, aq) at 25° C. The aqueous phase was extracted with ethyl acetate (150 mL*2). The combined organic phase was washed with brine (50 mL), dried over with anhydrous Na2SO4, filtered and concentrated in vacuo. The crude product was purified by reversed-phase HPLC ([water (10 mM NH4HCO3)-ACN]).column: Agela DuraShell C18 250*70 mm*10 um; mobile phase: [water (10 mM NH4HCO3)-ACN]; B %: 17%-37%, 22 min to give 7c as a white solid. 1H NMR (400 MHz, CDCl3-d) δ 8.03 (s, 1H), 7.97 (d, J=8.4 Hz, 1H), 7.72 (d, J=8.4 Hz, 1H), 4.40-4.32 (m, 2H), 4.02-3.93 (m, 7H), 3.42-3.36 (m, 2H).

### Methyl 5-bromo-1,2,4,5-tetrahydrobenzo[4,5]imidazo[1,2-d][1,4]oxazepine-9-carboxylate (7d)

To a solution of methyl 1,2,4,5-tetrahydrobenzo[4,5]imidazo[1,2-d][1,4]oxazepine-9-carboxylate (7c, 300 mg, 1.22 mmol, 1 eq) in CCl4 (8 mL) was added NBS (303.55 mg, 1.71 mmol, 1.4 eq) and AIBN (80.02 mg, 487.29 umol, 0.4 eq). The mixture was stirred at 80° C. for 5 hours. TLC (Petroleum ether:Ethyl acetate=1:2) indicated 7c remained, and one major new spot was formed. The reaction mixture was diluted with water (60 mL) and extracted with Ethyl acetate (40 mL*2). The combined organic layers were washed with brine (20 mL*1), dried over Na2SO4, filtered and concentrated under reduced pressure. The residue was purified by column chromatography (SiO2, petroleum ether:ethyl acetate=10:1 to 0:1) to give 7d as a white solid. 1H NMR (400 MHz, CDCl3-d) δ 8.10 (s, 1H), 8.01 (dd, J=1.4, 8.6 Hz, 1H), 7.79 (d, J=8.6 Hz, 1H), 5.59 (d, J=2.6 Hz, 1H), 4.63-4.55 (m, 1H), 4.52-4.44 (m, 2H), 4.36 (dd, J=3.8, 13.7 Hz, 1H), 4.00 (d, J=13.6 Hz, 1H), 3.97 (s, 3H), 3.78-3.66 (m, 1H).

### Methyl 5-(4-(6-((4-cyano-2-fluorobenzyl)oxy)pyridin-2-yl)piperidin-1-yl)-1,2,4,5-tetrahydrobenzo[4,5]imidazo[1,2-d][1,4]oxazepine-9-carboxylate (7f)

To a solution of methyl 5-bromo-1,2,4,5-tetrahydrobenzo[4,5]imidazo[1,2-d][1,4]oxazepine-9-carboxylate (7d, 400 mg, 1.23 mmol, 1 eq) 3-fluoro-4-(((6-(piperidin-4-yl)pyridin-2-yl)oxy)methyl)benzonitrile (7e, 574.53 mg, 1.85 mmol, 1.5 eq) in ACN (10 mL) was added KI (306.32 mg, 1.85 mmol, 1.5 eq) and K2CO3 (255.03 mg, 1.85 mmol, 1.5 eq). The mixture was stirred at 50° C. for 5 hours. TLC (Petroleum ether:Ethyl acetate=1:2) indicated of 7d was remained, and new spot was formed. The reaction mixture was diluted with water (30 mL) and extracted with Ethyl acetate (30 mL*2). The combined organic layers were washed with brine (10 mL), dried over Na2SO4, filtered and concentrated under reduced pressure. The residue was purified by column chromatography (SiO2, Petroleum ether:Ethyl acetate=10:1 to 0:1) to give 7f as a white solid. 1H NMR (400 MHz, CDCl3-d) δ 8.09 (s, 1H), 7.99 (d, J=8.4 Hz, 1H), 7.76 (d, J=8.6 Hz, 1H), 7.63 (t, J=7.0 Hz, 1H), 7.52 (t, J=7.8 Hz, 1H), 7.44 (d, J=7.6 Hz, 1H), 7.37 (d, J=9.0 Hz, 1H), 6.76 (d, J=7.2 Hz, 1H), 6.64 (d, J=8.2 Hz, 1H), 5.50 (s, 2H), 5.02-4.91 (m, 1H), 4.50 (dd, J=4.0, 13.4 Hz, 1H), 4.32 (br d, J=13.8 Hz, 2H), 3.97 (s, 3H), 3.73-3.65 (m, 1H), 3.65-3.50 (m, 2H), 2.79 (s, 1H), 2.75-2.57 (m, 2H), 2.33-2.18 (m, 2H), 2.00-1.82 (m, 2H), 1.82-1.61 (m, 2H).

### 5-(4-(6-((4-cyano-2-fluorobenzyl)oxy)pyridin-2-yl)piperidin-1-yl)-1,2,4,5-tetrahydrobenzo[4,5]imidazo[1,2-d][1,4]oxazepine-9-carboxylic acid (7)

To a solution of methyl 5-(4-(6-(benzyloxy)pyridin-2-yl)piperidin-1-yl)-1,2,4,5-tetrahydrobenzo[4,5]imidazo[1,2-d][1,4]oxazepine-9-carboxylate (7f, 380 mg, 683.95 umol, 1 eq) in THF (21 mL), H2O (9 mL) was added LiOH·H2O (28.70 mg, 683.95 umol, 1 eq). The mixture was stirred at 20° C. for 16 hours. LCMS showed 7f remained, and desired mass was detected. Then LiOH·H2O (14.35 mg, 341.97 umol, 0.5 eq) was added in the mixture. The mixture was stirred at 20° C. for another 16 hours. LCMS showed most of 7f was consumed, filtered and contracted under reduced pressure. The residue was purified by prep-HPLC (column: Waters Xbridge Prep OBD C18 150*40 mm*10 um; mobile phase: [water (10 mM NH4HCO3)-ACN]; B %: 10%-45%, 8 min) to give Compound 7 as a white solid. 1H NMR (400 MHz, CDCl3-d) δ 8.16 (s, 1H), 8.07 (dd, J=1.6, 8.6 Hz, 1H), 7.83 (d, J=8.4 Hz, 1H), 7.63 (t, J=7.4 Hz, 1H), 7.52 (t, J=7.4 Hz, 1H), 7.44 (d, J=7.6 Hz, 1H), 7.37 (d, J=9.4 Hz, 1H), 6.76 (d, J=7.4 Hz, 1H), 6.64 (d, J=8.2 Hz, 1H), 5.54-5.46 (m, 2H), 5.02-4.94 (m, 1H), 4.52 (dd, J=4.2, 13.8 Hz, 1H), 4.34 (d, J=12.6 Hz, 2H), 3.76-3.68 (m, 2H), 3.67-3.50 (m, 2H), 2.72 (br d, J=12.2 Hz, 1H), 2.67-2.57 (m, 1H), 2.36-2.30 (m, 1H), 2.30-2.18 (m, 2H), 1.99-1.87 (m, 2H), 1.83-1.64 (m, 2H).

### (R)-5-(4-(6-((4-cyano-2-fluorobenzyl)oxy)pyridin-2-yl)piperidin-1-yl)-1,2,4,5-tetrahydrobenzo[4,5]imidazo[1,2-d][1,4]oxazepine-9-carboxylic acid and (S)-5-(4-(6-((4-cyano-2-fluorobenzyl)oxy)pyridin-2-yl)piperidin-1-yl)-1,2,4,5-tetrahydrobenzo[4,5]imidazo[1,2-d][1,4]oxazepine-9-carboxylic acid (Compounds 7-P1 & 7-P2)

The compound 7 was purified by Chiral SFC (Thar SFC80 preparative SFC; Column: Chiralpak OD 250*30 mm i.d. 10u; Mobile phase: A for C02 and B for MeOH (0.1% NH3H2O); Gradient: B %=50%; Flow rate: 70 g/min; Wavelength: 220 nm; Column temperature: 40° C.; System back pressure: 100 bar) to give Compound 7-P1 as a white solid. MS mass calculated for [M+1]+(C30H28FN5O4) requires m/z 542.2, LCMS found m/z 542.3; 1H NMR (400 MHz, CDCl3-d) δ 8.19 (s, 1H), 8.09 (br d, J=8.4 Hz, 1H), 7.85 (d, J=8.4 Hz, 1H), 7.63 (t, J=7.6 Hz, 1H), 7.52 (t, J=7.8 Hz, 1H), 7.43 (d, J=7.8 Hz, 1H), 7.37 (d, J=9.4 Hz, 1H), 6.76 (d, J=7.4 Hz, 1H), 6.64 (d, J=8.2 Hz, 1H), 5.57-5.44 (m, 2H), 5.06-4.93 (m, 1H), 4.53 (br dd, J=3.8, 13.6 Hz, 1H), 4.44-4.26 (m, 2H), 3.82-3.69 (m, 2H), 3.63 (br t, J=11.8 Hz, 1H), 3.54 (br d, J=10.2 Hz, 1H), 2.73 (br d, J=11.4 Hz, 1H), 2.68-2.56 (m, 1H), 2.39-2.19 (m, 2H), 2.01-1.85 (m, 2H), 1.84-1.63 (m, 2H).

Compound 7-P2 was obtained as a white solid. MS mass calculated for [M+1]+(C30H28FN5O4) requires m/z 542.2, LCMS found m/z 542.3; 1H NMR (400 MHz, CDCl3-d) δ 8.18 (s, 1H), 8.09 (br d, J=8.4 Hz, 1H), 7.85 (br d, J=8.4 Hz, 1H), 7.63 (t, J=7.4 Hz, 1H), 7.52 (t, J=7.8 Hz, 1H), 7.44 (d, J=7.8 Hz, 1H), 7.37 (d, J=9.4 Hz, 1H), 6.76 (d, J=7.4 Hz, 1H), 6.64 (d, J=8.2 Hz, 1H), 5.56-5.45 (m, 2H), 5.04-4.93 (m, 1H), 4.53 (br dd, J=3.8, 13.6 Hz, 1H), 4.35 (br d, J=13.4 Hz, 2H), 3.79-3.69 (m, 2H), 3.63 (br t, J=11.4 Hz, 1H), 3.54 (br d, J=10.4 Hz, 1H), 2.72 (br d, J=11.2 Hz, 1H), 2.67-2.58 (m, 1H), 2.38-2.20 (m, 2H), 2.00-1.86 (m, 2H), 1.86-1.61 (m, 2H).

When a mixture of stereoisomers is separated by HPLC, it is to be appreciated that the resultant individual stereoisomers or mixtures will be arbitrarily assigned. In the examples described herein, when the mixture of stereoisomers is separated by HPLC, it is to be appreciated that an eluting enantiomer or an enantiomer of a resulting compound prepared from the eluting enantiomer is labeled “P1” and another eluting enantiomer or an enantiomer of a resulting compound prepared from the another eluting enantiomer is labeled “P2”. In this example, the eluting enantiomers are of Compound 7. The absolute configuration of the enantiomers, e.g., Compounds 7-P1 & 7-P2 each associated with the corresponding 1H NMR data, may be obtained by known methods.

### Example 8 (General Procedure H)

### (1S,5R)-5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylic acid and (1S,5S)-5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylic acid

The title compound was prepared according to Scheme 5. This General Procedure H exemplifies Scheme 5 and provides particular synthetic details as applied to the title compound.

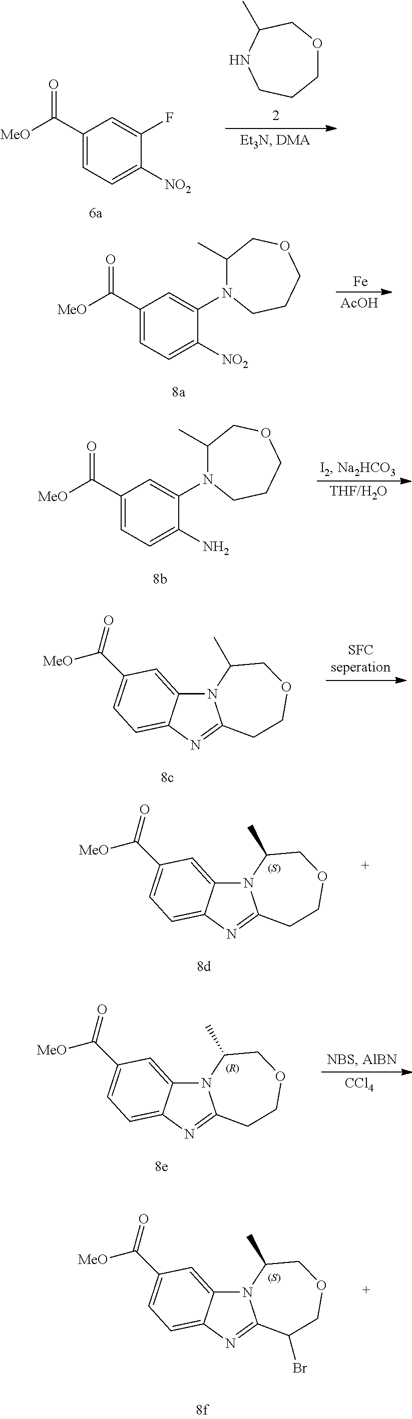

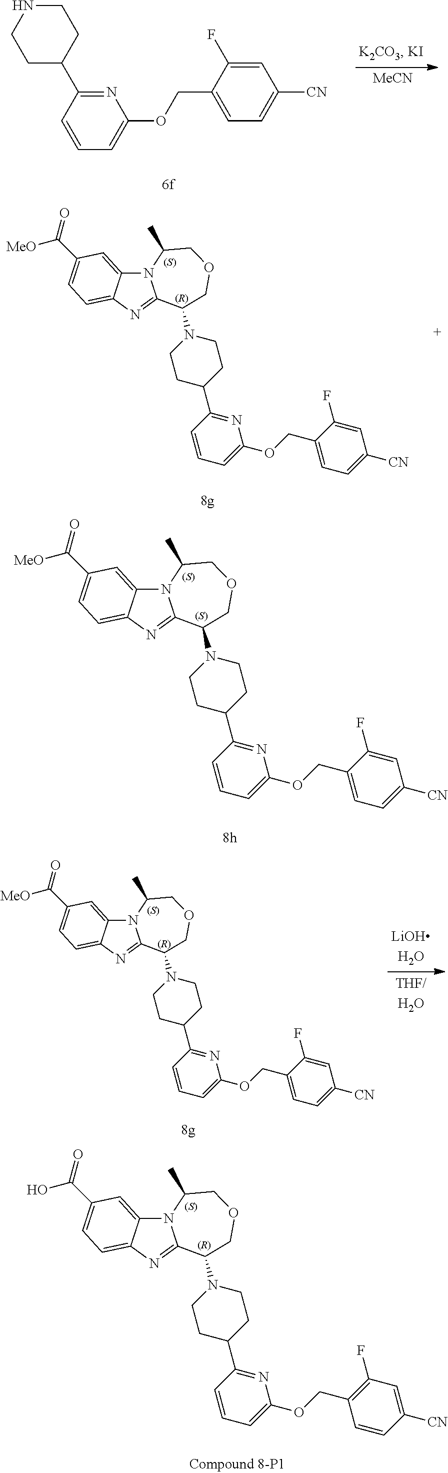

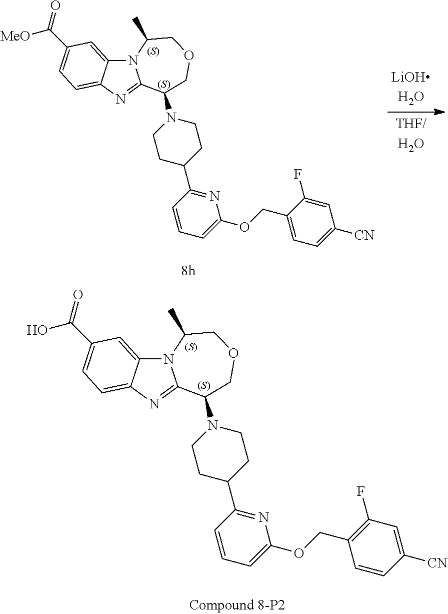

### Methyl 3-(3-methyl-1,4-oxazepan-4-yl)-4-nitrobenzoate (8a)

To a solution of methyl 3-fluoro-4-nitrobenzoate (6a, 700 mg, 6.08 mmol, 1 eq) and 3-methyl-1,4-oxazepane (1.82 g, 9.12 mmol, 1.5 eq) in DMA (15 mL) was added Et3N (1.23 g, 12.16 mmol, 1.69 mL, 2 eq). The mixture was stirred at 80° C. for 16 hours. TLC (Dichloromethane:Methanol=1:1) indicated 6a was consumed and one new spot was formed. The reaction mixture was extracted with Ethyl acetate (20 mL*2) and water (45 mL). The combined organic layers were washed with brine (10 mL *2), dried over Na2SO4, filtered and concentrated under reduced pressure. The residue was purified by column chromatography (SiO2, Petroleum ether:Ethyl acetate=10:1 to 1:1) to give 8a as yellow oil. 1H NMR (400 MHz, CDCl3-d) δ 7.97 (s, 1H), 7.65 (s, 2H), 3.97-3.70 (m, 7H), 3.56 (dd, J=7.0, 13.2 Hz, 1H), 3.41 (ddd, J=2.8, 8.8, 14.4 Hz, 1H), 3.20 (ddd, J=3.2, 7.0, 14.4 Hz, 1H), 2.00-1.81 (m, 2H), 1.07 (d, J=6.6 Hz, 3H).

### Methyl 4-amino-3-(3-methyl-1,4-oxazepan-4-yl)benzoate (8b)

To a solution of Methyl 3-(3-methyl-1,4-oxazepan-4-yl)-4-nitrobenzoate (8a, 1.4 g, 4.76 mmol, 1 eq) in AcOH (15 mL) was added Fe (2.66 g, 47.57 mmol, 10 eq). The mixture was stirred at 35° C. for 2 hours. LCMS showed of 8a was consumed and desired mass was detected. The reaction mixture was diluted with Ethyl acetate (40 mL) and filtered. The filtrate was adjusted to pH=8 with saturated NaHCO3 (aq) and extracted with Ethyl acetate (80 mL). The organic layer was dried over Na2SO4, filtered and concentrated under reduced pressure. The residue was purified by column chromatography (SiO2, Petroleum ether:Ethyl acetate=10:1 to 0:1) to give 8b as a white solid. 1H NMR (400 MHz, CDCl3-d) δ 7.81 (d, J=1.8 Hz, 1H), 7.66 (dd, J=2.0, 8.4 Hz, 1H), 6.70 (d, J=8.4 Hz, 1H), 4.55 (br s, 2H), 3.96-3.88 (m, 2H), 3.86 (s, 3H), 3.78 (td, J=6.2, 11.8 Hz, 1H), 3.57-3.42 (m, 2H), 3.23-3.09 (m, 2H), 2.01-1.86 (m, 2H), 0.88 (d, J=6.4 Hz, 3H).

### Methyl 1-methyl-1,2,4,5-tetrahydrobenzo[4,5]imidazo[1,2-d][1,4]oxazepine-9-carboxylate (8c)

To a solution of methyl 4-amino-3-(3-methyl-1,4-oxazepan-4-yl)benzoate (8b, 450 mg, 1.70 mmol, 1 eq) in THF (9 mL) and H2O (3 mL) was added 12 (3.24 g, 12.75 mmol, 2.57 mL, 7.5 eq) and NaHCO3 (1.43 g, 17.00 mmol, 661.19 uL, 10 eq). The mixture was stirred at 20° C. for 5 hours. TLC (Ethyl acetate:Methanol=10:1) indicated 8b was consumed, and one major new spot was formed. The reaction mixture was quenched by addition of Na2S2SO3 (100 mL) at 20° C., and extracted with Ethyl acetate (80 mL*2). The combined organic layers were washed with brine (30 mL*2), dried over Na2SO4, filtered and concentrated under reduced pressure. The residue was purified by column chromatography (SiO2, Ethyl acetate:Methanol=30:1 to 5:1) to give 8c as a brown solid. 1H NMR (400 MHz, CDCl3-d) δ 8.06 (s, 1H), 7.97 (dd, J=1.6, 8.4 Hz, 1H), 7.71 (d, J=8.6 Hz, 1H), 4.68-4.60 (m, 1H), 4.36-4.22 (m, 2H), 3.96 (s, 3H), 3.89-3.77 (m, 1H), 3.66 (ddd, J=2.4, 10.6, 12.4 Hz, 1H), 3.49-3.36 (m, 2H), 1.60 (d, J=7.2 Hz, 3H).

### (S)-methyl 1-methyl-1,2,4,5-tetrahydrobenzo[4,5]imidazo[1,2-d][1,4]oxazepine-9-carboxylate (8d) & (R)-methyl 1-methyl-1,2,4,5-tetrahydrobenzo[4,5]imidazo[1,2-d][1,4]oxazepine-9-carboxylate (8e)

Methyl 1-methyl-1,2,4,5-tetrahydrobenzo[4,5]imidazo[1,2-d][1,4]oxazepine-9-carboxylate (8c) was separated by Chiral SFC (Chiralpak AD, 250*30 mm i.d. 10 um; Mobile phase: A for C02 and B for EtOH; Gradient: B %=25% isocratic elution mode; Flow rate: 60 g/min; Wavelength: 220 nm; Column temperature: 35° C.; System back pressure: 100 bar) to give 8d as a white solid. 1H NMR (400 MHz, CDCl3-d) δ 8.06 (s, 1H), 7.98 (dd, J=1.6, 8.4 Hz, 1H), 7.71 (d, J=8.6 Hz, 1H), 4.67-4.60 (m, 1H), 4.36-4.21 (m, 2H), 3.96 (s, 3H), 3.89-3.76 (m, 1H), 3.66 (ddd, J=2.4, 10.5, 12.5 Hz, 1H), 3.48-3.36 (m, 2H), 1.60 (d, J=7.2 Hz, 3H).

8e was obtained as a white solid. 1H NMR (400 MHz, CDCl3-d) δ 8.06 (s, 1H), 7.98 (dd, J=1.6, 8.4 Hz, 1H), 7.71 (d, J=8.6 Hz, 1H), 4.68-4.61 (m, 1H), 4.36-4.21 (m, 2H), 3.96 (s, 3H), 3.89-3.76 (m, 1H), 3.66 (ddd, J=2.4, 10.6, 12.4 Hz, 1H), 3.49-3.35 (m, 2H), 1.60 (d, J=7.2 Hz, 3H).

### (1S)-methyl 5-bromo-1-methyl-1,2,4,5-tetrahydrobenzo[4,5]imidazo[1,2-d][1,4]oxazepine-9-carboxylate (8f)

To a solution of (S)-methyl 1-methyl-1,2,4,5-tetrahydrobenzo[4,5]imidazo[1,2-d][1,4]oxazepine-9-carboxylate (8d, 150.00 mg, 576.29 umol, 1 eq) in CCl4 (5 mL) was added AIBN (37.85 mg, 230.51 umol, 0.4 eq) and NBS (143.60 mg, 806.80 umol, 1.4 eq). The mixture was stirred at 80° C. for 5 hours. TLC (Petroleum ether:Ethyl acetate=1:1) indicated 8d was consumed, and one new spot was formed. The reaction mixture was filtered and concentrated under reduced pressure. The residue was purified by prep-TLC (SiO2, Petroleum ether:Ethyl acetate=1:1) to give 8f as a yellow solid. 1H NMR (400 MHz, CDCl3-d) δ 8.12 (s, 1H), 8.02 (dd, J=1.6, 8.6 Hz, 1H), 7.77 (d, J=8.4 Hz, 1H), 5.69 (dd, J=1.4, 2.4 Hz, 1H), 4.76-4.69 (m, 1H), 4.49 (dd, J=2.6, 13.8 Hz, 1H), 4.41 (dd, J=2.4, 13.2 Hz, 1H), 4.07 (dd, J=1.4, 13.8 Hz, 1H), 3.98 (s, 3H), 3.95-3.90 (m, 1H), 1.84 (d, J=7.2 Hz, 3H).

### (1S,5R)-methyl 5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylate (8g) & (1S,5S)-methyl 5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylate (8h)

To a solution of (1S)-methyl 5-bromo-1-methyl-1,2,4,5-tetrahydrobenzo[4,5]imidazo[1,2-d][1,4]oxazepine-9-carboxylate (8f, 40 mg, 117.93 umol, 1 eq) and 3-fluoro-4-(((6-(piperidin-4-yl)pyridin-2-yl)oxy)methyl)benzonitrile (6f, 40.39 mg, 129.72 umol, 1.1 eq) in CH3CN (3 mL) was added K2CO3 (24.45 mg, 176.90 umol, 1.5 eq) and KI (29.36 mg, 176.90 umol, 1.5 eq). The mixture was stirred at 50° C.-65° C. for 16 hours. LCMS showed 8f was consumed and desired mass was detected. The reaction mixture was filtered, and the fitrate was concentrated under reduced pressure. The residue was purified by prep-TLC (SiO2, Petroleum ether:Ethyl acetate=1:2) to give 8g as a white solid. 1H NMR (400 MHz, CDCl3-d) δ 8.11 (s, 1H), 7.97 (dd, J=1.4, 8.4 Hz, 1H), 7.79 (d, J=8.4 Hz, 1H), 7.66 (t, J=7.4 Hz, 1H), 7.55 (t, J=7.8 Hz, 1H), 7.47 (d, J=7.8 Hz, 1H), 7.39 (d, J=9.4 Hz, 1H), 6.82 (d, J=7.2 Hz, 1H), 6.66 (d, J=8.2 Hz, 1H), 5.54 (s, 2H), 4.83 (br d, J=7.2 Hz, 1H), 4.37 (dd, J=1.8, 12.0 Hz, 1H), 4.20 (dd, J=2.0, 8.4 Hz, 1H), 4.15-4.09 (m, 1H), 4.06-4.00 (m, 1H), 3.96 (s, 3H), 3.89 (dd, J=8.4, 12.1 Hz, 1H), 3.44 (br s, 1H), 3.06 (br s, 1H), 2.98 (br s, 1H), 2.77 (br d, J=3.2 Hz, 2H), 2.03-1.88 (m, 4H), 1.63 (d, J=7.2 Hz, 3H)

Compound 8h was obtained as a white solid. 1H NMR (400 MHz, CDCl3-d) δ 8.10 (s, 1H), 7.99 (dd, J=1.4, 8.4 Hz, 1H), 7.77 (d, J=8.6 Hz, 1H), 7.60 (t, J=7.2 Hz, 1H), 7.51 (dd, J=7.4, 8.2 Hz, 1H), 7.42 (d, J=8.2 Hz, 1H), 7.36 (d, J=9.2 Hz, 1H), 6.74 (d, J=7.4 Hz, 1H), 6.63 (d, J=7.8 Hz, 1H), 5.49 (s, 2H), 4.61 (br dd, J=3.2, 13.8 Hz, 2H), 4.28 (dd, J=2.4, 13.1 Hz, 1H), 3.97 (s, 3H), 3.82 (br d, J=12.2 Hz, 1H), 3.74-3.50 (m, 2H), 2.61 (br s, 2H), 2.18 (s, 2H), 1.97 (d, J=7.2 Hz, 4H), 1.38-1.16 (m, 4H).

### (1S,5R)-5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylic acid (Compound 8-P1)

To a solution of (1S,5R)-methyl 5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylate (8g, 40 mg, 70.22 umol, 1 eq) in THF (1.7 mL) and H2O (0.7 mL) was added LiOH·H2O (5.89 mg, 140.44 umol, 2 eq). The mixture was stirred at 20° C. for 16 hours. LCMS showed 8g was remained and desired compound was detected. The mixture was adjusted to pH=6 with Citric acid (aq, 1 M). The mixture was concentrated under reduced pressure to remove THF, then diluted with water (5 mL) and extracted with Ethyl acetate (10 Ml). The combined organic layers were dried over Na2SO4, filtered and concentrated under reduced pressure. The residue was purified by prep-TLC (SiO₂, Petroleum ether:Ethyl acetate=0:1) to give 8-P1 as a white solid. MS mass calculated for [M+1]+(C31H30FN5O4) requires m/z 556.2, LCMS found m/z 556.2; 1H NMR (400 MHz, CDCl3-d) δ 8.19 (s, 1H), 8.05 (br d, J=8.6 Hz, 1H), 7.83 (br d, J=8.4 Hz, 1H), 7.66 (br t, J=7.6 Hz, 1H), 7.54 (br t, J=7.8 Hz, 1H), 7.49-7.43 (m, 1H), 7.43-7.33 (m, 1H), 6.81 (br d, J=7.4 Hz, 1H), 6.66 (d, J=8.4 Hz, 1H), 5.53 (s, 2H), 4.85 (br s, 1H), 4.39 (br d, J=11.6 Hz, 1H), 4.24 (br d, J=7.8 Hz, 1H), 4.12 (br d, J=12.6 Hz, 1H), 4.08-3.98 (m, 1H), 3.98-3.84 (m, 1H), 3.46 (br d, J=8.8 Hz, 1H), 3.13-2.94 (m, 2H), 2.83-2.64 (m, 2H), 2.07-1.85 (m, 4H), 1.64 (br d, J=7.0 Hz, 3H).

### (1S,5S)-5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylic acid (Compound 8-P2)

To a solution of (1S,5S)-methyl 5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylate (8h, 6 mg, 10.53 umol, 1 eq) in THF (0.7 mL) and H2O (0.3 mL) was added LiOH·H2O (1.19 mg, 28.44 umol, 2.7 eq). The mixture was stirred at 20° C. for 16 hours. LCMS showed 8h was consumed and desired mass was detected. The mixture was adjusted to pH=6 with Citric acid (aq, 1 M). The mixture was concentrated under reduced pressure to remove THF, then diluted with water (5 mL) and extracted with Ethyl acetate (5 mL). The combined organic layers were dried over Na2SO4, filtered and concentrated under reduced pressure. The residue was purified by prep-TLC (SiO₂, Petroleum ether:Ethyl acetate=0:1) to give 8-P2 as a white solid. MS mass calculated for [M+1]+(C31H30FN5O4) requires m/z 556.2, LCMS found m/z 556.2; 1H NMR (400 MHz, CDCl3-d) δ 8.19 (s, 1H), 8.12-8.05 (m, 1H), 7.83 (d, J=8.4 Hz, 1H), 7.61 (t, J=7.4 Hz, 1H), 7.51 (t, J=7.8 Hz, 1H), 7.42 (d, J=7.6 Hz, 1H), 7.36 (d, J=9.4 Hz, 1H), 6.75 (d, J=7.4 Hz, 1H), 6.63 (d, J=8.2 Hz, 1H), 5.49 (s, 2H), 4.63 (br d, J=10.8 Hz, 1H), 4.30 (br d, J=11.8 Hz, 1H), 3.89-3.79 (m, 1H), 3.76 (br s, 1H), 3.72-3.54 (m, 2H), 2.69-2.56 (m, 2H), 2.33-2.19 (m, 1H), 2.33-2.19 (m, 1H), 1.99 (br d, J=7.0 Hz, 4H), 1.77 (br d, J=12.6 Hz, 1H), 1.72-1.53 (m, 3H).

When a mixture of stereoisomers is separated by HPLC, it is to be appreciated that the resultant individual stereoisomers or mixtures will be arbitrarily assigned. In the examples described herein, when the mixture of stereoisomers is separated by HPLC, it is to be appreciated that an eluting enantiomer or an enantiomer of a resulting compound prepared from the eluting enantiomer is labeled “P1” and another eluting enantiomer or an enantiomer of a resulting compound prepared from the another eluting enantiomer is labeled “P2”. In this example, the eluting enantiomers are 8d and 8e, and the resulting compound is Compound 8. The absolute configuration of the enantiomers, e.g., 8d & 8e, as well as Compounds 8-P1 & 8-P2 each associated with the corresponding 1H NMR data, may be obtained by known methods.

### Example 9 (General Procedure I)

### 2-((6-(6-((4-cyano-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylic acid

### 2-(((1R,6S)-6-(6-((4-cyano-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylic acid

### and

### 2-(((1S,6R)-6-(6-((4-cyano-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylic acid

The title compound was prepared according to Scheme 6. This General Procedure I exemplifies Scheme 6 and provides particular synthetic details as applied to the title compound.

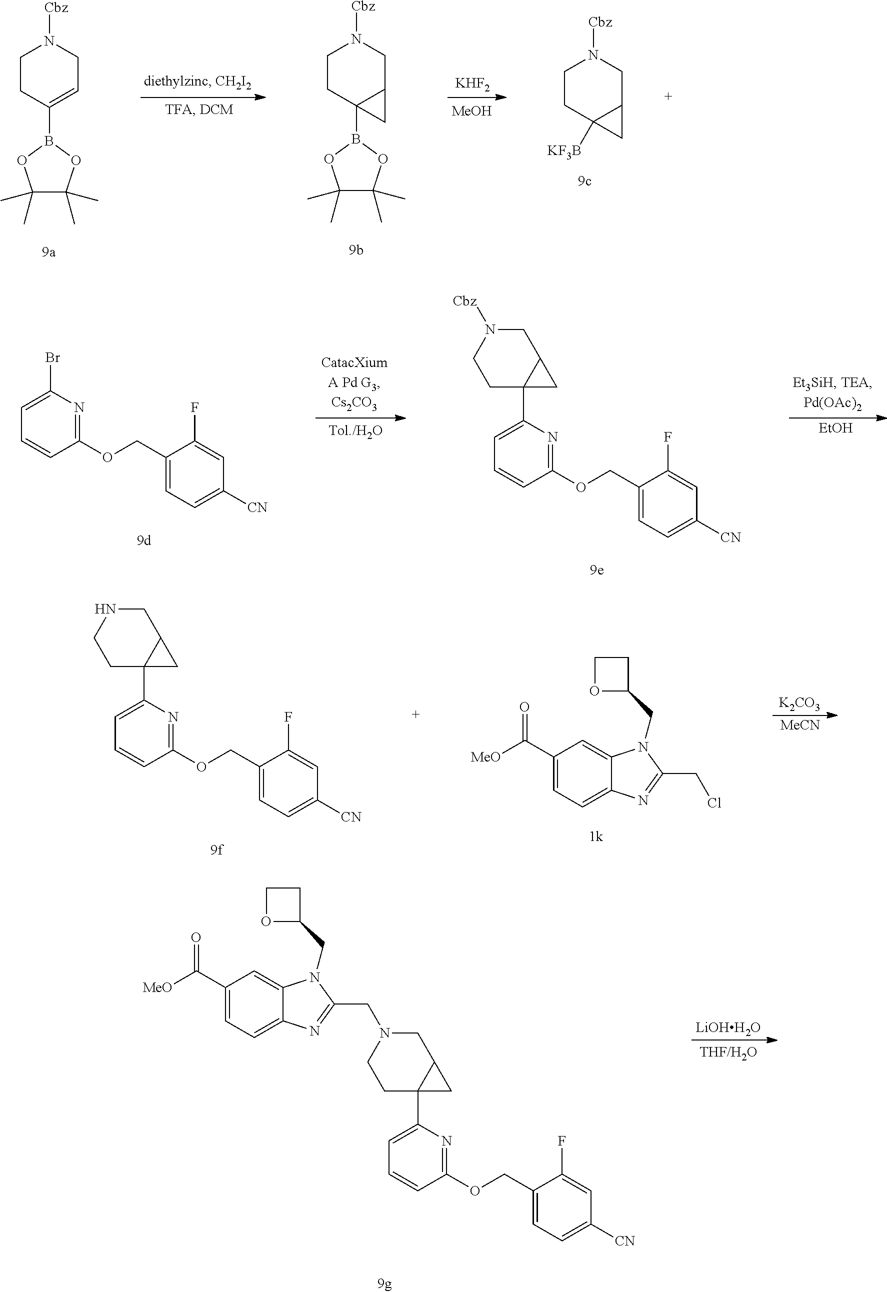

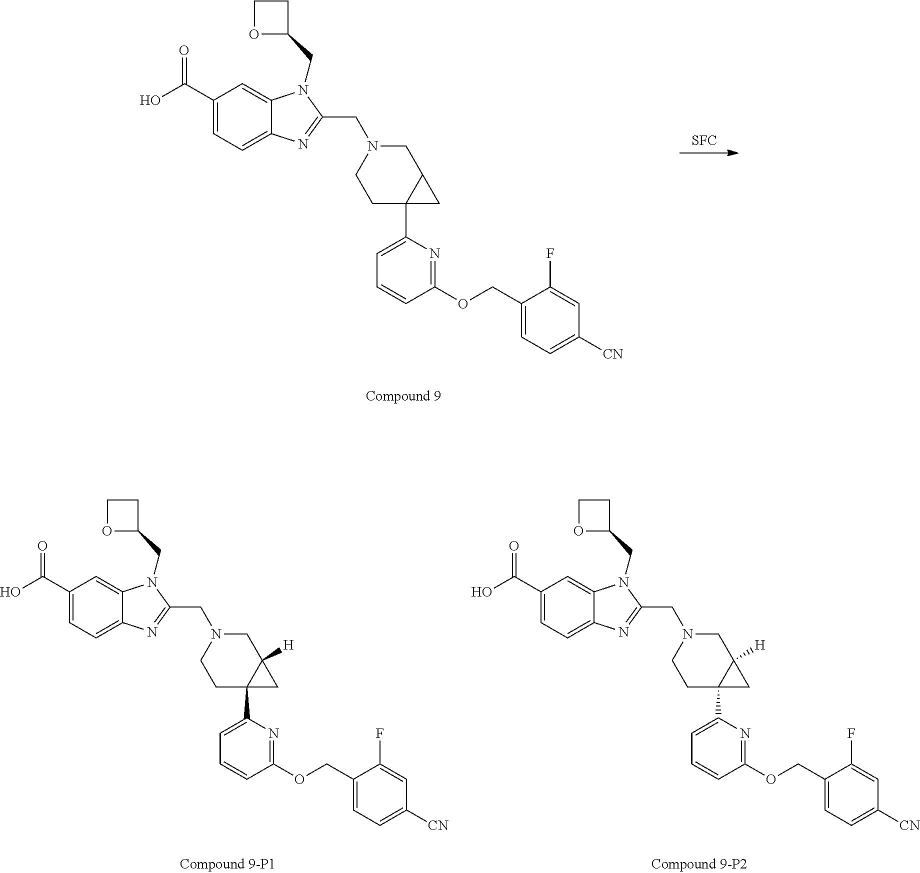 [SMILES: N#Cc1ccc(COc2cccc(C34CCN(Cc5nc6ccc(C(=O)O)cc6n5C[C@@H]5CCO5)CC3C4)n2)c(F)c1 ; "SFC" ; N#Cc1ccc(COc2cccc([C@@]34CCN(Cc5nc6ccc(C(=O)O)cc6n5C[C@@H]5CCO5)C[C@@H]3C4)n2)c(F)c1 ; N#Cc1ccc(COc2cccc([C@]34CCN(Cc5nc6ccc(C(=O)O)cc6n5C[C@@H]5CCO5)C[C@H]3C4)n2)c(F)c1]

### Benzyl 6-(4,4,5,5-tetramethyl-1,3,2-dioxaborolan-2-yl)-3-azabicyclo[4.1.0]heptane-3-carboxylate (9b)

To a solution of ZnEt2 (1 M, 23.31 mL, 8 eq) in DCM (15 mL) was added CH2I2 (12.49 g, 46.62 mmol, 3.76 mL, 16 eq) slowly at −40° C. and the mixture was stirred at −40° C. for 1 hour. TFA (2.66 g, 23.31 mmol, 1.73 mL, 8 eq) was added at −40° C. and the mixture was stirred at −15° C. for 1 hour. Then benzyl 4-(4,4,5,5-tetramethyl-1,3,2-dioxaborolan-2-yl)-5,6-dihydropyridine-1(2H)-carboxylate (9a, 1g, 2.91 mmol, 1 eq) in DCM (5 mL) was added to the reaction mixture slowly at −15° C. and the mixture was stirred at 25° C. for 16 hours. LCMS showed 9a was consumed, and desired MS was detected. The mixture was filtered and concentrated under reduced pressure. The residue was purified by column chromatography (SiO2, Petroleum ether:Ethyl acetate=10:1 to 1:1) to give 9b as a yellow solid. 1HNMR (400 MHz, CDCl3-d) δ 7.42-7.29 (m, 5H), 5.12 (s, 2H), 3.89 (br d, J=11.4 Hz, 1H), 3.65-3.40 (m, 2H), 2.95 (br s, 1H), 2.11 (br d, J=14.0 Hz, 1H), 1.26-1.17 (m, 14H), 0.90 (br s, 1H), 0.43 (br s, 1H).

### [(Z)-(3-benzyloxycarbonyl-3-azabicyclo[4.1.0]heptan-6-yl)boranylidene-fluoranyl]-difluoro-potassium (9c)

To a solution of benzyl 6-(4,4,5,5-tetramethyl-1,3,2-dioxaborolan-2-yl)-3-azabicyclo[4.1.0]heptane-3-carboxylate (9b, 250 mg, 699.79 umol, 1 eq) in MeOH (5 mL) was added KHF2 (382.57 mg, 4.90 mmol, 161.42 uL, 7 eq) at 25° C. The mixture was stirred at 90° C. for 16 hours. TLC (Petroleum ether:Ethyl acetate=3:1) showed 9b was consumed. The reaction mixture was concentrated under reduced pressure to remove MeOH. The solid was triturated with a solution of Petroleum ether:MTBE=5:1 (5 mL). The mixture was filtered; the filter cake was dried in vacuo to give crude product as a white solid. The crude product was dissolved in hot MeCN (5 mL) and filtered. The filtrate was concentrated under reduced pressure to give 9c as a white solid. 1H NMR (400 MHz, DMSO-d6) δ 7.54-7.17 (m, 5H), 5.21-4.90 (m, 2H), 3.63-3.45 (m, 2H), 3.17 (br d, J=5.0 Hz, 1H), 2.97 (br s, 1H), 1.79 (br s, 1H), 1.28 (br s, 1H), 0.61 (br s, 1H), 0.25 (br s, 1H), 0.26 (br s, 1H).

### Benzyl 6-(6-((4-cyano-2-fluorobenzyl)oxy)pyridin-2-yl)-3-azabicyclo[4.1.0]heptanes-3-carboxylate (9e)

To a solution of 4-(((6-bromopyridin-2-yl)oxy)methyl)-3-fluorobenzonitrile (9d, 200 mg, 651.22 umol, 1.1 eq) and [(Z)-(3-benzyloxycarbonyl-3-azabicyclo[4.1.0]heptan-6-yl)-boranylidene-fluoranyl]-difluoro-potassium (9c, 199.62 mg, 592.02 umol, 1 eq) in H2O (0.5 mL) and toluene (5 mL) was added Cs2CO3 (578.67 mg, 1.78 mmol, 3 eq), CatacXium A Pd G3 (21.56 mg, 29.60 umol, 0.05 eq) at 25° C. under N2. The mixture was stirred at 80° C. for 16 hours under N2. LCMS showed 9d was consumed, and desired MS was detected. The mixture was filtered and the filtrate was concentrated under reduced pressure. The residue was purified by column chromatography (SiO2, Petroleum ether:Ethyl acetate=10:1 to 0:1) to give 9e as yellow oil. 1H NMR (400 MHz, CDCl3-d) δ 7.61-7.49 (m, 2H), 7.45-7.29 (m, 7H), 6.89-6.73 (m, 1H), 6.60 (d, J=8.2 Hz, 1H), 5.43 (br s, 2H), 5.13 (s, 2H), 3.87-3.72 (m, 2H), 3.59 (br s, 1H), 3.28 (br s, 1H), 2.51-2.39 (m, 1H), 2.10 (br s, 1H), 1.74 (br d, J=15.8 Hz, 1H), 1.24 (br s, 1H), 0.92 (t, J=5.2 Hz, 1H).

### 4-(((6-(3-azabicyclo[4.1.0]heptan-6-yl)pyridin-2-yl)oxy)methyl)-3-fluorobenzonitrile (9f)

To a solution of Et3SiH (50.83 mg, 437.16 umol, 69.82 uL, 2.5 eq), TEA (8.85 mg, 87.43 umol, 12.17 uL, 0.5 eq) and Pd(OAc)2 (3.93 mg, 17.49 umol, 0.1 eq) in EtOH (2 mL) was added benzyl 6-(6-((4-cyano-2-fluorobenzyl)oxy)pyridin-2-yl)-3-azabicyclo[4.1.0] heptane-3-carboxylate (9e, 80 mg, 174.87 umol, 1 eq) at 20° C. The mixture was stirred at 20° C. for 16 hours under N2. TLC (Petroleum ether:Ethyl acetate=5:1) showed 9e was consumed, and one new spot was formed. The mixture was filtered and the filtrate was concentrated in vacuo. The residue was purified by prep-TLC (Petroleum ether:Ethyl acetate=5:1) to give 9f as colourless oil.

### Methyl 2-((6-(6-((4-cyano-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylate (9g)

K2CO3 (55.56 mg, 402.02 umol, 5 eq) was added to the solution of 4-(((6-(3-azabicyclo[4.1.0]heptan-6-yl)pyridin-2-yl)oxy)methyl)-3-fluorobenzonitrile (9f, 26 mg, 80.40 umol, 1 eq) and (S)-methyl 2-(chloromethyl)-1-(oxetan-2-ylmethyl)-1H-benzo[d]imidazole-6-carboxylate (1k, 23.70 mg, 80.40 umol, 1 eq) in CH3CN (1.5 mL) at 20° C. Then the solution was stirred at 50° C. for 2 hours. TLC (Petroleum ether:Ethyl acetate=0:1) showed 9f was consumed, and one new major spot was formed. The mixture was concentrated under reduced pressure. The residue was purified by prep-TLC (Petroleum ether:Ethyl acetate=0:1) to give 9g as a colourless oil. 1H NMR (400 MHz, MeOD-d4) δ 8.32 (s, 1H), 7.95 (d, J=7.8 Hz, 1H), 7.67 (dd, J=2.8, 8.6 Hz, 1H), 7.62-7.47 (m, 4H), 6.90 (d, J=7.6 Hz, 1H), 6.60 (d, J=8.2 Hz, 1H), 5.51-5.37 (m, 2H), 5.25-5.15 (m, 1H), 4.87-4.81 (m, 1H), 4.68 (dd, J=2.4, 15.4 Hz, 1H), 4.65-4.53 (m, 1H), 4.49-4.33 (m, 1H), 3.99-3.74 (m, 5H), 2.95-2.67 (m, 3H), 2.59-2.32 (m, 4H), 2.08-1.98 (m, 1H), 1.77-1.65 (m, 1H), 1.16-1.08 (m, 1H), 0.97-0.88 (m, 1H).

### 2-((6-(6-((4-cyano-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylic acid (Compound 9)

LiOH·H2O (793.62 ug, 18.91 umol, 1.1 eq) was added to the solution of methyl 2-((6-(6-((4-cyano-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylate (9g, 10 mg, 17.19 umol, 1 eq) in THF (0.7 mL) and H2O (0.3 mL) at 20° C. Then the solution was stirred at 20° C. for 20 hours. LCMS showed 9g was consumed, and desired MS was detected. The mixture was adjusted pH=6 with HOAC, and concentrated in vacuo. The residue was purified by prep-TLC (Dichloromethane:Methanol=10:1) to give Compound 9 as white solid. MS mass calculated for [M+1]+(C32H30FN5O4) requires m/z 568.2, LCMS found m/z 568.3. 1H NMR (400 MHz, MeOD-d4) δ 8.31 (s, 1H), 7.97 (d, J=8.6 Hz, 1H), 7.71-7.46 (m, 5H), 6.91 (d, J=7.6 Hz, 1H), 6.61 (d, J=8.1 Hz, 1H), 5.50-5.38 (m, 2H), 5.25-5.16 (m, 1H), 4.72-4.64 (m, 1H), 4.63-4.55 (m, 1H), 4.43 (tdd, J=5.8, 9.2, 18.4 Hz, 1H), 4.06-3.81 (m, 2H), 3.04-2.90 (m, 1H), 2.88-2.68 (m, 2H), 2.62-2.53 (m, 1H), 2.52-2.41 (m, 3H), 2.12-2.00 (m, 1H), 1.80-1.67 (m, 1H), 1.44-1.23 (m, 1H), 1.14 (td, J=3.4, 9.0 Hz, 1H), 1.00-0.92 (m, 1H);

### 2-(((1R,6S)-6-(6-((4-cyano-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylic acid (Compound 9-P1) & 2-(((1S,6R)-6-(6-((4-cyano-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylic acid (Compound 9-P2)

2-((6-(6-((4-cyano-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylic acid (Compound 9) was separated by Chiral SFC (DAICEL CHIRALPAK IG (250 mm*30 mm, 10 um); mobile phase: [0.1% NH3H2O MEOH]; B %: 60%-60%, min) to give Compound 9-P1 as white solid. MS mass calculated for [M+1]+(C32H30FN5O4) requires m/z 568.2, LCMS found m/z 568.3. 1H NMR (400 MHz, MeOD-d4) δ 8.31 (d, J=0.8 Hz, 1H), 7.97 (dd, J=1.4, 8.5 Hz, 1H), 7.70-7.49 (m, 5H), 6.92 (d, J=7.6 Hz, 1H), 6.62 (d, J=8.2 Hz, 1H), 5.51-5.40 (m, 2H), 5.21 (br dd, J=2.2, 7.4 Hz, 1H), 4.87-4.80 (m, 1H), 4.69 (dd, J=2.4, 15.3 Hz, 1H), 4.63-4.55 (m, 1H), 4.45 (td, J=6.0, 9.1 Hz, 1H), 4.01 (s, 1H), 3.87 (d, J=13.8 Hz, 1H), 2.98-2.90 (m, 1H), 2.88-2.82 (m, 1H), 2.80-2.69 (m, 1H), 2.63-2.42 (m, 4H), 2.11-2.01 (m, 1H), 1.81-1.69 (m, 1H), 1.19-1.12 (m, 1H), 0.96 (dd, J=3.8, 6.0 Hz, 1H).

Compound 9-P2 was obtained as white solid. MS mass calculated for [M+1]+(C32H30FN5O4) requires m/z 568.2, LCMS found m/z 568.3. 1H NMR (400 MHz, MeOD-d4) δ 8.31 (s, 1H), 7.97 (d, J=8.4 Hz, 1H), 7.70-7.48 (m, 5H), 6.92 (d, J=7.6 Hz, 1H), 6.62 (d, J=8.2 Hz, 1H), 5.46 (d, J=2.6 Hz, 2H), 5.26-5.15 (m, 1H), 4.84 (br d, J=7.0 Hz, 1H), 4.69 (dd, J=2.4, 15.4 Hz, 1H), 4.63-4.54 (m, 1H), 4.41 (td, J=5.8, 9.1 Hz, 1H), 3.94 (q, J=13.8 Hz, 2H), 3.04-2.93 (m, 1H), 2.82-2.67 (m, 2H), 2.62-2.52 (m, 1H), 2.52-2.41 (m, 3H), 2.13-2.01 (m, 1H), 1.80-1.69 (m, 1H), 1.19-1.10 (m, 1H), 0.95 (dd, J=3.8, 5.9 Hz, 1H).

When a mixture of stereoisomers is separated by HPLC, it is to be appreciated that the resultant individual stereoisomers or mixtures will be arbitrarily assigned. In the examples described herein, when the mixture of stereoisomers is separated by HPLC, it is to be appreciated that an eluting enantiomer or an enantiomer of a resulting compound prepared from the eluting enantiomer is labeled “P1” and another eluting enantiomer or an enantiomer of a resulting compound prepared from the another eluting enantiomer is labeled “P2”. In this example, the eluting enantiomers are of Compound 9. The absolute configuration of the enantiomers, e.g., Compounds 9-P1 & 9-P2 each associated with the corresponding 1H NMR data, may be obtained by known methods.

### Example 10 (General Procedure J)

### 2-(((1R,6S)-6-(6-(benzyloxy)pyridin-2-yl)-3-azabicyclo[4.1.0]heptan-3-yl)methyl)-1-((S)-oxetan-2-ylmethyl)-1H-benzo[d]imidazole-6-carboxylic acid

### and

### 2-(((1S,6R)-6-(6-(benzyloxy)pyridin-2-yl)-3-azabicyclo[4.1.0]heptan-3-yl)methyl)-1-((S)-oxetan-2-ylmethyl)-1H-benzo[d]imidazole-6-carboxylic acid

The title compounds were prepared according to Scheme 7. This General Procedure J exemplifies Scheme 7 and provides particular synthetic details as applied to the title compounds.

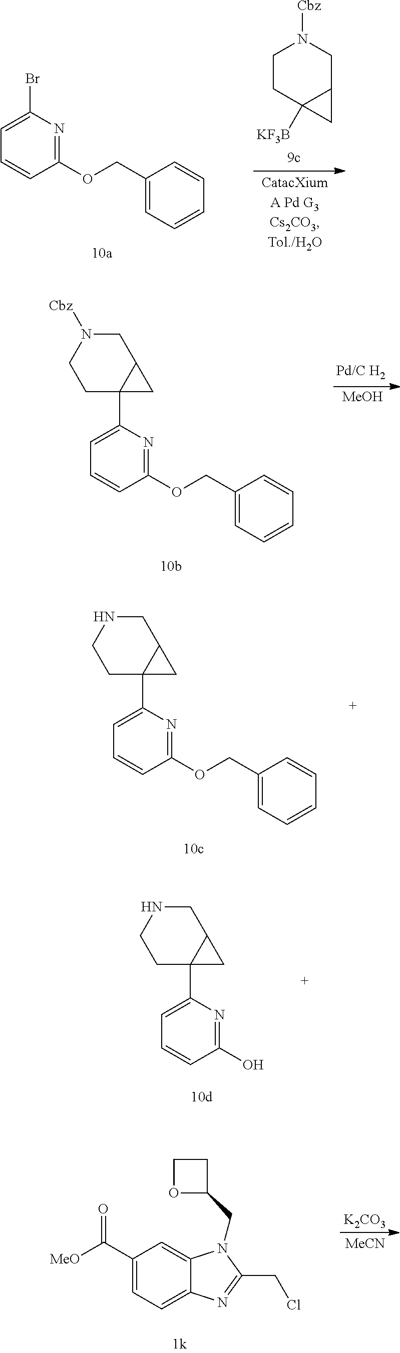

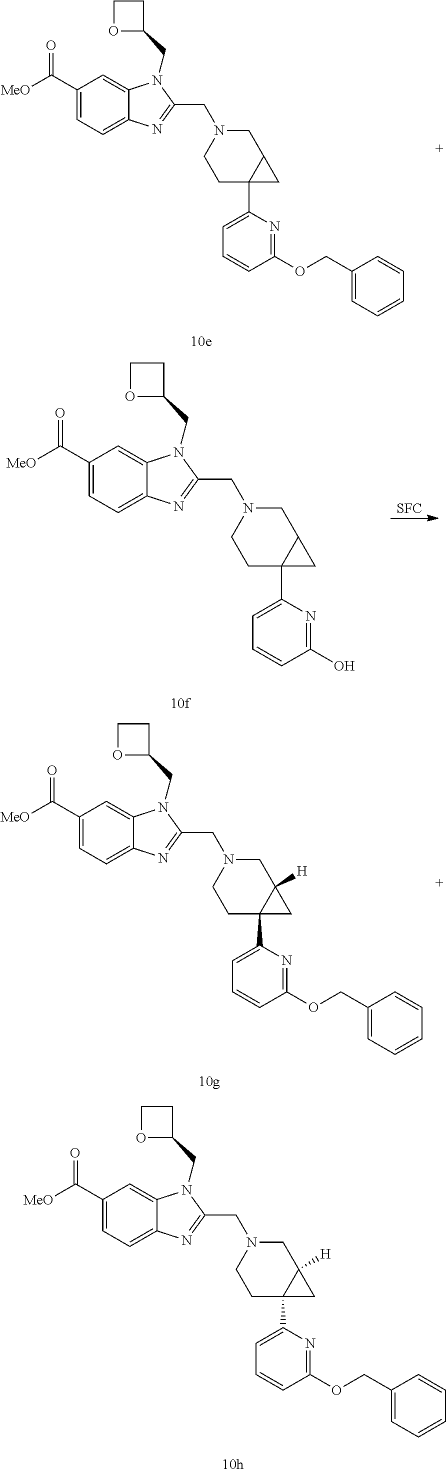 [SMILES: COC(=O)c1ccc2nc(CN3CCC4(c5cccc(OCc6ccccc6)n5)CC4C3)n(C[C@@H]3CCO3)c2c1 ; COC(=O)c1ccc2nc(CN3CCC4(c5cccc(O)n5)CC4C3)n(C[C@@H]3CCO3)c2c1 ; "SFC" ; COC(=O)c1ccc2nc(CN3CC[C@@]4(c5cccc(OCc6ccccc6)n5)C[C@H]4C3)n(C[C@@H]3CCO3)c2c1 ; COC(=O)c1ccc2nc(CN3CC[C@]4(c5cccc(OCc6ccccc6)n5)C[C@@H]4C3)n(C[C@@H]3CCO3)c2c1]

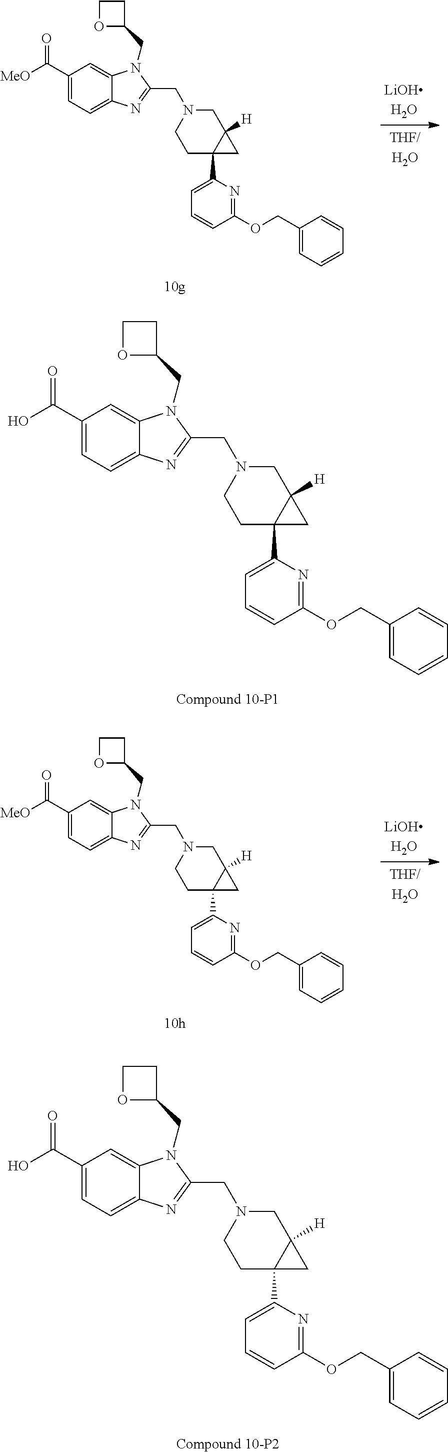 [SMILES: COC(=O)c1ccc2nc(CN3CC[C@@]4(c5cccc(OCc6ccccc6)n5)C[C@H]4C3)n(C[C@@H]3CCO3)c2c1 ; O=C(O)c1ccc2nc(CN3CC[C@@]4(c5cccc(OCc6ccccc6)n5)C[C@H]4C3)n(C[C@@H]3CCO3)c2c1 ; COC(=O)c1ccc2nc(CN3CC[C@]4(c5cccc(OCc6ccccc6)n5)C[C@@H]4C3)n(C[C@@H]3CCO3)c2c1 ; O=C(O)c1ccc2nc(CN3CC[C@]4(c5cccc(OCc6ccccc6)n5)C[C@@H]4C3)n(C[C@@H]3CCO3)c2c1]

Benzyl 6-(6-(benzyloxy)pyridin-2-yl)-3-azabicyclo[4.1.0]heptane-3-carboxylate (10b). 2-(benzyloxy)-6-bromopyridine (10a, 13.79 mg, 18.93 umol, 0.05 eq) and Cs2CO3 (370.08 mg, 1.14 mmol, 3 eq) was added to the solution of 2-benzyloxy-6-bromo-pyridine (0.1 g, 378.62 umol, 1 eq) and [(Z)-(3-benzyloxycarbonyl-3-azabicyclo[4.1.0]heptan-6-yl) boranylidene-fluoranyl]-difluoro-potassium (9c, 140.43 mg, 416.48 umol, 1.1 eq) in toluene (2 mL) and H2O (0.2 mL) at 20° C. Then the reaction was stirred at 80° C. for 16 hours under N2. TLC (Petroleum ether:Ethyl acetate=5:1) showed 10a was consumed, and one major new spot was formed. The mixture was filtered and the filtrate was concentrated in vacuo. The residue was purified by column chromatography (SiO2, Petroleum ether:Ethyl acetate=80:1 to 20:1) to give 10b as a light yellow oil. 1H NMR (400 MHz, MeOD-d4) δ 7.54 (t, J=7.8 Hz, 1H), 7.42-7.31 (m, 7H), 7.26 (br d, J=7.2 Hz, 1H), 7.30 (s, 1H), 6.85 (br d, J=7.2 Hz, 1H), 6.58 (d, J=8.2 Hz, 1H), 5.32 (d, J=1.6 Hz, 2H), 5.12 (s, 2H), 3.89-3.67 (m, 2H), 3.51 (td, J=5.8, 13.4 Hz, 1H), 3.35 (s, 1H), 2.50 (ddd, J=5.8, 8.4, 13.8 Hz, 1H), 2.05 (br d, J=12.6 Hz, 1H), 1.73 (dtd, J=2.6, 5.6, 8.4 Hz, 1H), 1.30 (br d, J=8.6 Hz, 1H), 0.89 (t, J=5.2 Hz, 1H).

### 6-(6-(benzyloxy)pyridin-2-yl)-3-azabicyclo[4.1.0]heptane (10c) & 6-(3-azabicyclo[4.1.0]heptan-6-yl)pyridin-2-ol (10d)

Benzyl 6-(6-(benzyloxy)pyridin-2-yl)-3-azabicyclo[4.1.0]heptane-3-carboxylate (10b, 400 mg, 965.03 umol, 1 eq) was added to the solution of Pd/C (200 mg, 965.03 umol, 90% purity, 1 eq) in MeOH (15 mL) at 20° C. Then the solution was stirred at 20° C. for 0.5 hour under H2 (15Psi). LCMS detected the desired product MS and showed that the reaction was not complete. Then the solution was stirred at 20° C. for 3.5 hours under H2 (15Psi). LCMS detected the desired product MS and showed that the reaction was complete. The mixture was filtered and the filtrate concentrated to give a mixture of 10c and 10d as light yellow oil. The products mixture was used directly in next step without any further purification.

### Methyl 2-((6-(6-(benzyloxy)pyridin-2-yl)-3-azabicyclo[4.1.0]heptan-3-yl)methyl)-1-((S)-oxetan-2-ylmethyl)-1H-benzo[d]imidazole-6-carboxylate (10e) and methyl 2-((6-(6-hydroxypyridin-2-yl)-3-azabicyclo[4.1.0]heptan-3-yl)methyl)-1-((S)-oxetan-2-ylmethyl)-1H-benzo[d]imidazole-6-carboxylate (10f)

K2CO3 (295.78 mg, 2.14 mmol, 5 eq) was added to the solution of 6-(3-azabicyclo[4.1.0]heptan-6-yl)pyridin-2-ol (10d, 81.43 mg, 428.02 umol, 1 eq), 6-(6-(benzyloxy)pyridin-2-yl)-3-azabicyclo[4.1.0]heptane (10c, 120 mg, 428.02 umol, 1 eq) and (S)-methyl 2-(chloromethyl)-1-(oxetan-2-ylmethyl)-1H-benzo[d]imidazole-6-carboxylate (1k, 113.54 mg, 385.21 umol, 0.9 eq) in CH3CN (9 mL) at 20° C. Then the solution was stirred at 50° C. for 3 hours. LCMS showed 10c and 10d were consumed, and desired MS was detected. The mixture was concentrated to remove the solvent. The residue was purified by column chromatography (SiO2, Petroleum ether:Ethyl acetate=80:1 to 20:1) to give 10e as light yellow oil. 1H NMR (400 MHz, MeOD-d4) δ 8.34 (s, 1H), 8.00-7.92 (m, 1H), 7.72-7.67 (m, 1H), 7.54 (t, J=7.8 Hz, 1H), 7.42-7.37 (m, 2H), 7.35-7.28 (m, 2H), 7.26 (d, J=7.2 Hz, 1H), 6.88 (d, J=7.6 Hz, 1H), 6.56 (d, J=8.2 Hz, 1H), 5.32 (d, J=3.2 Hz, 2H), 5.22 (dt, J=2.6, 7.2 Hz, 1H), 4.85 (s, 1H), 4.71 (dd, J=2.2, 15.4 Hz, 1H), 4.60 (s, 1H), 4.52-4.35 (m, 1H), 4.02-3.90 (m, 4H), 3.89-3.76 (m, 1H), 3.03-2.69 (m, 2H), 2.63-2.37 (m, 4H), 2.21-1.90 (m, 1H), 1.86-1.77 (m, 1H), 1.49-1.45 (m, 1H), 1.23-1.18 (m, 1H), 0.99-0.91 (m, 1H).

10f was obtained as a light yellow solid. 1H NMR (400 MHz, MeOD-d4) δ 8.34 (s, 1H), 8.02-7.94 (m, 1H), 7.70 (dd, J=3.0, 8.5 Hz, 1H), 7.49 (dd, J=7.4, 8.6 Hz, 1H), 6.39-6.34 (m, 1H), 6.28 (d, J=7.2 Hz, 1H), 5.29-5.17 (m, 1H), 4.84 (br d, J=5.0 Hz, 1H), 4.74-4.58 (m, 2H), 4.51-4.37 (m, 1H), 4.05-3.78 (m, 5H), 3.05-2.95 (m, 1H), 2.84-2.69 (m, 2H), 2.57-2.34 (m, 3H), 2.21-2.11 (m, 1H), 2.02 (s, 1H), 1.61-1.50 (m, 1H), 1.08 (td, J=4.4, 9.1 Hz, 1H), 1.03-0.90 (m, 1H).

Methyl 2-(((1R,6S)-6-(6-(benzyloxy)pyridin-2-yl)-3-azabicyclo[4.1.0]heptan-3-yl)methyl)-1-((S)-oxetan-2-ylmethyl)-1H-benzo[d]imidazole-6-carboxylate (10g) and methyl 2-(((1S,6R)-6-(6-(benzyloxy)pyridin-2-yl)-3-azabicyclo[4.1.0]heptan-3-yl)methyl)-1-((S)-oxetan-2-ylmethyl)-1H-benzo[d]imidazole-6-carboxylate (10h). methyl 2-((6-(6-(benzyloxy)pyridin-2-yl)-3-azabicyclo[4.1.0]heptan-3-yl)methyl)-1-((S)-oxetan-2-ylmethyl)-1H-benzo[d]imidazole-6-carboxylate (10e, 70 mg, 129.96 umol, 1 eq) was separated by Chiral SFC (column: DAICEL CHIRALPAK AD (250 mm*30 mm, 10 um); mobile phase: % NH3H2O ETOH]; B %: 50%-50%, min) to give 10g as a white solid. 1H NMR (400 MHz, MeOD-d4) δ 8.33 (d, J=1.0 Hz, 1H), 7.96 (dd, J=1.4, 8.6 Hz, 1H), 7.67 (d, J=8.6 Hz, 1H), 7.53 (t, J=7.8 Hz, 1H), 7.41-7.35 (m, 2H), 7.31 (t, J=7.4 Hz, 3H), 6.87 (d, J=7.6 Hz, 1H), 6.55 (d, J=8.2 Hz, 1H), 5.37-5.26 (m, 2H), 5.20 (br dd, J=2.4, 7.3 Hz, 1H), 4.89-4.83 (m, 1H), 4.70 (dd, J=2.4, 15.3 Hz, 1H), 4.64-4.55 (m, 1H), 4.46 (td, J=6.0, 9.1 Hz, 1H), 4.04-3.89 (m, 4H), 3.79 (d, J=13.8 Hz, 1H), 2.92-2.68 (m, 3H), 2.63-2.43 (m, 2H), 2.40 (t, J=6.0 Hz, 2H), 2.06 (td, J=6.4, 13.4 Hz, 1H), 1.86-1.76 (m, 1H), 1.22 (dd, J=3.6, 9.1 Hz, 1H), 0.95 (dd, J=3.6, 6.0 Hz, 1H).

10h was obtained as a white solid. 1H NMR (400 MHz, MeOD-d4) δ 8.33 (d, J=1.0 Hz, 1H), 7.96 (dd, J=1.6, 8.6 Hz, 1H), 7.68 (d, J=8.6 Hz, 1H), 7.53 (t, J=7.8 Hz, 1H), 7.38 (br d, J=7.2 Hz, 2H), 7.34-7.20 (m, 3H), 6.87 (d, J=7.6 Hz, 1H), 6.55 (d, J=8.2 Hz, 1H), 5.36-5.26 (m, 2H), 5.25-5.17 (m, 1H), 4.86 (br d, J=7.0 Hz, 1H), 4.70 (dd, J=2.6, 15.4 Hz, 1H), 4.62-4.54 (m, 1H), 4.40 (td, J=6.0, 9.2 Hz, 1H), 3.97-3.89 (m, 4H), 3.88-3.82 (m, 1H), 2.98-2.90 (m, 1H), 2.78-2.66 (m, 2H), 2.62-2.53 (m, 1H), 2.51-2.37 (m, 3H), 2.12-2.01 (m, 1H), 1.86-1.75 (m, 1H), 1.22 (dd, J=3.6, 9.2 Hz, 1H), 0.93 (dd, J=3.8, 6.0 Hz, 1H).

### 2-(((1R,6S)-6-(6-(benzyloxy)pyridin-2-yl)-3-azabicyclo[4.1.0]heptan-3-yl)methyl)-1-((S)-oxetan-2-ylmethyl)-1H-benzo[d]imidazole-6- carboxylic acid (Compound 10-P1)

LiOH·H2O (6.31 mg, 150.38 umol, 3 eq) was added to the solution of methyl 2-(((1R,6S)-6-(6-(benzyloxy)pyridin-2-yl)-3-azabicyclo[4.1.0]heptan-3-yl)methyl)-1-((S)-oxetan-2-ylmethyl)-1H-benzo[d]imidazole-6-carboxylate (10g, 27 mg, 50.13 umol, 1 eq) in THF (2.1 mL) and H2O (0.9 mL) at 20° C. Then the solution was stirred at 20° C. for 24 hours. LCMS showed 10 g was consumed, and desired MS was detected. The mixture was adjusted to pH=7 with HOAc, and extracted with Ethyl acetate (10 mL*3). The combined organic layers were washed with brine (15 mL), dried over Na2SO4, filtered and concentrated in vacuo. The residue was purified by prep-HPLC (Neutral condition, column: Phenomenex Gemini-NX C18 75*30 mm*3 um; mobile phase: [water (10 mM NH4HCO3)-ACN]; B %: 30%-50%, 6 min) to give Compound 10-P1 as a white solid. MS mass calculated for [M+1]+(C31H32N4O4) requires m/z 525.2, LCMS found m/z 525.2; 1H NMR (400 MHz, MeOD-d4) δ 8.31 (s, 1H), 7.97 (dd, J=1.4, 8.5 Hz, 1H), 7.67 (d, J=8.6 Hz, 1H), 7.54 (t, J=7.8 Hz, 1H), 7.42-7.35 (m, 2H), 7.33-7.20 (m, 3H), 6.88 (d, J=7.4 Hz, 1H), 6.56 (d, J=8.2 Hz, 1H), 5.36-5.26 (m, 2H), 5.20 (br dd, J=2.2, 7.3 Hz, 1H), 4.85 (dd, J=7.4, 15.4 Hz, 1H), 4.68 (dd, J=2.4, 15.3 Hz, 1H), 4.59 (br d, J=6.2 Hz, 1H), 4.48-4.39 (m, 1H), 4.05 (d, J=13.8 Hz, 1H), 3.88 (d, J=13.6 Hz, 1H), 3.04-2.93 (m, 1H), 2.86 (br d, J=11.2 Hz, 1H), 2.79-2.68 (m, 1H), 2.66-2.56 (m, 1H), 2.55-2.41 (m, 3H), 2.15-2.02 (m, 1H), 1.84 (br d, J=7.4 Hz, 1H), 1.24 (dd, J=3.6, 9.1 Hz, 1H), 0.97 (dd, J=3.8, 5.9 Hz, 1H).

### 2-(((1S,6R)-6-(6-(benzyloxy)pyridin-2-yl)-3-azabicyclo[4.1.0]heptan-3-yl)methyl)-1-((S)-oxetan-2-ylmethyl)-1H-benzo[d]imidazole-6-carboxylic acid (Compound 10-P2)

LiOH·H2O (5.69 mg, 135.71 umol, 3 eq) was added to the solution of methyl 2-(((1S,6R)-6-(6-(benzyloxy)pyridin-2-yl)-3-azabicyclo[4.1.0]heptan-3-yl)methyl)-1-((S)-oxetan-2-ylmethyl)-1H-benzo[d]imidazole-6-carboxylate (10h, 25 mg, 45.24 umol, 1 eq) in THF (2.1 mL) and H2O (0.9 mL) at 20° C. Then the solution was stirred at 20° C. for 32 hours. LCMS showed 10h was consumed, and desired MS was detected. The mixture was adjusted to pH=7 with HOAc. The mixture was extracted with Ethyl acetate (10 mL*3). The combined Ethyl acetate was washed with brine (15 mL), dried over Na2SO4, filtered and concentrated in vacuo. The residue was purified by prep-HPLC (Neutral condition, column: Waters Xbridge BEH C18 100*25 mm*5 um; mobile phase: [water (10 mM NH4HCO3)-ACN]; B %: 35%-65%, 10 min) to give 10-P2 as a white solid. MS mass calculated for [M+1]+(C31H32N4O4) requires m/z 525.2, LCMS found m/z 525.2; 1H NMR (400 MHz, MeOD-d4) δ 8.27 (s, 1H), 7.96 (dd, J=1.4, 8.5 Hz, 1H), 7.65 (d, J=8.4 Hz, 1H), 7.53 (t, J=7.8 Hz, 1H), 7.42-7.35 (m, 2H), 7.31 (t, J=7.6 Hz, 3H), 6.87 (d, J=7.4 Hz, 1H), 6.55 (d, J=8.2 Hz, 1H), 5.32 (d, J=2.2 Hz, 2H), 5.26-5.18 (m, 1H), 4.86-4.81 (m, 1H), 4.71 (s, 1H), 4.63-4.53 (m, 1H), 4.46-4.36 (m, 1H), 3.92 (q, J=13.8 Hz, 2H), 2.97 (dd, J=6.4, 11.3 Hz, 1H), 2.82-2.67 (m, 2H), 2.64-2.54 (m, 1H), 2.44 (s, 3H), 2.13-2.04 (m, 1H), 1.86-1.76 (m, 1H), 1.24 (s, 1H), 0.95 (dd, J=3.8, 5.8 Hz, 1H).

When a mixture of stereoisomers is separated by HPLC, it is to be appreciated that the resultant individual stereoisomers or mixtures will be arbitrarily assigned. In the examples described herein, when the mixture of stereoisomers is separated by HPLC, it is to be appreciated that an eluting enantiomer or an enantiomer of a resulting compound prepared from the eluting enantiomer is labeled “P1” and another eluting enantiomer or an enantiomer of a resulting compound prepared from the another eluting enantiomer is labeled “P2”. In this example, the eluting enantiomers are 10g and 10h, and the resulting compound is Compound 10. The absolute configuration of the enantiomers, e.g., 10g & 10h, as well as Compounds 10-P1 & 10-P2 each associated with the corresponding 1H NMR data, may be obtained by known methods.

### Example 11 (General Procedure K)

### 2-(((1R,6S)-6-(6-((4-chloro-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylic acid

### and

### 2-(((1S,6R)-6-(6-((4-chloro-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylic acid

The title compound was prepared according to Scheme 8. This General Procedure K exemplifies Scheme 8 and provides particular synthetic details as applied to the title compound.

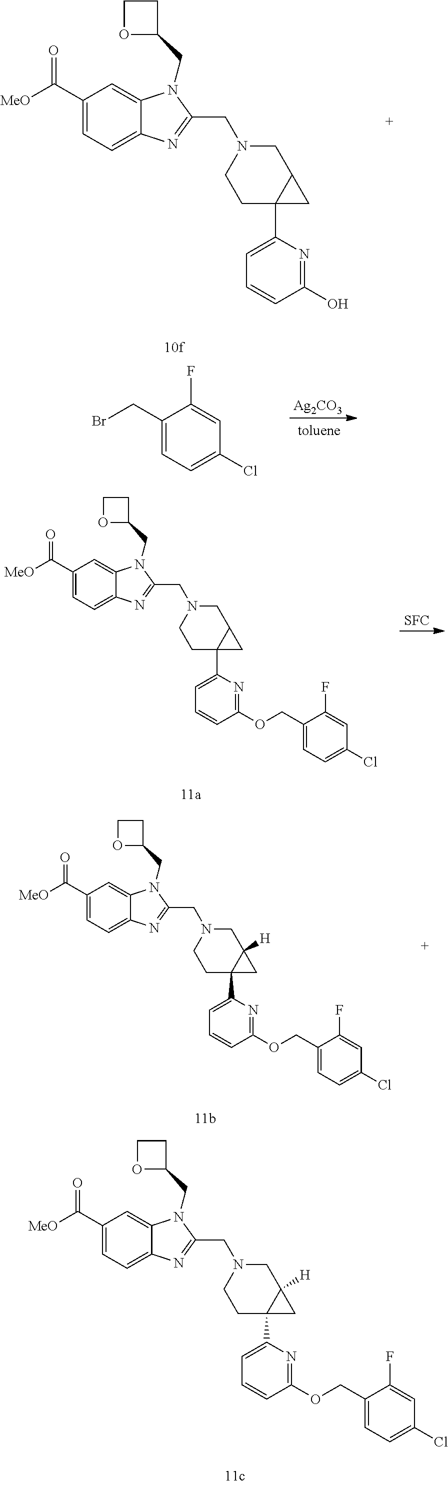

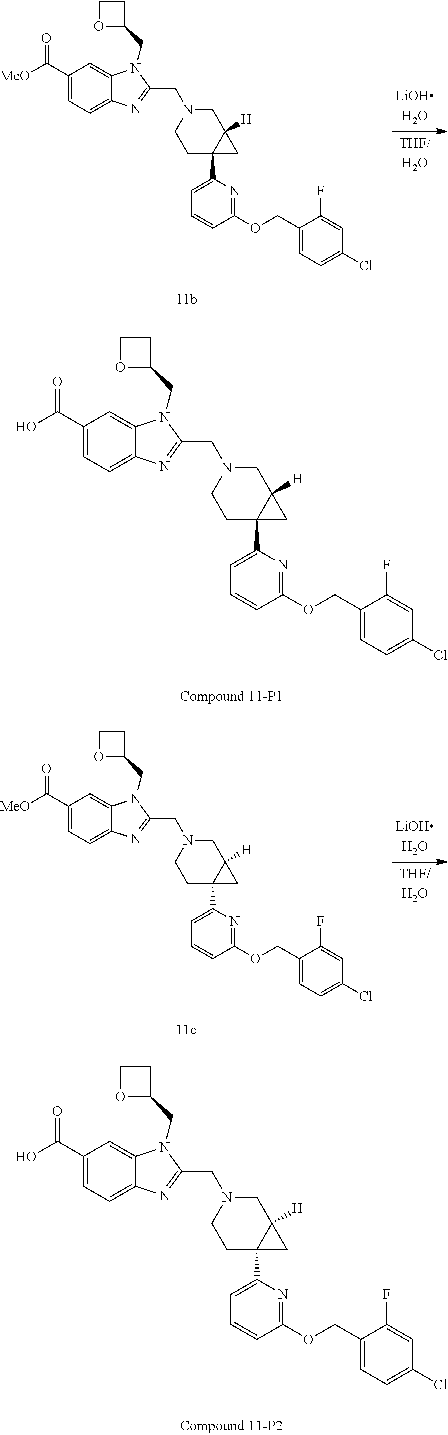 [SMILES: COC(=O)c1ccc2nc(CN3CC[C@@]4(c5cccc(OCc6ccc(Cl)cc6F)n5)C[C@H]4C3)n(C[C@@H]3CCO3)c2c1 ; O=C(O)c1ccc2nc(CN3CC[C@@]4(c5cccc(OCc6ccc(Cl)cc6F)n5)C[C@H]4C3)n(C[C@@H]3CCO3)c2c1 ; COC(=O)c1ccc2nc(CN3CC[C@]4(c5cccc(OCc6ccc(Cl)cc6F)n5)C[C@@H]4C3)n(C[C@@H]3CCO3)c2c1 ; O=C(O)c1ccc2nc(CN3CC[C@]4(c5cccc(OCc6ccc(Cl)cc6F)n5)C[C@@H]4C3)n(C[C@@H]3CCO3)c2c1]

### Methyl 2-((6-(6-((4-chloro-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylate (11a)

To the solution of methyl 2-((6-(6-hydroxypyridin-2-yl)-3-azabicyclo[4.1.0]heptan-3-yl)methyl)-1-((S)-oxetan-2-ylmethyl)-1H-benzo[d]imidazole-6-carboxylate (10f, 90 mg, 200.66 umol, 1 eq) and 1-(bromomethyl)-4-chloro-2-fluorobenzene (62.78 mg, 280.93 umol, 1.4 eq) in toluene (5 mL) was added Ag2CO3 (110.66 mg, 401.33 umol, 18.20 uL, 2 eq) at 20° C. Then the solution was stirred at 100° C. for 3 hours. TLC (Ethyl acetate:Methanol=10:1) showed 10f was disappeared and one new spot was formed. The solution was filtered and the filtrate concentrated in vacuo. The residue was purified by column chromatography (SiO2, Petroleum ether:Ethyl acetate=80:1 to 0:1) to give 11a as a light yellow solid. 1H NMR (400 MHz, MeOD-d4) δ 8.33 (s, 1H), 7.96 (td, J=1.6, 8.6 Hz, 1H), 7.68 (dd, J=2.8, 8.4 Hz, 1H), 7.55 (t, J=7.8 Hz, 1H), 7.43 (t, J=8.2 Hz, 1H), 7.48-7.39 (m, 1H), 6.89 (d, J=7.6 Hz, 1H), 6.56 (d, J=8.2 Hz, 1H), 5.36 (br s, 2H), 5.27-5.15 (m, 1H), 4.89 (br d, J=7.4 Hz, 1H), 4.74-4.66 (m, 1H), 4.64-4.55 (m, 1H), 4.50-4.37 (m, 1H), 4.03-3.89 (m, 4H), 3.89-3.76 (m, 1H), 3.00-2.68 (m, 3H), 2.63-2.34 (m, 4H), 2.12-2.01 (m, 1H), 1.78 (br d, J=3.6 Hz, 1H), 1.25-1.16 (m, 1H), 1.01-0.91 (m, 1H).

Methyl 2-(((1R,6S)-6-(6-((4-chloro-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylate (11b) and methyl 2-(((1S,6R)-6-(6-((4-chloro-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylate (11c). methyl 2-((6-(6-((4-chloro-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylate (11a, 80 mg, 135.35 umol, 1 eq) was separated by Chiral SFC (column: DAICEL CHIRALPAK AD (250 mm*30 mm, 10 um); mobile phase: [0.1% NH3H2O MEOH]; B %: 60%-60%, min) to give 11b as white solid. 1H NMR (400 MHz, MeOD-d4) δ 8.33 (s, 1H), 7.95 (dd, J=1.2, 8.4 Hz, 1H), 7.67 (d, J=8.4 Hz, 1H), 7.54 (t, J=7.8 Hz, 1H), 7.43 (t, J=8.2 Hz, 1H), 7.31-7.12 (m, 2H), 6.89 (d, J=7.6 Hz, 1H), 6.56 (d, J=8.2 Hz, 1H), 5.43-5.28 (m, 2H), 5.20 (dt, J=5.2, 7.2 Hz, 1H), 4.69 (dd, J=2.4, 15.2 Hz, 1H), 4.64-4.54 (m, 1H), 4.46 (td, J=6.0, 9.1 Hz, 1H), 4.02-3.89 (m, 4H), 3.81 (s, 1H), 2.93-2.69 (m, 3H), 2.62-2.35 (m, 5H), 2.12-1.99 (m, 1H), 1.83-1.72 (m, 1H), 1.20 (br d, J=5.4 Hz, 1H), 0.95 (dd, J=3.8, 5.9 Hz, 1H).

11c was obtained as a white solid. 1H NMR (400 MHz, MeOD-d4) δ 8.33 (d, J=0.8 Hz, 1H), 7.96 (dd, J=1.4, 8.6 Hz, 1H), 7.68 (d, J=8.4 Hz, 1H), 7.55 (t, J=7.8 Hz, 1H), 7.43 (t, J=8.2 Hz, 1H), 7.25-7.11 (m, 2H), 6.89 (d, J=7.4 Hz, 1H), 6.56 (d, J=8.2 Hz, 1H), 5.42-5.30 (m, 2H), 5.26-5.17 (m, 1H), 4.90 (br s, 1H), 4.70 (dd, J=2.6, 15.4 Hz, 1H), 4.59 (br d, J=6.2 Hz, 1H), 4.45-4.34 (m, 1H), 3.99-3.80 (m, 5H), 2.94 (br dd, J=6.2, 11.2 Hz, 1H), 2.80-2.65 (m, 2H), 2.63-2.52 (m, 1H), 2.51-2.36 (m, 3H), 2.14-1.99 (m, 1H), 1.83-1.73 (m, 1H), 1.20 (dd, J=3.6, 9.1 Hz, 1H), 0.95 (br dd, J=3.8, 5.9 Hz, 1H).

### 2-(((1R,6S)-6-(6-((4-chloro-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylic acid (11-P1)

LiOH·H2O (6.18 mg, 147.19 umol, 3 eq) was added to the solution of methyl 2-(((1R,6S)-6-(6-((4-chloro-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylate (11b, 29 mg, 49.06 umol, 1 eq) in THF (2.1 mL) and H2O (0.9 mL) at 20° C. Then the solution was stirred at 20° C. for 24 hours. LCMS showed 11b was consumed, and desired mass was detected. The mixture was adjusted to pH=7 with HOAc. The mixture was extracted with Ethyl acetate (10 mL*3). The combined organic layers were washed with brine (15 mL), dried over Na2SO4, filtered and concentrated. The residue was purified by prep-HPLC (Neutral condition, column: Waters Xbridge BEH) to give 11-P1 as a white solid. MS mass calculated for [M+1]+(C31H30C1FN4O4) requires m/z 577.2, LCMS found m/z 577.1; 1H NMR (400 MHz, MeOD-d4) δ 8.29 (s, 1H), 7.97 (dd, J=1.4, 8.5 Hz, 1H), 7.65 (d, J=8.6 Hz, 1H), 7.56 (t, J=7.8 Hz, 1H), 7.44 (t, J=8.2 Hz, 1H), 7.24-7.13 (m, 2H), 6.91 (d, J=7.6 Hz, 1H), 6.57 (d, J=8.2 Hz, 1H), 5.43-5.32 (m, 2H), 5.28-5.17 (m, 1H), 4.89-4.83 (m, 1H), 4.71 (dd, J=2.6, 15.4 Hz, 1H), 4.65-4.56 (m, 1H), 4.47 (td, J=5.8, 9.2 Hz, 1H), 4.01 (d, J=13.8 Hz, 1H), 3.84 (d, J=13.8 Hz, 1H), 2.97-2.90 (m, 1H), 2.87-2.81 (m, 1H), 2.80-2.70 (m, 1H), 2.64-2.39 (m, 4H), 2.14-2.03 (m, 1H), 1.87-1.75 (m, 1H), 1.22 (dd, J=3.6, 9.2 Hz, 1H), 0.98 (dd, J=3.8, 5.8 Hz, 1H).

### 2-(((1S,6R)-6-(6-((4-chloro-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylic acid (Compound 11-P2)

LiOH·H2O (8.52 mg, 203.02 umol, 3 eq) was added to the solution of methyl 2-(((1S,6R)-6-(6-((4-chloro-2-fluorobenzyl)oxy)pyridin-2-yl)-3-azabicyclo[4.1.0]heptan-3-yl)methyl)-1-((S)-oxetan-2-ylmethyl)-1H-benzo[d]imidazole-6-carboxylate (11c, 40 mg, 67.67 umol, 1 eq) in THF (2.8 mL) and H2O (1.2 mL) at 20° C. Then the solution was stirred at 20° C. for 24 hours. LCMS showed 11c was consumed, and desired mass was detected. The mixture was adjusted to pH=7 with HOAc. The mixture was extracted with Ethyl acetate (10 mL*3). The combined organic layers were washed with brine (15 mL), dried over Na2SO4, filtered and concentrated. The residue was purified by prep-HPLC (Neutral condition, column: Phenomenex Gemini-NX C18 75*30 mm*3 um; mobile phase: [water (10 mM NH4HCO3)-ACN]; B %: 30%-50%, 6 min) to give Compound 11-P2 as white solid. MS mass calculated for [M+1]+(C31H30ClFN4O4) requires m/z 577.2, LCMS found m/z 577.1; 1H NMR (400 MHz, MeOD-d4) δ 8.28 (s, 1H), 7.97 (br d, J=8.6 Hz, 1H), 7.65 (br d, J=8.4 Hz, 1H), 7.55 (t, J=7.8 Hz, 1H), 7.44 (t, J=8.0 Hz, 1H), 7.24-7.10 (m, 2H), 6.90 (d, J=7.6 Hz, 1H), 6.56 (d, J=8.2 Hz, 1H), 5.36 (s, 2H), 5.28-5.17 (m, 1H), 4.86 (br s, 1H), 4.75-4.64 (m, 1H), 4.59 (br d, J=6.4 Hz, 1H), 4.41 (br d, J=9.2 Hz, 1H), 3.92 (q, J=13.8 Hz, 2H), 3.03-2.90 (m, 1H), 2.82-2.68 (m, 2H), 2.58 (br dd, J=6.2, 13.1 Hz, 1H), 2.52-2.38 (m, 3H), 2.15-2.02 (m, 1H), 1.79 (br d, J=7.6 Hz, 1H), 1.20 (br dd, J=3.6, 8.9 Hz, 1H), 1.00-0.90 (m, 1H).

When a mixture of stereoisomers is separated by HPLC, it is to be appreciated that the resultant individual stereoisomers or mixtures will be arbitrarily assigned. In the examples described herein, when the mixture of stereoisomers is separated by HPLC, it is to be appreciated that an eluting enantiomer or an enantiomer of a resulting compound prepared from the eluting enantiomer is labeled “P1” and another eluting enantiomer or an enantiomer of a resulting compound prepared from the another eluting enantiomer is labeled “P2”. In this example, the eluting enantiomers are 11b and 11c, and the resulting compound is Compound 11. The absolute configuration of the enantiomers, e.g., 11b & 11c, as well as Compounds 11-P1 & 11-P2 each associated with the corresponding 1H NMR data, may be obtained by known methods.

### Example 12 (General Procedure L)

### 1-((S)-oxetan-2-ylmethyl)-2-((4-(6-(1,2,3,4-tetrahydronaphthalen-2-yl)pyridin-2-yl)piperidin-1-yl)methyl)-1H-benzo[d]imidazole-6-carboxylic acid

The title compound was prepared according to Scheme 9. This General Procedure L exemplifies Scheme 9 and provides particular synthetic details as applied to the title compound.

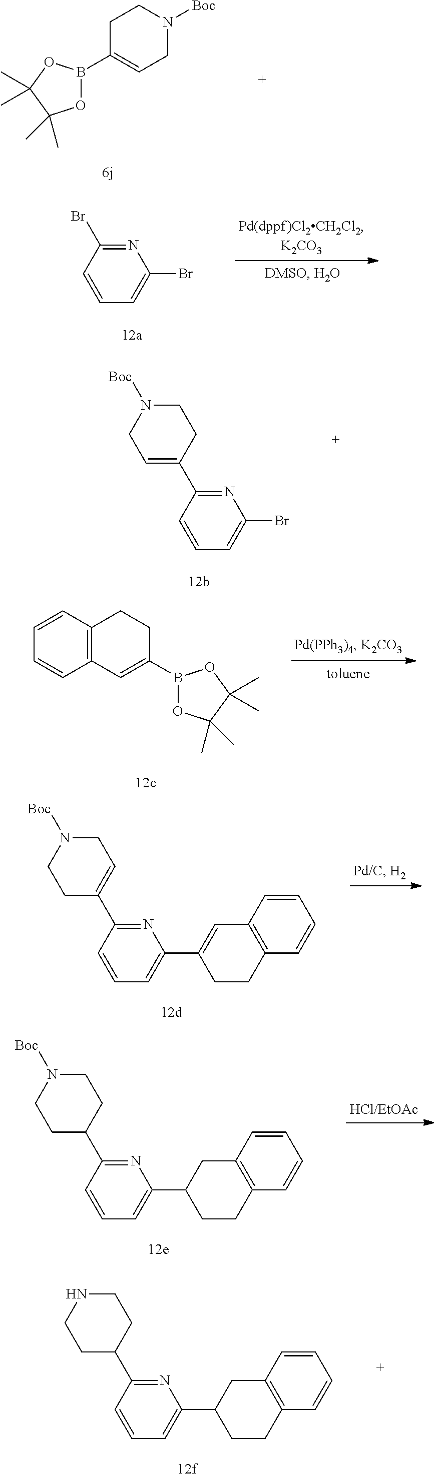

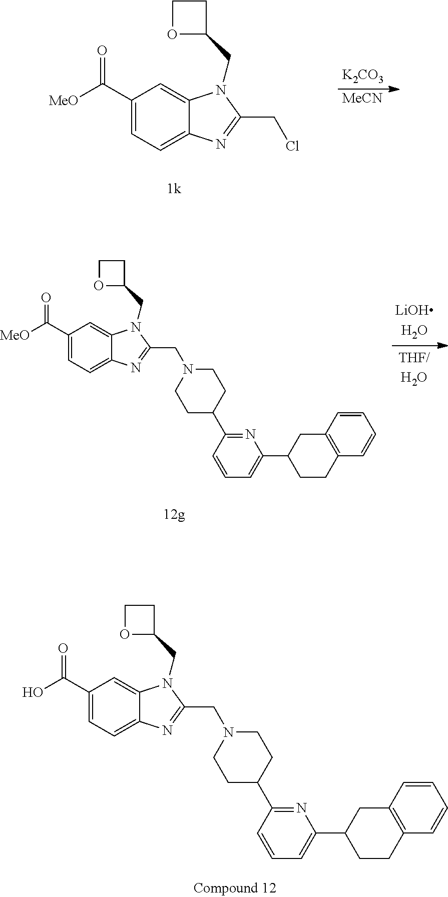 [SMILES: "MeCN" ; COC(=O)c1ccc2nc(CCl)n(C[C@@H]3CCO3)c2c1 ; "K2CO3" ; COC(=O)c1ccc2nc(CN3CCC(c4cccc(C5CCc6ccccc6C5)n4)CC3)n(C[C@@H]3CCO3)c2c1 ; O=C(O)c1ccc2nc(CN3CCC(c4cccc(C5CCc6ccccc6C5)n4)CC3)n(C[C@@H]3CCO3)c2c1]

### Tert-butyl 6-bromo-5′,6′-dihydro-[2,4′-bipyridine]-1′(2′H)-carboxylate (12b)

A mixture of tert-butyl 4-(4,4,5,5-tetramethyl-1,3,2-dioxaborolan-2-yl)-5,6-dihydropyridine-1(2H)-carboxylate (6j, 1g, 3.23 mmol, 1 eq), 2,6-dibromopyridine (12a, 919.35 mg, 3.88 mmol, 1.2 eq), Pd(dppf)Cl₂·CH2Cl₂ (132.05 mg, 161.70 umol, 0.05 eq), K2CO3 (1.34 g, 9.70 mmol, 3 eq) and in DMSO (15 mL) and H2O (1.5 mL) was degassed and purged with N2 3 times, and then the mixture was stirred at 80° C. for 3 hours under N2 atmosphere. LCMS showed 6j was consumed completely and desired mass was detected. The aqueous phase was extracted with ethyl acetate (30 mL*3). The combined organic phase was washed with brine (25 mL), dried with anhydrous Na2SO4, filtered and concentrated in vacuo. The crude product was purified by column chromatography (SiO2, Petroleum ether:Ethyl acetate=40:1 to 4:1) to give 12b as a white solid.

### Tert-butyl 6-(3,4-dihydronaphthalen-2-yl)-5′,6′-dihydro-[2,4′-bipyridine]-1′(2′H)-carboxylate (12d)

A mixture of tert-butyl 6-bromo-5′,6′-dihydro-[2,4′-bipyridine]-1′(2′H)-carboxylate (12b, 230 mg, 678.01 umol, 1 eq), 2-(3,4-dihydronaphthalen-2-yl)-4,4,5,5-tetramethyl-1,3,2-dioxaborolane (12c, 694.68 mg, 2.71 mmol, 4 eq), Pd(PPh3)4 (78.35 mg, 67.80 umol, 0.1 eq), K2CO3 (374.82 mg, 2.71 mmol, 4 eq) in toluene (16 mL) was degassed and purged with N2 3 times, and then the mixture was stirred at 120° C. for 16 hours under N2 atmosphere. LCMS showed trace 12b remained and desired mass was detected. The aqueous phase was extracted with ethyl acetate (20 mL*3). The combined organic phase was washed with brine (20 mL), dried with anhydrous Na2SO4, filtered and concentrated in vacuo. The crude product was purified by column chromatography (SiO2, Petroleum ether:Ethyl acetate=20:1 to 3:1) to give 12d as a yellow oil. 1H NMR (400 MHz, CDCl3-d) δ 7.56-7.48 (m, 1H), 7.33 (d, J=7.8 Hz, 1H), 7.14-7.02 (m, 6H), 6.58 (br s, 1H), 4.06-3.96 (m, 2H), 3.54 (br t, J=5.4 Hz, 2H), 2.86-2.74 (m, 4H), 2.59 (br s, 2H), 1.36 (s, 9H).

### Tert-butyl 4-(6-(1,2,3,4-tetrahydronaphthalen-2-yl)pyridin-2-yl)piperidine-1-carboxylate (12e)

A mixture of tert-butyl 6-(3,4-dihydronaphthalen-2-yl)-5′,6′-dihydro-[2,4′-bipyridine]-1′(2′H)-carboxylate (12d, 160 mg, 411.84 umol, 1 eq), H2 (830.22 ug, 411.84 umol, 1 eq), Pd/C (5 mg, 10% purity) in MeOH (1 mL) was degassed and purged with H2 3 times, and then the mixture was stirred at 20° C. for 10 hours under H2 atmosphere. LCMS showed 12d was consumed completely and desired mass was detected. The reaction mixture was filtered. The filtrate was concentrated under reduced pressure to give 12e as light yellow oil. 1H NMR (400 MHz, CDCl3-d) δ 7.58 (t, J=7.8 Hz, 1H), 7.13 (s, 4H), 7.05 (d, J=7.8 Hz, 1H), 6.99 (d, J=7.8 Hz, 1H), 3.19-3.06 (m, 3H), 3.04-2.92 (m, 2H), 2.91-2.79 (m, 3H), 2.24-2.16 (m, 1H), 1.80-1.70 (m, 2H), 1.49 (s, 9H).

### 2-(piperidin-4-yl)-6-(1,2,3,4-tetrahydronaphthalen-2-yl)pyridine (12f)

A mixture of tert-butyl 4-(6-(1,2,3,4-tetrahydronaphthalen-2-yl)pyridin-2-yl)piperidine-1-carboxylate (12e, 97 mg, 247.11 umol, 1 eq) in HCl/EtOAc (4 M, 5 mL) was stirred at 20° C. for 0.5 hour. TLC Petroleum ether:Ethyl acetate=3:1) showed 12e was consumed completely and one new spot formed. The reaction mixture was blow-dried with N2 to give 12f (HCl salt) as a white solid. 1H NMR (400 MHz, DMSO-d6) δ 8.11 (br s, 1H), 7.45 (br s, 2H), 7.12 (t, J=4.8 Hz, 4H), 3.39 (br d, J=12.2 Hz, 1H), 3.12-2.95 (m, 4H), 2.93-2.86 (m, 2H), 2.67 (br d, J=1.8 Hz, 3H), 2.33 (br s, 1H), 2.06-1.92 (m, 3H).

### Methyl 1-((S)-oxetan-2-ylmethyl)-2-((4-(6-(1,2,3,4-tetrahydronaphthalen-2-yl)pyridin-2-yl)piperidin-1-yl)methyl)-1H-benzo[d]imidazole-6-carboxylate (12g)

To a solution of 2-(piperidin-4-yl)-6-(1,2,3,4-tetrahydronaphthalen-2-yl)pyridine (12f, 81 mg, 246.29 umol, 1 eq, HCl) in MeCN (10 mL) was added K2CO3 (170.20 mg, 1.23 mmol, 5 eq) and (S)-methyl 2-(chloromethyl)-1-(oxetan-2-ylmethyl)-1H-benzo[d]imidazole-6-carboxylate (1k, 79.85 mg, 270.92 umol, 1.1 eq). The mixture was stirred at 80° C. for 3 hours. LCMS showed 12f was consumed completely and desired mass was detected. The aqueous phase was extracted with ethyl acetate (30 mL*3). The combined organic phase was washed with brine (30 mL), dried with anhydrous Na2SO4, filtered and concentrated in vacuo. The mixture was purified by preparative TLC (Ethyl acetate) to give 12g as a white solid. 1H NMR (400 MHz, CDCl3-d) δ 8.18 (d, J=1.0 Hz, 1H), 7.97 (dd, J=1.5, 8.4 Hz, 1H), 7.75 (d, J=8.4 Hz, 1H), 7.55 (t, J=7.8 Hz, 1H), 7.16-7.09 (m, 4H), 7.01 (dd, J=7.8, 13.9 Hz, 2H), 5.24 (dq, J=3.2, 6.8 Hz, 1H), 4.82-4.67 (m, 2H), 4.65-4.57 (m, 1H), 4.41 (td, J=5.8, 9.0 Hz, 1H), 4.00-3.92 (m, 5H), 3.18-2.91 (m, 7H), 2.83-2.68 (m, 2H), 2.54-2.43 (m, 1H), 2.39-2.13 (m, 4H), 1.97-1.81 (m, 4H).

### 1-((S)-oxetan-2-ylmethyl)-2-((4-(6-(1,2,3,4-tetrahydronaphthalen-2-yl)pyridin-2-yl)piperidin-1-yl)methyl)-1H-benzo[d]imidazole-6-carboxylic acid (Compound 12)

To a solution of methyl 1-((S)-oxetan-2-ylmethyl)-2-((4-(6-(1,2,3,4-tetrahydronaphthalen-2-yl)pyridin-2-yl)piperidin-1-yl)methyl)-1H-benzo[d]imidazole-6-carboxylate (12g, 100 mg, 181.59 umol, 1 eq) in THF (15 mL) was added LiOH·H2O (22.86 mg, 544.77 umol, 3 eq) and H2O (7 mL). The mixture was stirred at 20° C. for 16 hours. LCMS showed 12g was consumed completely and desired mass was detected. 10% citric acid was added to the reaction mixture dropwise until pH=6. The aqueous phase was concentrated in vacuo. The residue was dissolved in DMSO (3 mL), the obtained solution was added to H2O (9 ml) dropwise under stirring. The mixture was stirred for 20 minutes, and filtered to collect solid. The solid was washed with H2O (3 mL*3) and concentrated in vacuo to give Compound 12 as a white solid. MS MS calculated for [M+H]+ (C33H36N4O3) requires m/z 537.3, LCMS found m/z 537.3. 1H NMR (400 MHz, MeOH-d4) δ 8.33 (s, 1H), 7.98 (d, J=8.4 Hz, 1H), 7.72-7.64 (m, 2H), 7.15 (t, J=8.4 Hz, 2H), 7.08 (s, 4H), 5.32-5.24 (m, 1H), 4.94-4.87 (m, 1H), 4.79-4.69 (m, 1H), 4.67-4.58 (m, 1H), 4.46 (td, J=5.8, 9.0 Hz, 1H), 4.16-4.08 (m, 1H), 4.03 (s, 1H), 3.21-3.02 (m, 5H), 3.02-2.88 (m, 2H), 2.87-2.75 (m, 2H), 2.59-2.38 (m, 3H), 2.15 (br d, J=12.2 Hz, 1H), 2.07-1.89 (m, 5H).

### Example 13 (General Procedure M)

### (S)-2-((4-(6-(6-cyano-8-fluoro-3,4-dihydroisoquinolin-2(1H)-yl)pyridin-2-yl)piperidin-1-yl)methyl)-1-(oxetan-2-ylmethyl)-1H-benzo[d]imidazole-6-carboxylic acid

The title compound was prepared according to Scheme 9. This General Procedure M exemplifies Scheme 9 and provides particular synthetic details as applied to the title compound.

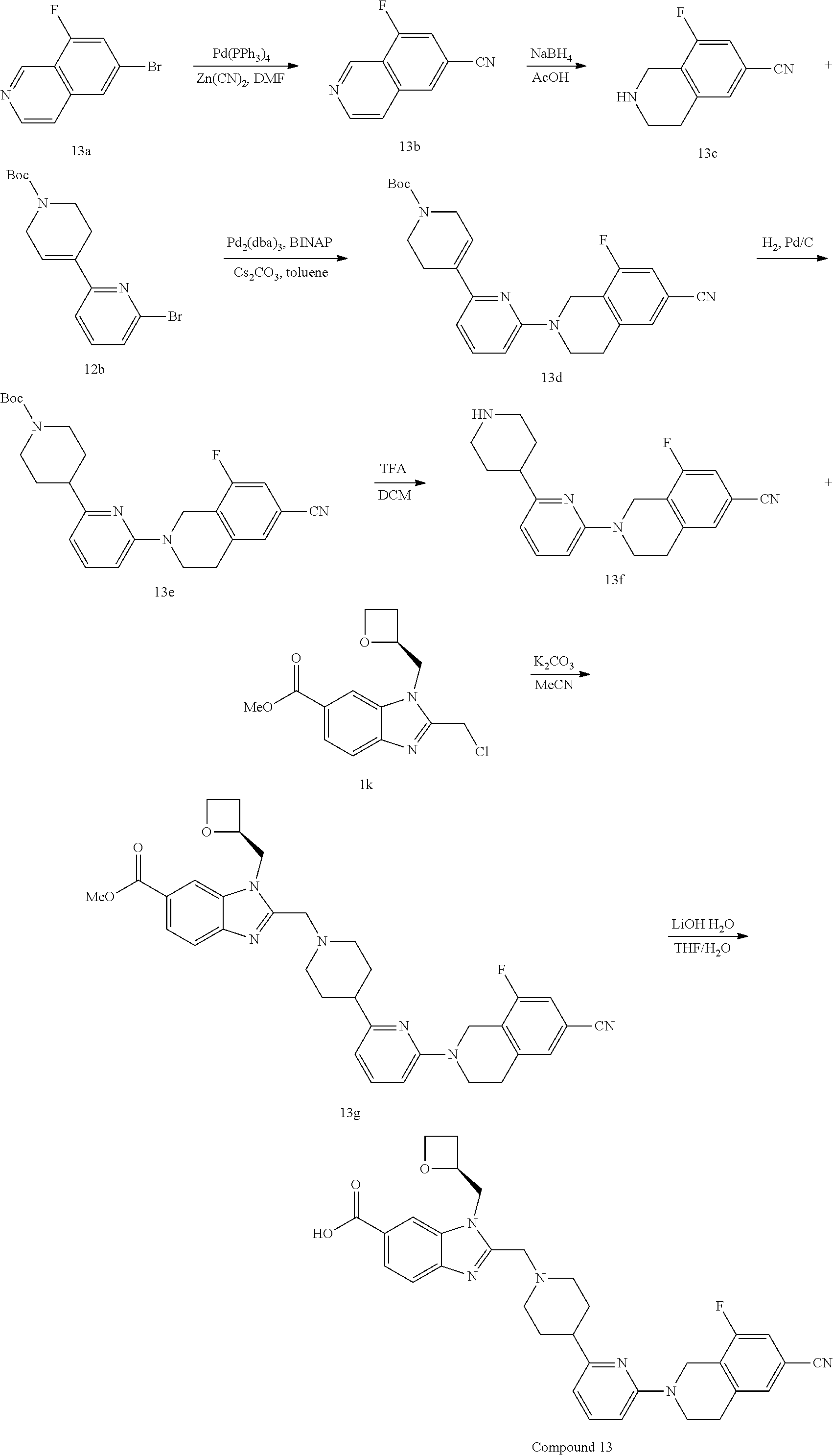

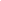

### 8-fluoroisoquinoline-6-carbonitrile (13b)

To a solution of 6-bromo-8-fluoroisoquinoline (13a, 700 mg, 3.10 mmol, 1 eq) in DMF (10 mL) was added Zn(CN)2 (545.45 mg, 4.65 mmol, 294.84 uL, 1.5 eq) and Pd(PPh3)4 (357.85 mg, 309.67 umol, 0.1 eq). The mixture was stirred at 150° C. for 1 hour under M.W. under N2. TLC (Petroleum ether:Ethyl acetate=3:1) indicated of 13a was consumed completely, and one major new spot was formed. The mixture was quenched with saturated NaHCO3 until pH>8, and extracted with ethyl acetate (80 mL*3), the combined organic phase was washed with brine (20 mL), dried with anhydrous Na2SO4, filtered and concentrated in vacuo. The residue was purified by column chromatography (SiO2, Petroleum ether/Ethyl acetate=10:1 to 3:1) to give 13b as a yellow solid.

### 8-fluoro-1,2,3,4-tetrahydroisoquinoline-6-carbonitrile (13c)

To a solution of 8-fluoroisoquinoline-6-carbonitrile (13b, 200 mg, 1.16 mmol, 1 eq) in AcOH (6 mL) was added NaBH4 (65.93 mg, 1.74 mmol, 1.5 eq). The mixture was stirred at 0-20° C. for 5 hours. TLC (Petroleum ether:Ethyl acetate=1:1) indicated of 13b was consumed completely, and one new spot was formed. The reaction mixture was quenched by addition of MeOH (10 mL) at 20° C. and addition NaHCO3 (aq) was adjust the pH>7 and then diluted with H2O (20 mL) and extracted with Ethyl acetate (20 mL*2). The combined organic layers were washed with brine (10 mL), dried over Na2SO4, filtered and concentrated under reduced pressure. The residue was purified by prep-TLC (SiO2, Ethyl acetate:Methanol=1:1) to give 13c as a white solid. 1HNMR (400 MHz, MeOH-d4) δ 7.37 (s, 1H), 7.33-7.28 (m, 1H), 4.01 (s, 2H), 3.09 (t, J=6.0 Hz, 2H), 2.91-2.84 (m, 2H).

### Tert-butyl 6-(6-cyano-8-fluoro-3,4-dihydroisoquinolin-2(1H)-yl)-5′,6′-dihydro-[2,4′-bipyridine]-1′(2′H)-carboxylate (13d)

A mixture of 8-fluoro-1,2,3,4-tetrahydroisoquinoline-6-carbonitrile (13c, 140 mg, 794.60 umol, 1 eq) and tert-butyl 6-bromo-5′,6′-dihydro-[2,4′-bipyridine]-1′(2′H)-carboxylate (12b, 323.46 mg, 953.52 umol, 1.2 eq), Pd2(dba)3 (72.76 mg, 79.46 umol, 0.1 eq), Cs2CO3 (517.79 mg, 1.59 mmol, 2 eq) and BINAP (98.95 mg, 158.92 umol, 0.2 eq) in toluene (10 mL) was degassed and purged with N2 for 3 times at 25° C., and then the mixture was stirred at 100° C. for 16 hours under N2 atmosphere. LCMS showed 13c was consumed completely, and desired mass was detected. The reaction mixture was extracted with Ethyl acetate (20 mL+10 mL) and water (10 mL). The combined organic layers were washed with brine (10 mL) and dried over Na2SO4, filtered and concentrated under reduced pressure. The residue was purified by prep-TLC (SiO2, Petroleum ether:Ethyl acetate=3:1) to give 13d as a light yellow solid 1H NMR (400 MHz, CDCl3-d) δ 7.53 (t, J=7.8 Hz, 1H), 7.30 (s, 1H), 7.22 (d, J=8.8 Hz, 1H), 6.78 (d, J=7.6 Hz, 1H), 6.70-6.63 (m, 2H), 4.77 (s, 2H), 4.18-4.12 (m, 2H), 3.94 (t, J=5.8 Hz, 2H), 3.65 (br t, J=5.6 Hz, 2H), 3.00 (t, J=5.8 Hz, 2H), 2.63 (br s, 2H), 1.50 (s, 9H).

### Tert-butyl 4-(6-(6-cyano-8-fluoro-3,4-dihydroisoquinolin-2(1H)-yl)pyridin-2-yl)piperidine-1-carboxylate (13e)

A mixture of tert-butyl 6-(6-cyano-8-fluoro-3,4-dihydroisoquinolin-2(1H)-yl)-5′,6′-dihydro-[2,4′-bipyridine]-1′(2′H)-carboxylate (13d, 90 mg, 207.13 umol, 1 eq), Pd/C (9.00 mg, 10% purity) in MeOH (3 mL) was degassed and purged with H2 3 times, and then the mixture was stirred at 25° C. for 2 hours under H2 atmosphere (15 psi). TLC (Dichloromethane:Ethyl acetate=30:1) indicated 13d was consumed completely and new spot with was detected. The reaction mixture was diluted with MeOH (20 mL) and filtered. The filtrate was concentrated under reduced pressure to give crude 13e as a light yellow solid. 1H NMR (400 MHz, CDCl3-d) δ 7.48 (dd, J=7.6, 8.4 Hz, 1H), 7.29 (s, 1H), 7.24-7.18 (m, 1H), 6.57 (d, J=8.4 Hz, 1H), 6.53 (d, J=7.2 Hz, 1H), 4.73 (s, 2H), 4.24 (br s, 2H), 3.92 (t, J=5.8 Hz, 2H), 2.98 (t, J=5.6 Hz, 2H), 2.85 (br t, J=12.2 Hz, 2H), 2.78-2.63 (m, 1H), 1.93-1.84 (m, 2H), 1.82-1.62 (m, 2H), 1.50 (s, 9H).

### 8-fluoro-2-(6-(piperidin-4-yl)pyridin-2-yl)-1,2,3,4-tetrahydroisoquinoline-6-carbonitrile (13f)

To a solution of tert-butyl 4-(6-(6-cyano-8-fluoro-3,4-dihydroisoquinolin-2(1H)-yl)pyridin-2-yl)piperidine-1-carboxylate (13e, 90 mg, 206.18 umol, 1 eq) in DCM (2.5 mL) was added TFA (0.25 mL). The mixture was stirred at 20° C. for 1 hour. TLC (Petroleum ether:Ethyl acetate=1:1) indicated 13e was consumed completely, and one new spot was formed. The mixture was adjusted to pH=8 with saturated Na2CO3 (aq), and extracted with DCM (20 mL). The organic layer was dried over Na2SO4, filtered and concentrated under reduced pressure to give crude 13f as a yellow solid. The crude used directly in next step with out any further purification.

### (S)-methyl 2-((4-(6-(6-cyano-8-fluoro-3,4-dihydroisoquinolin-2(1H)-yl)pyridin-2-yl)piperidin-1-yl)methyl)-1-(oxetan-2-ylmethyl)-1H-benzo[d]imidazole-6-carboxylate (13g)

To a solution of 8-fluoro-2-(6-(piperidin-4-yl)pyridin-2-yl)-1,2,3,4-tetrahydroisoquinoline-6-carbonitrile (13f, 90 mg, 267.53 umol, 1 eq) and (S)-methyl 2-(chloromethyl)-1-(oxetan-2-ylmethyl)-1H-benzo[d]imidazole-6-carboxylate (1k, 78.85 mg, 267.53 umol, 1 eq) in MeCN (3 mL) was added K2CO3 (36.97 mg, 267.53 umol, 1 eq). The mixture was stirred at 50° C. for 16 hours. LCMS showed 13g was consumed completely, and desired mass was detected. The reaction mixture was diluted with Ethyl acetate (10 mL) and filtered. The filtrate was concentrated under reduced pressure. The residue was purified by prep-TLC (SiO2, Petroleum ether:Ethyl acetate=0:1) to give 13g as a light yellow solid. 1H NMR (400 MHz, CDCl3-d) δ 8.20 (s, 1H), 7.97 (dd, J=1.4, 8.4 Hz, 1H), 7.75 (d, J=8.4 Hz, 1H), 7.46 (t, J=7.8 Hz, 1H), 7.28 (s, 1H), 7.20 (d, J=9.0 Hz, 1H), 6.55 (t, J=8.0 Hz, 2H), 5.31-5.19 (m, 1H), 4.82-4.62 (m, 5H), 4.44 (td, J=6.0, 9.2 Hz, 1H), 4.01-3.94 (m, 5H), 3.91 (t, J=5.8 Hz, 2H), 3.05-2.94 (m, 4H), 2.84-2.72 (m, 1H), 2.67-2.44 (m, 2H), 2.40-2.24 (m, 2H), 2.00-1.76 (m, 4H).

### (S)-2-((4-(6-(6-cyano-8-fluoro-3,4-dihydroisoquinolin-2(1H)-yl)pyridin-2-yl)piperidin-1-yl)methyl)-1-(oxetan-2-ylmethyl)-1H-benzo[d]imidazole-6-carboxylic acid (Compound 13)

To a solution of (S)-methyl 2-((4-(6-(6-cyano-8-fluoro-3,4-dihydroisoquinolin-2(1H)-yl)pyridin-2-yl)piperidin-1-yl)methyl)-1-(oxetan-2-ylmethyl)-1H-benzo[d]imidazole-6-carboxylate (13g, 170 mg, 285.87 umol, 1 eq) in THF (12 mL), H2O (5 mL) was added LiOH·H2O (17.99 mg, 428.80 umol, 1.5 eq). The mixture was stirred at 20° C. for 16 hours. LCMS showed 13g was consumed, and desired mass was detected. The mixture was adjusted to pH=6 with Citric acid (aq, 1 M). The mixture was concentrated under reduced pressure to remove THF, then diluted with water (10 mL) and extracted with Ethyl acetate (30 mL). The combined organic layers were dried over Na2SO4, filtered and concentrated under reduced pressure. The crude product was purified by Prep-HPLC (Waters Xbridge Prep OBD C18 150*40 mm*10 um; mobile phase: [water (10 mM NH4HCO3)-ACN]; B %: 30%-60%, 8 min) to give Compound 13 as a white solid. MS MS calculated for [M+H]+ (C33H33FN6O3) requires m/z 581.3, LCMS found m/z 581.3; 1H NMR (400 MHz, CDCl3-d) δ 8.18 (s, 1H), 8.04 (d, J=8.6 Hz, 1H), 7.81 (d, J=8.6 Hz, 1H), 7.47 (t, J=7.8 Hz, 1H), 7.20 (d, J=9.2 Hz, 1H), 6.56 (t, J=7.2 Hz, 2H), 5.24 (br s, 1H), 4.81-4.62 (m, 5H), 4.44 (td, J=5.8, 8.9 Hz, 1H), 4.03 (br s, 2H), 3.90 (t, J=5.6 Hz, 2H), 3.18-3.02 (m, 2H), 2.97 (br t, J=5.6 Hz, 2H), 2.81-2.71 (m, 1H), 2.64 (br s, 1H), 2.54-2.28 (m, 3H), 2.83-2.26 (m, 1H), 1.94 (br d, J=8.4 Hz, 4H).

### Example 14 (General Procedure N)

### (S)-2-((4-(6-(benzo[d]thiazol-2-ylmethoxy)pyridin-2-yl)piperidin-1-yl)methyl)-1-(oxetan-2-ylmethyl)-1H-benzo[d]imidazole-6-carboxylic acid

The title compound was prepared according to Scheme 10. This General Procedure N exemplifies Scheme 10 and provides particular synthetic details as applied to the title compound.

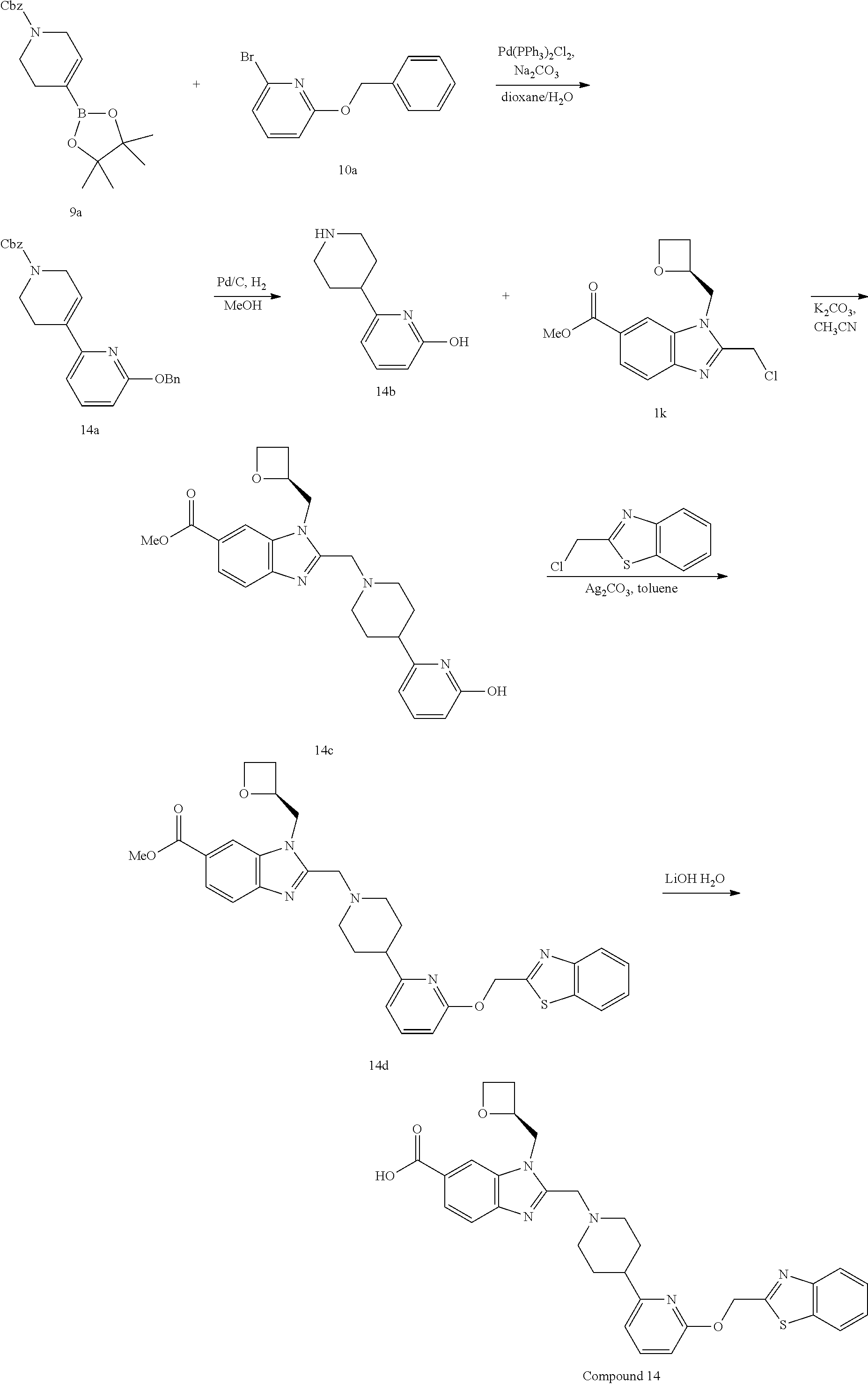

### Benzyl 6-(benzyloxy)-5′,6′-dihydro-[2,4′-bipyridine]-1′(2′H)-carboxylate (14a)

To a mixture of benzyl 4-(4,4,5,5-tetramethyl-1,3,2-dioxaborolan-2-yl)-5,6-dihydropyridine-1(2H)-carboxylate (9a, 155.94 mg, 454.34 umol, 1.2 eq) and 2-(benzyloxy)-6-bromopyridine (10a, 100 mg, 378.62 umol, 1 eq) in dioxane (2 mL) was added the mixture of sodium carbonate (120.39 mg, 1.14 mmol, 3 eq) in H2O (0.5 mL) and dichloropalladium; triphenylphosphane (13.29 mg, 18.93 umol, 0.05 eq) under N2. The mixture was stirred at 110° C. for 3 hours under N2. LCMS showed the 9a was consumed completely and one major peak with desired mass was detected. The residue was poured into water (20 mL). The aqueous phase was extracted with ethyl acetate (40 mL*2). The combined organic phase was washed with brine (30 mL*2), dried with anhydrous Na2SO4, filtered and concentrated in vacuo. The residue was purified by prep-TLC (SiO2, Petroleum ether:Ethyl acetate=3:1) to give 14a as colorless oil. 1H NMR (400 MHz, CDCl3-d) δ 7.56 (t, J=7.8 Hz, 1H), 7.46 (d, J=7.0 Hz, 2H), 7.43-7.29 (m, 8H), 6.94 (d, J=7.6 Hz, 1H), 6.76-6.65 (m, 2H), 5.44-5.39 (m, 2H), 5.19 (s, 2H), 4.23 (br d, J=2.8 Hz, 2H), 3.74 (br t, J=5.4 Hz, 2H), 2.64 (br s, 2H).

### 6-(piperidin-4-yl)pyridin-2-ol (14b)

To a solution of benzyl 6-(benzyloxy)-5′,6′-dihydro-[2,4′-bipyridine]-1′(2′H)-carboxylate (14a, 100 mg, 249.71 umol, 1 eq) in MeOH (2 mL) was added Pd/C (60 mg, 10% purity) under N2. The suspension was degassed under vacuum and purged with H2 several times. The mixture was stirred under H2 (15 psi) at 20° C. for 5 hours. LCMS showed the 14a was consumed completely and desired mass was detected. The reaction mixture was filtered and concentrated under reduced pressure to give 14b (50 mg, crude) as colorless oil. 1H NMR (400 MHz, MeOH-d4) δ 7.53 (dd, J=7.0, 8.9 Hz, 1H), 6.50-6.34 (m, 1H), 6.24 (d, J=7.0 Hz, 1H), 3.14 (br d, J=12.6 Hz, 2H), 2.77-2.58 (m, 3H), 1.88 (br d, J=10.0 Hz, 2H), 1.71-1.54 (m, 2H).

### (S)-methyl 2-((4-(6-hydroxypyridin-2-yl)piperidin-1-yl)methyl)-1-(oxetan-2-ylmethyl)-1H-benzo[d]imidazole-6-carboxylate (14c)

To a mixture of 6-(piperidin-4-yl)pyridin-2-ol (14b, 50 mg, 280.54 umol, 1 eq) and (S)-methyl 2-(chloromethyl)-1-(oxetan-2-ylmethyl)-1H-benzo[d]imidazole-6-carboxylate (1k, 74.41 mg, 252.48 umol, 0.9 eq) in CH3CN (5 mL) was added K2CO3 (193.86 mg, 1.40 mmol, 5 eq) under N2. The mixture was stirred at 50° C. for 16 hours. LCMS showed the 14b was consumed completely and desired mass was detected. The residue was poured into water (20 mL). The aqueous phase was extracted with ethyl acetate (40 mL*2). The combined organic phase was washed with brine (30 mL*2), dried with anhydrous Na2SO4, filtered and concentrated in vacuo. The residue was purified by prep-TLC (SiO2, DCM:MeOH=10:1) to give 14c as a white solid. 1H NMR (400 MHz, MeOH-d4) δ 8.34 (s, 1H), 8.06-7.87 (m, J=8.6 Hz, 1H), 7.79-7.62 (m, J=8.6 Hz, 1H), 7.52 (dd, J=7.2, 8.8 Hz, 1H), 6.36 (d, J=9.0 Hz, 1H), 6.26 (d, J=7.0 Hz, 1H), 5.31-5.05 (m, 1H), 4.80-4.62 (m, 2H), 4.45 (td, J=5.8, 9.1 Hz, 1H), 4.13-3.97 (m, 1H), 3.97-3.83 (m, 4H), 3.06 (br d, J=11.0 Hz, 1H), 2.95 (br d, J=11.4 Hz, 1H), 2.90-2.72 (m, 1H), 2.59-2.44 (m, 2H), 2.35-2.19 (m, 2H), 2.05-1.84 (m, 2H), 1.84-1.65 (m, 2H).

### (S)-methyl 2-((4-(6-(benzo[d]thiazol-2-ylmethoxy)pyridin-2-yl)piperidin-1-yl)methyl)-1-(oxetan-2-ylmethyl)-1H-benzo[d]imidazole-6-carboxylate (14d)

To a mixture of (S)-methyl 2-((4-(6-hydroxypyridin-2-yl)piperidin-1-yl)methyl)-1-(oxetan-2-ylmethyl)-1H-benzo[d]imidazole-6-carboxylate (14c, 50 mg, 114.55 umol, 1 eq) and 2-(chloromethyl)benzo[d]thiazole (23.14 mg, 126.00 umol, 1.1 eq) in toluene (2 mL) was added Ag2CO3 (63.17 mg, 229.09 umol, 10.39 uL, 2 eq) under N2. The mixture was stirred at 100° C. for 16 hours. TLC (Ethyl acetate:Methanol=10:1) indicated 14c was consumed completely and one new spot was formed. The residue was poured into water (20 mL). The aqueous phase was extracted with ethyl acetate (30 mL*2). The combined organic phase was washed with brine (30 mL*2), dried with anhydrous Na2SO4, filtered and concentrated in vacuo. The residue was purified by prep-TLC (SiO2, Ethyl acetate:Methanol=10:1) to give 14d as a yellow solid. 1H NMR (400 MHz, CDCl3-d) δ 8.20-8.16 (m, 1H), 8.02 (d, J=8.0 Hz, 1H), 7.97 (dd, J=1.6, 8.4 Hz, 1H), 7.87 (d, J=7.8 Hz, 1H), 7.75 (d, J=8.4 Hz, 1H), 7.56 (t, J=7.8 Hz, 1H), 7.48 (t, J=7.6 Hz, 1H), 7.39 (t, J=7.6 Hz, 1H), 6.80 (d, J=7.2 Hz, 1H), 6.73 (d, J=8.2 Hz, 1H), 5.84 (s, 2H), 5.22 (dq, J=3.0, 6.8 Hz, 1H), 4.80-4.57 (m, 3H), 4.39 (td, J=6.0, 9.1 Hz, 1H), 3.96 (s, 5H), 3.01-2.89 (m, 2H), 2.81-2.60 (m, 2H), 2.52-2.38 (m, 1H), 2.35-2.18 (m, 2H), 1.93-1.70 (m, 4H).

### (S)-2-((4-(6-(benzo[d]thiazol-2-ylmethoxy)pyridin-2-yl)piperidin-1-yl)methyl)-1-(oxetan-2-ylmethyl)-1H-benzo[d]imidazole-6-carboxylic acid (Compound 14)

To a mixture of (S)-methyl 2-((4-(6-(benzo[d]thiazol-2-ylmethoxy)pyridin-2-yl)piperidin-1-yl)methyl)-1-(oxetan-2-ylmethyl)-1H-benzo[d]imidazole-6-carboxylate (14d, 35 mg, 59.96 umol, 1 eq) in THF (2.1 mL) was added LiOH·H2O (2.52 mg, 59.96 umol, 1 eq) in H2O (0.9 mL) under N2. The mixture was stirred at 20° C. for 32 hours. LCMS showed 14d was remained and desired mass was detected. The mixture was quenched by addition of citric acid (10%, aq) until pH=6-7, and the reaction mixture were concentrated under reduced pressure. The residue was purified by prep-HPLC (column: Phenomenex Gemini-NX C18 75*30 mm*3 um; mobile phase: [water (10 mM NH4HCO3)-ACN]; B %: 20%-40%, 6 min) to give Compound 14 as a white solid. MS MS calculated for [M+H]+ (C31H31N504S) requires m/z 570.2, LCMS found m/z 570.2. 1H NMR (400 MHz, CDCl3-d) δ 8.15 (s, 1H), 8.08-8.00 (m, 2H), 7.85 (br d, J=7.8 Hz, 1H), 7.80 (br d, J=8.4 Hz, 1H), 7.56 (t, J=7.8 Hz, 1H), 7.47 (t, J=7.6 Hz, 1H), 7.37 (t, J=7.4 Hz, 1H), 6.79 (d, J=7.4 Hz, 1H), 6.73 (d, J=8.2 Hz, 1H), 5.83 (s, 2H), 5.19 (br d, J=4.3 Hz, 1H), 4.79-4.69 (m, 1H), 4.69-4.55 (m, 2H), 4.38 (td, J=6.0, 8.9 Hz, 1H), 4.08-3.97 (m, 2H), 3.13-3.00 (m, 2H), 2.79-2.57 (m, 2H), 2.51-2.25 (m, 3H), 1.90 (br d, J=8.6 Hz, 4H).

### Example 15 (General Procedure O)

### (S)-2-((1-(6-((4-cyano-2-fluorobenzyl)oxy)pyridin-2-yl)piperidin-4-yl)methyl)-1-(oxetan-2-ylmethyl)-1H-benzo[d]imidazole-6-carboxylic acid

The title compound was prepared according to Scheme 11. This General Procedure 0 exemplifies Scheme 11 and provides particular synthetic details as applied to the title compound.

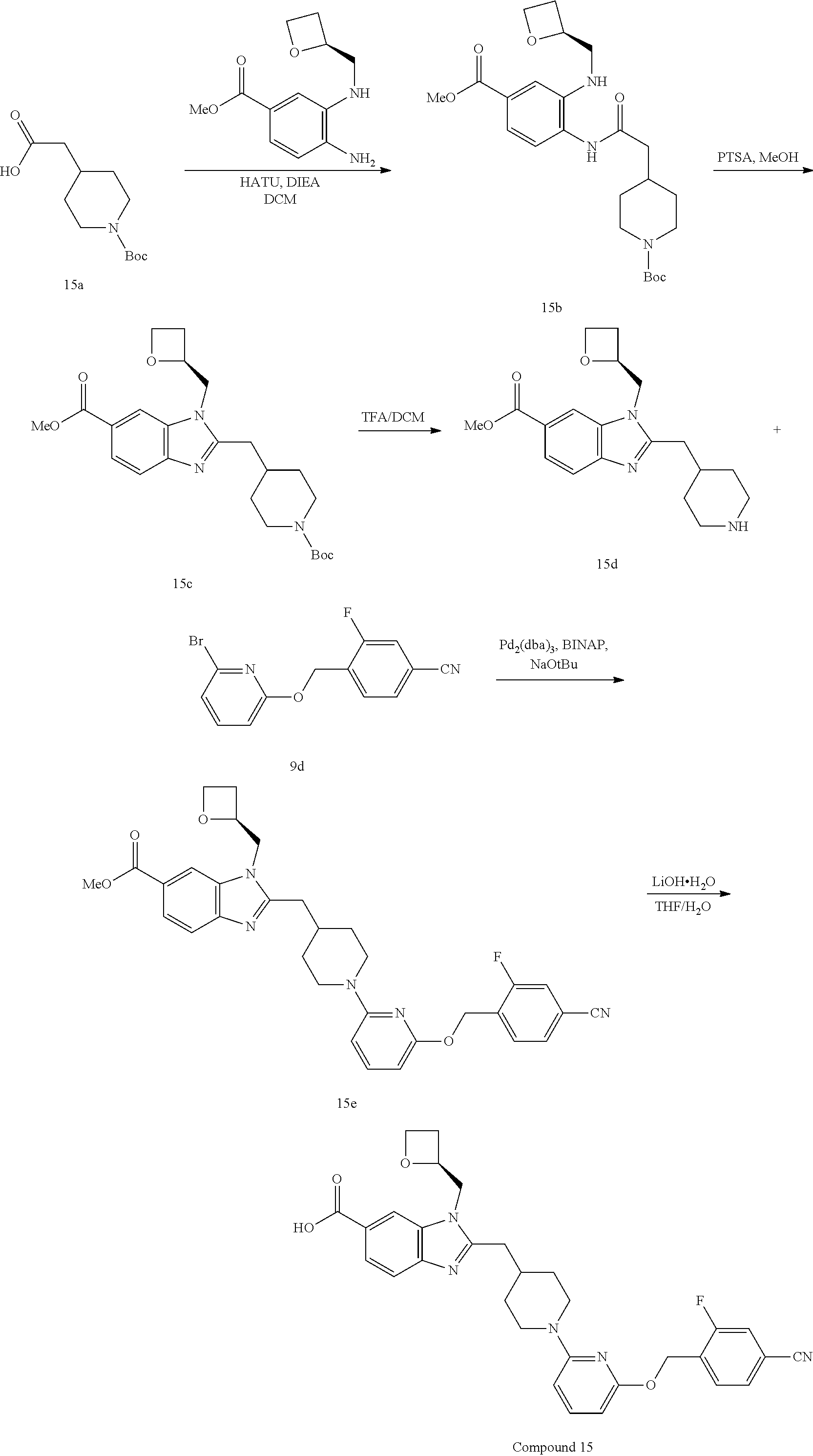

### (S)-tert-butyl 4-(2-((4-(methoxycarbonyl)-2-((oxetan-2-ylmethyl)amino)phenyl)amino)-2-oxoethyl)piperidine-1-carboxylate (15b)

HATU (579.36 mg, 1.52 mmol, 1.2 eq) and DIPEA (492.32 mg, 3.81 mmol, 663.50 uL, 3 eq) was added to the solution of 2-(1-(tert-butoxycarbonyl)piperidin-4-yl)acetic acid (15a, 386.16 mg, 1.59 mmol, 1.3 eq) in DCM (6 mL) at 20° C. Then the solution was stirred at 20° C. for 0.5 hour. Then (S)-methyl 4-amino-3-((oxetan-2-ylmethyl)amino)benzoate (300 mg, 1.27 mmol, 1 eq) was added to the solution at 20° C. Then the reaction was stirred at 20° C. for 15.5 hours. The combined organic layers were washed with brine (15 mL), dried over Na2SO4, filtered and concentrated. The residue was purified by column chromatography (SiO2, Petroleum ether/Ethyl acetate=80:1 to 20:1) to give 15b as a white solid. 1H NMR (400 MHz, CDCl3-d) δ 7.89-7.75 (m, 2H), 7.63-7.51 (m, 2H), 5.09-5.00 (m, 1H), 4.74 (br d, J=6.6 Hz, 1H), 4.65-4.57 (m, 1H), 4.17-4.04 (m, 2H), 3.91 (s, 3H), 3.46-3.25 (m, 2H), 2.75 (br s, 3H), 2.60-2.47 (m, 1H), 2.33 (br d, J=6.6 Hz, 2H), 2.14-2.02 (m, 1H), 1.83-1.72 (m, 2H), 1.57 (br s, 3H), 1.30-1.09 (m, 3H).

### (S)-methyl 2-((1-(tert-butoxycarbonyl)piperidin-4-yl)methyl)-1-(oxetan-2-ylmethyl)-1H-benzo[d]imidazole-6-carboxylate (15c)

TosOH (217.51 mg, 1.26 mmol, 1.1 eq) was added to the solution of (S)-tert-butyl 4-(2-((4-(methoxycarbonyl)-2-((oxetan-2-ylmethyl)amino)phenyl)amino)-2-oxoethyl)piperidine-1-carboxylate (15b, 530 mg, 1.15 mmol, 1 eq) in MeOH (15 mL) at 20° C. Then the solution was stirred at 80° C. for 2.5 hours. TLC (Plate 1: Dichloromethane:Methanol=20:1) and TLC (Plate 2: Petroleum ether:Ethyl acetate=0:1) showed 15b was consumed completely and detected a new main spot. The mixture was adjusted to pH=9 with aqueous NaHCO3 (10 mL). The mixture was extracted with Ethyl acetate (20 mL*3). The combined Ethyl acetate was washed with brine (15 mL), dried over Na2SO4, filtered and concentrated. The residue was purified by column chromatography (SiO2, Dichloromethane:Methanol=80:1 to 20:1) to give 15c as a colourless solid. 1H NMR (400 MHz, CDCl3-d) δ 8.08 (d, J=1.0 Hz, 1H), 7.97 (dd, J=1.6, 8.5 Hz, 1H), 7.74 (d, J=8.4 Hz, 1H), 5.22-5.13 (m, 1H), 4.62 (dt, J=6.0, 7.8 Hz, 1H), 4.47-4.31 (m, 3H), 3.95 (s, 3H), 3.50 (d, J=4.6 Hz, 3H), 2.93 (d, J=6.6 Hz, 2H), 2.81-2.69 (m, 2H), 2.80-2.68 (m, 1H), 2.47-2.37 (m, 1H), 2.30 (ddd, J=3.8, 7.6, 11.5 Hz, 1H), 1.80 (br s, 2H), 1.46 (s, 9H), 0.96 (br d, J=5.0 Hz, 1H).

### (S)-methyl 1-(oxetan-2-ylmethyl)-2-(piperidin-4-ylmethyl)-1H-benzo[d]imidazole-6-carboxylate (15d)

The solution of (S)-methyl 2-((1-(tert-butoxycarbonyl)piperidin-4-yl)methyl)-1-(oxetan-2-ylmethyl)-1H-benzo[d]imidazole-6-carboxylate (15c, 300 mg, 676.38 umol, 1 eq) in TFA (0.5 mL) and DCM (5 mL) was stirred at 20° C. for 2.5 hours. LCMS showed 15c was consumed, and desired mass was detected. The mixture was adjusted to pH=9 with saturated NaHCO3. The mixture was extracted with DCM (10 mL*3). The combined organic layer were washed with brine (15 mL), dried over Na2SO4, filtered and concentrated to give 15d as a yellow solid.

### (S)-methyl 2-((1-(6-((4-cyano-2-fluorobenzyl)oxy)pyridin-2-yl)piperidin-4-yl)methyl)-1-(oxetan-2-ylmethyl)-1H-benzo[d]imidazole-6-carboxylate (15e)

NaOtBu (179.10 mg, 1.86 mmol, 4 eq), BINAP (29.01 mg, 46.59 umol, 0.1 eq) and Pd2(dba)3 (21.33 mg, 23.30 umol, 0.05 eq) was added to the solution of (S)-methyl 1-(oxetan-2-ylmethyl)-2-(piperidin-4-ylmethyl)-1H-benzo[d]imidazole-6-carboxylate (15d, 160 mg, 465.90 umol, 1 eq) and 4-(((6-bromopyridin-2-yl)oxy)methyl)-3-fluorobenzonitrile (9d, 171.15 mg, 559.08 umol, 1.2 eq) in toluene (6 mL) at 20° C. Then the solution was stirred at 110° C. for 16 hours under N2. TLC (Ethyl acetate:Petroleum ether=3:1) showed 9d was consumed and one major new spot was formed. The mixture was filtered and the filtrate concentrated. The residue was purified by prep-TLC (Petroleum ether/Ethyl acetate=1:3) to give 15e as a colourless solid. 1H NMR (400 MHz, CDCl3-d) δ ppm 8.26 (d, J=1.0 Hz, 1H), 7.95 (dd, J=1.6, 8.4 Hz, 1H), 7.76-7.59 (m, 4H), 7.56-7.50 (m, 2H), 7.42 (t, J=8.0 Hz, 1H), 6.27 (d, J=8.2 Hz, 1H), 6.09 (d, J=7.8 Hz, 1H), 5.52-5.41 (m, 3H), 5.17 (br dd, J=2.4, 7.2 Hz, 1H), 4.70-4.54 (m, 2H), 4.54-4.47 (m, 1H), 4.43-4.34 (m, 1H), 4.23 (br d, J=13.0 Hz, 2H), 3.95-3.88 (m, 3H), 2.99-2.92 (m, 2H), 2.85-2.71 (m, 3H), 2.53-2.42 (m, 1H), 2.27 (ddd, J=3.8, 7.6, 11.2 Hz, 1H), 1.75 (br d, J=11.6 Hz, 2H), 1.35-1.26 (m, 2H).

### (S)-2-((1-(6-((4-cyano-2-fluorobenzyl)oxy)pyridin-2-yl)piperidin-4-yl)methyl)-1-(oxetan-2-ylmethyl)-1H-benzo[d]imidazole-6-carboxylic acid (Compound 15)

LiOH·H2O (11.05 mg, 263.33 umol, 1.5 eq) was added to the solution of (S)-methyl 2-((1-(6-((4-cyano-2-fluorobenzyl)oxy)pyridin-2-yl)piperidin-4-yl)methyl)-1-(oxetan-2-ylmethyl)-1H-benzo[d]imidazole-6-carboxylate (15e, 0.1 g, 175.55 umol, 1 eq) in THF (7 mL) and H2O (3 mL) at 20° C. for 48 h. LCMS detected the desired product MS and showed that most of 15e was consumed. The mixture was adjusted to pH=7 with HOAc. The mixture was extracted with Ethyl acetate (20 mL*3). The combined organic layers were washed with brine (15 mL), dried over Na2SO4, filtered and concentrated. The residue was purified by prep-HPLC (column: Waters Xbridge Prep OBD C18 150*40 mm*10 um; mobile phase: [water (10 mM NH4HCO3)-ACN]; B %: 20%-50%, 8 min) to give Compound 15 (50.39 mg, 89.55 umol, 51.01% yield, 98.74% purity) as a white solid. MS mass calculated for [M+H]+ (C31H30FN5O4) requires m/z 556.2, LCMS found m/z 556.3. 1H NMR (400 MHz, CDCl3-d) δ 8.24 (s, 1H), 7.99-7.94 (m, 1H), 7.67-7.60 (m, 2H), 7.57-7.51 (m, 2H), 7.42 (t, J=7.8 Hz, 1H), 6.28 (d, J=8.0 Hz, 1H), 6.09 (d, J=7.8 Hz, 1H), 5.44 (s, 2H), 5.18 (br d, J=7.4 Hz, 1H), 4.69-4.56 (m, 2H), 4.54-4.47 (m, 1H), 4.40 (td, J=6.2, 9.0 Hz, 1H), 4.24 (br d, J=13.0 Hz, 2H), 2.96 (br d, J=6.4 Hz, 2H), 2.79 (br t, J=11.6 Hz, 3H), 2.53-2.43 (m, 1H), 2.28 (br s, 1H), 1.75 (br d, J=12.4 Hz, 2H), 1.29 (q, J=11.8 Hz, 2H).

### Example 16 (General Procedure P)

### (S)-2-((4-(6-((5-Cyanopyrimidin-2-yl)methoxy)pyridin-2-yl)piperazin-1-yl)methyl)-1-(oxetan-2-ylmethyl)-1h-benzo[d]imidazole-6-carboxylic acid

The title compound was prepared according to Scheme 12. This General Procedure P exemplifies Scheme 12 and provides particular synthetic details as applied to the title compound.

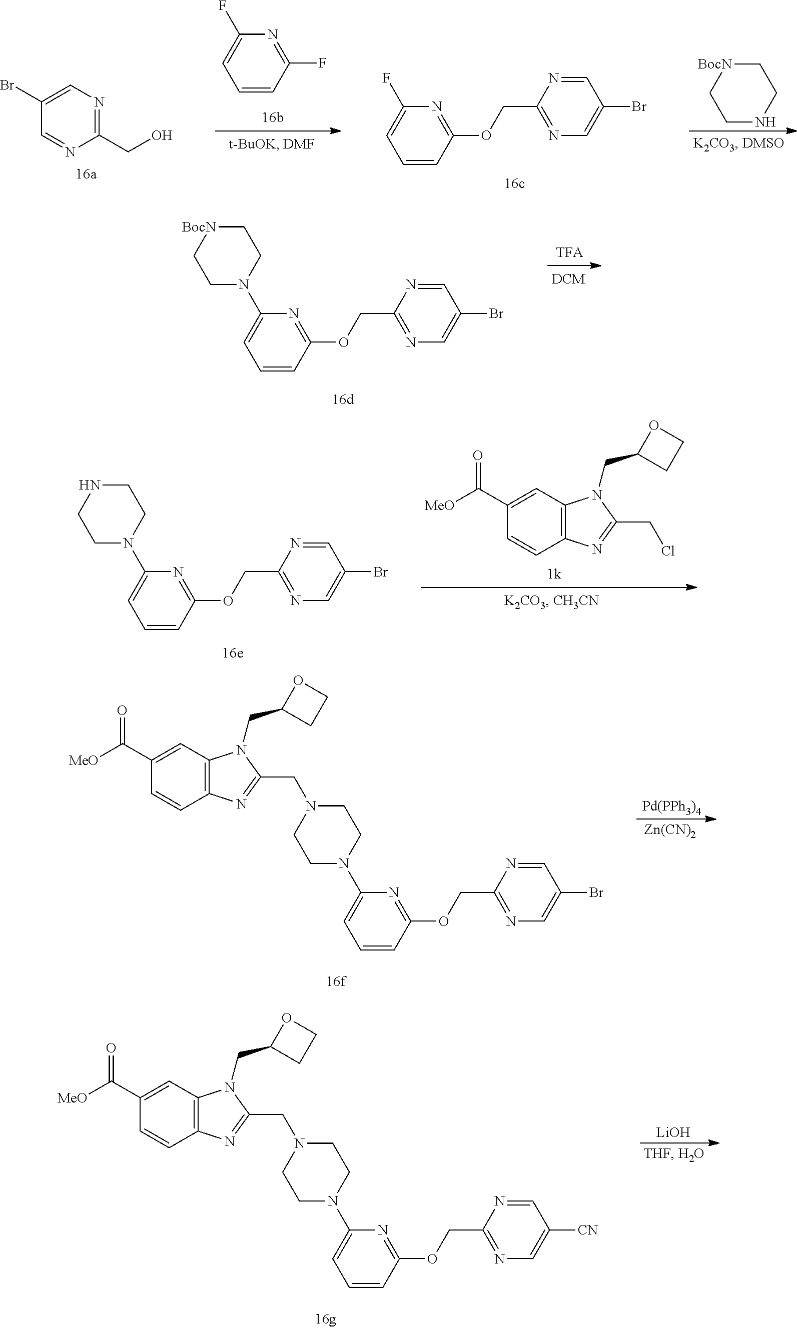

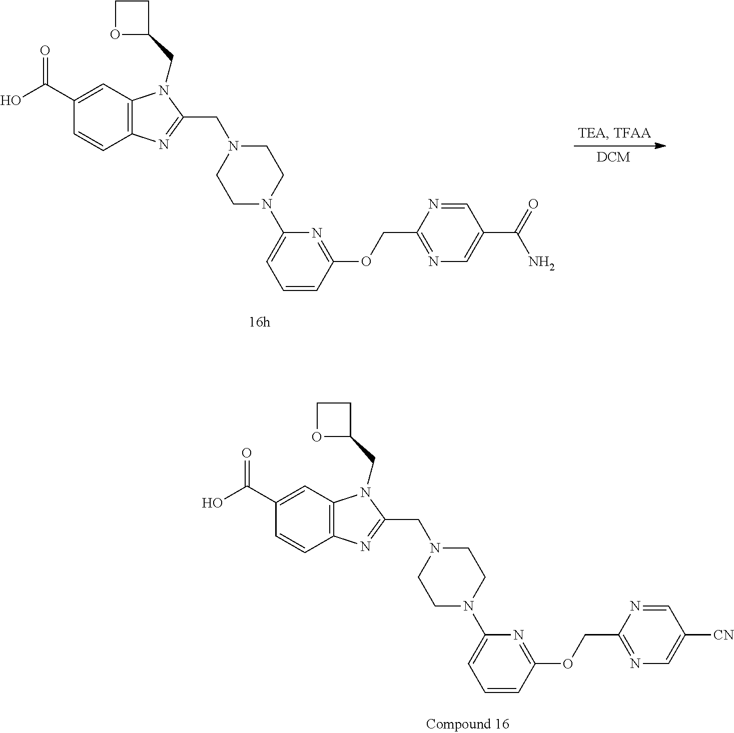

### 5-bromo-2-(((6-fluoropyridin-2-yl)oxy)methyl)pyrimidine (16c)

To a mixture of (5-bromopyrimidin-2-yl)methanol (16a, 500 mg, 2.65 mmol, 1 eq) and 2,6-difluoropyridine (16b, 365.32 mg, 3.17 mmol, 289.93 uL, 1.2 eq) in THF (5 mL) was added t-BuOK (1 M, 2.65 mL, 1 eq) at 0° C. under N2. The mixture was stirred at 20° C. for 2 hours. TLC indicated the starting material was consumed completely. The reaction mixture was poured into saturated NH4Cl aq. (10 mL), and then extracted with ethyl acetate (10 mL*3). The combined organic layers were washed with brine 10 mL, dried over anhydrous Na2SO4, filtered and concentrated under reduced pressure. The residue was purified by column chromatography (SiO2, petroleum ether:ethyl acetate=10:1 to 2:1) to give 16c as an off-white solid. 1H NMR (400 MHz, CDCl3-d) δ 8.79 (s, 2H), 7.70 (q, J=8.0 Hz, 1H), 6.81 (dd, J=1.3, 8.0 Hz, 1H), 6.50 (dd, J=2.4, 7.8 Hz, 1H), 5.54 (s, 2H).

### Tert-butyl 4-(6-((5-bromopyrimidin-2-yl)methoxy)pyridin-2-yl)piperazine-1-carboxylate (16d)

To a solution of 5-bromo-2-(((6-fluoropyridin-2-yl)oxy)methyl)pyrimidine (16c, 500 mg, 1.76 mmol, 1 eq) and tert-butyl piperazine-1-carboxylate (1.31 g, 7.04 mmol, 4 eq) in DMSO (5 mL) was added K2CO3 (729.77 mg, 5.28 mmol, 3 eq). The mixture was stirred at 130° C. for 16 hours. LCMS showed 16c was consumed, and desired mass was detected. The reaction mixture was diluted with brine (20 mL) and extracted with ethyl acetate (15 mL*3). The combined organic layers were washed with brine (10 mL), dried over anhydrous Na2SO4, filtered and concentrated under reduced pressure. The residue was purified by column chromatography (SiO2, petroleum ether:ethyl acetate=10:1 to 5:1) to give 16d as an off-white solid. MS mass calculated for [M+H]+ (C19H24BrN₅O3) requires m/z 450.2, LCMS found m/z 450.2. 1H NMR (400 MHz, CDCl3-d) δ 8.77 (s, 2H), 7.44 (t, J=8.0 Hz, 1H), 6.30 (d, J=7.8 Hz, 1H), 6.15 (d, J=8.0 Hz, 1H), 5.48 (s, 2H), 3.47-3.39 (m, 4H), 3.38-3.31 (m, 4H), 1.48 (s, 9H).

### 5-bromo-2-(((6-(piperazin-1-yl)pyridin-2-yl)oxy)methyl)pyrimidine (16e)

To a mixture of tert-butyl 4-(6-((5-bromopyrimidin-2-yl)methoxy)pyridin-2-yl)piperazine-1-carboxylate (16d, 240 mg, 532.94 umol, 1 eq) in DCM (4 mL) was added TFA (0.8 mL) under N2. The mixture was stirred at 20° C. for 2 hours. LCMS showed 16d was consumed completely and desired mass was detected. The reaction mixture was concentrated under reduced pressure to give 16e as yellow oil. MS mass calculated for [M+H]+ (C14H16BrN5O) requires m/z 350.1, LCMS found m/z 350.1; 1H NMR (400 MHz, MeOD-d4) δ 8.87 (s, 2H), 7.64-7.58 (m, 1H), 6.40-6.35 (m, 1H), 6.31 (d, J=7.8 Hz, 1H), 5.43 (s, 2H), 3.62-3.57 (m, 4H), 3.21-3.16 (m, 4H).

### (S)-methyl 2-((4-(6-((5-bromopyrimidin-2-yl)methoxy)pyridin-2-yl)piperazin-1-yl)methyl)-1-(oxetan-2-ylmethyl)-1H-benzo[d]imidazole-6-carboxylate (16f)

To a mixture of 5-bromo-2-(((6-(piperazin-1-yl)pyridin-2-yl)oxy)methyl)pyrimidine (16e, 180 mg, 513.97 umol, 1 eq) and (S)-methyl 2-(chloromethyl)-1-(oxetan-2-ylmethyl)-1H-benzo[d]imidazole-6-carboxylate (1k, 151.48 mg, 513.97 umol, 1 eq) in CH3CN (5 mL) was added K2CO3 (213.10 mg, 1.54 mmol, 3 eq) under N2. The mixture was stirred at 90° C. for 2 hours. TLC (ethyl acetatee:methanol=20:1) indicated the 16e was consumed completely and one new spot was formed. The residue was poured into water (30 mL), and extracted with ethyl acetate (50 mL*2). The combined organic layers were washed with brine (40 mL*2), dried with anhydrous Na2SO4, filtered and concentrated in vacuo. The residue was purified by prep-TLC (SiO2, ethyl acetate:methanol=20:1) to give 16f as a yellow solid. 1H NMR (400 MHz, CDCl3-d) δ 8.76 (s, 2H), 8.17 (d, J=1.0 Hz, 1H), 7.99 (dd, J=1.5, 8.6 Hz, 1H), 7.77 (d, J=8.4 Hz, 1H), 7.43 (t, J=8.0 Hz, 1H), 7.27 (s, 2H), 6.29 (d, J=7.8 Hz, 1H), 6.14 (d, J=8.0 Hz, 1H), 5.47 (s, 2H), 5.23 (tdd, J=3.2, 6.8, 9.8 Hz, 1H), 4.77-4.59 (m, 3H), 4.43-4.35 (m, 1H), 3.99-3.92 (m, 5H), 3.42-3.31 (m, 4H), 2.83-2.64 (m, 1H), 2.56 (br t, J=4.8 Hz, 4H), 2.53-2.33 (m, 1H).

### (S)-methyl 2-((4-(6-((5-cyanopyrimidin-2-yl)methoxy)pyridin-2-yl)piperazin-1-yl)methyl)-1-(oxetan-2-ylmethyl)-1H-benzo[d]imidazole-6-carboxylate (16g)

(S)-methyl 2-((4-(6-((5-bromopyrimidin-2-yl)methoxy)pyridin-2-yl)piperazin-1-yl)methyl)-1-(oxetan-2-ylmethyl)-1H-benzo[d]imidazole-6-carboxylate (16f, 140 mg, 230.08 umol, 1 eq), Zn(CN)2 (162.11 mg, 1.38 mmol, 87.63 uL, 6 eq) and Pd(PPh3)4 (26.59 mg, 23.01 umol, 0.1 eq) were taken up into a microwave tube in DMA (3 mL). The sealed tube was heated at 160° C. for 1 hour under M.W. TLC (ethyl acetatee:methanol=20:1) indicated that 16f was consumed completely and one major new spot and several minor spots were formed. The reaction mixture was filtered and the filter cake was quenched by addition NaClO(aq) (50 mL). The filtrate was poured into water (20 mL), and extracted with ethyl acetate (50 mL*2). The combined organic layers were washed with brine (40 mL*2), dried with anhydrous Na2SO4, filtered and concentrated in vacuo. The residue was purified by prep-TLC (SiO2, ethyl acetate:methanol=20:1) to give 16g as a yellow solid. 1H NMR (400 MHz, MeOD-d4) δ 9.10 (s, 2H), 8.40-8.33 (m, 1H), 7.99 (dd, J=1.5, 8.4 Hz, 1H), 7.71 (d, J=8.4 Hz, 1H), 7.46 (t, J=8.0 Hz, 1H), 6.24 (dd, J=8.0, 10.0 Hz, 2H), 5.50 (s, 2H), 5.31-5.21 (m, 1H), 4.77-4.61 (m, 2H), 4.48 (td, J=6.0, 9.2 Hz, 1H), 3.93-3.83 (m, 1H), 4.06-3.78 (m, 4H), 3.29 (br t, J=4.9 Hz, 4H), 2.83-2.64 (m, 1H), 2.58-2.43 (m, 4H), 2.53-2.33 (m, 1H).

### (S)-2-((4-(6-((5-carbamoylpyrimidin-2-yl)methoxy)pyridin-2-yl)piperazin-1-yl)methyl)-1-(oxetan-2-ylmethyl)-1H-benzo[d]imidazole-6-carboxylic acid (16h)

To a mixture of (S)-methyl 2-((4-(6-((5-cyanopyrimidin-2-yl)methoxy)pyridin-2-yl)piperazin-1-yl)methyl)-1-(oxetan-2-ylmethyl)-1H-benzo[d]imidazole-6-carboxylate (16f, 60 mg, 108.19 umol, 1 eq) in THF (0.7 mL) was added LiOH·H2O (9.08 mg, 216.38 umol, 2 eq) in H2O (0.3 mL) under N2. The mixture was stirred at 12° C. for 16 hours. LCMS showed the starting material was consumed completely and desired mass was detected. The mixture was quenched by addition of citric acid solution (10%, aq) until pH=7 and the resulting mixture were concentrated under reduced pressure. The residue was diluted in MeOH (5 mL) and filtered. The filtrate was concentrated in vacuo, and the residue was purified by prep-HPLC (column: Waters Xbridge BEH C18 100*30 mm*10 um; mobile phase: [water (10 mM NH4HCO3)-ACN]; B %: 1%-30%, 8 min) to give 16h as a white solid. MS mass calculated for [M+H]+ (C28H30N8O5) requires m/z 559.3, LCMS found m/z 559.3.1H NMR (400 MHz, MeOD-d4) δ 9.12 (s, 2H), 8.23 (s, 1H), 7.94 (s, 1H), 7.61 (d, J=8.4 Hz, 1H), 7.44 (t, J=7.8 Hz, 1H), 6.22 (dd, J=2.9, 7.9 Hz, 2H), 5.48 (s, 2H), 5.25 (br dd, J=2.6, 7.3 Hz, 1H), 4.67-4.59 (m, 3H), 4.45 (td, J=5.8, 9.1 Hz, 1H), 3.95 (s, 1H), 3.87 (s, 1H), 3.27 (br s, 4H), 2.80-2.73 (m, 1H), 2.52 (br d, J=8.6 Hz, 5H).

### (S)-2-((4-(6-((5-cyanopyrimidin-2-yl)methoxy)pyridin-2-yl)piperazin-1-yl)methyl)-1-(oxetan-2-ylmethyl)-1H-benzo[d]imidazole-6-carboxylic acid (Compound 16)

To a mixture of (S)-2-((4-(6-((5-carbamoylpyrimidin-2-yl)methoxy)pyridin-2-yl)piperazin-1-yl)methyl)-1-(oxetan-2-ylmethyl)-1H-benzo[d]imidazole-6-carboxylic acid (16h, 10 mg, 17.90 umol, 1 eq) in DCM (2 mL) was added TEA (10.87 mg, 107.41 umol, 14.95 uL, 6 eq) and TFAA (11.28 mg, 53.71 umol, 7.47 uL, 3 eq) at 0° C. under N2. The mixture was stirred at 0-20° C. for 0.5 hours. LCMS showed the 16h was consumed and desired mass was detected. The reaction mixture was concentrated under reduced pressure. The residue was purified by prep-HPLC (column: Waters Xbridge BEH C18 100*30 mm*10 um; mobile phase: [water (10 mM NH4HCO3)-ACN]; B %: 5%-35%, 8 min) to give Compound 16 as a white solid. MS mass calculated for [M+H]+ (C28H28N8O4) requires m/z 541.3, LCMS found m/z 541.3; 1H NMR (400 MHz, CDCl3-d) δ 8.96 (s, 2H), 8.26-8.20 (m, 1H), 8.05 (dd, J=1.4, 8.5 Hz, 1H), 7.82 (d, J=8.6 Hz, 1H), 7.45 (t, J=8.0 Hz, 1H), 6.31 (d, J=7.8 Hz, 1H), 6.15 (d, J=8.0 Hz, 1H), 5.57 (s, 2H), 5.24 (br s, 1H), 4.76-4.61 (m, 3H), 4.43-4.36 (m, 1H), 4.10-3.90 (m, 2H), 3.31 (br s, 4H), 2.80-2.70 (m, 1H), 2.60-2.54 (m, 4H), 2.51-2.39 (m, 1H).

### Example 17 (General Procedure Q)

### (S)-2-((4-(6-(benzyloxy)pyridin-2-yl)-2,5-dioxopiperazin-1-yl)methyl)-1-(oxetan-2-ylmethyl)-1H-benzo[d]imidazole-6-carboxylic acid

The title compound was prepared according to Scheme 13. This General Procedure Q exemplifies Scheme 13 and provides particular synthetic details as applied to the title compound.

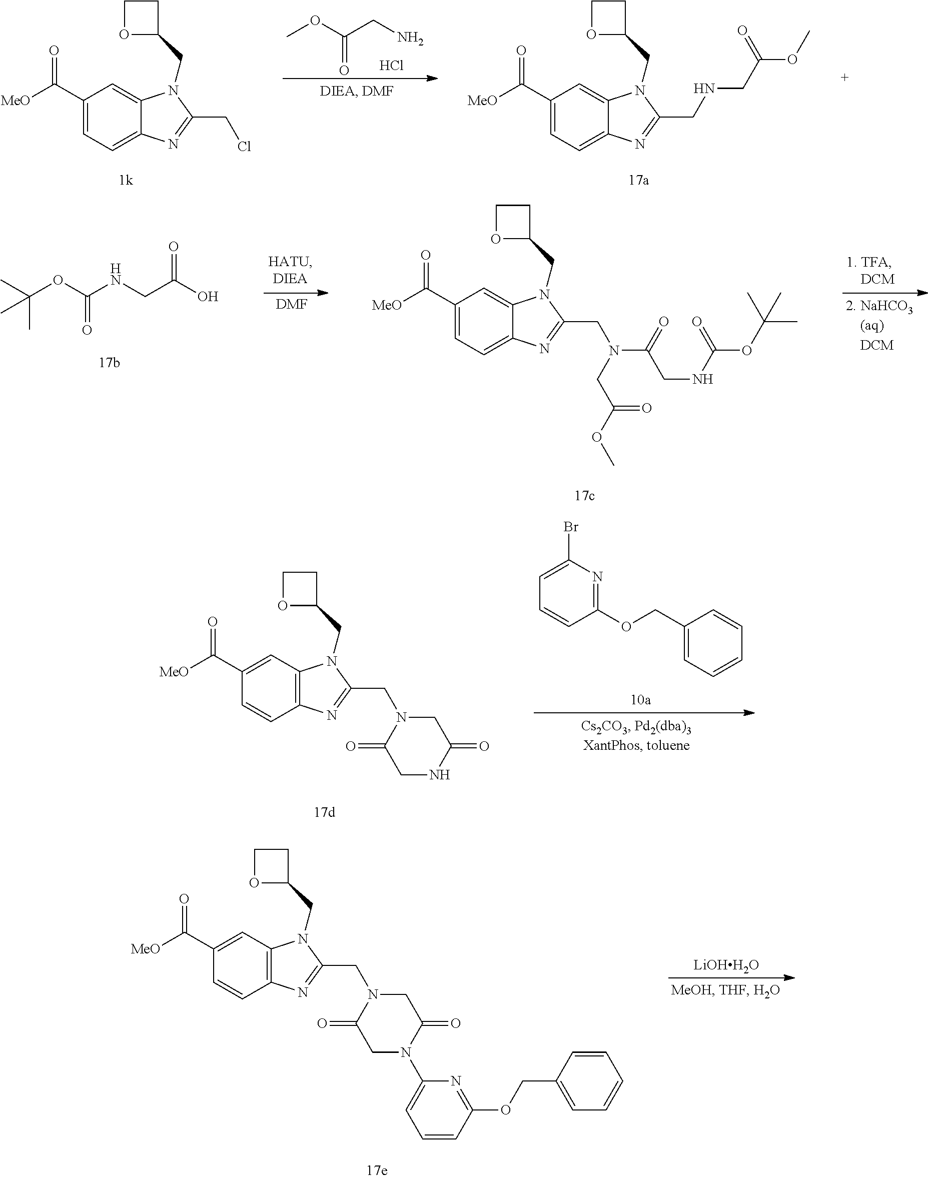

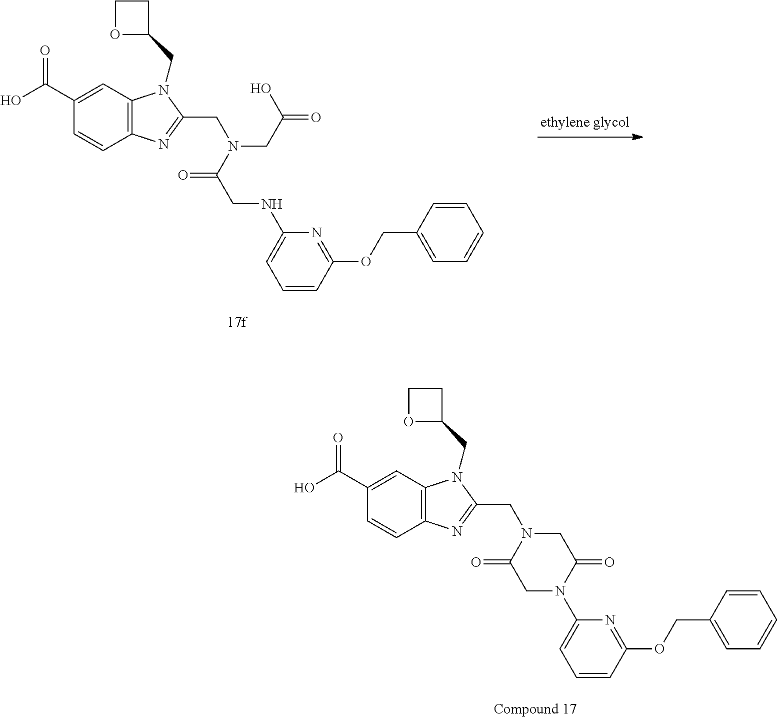 [SMILES: O=C(O)CN(Cc1nc2ccc(C(=O)O)cc2n1C[C@@H]1CCO1)C(=O)CNc1cccc(OCc2ccccc2)n1 ; O=C(O)c1ccc2nc(CN3CC(=O)N(c4cccc(OCc5ccccc5)n4)CC3=O)n(C[C@@H]3CCO3)c2c1]

### (S)-methyl 2-(((2-methoxy-2-oxoethyl)amino)methyl)-1-(oxetan-2-ylmethyl)-1H-benzo[d]imidazole-6-carboxylate (17a)

To a solution of (S)-methyl 2-(chloromethyl)-1-(oxetan-2-ylmethyl)-1H-benzo[d]imidazole-6-carboxylate (1k, 500 mg, 1.70 mmol, 1 eq) and methyl 2-aminoacetate (532.49 mg, 4.24 mmol, 2.5 eq) in DMF (6 mL) was added DIPEA (1.10 g, 8.48 mmol, 1.48 mL, 5 eq) under N2. The mixture was stirred at 40° C. for 16 hours. LCMS showed 1k was consumed completely and desired mass was detected. The mixture was extracted with ethyl acetate (30 mL*3) and H2O (10 mL). The combined organic layers were washed with brine (15 mL*3), dried with anhydrous Na2SO4, filtered and concentrated in vacuo to give 17a as an off-white solid. MS mass calculated for [M+H]+ (C17H21N3O5) requires m/z 348.4, LCMS found m/z 348.4.

### (S)-methyl 2-((2-((tert-butoxycarbonyl)amino)-N-(2-methoxy-2-oxoethyl)acetamido)methyl)-1-(oxetan-2-ylmethyl)-1H-benzo[d]imidazole-6-carboxylate (17c)

To a stirred solution of 2-((tert-butoxycarbonyl)amino)acetic acid (17b, 409.51 mg, 2.34 mmol, 1.4 eq) and DIEA (431.60 mg, 3.34 mmol, 581.67 uL, 2 eq) in DMF (6 mL) was added HATU (857.08 mg, 2.25 mmol, 1.35 eq) in one portion. The reaction mixture was stirred at 20° C. for 5 minutes. Then (S)-methyl 2-(((2-methoxy-2-oxoethyl)amino)methyl)-1-(oxetan-2-ylmethyl)-1H-benzo[d]imidazole-6-carboxylate (17a, 580 mg, 1.67 mmol, 1 eq) in DMF (1 mL) was added in the mixture. The mixture was stirred at 20° C. for 1 hour. LCMS showed 17b was consumed completely and desired mass was detected. The mixture was extracted with ethyl acetate (30 mL*2) and H2O (15 mL). The combined organic layers were washed with brine (15 mL*3), dried with anhydrous Na2SO4, filtered and concentrated in vacuo to give 17c as yellow oil. The product was used directly in next step.

### (S)-methyl 2-((2,5-dioxopiperazin-1-yl)methyl)-1-(oxetan-2-ylmethyl)-1H-benzo[d]imidazole-6-carboxylate (17d)

To a solution of (S)-methyl 2-((2-((tert-butoxycarbonyl)amino)-N-(2-methoxy-2-oxoethyl)acetamido)methyl)-1-(oxetan-2-ylmethyl)-1H-benzo[d]imidazole-6-carboxylate (17c, 680 mg, 1.35 mmol, 1 eq) in DCM (7 mL) was added TFA (1.08 g, 9.45 mmol, 0.7 mL, 7.01 eq) at 20° C. The mixture was stirred at 20° C. for 16 hours. LCMS showed 17c was consumed completely. The mixture was extracted with dichloromethane (30 mL*2) and H2O (10 mL). The combined organic layers were washed with brine (10 mL*3), dried with anhydrous Na2SO4, filtered and concentrated in vacuo. The residue was purified by Prep-TLC (dichloromethane:methanol=10:1) to give 17d as a white solid. 1H NMR (400 MHz, CDCl3-d) δ 8.10 (d, J=1.0 Hz, 1H), 8.00 (dd, J=8.50, 1.53 Hz, 1H), 7.76 (d, J=8.6 Hz, 1H), 6.23-6.17 (m, 1H), 6.20 (br s, 1H), 5.17 (qd, J=7.0, 2.4 Hz, 1H), 5.10-4.91 (m, 2H), 4.70 (dd, J=15.6, 6.8 Hz, 1H), 4.65-4.58 (m, 1H), 4.51 (dd, J=15.6, 2.45 Hz, 1H), 4.38 (dt, J=9.2 Hz, 1H), 4.41-4.34 (m, 1H), 4.30 (d, J=2.8 Hz, 1H), 4.32-4.28 (m, 1H), 4.09 (s, 2H), 3.96 (s, 3H), 3.50 (d, J=4.8 Hz, 1H), 3.52-3.47 (m, 1H), 2.81-2.71 (m, 1H), 2.49-2.38 (m, 1H).

### (S)-2-((2-((6-(benzyloxy)pyridin-2-yl)amino)-N-(carboxymethyl)acetamido)methyl)-1-(oxetan-2-ylmethyl)-1H-benzo[d]imidazole-6-carboxylic acid (17e)

A mixture of (S)-methyl 2-((2,5-dioxopiperazin-1-yl)methyl)-1-(oxetan-2-ylmethyl)-1H-benzo[d]imidazole-6-carboxylate (17d, 80 mg, 214.84 umol, 1 eq), 2-(benzyloxy)-6-bromopyridine (10a, 113.48 mg, 429.68 umol, 2 eq), Cs2CO3 (70.00 mg, 214.84 umol, 1 eq), Xantphos (7.46 mg, 12.89 umol, 0.06 eq) and Pd2(dba)3 (9.84 mg, 10.74 umol, 0.05 eq) in Tol. (3 mL) was degassed and purged with N2 3 times, and then the mixture was stirred at 100° C. for 16 hours under N2 atmosphere. LCMS showed 17d was consumed and desired mass was detected. The suspension was filtered through a pad of Celite and the pad cake was washed with ethyl acetate (5 mL*3). The filtrate was concentrated in vacuo. The residue was purified by prep-TLC (dichloromethane:methanol=10:1) to give 17e as a white solid. 1H NMR (400 MHz, CDCl3-d) δ 8.11 (s, 1H), 8.01 (d, J=8.6 Hz, 1H), 7.78 (d, J=8.6 Hz, 1H), 7. 69-7.58 (m, 2H), 7.48-7.28 (m, 5H), 6.66 (d, J=7.6 Hz, 1H), 5.34 (s, 2 H), 5.23-5.14 (m, 1H), 5.13-5.02 (m, 2H), 4.78-4.68 (m, 3H), 4.62-4.56 (m, 1H), 4.52 (dd, J=15.8, 2.21 Hz, 1H), 4.44 (d, J=5.4 Hz, 2H), 4.41-4.33 (m, 1H), 3.96 (s, 3H), 2.82-2.72 (m, 1H), 2.51-2.40 (m, 1H).

### (S)-2-((2-((6-(benzyloxy)pyridin-2-yl)amino)-N-(carboxymethyl)acetamido)methyl)-1-(oxetan-2-ylmethyl)-1H-benzo[d]imidazole-6-carboxylic acid (17f)

To a solution of (S)-methyl 2-((4-(6-(benzyloxy)pyridin-2-yl)-2,5-dioxopiperazin-1-yl)methyl)-1-(oxetan-2-ylmethyl)-1H-benzo[d]imidazole-6-carboxylate (17e, 50 mg, 90.00 umol, 1 eq) in THF (0.5 mL), methanol (0.5 mL) and H2O (0.5 mL) was added LiOH·H2O (1 M, 179.99 uL, 2 eq). The mixture was stirred at 20° C. for 16 hours. LCMS showed 17e was consumed and desired mass was detected. HCl (1 M) was added to the reaction mixture drop-wise until pH=6. The reaction mixture was concentrated under reduced to give 17f as a white solid. The crude product was used directly in next step. MS mass calculated for [M+H]+ (C29H29N5O7) requires m/z 560.1, LCMS found m/z 560.1.

### S)-2-((4-(6-(benzyloxy)pyridin-2-yl)-2,5-dioxopiperazin-1-yl)methyl)-1-(oxetan-2-ylmethyl)-1H-benzo[d]imidazole-6-carboxylic acid (Compound 17)

A mixture of (S)-2-((2-((6-(benzyloxy)pyridin-2-yl)amino)-N-(carboxymethyl)acetamido)methyl)-1-(oxetan-2-ylmethyl)-1H-benzo[d]imidazole-6-carboxylic acid (17f, 45 mg, 80.42 umol, 1 eq) in ethylene glycol (1 mL) was stirred at 145° C. for 5 hours under N2. LCMS showed the most of 17f was consumed and desired mass was detected. The mixture was purified by prep-HPLC (column: Waters Xbridge BEH C18 100*30 mm*10 um; mobile phase: [water (10 mM NH4HCO3)-ACN]; B %: 5%-40%, 8 min) to give Compound 17 as an off-white solid. 1H NMR (400 MHz, MeOD-d4) δ 8.27 (s, 1H), 8.94-7.03 (m, 1H), 7.75-7.67 (m, 2H), 7.56 (d, J=7.8 Hz, 1H), 7.47-7.42 (m, 2H), 7.38-7.32 (m, 2H), 7.30-7.24 (m, 1H), 6.70 (d, J=7.8 Hz, 1H), 5.39 (s, 1H), 5.35-5.34 (m, 1H), 5.23-5.12 (m, 2H), 5.09 (s, 1H), 4.76 (dd, J=7.2, 15.8 Hz, 1H), 4.71 (s, 2H), 4.63-4.54 (m, 2H), 4.39 (td, J=5.8, 9.2 Hz, 1H), 4.34 (d, J=2.8 Hz, 2H), 2.82-2.70 (m, 1H), 2.54-2.40 (m, 1H).

### Example 18 (General Procedure R)

### (S)-2-((4-(6-(benzyloxy)pyridin-2-yl)-4-hydroxypiperidin-1-yl)methyl)-1-(oxetan-2-ylmethyl)-1H-benzo[d]imidazole-6-carboxylic acid

The title compound was prepared according to Scheme 14. This General Procedure R exemplifies Scheme 14 and provides particular synthetic details as applied to the title compound.

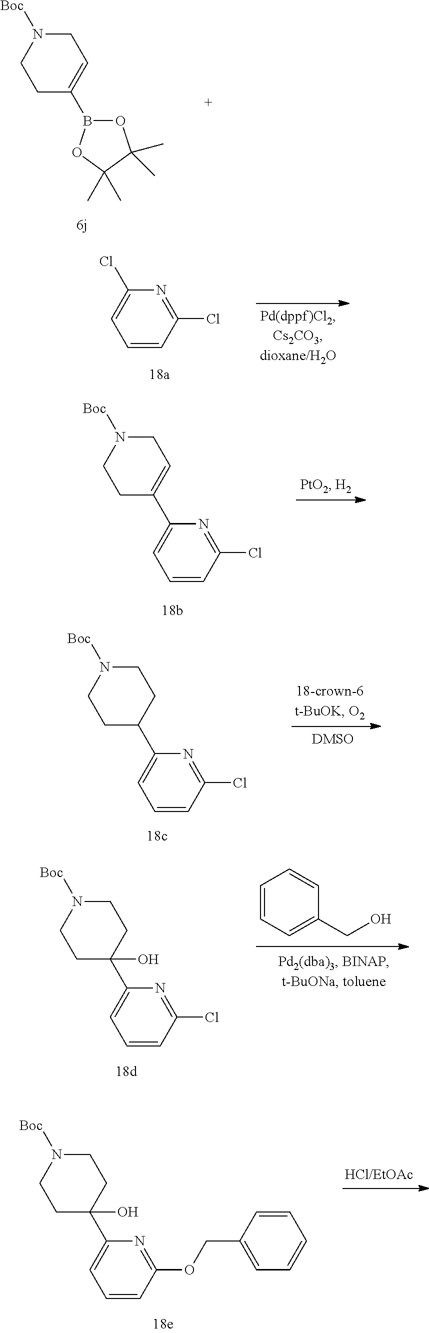

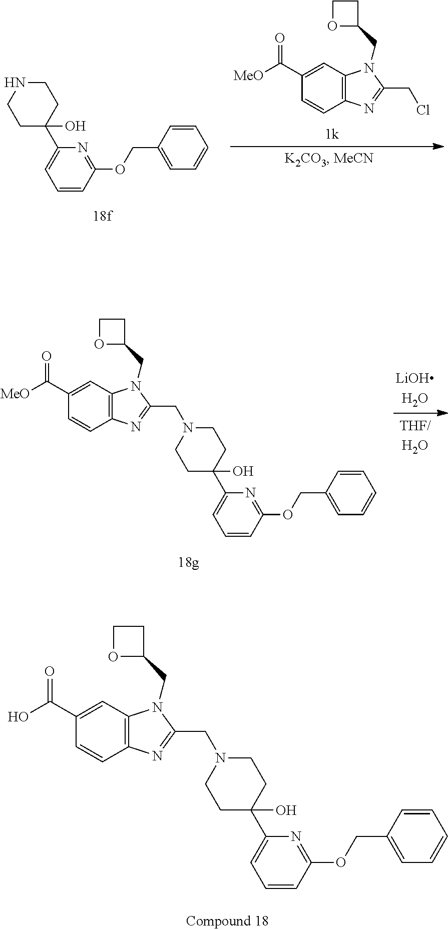 [SMILES: OC1(c2cccc(OCc3ccccc3)n2)CCNCC1 ; COC(=O)c1ccc2nc(CCl)n(C[C@@H]3CCO3)c2c1 ; COC(=O)c1ccc2nc(CN3CCC(O)(c4cccc(OCc5ccccc5)n4)CC3)n(C[C@@H]3CCO3)c2c1 ; O=C(O)c1ccc2nc(CN3CCC(O)(c4cccc(OCc5ccccc5)n4)CC3)n(C[C@@H]3CCO3)c2c1]

### Tert-butyl 6-chloro-5′,6′-dihydro-[2,4′-bipyridine]-1′(2′H)-carboxylate (18b)

Pd(dppf)Cl₂ (354.96 mg, 485.11 umol, 0.1 eq) and Cs2CO3 (3.48 g, 10.67 mmol, 2.2 eq) was added to the solution of 2,6-dichloropyridine (18a, 1.44 g, 9.70 mmol, 2 eq) and tert-butyl 4-(4,4,5,5-tetramethyl-1,3,2-dioxaborolan-2-yl)-5,6-dihydropyridine-1(2H)-carboxylate (6j, 1.5 g, 4.85 mmol, 1 eq) in dioxane (15 mL) and H2O (3 mL) at 20° C. Then the solution was stirred at 95° C. for 16 hours under N2. TLC (Plate 1: petroleum ether:ethyl acetatee=5:1) and TLC (Plate 2: petroleum ether:ethyl acetate=5:1) showed trace of 6j was remained and one new major spot was formed. The mixture was extracted with ethyl acetate (20 mL*3). The combined organic layers were washed with brine (50 mL), dried over Na2SO4, filtered and concentrated. The residue was purified by column chromatography (SiO2, petroleum ether/ethyl acetate=80:1 to 20:1) to give 18b as colorless oil. 1H NMR (400 MHz, MeOD-d4) δ 7.73 (t, J=7.8 Hz, 1H), 7.45 (d, J=7.8 Hz, 1H), 7.25 (d, J=7.8 Hz, 1H), 6.69 (br s, 1H), 4.12 (br s, 2H), 3.63 (t, J=5.6 Hz, 2H), 2.64-2.55 (m, 2H), 1.52-1.46 (m, 9H).

### Tert-butyl 4-(6-chloropyridin-2-yl)piperidine-1-carboxylate (18c)

Tert-butyl 6-chloro-5′,6′-dihydro-[2,4′-bipyridine]-1′(2′H)-carboxylate (18b, 00 mg, 1.70 mmol, 1 eq) was added to the solution of PtO2 (69.33 mg, 305.32 umol, 0.18 eq) in Ethyl acetate (6 mL) at 20° C. Then the reaction was stirred at 20° C. for 32 hours under H2 (15 Psi). TLC (petroleum ether:ethyl acetate=5:1) showed 18b was consumed and one new spot was formed. The mixture was concentrated to remove the solvent. The residue was purified by prep-TLC (petroleum ether/ethyl acetate=5:1) to give 18c as white solid. MS mass calculated for [M+H]+ (C15H21ClN2O2) requires m/z 297.0, LCMS found m/z 297.0; 1H NMR (400 MHz, MeOD-d4) δ 7.72 (t, J=7.8 Hz, 1H), 7.25 (dd, J=4.6, 7.8 Hz, 2H), 4.19 (br s, 2H), 2.88 (br d, J=3.7 Hz, 2H), 1.87 (br d, J=12.4 Hz, 2H), 1.75-1.60 (m, 2H), 1.48 (s, 9H).

### Tert-butyl 4-(6-chloropyridin-2-yl)-4-hydroxypiperidine-1-carboxylate (18d)

The solution of t-BuOK (181.48 mg, 1.62 mmol, 1.6 eq), 18-CROWN-6 (26.72 mg, 101.08 umol, 0.1 eq) and tert-butyl 4-(6-chloropyridin-2-yl)piperidine-1-carboxylate (18c, 300 mg, 1.01 mmol, 1 eq) in DMSO (10 mL) was stirred at 70° C. for 1 hour under 02. TLC (petroleum ether:ethyl acetatee=1:1) showed 18c was consumed and one new major spot was formed. The mixture was filtered and the filtrate was concentrated in vacuo. The residue was purified by column chromatography (SiO2, petroleum ether/ethyl acetatee=80:1 to 20:1) to give 18d as a white solid. 1H NMR (400 MHz, MeOD-d4) δ 7.81-7.74 (m, 1H), 7.62 (d, J=7.2 Hz, 1H), 7.28 (d, J=7.8 Hz, 1H), 3.98 (br d, J=13.2 Hz, 2H), 3.24 (br s, 2H), 2.18-2.06 (m, 2H), 1.60 (br d, J=12.6 Hz, 2H), 1.49 (s, 9H).

### Tert-butyl 4-(6-(benzyloxy)pyridin-2-yl)-4-hydroxypiperidine-1-carboxylate (18e)

T-BuONa (99.55 mg, 1.04 mmol, 3 eq), Pd2(dba)3 (15.81 mg, 17.26 umol, 0.05 eq) and BINAP (21.50 mg, 34.53 umol, 0.1 eq) were added to the solution of tert-butyl 4-(6-chloropyridin-2-yl)-4-hydroxypiperidine-1-carboxylate (18d, 108 mg, 345.28 umol, 1 eq) and phenylmethanol (186.69 mg, 1.73 mmol, 179.51 uL, 5 eq) in toluene (10 mL) at 20° C. under N2. Then the solution was stirred at 100° C. for 16 hours under N2. TLC (petroleum ether:ethyl acetatee=3:1) showed 18d was consumed and one new major spot was formed. The mixture was concentrated to remove the solvent. The residue was purified by prep-TLC (petroleum ether/ethyl acetatee=3:1) to give 18e as a yellow solid. 1H NMR (400 MHz, MeOD-d4) δ 7.70-7.61 (m, 1H), 7.46-7.16 (m, 6H), 6.69 (d, J=8.2 Hz, 1H), 5.37 (s, 2H), 3.94 (br d, J=13.2 Hz, 2H), 3.28-3.15 (m, 2H), 2.13 (dt, J=4.8, 13.2 Hz, 2H), 1.55 (br s, 2H), 1.50 (s, 9H).

### 4-(6-(benzyloxy)pyridin-2-yl)piperidin-4-ol (18f)

A solution of tert-butyl 4-(6-(benzyloxy)pyridin-2-yl)-4-hydroxypiperidine-1-carboxylate (18e, 90 mg, 234.09 umol, 1 eq) in HCl/ethyl acetate (4 M, 5 mL) was stirred at 20° C. for 0.5 hours. LCMS detected the desired mass and showed that the 18f was consumed. The mixture was concentrated under reduced pressure to give 18f as a yellow solid. MS mass calculated for [M+H]+ (C17H2ON2O2) requires m/z 285.0, LCMS found m/z 285.0. 1H NMR (400 MHz, MeOD-d4) δ 8.11 (t, J=8.2 Hz, 1H), 7.51 (d, J=7.0 Hz, 2H), 7.45-7.32 (m, 4H), 7.26-7.19 (m, 1H), 5.51 (s, 2H), 3.61 (q, J=7.0 Hz, 1H), 3.50-3.36 (m, 4H), 2.50-2.38 (m, 2H), 2.01-1.93 (m, 2H).

### (S)-methyl 2-((4-(6-(benzyloxy)pyridin-2-yl)-4-hydroxypiperidin-1-yl)methyl)-1-(oxetan-2-ylmethyl)-1H-benzo[d]imidazole-6-carboxylate (18g)

K2CO3 (193.86 mg, 1.40 mmol, 6 eq) was added to the solution of 4-(6-(benzyloxy)pyridin-2-yl)piperidin-4-ol (18f, 75 mg, 233.78 umol, 1 eq, HCl) and (S)-methyl 2-(chloromethyl)-1-(oxetan-2-ylmethyl)-1H-benzo[d]imidazole-6-carboxylate (1k, 68.90 mg, 233.78 umol, 1 eq) in CH3CN (5 mL) at 20° C. Then the solution was stirred at 50° C. for 5 hours. TLC (ethyl acetatee:methanol=8:1) showed 1k was remained and one new major spot was formed. The mixture was filtered and the filtrate was concentrated under reduced pressure. The residue was purified by prep-TLC (ethyl acetatee:methanol=8:1) to give 18g as a white solid. 1H NMR (400 MHz, MeOD-d4) δ 8.35 (s, 1H), 7.95 (dd, J=1.2, 8.4 Hz, 1H), 7.71-7.61 (m, 2H), 7.43 (d, J=7.2 Hz, 2H), 7.35-7.17 (m, 4H), 6.67 (d, J=8.2 Hz, 1H), 5.46-5.36 (m, 2H), 5.33-5.24 (m, 1H), 4.94-4.86 (m, 1H), 4.94-4.85 (m, 1H), 4.79-4.69 (m, 1H), 4.63-4.54 (m, 1H), 4.45 (td, J=5.8, 9.1 Hz, 1H), 4.10-4.00 (m, 1H), 3.93 (s, 4H), 2.86-2.75 (m, 2H), 2.74-2.47 (m, 4H), 2.40-2.25 (m, 2H), 1.68-1.53 (m, 2H).

### (S)-2-((4-(6-(benzyloxy)pyridin-2-yl)-4-hydroxypiperidin-1-yl)methyl)-1-(oxetan-2-ylmethyl)-1H-benzo[d]imidazole-6-carboxylic acid (Compound 18)

LiOH·H2O (11.75 mg, 280.12 umol, 4 eq) was added to the solution of (S)-methyl 2-((4-(6-(benzyloxy)pyridin-2-yl)-4-hydroxypiperidin-1-yl)methyl)-1-(oxetan-2-ylmethyl)-1H-benzo[d]imidazole-6-carboxylate (18g, 38 mg, 70.03 umol, 1 eq) in THF (2.1 mL) and H2O (0.9 mL) at 20° C. Then the solution was stirred at 20° C. for 20 hours. LCMS detected the desired product MS and showed that only trace 18g remained. The mixture was adjusted to pH=7 with HOAc. The resulting mixture was extracted with ethyl acetate (10 mL*3). The combined organic layers were washed with brine (15 mL), dried over Na2SO4, filtered and concentrated. The residue was purified by prep-HPLC (column: Waters Xbridge Prep OBD C18 150*40 mm*10 um; mobile phase: [water (10 mM NH4HCO3)-ACN]; B %: 15%-45%, 8 min) to give Compound 18 as a white solid. MS mass calculated for [M+H]+ (C30H32N4O5) requires m/z 529.2, LCMS found m/z 529.3; 1H NMR (400 MHz, MeOD-d4) δ 8.31 (s, 1H), 7.97 (d, J=8.4 Hz, 1H), 7.71-7.61 (m, 2H), 7.43 (d, J=7.2 Hz, 2H), 7.37-7.18 (m, 4H), 6.68 (d, J=8.2 Hz, 1H), 5.46-5.36 (m, 2H), 5.34-5.25 (m, 1H), 4.85 (br s, 1H), 4.78-4.70 (m, 1H), 4.65-4.55 (m, 1H), 4.46 (td, J=6.0, 9.1 Hz, 1H), 4.18-4.08 (m, 1H), 4.04 (s, 1H), 2.91 (br d, J=10.4 Hz, 1H), 2.85-2.70 (m, 4H), 2.60-2.48 (m, 1H), 2.37 (dq, J=4.5, 13.0 Hz, 2H), 1.64 (br t, J=11.2 Hz, 2H).

### Example 19 (General Procedure S)

### (S)-2-((4-(6-(benzyloxy)pyridin-2-yl)-3-oxopiperazin-1-yl)methyl)-1-(oxetan-2-ylmethyl)-1H-benzo[d]imidazole-6-carboxylic acid

The title compound was prepared according to Scheme 15. This General Procedure S exemplifies Scheme 15 and provides particular synthetic details as applied to the title compound.

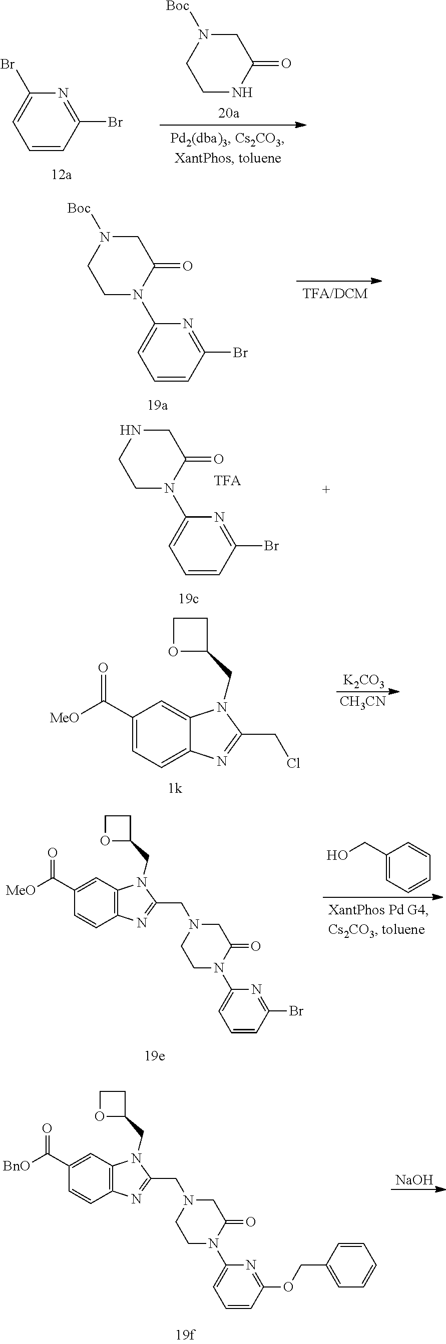

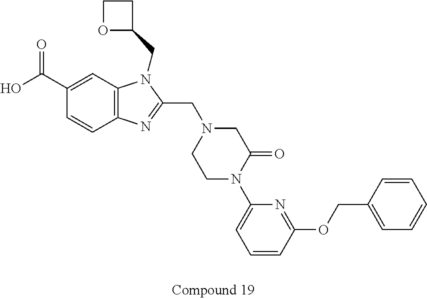 [SMILES: O=C(O)c1ccc2nc(CN3CCN(c4cccc(OCc5ccccc5)n4)C(=O)C3)n(C[C@@H]3CCO3)c2c1]

### Tert-butyl 4-(6-bromopyridin-2-yl)-3-oxopiperazine-1-carboxylatee (19b)

A mixture of 2,6-dibromopyridine (12a, 500 mg, 2.11 mmol, 1 eq), tert-butyl 3-oxopiperazine-1-carboxylate (19a, 283.16 mg, 1.41 mmol, 0.67 eq), Cs2CO3 (687.70 mg, 2.11 mmol, 1 eq), Pd2(dba)3 (96.64 mg, 105.53 umol, 0.05 eq) and Xantphos (73.28 mg, 126.64 umol, 0.06 eq) in toluene (10 mL) was degassed and purged with N2 3 times, and then the mixture was stirred at 100° C. for 16 hours under N2 atmosphere. LCMS showed desired mass was detected. The reaction mixture was concentrated under reduced pressure. The residue was purified by prep-TLC (SiO2, petroleum ether:ethyl acetate=3:1) to give 19b as a white solid. 1H NMR (400 MHz, CDCl3-d) δ 8.06 (br d, J=7.6 Hz, 1H), 7.57 (t, J=7.8 Hz, 1H), 7.30 (d, J=7.6 Hz, 1H), 4.29 (s, 2H), 4.15-4.11 (m, 2H), 3.83-3.68 (m, 2H), 1.50 (s, 9H).

### 1-(6-bromopyridin-2-yl)piperazin-2-one (19c)

To a solution of tert-butyl 4-(6-bromopyridin-2-yl)-3-oxopiperazine-1-carboxylate (19b, 130 mg, 364.95 umol, 1 eq) in DCM (10 mL) was added TFA (5.56 g, 3.61 mL). The mixture was stirred at 15° C. for 2 hours. TLC (petroleum ether:ethyl acetate=3:1) showed 19b was consumed, and one new spot was generated. The reaction mixture was concentrated under reduced pressure to give 19c as a white solid. 1H NMR (400 MHz, MeOD-d4) δ 8.01 (d, J=8.0 Hz, 1H), 7.75 (t, J=7.8 Hz, 1H), 7.48 (d, J=7.8 Hz, 1H), 4.31-4.19 (m, 2H), 4.06 (s, 2H), 3.75-3.62 (m, 2H).

### (S)-methyl 2-((4-(6-bromopyridin-2-yl)-3-oxopiperazin-1-yl)methyl)-1-(oxetan-2-ylmethyl)-1H-benzo[d]imidazole-6-carboxylate (19e)

To a solution of 1-(6-bromopyridin-2-yl)piperazin-2-one (20c, 135 mg, 364.74 umol, 1 eq, TFA) and (S)-methyl 2-(chloromethyl)-1-(oxetan-2-ylmethyl)-1H-benzo[d]imidazole-6-carboxylate (1k, 118.25 mg, 401.22 umol, 1.1 eq) in MeCN (2 mL) was added K2CO3 (252.05 mg, 1.82 mmol, 5 eq). The mixture was stirred at 60° C. for 16 hours. LCMS showed 19c was consumed, and desired mass was detected. The reaction mixture was filtered and the filtrate was concentrated under reduced pressure to give 19e as a white solid. The product was used directly in next step. 1H NMR (400 MHz, CDCl3-d) δ 8.13 (s, 1H), 8.04 (d, J=8.2 Hz, 1H), 8.00 (dd, J=1.2, 8.6 Hz, 1H), 7.78 (d, J=8.4 Hz, 1H), 7.55 (t, J=8.0 Hz, 1H), 7.29 (s, 1H), 5.26-5.15 (m, 1H), 4.75 (d, J=6.4 Hz, 1H), 4.71 (d, J=6.4 Hz, 1H), 4.68-4.58 (m, 2H), 4.36 (td, J=5.8, 9.0 Hz, 1H), 4.18-4.08 (m, 2H), 3.97 (s, 3H), 3.58-3.40 (m, 2H), 3.03-2.86 (m, 2H), 2.84-2.68 (m, 1H), 2.52-2.34 (m, 1H).

### (S)-benzyl 2-((4-(6-(benzyloxy)pyridin-2-yl)-3-oxopiperazin-1-yl)methyl)-1-(oxetan-2-ylmethyl)-1H-benzo[d]imidazole-6-carboxylate (19f)

A mixture of (S)-methyl 2-((4-(6-bromopyridin-2-yl)-3-oxopiperazin-1-yl)methyl)-1-(oxetan-2-ylmethyl)-1H-benzo[d]imidazole-6-carboxylate (19e, 50 mg, 97.21 umol, 1 eq), phenylmethanol (105.12 mg, 972.06 umol, 101.07 uL, 10 eq), (5-diphenylphosphanyl-9,9-dimethyl-xanthen-4-yl)-diphenylphosphane; (9.35 mg, 9.72 umol, 0.1 eq), Xantphos (8.44 mg, 14.58 umol, 0.15 eq), Cs2CO3 (158.36 mg, 486.03 umol, 5 eq) in toluene (2 mL) was degassed and purged with N2 3 times, and then the mixture was stirred at 100° C. for 16 hours under N2 atmosphere. LCMS showed 19e was consumed, and desired mass was detected. The reaction mixture was concentrated under reduced pressure. The residue was purified by prep-TLC (SiO2, ethyl acetate:methanol=10:1) to give 19f as a white solid. 1H NMR (400 MHz, CDCl3-d) δ 8.16 (d, J=12.2 Hz, 1H), 8.03 (dd, J=8.6, 17.8 Hz, 1H), 7.78 (d, J=8.8 Hz, 1H), 7.62 (d, J=4.2 Hz, 2H), 7.51-7.46 (m, 1H), 7.45-7.29 (m, 8H), 6.63 (t, J=4.4 Hz, 1H), 5.31 (s, 2H), 5.21 (br s, 1H), 4.79-4.69 (m, 1H), 4.68-4.58 (m, 2H), 4.37 (br d, J=7.0 Hz, 1H), 4.18-4.06 (m, 2H), 3.94 (br s, 2H), 3.59-3.40 (m, 2H), 2.91 (br d, J=9.8 Hz, 2H), 2.76 (br s, 1H), 2.45 (br s, 1H).

### (S)-2-((4-(6-(benzyloxy)pyridin-2-yl)-3-oxopiperazin-1-yl)methyl)-1-(oxetan-2-ylmethyl)-1H-benzo[d]imidazole-6-carboxylic acid (Compound 19)

To a solution of (S)-benzyl 2-((4-(6-(benzyloxy)pyridin-2-yl)-3-oxopiperazin-1-yl)methyl)-1-(oxetan-2-ylmethyl)-1H-benzo[d]imidazole-6-carboxylate (19f, 30 mg, 48.57 umol, 1 eq) in methanol (2 mL) was added NaOH (1 M, 0.6 mL, 12.35 eq) at 15° C., the mixture was stirred for 16 hours at 15° C. LCMS showed 19f was consumed, and desired mass was detected. Citric acid solution (10%, aq) was added in the mixture until pH=7, and the mixture was concentrated under reduced pressure. The residue was diluted in H2O (1 mL), and filtered. The filter cake was dried in vacuo and then purified by prep-HPLC (neutral condition; column: Waters Xbridge Prep OBD C18 150*40 mm*10 um; mobile phase: [water (10 mM NH4HCO3)-ACN]; B %: 15%-45%, 8 min) to give Compound 19 as a white solid. MS mass calculated for [M+H]+ (C29H29N5O5) requires m/z 528.2, LCMS found m/z 528.2; 1H NMR (400 MHz, DMSO-d6) δ 8.27 (s, 1H), 7.82 (d, J=10.0 Hz, 1H), 7.73 (t, J=8.0 Hz, 1H), 7.67 (d, J=8.4 Hz, 1H), 7.54 (d, J=8.0 Hz, 1H), 7.45-7.40 (m, 2H), 7.36 (t, J=7.2 Hz, 2H), 7.33-7.27 (m, 1H), 6.69 (d, J=8.0 Hz, 1H), 5.31 (s, 2H), 5.06 (br d, J=6.8 Hz, 1H), 4.84-4.74 (m, 1H), 4.70-4.60 (m, 1H), 4.51-4.41 (m, 1H), 4.35 (td, J=6.0, 9.1 Hz, 1H), 4.11-4.03 (m, 1H), 3.94 (d, J=13.8 Hz, 1H), 3.85 (br s, 2H), 3.49-3.43 (m, 2H), 3.39 (br s, 1H), 2.91 (br t, J=5.2 Hz, 2H), 2.74-2.60 (m, 1H), 2.45-2.31 (m, 1H).

### Example 20 (General Procedure T)

### 2-(((R)-3-(6-((4-cyano-2-fluorobenzyl)oxy)pyridin-2-yl)-3-methylpyrrolidin-1-yl)methyl)-1-((S)-oxetan-2-ylmethyl)-1H-benzo[d]imidazole-6-carboxylic acid

### and

### 2-(((S)-3-(6-((4-cyano-2-fluorobenzyl)oxy)pyridin-2-yl)-3-methylpyrrolidin-1-yl)methyl)-1-((S)-oxetan-2-ylmethyl)-1H-benzo[d]imidazole-6-carboxylic acid

The title compound was prepared according to Scheme 16. This General Procedure T exemplifies Scheme 16 and provides particular synthetic details as applied to the title compound.

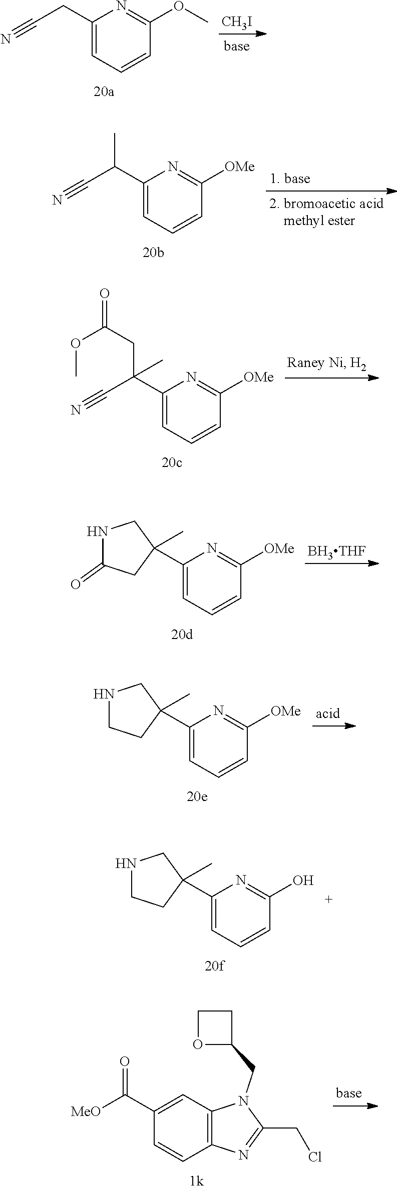

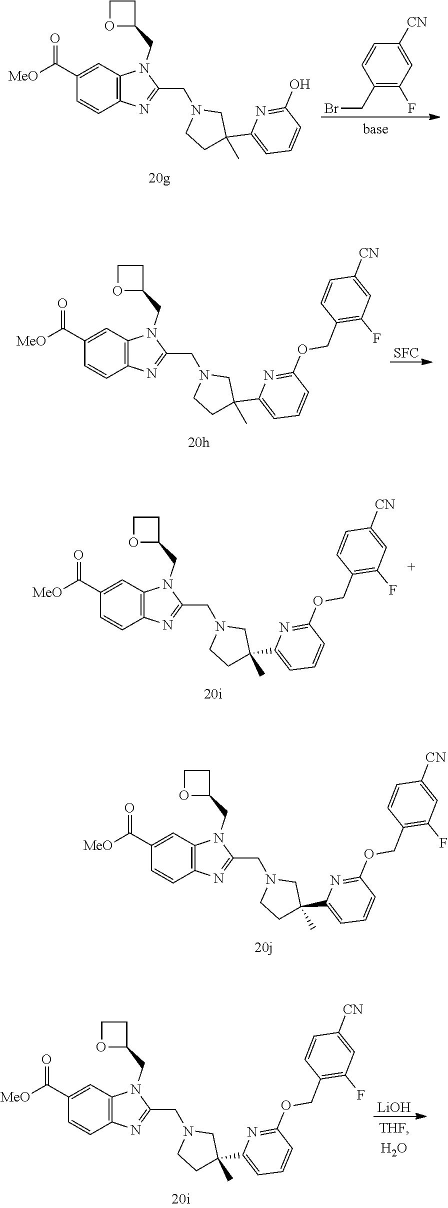

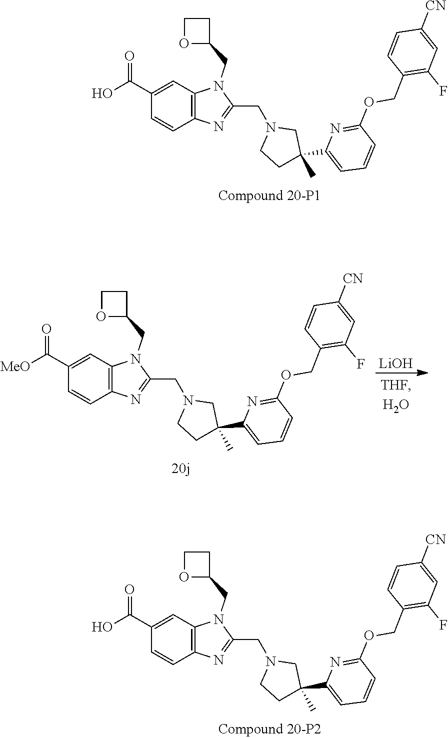

### 2-(6-methoxypyridin-2-yl)propanenitrile (20b)

t-BuOK (454.42 mg, 4.05 mmol, 1 eq) was added to the solution of 2-(6-methoxypyridin-2-yl)acetonitrile (20a, 600 mg, 4.05 mmol, 1 eq) in THF (12 mL) portion-wise at 0° C. The solution was stirred at 20° C. for 0.5 hours. Then CH3I (574.80 mg, 4.05 mmol, 252.10 uL, 1 eq) in THF (1.2 mL) was added to the reaction at 0° C. and the resulting mixture was stirred at 20° C. for 1 hour. LCMS detected the desired product mass and showed that the 20a was consumed. The mixture was concentrated under reduced pressure to give 20b as brown solid. MS mass calculated for [M+H]+ (C9H10N20) requires m/z 163.1, LCMS found m/z 163.1.

### Methyl 3-cyano-3-(6-methoxypyridin-2-yl)butanoate (20c)

NaH (221.96 mg, 110.98 mmol, 60% purity, 30.00 eq) was added to the mixture of 2-(6-methoxypyridin-2-yl)propanenitrile (20b, 600 mg, 3.70 mmol, 1 eq) in THF (12 mL) at 0° C. Then the solution was stirred at 90° C. for 0.5 hours. Then methyl 2-bromoacetate (679.10 mg, 4.44 mmol, 419.20 uL, 1.2 eq) was added to the reaction mixture at 20° C. and the mixture was stirred at 90° C. for 1 hour. TLC (petroleum ether:ethyl acetatee=5:1) showed 20b was consumed and one new spot was formed. The mixture was quenched with saturated aqueous NH4Cl (20 mL) and extracted with ethyl acetate (20 mL*3). The combined orhganic layers were washed with brine (20 mL), dried over Na2SO4, filtered and concentrated in vacuo. The residue was purified by column chromatography (SiO2, petroleum ether/ethyl acetatee=80:1 to 20:1) to give 20c as yellow liquid. 1H NMR (400 MHz, CDCl3-d) δ 7.62 (dd, J=7.4, 8.4 Hz, 1H), 7.27 (t, J=3.8 Hz, 1H), 6.68 (d, J=8.4 Hz, 1H), 3.92-3.87 (m, 3H), 3.66 (s, 3H), 3.29 (d, J=16.4 Hz, 1H), 3.01 (d, J=16.4 Hz, 1H), 1.78 (s, 3H).

### 4-(6-methoxypyridin-2-yl)-4-methylpyrrolidin-2-one (20d)

Ni (147.08 mg, 2.51 mmol, 1 eq) was added to the solution of methyl 3-cyano-3-(6-methoxypyridin-2-yl)butanoate (20c, 587 mg, 2.51 mmol, 1 eq) in EtOH (35 mL) at 20° C. Then the solution was stirred at 60° C. for 5.5 hours under H2 (50 Psi). TLC (petroleum ether:ethyl acetatee=0:1) showed 20c was consumed and one new major spot was formed. LCMS detected the desired product MS and showed that 20c was consumed. The mixture was concentrated to remove the solvent. The residue was purified by column chromatography (SiO2, petroleum ether:ethyl acetate=80:1 to 20:1) to give 20d as a white solid. MS mass calculated for [M+H]+ (C11H14N2O2) requires m/z 207.1, LCMS found m/z 207.1; 1H NMR (400 MHz, MeOD-d4) δ 7.62 (dd, J=7.4, 8.3 Hz, 1H), 6.97-6.89 (m, 1H), 6.67-6.59 (m, 1H), 3.94-3.87 (m, 3H), 3.79 (d, J=10.0 Hz, 1H), 3.41 (d, J=10.0 Hz, 1H), 3.00 (d, J=16.6 Hz, 1H), 2.36 (d, J=16.4 Hz, 1H), 1.52 (s, 3H).

### 2-methoxy-6-(3-methylpyrrolidin-3-yl)pyridine (20e)

BH3·THF (1 M, 12.77 mL, 6 eq) was added to the solution of 4-(6-methoxypyridin-2-yl)-4-methylpyrrolidin-2-one (20d, 439 mg, 2.13 mmol, 1 eq) in THF (20 mL) at 0° C. Then the solution was stirred at 80° C. for 16 hours. LCMS detected the desired product mass and showed that the reaction 20d was consumed. HCl (1 M, 2 mL) was added to the solution at 20° C. and the reaction was refluxed for 2 hours. The reaction solution was concentrated under reduced pressure to give 20e as a white solid. MS mass calculated for [M+H]+ (C11H16N2O) requires m/z 193.1, LCMS found m/z 193.1; 1H NMR (400 MHz, MeOD-d4) δ 8.28-8.21 (m, 1H), 7.88 (dd, J=7.6, 8.4 Hz, 1H), 7.33-7.26 (m, 1H), 7.18 (d, J=7.6 Hz, 1H), 6.91 (d, J=8.4 Hz, 1H), 4.02 (s, 3H), 3.92 (d, J=11.8 Hz, 1H), 3.60-3.40 (m, 3H), 3.40-3.33 (m, 1H), 2.63-2.51 (m, 1H), 2.24 (td, J=7.4, 13.3 Hz, 1H), 1.55 (s, 3H).

### 6-(3-methylpyrrolidin-3-yl)pyridin-2-ol (20f)

The solution of 2-methoxy-6-(3-methylpyrrolidin-3-yl)pyridine (20e, 150 mg, 780.21 umol, 1 eq) in HBr (4 mL) was stirred at 140° C. for 12 hours. LCMS detected the desired product MS and showed that the 20e was consumed. The mixture was filtered and the filtrate concentrated under reduced pressure to give 20f as a brown solid. MS mass calculated for [M+H]+ (C10H₁₄N2O) requires m/z 179.1, LCMS found m/z 179.1; 1H NMR (400 MHz, MeOD-d4) δ 8.24 (dd, J=7.8, 8.8 Hz, 1H), 7.28 (d, J=7.4 Hz, 1H), 7.15 (d, J=8.2 Hz, 1H), 3.82-3.73 (m, 1H), 3.64-3.47 (m, 3H), 3.35 (s, 1H), 2.64-2.53 (m, 1H), 2.42 (s, 1H), 1.59 (s, 2H).

### Methyl 2-((3-(6-hydroxypyridin-2-yl)-3-methylpyrrolidin-1-yl)methyl)-1-((S)-oxetan-2-ylmethyl)-1H-benzo[d]imidazole-6-carboxylate (20g)

K2CO3 (504.03 mg, 3.65 mmol, 5 eq) was added to the solution of 6-(3-methylpyrrolidin-3-yl)pyridin-2-ol (20f, 130 mg, 729.39 umol, 1 eq) and (S)-methyl 2-(chloromethyl)-1-(oxetan-2-ylmethyl)-1H-benzo[d]imidazole-6-carboxylate (1k, 161.23 mg, 547.04 umol, 0.75 eq) in CH3CN (10 mL) at 20° C. Then the solution was stirred at 50° C. for 2 hours. TLC (ethyl acetatee:methanol=1:1) showed 20f was consumed and one new major spot was formed. The mixture was filtered and the filtrate was concentrated under reduced pressure. The residue was purified by column chromatography (SiO2, petroleum ether:ethyl acetatee=80:1 to 20:1) to give 20g as a white solid. 1H NMR (400 MHz, MeOD-d4) δ 8.32 (s, 1H), 7.96 (dd, J=0.9, 8.5 Hz, 1H), 7.70 (d, J=8.6 Hz, 1H), 7.50 (ddd, J=4.6, 7.1, 9.0 Hz, 1H), 6.41-6.34 (m, 1H), 6.28 (s, 1H), 5.24-5.16 (m, 1H), 4.93-4.86 (m, 1H), 4.75-4.67 (m, 1H), 4.65-4.57 (m, 1H), 4.42 (dd, J=5.6, 9.0 Hz, 1H), 4.24 (dd, J=4.6, 13.6 Hz, 1H), 4.09-4.01 (m, 1H), 3.93 (s, 3H), 3.18-3.10 (m, 1H), 3.07-3.00 (m, 1H), 2.85-2.65 (m, 2H), 2.54 (s, 2H), 2.16 (dtd, J=4.2, 8.4, 12.8 Hz, 1H), 2.01 (s, 1H), 1.46 (d, J=3.2 Hz, 3H).

### Methyl 2-((3-(6-((4-cyano-2-fluorobenzyl)oxy)pyridin-2-yl)-3-methylpyrrolidin-1-yl)methyl)-1-((S)-oxetan-2-ylmethyl)-1H-benzo[d]imidazole-6-carboxylate (20h)

Ag2CO3 (240.05 mg, 870.56 umol, 39.48 uL, 2 eq) was added to the solution of methyl 2-((3-(6-hydroxypyridin-2-yl)-3-methylpyrrolidin-1-yl)methyl)-1-((S)-oxetan-2-ylmethyl)-1H-benzo[d]imidazole-6-carboxylate (20g, 190 mg, 435.28 umol, 1 eq) and 4-(bromomethyl)-3-fluoro-benzonitrile (93.16 mg, 435.28 umol, 1 eq) in toluene (10 mL) at 20° C. Then the solution was stirred at 100° C. for 4 hours. LCMS detected the desired product mass and showed that the 20g was consumed. The mixture was filtered and the filtrate was concentrated under reduced pressure. The residue was purified by column chromatography (SiO2, petroleum ether:ethyl acetate=80:1 to 20:1) to give 20h as a yellow solid. MS mass calculated for [M+H]+ (C32H32FN5O4) requires m/z 570.2, LCMS found m/z 570.2; 1H NMR (400 MHz, MeOD-d4) δ 8.29 (s, 1H), 7.96-7.89 (m, 1H), 7.70-7.39 (m, 5H), 6.97 (dd, J=1. 2, 7.4 Hz, 1H), 6.69 (d, J=8.2 Hz, 1H), 5.45-5.26 (m, 2H), 5.15-4.99 (m, 1H), 4.81-4.71 (m, 1H), 4.62-4.50 (m, 2H), 4.46-4.34 (m, 1H), 4.20-4.06 (m, 1H), 4.03-3.87 (m, 4H), 3.31 (s, 7H), 3.12 (dd, J=6.8, 8.8 Hz, 1H), 2.88-2.58 (m, 4H), 2.47-2.33 (m, 2H), 1.94-1.81 (m, 1H), 1.37 (s, 3H).

Methyl 2-(((R)-3-(6-((4-cyano-2-fluorobenzyl)oxy)pyridin-2-yl)-3-methylpyrrolidin-1-yl)methyl)-1-((S)-oxetan-2-ylmethyl)-1H-benzo[d]imidazole-6-carboxylate (20i) and methyl 2-(((S)-3-(6-((4-cyano-2-fluorobenzyl)oxy)pyridin-2-yl)-3-methylpyrrolidin-1-yl)methyl)-1-((S)-oxetan-2-ylmethyl)-1H-benzo[d]imidazole-6-carboxylate (20j). Methyl 2-((3-(6-((4-cyano-2-fluorobenzyl)oxy)pyridin-2-yl)-3-methylpyrrolidin-1-yl)methyl)-1-((S)-oxetan-2-ylmethyl)-1H-benzo[d]imidazole-6-carboxylate (20h) was separated with Chiral SFC (DAICEL CHIRALPAK AD (250 mm*30 mm, 10 um); mobile phase: [0.1% NH3H2O IPA]; B %: 45%-45%, min) to give 20i as a white solid. 1H NMR (400 MHz, MeOD-d4) δ 8.29 (d, J=1.0 Hz, 1H), 7.92 (dd, J=1.4, 8.5 Hz, 1H), 7.70-7.59 (m, 2H), 7.57-7.38 (m, 3H), 6.97 (d, J=7.2 Hz, 1H), 6.69 (d, J=7.8 Hz, 1H), 5.33-5.33 (m, 1H), 5.40-5.24 (m, 1H), 5.03 (dq, J=2.4, 7.4 Hz, 1H), 4.76 (dd, J=7.6, 15.1 Hz, 1H), 4.60-4.49 (m, 2H), 4.47-4.38 (m, 1H), 4.17 (d, J=13.6 Hz, 1H), 3.99-3.85 (m, 4H), 3.12 (d, J=8.8 Hz, 1H), 2.86-2.73 (m, 2H), 2.68-2.56 (m, 2H), 2.48-2.35 (m, 2H), 1.94-1.82 (m, 1H), 1.37 (s, 3H). 20j was obtained as a white solid. 1H NMR (400 MHz, MeOD-d4) δ 8.29 (d, J=1.0 Hz, 1H), 7.93 (dd, J=1.4, 8.5 Hz, 1H), 7.71-7.54 (m, 3H), 7.52-7.39 (m, 2H), 6.97 (d, J=7.4 Hz, 1H), 6.69 (d, J=8.0 Hz, 1H), 5.48-5.34 (m, 2H), 5.11 (dq, J=2.6, 7.2 Hz, 1H), 4.75 (dd, J=7.2, 15.3 Hz, 1H), 4.64-4.51 (m, 2H), 4.38 (td, J=6.0, 9.2 Hz, 1H), 4.13-3.89 (m, 5H), 3.10 (d, J=9.2 Hz, 1H), 2.84 (br d, J=5.6 Hz, 1H), 2.72-2.59 (m, 3H), 2.46-2.32 (m, 2H), 1.86 (ddd, J=6.2, 8.4, 12.7 Hz, 1H), 1.37 (s, 3H).

### 2-(((R)-3-(6-((4-cyano-2-fluorobenzyl)oxy)pyridin-2-yl)-3-methylpyrrolidin-1-yl)methyl)-1-((S)-oxetan-2-ylmethyl)-1H-benzo[d]imidazole-6-carboxylic acid (Compound 20-P1)

LiOH·H2O (6.40 mg, 152.56 umol, 1.1 eq) was added to the solution of methyl 2-(((R)-3-(6-((4-cyano-2-fluorobenzyl)oxy)pyridin-2-yl)-3-methylpyrrolidin-1-yl)methyl)-1-((S)-oxetan-2-ylmethyl)-1H-benzo[d]imidazole-6-carboxylate (20i, 79 mg, 138.69 umol, 1 eq) in THF (5.6 mL) and H2O (2.4 mL) at 20° C. Then the solution was stirred at 20° C. for 24 hours. LCMS detected the desired product mass and showed that the 20i still remained. LiOH·H2O (6.40 mg, 152.56 umol, 1.1 eq) was added in the mixture at 20° C. Then the solution was stirred at 20° C. for another 24 hours. LCMS detected the desired product MS and showed that the 20i was consumed. The mixture was adjusted to pH=7 with HOAc. The mixture was extracted with ethyl acetate (10 mL*3). The combined organic layers were washed with brine (15 mL), dried over Na2SO4, filtered and concentrated. The residue was purified by prep-HPLC (Neutral condition, Phenomenex Gemini-NX C18 75*30 mm*3 um; mobile phase: [water (10 mM NH4HCO3)-ACN]; B %: 25%-55%, 8 min) to give Compound 20-P1 as a white solid. MS mass calculated for [M+H]+ (C31H30FN5O4) requires m/z 556.3, LCMS found m/z 556.3; 1H NMR (400 MHz, MeOD-d4) δ 8.26 (s, 1H), 7.93 (dd, J=1.2, 8.4 Hz, 1H), 7.72-7.39 (m, 5H), 6.99 (d, J=7.4 Hz, 1H), 6.71 (d, J=8.1 Hz, 1H), 5.48-5.32 (m, 2H), 5.07 (dt, J=5.2, 7.2 Hz, 1H), 4.98-4.92 (m, 1H), 4.75 (br dd, J=7.4, 15.2 Hz, 1H), 4.61-4.50 (m, 2H), 4.40 (td, J=5.8, 9.0 Hz, 1H), 4.30 (d, J=13.8 Hz, 1H), 4.09 (d, J=13.8 Hz, 1H), 3.29 (br s, 1H), 3.05-2.90 (m, 2H), 2.81 (br d, J=9.4 Hz, 1H), 2.72-2.58 (m, 1H), 2.51-2.34 (m, 2H), 2.03-1.88 (m, 1H), 1.41 (s, 3H).

### 2-(((S)-3-(6-((4-cyano-2-fluorobenzyl)oxy)pyridin-2-yl)-3-methylpyrrolidin-1-yl)methyl)-1-((S)-oxetan-2-ylmethyl)-1H-benzo[d]imidazole-6-carboxylic acid (Compound 20-P2)

LiOH·H2O (6.16 mg, 146.76 umol, 1.1 eq) was added to the solution of methyl 2-(((S)-3-(6-((4-cyano-2-fluorobenzyl)oxy)pyridin-2-yl)-3-methylpyrrolidin-1-yl)methyl)-1-((S)-oxetan-2-ylmethyl)-1H-benzo[d]imidazole-6-carboxylate (20j, 76 mg, 133.42 umol, 1 eq) in THF (5.6 mL) and H2O (2.4 mL) at 20° C. Then the solution was stirred at 20° C. for 24 hour. LCMS detected the desired product MS and showed that 20j remained. LiOH·H2O (6.16 mg, 146.76 umol, 1.1 eq) was added to the solution at 20° C. Then the mixture was stirred at 20° C. for another 24 hours. LCMS detected the desired product MS and showed that the 20j was consumed. The mixture was adjusted to pH=7 with HOAc. The mixture was extracted with ethyl acetate (10 mL*3). The combined organic layers were washed with brine (15 mL), dried over Na2SO4, filtered and concentrated under reduced pressure. The residue was purified by prep-HPLC (Phenomenex Gemini-NX C18 75*30 mm*3 um; mobile phase: [water (10 mM NH4HCO3)-ACN]; B %: 25%-55%, 8 min) to give 20-P2 as a white solid. MS mass calculated for [M+H]+ (C31H30FN5O4) requires m/z 556.3, LCMS found m/z 556.3; 1H NMR (400 MHz, MeOD-d4) δ 8.26 (s, 1H), 7.94 (dd, J=1.2, 8.6 Hz, 1H), 7.71-7.43 (m, 5H), 7.01 (d, J=7.4 Hz, 1H), 6.72 (d, J=8.1 Hz, 1H), 5.50-5.35 (m, 2H), 5.17-5.06 (m, 1H), 4.73 (br dd, J=7.2, 15.4 Hz, 1H), 4.62-4.51 (m, 2H), 4.42-4.18 (m, 3H), 3.41 (br d, J=9.6 Hz, 1H), 3.15-3.04 (m, 1H), 3.02-2.93 (m, 1H), 2.90 (br d, J=9.6 Hz, 1H), 2.73-2.60 (m, 1H), 2.52-2.35 (m, 2H), 2.05-1.92 (m, 1H), 1.42 (s, 3H).

When a mixture of stereoisomers is separated by HPLC, it is to be appreciated that the resultant individual stereoisomers or mixtures will be arbitrarily assigned. In the examples described herein, when the mixture of stereoisomers is separated by HPLC, it is to be appreciated that an eluting enantiomer or an enantiomer of a resulting compound prepared from the eluting enantiomer is labeled “P1” and another eluting enantiomer or an enantiomer of a resulting compound prepared from the another eluting enantiomer is labeled “P2”. In this example, the eluting enantiomers are 20i and 20j, and the resulting compound is Compound 20. The absolute configuration of the enantiomers, e.g., 20i & 20j, as well as Compounds 20-P1 & 20-P2 each associated with the corresponding 1H NMR data, may be obtained by known methods.

### Example 21 (General Procedure U) (S)-2-((4-(3-(benzyloxy)-5-methyl-1H-pyrazol-1-yl)piperidin-1-yl)methyl)-1-(oxetan-2-ylmethyl)-1H-benzo[d]imidazole-6-carboxylic acid

The title was prepared according to Scheme 17. This General Procedure V exemplifies Scheme 17 and provides particular synthetic details as applied to the title compound.

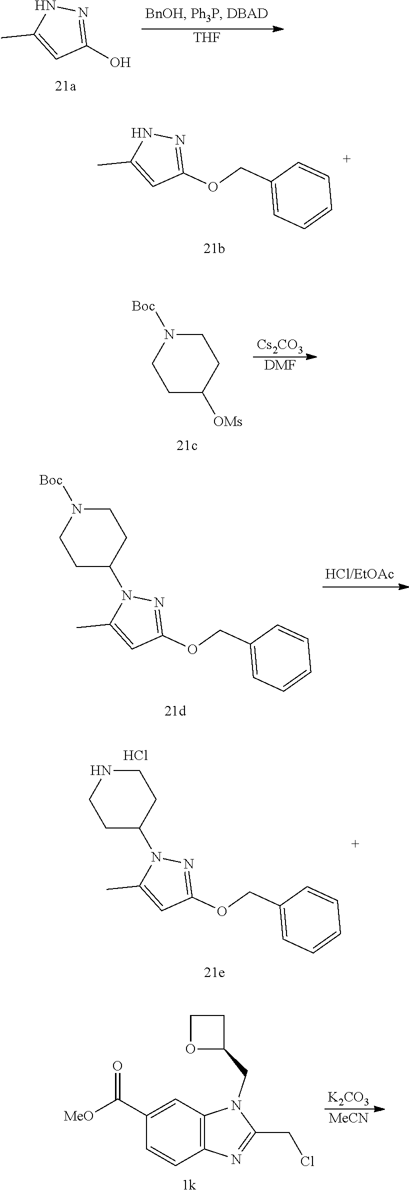

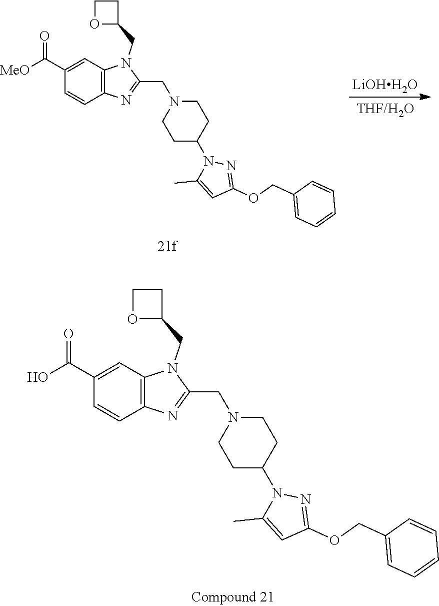 [SMILES: COC(=O)c1ccc2nc(CN3CCC(n4nc(OCc5ccccc5)cc4C)CC3)n(C[C@@H]3CCO3)c2c1 ; "LiOH·H2O" ; Cc1cc(OCc2ccccc2)nn1C1CCN(Cc2nc3ccc(C(=O)O)cc3n2C[C@@H]2CCO2)CC1]

3-(benzyloxy)-5-methyl-1H-pyrazole (21b). PPh3 (2.35 g, 8.97 mmol, 1.1 eq) was added to the solution of 5-methyl-1H-pyrazol-3-ol (21a, 0.8 g, 8.15 mmol, 1 eq) and phenylmethanol (1.59 g, 14.68 mmol, 1.53 mL, 1.8 eq) in THF (20 mL) at 20° C. Then DBAD (2.07 g, 8.97 mmol, 1.1 eq) in THF (1 mL) was added to the solution at 20° C. and the reaction was stirred at 20° C. for 16 hours. TLC (petroleum ether:ethyl acetate=1:1) showed 21a remained and one new spot was formed. The mixture was extracted with ethyl acetate (20 mL*3) and H2O (10 mL). The combined ethyl acetate was washed with brine (30 mL), dried over Na2SO4, filtered and concentrated under reduced puressure. The residue was purified by column chromatography (SiO2, petroleum ether:ethyl acetate=80:1 to 20:1) to give 21b as a light yellow solid. 1H NMR (400 MHz, MeOD-d4) δ 7.47-7.25 (m, 6H), 5.53 (s, 1H), 5.08 (s, 2H), 2.21 (s, 3H).

### Tert-butyl 4-(3-(benzyloxy)-5-methyl-1H-pyrazol-1-yl)piperidine-1-carboxylate (21d)

Cs2CO3 (1.23 g, 3.76 mmol, 2 eq) was added to the solution of 3-(benzyloxy)-5-methyl-1H-pyrazole (21b, 354 mg, 1.88 mmol, 1 eq) and tert-butyl 4-((methylsulfonyl)oxy)piperidine-1-carboxylate (21c, 840.62 mg, 3.01 mmol, 1.6 eq) in DMF (7 mL) at 20° C. Then the mixture was stirred at 100° C. for 20 hours. TLC (petroleum ether:ethyl acetate=1:1) showed trace of 21b remained and two new spots were formed. The mixture was extracted with ethyl acetate (20 mL*3) and H2O (20 mL). The combined ethyl acetate was washed with brine (15 mL), dried over Na2SO4, filtered and concentrated under reduced pressure. The residue was purified by column chromatography (SiO2, petroleum ether:ethyl acetate=80:1 to 20:1) to give 21d as colourless oil. 1H NMR (400 MHz, MeOD-d4) δ ppm 7.43-7.38 (m, 2H), 7.37-7.25 (m, 4H), 5.07 (s, 2H), 4.24-4.12 (m, 3H), 2.92 (br s, 2H), 2.24 (s, 3H), 1.96 (dq, J=4.2, 12.4 Hz, 2H), 1.79 (br d, J=12.2 Hz, 2H), 1.48 (s, 9H).

### 4-(3-(benzyloxy)-5-methyl-1H-pyrazol-1-yl)piperidine HCl salt (21e)

A solution of tert-butyl 4-(3-(benzyloxy)-5-methyl-1H-pyrazol-1-yl)piperidine-1-carboxylate (21d, 160 mg, 430.72 umol, 1 eq) in HCl/EtOAc (2 mL) was stirred at 20° C. for 0.5 hours. TLC (petroleum ether:ethyl acetate=3:1) showed 21d was consumed and one new spot was formed. The solution was concentrated. The residue was purified by prep-TLC (petroleum ether:ethyl acetate=3:1) to give 21e as a white solid. 1H NMR (400 MHz, MeOD-d4) δ 7.51-7.23 (m, 5H), 7.21-7.08 (m, 1H), 5.23-5.06 (m, 2H), 3.61-3.48 (m, 2H), 3.25-3.08 (m, 2H), 2.44-1.98 (m, 8H).

### (S)-methyl 2-((4-(3-(benzyloxy)-5-methyl-1H-pyrazol-1-yl)piperidin-1-yl)methyl)-1-(oxetan-2-ylmethyl)-1H-benzo[d]imidazole-6-carboxylate (21e)

K2CO3 (291.84 mg, 2.11 mmol, 5 eq) was added to the solution of 4-(3-(benzyloxy)-5-methyl-1H-pyrazol-1-yl)piperidine HCl salt (21e, 130 mg, 422.33 umol, 1 eq, HCl) and (S)-methyl 2-(chloromethyl)-1-(oxetan-2-ylmethyl)-1H-benzo[d]imidazole-6-carboxylate (1k, 124.47 mg, 422.33 umol, 1 eq) in CH3CN (10 mL) at 20° C. Then the solution was stirred at 50° C. for 16 hours. TLC (ethyl acetate:methanol=10:1) showed 21e was consumed and one new spot was formed. The mixture was filtered and the filtrate was concentrated under reduced pressure. The residue was purified by prep-TLC (ethyl acetate:methanol=10:1) to give 21e as a white solid. 1H NMR (400 MHz, MeOD-d4) δ 8.37 (d, J=1.0 Hz, 1H), 7.96 (dd, J=1.6, 8.6 Hz, 1H), 7.68 (d, J=8.4 Hz, 1H), 7.45-7.39 (m, 2H), 7.38-7.27 (m, 3H), 5.52 (s, 1H), 5.31-5.23 (m, 1H), 5.08 (s, 2H), 4.93 (dd, J=7.4, 15.4 Hz, 1H), 4.74 (dd, J=2.4, 15.4 Hz, 1H), 4.69-4.61 (m, 1H), 4.49 (td, J=6.0, 9.2 Hz, 1H), 4.09-4.01 (m, 2H), 3.96-3.86 (m, 3H), 3.06 (br d, J=11.8 Hz, 1H), 2.95-2.81 (m, 2H), 2.63 (td, J=1.9, 3.8 Hz, 1H), 2.59-2.49 (m, 1H), 2.42-2.07 (m, 7H), 1.80 (br t, J=13.4 Hz, 2H).

### (S)-2-((4-(3-(benzyloxy)-5-methyl-1H-pyrazol-1-yl)piperidin-1-yl)methyl)-1-(oxetan-2-ylmethyl)-1H-benzo[d]imidazole-6-carboxylic acid (Compound 21)

LiOH·H2O (47.54 mg, 1.13 mmol, 4 eq) was added to the solution of (S)-methyl 2-((4-(3-(benzyloxy)-5-methyl-1H-pyrazol-1-yl)piperidin-1-yl)methyl)-1-(oxetan-2-ylmethyl)-1H-benzo[d]imidazole-6-carboxylate (21e, 150 mg, 283.22 umol, 1 eq) in THF (7 mL) and H2O (3 mL) at 20° C. Then the solution was stirred at 20° C. for 40 hours. LCMS showed 21e was consumed and desired mass was detected. The mixture was adjusted to pH=8 with HOAc. The mixture was extracted with ethyl acetate (10 mL*3) and H2O (10 mL). The combined organic layers were washed with brine (15 mL), dried over Na2SO4, filtered and concentrated under reduced pressure. The residue was purified by prep-HPLC (column: Waters Xbridge Prep OBD C18 150*40 mm*10 um; mobile phase: [water (10 mM NH4HCO3)-ACN]; B %: 15%-50%, 8 min) to give Compound 21 as a white solid. MS mass calculated for [M+1]+(C29H33N5O4) requires m/z 516.3, LCMS found m/z 516.3. 1H NMR (400 MHz, MeOD-d4) δ 8.34 (s, 1H), 7.97 (dd, J=1.2, 8.4 Hz, 1H), 7.66 (d, J=8.4 Hz, 1H), 7.44-7.37 (m, 2H), 7.37-7.24 (m, 3H), 5.51 (s, 1H), 5.32-5.22 (m, 1H), 5.07 (s, 2H), 4.91 (dd, J=7.4, 15.4 Hz, 1H), 4.73 (dd, J=2.3, 15.4 Hz, 1H), 4.69-4.61 (m, 1H), 4.48 (td, J=6.0, 9.2 Hz, 1H), 4.10-3.99 (m, 2H), 3.98-3.89 (m, 1H), 3.08 (br d, J=11.4 Hz, 1H), 2.95 (br d, J=11.2 Hz, 1H), 2.90-2.80 (m, 1H), 2.59-2.48 (m, 1H), 2.46-2.29 (m, 2H), 2.26-2.06 (m, 5H), 1.81 (br t, J=12.4 Hz, 2H).

### Example 22 (General Procedure V)

### (S)-2-((4-(3-(benzyloxy)-1H-pyrazol-1-yl)piperidin-1-yl)methyl)-1-(oxetan-2-ylmethyl)-1H-benzo[d]imidazole-6-carboxylic acid

The title compound was prepared according to Scheme 17. This General Procedure V exemplifies Scheme 17 and provides particular synthetic details as applied to the title compound.

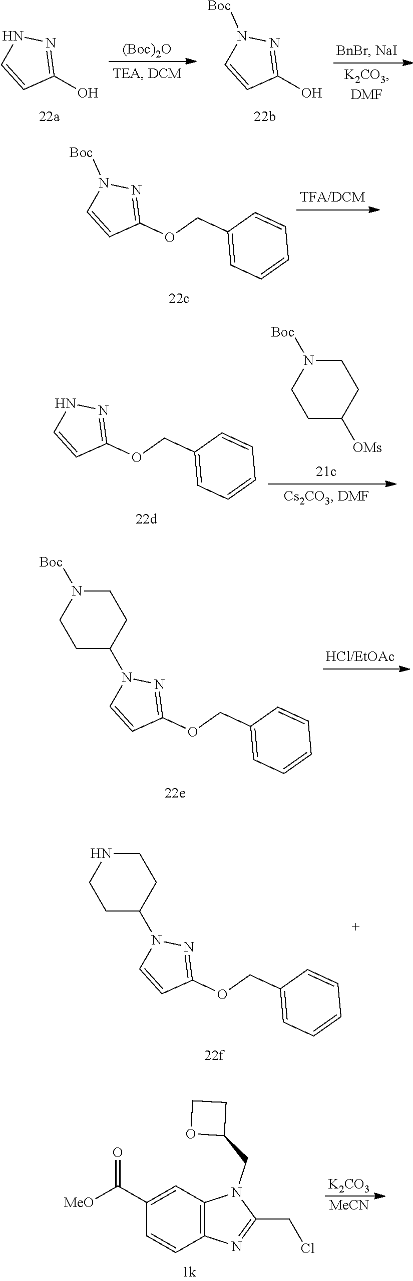

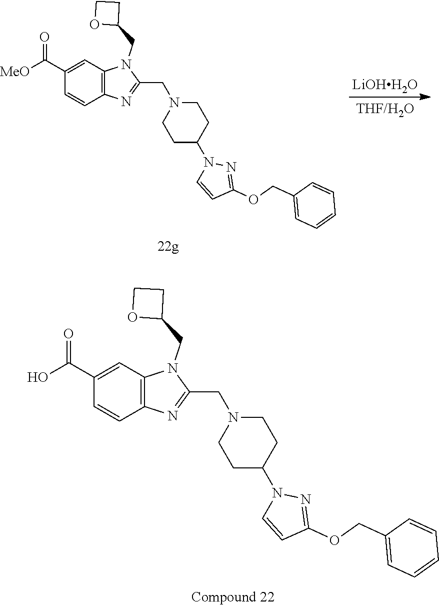 [SMILES: COC(=O)c1ccc2nc(CN3CCC(n4ccc(OCc5ccccc5)n4)CC3)n(C[C@@H]3CCO3)c2c1 ; "LiOH·H2O" ; O=C(O)c1ccc2nc(CN3CCC(n4ccc(OCc5ccccc5)n4)CC3)n(C[C@@H]3CCO3)c2c1]

### Tert-butyl 3-hydroxy-1H-pyrazole-1-carboxylate (22b)

To a solution of 1H-pyrazol-3-ol (22a, 1g, 11.89 mmol, 1 eq) in DCM (20 mL) was added (Boc)20 (2.86 g, 13.08 mmol, 3.01 mL, 1.1 eq) and TEA (1.32 g, 13.08 mmol, 1.82 mL, 1.1 eq). The mixture was stirred at 25° C. for 2 hours. LCMS showed desired mass was detected. The reaction mixture was extracted with DCM (50 mL*3) and H2O (20 mL). The combined organic layers were washed with brine (30 mL), dried over anhydrous Na2SO4, filtered and concentrated under reduced pressure. The crude product was triturated with MTBE (50 mL) at 20° C. for 15 minutes and filtered. The filter cake was dried in vacuo to give 22b as a white solid. 1H NMR (400 MHz, CDCl3-d) δ 12.36 (br s, 1H), 7.83 (br s, 1H), 5.91 (d, J=2.8 Hz, 1H), 1.73-1.57 (m, 9H).

### Tert-butyl 3-(benzyloxy)-1H-pyrazole-1-carboxylate (22c)

To a solution of tert-butyl 3-hydroxy-1H-pyrazole-1-carboxylate (22b, 400 mg, 2.17 mmol, 1 eq) and BnBr (742.85 mg, 4.34 mmol, 515.87 uL, 2 eq) in DMF (4 mL) was added NaI (325.52 mg, 2.17 mmol, 1 eq) and K2CO3 (900.40 mg, 6.51 mmol, 3 eq). The mixture was stirred at 60° C. for 16 hours. LCMS showed 22b was consumed, and one main peak with desired mass was detected. The reaction mixture was added H2O (20 mL), and then extracted with MTBE (150 mL). The organic layer was dried over anhydrous Na2SO4, filtered and concentrated under reduced pressure. The residue was purified by column chromatography (SiO2, petroleum ether:ethyl acetate=20:1 to 1:1) to give 22c as white oil. 1H NMR (400 MHz, CDCl3-d) δ 7.78 (d, J=4.0 Hz, 1H), 7.28 (br s, 1H), 7.27-7.23 (m, 2H), 7.22-7.14 (m, 2H), 5.70 (d, J=4.0 Hz, 1H), 5.34 (s, 2H), 1.51 (s, 9H).

### 3-(benzyloxy)-1H-pyrazole (22d)

To a solution of tert-butyl 3-(benzyloxy)-1H-pyrazole-1-carboxylate (22c, 200 mg, 729.09 umol, 1 eq) in DCM (1.5 mL) was added TFA (880.00 mg, 7.72 mmol, 571.43 uL, 10.59 eq). The mixture was stirred at 20° C. for 3 hours. TLC (petroleum ether:ethyl acetate=0:1) show 22c was consumed, and one new spot was generated. The reaction mixture was concentrated under reduced pressure to give 22d as a white solid. The product was used in the next step without further purification. 1H NMR (400 MHz, MeOD-d4) δ 7.36-7.22 (m, 5H), 7.18 (d, J=7.0 Hz, 2H), 5.09 (s, 2H).

### Tert-butyl 4-(3-(benzyloxy)-1H-pyrazol-1-yl)piperidine-1-carboxylate (22e)

To a solution of 3-(benzyloxy)-1H-pyrazole (22d, 110 mg, 522.17 umol, 1 eq, TFA salt) and tert-butyl 4-((methylsulfonyl)oxy)piperidine-1-carboxylate (21c, 364.67 mg, 1.31 mmol, 2.5 eq) in DMF (4 mL) was added Cs2CO3 (425.33 mg, 1.31 mmol, 2.5 eq). The mixture was stirred at 90° C. for 16 hours. LCMS showed 21c was consumed, and desired mass was detected. The reaction was filtered and extract with MTBE (150 mL), dried over anhydrous Na2SO4, filtered and concentrated under reduced pressure. The residue was purified by prep-HPLC (neutral condition; column: Waters Xbridge Prep OBD C18 150*40 mm*10 um; mobile phase: [water (10 mM NH4HCO3)-ACN]; B %: 35%-65%, 8 min) to give 22e as a white solid. 1H NMR (400 MHz, CDCl3-d) δ 7.36 (d, J=2.0 Hz, 1H), 7.34-7.27 (m, 2H), 7.27-7.23 (m, 1H), 7.22-7.18 (m, 2H), 5.51 (d, J=2.0 Hz, 1H), 5.16 (s, 2H), 4.33 (tt, J=3.4, 6.8 Hz, 1H), 3.54-3.27 (m, 4H), 1.94-1.65 (m, 4H), 1.47 (s, 9H).

### 4-(3-(benzyloxy)-1H-pyrazol-1-yl)piperidine (22f)

A mixture of tert-butyl 4-(3-(benzyloxy)-1H-pyrazol-1-yl)piperidine-1-carboxylate (22e, 79 mg, 221.01 umol, 1 eq) in HCl/EtOAc (4 M, 7.90 mL) was stirred at 20° C. for 1 hour under N2 atmosphere. LCMS showed 22e was consumed, and desired mass was detected. The reaction mixture was concentrated under reduced pressure to give 22f as a white solid. The mixture was used to next step without purification. 1H NMR (400 MHz, MeOD-d4) δ 8.05-7.85 (m, 1H), 7.46-7.33 (m, 3H), 7.25 (br d, J=7.6 Hz, 2H), 6.34-6.21 (m, 1H), 5.40 (d, J=4.0 Hz, 2H), 4.65-4.81 (m, 1H), 3.25-3.16 (m, 4H), 2.31-2.03 (m, 4H).

### (S)-methyl 2-((4-(3-(benzyloxy)-1H-pyrazol-1-yl)piperidin-1-yl)methyl)-1-(oxetan-2-ylmethyl)-1H-benzo[d]imidazole-6-carboxylate (22g)

To a solution of 4-(3-(benzyloxy)-1H-pyrazol-1-yl)piperidine (22f, 64 mg, 217.84 umol, 1 eq, HCl), (S)-methyl 2-(chloromethyl)-1-(oxetan-2-ylmethyl)-1H-benzo[d]imidazole-6-carboxylate (1k, 70.63 mg, 239.63 umol, 1.1 eq) in ACN (2 mL) was added K2CO3 (150.54 mg, 1.09 mmol, 5 eq). The mixture was stirred at 50° C. for 16 hours. LCMS showed 22f was consumed, and desired mass was detected. The reaction mixture was filtered and concentrated under reduced pressure. The residue was purified by prep-TLC (SiO₂, ethyl acetate:methanol=10:1) to give 22g as a white solid. 1H NMR (400 MHz, MeOD-d4) δ 8.33 (s, 1H), 7.96 (dd, J=1.3, 8.6 Hz, 1H), 7.67 (d, J=8.4 Hz, 1H), 7.36-7.23 (m, 4H), 7.16 (d, J=7.0 Hz, 2H), 5.70 (d, J=2.2 Hz, 1H), 5.26-5.18 (m, 1H), 5.16 (s, 2H), 4.74-4.54 (m, 2H), 4.50-4.33 (m, 2H), 4.00-3.90 (m, 4H), 3.89-3.79 (m, 1H), 2.86-2.70 (m, 2H), 2.68-2.33 (m, 5H), 2.06-1.70 (m, 4H).

### (S)-2-((4-(3-(benzyloxy)-1H-pyrazol-1-yl)piperidin-1-yl)methyl)-1-(oxetan-2-ylmethyl)-1H-benzo[d]imidazole-6-carboxylic acid (22)

To a solution of (S)-methyl 2-((4-(3-(benzyloxy)-1H-pyrazol-1-yl)piperidin-1-yl)methyl)-1-(oxetan-2-ylmethyl)-1H-benzo[d]imidazole-6-carboxylate (22g, 90 mg, 174.55 umol, 1 eq) in THF (3.15 mL) and H2O (1.35 mL) was added LiOH·H2O (25 mg, 595.75 umol, 3.41 eq). The mixture was stirred at 20° C. for 16 hours. LCMS showed 22g was consumed, and desired mass was detected. The reaction mixture was added citric acid until pH=6 and filtered. The filtrate was concentrated under reduced pressure. The crude product was triturated with H2O (1.5 mL) and filtered. The filter cake was dried over under reduced pressure to give Compound 22 as a white solid. MS mass calculated for [M+1]+(C28H31N5O4) requires m/z 502.2, LCMS found m/z 502.3. 1H NMR (400 MHz, MeOD-d4) δ8.32 (s, 1H), 7.97 (br d, J=8.3 Hz, 1H), 7.66 (d, J=8.4 Hz, 1H), 7.37-7.23 (m, 4H), 7.16 (br d, J=7.6 Hz, 2H), 5.71 (s, 1H), 5.22 (br d, J=6.8 Hz, 1H), 5.16 (s, 2H), 4.82 (br d, J=7.2 Hz, 1H), 4.74-4.59 (m, 2H), 4.49-4.33 (m, 2H), 4.05-3.78 (m, 2H), 2.87-2.71 (m, 1H), 2.62 (br s, 2H), 2.55-2.41 (m, 3H), 1.98 (br s, 2H), 1.81 (br s, 2H).

### Example 23 (General Procedure W)

### (S)-2-((4-(3-(benzyloxy)-4,5,6,7-tetrahydro-1H-indazol-1-yl)piperidin-1-yl)methyl)-1-(oxetan-2-ylmethyl)-1H-benzo[d]imidazole-6-carboxylic acid

The title compound was prepared according to Scheme 18. This General Procedure W exemplifies Scheme 18 and provides particular synthetic details as applied to the title compound.

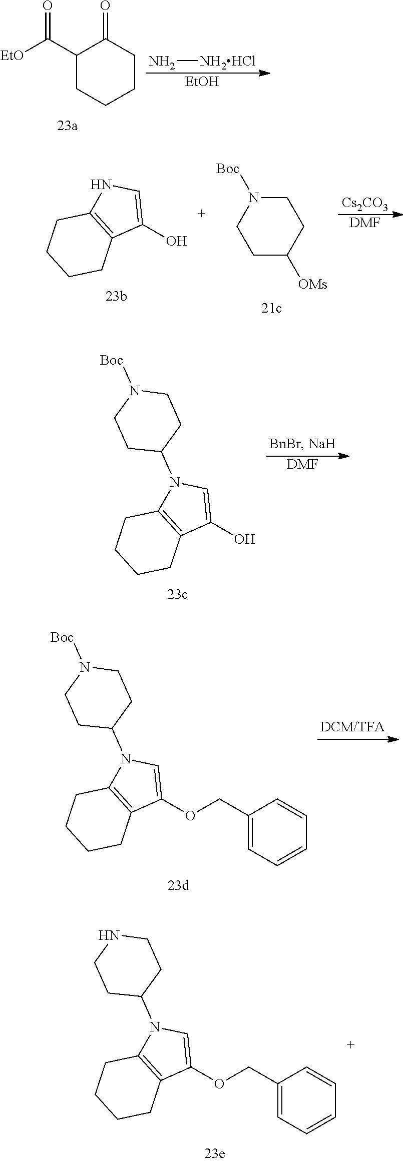

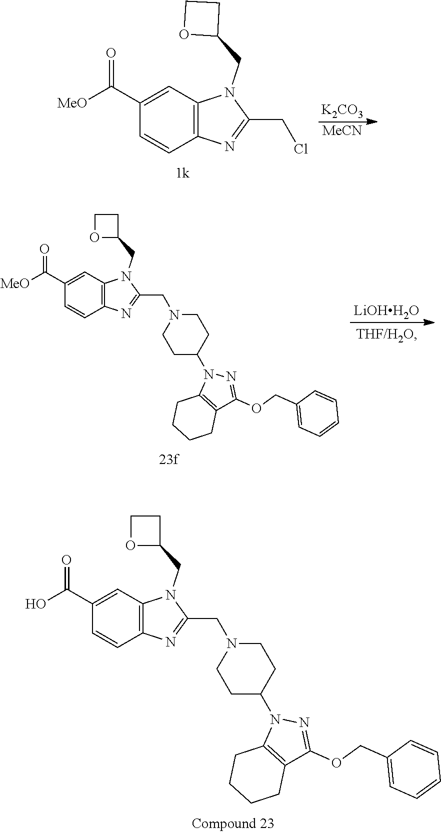

### 4,5,6,7-tetrahydro-1H-indazol-3-ol (23b)

To a solution of ethyl 2-oxocyclohexanecarboxylate (23a, 1g, 5.88 mmol, 943.40 uL, 1 eq) in EtOH (10 mL) was added NH2-NH2·H2O (450.18 mg, 8.81 mmol, 437.06 uL, 98% purity, 1.5 eq). The mixture was stirred at 20° C. for 16 hours. LCMS showed 23a was consumed and desired mass was detected. The reaction mixture was cooled to room temperature and stirred for 10 minutes. Then the white solid was collected by filtration. The aqueous phase was quenched with HCl (1 M, 2 mL) and discard. The filter cake was washed with EtOH (3 mL*3) and drieded under reduced pressure to give 23b as a white solid. 1H NMR (400 MHz, MeOD-d4) δ 2.49 (t, J=6.0 Hz, 2H), 2.30 (t, J=5.8 Hz, 2H), 1.84-1.67 (m, 4H).

### Tert-butyl 4-(3-hydroxy-4,5,6,7-tetrahydro-1H-indazol-1-yl)piperidine-1-carboxylate (23c)

To a solution of 4,5,6,7-tetrahydro-1H-indazol-3-ol (23b, 600 mg, 4.34 mmol, 1 eq) and tert-butyl 4-((methylsulfonyl)oxy)piperidine-1-carboxylate (21c, 1.33 g, 4.78 mmol, 1.1 eq) in DMF (10 mL) was added Cs2CO3 (2.83 g, 8.69 mmol, 2 eq). The mixture was stirred at 80° C. for 16 hours. LCMS showed 23b was consumed and desired mass was detected. The mixture was extracted with ethyl acetate (20 mL*3). The combined ethyl acetate was washed with brine (20 mL), dried over anhydrous Na2SO4, filtered and concentrated. The residue was purified by column chromatography (SiO2, petroleum ether:ethyl acetate=10:1 to 0:1) to give 23c as a white solid. 1H NMR (400 MHz, CDCl3-d) δ 4.74 (tt, J=3.8, 7.6 Hz, 1H), 3.83-3.67 (m, 2H), 3.32-3.22 (m, 2H), 2.55 (t, J=6.0 Hz, 2H), 2.36 (t, J=6.0 Hz, 2H), 2.02-1.84 (m, 2H), 1.83-1.67 (m, 6H), 1.47 (s, 9H).

### Tert-butyl 4-(3-(benzyloxy)-4,5,6,7-tetrahydro-1H-indazol-1-yl)piperidine-1-carboxylate (23d)

To a solution of tert-butyl 4-(3-hydroxy-4,5,6,7-tetrahydro-1H-indazol-1-yl)piperidine-1-carboxylate (23c, 470 mg, 1.46 mmol, 1 eq) in DMF (5 mL) was added NaH (87.73 mg, 2.19 mmol, 60% purity, 1.5 eq) at 0° C. The mixture was stirred 1 hour at 20° C. Then bromomethylbenzene (250.10 mg, 1.46 mmol, 173.68 uL, 1 eq) was added in the mixture. The mixture was stirred at 20° C. for another 1 hour. TLC (petroleum ether:ethyl acetate=2:1) indicated most 23c was consumed, and one major new spot was formed. The reaction mixture was quenched by addition of NH4Cl (10 mL) at 20° C., and then diluted with water (10 mL) and extracted with EtOAc (20 mL*2). The combined organic layers were dried over anhydrous Na2SO4, filtered and concentrated under reduced pressure. The residue was purified by column chromatography (SiO2, petroleum ether:ethyl acetate=10:1 to 1:1) to give 23d as a white solid. 1H NMR (400 MHz, CDCl3-d) δ 7.34-7.22 (m, 3H), 7.07 (d, J=7.0 Hz, 2H), 5.06 (s, 2H), 4.76 (td, J=3.8, 7.6 Hz, 1H), 3.80-3.66 (m, 2H), 3.34-3.23 (m, 2H), 2.38 (td, J=6.0, 12.4 Hz, 4H), 2.02-1.91 (m, 2H), 1.80-1.65 (m, 6H), 1.47 (s, 9H).

### 3-(benzyloxy)-1-(piperidin-4-yl)-4,5,6,7-tetrahydro-1H-indazole (23e)

To a solution of tert-butyl 4-(3-(benzyloxy)-4,5,6,7-tetrahydro-1H-indazol-1-yl)piperidine-1-carboxylate (23d, 320 mg, 777.58 umol, 1 eq) in DCM (5 mL) was added TFA (0.5 mL). The mixture was stirred at 20° C. for 2 hours. TLC (petroleum ether:ethyl acetate=3:1) indicated 23d was consumed, and one major new spot was formed. The mixture was adjusted to pH 8 with saturated Na2CO3 (aq). The reaction mixture was extracted with DCM (20 mL*3) and H2O (10 mL). The combined organic layers were washed with brine (20 mL), dried over anhydrous Na2SO4, filtered and concentrated under reduced pressure to give 23e as a white solid. The crude product was used in next step without any further purification. 1H NMR (400 MHz, CDCl3-d) δ 7.34-7.23 (m, 3H), 7.07 (br d, J=7.1 Hz, 2H), 5.04 (s, 2H), 4.91 (br s, 1H), 3.42-3.30 (m, 2H), 3.15 (br d, J=12.0 Hz, 2H), 2.46-2.32 (m, 4H), 2.17 (br s, 4H), 1.82-1.64 (m, 4H).

### (S)-methyl 2-((4-(3-(benzyloxy)-4,5,6,7-tetrahydro-1H-indazol-1-yl)piperidin-1-yl)methyl)-1-(oxetan-2-ylmethyl)-1H-benzo[d]imidazole-6-carboxylate (23f)

To a solution of 3-(benzyloxy)-1-(piperidin-4-yl)-4,5,6,7-tetrahydro-1H-indazole (23e, 250 mg, 802.77 umol, 1 eq) and (S)-methyl 2-(chloromethyl)-1-(oxetan-2-ylmethyl)-1H-benzo[d]imidazole-6-carboxylate (1k, 236.60 mg, 802.77 umol, 1 eq) in MeCN (5 mL) was added K2CO3 (110.95 mg, 802.77 umol, 1 eq). The mixture was stirred at 50° C. for 16 hours. LCMS showed 23e was consumed and desired mass was detected. The reaction mixture was extracted with ethyl acetate (20 mL*3) and H2O (10 mL). The combined organic layers were washed with brine (20 mL), dried over anhydrous Na2SO4, filtered and concentrated under reduced pressure. The residue was purified by column chromatography (SiO2, petroleum ether:ethyl acetate=10:1 to 0:1) to give 23f as a white solid. 1H NMR (400 MHz, MeOD-d4) δ 8.33 (s, 1H), 7.95 (dd, J=1.2, 8.6 Hz, 1H), 7.67 (d, J=8.6 Hz, 1H), 7.31-7.25 (m, 2H), 7.25-7.19 (m, 1H), 7.03 (d, J=7.0 Hz, 2H), 5.24 (dq, J=2.4, 7.2 Hz, 1H), 5.07 (s, 2H), 4.91-4.85 (m, 1H), 4.74-4.59 (m, 2H), 4.53 (td, J=3.8, 7.6 Hz, 1H), 4.45 (td, J=6.0, 9.0 Hz, 1H), 4.04-3.97 (m, 1H), 3.93 (s, 3H), 3.91-3.86 (m, 1H), 2.87-2.73 (m, 3H), 2.56-2.48 (m, 1H), 2.47-2.39 (m, 4H), 2.35 (t, J=6.0 Hz, 2H), 2.05-1.96 (m, 2H), 1.84-1.64 (m, 6H).

### (S)-2-((4-(3-(benzyloxy)-4,5,6,7-tetrahydro-1H-indazol-1-yl)piperidin-1-yl)methyl)-1-(oxetan-2-ylmethyl)-1H-benzo[d]imidazole-6-carboxylic acid (Compound 23)

To a solution of (S)-methyl 2-((4-(3-(benzyloxy)-4,5,6,7-tetrahydro-1H-indazol-1-yl)piperidin-1-yl)methyl)-1-(oxetan-2-ylmethyl)-1H-benzo[d]imidazole-6-carboxylate (23f, 150 mg, 263.30 umol, 1 eq) in THF (5 mL) and H2O (2 mL) was added LiOH·H2O (27.62 mg, 658.25 umol, 2.5 eq). The mixture was stirred at 20° C. for 16 hours. LCMS showed 23f was consumed and desired mass was detected. The mixture was adjusted to pH 6 with Citric acid (aq, 1 M) and concentrated under reduced pressure. The residue was extracted with ethyl acetate (20 mL*3) and H2O (10 mL). The combined organic layers were washed with brine (20 mL), dried over anhydrous Na2SO4, filtered and concentrated under reduced pressure to give Compound 23 as a white solid. MS mass calculated for [M+1]+(C32H37N5O4) requires m/z 556.3, LCMS found m/z 556.3. 1H NMR (400 MHz, CDCl3-d) δ 8.13-8.01 (m, 2H), 7.80 (d, J=8.4 Hz, 1H), 7.34-7.20 (m, 3H), 7.06 (br d, J=7.2 Hz, 2H), 5.18 (br d, J=4.0 Hz, 1H), 5.06 (s, 2H), 4.79-4.57 (m, 4H), 4.46-4.36 (m, 1H), 4.04 (br s, 2H), 2.90 (br s, 2H), 2.80-2.64 (m, 1H), 2.59 (br s, 2H), 2.48-2.31 (m, 5H), 2.09 (br s, 2H), 1.94-1.81 (m, 2H), 1.80-1.63 (m, 4H).

### Example 24 (General Procedure X)

### (S)-2-((4-(3-(benzyloxy)-4-methyl-1H-pyrazol-1-yl)piperidin-1-yl)methyl)-1-(oxetan-2-ylmethyl)-1H-benzo[d]imidazole-6-carboxylic acid

The title compound was prepared according to Scheme 19. This General Procedure X exemplifies Scheme 19 and provides particular synthetic details as applied to the title compound.

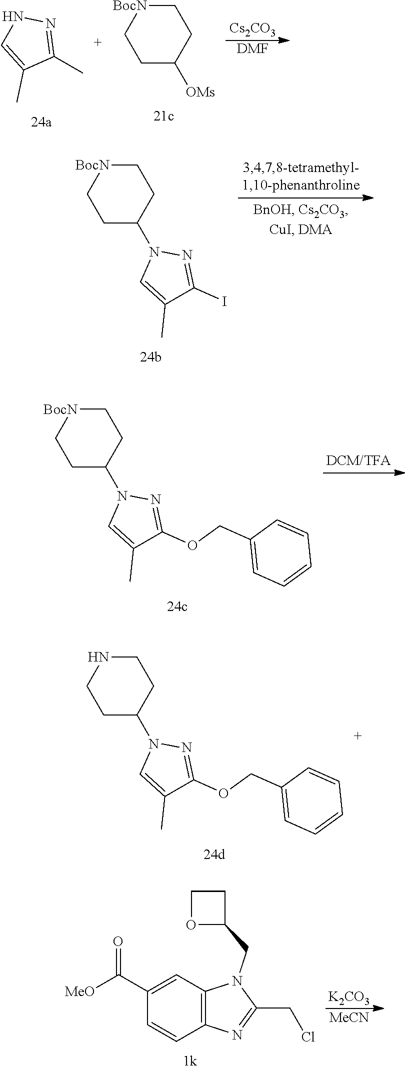

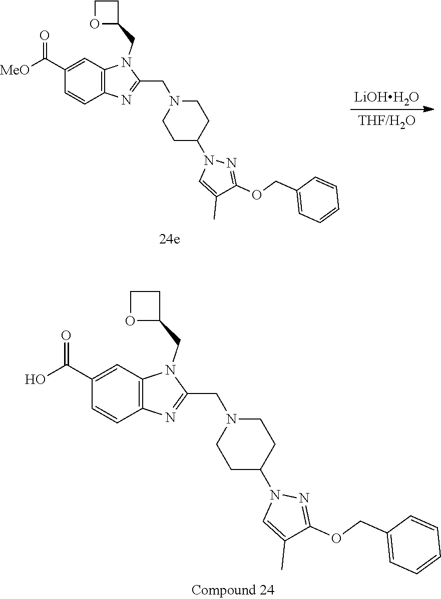 [SMILES: COC(=O)c1ccc2nc(CN3CCC(n4cc(C)c(OCc5ccccc5)n4)CC3)n(C[C@@H]3CCO3)c2c1 ; "LiOH·H2O" ; Cc1cn(C2CCN(Cc3nc4ccc(C(=O)O)cc4n3C[C@@H]3CCO3)CC2)nc1OCc1ccccc1]

Tert-butyl 4-(3-iodo-4-methyl-1H-pyrazol-1-yl)piperidine-1-carboxylate (24b).

To a solution of 3-iodo-4-methyl-1H-pyrazole (24a, 500 mg, 2.40 mmol, 1 eq) and tert-butyl 4-((methylsulfonyl)oxy)piperidine-1-carboxylate (21c, 1.34 g, 4.81 mmol, 2 eq) in DMF (10 mL) was added Cs2CO3 (1.96 g, 6.01 mmol, 2.5 eq). The mixture was stirred at 80° C. for 16 hours. TLC (petroleum ether:ethyl acetate=3:1) indicated 24a was consumed, and one major new spot was formed. The reaction mixture was extracted with ethyl acetate (30 mL*3) and H2O (30 mL). The combined organic layers were washed with brine (20 mL), dried over anhydrous Na2SO4, filtered and concentrated under reduced pressure. The residue was purified by column chromatography (SiO2, petroleum ether:ethyl acetate=10:1 to 1:1) to give 24b as a white solid. 1H NMR (400 MHz, CDCl3-d) δ 7.11 (s, 1H), 4.36-4.12 (m, 3H), 2.85 (br s, 2H), 2.12-2.04 (m, 2H), 1.99 (s, 3H), 1.93-1.81 (m, 2H), 1.47 (s, 9H).

### Tert-butyl 4-(3-(benzyloxy)-4-methyl-1H-pyrazol-1-yl)piperidine-1-carboxylate (24c)

A mixture of tert-butyl 4-(3-iodo-4-methyl-1H-pyrazol-1-yl)piperidine-1-carboxylate (24b, 180 mg, 460.07 umol, 1 eq), BnOH (497.51 mg, 4.60 mmol, 478.37 uL, 10 eq), Cs2CO3 (299.80 mg, 920.14 umol, 2 eq), CuI (17.52 mg, 92.01 umol, 0.2 eq) and 3,4,7,8-tetramethyl-1,10-phenanthroline (43.49 mg, 184.03 umol, 0.4 eq) in DMA (3 mL) was degassed and purged with N2 3 times, and then the mixture was stirred at 120° C. for 16 hours under N2 atmosphere. LCMS showed 24b was consumed and desired mass was detected. The reaction mixture was extracted with ethyl acetate (30 mL*3) and H2O (30 mL). The combined organic layers were washed with brine (20 mL), dried over anhydrous Na2SO4, filtered and concentrated under reduced pressure. The residue was purified by prep-TLC (SiO2, petroleum ether:ethyl acetate=3:1) to give 24c as colorless oil. 1H NMR (400 MHz, CDCl3-d) δ 7.47 (d, J=7.2 Hz, 2H), 7.41-7.35 (m, 2H), 7.34-7.29 (m, 1H), 7.02 (s, 1H), 5.22 (s, 2H), 4.21 (br s, 2H), 4.02 (tt, J=3.8, 11.6 Hz, 1H), 2.87 (br t, J=11.6 Hz, 2H), 2.07 (br d, J=14.2 Hz, 2H), 1.94 (s, 3H), 1.88-1.76 (m, 2H), 1.57-1.47 (m, 9H).

### 4-(3-(benzyloxy)-4-methyl-1H-pyrazol-1-yl)piperidine (24d)

To a solution of tert-butyl 4-(3-(benzyloxy)-4-methyl-1H-pyrazol-1-yl)piperidine-1-carboxylate (24c, 120 mg, 323.04 umol, 1 eq) in DCM (1 mL) was added TFA (0.1 mL). The mixture was stirred at 20° C. for 2 hours. LCMS showed 24c was consumed and desired mass was detected. The mixture was adjusted to pH=8 with saturated NaHCO3 (aq) and extracted with DCM (10 mL*2). The combined organic layers were washed with brine (5 mL), dried over anhydrous Na2SO4, filtered and centrated under reduced pressure to give 24d as colorless oil. The crude product was used in next step without further purification. 1H NMR (400 MHz, CDCl3-d) δ 7.47 (d, J=7.4 Hz, 2H), 7.41-7.29 (m, 3H), 7.05 (s, 1H), 5.22 (s, 2H), 4.09-3.98 (m, 1H), 3.31 (br d, J=12.8 Hz, 2H), 2.90-2.77 (m, 2H), 2.16 (br d, J=10.6 Hz, 2H), 2.01-1.82 (m, 5H).

### (S)-methyl 2-((4-(3-(benzyloxy)-4-methyl-1H-pyrazol-1-yl)piperidin-1-yl)methyl)-1-(oxetan-2-ylmethyl)-1H-benzo[d]imidazole-6-carboxylate (24e)

To a solution of 4-(3-(benzyloxy)-4-methyl-1H-pyrazol-1-yl)piperidine (24d, 90 mg, 331.67 umol, 1 eq) and (S)-methyl 2-(chloromethyl)-1-(oxetan-2-ylmethyl)-1H-benzo[d]imidazole-6-carboxylate (1k, 97.75 mg, 331.67 umol, 1 eq) in CH3CN (1.5 mL) was added K2CO3 (45.84 mg, 331.67 umol, 1 eq). The mixture was stirred at 50° C. for 16 hours. LCMS showed 24d was consumed and desired mass was detected. The reaction mixture was extracted with EtOAc (20 mL). The organic layer was dried over anhydrous Na2SO4, filtered and concentrated under reduced pressure. The residue was purified by prep-TLC (SiO2, ethyl acetate:methanol=5:1) to give 24e as a white solid. 1H NMR (400 MHz, CDCl3-d) δ 8.17 (s, 1H), 7.98 (dd, J=1.2, 8.6 Hz, 1H), 7.76 (d, J=8.6 Hz, 1H), 7.46 (d, J=7.4 Hz, 2H), 7.39-7.29 (m, 3H), 7.03 (s, 1H), 5.25-5.20 (m, 3H), 4.78-4.61 (m, 3H), 4.39 (td, J=6.0, 9.2 Hz, 1H), 4.03-3.89 (m, 5H), 3.04-2.95 (m, 2H), 2.81-2.72 (m, 1H), 2.51-2.41 (m, 1H), 2.32 (q, J=11.4 Hz, 2H), 2.14-2.03 (m, 2H), 1.99-1.89 (m, 5H).

### (S)-2-((4-(3-(benzyloxy)-4-methyl-1H-pyrazol-1-yl)piperidin-1-yl)methyl)-1-(oxetan-2-ylmethyl)-1H-benzo[d]imidazole-6-carboxylic acid (Compound 24)

To a solution of (S)-methyl 2-((4-(3-(benzyloxy)-4-methyl-1H-pyrazol-1-yl)piperidin-1-yl)methyl)-1-(oxetan-2-ylmethyl)-1H-benzo[d]imidazole-6-carboxylate (24e, 100 mg, 188.81 umol, 1 eq) in THF (2.5 mL) and H2O (1 mL) was added LiOH·H2O (19.81 mg, 472.03 umol, 2.5 eq). The mixture was stirred at 20° C. for 16 hours. LCMS showed 24e was consumed and desired mass was detected. The mixture was adjusted to pH=6 with citric acid (aq, 1 M), and concentrated under reduced pressure. The residue was extracted with ethyl acetate (20 mL) and water (10 mL). The combined organic layers were dried over anhydrous Na2SO4, filtered and concentrated under reduced pressure. The crude product was purified by prep-HPLC (Waters Xbridge Prep OBD C18 150*40 mm*10 um; mobile phase: [water (10 mM NH4HCO3)-ACN]; B %: 20%-50%, 8 min) to give Compound 24 as a white solid. 1H NMR (400 MHz, CDCl3-d) δ 8.19 (s, 1H), 8.06 (br d, J=8.2 Hz, 1H), 7.83 (br d, J=8.4 Hz, 1H), 7.46 (d, J=7.2 Hz, 2H), 7.39-7.28 (m, 3H), 7.03 (s, 1H), 5.22 (s, 3H), 4.79-4.60 (m, 3H), 4.40 (td, J=5.8, 9.0 Hz, 1H), 4.04 (br s, 2H), 4.00-3.88 (m, 1H), 3.05 (br t, J=12.6 Hz, 2H), 2.81-2.71 (m, 1H), 2.51-2.31 (m, 3H), 2.15-2.05 (m, 2H), 2.02-1.88 (m, 5H).

### Example 25 (General Procedure Y)

### (S)-2-((4-(3-(7-cyano-3,4-dihydroisoquinolin-2(1H)-yl)-1H-pyrazol-1-yl)piperidin-1-yl)methyl)-1-(oxetan-2-ylmethyl)-1H-benzo[d]imidazole-6-carboxylic acid

The title compound was prepared according to Scheme 19. This General Procedure Y exemplifies Scheme 19 and provides particular synthetic details as applied to the title compound.

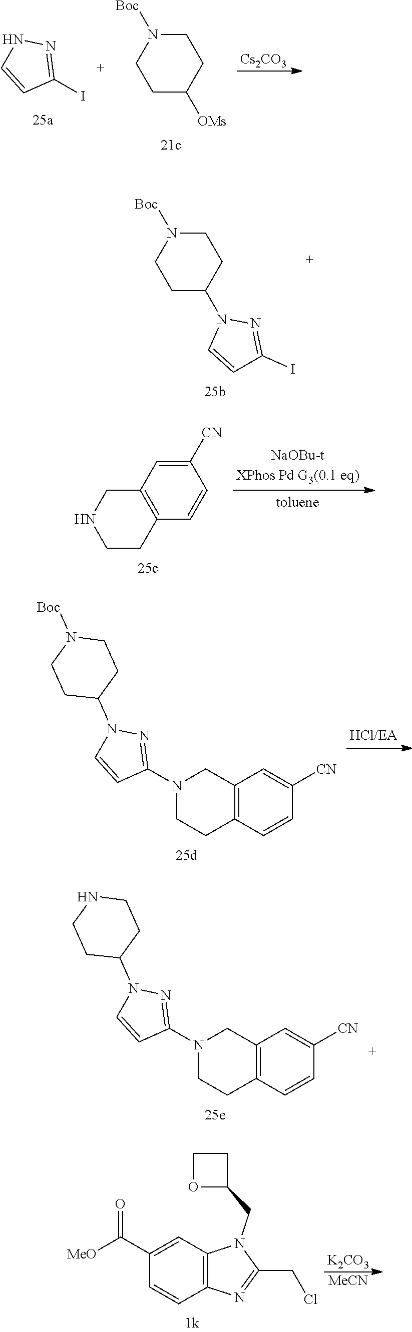

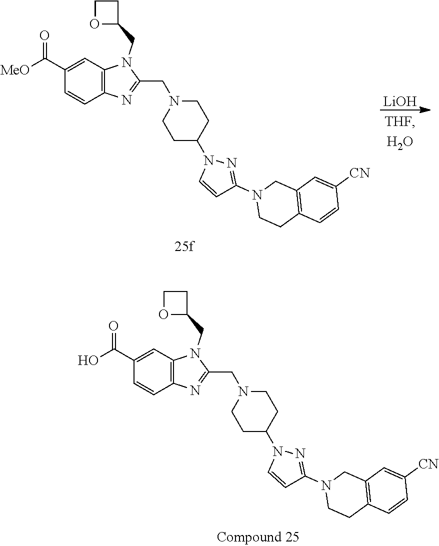 [SMILES: COC(=O)c1ccc2nc(CN3CCC(n4ccc(N5CCc6ccc(C#N)cc6C5)n4)CC3)n(C[C@@H]3CCO3)c2c1 ; "LiOH" ; N#Cc1ccc2c(c1)CN(c1ccn(C3CCN(Cc4nc5ccc(C(=O)O)cc5n4C[C@@H]4CCO4)CC3)n1)CC2]

### Tert-butyl 4-(3-iodo-1H-pyrazol-1-yl)piperidine-1-carboxylate (25b)

To a solution of 3-iodo-1H-pyrazole (25a, 4.97 g, 25.62 mmol, 1 eq) and tert-butyl 4-methylsulfonyloxypiperidine-1-carboxylate (21c, 13.6 g, 48.68 mmol, 1.9 eq) in DMF (140 mL) was added Cs2CO3 (20.87 g, 64.06 mmol, 2.5 eq) at 20° C. under N2. The mixture was stirred at 80° C. for 24 hours. LCMS showed 25a was consumed completely and desired mass was detected. The reaction mixture was extracted with ethyl acetate (500 mL) and H2O (200 mL*3). The combined organic layers were dried over anhydrous Na2SO4, filtered and concentrated under reduced pressure. The residue was purified by prep-TLC (SiO2, petroleum ether:ethyl acetate=5:1 to 3:1) to give 25b as a white solid. 1H NMR (400 MHz, CDCl3-d) δ 7.24 (d, J=2.4 Hz, 1H), 6.39 (d, J=2.4 Hz, 1H), 4.32-4.16 (m, 3H), 2.83 (br t, J=11.6 Hz, 2H), 2.13-2.04 (m, 2H), 1.87 (dq, J=4.4, 12.4 Hz, 2H), 1.45 (s, 9H).

### Tert-butyl 4-(3-(7-cyano-3,4-dihydroisoquinolin-2(1H)-yl)-1H-pyrazol-1-yl)piperidine-1-carboxylate (25d)

To a solution of tert-butyl 4-(3-iodo-1H-pyrazol-1-yl)piperidine-1-carboxylate (25b, 119.22 mg, 316.06 umol, 1 eq) and 1,2,3,4-tetrahydroisoquinoline-7-carbonitrile (25c, 50.00 mg, 316.06 umol, 1 eq) in toluene (2 mL) was added NaOtBu (60.75 mg, 632.11 umol, 2 eq) and XPhos Pd G3 (26.75 mg, 31.61 umol, 0.1 eq) under N2. The mixture was stirred at 100° C. for 16 hours under N2. LCMS showed 25b was consumed and desired mass was detected. The mixture was diluted with H2O (15 mL) and extracted with ethyl acetate (20 mL *2). The combined organic layers were washed with brine (10 mL*3), dried over with anhydrous Na2SO4, filtered and concentrated under reduced pressure. The residue was purified by prep-TLC (petroleum ether:ethyl acetate=1:1) to give 25d as a yellow solid. 1H NMR (400 MHz, CDCl3-d) δ 7.40-7.33 (m, 1H), 7.18-7.13 (m, 1H), 5.68 (d, J=2.4 Hz, 1H), 4.33 (s, 1H), 4.28-4.10 (m, 2H), 4.09-3.99 (m, 2H), 3.46 (t, J=5.8 Hz, 2H), 2.95 (t, J=5.6 Hz, 2H), 2.81 (br t, J=12.0 Hz, 2H), 2.13-2.00 (m, 2H), 1.76 (br dd, J=4.0, 12.2 Hz, 2H), 1.44-1.34 (m, 9H).

### 2-(1-(piperidin-4-yl)-1H-pyrazol-3-yl)-1,2,3,4-tetrahydroisoquinoline-7-carbonitrile (25e)

A solution of tert-butyl 4-(3-(7-cyano-3,4-dihydroisoquinolin-2(1H)-yl)-1H-pyrazol-1-yl)piperidine-1-carboxylate (25d, 30 mg, 73.62 umol, 1 eq) in HCl/EtOAc (1 mL) was stirred at 25° C. for 1 hour. LCMS showed 25d was consumed completely and desired mass was detected. The mixture was concentrated in vacuo to give 25e as a white solid. The solid was used directly for the next step without further purification.

### (S)-methyl 2-((4-(3-(7-cyano-3,4-dihydroisoquinolin-2(1H)-yl)-1H-pyrazol-1-yl)piperidin-1-yl)methyl)-1-(oxetan-2-ylmethyl)-1H-benzo[d]imidazole-6-carboxylate (25f)

To a mixture of 2-(1-(piperidin-4-yl)-1H-pyrazol-3-yl)-1,2,3,4-tetrahydroisoquinoline-7-carbonitrile (25e, 25 mg, 72.71 umol, 1 eq, HCl salt) in CH3CN (2 mL) was added K2CO3 (40.19 mg, 290.82 umol, 4 eq). The mixture was stirred at 20° C. for 0.5 hour under N2. Then (S)-methyl 2-(chloromethyl)-1-(oxetan-2-ylmethyl)-1H-benzo[d]imidazole-6-carboxylate (1k, 23.57 mg, 79.98 umol, 1.1 eq) was added in the reaction mixture. The mixture was stirred at 50° C. for 15.5 hours. LCMS showed 25e was comassumed completely and desired mass was detected. The mixture was diluted with H2O (10 mL) and extracted with ethyl acetate (30 mL *2). The combined organic layers were washed with brine (15 mL*3), dried over with anhydrous Na2SO4, filtered and concentrated under reduced pressure. The residue was purified by prep-TLC (ethyl acetate:Methanol=10:1) to give 25f as a yellow solid. The product was used directly in next step with out any further purification.

### (S)-2-((4-(3-(7-cyano-3,4-dihydroisoquinolin-2(1H)-yl)-1H-pyrazol-1-yl)piperidin-1-yl)methyl)-1-(oxetan-2-ylmethyl)-1H-benzo[d]imidazole-6-carboxylic acid (Compound 25)

To a solution of (S)-methyl 2-((4-(3-(7-cyano-3,4-dihydroisoquinolin-2(1H)-yl)-1H-pyrazol-1-yl)piperidin-1-yl)methyl)-1-(oxetan-2-ylmethyl)-1H-benzo[d]imidazole-6-carboxylate (25f, 10 mg, 17.68 umol, 1 eq) in THF (0.7 mL) and H2O (0.3 mL) was added LiOH·H2O (1.48 mg, 35.36 umol, 75.67 uL, 2 eq) at 25° C. The mixture was stirred at 25° C. for 16 hours. LCMS showed 25f was comassumed completely and desired mass was detected. The mixture was adjusted to pH=6 with citric acid (1 M), and extracted with ethyl acetate (20 mL *2) and H2O (15 mL). The combined organic layers were washed with brine (10 mL*3), dried over with anhydrous Na2SO4, filtered and concentrated under reduced pressure. The residue was purified by Prep-HPLC (NH4HCO3) column: Waters Xbridge BEH C18 100*30 mm*10 um; mobile phase: [water (10 mM NH4HCO3)-ACN]; B %: 10%-40%, 8 min) to give Compound 25 as a white solid. MS mass calculated for [M+1]+(C31H33N7O3) requires m/z 552.3, LCMS found m/z 552.3. 1H NMR (400 MHz, CDCl3-d) δ 8.19 (s, 1H), 8.04 (dd, J=1.2, 8.4 Hz, 1H), 7.81 (d, J=8.4 Hz, 1H), 7.48-7.39 (m, 2H), 7.28 (d, J=2.4 Hz, 1H), 7.23 (d, J=7.8 Hz, 1H), 5.74 (d, J=2.4 Hz, 1H), 5.28-5.17 (m, 1H), 4.80-4.60 (m, 3H), 4.46-4.35 (m, 3H), 4.02 (s, 3H), 3.54 (t, J=5.8 Hz, 2H), 3.02 (br t, J=5.6 Hz, 4H), 2.84-2.71 (m, 1H), 2.53-2.43 (m, 1H), 2.43-2.30 (m, 2H), 2.22-2.08 (m, 2H), 2.07-1.93 (m, 3H).

### Example 26 (General Procedure Z)

### (S)-1-(oxetan-2-ylmethyl)-2-((4-(3-(phenoxymethyl)-1H-pyrazol-1-yl)piperidin-1-yl)methyl)-1H-benzo[d]imidazole-6-carboxylic acid

### and

### (S)-1-(oxetan-2-ylmethyl)-2-((4-(5-(phenoxymethyl)-1H-pyrazol-1-yl)piperidin-1-yl)methyl)-1H-benzo[d]imidazole-6-carboxylic acid

The title compound was prepared according to Scheme 20. This General Procedure Z exemplifies Scheme 20 and provides particular synthetic details as applied to the title compound.

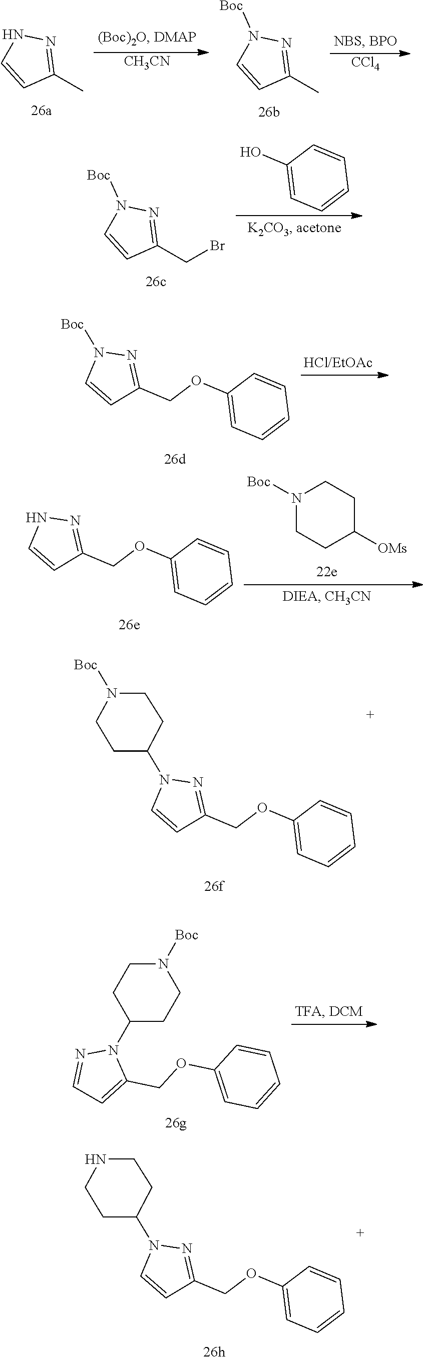

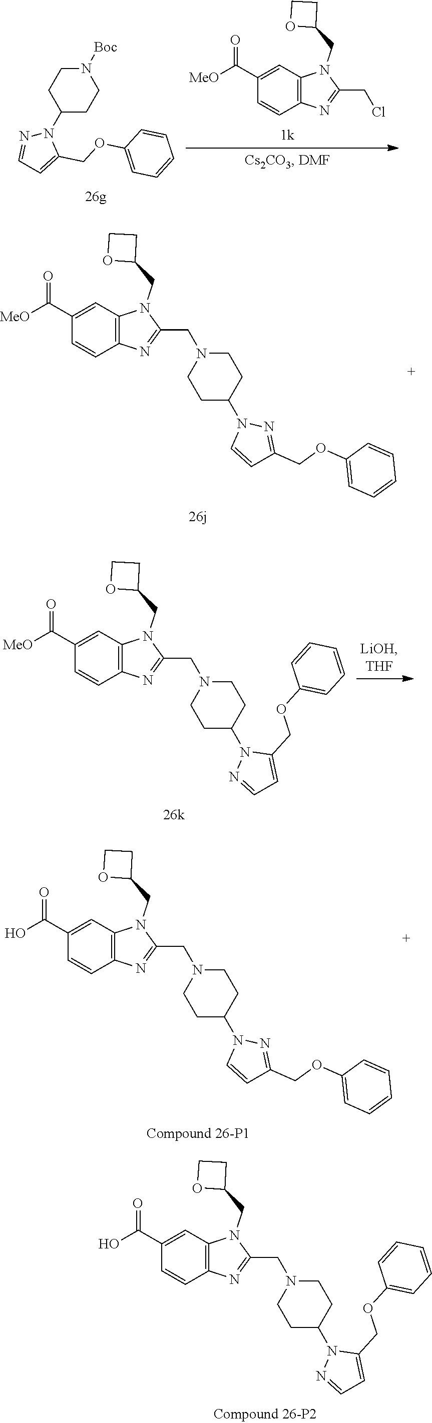 [SMILES: CC(C)(C)OC(=O)N1CCC(n2nccc2COc2ccccc2)CC1 ; COC(=O)c1ccc2nc(CCl)n(C[C@@H]3CCO3)c2c1 ; COC(=O)c1ccc2nc(CN3CCC(n4ccc(COc5ccccc5)n4)CC3)n(C[C@@H]3CCO3)c2c1 ; COC(=O)c1ccc2nc(CN3CCC(n4nccc4COc4ccccc4)CC3)n(C[C@@H]3CCO3)c2c1 ; O=C(O)c1ccc2nc(CN3CCC(n4ccc(COc5ccccc5)n4)CC3)n(C[C@@H]3CCO3)c2c1 ; O=C(O)c1ccc2nc(CN3CCC(n4nccc4COc4ccccc4)CC3)n(C[C@@H]3CCO3)c2c1]

### Tert-butyl 3-methyl-1H-pyrazole-1-carboxylate (26b)

To a solution of 3-methyl-1H-pyrazole (26a, 1g, 12.18 mmol, 1 eq) in CH3CN (10 mL) were added DMAP (1.49 g, 12.18 mmol, 1 eq) and (Boc)20 (3.19 g, 14.62 mmol, 3.36 mL, 1.2 eq) at 0° C. Then the mixture was stirred at 20° C. for 2.5 hours. LCMS showed 26a was consumed completely and one major peak with desired mass was detected. The reaction mixture was added H2O (10 mL), and then extracted with Ethyl acetate (10 mL*3). The combined organic layers were washed with brine (5 mL), dried over anhydrous Na2SO4, filtered and concentrated under reduced pressure to give 26b as yellow oil. The product was used directly for the next step without purification. 1H NMR (400 MHz, DMSO-d6) δ 7.68 (s, 1H), 5.89 (s, 1H), 2.05 (s, 3 H), 1.36 (s, 9H).

### Tert-butyl 3-(bromomethyl)-1H-pyrazole-1-carboxylate (26c)

To a solution of tert-butyl 3-methyl-1H-pyrazole-1-carboxylate (26b, 1.2 g, 6.59 mmol, 1 eq) in CCl4 (4 mL) were added BPO (159.52 mg, 658.55 umol, 0.1 eq) and NBS (1.17 g, 6.59 mmol, 1 eq) under N2. The mixture was stirred at 80° C. for 6 hours. LCMS showed 26b was consumed completely and one major peak with desired mass was detected. The reaction was diluted with H2O (30 mL) and then extracted with EtOAc (30 mL*3). The combined organic layers were washed with brine (30 mL), dried over anhydrous Na2SO4, filtered and concentrated under reduced pressure to give a residue. The residue was purified by column chromatography (SiO2, Petroleum ether:Ethyl acetate=5:1 to 3:1) to give 26c as white Oil.

### Tert-butyl 3-(phenoxymethyl)-1H-pyrazole-1-carboxylate (26d)

To a solution of tert-butyl 3-(bromomethyl)-1H-pyrazole-1-carboxylate (26c, 614 mg, 2.35 mmol, 1 eq) in acetone (10 mL) were added phenol (331.95 mg, 3.53 mmol, 310.23 uL, 1.5 eq) and K2CO3 (974.98 mg, 7.05 mmol, 3 eq) under N2. The mixture was stirred at 50° C. for 16 hours. LCMS showed 26c was consumed completely and one major peak with desired mass was detected. The mixture 26c was concentrated under reduced pressure. The residue was diluted with Ethyl acetate (30 mL). The organic layer was washed with water (10 mL), dried over anhydrous Na2SO4, filtered and concentrated under reduced pressure to give 26d was as white Oil. The product was used directly for the next step without purification. 1H NMR (400 MHz, CDCl3-d) δ 8.06 (d, J=2.8 Hz, 1H), 7.27-7.33 (m, 2H), 6.97-6.85 (m, 3H), 6.51 (d, J=2.6 Hz, 1H), 5.16 (s, 2H), 1.67 (s, 9H).

### 3-(phenoxymethyl)-1H-pyrazole (26e)

A solution of tert-butyl 3-(phenoxymethyl)-1H-pyrazole-1-carboxylate (26d, 170 mg, 619.73 umol, 1 eq) in HCl/EtOAc (4 M, 1 mL) was stirred at 20° C. for 2 hours. LCMS showed 26d was consumed completely and one major peak with desired mass was detected. The reaction mixture was concentrated under reduced pressure to give 26e as a white solid. The product was used directly in the next step without purification.

### Tert-butyl 4-(3-(phenoxymethyl)-1H-pyrazol-1-yl)piperidine-1-carboxylate (26f) and tert-butyl 4-(5-(phenoxymethyl)-1H-pyrazol-1-yl)piperidine-1-carboxylate (26g)

To a solution of 3-(phenoxymethyl)-1H-pyrazole (26e, 125 mg, 717.57 umol, 1 eq) in DMF (2 mL) were added tert-butyl 4-methylsulfonyloxypiperidine-1-carboxylate (601.37 mg, 2.15 mmol, 3 eq) and Cs2CO3 (584.50 mg, 1.79 mmol, 2.5 eq) under N2. The mixture was stirred at 80° C. for 16 hours. LCMS showed reactant was consumed completely and desired mass was detected. The residue was diluted with ethyl acetate (30 mL). The organic layer was washed with water (10 mL*3), dried over anhydrous Na2SO4, filtered and concentrated under reduced pressure to give a residue. The residue was purified by column chromatography (SiO2, Petroleum ether:Ethyl acetate=3:1) to give a mixture of 26f and 26g as colorless oil. 4-(3-(phenoxymethyl)-1H-pyrazol-1-yl)piperidine (26h) and 4-(5-(phenoxymethyl)-1H-pyrazol-1-yl)piperidine (26i).

The solution of tert-butyl 4-(3-(phenoxymethyl)-1H-pyrazol-1-yl)piperidine-1-carboxylate and tert-butyl 4-(5-(phenoxymethyl)-1H-pyrazol-1-yl)piperidine-1-carboxylate (26f and 26g 141 mg, 394.47 umol, 1 eq) in TFA (1 mL) and DCM (3 mL) was stirred at 20° C. for 2 hours under N2. LCMS showed 26f and 26g were consumed completely and desired mass was detected. The reaction mixture was concentrated under reduced pressure to give a mixture of 26h and 26i as colorless oil. The product was used directly in the next step without purification.

### (S)-methyl 1-(oxetan-2-ylmethyl)-2-((4-(3-(phenoxymethyl)-1H-pyrazol-1-yl)piperidin-1-yl)methyl)-1H-benzo[d]imidazole-6-carboxylate (26j) and (S)-methyl 1-(oxetan-2-ylmethyl)-2-((4-(5-(phenoxymethyl)-1H-pyrazol-1-yl)piperidin-1-yl)methyl)-1H-benzo[d]imidazole-6-carboxylate (26k)

To a solution of 4-(3-(phenoxymethyl)-1H-pyrazol-1-yl)piperidine and 4-(5-(phenoxymethyl)-1H-pyrazol-1-yl)piperidine (26h and 26i, 180 mg, 699.49 umol, 1 eq) in DMF (1 mL) were added methyl (S)-methyl 2-(chloromethyl)-1-(oxetan-2-ylmethyl)-1H-benzo[d]imidazole-6-carboxylate (1k, 206.16 mg, 699.49 umol, 1 eq) and K2CO3 (241.69 mg, 1.75 mmol, 2.5 eq) under N2. The mixture was stirred at 80° C. for 16 hours. LCMS showed 26h and 26i were consumed completely and desired mass was detected. The residue was diluted with Ethyl acetate (30 mL). The organic layer was washed with water (10 mL*3), dried over anhydrous Na2SO4, filtered and concentrated under reduced pressure. The residue was purified by column chromatography (SiO2, Dichloromethane:Methanol=10:1) to give a mixture of 26j and 26k as colorless oil.

### (S)-1-(oxetan-2-ylmethyl)-2-((4-(3-(phenoxymethyl)-1H-pyrazol-1-yl)piperidin-1-yl)methyl)-1H-benzo[d]imidazole-6-carboxylic acid (Compound 26-P1) and (S)-1-(oxetan-2-ylmethyl)-2-((4-(5-(phenoxymethyl)-1H-pyrazol-1-yl)piperidin-1-yl)methyl)-1H-benzo[d]imidazole-6-carboxylic acid (Compound 26-P2)

To a solution of (S)-methyl 1-(oxetan-2-ylmethyl)-2-((4-(3-(phenoxymethyl)-1H-pyrazol-1-yl)piperidin-1-yl)methyl)-1H-benzo[d]imidazole-6-carboxylate and (S)-methyl 1-(oxetan-2-ylmethyl)-2-((4-(5-(phenoxymethyl)-1H-pyrazol-1-yl)piperidin-1-yl)methyl)-1H-benzo[d]imidazole-6-carboxylate (26j and 26k, 145 mg, 281.22 umol, 1 eq) in THF (0.7 mL) and H2O (0.3 mL) was added the solution of LiOH·H2O (35.40 mg, 843.67 umol, 3 eq) under N2. The mixture was stirred at 20° C. for 24 hours. TLC (Dichloromethane:Methanol=10:1) indicated 26j and 26k were consumed completely and one new spot was formed. The mixture was adjusted to pH=6 with citric acid (1 M). Then the mixture was extracted with Ethyl acetate (10 mL*2). The combined organic layers were dried over Na2SO4, filtered and concentrated in vacuo to give a mixture of two isomers. The two isomers were separated by Chiral SFC (Instrument: Waters SFC80 preparative SFC; Column: Chiralpak AD, 250*30 mm i.d. 10 um; Mobile phase: A for CO2 and B for EtOH (0.1% NH3H2O); Gradient: B %=50% isocratic elution mode; Flow rate: 70 g/min; Column temperature: 40oC System back pressure: 100 bar) to afford the Compound 26-P1 as a white solid. MS mass calculated for [M+1]+(C28H31N5O4) requires m/z 501.2, LCMS found m/z 502.3. 1H NMR (400 MHz, MeOD-d4) δ 8.34 (s, 1H), 7.97 (d, J=8.4 Hz, 1H), 7.68-7.65 (m, 2H), 7.65 (m, 2H), 7.25 (t, J=7.6 Hz, 2H), 6.98-6.90 (m, 3H), 6.34 (s, 1H), 5.28-5.26 (m, 1H), 5.02 (s, 2H), 4.89-4.72 (m, 2H), 4.66-4.64 (m, 1H), 4.48-4.46 (m, 1H), 4.08-4.06 (m, 1H), 4.00-3.98 (m, 1H), 3.93-3.90 (m, 1H), 3.10-3.07 (d, J=11.6 Hz, 1H), 2.98 (d, J=11.2 Hz, 1H), 2.91-2.75 (m, 1H), 2.52-2.50 (m, 1H), 2.40-2.39 (m, 2H), 2.11-2.03 (m, 4H).

Compound 26-P2 was obtained as a white solid. MS mass calculated for [M+1]+(C28H31N5O4) requires m/z 501.2, LCMS found m/z 502.3. 1H NMR (400 MHz, MeOD-d4) δ 8.34 (s, 1H) 7.96 (d, J=8 Hz, 1H), 7.65 (d, J=8.4, 1H), 7.47 (s, 1H), 7.29 (t, J=7.6 Hz, 2H), 7.01-6.59 (m, 3H), 6.36 (s, 1H), 5.28-5.27 (m, 1H), 5.17 (s, 2H), 4.89-4.87 (m, 1H), 4.75-4.65 (m, 2H), 4.49-4.47 (m, 1H), 4.31-4.29 (m, 1H), 4.05-4.02 (d, J=13.6 Hz, 1H), 3.92-3.88 (d, J=14 Hz, 1H), 3.09-3.06 (m, 1H), 2.95-2.93 (m, 2H), 2.35-2.32 (m, 1H), 2.28-2.22 (m, 4H), 1.98-1.95 (m, 2H).

### Example 27 (General Procedure AA)

### (S)-2-((4-(1-benzyl-1H-pyrazolo[3,4-b]pyridin-6-yl)piperazin-1-yl)methyl)-1-(oxetan-2-ylmethyl)-1H-benzo[d]imidazole-6-carboxylic acid

The title compound was prepared according to Scheme 21. This General Procedure AA exemplifies Scheme 21 and provides particular synthetic details as applied to the title compound.

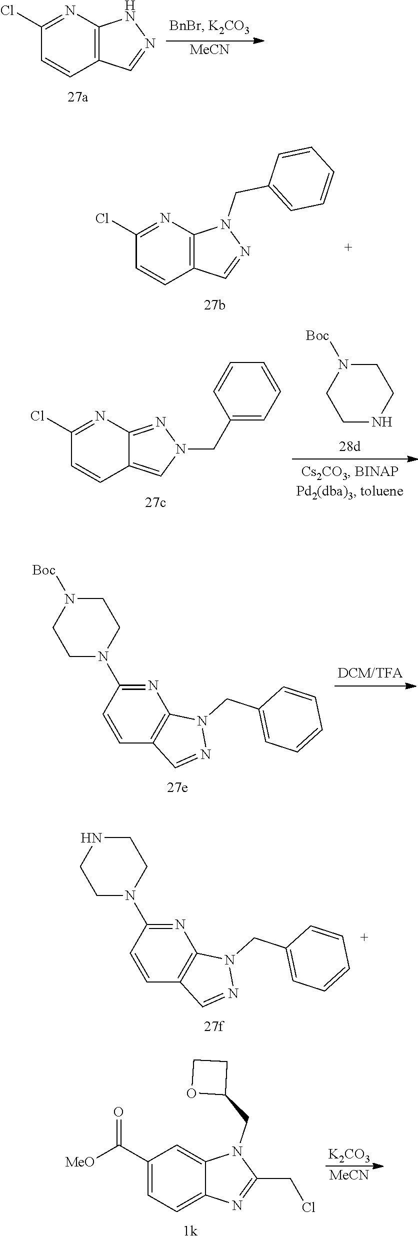

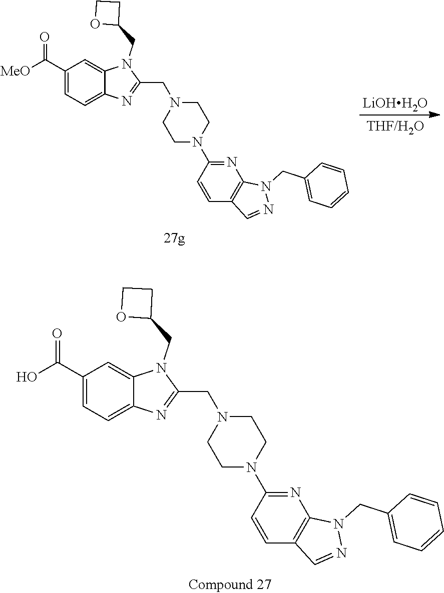 [SMILES: COC(=O)c1ccc2nc(CN3CCN(c4ccc5cnn(Cc6ccccc6)c5n4)CC3)n(C[C@@H]3CCO3)c2c1 ; "LiOH·H2O" ; O=C(O)c1ccc2nc(CN3CCN(c4ccc5cnn(Cc6ccccc6)c5n4)CC3)n(C[C@@H]3CCO3)c2c1]

### 1-benzyl-6-chloro-1H-pyrazolo[3,4-b]pyridine (27b) & 2-benzyl-6-chloro-2H-pyrazolo[3,4-b]pyridine (27c)

To a solution of 6-chloro-1H-pyrazolo[3,4-b]pyridine (27a, 3 g, 19.54 mmol, 1 eq), bromomethylbenzene (5.01 g, 29.30 mmol, 3.48 mL, 1.5 eq) in MeCN (50 mL) was added K2CO3 (8.10 g, 58.61 mmol, 3 eq). The mixture was stirred at 60° C. for 1 hour. LCMS showed desired mass was detected. The reaction mixture was concentrated under reduced pressure to remove solvent. The residue was purified by prep-TLC (SiO2, Petroleum ether:Ethyl acetate=3:1) to give 27b as a white solid. 1H NMR (400 MHz, CDCl3-d) δ 8.11-7.92 (m, 2H), 7.41-7.28 (m, 5H), 7.16 (d, J=8.4 Hz, 1H), 5.69 (s, 2H). And 27c was obtained as a white solid. 1H NMR (400 MHz, CDCl3-d) δ 7.94-7.92 (d, J=8.0 Hz, 2H), 7.85 (s, 1H), 7.41-7.37 (m, 5H), 7.06-7.03 (d, J=8.0 Hz, 2H), 5.60 (s, 2H).

### Tert-butyl 4-(1-benzyl-1H-pyrazolo[3,4-b]pyridin-6-yl)piperazine-1-carboxylate (27e)

A mixture of 1-benzyl-6-chloro-1H-pyrazolo[3,4-b]pyridine (27b, 600 mg, 2.46 mmol, 1 eq), tert-butyl piperazine-1-carboxylate (27d, 917.15 mg, 4.92 mmol, 2 eq), Cs2CO3 (1.60 g, 4.92 mmol, 2 eq), Pd2(dba)3 (112.73 mg, 123.11 umol, 0.05 eq) and BINAP (153.31 mg, 246.21 umol, 0.1 eq) in toluene (10 mL) was degassed and purged with N2 3 times, and then the mixture was stirred at 120° C. for 16 hours under N2 atmosphere. TLC (Petroleum ether:Ethyl acetate=3:1) showed a new spot was generated. The reaction mixture was filtered and concentrated under reduced pressure to give a residue. The residue was purified by column chromatography (SiO2, Petroleum ether:Ethyl acetate=50:1 to 1:1) to give 27e as a white solid. 1H NMR (400 MHz, CDCl3-d) δ 7.84-7.72 (m, 2H), 7.32 (br d, J=14.2 Hz, 4H), 7.26-7.21 (m, 1H), 6.60 (d, J=8.8 Hz, 1H), 5.55 (s, 2H), 3.76-3.63 (m, 4H), 3.62-3.50 (m, 4H), 1.50 (s, 9H).

### 1-benzyl-6-(piperazin-1-yl)-1H-pyrazolo[3,4-b]pyridine (27f)

To a solution of tert-butyl 4-(1-benzyl-1H-pyrazolo[3,4-b]pyridin-6-yl)piperazine-1-carboxylate (27e, 125 mg, 317.68 umol, 1 eq) in DCM (2 mL) was added TFA (1.93 g, 16.88 mmol, 1.25 mL, 53.14 eq). The mixture was stirred at 15° C. for 2 hours. TLC (Petroleum ether:Ethyl acetate=3:1, Rf=0) showed a new spot was generated. The reaction mixture was concentrated under reduced pressure to give 27f as a white solid. 1H NMR (400 MHz, DMSO-d6) δ 8.82 (br s, 2H), 7.99 (d, J=8.8 Hz, 1H), 7.88 (s, 1H), 7.36-7.21 (m, 3H), 6.88 (d, J=8.8 Hz, 1H), 5.50 (s, 2H), 3.95-3.78 (m, 4H), 3.22 (br s, 4H).

### (S)-methyl 2-((4-(1-benzyl-1H-pyrazolo[3,4-b]pyridin-6-yl)piperazin-1-yl)methyl)-1-(oxetan-2-ylmethyl)-1H-benzo[d]imidazole-6-carboxylate (27g)

To a solution of 1-benzyl-6-(piperazin-1-yl)-1H-pyrazolo[3,4-b]pyridine (27f, 129 mg, 316.65 umol, 1 eq, TFA) and (S)-methyl 2-(chloromethyl)-1-(oxetan-2-ylmethyl)-1H-benzo[d]imidazole-6-carboxylate (1k, 102.66 mg, 348.32 umol, 1.1 eq) in ACN (2 mL) was added K2CO3 (218.82 mg, 1.58 mmol, 5 eq). The mixture was stirred at 50° C. for 16 hours. LCMS showed 27f was consumed and desired mass was detected. The reaction mixture was concentrated under reduced pressure. The residue was purified by prep-TLC (SiO2, Petroleum ether:Ethyl acetate=1:1) to give 27g as a white solid. 1H NMR (400 MHz, CDCl3-d) δ 8.18 (d, J=1.2 Hz, 1H), 7.99 (dd, J=1.6, 8.5 Hz, 1H), 7.83-7.71 (m, 3H), 7.36-7.28 (m, 4H), 7.26-7.19 (m, 1H), 6.59 (d, J=9.2 Hz, 1H), 5.53 (s, 2H), 5.32-5.19 (m, 1H), 4.83-4.55 (m, 3H), 4.40 (td, J=6.0, 9.2 Hz, 1H), 4.03 (d, J=3.6 Hz, 2H), 3.97 (s, 3H), 3.74-3.65 (m, 4H), 2.81-2.73 (m, 1H), 2.72-2.65 (m, 4H), 2.53-2.40 (m, 1H).

### (S)-2-((4-(1-benzyl-1H-pyrazolo[3,4-b]pyridin-6-yl)piperazin-1-yl)methyl)-1-(oxetan-2-ylmethyl)-1H-benzo[d]imidazole-6-carboxylic acid (27)

To a solution of (S)-methyl 2-((4-(1-benzyl-1H-pyrazolo[3,4-b]pyridin-6-yl)piperazin-1-yl)methyl)-1-(oxetan-2-ylmethyl)-1H-benzo[d]imidazole-6-carboxylate (27g, 75 mg, 135.96 umol, 1 eq) in THF (1.4 mL) and H2O (0.6 mL) was added LiOH·H2O (17.12 mg, 407.88 umol, 3 eq). The mixture was stirred at 15° C. for 16 hours. LCMS showed 27g was consumed and desired mass was detected. Then citric acid solution (1 M) was added in the mixture until pH=6. The mixture was filtered, the filter cake was washed with water for 3 times and dried over in vacuo to give Compound 27 as a white solid. MS mass calculated for [M+1]+(C30H31N7O3) requires m/z 538.2, LCMS found m/z 538.3; 1H NMR (400 MHz, MeOD-d4) δ 8.36 (s, 1H), 8.01-7.95 (m, 1H), 7.87 (d, J=9.0 Hz, 1H), 7.78 (s, 1H), 7.69 (d, J=8.4 Hz, 1H), 7.30-7.18 (m, 5H), 6.79 (d, J=9.0 Hz, 1H), 5.50 (s, 2H), 5.28 (br d, J=5.6 Hz, 1H), 4.92 (br d, J=7.6 Hz, 1H), 4.79-4.71 (m, 1H), 4.69-4.60 (m, 1H), 4.52-4.42 (m, 1H), 4.12-3.91 (m, 2H), 3.75 (br s, 4H), 2.84-2.77 (m, 1H), 2.67 (br d, J=5.6 Hz, 4H), 2.59-2.47 (m, 1H).

### Example 28 (General Procedure BB)

### (S)-2-((4-(3-benzyl-3H-imidazo[4,5-b]pyridin-5-yl)piperazin-1-yl)methyl)-1-(oxetan-2-ylmethyl)-1H-benzo[d]imidazole-6-carboxylic acid

The title compound was prepared according to Scheme 21. This General Procedure BB exemplifies Scheme 21 and provides particular synthetic details as applied to the title compound.

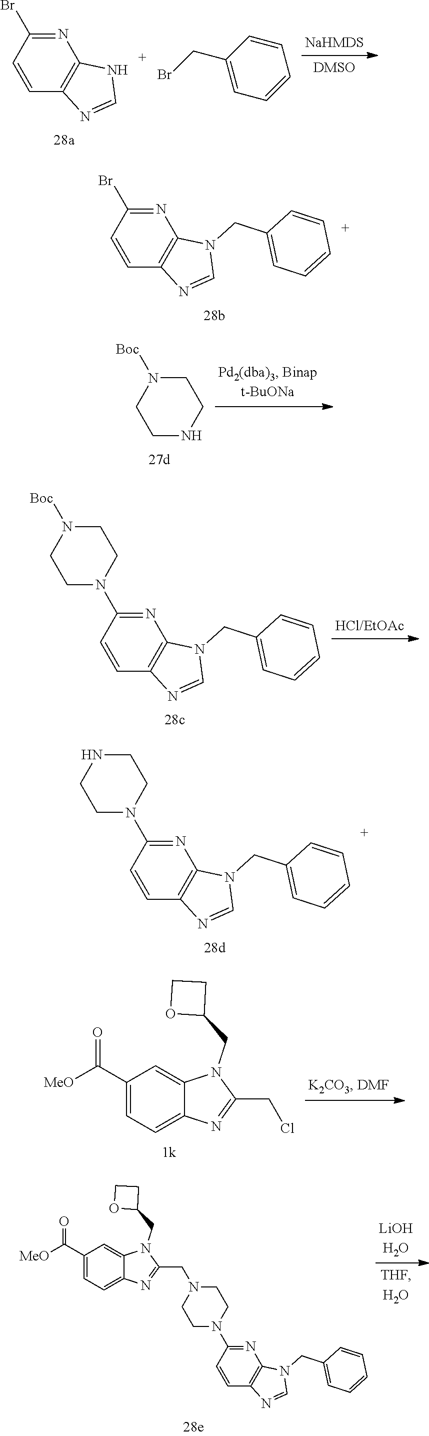

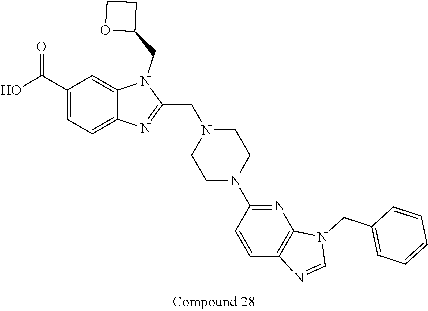 [SMILES: O=C(O)c1ccc2nc(CN3CCN(c4ccc5ncn(Cc6ccccc6)c5n4)CC3)n(C[C@@H]3CCO3)c2c1]

### 3-benzyl-5-bromo-3H-imidazo[4,5-b]pyridine (28b)

To a solution of 5-bromo-3H-imidazo[4,5-b]pyridine (28a, 200 mg, 1.01 mmol, 1 eq) and (bromomethyl)benzene (518.23 mg, 3.03 mmol, 359.88 uL, 3 eq) in DMSO (5 mL) was added NaHMDS (1 M, 1.51 mL, 1.5 eq). The mixture was stirred at 20° C. for 1.5 hours. TLC (Ethyl acetate:Petroleum ether=3:1) indicated 28a was consumed completely and two new spots were formed. The reaction mixture was diluted with water (15 mL) and extracted with Ethyl acetate (10 mL*3). The combined organic layers were washed with brine (10 mL), dried over anhydrous Na2SO4, filtered and concentrated under reduced pressure. The residue was purified by prep-TLC (SiO2, Ethyl acetate:Petroleum ether:TEA=3:1:0.05) to give 28b as a light yellow solid.

### Tert-butyl 4-(3-benzyl-3H-imidazo[4,5-b]pyridin-5-yl)piperazine-1-carboxylate (28c)

A mixture of 3-benzyl-5-bromo-3H-imidazo[4,5-b]pyridine (28b, 66 mg, 229.05 umol, 1 eq), tert-butyl piperazine-1-carboxylate (27d, 46.93 mg, 251.96 umol, 1.1 eq), BINAP (21.39 mg, 34.36 umol, 0.15 eq) and t-BuONa (33.02 mg, 343.58 umol, 1.5 eq), Pd2(dba)3 (10.49 mg, 11.45 umol, 0.05 eq) in toluene. (3 mL) was degassed and purged with N2 3 times, and then the mixture was stirred at 110° C. for 12 hours under N2 atmosphere. LCMS showed 28b was consumed and desired mass was detected. The reaction mixture was diluted with water (10 mL) and extracted with Ethyl acetate (10 mL*3). The combined organic layers were washed with brine (10 mL), dried over anhydrous Na2SO4, filtered and concentrated under reduced pressure. The residue was purified by prep-TLC (SiO2, Ethyl acetate:Petroleum ether=3:1) to give 28c as a light yellow oil.

### 3-benzyl-5-(piperazin-1-yl)-3H-imidazo[4,5-b]pyridine (28d)

To a solution of tert-butyl 4-(3-benzyl-3H-imidazo[4,5-b]pyridin-5-yl)piperazine-1-carboxylate (28c, 200 mg, 508.28 umol, 1 eq) in HCl/EtOAc (10 mL). The mixture was stirred at 20° C. for 2 hours. LCMS showed 29c was consumed completely and one major peak with desired mass was detected. The reaction mixture was filtered, and the filter liquor was concentrated under reduced pressure to give 28d as a yellow solid. The residue was used directly in the next step without purification.

### (S)-methyl 2-((4-(3-benzyl-3H-imidazo[4,5-b]pyridin-5-yl)piperazin-1-yl)methyl)-1-(oxetan-2-ylmethyl)-1H-benzo[d]imidazole-6-carboxylate (28e)

To a solution of 3-benzyl-5-(piperazin-1-yl)-3H-imidazo[4,5-b]pyridine (28d, 325 mg, 498.52 umol, 1 eq) in DMF (2 mL) was added K2CO3 (339.21 mg, 2.45 mmol, 5 eq) and (S)-methyl 2-(chloromethyl)-1-(oxetan-2-ylmethyl)-1H-benzo[d]imidazole-6-carboxylate (1k, 100 mg, 339.29 umol, 0.7 eq) under N2. The mixture was stirred at 50° C. for 6 hours. LCMS showed 28d was consumed completely and one major peak with desired mass was formed. The suspension was filtered through a pad of Celite and the pad cake was washed with Ethyl acetate (5 mL*3). The residue was extracted with Ethyl acetate (5 mL*3) and H2O (5 mL). The combined organic layers were washed with brine (5 mL), dried over anhydrous Na2SO4, filtered and concentrated under reduced pressure. The residue was purified by column chromatography (SiO2, Ethyl acetate:Methanol=5:1) to give 28e as a yellow solid, 1H NMR (400 MHz, CDCl3-d) 8.20-8.15 (m, 1H), 8.04-7.96 (m, 1H), 7.91-7.84 (m, 1H), 7.83-7.74 (m, 2H), 7.38-7.28 (m, 5H), 6.67 (d, J=8.8 Hz, 1H), 5.35-5.30 (m, 1H), 5.32 (d, J=6.4 Hz, 1H), 5.29-5.21 (m, 1H), 4.82-4.54 (m, 3H), 4.46-4.31 (m, 1H), 3.96 (s, 3H), 3.55-3.69 (m, 1H), 3.72-3.54 (m, 3H), 3.50 (s, 1H), 2.82-2.72 (m, 1H), 2.70 (br t, J=4.8 Hz, 2H), 2.82-2.61 (m, 1H), 2.53-2.39 (m, 1H).

### (S)-2-((4-(3-benzyl-3H-imidazo[4,5-b]pyridin-5-yl)piperazin-1-yl)methyl)-1-(oxetan-2-ylmethyl)-1H-benzo[d]imidazole-6-carboxylic acid (Compound 28)

To a solution of (S)-methyl 2-((4-(3-benzyl-3H-imidazo[4,5-b]pyridin-5-yl)piperazin-1-yl)methyl)-1-(oxetan-2-ylmethyl)-1H-benzo[d]imidazole-6-carboxylate (28e, 110 mg, 199.41 umol, 1 eq) in THF (1.4 mL) and was added the solution of LiOH·H2O (20.92 mg, 498.52 umol, 2.5 eq) in H2O (0.6 mL) under N2. The mixture was stirred at 20° C. for 16 hours. LiOH·H2O (4.18 mg, 99.70 umol, 0.5 eq) was added in the mixture, and the reaction mixture was stirred at 25° C. for another 24 hours. LCMS showed 28e was consumed completely and one major peak with desired mass was formed. The mixture was adjusted to pH=6 with Citric acid (1 M, aq). Then the mixture was concentrated under reduced pressure to remove THF. The crude product was purified by reversed-phase HPLC (column: Phenomenex Gemini-NX C18 75*30 mm*3 um; mobile phase: [water (10 mM NH4HCO3)-ACN]; B %: 5%-30%, 10 min) to give Compound 28 as a white solid. MS mass calculated for [M+H]+ (C30H31N7O3) requires m/z 538.3, LCMS found m/z 538.3. 1H NMR (400 MHz, DMSO-d6) δ 8.27 (s, 1H), 8.20 (s, 1H), 7.81 (d, J=8.6 Hz, 2H), 7.65 (d, J=8.4 Hz, 1H), 7.41-7.35 (m, 2H), 7.32 (t, J=7.2 Hz, 2H), 7.28-7.23 (m, 1H), 6.77 (d, J=8.6 Hz, 1H), 5.32 (s, 2H), 5.11 (br d, J=7.2 Hz, 1H), 4.80 (dd, J=15.0, 7.6 Hz, 1H), 4.71-4.62 (m, 1H), 4.53-4.44 (m, 1H), 4.42-4.33 (m, 1H), 3.99 (d, J=13.2 Hz, 1H), 3.81 (d, J=13.4 Hz, 1H), 3.53 (br s, 4H), 2.77-2.54 (m, 5H), 2.45 (br d, J=7.8 Hz, 1H).

### Example 29 (General Procedure CC)

### (S)-2-((4-(1-(4-cyano-2-fluorobenzyl)-1H-pyrrolo[2,3-b]pyridin-6-yl)piperazin-1-yl)methyl)-1-(oxetan-2-ylmethyl)-1H-benzo[d]imidazole-6-carboxylic acid

The title compound was prepared according to Scheme 21. This General Procedure CC exemplifies Scheme 21 and provides particular synthetic details as applied to the title compound.

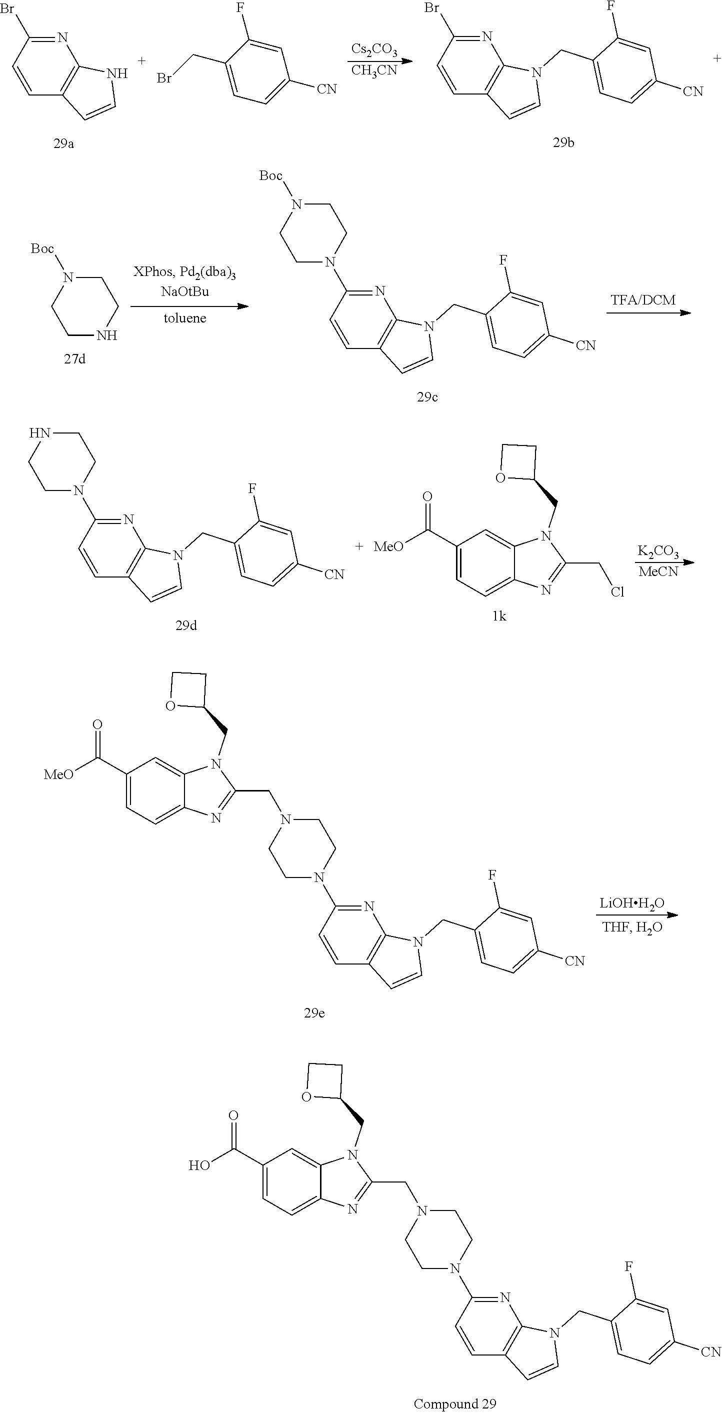

### 4-((6-bromo-1H-pyrrolo[2,3-b]pyridin-1-yl)methyl)-3-fluorobenzonitrile (29b)

To a solution of 6-bromo-1H-pyrrolo[2,3-b]pyridine (29a, 0.3 g, 1.52 mmol, 1 eq) in CH3CN (2 mL) was added 4-(bromomethyl)-3-fluorobenzonitrile (651.78 mg, 3.05 mmol, 2 eq) and Cs2CO3 (992.18 mg, 3.05 mmol, 2 eq) under N2 at 25° C. The mixture was stirred at 50° C. for 16 hours. LCMS showed the reactant was consumed and desired product was detected. The residue was poured into water (5 mL). The aqueous layers were extracted with ethyl acetate (5 mL*2). The combined organic layers were washed with brine (5 mL*2), dried with anhydrous Na2SO4, filtered and concentrated in vacuo. The crude product was purified by column chromatography (SiO2, petroleum ether:ethyl acetatee=20:1 to 1:1) to give 29b as a white solid. 1HNMR (400 MHz, CDCl3-d) δ 7.81 (d, J=8.2 Hz, 1H), 7.40 (t, J=9.6 Hz, 2 H), 7.29 (s, 1H), 7.15-7.23 (m, 2H), 6.55 (d, J=3.6 Hz, 1H), 5.58 (s, 2H).

### Tert-butyl 4-(1-(4-cyano-2-fluorobenzyl)-1H-pyrrolo[2,3-b]pyridin-6-yl)piperazine-1-carboxylate (29c)

To a solution of 4-((6-bromo-1H-pyrrolo[2,3-b]pyridin-1-yl)methyl)-3-fluorobenzonitrile (29b, 450 mg, 1.36 mmol, 1 eq) and tert-butyl piperazine-1-carboxylate (27d 761.58 mg, 4.09 mmol, 3 eq) in toluene (10 mL) was added XPhos (129.95 mg, 272.60 umol, 0.2 eq), Pd2(dba)3 (124.81 mg, 136.30 umol, 0.1 eq) and NaOtBu (196.48 mg, 2.04 mmol, 1.5 eq) under N2 at 25° C. The suspension was degassed under vacuum and purged with N2 several times. The mixture was stirred at 100° C. for 16 hours under N2. LCMS showed that 29b was consumed and the desired product mass was detected. The mixture was poured into water (10 mL), and extracted with ethyl acetate (10 mL*2). The combined organic layers were washed with brine (5 mL*2), dried with anhydrous Na2SO4, filtered and concentrated in vacuo. The crude product was purified by silica gel chromatography (petroleum ether:ethyl acetate=50:1 to 1:1) to give 29c as a yellow solid. 1HNMR (400 MHz, CDCl3-d) δ 7.76 (d, J=8.6 Hz, 1H), 7.38 (dd, J=9.2, 1.4 Hz, 1H), 7.33 (dd, J=7.8, 1.2 Hz, 1H), 7.13 (t, J=7.6 Hz, 1H), 6.95 (d, J=3.6 Hz, 1H), 6.59 (d, J=8.8 Hz, 1H), 6.38 (d, J=3.4 Hz, 1H), 5.46 (s, 2H), 3.49-3.61 (m, 8H), 1.50 (s, 9H).

### 3-fluoro-4-((6-(piperazin-1-yl)-1H-pyrrolo[2,3-b]pyridin-1-yl)methyl)benzonitrile (29d)

To a solution of tert-butyl 4-(1-(4-cyano-2-fluorobenzyl)-1H-pyrrolo[2,3-b]pyridin-6-yl) piperazine-1-carboxylate (29c, 350 mg, 803.69 umol, 1 eq) in dichloromethane (4 mL) was added TFA (2 mL) at 25° C. The mixture was stirred at 25° C. for 2 hours. TLC (petroleum ether:ethyl acetate=3:1) showed that 29c was consumed. The mixture was concentrated in vacuo to give 29d as yellow oil. The product was used directly in next step.

### (S)-methyl 2-((4-(1-(4-cyano-2-fluorobenzyl)-1H-pyrrolo[2,3-b]pyridin-6-yl)piperazin-1-yl)methyl)-1-(oxetan-2-ylmethyl)-1H-benzo[d]imidazole-6-carboxylate (29e)

To a solution of 3-fluoro-4-((6-(piperazin-1-yl)-1H-pyrrolo[2,3-b]pyridin-1-yl)methyl)benzonitrile (29d, 350 mg, 778.82 umol, 1 eq, TFA) in CH3CN (5 mL) was added K2CO3 (322.91 mg, 2.34 mmol, 3 eq). The mixture was stirred at 25° C. for 0.5 hour. Then (S)-methyl 2-(chloromethyl)-1-(oxetan-2-ylmethyl)-1H-benzo[d]imidazole-6-carboxylate (1k, 252.50 mg, 856.70 umol, 1.1 eq) was added to the mixture, and the mixture was stirred at 80° C. for 2.5 hours. TLC (petroleum ether:ethyl acetatee=0:1) showed that 1k was consumed and one major spot was formed. The residue was poured into water (5 mL). The aqueous layers were extracted with ethyl acetate (5 mL*2). The combined organic layers were washed with brine (5 mL*1), dried with anhydrous Na2SO4, filtered and concentrated in vacuo. The crude product was purified by silica gel chromatography (petroleum ether:ethyl acetatee=10:1 to 0:1) to give 29e as a white solid. 1H NMR (400 MHz, CDCl3-d) δ 8.18 (d, J=1.2 Hz, 1H), 7.99 (dd, J=8.4, 1.6 Hz, 1H), 7.75 (dd, J=13.6, 8.6 Hz, 2H), 7.36 (dd, J=9.2, 1.6 Hz, 1H), 7.31 (dd, J=7.8, 1.2 Hz, 1H), 7.13 (t, J=7.6 Hz, 1H), 6.95 (d, J=3.6 Hz, 1H), 6.58 (d, J=8.6 Hz, 1H), 6.37 (d, J=3.6 Hz, 1H), 5.43 (s, 2H), 5.20-5.29 (m, 1H), 4.70-4.80 (m, 2H), 4.59-4.69 (m, 1H), 4.40 (dt, J=9.0, 6.0 Hz, 1H), 4.00-4.08 (m, 2 H), 3.96 (s, 3H), 3.56 (s, 4H), 2.65-2.80 (m, 4H), 2.40-2.54 (m, 1H).

### (S)-2-((4-(1-(4-cyano-2-fluorobenzyl)-1H-pyrrolo[2,3-b]pyridin-6-yl)piperazin-1-yl)methyl)-1-(oxetan-2-ylmethyl)-1H-benzo[d]imidazole-6-carboxylic acid (Compound 29)

To a solution of (S)-methyl 2-((4-(1-(4-cyano-2-fluorobenzyl)-1H-pyrrolo[2,3-b]pyridin-6-yl)piperazin-1-yl)methyl)-1-(oxetan-2-ylmethyl)-1H-benzo[d]imidazole-6-carboxylate (29e, 0.15 g, 252.67 umol, 1 eq) in THF (2.1 mL) was added the solution of LiOH·H2O (10.60 mg, 252.67 umol, 1 eq) in H2O (0.9 mL) at 25° C. The mixture was stirred at 25° C. for 16 hours. LCMS showed the 29e was consumed, and desired product mass was detected in the major peak. The mixture was added critic acid until pH=7. The solution was concentrated in vacuo. The residue was diluted in MeOH (5 mL), and filtered. The filtrate was concentrated in vacuo. The residue was purified by prep-HPLC (column: Waters Xbridge Prep OBD C18 150*40 mm*10 um; mobile phase: [water (10 mM NH4HCO3)-ACN]; B %: 15%-50%, 8 min) to give Compound 29 as a white solid. MS mass calculated for [M+H]+ (C32H30FN703) requires m/z 580.2 LCMS found m/z 580.2; 1H NMR (400 MHz, CDCl3-d) δ 8.23 (s, 1H) 8.06 (dd, J=8.6, 1.4 Hz, 1H), 7.82 (d, J=8.6 Hz, 1H), 7.73 (d, J=8.6 Hz, 1H), 7.35 (dd, J=9.2, 1.2 Hz, 1H), 7.28-7.32 (m, 1H), 7.12 (t, J=7.6 Hz, 1H), 6.94 (d, J=3.6 Hz, 1H), 6.57 (d, J=8.8 Hz, 1H), 6.36 (d, J=3.6 Hz, 1H), 5.43 (s, 2H), 5.20-5.29 (m, 1H), 4.70-4.80 (m, 2H), 4.59-4.69 (m, 1H), 4.41 (dt, J=9.2, 6.0 Hz, 1H), 3.99-4.09 (m, 2H), 3.55 (s, 4H), 2.74-2.81 (m, 1H), 2.70 (t, J=4.8 Hz, 4H), 2.47 (ddd, J=16.2, 11.2, 7.4 Hz, 1H).

### Example 30 (General Procedure DD)

### (S)-2-((4-(1-(4-cyano-2-fluorobenzyl)-1H-pyrazolo[3,4-b]pyridin-6-yl)piperazin-1-yl)methyl)-1-(oxetan-2-ylmethyl)-1H-benzo[d]imidazole-6-carboxylic acid

The title compound was prepared according to Scheme 21. This General Procedure DD exemplifies Scheme 21 and provides particular synthetic details as applied to the title compound.

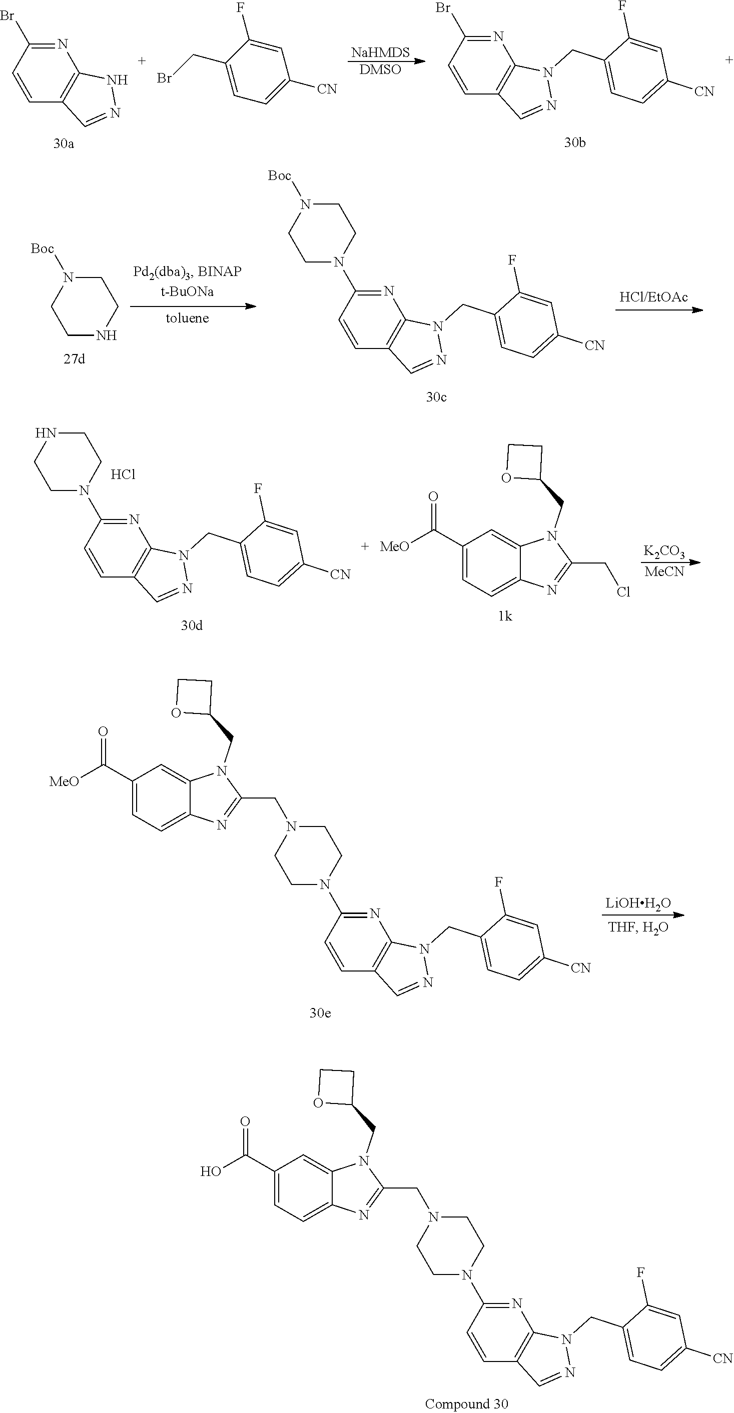

### 4-((6-bromo-1H-pyrazolo[3,4-b]pyridin-1-yl)methyl)-3-fluorobenzonitrile (30b)

To a solution of 6-bromo-1H-pyrazolo[3,4-b]pyridine (30a, 845 mg, 4.27 mmol, 1 eq) and NaHMDS (1 M, 6.40 mL, 1.5 eq) in DMSO (20 mL) was added 4-(bromomethyl)-3-fluorobenzonitrile (2.74 g, 12.80 mmol, 3 eq) at 20° C. Then the solution was stirred at 20° C. for 2 hours. TLC (petroleum ether:ethyl acetate=3:1) showed 30a was consumed and two new spots were formed. The mixture was quenched with saturated aqueous NH4Cl (60 mL) and extracted with ethyl acetate (20 mL*3). The combined ethyl acetate was washed with brine (20 mL), dried over anhydrous Na2SO4, filtered and concentrated in vacuo. The residue was purified by column chromatography (SiO2, petroleum ether:ethyl acetate=80:1 to 20:1) to give 30b as a white solid. 1H NMR (400 MHz, MeOD-d4) δ 8.20-8.08 (m, 2H), 7.59 (d, J=10.8 Hz, 1H), 7.49 (d, J=8.0 Hz, 1H), 7.41 (d, J=8.4 Hz, 1H), 7.25 (t, J=7.6 Hz, 1H), 5.80 (s, 2H).

### Tert-butyl 4-(1-(4-cyano-2-fluorobenzyl)-1H-pyrazolo[3,4-b]pyridin-6-yl)piperazine-1-carboxylate (30c)

Pd2(dba)3 (40.10 mg, 43.79 umol, 0.05 eq), BINAP (54.53 mg, 87.58 umol, 0.1 eq) and t-BuONa (252.49 mg, 2.63 mmol, 3 eq) was added to the solution of 4-((6-bromo-1H-pyrazolo[3,4-b]pyridin-1-yl)methyl)-3-fluorobenzonitrile (30b, 290 mg, 875.76 umol, 1 eq) and tert-butyl piperazine-1-carboxylate (27d, 326.22 mg, 1.75 mmol, 2 eq) in toluene (15 mL) at 20° C. under N2. Then the solution was stirred at 100° C. for 16 hours under N2. TLC (petroleum ether:ethyl acetate=3:1,) showed 30b was consumed and one major new spot was formed. The mixture was concentrated in vacuo. The residue was purified by column chromatography (SiO2, petroleum ether:ethyl acetate=80:1 to 20:1) to give 30c as a yellow solid. 1H NMR (400 MHz, MeOD-d4) δ 7.90 (d, J=9.0 Hz, 1H), 7.84 (s, 1H), 7.59 (dd, J=1.2, 9.6 Hz, 1H), 7.47 (d, J=7.8 Hz, 1H), 7.21 (t, J=7.6 Hz, 1H), 6.81 (d, J=9.0 Hz, 1H), 5.66 (s, 2H), 3.73-3.67 (m, 4H), 3.56-3.50 (m, 4H), 1.49 (s, 9H).

### 3-fluoro-4-((6-(piperazin-1-yl)-1H-pyrazolo[3,4-b]pyridin-1-yl)methyl)benzonitrile (30d)

The solution of tert-butyl 4-(1-(4-cyano-2-fluorobenzyl)-1H-pyrazolo[3,4-b]pyridin-6-yl)piperazine-1-carboxylate (30c, 123 mg, 260.07 umol, 1 eq, HCl) in HCl/EtOAc (1 mL) was stirred at 20° C. for 0.5 hours. LCMS showed 30d was consumed, and desired mass was detected. The mixture was concentrated in vacuo at 20° C. to give 30d as a yellow solid. The product was used directly in next step without any further purification.

### (S)-methyl 2-((4-(1-(4-cyano-2-fluorobenzyl)-1H-pyrazolo[3,4-b]pyridin-6-yl)piperazin-1-yl)methyl)-1-(oxetan-2-ylmethyl)-1H-benzo[d]imidazole-6-carboxylate (30e)

To the solution of 3-fluoro-4-((6-(piperazin-1-yl)-1H-pyrazolo[3,4-b]pyridin-1-yl)methyl)benzonitrile (30d, 104 mg, 278.95 umol, 1 eq, HCl) and (S)-methyl 2-(chloromethyl)-1-(oxetan-2-ylmethyl)-1H-benzo[d]imidazole-6-carboxylate (1k, 98.66 mg, 334.74 umol, 1.2 eq) in CH3CN (10 mL) was added K2CO3 (192.76 mg, 1.39 mmol, 5 eq) at 20° C. Then the mixture was stirred at 50° C. for 6 hours. TLC (petroleum ether:ethyl acetate=0:1,) showed 30d was consumed and one new major spot was formed. The mixture was filtered and the filtrate was concentrated in vacuo. The residue was purified by column chromatography (SiO2, petroleum ether:ethyl acetate=80:1 to 20:1) to give 30e as a light yellow solid. 1H NMR (400 MHz, MeOD-d4) δ 8.36 (d, J=1.0 Hz, 1H), 7.97 (dd, J=1.6, 8.4 Hz, 1H), 7.87 (d, J=9.0 Hz, 1H), 7.83 (s, 1H), 7.69 (d, J=8.6 Hz, 1H), 7.57 (dd, J=1.6, 9.4 Hz, 1H), 7.45 (dd, J=1.2, 7.8 Hz, 1H), 7.18 (t, J=7.6 Hz, 1H), 6.79 (d, J=9.0 Hz, 1H), 5.64 (s, 2H), 5.27 (dd, J=2.4, 7.2 Hz, 1H), 4.94-4.88 (m, 1H), 4.79-4.71 (m, 1H), 4.68-4.60 (m, 1H), 4.46 (td, J=5.8, 9.2 Hz, 1H), 4.03 (s, 1H), 3.96-3.92 (m, 4H), 3.72 (t, J=5.0 Hz, 4H), 2.86-2.75 (m, 1H), 2.69-2.58 (m, 4H), 2.57-2.47 (m, 1H).

### (S)-2-((4-(1-(4-cyano-2-fluorobenzyl)-1H-pyrazolo[3,4-b]pyridin-6-yl)piperazin-1-yl)methyl)-1-(oxetan-2-ylmethyl)-1H-benzo[d]imidazole-6-carboxylic acid (Compound 30)

LiOH·H2O (8.82 mg, 210.21 umol, 1 eq) was added to the solution of (S)-methyl 2-((4-(1-(4-cyano-2-fluorobenzyl)-1H-pyrazolo[3,4-b]pyridin-6-yl)piperazin-1-yl)methyl)-1-(oxetan-2-ylmethyl)-1H-benzo[d]imidazole-6-carboxylate (30e, 125 mg, 210.21 umol, 1 eq) in THF (8.4 mL) and H2O (3.6 mL) at 20° C. Then the solution was stirred at 20° C. for 8 hours. TLC (dichloromethane:methanol=10:1) showed most of 30e was remained. LiOH·H2O (7.06 mg, 168.17 umol, 0.8 eq) was added to the reaction mixture at 20° C. Then the mixture was stirred at 20° C. for another 16 hours. TLC (dichloromethane:methanol=10:1) showed 30e was consumed and one major new spot was formed. The mixture was adjusted to pH=3 with HOAc, and extracted with ethyl acetate (10 mL*3). The combined ethyl acetate was washed with brine (15 mL), dried over anhydrous Na2SO4, filtered and concentrated. The residue was purified by prep-TLC (dichloromethane:methanol=10:1) to give Compound 30 as white solid. MS mass calculated for [M+1]+(C31H29FN803) requires m/z 581.2, LCMS found m/z 581.3. 1H NMR (400 MHz, MeOD-d4) δ 8.34 (s, 1H), 7.98 (d, J=8.6 Hz, 1H), 7.87 (d, J=9.0 Hz, 1H), 7.82 (s, 1H), 7.68 (d, J=8.6 Hz, 1H), 7.56 (d, J=9.2 Hz, 1H), 7.45 (d, J=8.0 Hz, 1H), 7.19 (t, J=7.6 Hz, 1H), 6.79 (d, J=9.0 Hz, 1H), 5.63 (s, 2H), 5.32-5.22 (m, 1H), 4.94-4.88 (m, 1H), 4.78-4.70 (m, 1H), 4.68-4.59 (m, 1H), 4.47 (td, J=5.8, 9.0 Hz, 1H), 4.10-4.00 (m, 1H), 3.97-3.90 (m, 1H), 3.72 (br t, J=4.6 Hz, 4H), 2.87-2.76 (m, 1H), 2.70-2.58 (m, 4H), 2.58-2.47 (m, 1H).

### Example 31 (General Procedure EE)

### (S)-2-((4-(2-benzyl-2H-pyrazolo[3,4-b]pyridin-6-yl)piperazin-1-yl)methyl)-1-(oxetan-2-ylmethyl)-1H-benzo[d]imidazole-6-carboxylic acid

The title compound was prepared according to Scheme 22. This General Procedure EE exemplifies Scheme 22 and provides particular synthetic details as applied to the title compound.

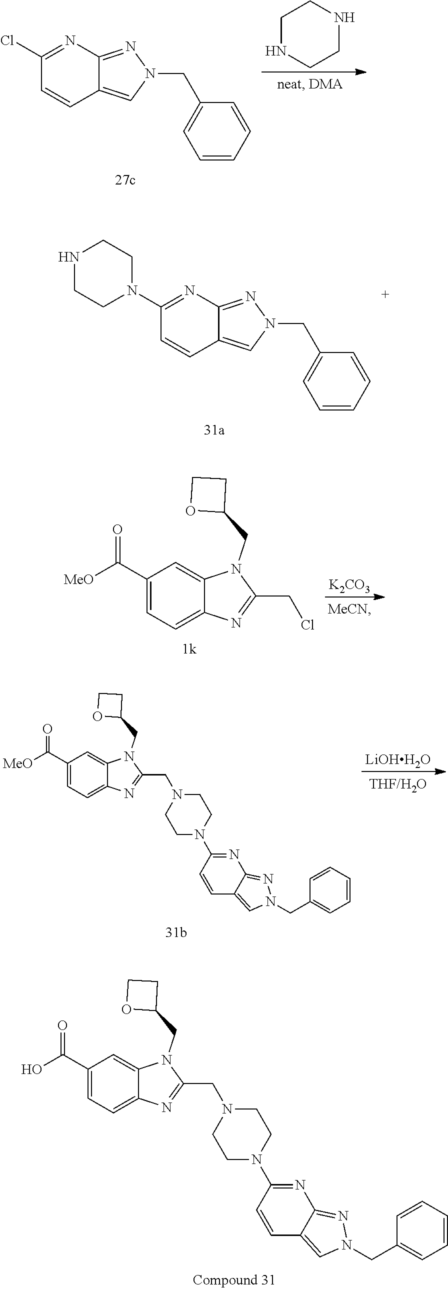

### 2-benzyl-6-(piperazin-1-yl)-2H-pyrazolo[3,4-b]pyridine (31a)

To a solution of 2-benzyl-6-chloro-2H-pyrazolo[3,4-b]pyridine (27c, 200 mg, 820.71 umol, 1 eq) in DMA (1 mL) was added piperazine (353.46 mg, 4.10 mmol, 5 eq). The mixture was stirred at 100° C. for 16 hours. LCMS showed desired mass was detected. The reaction mixture was concentrated under reduced pressure. The residue was diluted with H2O (20 mL) and extracted with MTBE (60 mL). The combined organic layers were washed with brine (5 mL), dried over Na2SO4, filtered and concentrated under reduced pressure. The residue was purified by prep-TLC (SiO2, Ethyl acetate:Methanol=10:1) to give 31a as a white solid. 1H NMR (400 MHz, CDCl3-d) δ 7.66 (d, J=9.4 Hz, 1H), 7.56 (s, 1H), 7.34-7.08 (m, 5H), 6.68-6.58 (m, 1H), 5.39 (s, 2H), 3.72-3.53 (m, 4H), 2.99-2.82 (m, 4H).

### (S)-methyl 2-((4-(2-benzyl-2H-pyrazolo[3,4-b]pyridin-6-yl)piperazin-1-yl)methyl)-1-(oxetan-2-ylmethyl)-1H-benzo[d]imidazole-6-carboxylate (31b)

To a solution of 2-benzyl-6-(piperazin-1-yl)-2H-pyrazolo[3,4-b]pyridine (31a, 50 mg, 170.44 umol, 1 eq) and methyl 2-(chloromethyl)-3-[[(2S)-oxetan-2-yl] methyl] benzimidazole-5-carboxylate (1k, 50.23 mg, 170.44 umol, 1 eq) in CH3CN (1 mL) was added K2CO3 (117.78 mg, 852.18 umol, 5 eq). The mixture was stirred at 60° C. for 16 hours. LCMS showed desired mass was detected. The reaction mixture was concentrated under reduced pressure to remove solvent. The residue was purified by prep-TLC (SiO2, Ethyl acetate:Methanol=10:1) to give 31b as a white solid. 1H NMR (400 MHz, CDCl3-d) δ 8.18 (s, 1H), 7.99 (d, J=8.6 Hz, 1H), 7.75 (dd, J=8.8, 15.8 Hz, 2H), 7.63 (s, 1H), 7.33 (s, 5H), 6.68 (d, J=9.2 Hz, 1H), 5.46 (s, 2H), 5.33-5.18 (m, 1H), 4.82-4.57 (m, 3H), 4.41 (td, J=6.0, 9.0 Hz, 1H), 4.05-3.98 (m, 2H), 3.96 (s, 3H), 3.79-3.66 (m, 4H), 2.84-2.60 (m, 5H), 2.55-2.42 (m, 1H).

### (S)-2-((4-(2-benzyl-2H-pyrazolo[3,4-b]pyridin-6-yl)piperazin-1-yl)methyl)-1-(oxetan-2-ylmethyl)-1H-benzo[d]imidazole-6-carboxylic acid (Compound 31)

To a solution of (S)-methyl 2-((4-(2-benzyl-2H-pyrazolo[3,4-b]pyridin-6-yl)piperazin-1-yl)methyl)-1-(oxetan-2-ylmethyl)-1H-benzo[d]imidazole-6-carboxylate (31b, 70 mg, 126.89 umol, 1 eq) in THF (0.7 mL) and H2O (0.3 mL) was added LiOH·H2O (15.97 mg, 380.68 umol, 3 eq). The mixture was stirred at 15° C. for 24 hr. LCMS showed desired mass was detected. The reaction mixture was added citric acid until pH=7, and then concentrated under reduced pressure to give a residue, then added H2O (1 mL), and then filtered to give 31 as a white solid. MS mass calculated for [M+H]+ (C30H31N7O3) requires m/z 538.2, LCMS found m/z 538.3; 1H NMR (400 MHz, MeOD-d4) δ 8.39-8.28 (m, 1H), 8.00 (s, 2H), 7.90-7.82 (m, 1H), 7.68 (br d, J=8.6 Hz, 1H), 7.31 (br d, J=5.0 Hz, 5H), 6.89-6.80 (m, 1H), 5.47 (s, 2H), 5.32-5.21 (m, 1H), 4.98-4.88 (m, 1H), 4.79-4.69 (m, 1H), 4.69-4.58 (m, 1H), 4.53-4.40 (m, 1H), 4.11-3.89 (m, 2H), 3.71 (br s, 4H), 2.87-2.73 (m, 1H), 2.65 (br d, J=4.4 Hz, 4H), 2.58-2.46 (m, 1H).

### Example 32 (General Procedure FF)

### (S)-2-((4-(3-(benzyloxy)-1,2,4-thiadiazol-5-yl)piperazin-1-yl)methyl)-1-(oxetan-2-ylmethyl)-1H-benzo[d]imidazole-6-carboxylic acid

The title compound was prepared according to Scheme 23. This General Procedure FF exemplifies Scheme 23 and provides particular synthetic details as applied to the title compound.

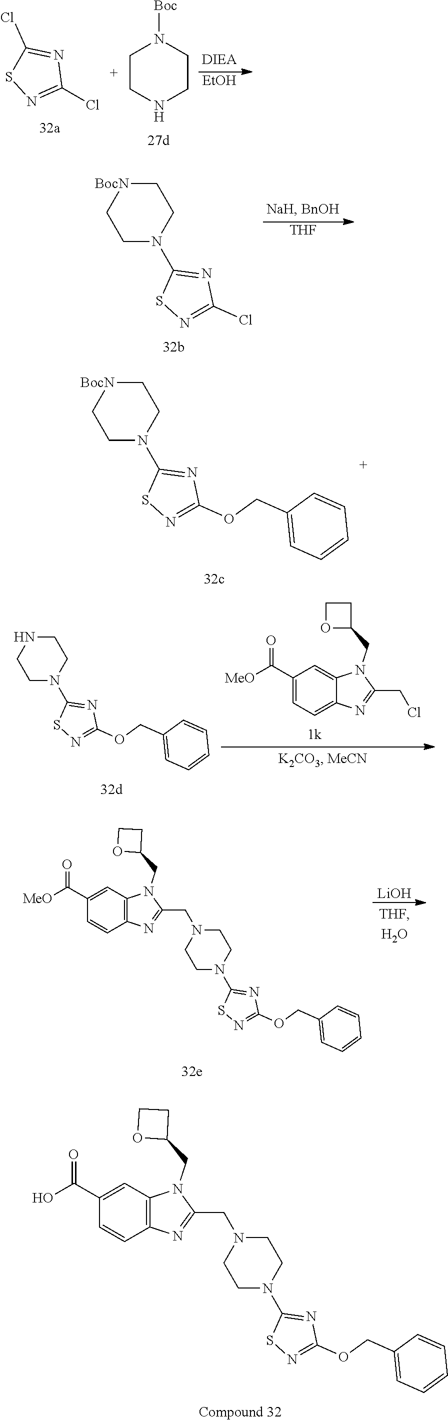

### Tert-butyl 4-(3-chloro-1,2,4-thiadiazol-5-yl)piperazine-1-carboxylate (32b)

A mixture of 3,5-dichloro-1,2,4-thiadiazole (32a, 300 mg, 1.94 mmol, 1 eq), tert-butyl piperazine-1-carboxylate (27d, 396.52 mg, 2.13 mmol, 1.1 eq) and DIEA (800.44 mg, 6.19 mmol, 1.08 mL, 3.2 eq) in EtOH (5 mL) was degassed and purged with N2 3 times. Then the mixture was stirred at 20° C. for 1 hour under N2 atmosphere. LCMS showed 32a was consumed completely and desired mass was detected. The mixture was diluted with H2O (20 mL) and extracted with Ethyl acetate (60 mL *2). The combined organic layers were washed with brine (30 mL*3), dried over with anhydrous Na2SO4, filtered and concentrated under reduced pressure. The residue was purified by column chromatography (SiO2, Petroleum ether:Ethyl acetate=5:1 to 1:1) to give 32b as a white solid. 1H NMR (400 MHz, CDCl3-d) δ 1.49 (s, 9H), 3.61-3.49 (m, 8H).

3-(benzyloxy)-5-(piperazin-1-yl)-1,2,4-thiadiazole (32d) NaH (196.85 mg, 4.92 mmol, 60% purity, 5 eq) was added in BnOH (1.06 g, 9.84 mmol, 1.02 mL, 10 eq) at 20° C. under N2. The mixture was stirred at 20° C. for 1 hour. Then a solution of tert-butyl 4-(3-chloro-1,2,4-thiadiazol-5-yl)piperazine-1-carboxylate (32b, 300 mg, 984.27 umol, 1 eq) in THF (1 mL) was added in the mixture under N2. The mixture was stirred at 80° C. for 15 hours. LCMS showed 32b was consumed completely and mass of 32c and 32d were detected. The reaction mixture was quenched by addition of saturated NH4Cl solution (30 mL) at 20° C. The aqueous phase was extracted with ethyl acetate (30 mL*2). The combined organic layers were washed with brine (20 mL), dried over with anhydrous Na2SO4, filtered and concentrated in vacuo. The residue was purified by prep-TLC (Petroleum ether:Ethyl acetate=3:1) to give 32d as a white solid. 1H NMR (400 MHz, CDCl3-d) δ 7.56-7.41 (m, 2H), 7.41-7.30 (m, 3H), 5.36 (s, 2H), 3.47 (br d, J=4.53 Hz, 4H), 3.08-2.92 (m, 4H).

### (S)-methyl 2-((4-(3-(benzyloxy)-1,2,4-thiadiazol-5-yl)piperazin-1-yl)methyl)-1-(oxetan-2-ylmethyl)-1H-benzo[d]imidazole-6-carboxylate (32e)

To a mixture of 3-(benzyloxy)-5-(piperazin-1-yl)-1,2,4-thiadiazole (32d, 75 mg, 239.76 umol, 1 eq, HCl) in CH3CN (2 mL) was added K2CO3 (132.55 mg, 959.03 umol, 4 eq) at 20° C. under N2 for 0.5 hour. Then (S)-methyl 2-(chloromethyl)-1-(oxetan-2-ylmethyl)-1H-benzo[d]imidazole-6-carboxylate (1k, 77.73 mg, 263.73 umol, 1.1 eq) was added in the mixture, and the mixture was stirred at 80° C. for 2.5 hours. LCMS showed 32d was remained and desired mass was detected. The mixture was diluted with H2O (10 mL) and extracted with Ethyl acetate (30 mL *2). The combined organic layers were washed with brine (15 mL*3), dried over anhydrous Na2SO4, filtered and concentrated under reduced pressure. The residue was purified by prep-TLC (Ethyl acetate:Methanol=10:1) to give 32e as a yellow solid. 1H NMR (400 MHz, CDCl3-d) δ 8.14 (d, J=0.8 Hz, 1H), 7.99 (dd, J=8.4, 1.4 Hz, 1H), 7.77 (d, J=8.4 Hz, 1H), 7.45 (d, J=6.8 Hz, 2H), 7.39-7.30 (m, 3H), 5.36 (s, 2H), 5.26-5.18 (m, 1H), 4.68-4.59 (m, 3H), 4.35 (dt, J=9.2, 5.8 Hz, 1H), 4.18-4.01 (m, 2H), 3.96 (s, 3H), 3.51 (br s, 4H), 2.79-2.71 (m, 1H), 2.68 (t, J=5.0 Hz, 4H), 2.49-2.39 (m, 1H).

### (S)-2-((4-(3-(benzyloxy)-1,2,4-thiadiazol-5-yl)piperazin-1-yl)methyl)-1-(oxetan-2-ylmethyl)-1H-benzo[d]imidazole-6-carboxylic acid (Compound 32)

To a solution of (S)-methyl 2-((4-(3-(benzyloxy)-1,2,4-thiadiazol-5-yl)piperazin-1-yl)methyl)-1-(oxetan-2-ylmethyl)-1H-benzo[d]imidazole-6-carboxylate (32e, 90 mg, 168.34 umol, 1 eq) in THF (2.1 mL) and H2O (0.9 mL) was added LiOH·H2O (28.25 mg, 673.36 umol, 75.67 uL, 4 eq) at 25° C. The mixture was stirred at 25° C. for 16 hours. TLC (Ethyl acetate:Methanol=10:1, Rf=0.5) showed 32e was consumed completely and desired mass was detected in LCMS. The mixture was adjusted to pH=6 with Citric acid (1 M). Then the mixture was concentrated under reduced pressure to remove THF. The residue was purified by Prep-HPLC (column: Waters Xbridge BEH C18 250*50 mm*10 um; mobile phase: [water (10 mM NH4HCO3)-ACN]; B %: 20%-50%, 8 min) to give 32 as a white solid. MS mass calculated for [M+H]+ (C26H28N6O4S) requires m/z 521.2, LCMS found m/z 521.3; 1H NMR (400 MHz, MeOD-d4) δ 8.33 (s, 1H), 7.97 (dd, J=8.56, 0.86 Hz, 1H), 7.67 (d, J=8.44 Hz, 1H), 7.45-7.28 (m, 5H), 5.32 (s, 2H), 5.25 (br dd, J=7.21, 2.08 Hz, 1H), 4.92-4.88 (m, 1H), 4.76-4.68 (m, 1H), 4.67-4.60 (m, 1H), 4.45 (dt, J=9.14, 5.88 Hz, 1H), 4.10-4.02 (m, 1H), 4.00-3.94 (m, 1H), 3.51 (br s, 4H), 2.85-2.75 (m, 1H), 2.72-2.60 (m, 4H), 2.57-2.46 (m, 1H).

### Example 33

### (S)-1-(oxetan-2-ylmethyl)-2-((4-(6-((tetrahydro-2H-pyran-4-yl)methoxy)pyridin-2-yl)piperazin-1-yl)methyl)-1H-benzo[d]imidazole-6-carboxylic acid

The title compound was prepared and can be prepared similarly following the procedures exemplified by General Procedure A.

### (S)-1-(oxetan-2-ylmethyl)-2-((4-(6-((tetrahydro-2H-pyran-4-yl)methoxy)pyridin-2-yl)piperazin-1-yl)methyl)-1H-benzo[d]imidazole-6-carboxylic acid (Compound 33)

1H NMR (400 MHz, MeOD-d4) δ 8.34 (s, 1H), 7.98 (d, J=8.4 Hz, 1H), 7.68 (d, J=8.4 Hz, 1H), 7.41 (t, J=7.9 Hz, 1H), 6.25 (d, J=8.1 Hz, 1H), 6.04 (d, J=7.8 Hz, 1H), 5.37-5.13 (m, 1H), 4.91 (br d, J=7.2 Hz, 1H), 4.78-4.71 (m, 1H), 4.68-4.58 (m, 1H), 4.47 (td, J=5.9, 9.1 Hz, 1H), 4.12-4.01 (m, 3H), 4.00-3.87 (m, 3H), 3.53 (br t, J=4.6 Hz, 4H), 3.43 (dt, J=2.0, 11.8 Hz, 2H), 2.85-2.76 (m, 1H), 2.71-2.59 (m, 4H), 2.53 (br s, 1H), 2.09-1.93 (m, 1H), 1.71 (br d, J=12.5 Hz, 2H), 1.43-1.38 (m, 2H).

### Example 34

### (S)-2-((4-(6-((3,3-difluoro-1-methylcyclobutyl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((3,3-difluoro-1-methylcyclobutyl)methoxy)pyridin-2-yl)piperazin-1-yl)methyl)-1-(oxetan-2-ylmethyl)-1H-benzo[d]imidazole-6-carboxylic acid (Compound 34)

1H NMR (400 MHz, MeOD-d4) δ 8.34 (s, 1H), 7.97 (dd, J=1.3, 8.6 Hz, 1H), 7.67 (d, J=8.4 Hz, 1H), 7.42 (s, 1H), 6.27 (d, J=7.9 Hz, 1H), 6.07 (d, J=7.9 Hz, 1H), 5.32-5.22 (m, 1H), 4.92 (br s, 1H), 4.73 (br dd, J=2.3, 15.3 Hz, 1H), 4.66-4.62 (m, 1H), 4.50-4.41 (m, 1H), 4.16 (s, 2H), 4.10-4.00 (m, 1H), 3.98-3.89 (m, 1H), 3.53 (br t, J=4.7 Hz, 4H), 2.86-2.73 (m, 1H), 2.70-2.45 (m, 7H), 2.27 (br d, J=11.7 Hz, 2H), 1.31 (s, 3H).

### Example 35

### (S)-1-(oxetan-2-ylmethyl)-2-((4-(6-(2-(oxetan-3-yl)ethoxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-(2-(oxetan-3-yl)ethoxy)pyridin-2-yl)piperazin-1-yl)methyl)-1H-benzo[d]imidazole-6-carboxylic acid (Compound 35)

1H NMR (400 MHz, METHANOL-d4) δ 8.32 (s, 1H), 7.98 (d, J=8.4 Hz, 1H), 7.67 (d, J=8.4 Hz, 1H), 7.41 (t, J=7.9 Hz, 1H), 6.25 (d, J=7.9 Hz, 1H), 6.00 (d, J=7.7 Hz, 1H), 5.33-5.22 (m, 1H), 4.93 (br s, 1H), 4.80 (br d, J=1.8 Hz, 1H), 4.77-4.72 (m, 1H), 4.67-4.62 (m, 3H), 4.51-4.43 (m, 2H), 4.23 (t, J=6.2 Hz, 2H), 4.11-4.01 (m, 1H), 3.98-3.88 (m, 1H), 3.52 (br t, J=4.7 Hz, 4H), 3.25-3.14 (m, 1H), 2.86-2.75 (m, 1H), 2.63 (br d, J=5.1 Hz, 4H), 2.54 (br d, J=9.0 Hz, 1H), 2.17-2.03 (m, 2H).

### Example 36

### (S)-2-((4-(6-((4,4-difluorocyclohexyl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

(S)-2-((4-(6-((4,4-difluorocyclohexyl)methoxy)pyridin-2-yl)piperazin-1-yl)methyl)-1-(oxetan-2-ylmethyl)-1H-benzo[d]imidazole-6-carboxylic acid (Compound 36).

1H NMR (400 MHz, MeOD-d4) δ 8.33 (s, 1H), 7.98 (d, J=8.6 Hz, 1H), 7.67 (d, J=8.2 Hz, 1H), 7.41 (t, J=7.9 Hz, 1H), 6.25 (d, J=7.9 Hz, 1H), 6.04 (d, J=7.9 Hz, 1H), 5.28 (br d, J=4.9 Hz, 1H), 4.91 (br d, J=6.8 Hz, 1H), 4.77-4.71 (m, 1H), 4.67-4.61 (m, 1H), 4.47 (td, J=5.9, 9.4 Hz, 1H), 4.09 (d, J=6.0 Hz, 2H), 4.07-4.01 (m, 1H), 3.97-3.89 (m, 1H), 3.52 (br t, J=4.6 Hz, 4H), 2.88-2.74 (m, 1H), 2.63 (q, J=4.6 Hz, 4H), 2.56-2.46 (m, 1H), 2.04 (br s, 2H), 1.94-1.64 (m, 5H), 1.46-1.30 (m, 2H).

### Example 37

### (S)-2-((4-(6-((1-methylpiperidin-4-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C. 37 (S)-2-((4-(6-((1-methylpiperidin-4-yl)methoxy)pyridin-2-yl)piperazin-1-yl)methyl)-1-(oxetan-2-ylmethyl)-1H-benzo[d]imidazole-6-carboxylic acid (Compound 37).

1H NMR (400 MHz, MeOD-d4) δ 8.20 (s, 1H), 7.95 (dd, J=1.4, 8.5 Hz, 1H), 7.59 (d, J=8.6 Hz, 1H), 7.41 (t, J=7.9 Hz, 1H), 6.27 (d, J=8.2 Hz, 1H), 6.02 (d, J=7.7 Hz, 1H), 5.27 (br d, J=4.9 Hz, 1H), 4.95-4.89 (m, 1H), 4.76-4.69 (m, 1H), 4.66-4.61 (m, 1H), 4.45 (td, J=6.0, 9.1 Hz, 1H), 4.14 (d, J=5.7 Hz, 2H), 4.06-3.86 (m, 2H), 3.51 (br s, 4H), 3.41-3.34 (m, 2H), 2.87-2.75 (m, 3H), 2.73 (s, 3H), 2.61 (br s, 4H), 2.56-2.47 (m, 1H), 2.00 (br d, J=14.1 Hz, 3H), 1.67-1.45 (m, 2H).

### Example 38

### (S)-2-((4-(6-((1-acetylpiperidin-4-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((1-acetylpiperidin-4-yl)methoxy)pyridin-2-yl)piperazin-1-yl)methyl)-1-(oxetan-2-ylmethyl)-1H-benzo[d]imidazole-6-carboxylic acid (Compound 38)

1H NMR (400 MHz, MeOD-d4) δ 8.35 (s, 1H), 7.98 (d, J=8.6 Hz, 1H), 7.68 (d, J=8.4 Hz, 1H), 7.41 (t, J=8.0 Hz, 1H), 6.26 (d, J=8.1 Hz, 1H), 6.05 (d, J=7.8 Hz, 1H), 5.37-5.19 (m, 1H), 4.94-4.88 (m, 1H), 4.78-4.70 (m, 1H), 4.69-4.60 (m, 1H), 4.54 (br d, J=13.2 Hz, 1H), 4.47 (td, J=6.0, 9.1 Hz, 1H), 4.10 (dd, J=1.3, 6.4 Hz, 2H), 4.08-4.02 (m, 1H), 3.94 (br d, J=13.7 Hz, 2H), 3.53 (br t, J=4.8 Hz, 4H), 3.13 (br d, J=2.2 Hz, 1H), 2.86-2.75 (m, 1H), 2.71-2.59 (m, 5H), 2.58-2.47 (m, 1H), 2.09 (s, 4H), 1.94-1.78 (m, 2H), 1.39-1.15 (m, 2H).

### Example 39

### (S)-1-(oxetan-2-ylmethyl)-2-((4-(6-((1-phenylazetidin-3-yl)oxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-((1-phenylazetidin-3-yl)oxy)pyridin-2-yl)piperazin-1-yl)methyl)-1H-benzo[d]imidazole-6-carboxylic acid (Compound 39)

1H NMR (400 MHz, MeOD-d4) δ 8.35 (s, 1H), 7.98 (dd, J=1.3, 8.4 Hz, 1H), 7.68 (d, J=8.6 Hz, 1H), 7.45 (t, J=8.0 Hz, 1H), 7.17 (t, J=7.9 Hz, 2H), 6.77-6.66 (m, 1H), 6.50 (d, J=7.7 Hz, 2H), 6.31 (d, J=8.1 Hz, 1H), 6.09 (d, J=7.8 Hz, 1H), 5.46-5.36 (m, 1H), 5.33-5.22 (m, 1H), 4.93-4.87 (m, 1H), 4.77-4.71 (m, 1H), 4.69-4.60 (m, 1H), 4.47 (td, J=5.9, 9.1 Hz, 1H), 4.33-4.23 (m, 2H), 4.11-4.02 (m, 1H), 4.00-3.89 (m, 1H), 3.76 (dd, J=4.6, 8.4 Hz, 2H), 3.55 (br t, J=4.8 Hz, 4H), 2.87-2.75 (m, 1H), 2.72-2.60 (m, 4H), 2.59-2.45 (m, 1H).

### Example 40

### (S)-1-(oxetan-2-ylmethyl)-2-((4-(6-(pyridin-2-ylmethoxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-(pyridin-2-ylmethoxy)pyridin-2-yl)piperazin-1-yl)methyl)-1H-benzo[d]imidazole-6-carboxylic acid (Compound 40)

1H NMR (400 MHz, MeOD-d4) δ 8.48 (d, J=4.8 Hz, 1H), 8.36 (s, 1H), 7.99 (dd, J=1.3, 8.5 Hz, 1H), 7.82 (dt, J=1.6, 7.7 Hz, 1H), 7.70 (d, J=8.4 Hz, 1H), 7.54-7.42 (m, 2H), 7.32 (dd, J=5.3, 7.0 Hz, 1H), 6.29 (d, J=8.1 Hz, 1H), 6.20 (d, J=7.9 Hz, 1H), 5.40 (s, 2H), 5.27 (br dd, J=2.4, 7.2 Hz, 1H), 4.94-4.88 (m, 1H), 4.78-4.71 (m, 1H), 4.69-4.60 (m, 1H), 4.48 (td, J=5.9, 9.2 Hz, 1H), 4.07-3.99 (m, 1H), 3.95-3.87 (m, 1H), 3.46 (br t, J=4.8 Hz, 4H), 2.80 (br s, 1H), 2.63-2.46 (m, 5H).

### Example 41

### (S)-2-((4-(6-((4-cyanotetrahydro-2H-pyran-4-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((4-cyanotetrahydro-2H-pyran-4-yl)methoxy)pyridin-2-yl)piperazin-1-yl)methyl)-1-(oxetan-2-ylmethyl)-1H-benzo[d]imidazole-6-carboxylic acid (Compound 41)

1H NMR (400 MHz, MeOD-d4) δ ppm 8.34 (s, 1H), 7.97 (dd, J=1.3, 8.4 Hz, 1H), 7.68 (d, J=8.6 Hz, 1H), 7.45 (t, J=7.9 Hz, 1H), 6.31 (d, J=8.1 Hz, 1H), 6.12 (d, J=7.8 Hz, 1H), 5.32-5.22 (m, 1H), 4.94-4.86 (m, 1H), 4.77-4.69 (m, 1H), 4.63 (s, 1H), 4.46 (td, J=6.0, 9.1 Hz, 1H), 4.33 (s, 2H), 4.09-4.01 (m, 1H), 4.00-3.89 (m, 3H), 3.73-3.63 (m, 2H), 3.54 (br t, J=4.8 Hz, 4H), 2.86-2.74 (m, 1H), 2.70-2.58 (m, 4H), 2.57-2.46 (m, 1H), 1.95 (br d, J=13.1 Hz, 2H), 1.84-1.72 (m, 2H).

### Example 42

### (S)-1-(oxetan-2-ylmethyl)-2-((4-(6-(pyridin-4-ylmethoxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-(pyridin-4-ylmethoxy)pyridin-2-yl)piperazin-1-yl)methyl)-1H-benzo[d]imidazole-6-carboxylic acid (Compound 42)

1H NMR (400 MHz, MeOD-d4) δ 8.48-8.44 (m, 2H), 8.34 (s, 1H), 7.98 (dd, J=1.4, 8.5 Hz, 1H), 7.68 (d, J=8.6 Hz, 1H), 7.50-7.42 (m, 3H), 6.29 (d, J=8.1 Hz, 1H), 6.19 (d, J=7.8 Hz, 1H), 5.39 (s, 2H), 5.30-5.22 (m, 1H), 4.95-4.89 (m, 1H), 4.70-4.70 (m, 1H), 4.76-4.69 (m, 1H), 4.67-4.59 (m, 1H), 4.46 (td, J=5.9, 9.0 Hz, 1H), 4.05-3.99 (m, 1H), 3.94-3.87 (m, 1H), 3.45 (br t, J=4.6 Hz, 4H), 2.85-2.73 (m, 1H), 2.61-2.46 (m, 5H).

### Example 43

### (S)-2-((4-(6-((2-oxaspiro[3.3]heptan-6-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((2-oxaspiro[3.3]heptan-6-yl)methoxy)pyridin-2-yl)piperazin-1-yl)methyl)-1-(oxetan-2-ylmethyl)-1H-benzo[d]imidazole-6-carboxylic acid (Compound 43)

1H NMR (400 MHz, CDCl3-d) δ 8.24 (s, 1H), 8.06 (d, J=8.6 Hz, 1H), 7.82 (d, J=8.6 Hz, 1H), 7.40 (t, J=7.8 Hz, 1H), 6.15 (d, J=8.0 Hz, 1H), 6.06 (d, J=7.8 Hz, 1H), 5.29-5.22 (m, 1H), 4.80-4.69 (m, 4H), 4.69-4.61 (m, 3H), 4.42 (td, J=5.8, 9.0 Hz, 1H), 4.13 (d, J=6.0 Hz, 2H), 4.04 (s, 2H), 3.51 (br s, 4H), 2.81-2.72 (m, 1H), 2.68 (br t, J=4.6 Hz, 4H), 2.58-2.44 (m, 2H), 2.44-2.34 (m, 2H), 2.08 (dd, J=6.8, 12.8 Hz, 2H).

### Example 44

### (S)-2-((4-(6-(2-cyclohexylethoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-(2-cyclohexylethoxy)pyridin-2-yl)piperazin-1-yl)methyl)-1-(oxetan-2-ylmethyl)-1H-benzo[d]imidazole-6-carboxylic acid (Compound 44)

1H NMR (400 MHz, MeOH-d4) δ 8.34 (s, 1H), 8.02-7.95 (m, 1H), 7.67 (d, J=8.4 Hz, 1H), 7.40 (t, J=8.0 Hz, 1H), 6.24 (d, J=8.2 Hz, 1H), 6.02 (d, J=7.8 Hz, 1H), 5.34-5.22 (m, 1H), 4.95-4.86 (m, 1H), 4.77-4.71 (m, 1H), 4.68-4.60 (m, 1H), 4.47 (td, J=6.0, 9.0 Hz, 1H), 4.24 (t, J=6.8 Hz, 2H), 4.03 (s, 1H), 3.96-3.85 (m, 1H), 3.53 (br t, J=4.8 Hz, 4H), 2.86-2.76 (m, 1H), 2.68-2.58 (m, 4H), 2.58-2.50 (m, 1H), 1.80-1.66 (m, 4H), 1.61 (q, J=6.8 Hz, 2H), 1.52-1.40 (m, 1H), 1.33-1.14 (m, 3H), 1.04-0.90 (m, 2H).

### Example 45

### (S)-1-(oxetan-2-ylmethyl)-2-((4-(6-((3-phenyloxetan-3-yl)oxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-((3-phenyloxetan-3-yl)oxy)pyridin-2-yl)piperazin-1-yl)methyl)-1H-benzo[d]imidazole-6-carboxylic acid (Compound 45)

1H NMR (400 MHz, MeOH-d4) δ =8.34 (s, 1H), 8.02-7.95 (m, 1H), 7.69 (d, J=8.4 Hz, 1H), 7.53 (d, J=7.6 Hz, 2H), 7.46 (t, J=8.0 Hz, 1H), 7.38-7.28 (m, 2H), 7.28-7.16 (m, 1H), 6.23 (dd, J=5.0, 7.8 Hz, 2H), 5.24 (br d, J=5.4 Hz, 1H), 5.10 (d, J=7.4 Hz, 2H), 4.95 (d, J=7.4 Hz, 2H), 4.84-4.80 (m, 1H), 4.75-4.61 (m, 2H), 4.51-4.41 (m, 1H), 3.98-3.79 (m, 2H), 3.20 (br t, J=5.0 Hz, 4H), 2.84-2.72 (m, 1H), 2.61-2.46 (m, 1H), 2.46-2.29 (m, 4H).

### Example 46

### (S)-2-((4-(6-((2-oxaspiro[3.5]nonan-7-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((2-oxaspiro[3.5]nonan-7-yl)methoxy)pyridin-2-yl)piperazin-1-yl)methyl)-1-(oxetan-2-ylmethyl)-1H-benzo[d]imidazole-6-carboxylic acid (Compound 46)

1H NMR (400 MHz, MeOH-d4) δ =8.35 (s, 1H), 7.98 (dd, J=1.4, 8.6 Hz, 1H), 7.68 (d, J=8.4 Hz, 1H), 7.40 (t, J=7.8 Hz, 1H), 6.24 (d, J=7.8 Hz, 1H), 6.03 (d, J=7.8 Hz, 1H), 5.27 (br d, J=7.0 Hz, 1H), 4.92 (br d, J=7.2 Hz, 1H), 4.78-4.70 (m, 1H), 4.70-4.61 (m, 1H), 4.50-4.45 (m, 1H), 4.45 (s, 2H), 4.36-4.33 (m, 2H), 4.08-4.03 (m, 1H), 4.00 (d, J=6.2 Hz, 2H), 3.98-3.90 (m, 1H), 3.52 (br t, J=4.6 Hz, 4H), 2.87-2.73 (m, 1H), 2.69-2.58 (m, 4H), 2.58-2.48 (m, 1H), 2.15 (br d, J=12.8 Hz, 2H), 1.79 (br d, J=13.0 Hz, 2H), 1.70 (br s, 1H), 1.55-1.42 (m, 2H), 1.13-0.99 (m, 2H).

### Example 47

(S)-1-(oxetan-2-ylmethyl)-2-((4-(6-(oxetan-3-ylmethoxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-(oxetan-3-ylmethoxy)pyridin-2-yl)piperazin-1-yl)methyl)-1H-benzo[d]imidazole-6-carboxylic acid (Compound 47)

1H NMR (400 MHz, MeOH-d4) δ =8.31 (s, 1H), 7.96 (d, J=8.4 Hz, 1H), 7.66 (d, J=8.4 Hz, 1H), 7.42 (t, J=7.8 Hz, 1H), 6.27 (d, J=7.8 Hz, 1H), 6.06 (d, J=7.6 Hz, 1H), 5.36-5.15 (m, 1H), 4.93-4.90 (m, 1H), 4.83 (dd, J=6.2, 8.0 Hz, 2H), 4.77-4.70 (m, 1H), 4.67-4.60 (m, 1H), 4.56 (t, J=6.0 Hz, 2H), 4.49-4.42 (m, 3H), 4.13-3.95 (m, 1H), 3.96-3.88 (m, 1H), 3.53 (br t, J=4.8 Hz, 4H), 3.46-3.36 (m, 1H), 2.86-2.74 (m, 1H), 2.63 (q, J=4.8 Hz, 4H), 2.56-2.47 (m, 1H).

### Example 48

### (S)-2-((4-(6-((7-oxaspiro[3.5]nonan-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((7-oxaspiro[3.5]nonan-2-yl)methoxy)pyridin-2-yl)piperazin-1-yl)methyl)-1-(oxetan-2-ylmethyl)-1H-benzo[d]imidazole-6-carboxylic acid (Compound 48)

1H NMR (400 MHz, MeOH-d4) δ 8.34 (d, J=0.8 Hz, 1H), 7.98 (dd, J=1.4, 8.6 Hz, 1H), 7.68 (d, J=8.6 Hz, 1H), 7.40 (t, J=8.0 Hz, 1H), 6.24 (d, J=8.2 Hz, 1H), 6.03 (d, J=8.0 Hz, 1H), 5.27 (br dd, J=2.4, 7.0 Hz, 1H), 4.91 (br d, J=7.4 Hz, 1H), 4.79-4.70 (m, 1H), 4.68-4.60 (m, 1H), 4.47 (td, J=5.8, 9.0 Hz, 1H), 4.18 (d, J=6.4 Hz, 2H), 4.09-4.01 (m, 1H), 3.98-3.90 (m, 1H), 3.66-3.59 (m, 2H), 3.57-3.47 (m, 6H), 2.88-2.75 (m, 1H), 2.73-2.58 (m, 5H), 2.52 (br dd, J=9.0, 11.2 Hz, 1H), 2.04-1.94 (m, 2H), 1.73-1.62 (m, 4H), 1.60-1.50 (m, 2H).

### Example 49

### (S)-1-(oxetan-2-ylmethyl)-2-((4-(6-(spiro[3.5]nonan-7-yloxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-(spiro[3.5]nonan-7-yloxy)pyridin-2-yl)piperazin-1-yl)methyl)-1H-benzo[d]imidazole-6-carboxylic acid (Compound 49)

1H NMR (400 MHz, MeOH-d4) δ 8.33 (s, 1H), 7.97 (dd, J=1.4, 8.4 Hz, 1H), 7.67 (d, J=8.6 Hz, 1H), 7.38 (t, J=8.0 Hz, 1H), 6.22 (d, J=8.2 Hz, 1H), 5.99 (d, J=7.8 Hz, 1H), 5.33-5.19 (m, 1H), 4.92 (br s, 1H), 4.79-4.70 (m, 1H), 4.69-4.58 (m, 1H), 4.47 (td, J=5.8, 9.0 Hz, 1H), 4.10-4.00 (m, 1H), 3.98-3.88 (m, 1H), 3.50 (br t, J=4.8 Hz, 4H), 2.88-2.74 (m, 1H), 2.70-2.58 (m, 4H), 2.54 (br d, J=9.0 Hz, 1H), 1.94-1.69 (m, 11H), 1.59-1.36 (m, 4H).

### Example 50

### (S)-2-((4-(6-((3,3-difluorocyclobutyl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((3,3-difluorocyclobutyl)methoxy)pyridin-2-yl)piperazin-1-yl)methyl)-1-(oxetan-2-ylmethyl)-1H-benzo[d]imidazole-6-carboxylic acid (Compound 50)

1H NMR (400 MHz, CDCl-d) δ 8.24 (s, 1H), 8.05 (d, J=8.6 Hz, 1H), 7.81 (d, J=8.6 Hz, 1H), 7.41 (t, J=7.8 Hz, 1H), 6.17 (d, J=7.8 Hz, 1H), 6.09 (d, J=7.8 Hz, 1H), 5.25 (br dd, J=2.8, 6.6 Hz, 1H), 4.79-4.62 (m, 3H), 4.42 (td, J=5.8, 9.0 Hz, 1H), 4.28 (d, J=6.4 Hz, 2H), 4.04 (s, 2H), 3.52 (br s, 4H), 2.80-2.56 (m, 8H), 2.53-2.38 (m, 3H).

### Example 51

### (S)-1-(oxetan-2-ylmethyl)-2-((4-(6-(1-phenylcyclobutoxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-(1-phenylcyclobutoxy)pyridin-2-yl)piperazin-1-yl)methyl)-1H-benzo[d]imidazole-6-carboxylic acid (Compound 51)

1H NMR (400 MHz, MeOH-d4) δ 8.44-8.30 (m, 1H), 8.03-7.93 (m, 3H), 7.71 (d, J=8.6 Hz, 1H), 7.60-7.43 (m, 4H), 6.66-6.53 (m, 2H), 5.30-5.27 (m, 1H), 4.78-4.73 (m, 1H), 4.68-4.63 (m, 2H), 4.53-4.44 (m, 1H), 4.10-4.05 (m, 1H), 3.98-3.92 (m, 1H), 3.53 (br s, 1H), 3.33 (td, J=1.6, 3.2 Hz, 4H), 3.08 (t, J=7.2 Hz, 2H), 2.87-2.79 (m, 1H), 2.78-2.70 (m, 2H), 2.63 (br d, J=6.0 Hz, 4H), 2.58-2.48 (m, 1H), 2.12 (t, J=7.4 Hz, 2H).

### Example 52

### (S)-2-((4-(6-((1-(methylsulfonyl)piperidin-4-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((1-(methylsulfonyl)piperidin-4-yl)methoxy)pyridin-2-yl)piperazin-1-yl)methyl)-1-(oxetan-2-ylmethyl)-1H-benzo[d]imidazole-6-carboxylic acid (Compound 52)

1H NMR (400 MHz, MeOH-d4) δ 8.35 (s, 1H), 7.98 (dd, J=1.4, 8.4 Hz, 1H), 7.68 (d, J=8.4 Hz, 1H), 7.42 (t, J=8.0 Hz, 1H), 6.26 (d, J=8.0 Hz, 1H), 6.05 (d, J=7.8 Hz, 1H), 5.36-5.21 (m, 1H), 4.94-4.88 (m, 1H), 4.78-4.71 (m, 1H), 4.69-4.60 (m, 1H), 4.47 (td, J=6.0, 9.0 Hz, 1H), 4.12 (d, J=6.0 Hz, 2H), 4.08-4.02 (m, 1H), 3.97-3.91 (m, 1H), 3.74 (br d, J=11.8 Hz, 2H), 3.53 (br t, J=4.8 Hz, 4H), 2.86-2.70 (m, 6H), 2.69-2.59 (m, 4H), 2.58-2.48 (m, 1H), 1.98-1.84 (m, 3H), 1.41 (br dd, J=2.8, 12.4 Hz, 2H).

### Example 53

### (S)-1-(oxetan-2-ylmethyl)-2-((4-(6-(pyridin-3-ylmethoxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-(pyridin-3-ylmethoxy)pyridin-2-yl)piperazin-1-yl)methyl)-1H-benzo[d]imidazole-6-carboxylic acid (Compound 53)

1H NMR (400 MHz, MeOD-d4) δ 8.59 (d, J=1.4 Hz, 1H), 8.44 (dd, J=1.4, 5.0 Hz, 1H), 8.34 (d, J=0.8 Hz, 1H), 7.98 (dd, J=1.6, 8.6 Hz, 1H), 7.89 (br d, J=7.8 Hz, 1H), 7.68 (d, J=8.6 Hz, 1H), 7.48-7.39 (m, 2H), 6.28 (d, J=8.0 Hz, 1H), 6.14 (d, J=7.8 Hz, 1H), 5.38 (s, 2H), 5.27 (br dd, J=2.4, 7.2 Hz, 1H), 4.94-4.88 (m, 1H), 4.78-4.70 (m, 1H), 4.69-4.60 (m, 1H), 4.47 (td, J=5.8, 9.1 Hz, 1H), 4.08-4.00 (m, 1H), 3.96-3.89 (m, 1H), 3.50 (br t, J=4.8 Hz, 4H), 2.79 (br s, 1H), 2.65-2.47 (m, 5H).

### Example 54

### (S)-2-((4-(6-((1,1-dioxidotetrahydro-2H-thiopyran-4-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((1,1-dioxidotetrahydro-2H-thiopyran-4-yl)methoxy)pyridin-2-yl)piperazin-1-yl)methyl)-1-(oxetan-2-ylmethyl)-1H-benzo[d]imidazole-6-carboxylic acid (Compound 54)

1H NMR (400 MHz, CDCl3-d) δ 8.23 (s, 1H), 8.05 (d, J=8.4 Hz, 1H), 7.81 (d, J=8.4 Hz, 1H), 7.41 (t, J=7.8 Hz, 1H), 6.17 (d, J=7.8 Hz, 1H), 6.08 (d, J=7.8 Hz, 1H), 5.26 (br s, 1H), 4.79-4.62 (m, 3H), 4.44-4.38 (m, 1H), 4.15 (br d, J=5.4 Hz, 2H), 4.04 (s, 2H), 3.51 (br s, 4H), 3.11 (br d, J=12.8 Hz, 2H), 3.06-2.94 (m, 2H), 2.81-2.73 (m, 1H), 2.72-2.64 (m, 4H), 2.49 (br d, J=7.8 Hz, 1H), 2.25 (br d, J=10.8 Hz, 2H), 2.05-1.96 (m, 3H).

### Example 55

### (S)-2-((4-(6-(benzylamino)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(benzylamino)pyridin-2-yl)piperazin-1-yl)methyl)-1-(oxetan-2-ylmethyl)-1H-benzo[d]imidazole-6-carboxylic acid (Compound 55)

1H NMR (400 MHz, MeOD-d4) δ 8.33 (s, 1H), 7.99 (dd, J=1.4, 8.6 Hz, 1H), 7.67 (d, J=8.4 Hz, 1H), 7.42-7.10 (m, 6H), 5.95 (d, J=7.8 Hz, 1H), 5.87 (d, J=7.8 Hz, 1H), 5.36-5.20 (m, 1H), 4.94-4.91 (m, 1H), 4.81-4.70 (m, 1H), 4.70-4.60 (m, 1H), 4.54-4.42 (m, 3H), 4.09-3.99 (m, 1H), 3.97-3.87 (m, 1H), 3.45 (br t, J=4.6 Hz, 4H), 2.80 (br s, 1H), 2.67-2.47 (m, 5H).

### Example 56

### (S)-2-((4-(6-((1-(methylsulfonyl)azetidin-3-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((1-(methylsulfonyl)azetidin-3-yl)methoxy)pyridin-2-yl)piperazin-1-yl)methyl)-1-(oxetan-2-ylmethyl)-1H-benzo[d]imidazole-6-carboxylic acid (Compound 56)

1H NMR (400 MHz, MeOD-d4) δ 8.34 (s, 1H), 7.99 (dd, J=1.4, 8.5 Hz, 1H), 7.68 (d, J=8.4 Hz, 1H), 7.45 (t, J=7.8 Hz, 1H), 6.31 (d, J=8.2 Hz, 1H), 6.09 (d, J=7.8 Hz, 1H), 5.35-5.24 (m, 1H), 4.93 (br d, J=7.2 Hz, 1H), 4.80-4.72 (m, 1H), 4.66 (br d, J=5.9 Hz, 1H), 4.53-4.45 (m, 1H), 4.40 (d, J=6.0 Hz, 2H), 4.10-3.93 (m, 4H), 3.87 (dd, J=6.2, 7.9 Hz, 2H), 3.55 (br t, J=4.8 Hz, 4H), 3.07 (br s, 1H), 2.96 (s, 3H), 2.88-2.76 (m, 1H), 2.71-2.61 (m, 4H), 2.60-2.49 (m, 1H).

### Example 57

### (S)-2-((4-(6-(5-cyanoisoindolin-2-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(5-cyanoisoindolin-2-yl)pyridin-2-yl)piperazin-1-yl)methyl)-1-(oxetan-2-ylmethyl)-1H-benzo[d]imidazole-6-carboxylic acid (Compound 57)

1H NMR (400 MHz, DMSO-d6) δ 8.33 (s, 1H), 7.98 (dd, J=1.2, 8.5 Hz, 1H), 7.74 (s, 1H), 7.66 (t, J=7.7 Hz, 2H), 7.54 (d, J=7.9 Hz, 1H), 7.38 (t, J=7.9 Hz, 1H), 6.09 (d, J=7.9 Hz, 1H), 5.94 (d, J=7.9 Hz, 1H), 5.35-5.23 (m, 1H), 4.95-4.91 (m, 1H), 4.78 (br d, J=5.3 Hz, 5H), 4.63-4.60 (m, 1H), 4.53-4.42 (m, 1H), 4.08-4.03 (m, 1H), 3.97-3.91 (m, 1H), 3.57 (br t, J=4.6 Hz, 4H), 2.87-2.75 (m, 1H), 2.69-2.62 (m, 4H), 2.58-2.49 (m, 1H).

### Example 58

### (S)-2-((4-(6-((5-chloropyridin-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((5-chloropyridin-2-yl)methoxy)pyridin-2-yl)piperazin-1-yl)methyl)-1-(oxetan-2-ylmethyl)-1H-benzo[d]imidazole-6-carboxylic acid (Compound 58)

1H NMR (400 MHz, MeOD-d4) δ8.47 (d, J=2.2 Hz, 1H), 8.30 (d, J=0.9 Hz, 1H), 7.97 (dd, J=1.4, 8.5 Hz, 1H), 7.81 (dd, J=2.5, 8.5 Hz, 1H), 7.65 (d, J=8.6 Hz, 1H), 7.49-7.42 (m, 2H), 6.27 (d, J=8.2 Hz, 1H), 6.18 (d, J=7.8 Hz, 1H), 5.36 (s, 2H), 5.26 (dd, J=2.6, 7.3 Hz, 1H), 4.92-4.86 (m, 1H), 4.75-4.68 (m, 1H), 4.63 (dt, J=6.0, 7.9 Hz, 1H), 4.46 (td, J=6.0, 9.2 Hz, 1H), 4.04-3.97 (m, 1H), 3.93-3.86 (m, 1H), 3.43 (t, J=4.9 Hz, 4H), 2.78 (dtd, J=6.2, 8.1, 11.3 Hz, 1H), 2.61-2.45 (m, 5H).

### Example 59

### (S)-2-((4-(6-(6-carbamoyl-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(6-carbamoyl-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid (Compound 59)

1H NMR (400 MHz, MeOD-d4) δ 8.35 (s, 1H), 8.02-7.95 (m, 1H), 7.72-7.64 (m, 2H), 7.42-7.26 (m, 2H), 6.17 (br d, J=8.6 Hz, 1H), 6.08-6.08 (m, 1H), 5.34-5.22 (m, 1H), 4.95-4.88 (m, 1H), 4.78-4.60 (m, 3H), 4.53-4.42 (m, 1H), 4.11-4.01 (m, 1H), 3.98-3.89 (m, 1H), 3.87-3.78 (m, 2H), 3.53 (br t, J=4.4 Hz, 4H), 2.96 (br d, J=5.4 Hz, 2H), 2.87-2.74 (m, 1H), 2.73-2.58 (m, 4H), 2.58-2.45 (m, 1H).

### Example 60

### (S)-2-((4-(6-(6-cyano-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(6-cyano-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid (Compound 60)

1H NMR (400 MHz, MeOD-d4) δ 8.34 (d, J=1.0 Hz, 1H), 7.98 (dd, J=1.4, 8.4 Hz, 1H), 7.68 (d, J=8.6 Hz, 1H), 7.55-7.47 (m, 2H), 7.37 (td, J=4.0, 7.9 Hz, 2H), 6.17 (d, J=8.0 Hz, 1H), 6.09 (d, J=8.0 Hz, 1H), 5.28 (br dd, J=2.2, 7.3 Hz, 1H), 4.96-4.86 (m, 1H), 4.78-4.68 (m, 3H), 4.68-4.60 (m, 1H), 4.47 (td, J=5.8, 9.1 Hz, 1H), 4.10-4.01 (m, 1H), 3.97-3.89 (m, 1H), 3.82 (t, J=5.8 Hz, 2H), 3.53 (br t, J=4.8 Hz, 4H), 2.95 (t, J=5.8 Hz, 2H), 2.87-2.75 (m, 1H), 2.71-2.58 (m, 4H), 2.57-2.47 (m, 1H).

### Example 61

### (S)-2-((4-(6-((3-cyanooxetan-3-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((3-cyanooxetan-3-yl)methoxy)pyridin-2-yl)piperazin-1-yl)methyl)-1-(oxetan-2-ylmethyl)-1H-benzo[d]imidazole-6-carboxylic acid (Compound 61)

1H NMR (400 MHz, MeOD-d4) δ 8.34 (s, 1H), 7.98 (dd, J=1.3, 8.5 Hz, 1H), 7.68 (d, J=8.4 Hz, 1H), 7.46 (t, J=8.0 Hz, 1H), 6.33 (d, J=8.0 Hz, 1H), 6.12 (d, J=7.8 Hz, 1H), 5.27 (dq, J=2.4, 7.2 Hz, 1H), 4.92 (d, J=6.6 Hz, 2H), 4.86 (br s, 1H), 4.73 (s, 3H), 4.68 (d, J=6.6 Hz, 1H), 4.66-4.60 (m, 1H), 4.63 (s, 1H), 4.46 (br d, J=9.2 Hz, 1H), 4.04 (s, 1H), 3.96 (s, 1H), 3.56 (br t, J=4.8 Hz, 4H), 2.87-2.74 (m, 1H), 2.71-2.59 (m, 4H), 2.57-2.45 (m, 1H)

### Example 62

### (S)-2-((4-(6-((5-cyanopyridin-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((5-cyanopyridin-2-yl)methoxy)pyridin-2-yl)piperazin-1-yl)methyl)-1-(oxetan-2-ylmethyl)-1H-benzo[d]imidazole-6-carboxylic acid (Compound 62)

1H NMR (400 MHz, MeOD-d4) δ 8.84 (d, J=1.2 Hz, 1H), 8.33 (s, 1H), 8.12 (dd, J=2.0, 8.2 Hz, 1H), 7.97 (dd, J=1.4, 8.5 Hz, 1H), 7.67 (d, J=8.4 Hz, 1H), 7.59 (d, J=8.2 Hz, 1H), 7.46 (t, J=7.8 Hz, 1H), 6.31-6.17 (m, 2H), 5.45 (s, 2H), 5.30-5.20 (m, 1H), 4.89 (br d, J=7.2 Hz, 1H), 4.75-4.68 (m, 1H), 4.66-4.59 (m, 1H), 4.46 (td, J=6.0, 9.1 Hz, 1H), 4.04-3.96 (m, 1H), 3.93-3.84 (m, 1H), 3.39 (br t, J=4.8 Hz, 4H), 2.85-2.73 (m, 1H), 2.59-2.46 (m, 5H).

### Example 63

### (S)-2-((4-(6-((4-cyanobenzyl)(methyl)amino)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-((4-cyanobenzyl)(methyl)amino)pyridin-2-yl)piperazin-1-yl)methyl)-1-(oxetan-2-ylmethyl)-1H-benzo[d]imidazole-6-carboxylic acid (Compound 63)

1H NMR (400 MHz, CDCl3-d) δ 8.22 (s, 1H), 8.03 (d, J=8.6 Hz, 1H), 7.79 (d, J=8.6 Hz, 1H), 7.57 (d, J=8.2 Hz, 2H), 7.33 (t, J=7.2 Hz, 3H), 5.99 (d, J=8.2 Hz, 1H), 5.91 (d, J=8.2 Hz, 1H), 5.24 (br d, J=3.3 Hz, 1H), 4.85-4.79 (m, 2H), 4.74-4.61 (m, 3H), 4.45-4.37 (m, 1H), 4.00 (s, 2H), 3.44 (br s, 4H), 3.01 (s, 3H), 2.73 (br d, J=6.4 Hz, 1H), 2.61 (br t, J=4.7 Hz, 4H), 2.48 (br d, J=9.0 Hz, 1H).

### Example 64

### (S)-2-((4-(6-((4-carbamoylbenzyl)(methyl)amino)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-((4-carbamoylbenzyl)(methyl)amino)pyridin-2-yl)piperazin-1-yl)methyl)-1-(oxetan-2-ylmethyl)-1H-benzo[d]imidazole-6-carboxylic acid (Compound 64)

1H NMR (400 MHz, DMSO-d6) δ 10.07 (s, 2H), 8.26 (s, 1H), 7.93 (d, J=8.2 Hz, 2H), 7.80 (d, J=7.2 Hz, 1H), 7.63 (d, J=8.6 Hz, 1H), 7.58-7.49 (m, 1H), 7.49-7.35 (m, 3H), 6.54 (d, J=8.3 Hz, 1H), 6.52-6.51 (m, 1H), 5.15-5.07 (m, 1H), 4.83-4.75 (m, 1H), 4.65 (br d, J=12.9 Hz, 1H), 4.53-4.44 (m, 1H), 4.42-4.30 (m, 1H), 3.98 (br d, J=13.6 Hz, 1H), 3.85-3.78 (m, 1H), 3.75-3.70 (m, 2H), 3.55-3.47 (m, 5H), 2.71 (br d, J=11.1 Hz, 2H), 2.32 (br s, 2H), 2.28 (s, 3H).

### Example 65

### (S)-2-((4-(6-(benzo[b]thiophen-2-ylmethoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-(benzo[b]thiophen-2-ylmethoxy)pyridin-2-yl)piperazin-1-yl)methyl)-1-(oxetan-2-ylmethyl)-1H-benzo[d]imidazole-6-carboxylic acid (Compound 65)

1H NMR (400 MHz, MeOD-d4) δ 8.32 (s, 1H), 7.97 (dd, J=1.4, 8.5 Hz, 1H), 7.81-7.76 (m, 1H), 7.72 (dd, J=2.1, 6.7 Hz, 1H), 7.67 (d, J=8.4 Hz, 1H), 7.44 (t, J=7.9 Hz, 1H), 7.32 (s, 1H), 7.29 (ddd, J=1.6, 5.5, 7.4 Hz, 2H), 6.29 (d, J=8.1 Hz, 1H), 6.10 (d, J=7.8 Hz, 1H), 5.57 (s, 2H), 5.31-5.23 (m, 1H), 4.93-4.87 (m, 1H), 4.77-4.69 (m, 1H), 4.67-4.59 (m, 1H), 4.46 (td, J=5.9, 9.1 Hz, 1H), 4.07-3.99 (m, 1H), 3.96-3.89 (m, 1H), 3.57 (br t, J=4.7 Hz, 4H), 2.85-2.73 (m, 1H), 2.69-2.46 (m, 5H).

### Example 66

### (S)-2-((4-(6-(8-cyano-1,3,4,5-tetrahydro-2H-benzo[c]azepin-2-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(8-cyano-1,3,4,5-tetrahydro-2H-benzo[c]azepin-2-yl)pyridin-2-yl)piperazin-1-yl)methyl)-1-(oxetan-2-ylmethyl)-1H-benzo[d]imidazole-6-carboxylic acid (Compound 66)

1H NMR (400 MHz, MeOD-d4) δ 8.33 (d, J=0.9 Hz, 1H), 7.97 (dd, J=1.4, 8.5 Hz, 1H), 7.75-7.64 (m, 2H), 7.42 (dd, J=1.7, 7.7 Hz, 1H), 7.31-7.18 (m, 2H), 6.01 (d, J=8.1 Hz, 1H), 5.93 (d, J=8.1 Hz, 1H), 5.31-5.21 (m, 1H), 4.97-4.87 (m, 3H), 4.78-4.70 (m, 3H), 4.67-4.60 (m, 1H), 4.48 (td, J=5.9, 9.1 Hz, 1H), 4.07 (d, J=13.7 Hz, 1H), 3.97-3.88 (m, 3H), 3.57-3.44 (m, 4H), 3.13-3.04 (m, 2H), 2.87-2.75 (m, 1H), 2.71-2.47 (m, 5H), 1.83 (br s, 2H).

### Example 67

### (S)-2-((4-(6-(benzo[d]oxazol-2-ylmethoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-(benzo[d]oxazol-2-ylmethoxy)pyridin-2-yl)piperazin-1-yl)methyl)-1-(oxetan-2-ylmethyl)-1H-benzo[d]imidazole-6-carboxylic acid (Compound 67)

1H NMR (400 MHz, MeOD-d4) δ 8.32 (s, 1H), 7.98 (dd, J=1.5, 8.4 Hz, 1H), 7.67 (d, J=8.4 Hz, 1H), 7.64-7.60 (m, 1H), 7.58-7.53 (m, 1H), 7.46 (t, J=8.0 Hz, 1H), 7.36-7.29 (m, 2H), 6.27 (d, J=8.1 Hz, 1H), 6.20 (d, J=7.8 Hz, 1H), 5.49 (s, 2H), 5.22 (br d, J=4.9 Hz, 1H), 4.87-4.84 (m, 1H), 4.67 (dd, J=2.8, 15.5 Hz, 1H), 4.64-4.58 (m, 1H), 4.44 (td, J=5.9, 9.2 Hz, 1H), 3.96-3.89 (m, 1H), 3.86-3.77 (m, 1H), 3.36 (br t, J=4.9 Hz, 4H), 2.81-2.69 (m, 1H), 2.55-2.34 (m, 5H).

### Example 68

### (S)-2-((4-(6-(benzofuran-2-ylmethoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-(benzofuran-2-ylmethoxy)pyridin-2-yl)piperazin-1-yl)methyl)-1-(oxetan-2-ylmethyl)-1H-benzo[d]imidazole-6-carboxylic acid (Compound 68)

1H NMR (400 MHz, MeOD-d4) δ 8.35 (s, 1H), 7.98 (d, J=8.4 Hz, 1H), 7.69 (d, J=8.6 Hz, 1H), 7.54 (d, J=7.6 Hz, 1H), 7.48-7.41 (m, 2H), 7.30-7.22 (m, 1H), 7.22-7.15 (m, 1H), 6.78 (s, 1H), 6.30 (d, J=8.1 Hz, 1H), 6.12 (d, J=7.9 Hz, 1H), 5.40 (s, 2H), 5.32-5.22 (m, 1H), 4.95-4.88 (m, 1H), 4.79-4.71 (m, 1H), 4.68-4.59 (m, 1H), 4.47 (td, J=5.8, 9.1 Hz, 1H), 4.09-4.01 (m, 1H), 3.97-3.88 (m, 1H), 3.56 (br t, J=4.7 Hz, 4H), 2.86-2.74 (m, 1H), 2.71-2.58 (m, 4H), 2.57-2.47 (m, 1H).

### Example 69

### (S)-2-((4-(6-(benzo[d]thiazol-2-ylmethoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-(benzo[d]thiazol-2-ylmethoxy)pyridin-2-yl)piperazin-1-yl)methyl)-1-(oxetan-2-ylmethyl)-1H-benzo[d]imidazole-6-carboxylic acid (Compound 69)

1H NMR (400 MHz, MeOD-d4) δ 8.31 (d, J=0.7 Hz, 1H), 8.02-7.88 (m, 3H), 7.66 (d, J=8.4 Hz, 1H), 7.53-7.35 (m, 3H), 6.31 (d, J=8.2 Hz, 1H), 6.21 (d, J=7.8 Hz, 1H), 5.70 (s, 2H), 5.23 (dq, J=2.5, 7.2 Hz, 1H), 4.87 (br d, J=7.2 Hz, 1H), 4.69 (dd, J=2.6, 15.3 Hz, 1H), 4.65-4.57 (m, 1H), 4.44 (td, J=5.9, 9.2 Hz, 1H), 4.01-3.92 (m, 1H), 3.88-3.81 (m, 1H), 3.46 (br t, J=4.8 Hz, 4H), 2.82-2.70 (m, 1H), 2.57-2.43 (m, 5H).

### Example 70

### (S)-2-((4-(6-(naphthalen-2-ylmethoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-(naphthalen-2-ylmethoxy)pyridin-2-yl)piperazin-1-yl)methyl)-1-(oxetan-2-ylmethyl)-1H-benzo[d]imidazole-6-carboxylic acid (Compound 70)

1H NMR (400 MHz, MeOD-d4) δ 8.34 (s, 1H), 7.98 (dd, J=1.0, 8.5 Hz, 1H), 7.88-7.77 (m, 4H), 7.68 (d, J=8.6 Hz, 1H), 7.54-7.39 (m, 4H), 6.26 (d, J=8.1 Hz, 1H), 6.19-6.12 (m, 1H), 5.45 (s, 2H), 5.29-5.19 (m, 1H), 4.85-4.81 (m, 1H), 4.74-4.57 (m, 2H), 4.48-4.39 (m, 1H), 4.05-3.97 (m, 1H), 3.90 (s, 1H), 3.49 (br t, J=4.6 Hz, 4H), 2.83-2.70 (m, 1H), 2.64-2.43 (m, 5H).

### Example 71

### (S)-2-((4-(6-((1-methyl-1H-benzo[d]imidazol-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((1-methyl-1H-benzo[d]imidazol-2-yl)methoxy)pyridin-2-yl)piperazin-1-yl)methyl)-1-(oxetan-2-ylmethyl)-1H-benzo[d]imidazole-6-carboxylic acid (Compound 71)

1H NMR (400 MHz, MeOD-d4) δ 8.34 (d, J=0.9 Hz, 1H), 7.98 (dd, J=1.5, 8.6 Hz, 1H), 7.73-7.64 (m, 1H), 7.60 (d, J=7.9 Hz, 1H), 7.52-7.42 (m, 2H), 7.33-7.26 (m, 1H), 7.25-7.19 (m, 1H), 6.31 (d, J=7.9 Hz, 1H), 6.17 (d, J=7.7 Hz, 1H), 5.58 (s, 2H), 5.25 (dq, J=2.3, 7.2 Hz, 1H), 4.93-4.88 (m, 1H), 4.77-4.68 (m, 1H), 4.63 (dt, J=6.0, 7.8 Hz, 1H), 4.51-4.41 (m, 1H), 4.04-3.97 (m, 1H), 3.91 (s, 1H), 3.87 (s, 3H), 3.49 (br t, J=4.7 Hz, 4H), 2.85-2.72 (m, 1H), 2.63-2.45 (m, 5H).

### Example 72

### (S)-2-((4-(6-((1-methyl-1H-benzo[d]imidazol-6-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((1-methyl-1H-benzo[d]imidazol-6-yl)methoxy)pyridin-2-yl)piperazin-1-yl)methyl)-1-(oxetan-2-ylmethyl)-1H-benzo[d]imidazole-6-carboxylic acid (Compound 72)

1H NMR (400 MHz, CDCl3-d) δ 8.23 (s, 1H), 8.06 (dd, J=1.4, 8.5 Hz, 1H), 7.94 (s, 1H), 7.80 (t, J=8.7 Hz, 2H), 7.49 (s, 1H), 7.42 (t, J=7.6 Hz, 1H), 7.35 (d, J=8.0 Hz, 1H), 6.18 (dd, J=4.2, 7.9 Hz, 2H), 5.47 (s, 2H), 5.24 (br dd, J=3.0, 5.6 Hz, 1H), 4.78-4.60 (m, 3H), 4.40 (td, J=6.1, 9.1 Hz, 1H), 4.08-4.00 (m, 2H), 3.84 (s, 3H), 3.57-3.47 (m, 4H), 2.78-2.65 (m, 5H), 2.52-2.41 (m, 1H).

### Example 73

### (S)-2-((4-(6-(6-cyano-8-fluoro-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(6-cyano-8-fluoro-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid (Compound 73)

1H NMR (400 MHz, MeOH-d4) δ 8.32 (s, 1H), 7.97 (d, J=9.6 Hz, 1H), 7.66 (d, J=8.4 Hz, 1H), 7.44-7.31 (m, 3H), 6.20 (d, J=7.8 Hz, 1H), 6.11 (d, J=8.4 Hz, 1H), 5.27 (br d, J=5.6 Hz, 1H), 4.96-4.91 (m, 1H), 4.71 (s, 2H), 4.68-4.57 (m, 2H), 4.52-4.42 (m, 1H), 4.10-4.00 (m, 1H), 3.96-3.88 (m, 1H), 3.83 (t, J=6.0 Hz, 2H), 3.57-3.46 (m, 4H), 2.95 (t, J=5.8 Hz, 2H), 2.79 (br s, 1H), 2.69-2.58 (m, 4H), 2.54 (br d, J=8.2 Hz, 1H).

### Example 74

### (S)-2-((4-(6-(7-cyano-1,3,4,5-tetrahydro-2H-benzo[c]azepin-2-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(7-cyano-1,3,4,5-tetrahydro-2H-benzo[c]azepin-2-yl)pyridin-2-yl)piperazin-1-yl)methyl)-1-(oxetan-2-ylmethyl)-1H-benzo[d]imidazole-6-carboxylic acid (Compound 74)

1H NMR (400 MHz, MeOH-d4) δ 8.35 (d, J=1.0 Hz, 1H), 7.98 (dd, J=1.6, 8.6 Hz, 1H), 7.68 (d, J=8.6 Hz, 1H), 7.56-7.37 (m, 3H), 7.30-7.16 (m, 1H), 6.01 (d, J=8.2 Hz, 1H), 5.93 (d, J=8.0 Hz, 1H), 5.35-5.20 (m, 1H), 4.92 (br d, J=7.2 Hz, 1H), 4.81-4.71 (m, 3H), 4.69-4.60 (m, 1H), 4.48 (td, J=6.0, 9.2 Hz, 1H), 4.13-4.00 (m, 1H), 3.99-3.88 (m, 3H), 3.50 (br t, J=4.6 Hz, 4H), 3.11-2.99 (m, 2H), 2.91-2.73 (m, 1H), 2.68-2.57 (m, 4H), 2.57-2.46 (m, 1H), 1.84 (br s, 2H).

### Example 75

### (S)-2-((4-(6-(benzo[d]oxazol-6-ylmethoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-(benzo[d]oxazol-6-ylmethoxy)pyridin-2-yl)piperazin-1-yl)methyl)-1-(oxetan-2-ylmethyl)-1H-benzo[d]imidazole-6-carboxylic acid (Compound 75)

1H NMR (400 MHz, DMSO-d6) δ 8.72 (s, 1H), 8.27 (s, 1H), 7.85-7.73 (m, 3H), 7.65 (d, J=8.4 Hz, 1H), 7.52-7.41 (m, 2H), 6.31 (d, J=8.2 Hz, 1H), 6.11 (d, J=7.8 Hz, 1H), 5.41 (s, 2H), 5.15-5.04 (m, 1H), 4.84-4.74 (m, 1H), 4.70-4.61 (m, 1H), 4.53-4.44 (m, 1H), 4.41-4.32 (m, 1H), 3.97 (d, J=13.6 Hz, 1H), 3.80 (d, J=13.6 Hz, 1H), 3.50-3.38 (m, 1H), 3.45 (br s, 4H), 2.76-2.64 (m, 1H), 2.60-2.53 (m, 4H), 2.46-2.36 (m, 1H).

### Example 76

### (S)-2-((4-(6-(6-cyano-3,4-dihydroquinolin-1(2H)-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(6-cyano-3,4-dihydroquinolin-1(2H)-yl)pyridin-2-yl)piperazin-1-yl)methyl)-1-(oxetan-2-ylmethyl)-1H-benzo[d]imidazole-6-carboxylic acid (Compound 76)

1H NMR (400 MHz, MeOH-d4) δ 8.34 (d, J=1.0 Hz, 1H), 7.98 (dd, J=1.6, 8.6 Hz, 1H), 7.68 (d, J=8.4 Hz, 1H), 7.49 (t, J=8.0 Hz, 1H), 7.38 (s, 1H), 7.31-7.20 (m, 2H), 6.51 (d, J=7.8 Hz, 1H), 6.45 (d, J=8.4 Hz, 1H), 5.36-5.19 (m, 1H), 4.92 (br s, 1H), 4.79-4.70 (m, 1H), 4.69-4.59 (m, 1H), 4.47 (td, J=5.8, 9.2 Hz, 1H), 4.10-3.88 (m, 2H), 3.86-3.77 (m, 2H), 3.61-3.45 (m, 4H), 2.88-2.73 (m, 3H), 2.71-2.58 (m, 4H), 2.57-2.46 (m, 1H), 2.00 (quin, J=6.0 Hz, 2H).

### Example 77

### (S)-2-((4-(6-(7-cyano-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(7-cyano-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid (Compound 77)

1H NMR (400 MHz, MeOH-d4) δ 8.34 (br d, J=1.0 Hz, 1H), 7.98 (dd, J=1.6, 8.4 Hz, 1H), 7.68 (d, J=8.6 Hz, 1H), 7.59 (s, 1H), 7.49 (br d, J=7.8 Hz, 1H), 7.41-7.34 (m, 1H), 7.32 (d, J=7.8 Hz, 1H), 6.18 (d, J=8.0 Hz, 1H), 6.09 (d, J=8.0 Hz, 1H), 5.35-5.21 (m, 1H), 4.74 (br dd, J=2.6, 15.3 Hz, 1H), 4.70-4.60 (m, 2H), 4.48 (td, J=5.8, 9.0 Hz, 1H), 4.10-3.90 (m, 2H), 3.83 (br t, J=5.8 Hz, 2H), 3.53 (br t, J=4.6 Hz, 3H), 2.98 (br t, J=5.8 Hz, 2H), 2.88-2.74 (m, 1H), 2.71-2.59 (m, 3H), 2.59-2.44 (m, 1H).

### Example 78

### (S)-2-((4-(6-(6-cyano-1,3,4,5-tetrahydro-2H-benzo[c]azepin-2-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(6-cyano-1,3,4,5-tetrahydro-2H-benzo[c]azepin-2-yl)pyridin-2-yl)piperazin-1-yl)methyl)-1-(oxetan-2-ylmethyl)-1H-benzo[d]imidazole-6-carboxylic acid (Compound 78)

1H NMR (400 MHz, MeOH-d4) δ 8.39-8.32 (m, 1H), 8.02-7.95 (m, 1H), 7.70 (s, 2H), 7.58-7.42 (m, 1H), 7.36-7.20 (m, 2H), 6.02 (d, J=8.2 Hz, 2H), 5.32-5.22 (m, 1H), 4.78 (s, 2H), 4.64 (br d, J=5.6 Hz, 1H), 4.57-4.40 (m, 1H), 4.11-4.05 (m, 1H), 4.02-3.92 (m, 3H), 3.51 (br s, 5H), 2.86 (s, 2H), 2.82-2.74 (m, 2H), 2.71-2.60 (m, 4H), 2.57-2.48 (m, 1H), 1.94-1.83 (m, 2H).

### Example 79

### (S)-2-((4-(6-(6-cyano-1,2,4,5-tetrahydro-3H-benzo[d]azepin-3-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(6-cyano-1,2,4,5-tetrahydro-3H-benzo[d]azepin-3-yl)pyridin-2-yl)piperazin-1-yl)methyl)-1-(oxetan-2-ylmethyl)-1H-benzo[d]imidazole-6-carboxylic acid (Compound 79)

1H NMR (400 MHz, MeOH-d4) δ 8.35 (s, 1H), 7.98 (br d, J=8.4 Hz, 1H), 7.68 (br d, J=8.4 Hz, 1H), 7.49 (br d, J=7.6 Hz, 1H), 7.42 (br d, J=6.8 Hz, 1H), 7.34 (br t, J=7.8 Hz, 1H), 7.24 (br t, J=7.6 Hz, 1H), 6.14 (br d, J=8.4 Hz, 1H), 6.04 (br d, J=7.8 Hz, 1H), 5.28 (br d, J=6.2 Hz, 1H), 4.75 (br d, J=15.8 Hz, 2H), 4.68-4.61 (m, 1H), 4.49 (br s, 1H), 4.07 (br d, J=13.0 Hz, 1H), 3.95 (br d, J=13.8 Hz, 1H), 3.89 (br s, 2H), 3.81 (br s, 2H), 3.53 (br s, 4H), 3.27 (br s, 2H), 3.04 (br s, 2H), 2.88-2.76 (m, 2H), 2.67 (br s, 3H), 2.54 (br s, 1H).

### Example 80

### (S)-2-((4-(6-(9-cyano-1,3,4,5-tetrahydro-2H-benzo[c]azepin-2-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(9-cyano-1,3,4,5-tetrahydro-2H-benzo[c]azepin-2-yl)pyridin-2-yl)piperazin-1-yl)methyl)-1-(oxetan-2-ylmethyl)-1H-benzo[d]imidazole-6-carboxylic acid (Compound 80)

1H NMR (400 MHz, CDCl3-d) δ 8.24 (s, 1H), 8.06 (d, J=8.6 Hz, 1H), 7.82 (d, J=8.4 Hz, 1H), 7.44 (d, J=7.0 Hz, 1H), 7.34-7.28 (m, 2H), 7.22-7.15 (m, 1H), 6.14 (d, J=8.2 Hz, 1H), 5.90 (d, J=8.2 Hz, 1H), 5.24 (br d, J=6.6 Hz, 1H), 4.97 (br s, 2H), 4.81-4.73 (m, 1H), 4.72-4.59 (m, 2H), 4.42 (td, J=6.0, 9.0 Hz, 1H), 4.02 (s, 2H), 3.93 (br s, 2H), 3.58-3.41 (m, 4H), 3.02-2.92 (m, 2H), 2.80-2.70 (m, 1H), 2.66 (br t, J=4.8 Hz, 4H), 2.53-2.41 (m, 1H), 1.94 (br s, 2H).

### Example 81

### (S)-2-((4-(6-((5-carbamoylpyrimidin-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure P.

### (S)-2-((4-(6-((5-carbamoylpyrimidin-2-yl)methoxy)pyridin-2-yl)piperazin-1-yl)methyl)-1-(oxetan-2-ylmethyl)-1H-benzo[d]imidazole-6-carboxylic acid (Compound 81)

1H NMR (400 MHz, MeOH-d4) δ 9.12 (s, 2H), 8.23 (s, 1H), 7.94 (s, 1H), 7.61 (d, J=8.4 Hz, 1H), 7.44 (t, J=7.8 Hz, 1H), 6.22 (dd, J=2.8, 7.8 Hz, 2H), 5.48 (s, 2H), 5.25 (br dd, J=2.6, 7.2 Hz, 1H), 4.70 (br d, J=2.6 Hz, 1H), 4.67-4.59 (m, 3H), 4.45 (td, J=5.8, 9.0 Hz, 1H), 3.95 (s, 1H), 3.87 (s, 1H), 3.27 (br s, 3H), 2.80-2.73 (m, 1H), 2.52 (br d, J=8.6 Hz, 6H).

### Example 82

### (S)-1-(oxetan-2-ylmethyl)-2-((4-(6-(quinolin-2-ylmethoxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-(quinolin-2-ylmethoxy)pyridin-2-yl)piperazin-1-yl)methyl)-1H-benzo[d]imidazole-6-carboxylic acid (Compound 82)

1H NMR (400 MHz, CDCl3-d) δ 8.23 (s, 1H), 8.16 (d, J=8.6 Hz, 1H), 8.11-8.02 (m, 2H), 7.81 (d, J=8.4 Hz, 2H), 7.72-7.65 (m, 1H), 7.62 (d, J=8.6 Hz, 1H), 7.54-7.47 (m, 1H), 7.47-7.41 (m, 1H), 6.26 (d, J=7.8 Hz, 1H), 6.16 (d, J=8.0 Hz, 1H), 5.63 (s, 2H), 5.21 (br d, J=3.8 Hz, 1H), 4.79-4.56 (m, 3H), 4.39 (td, J=5.8, 9.0 Hz, 1H), 3.96 (s, 2H), 3.54-3.31 (m, 4H), 2.78-2.68 (m, 1H), 2.54 (br t, J=4.6 Hz, 4H), 2.49-2.40 (m, 1H).

### Example 83

### 2-((4-(6-((R)-6-cyano-1-methyl-3,4-dihydroisoquinolin-2(1H)-yl)pyridin-2-yl)piperazin-1-yl)methyl)-1-(((S)-oxetan-2-yl)methyl)-1H-benzo[d]imidazole-6-carboxylic acid

The title compound was prepared and can be prepared similarly following the procedures described by General Procedure B.

### 2-((4-(6-((R)-6-cyano-1-methyl-3,4-dihydroisoquinolin-2(1H)-yl)pyridin-2-yl)piperazin-1-yl)methyl)-1-(((S)-oxetan-2-yl)methyl)-1H-benzo[d]imidazole-6-carboxylic acid (Compound 83)

1H NMR (400 MHz, CDCl3-d) δ 8.23 (s, 1H), 8.05 (br d, J=8.4 Hz, 1H), 7.82 (d, J=8.6 Hz, 1H), 7.49-7.41 (m, 2H), 7.36 (t, J=8.0 Hz, 1H), 7.24 (s, 1H), 6.05 (br d, J=7.8 Hz, 1H), 5.98 (d, J=7.8 Hz, 1H), 5.50 (br d, J=6.8 Hz, 1H), 5.31-5.20 (m, 1H), 4.84-4.70 (m, 2H), 4.69-4.60 (m, 1H), 4.47-4.38 (m, 1H), 4.18 (td, J=4.8, 12.8 Hz, 1H), 4.10-3.98 (m, 2H), 3.54 (br s, 4H), 3.45-3.34 (m, 1H), 3.07-2.93 (m, 1H), 2.86 (td, J=3.8, 16.2 Hz, 1H), 2.81-2.56 (m, 5H), 2.55-2.41 (m, 2H), 1.48 (d, J=6.8 Hz, 3H).

### Example 84

### 2-((4-(6-((S)-6-cyano-1-methyl-3,4-dihydroisoquinolin-2(1H)-yl)pyridin-2-yl)piperazin-1-yl)methyl)-1-(((S)-oxetan-2-yl)methyl)-1H-benzo[d]imidazole-6-carboxylic acid

The title compound was prepared and can be prepared similarly following the procedures described by General Procedure B.

### 2-((4-(6-((S)-6-cyano-1-methyl-3,4-dihydroisoquinolin-2(1H)-yl)pyridin-2-yl)piperazin-1-yl)methyl)-1-(((S)-oxetan-2-yl)methyl)-1H-benzo[d]imidazole-6-carboxylic acid (Compound 84)

1H NMR (400 MHz, CDCl3-d) δ 8.25 (s, 1H), 8.07 (br d, J=8.4 Hz, 1H), 7.82 (br d, J=8.4 Hz, 1H), 7.49-7.42 (m, 2H), 7.36 (br t, J=8.0 Hz, 1H), 7.25 (d, J=8.2 Hz, 1H), 6.04 (d, J=8.2 Hz, 1H), 5.98 (d, J=8.2 Hz, 1H), 5.50 (br d, J=6.8 Hz, 1H), 5.26 (br d, J=3.2 Hz, 1H), 4.82-4.70 (m, 2H), 4.70-4.60 (m, 1H), 4.42 (td, J=5.8, 8.8 Hz, 1H), 4.18 (td, J=4.8, 13.0 Hz, 1H), 4.05 (s, 2H), 3.53 (br d, J=4.8 Hz, 4H), 3.41 (ddd, J=4.2, 9.6, 13.4 Hz, 1H), 3.06-2.95 (m, 1H), 2.86 (td, J=3.8, 16.2 Hz, 1H), 2.81-2.65 (m, 5H), 2.55-2.42 (m, 2H), 1.48 (d, J=6.8 Hz, 3H).

### Example 85

### (S)-2-((4-(6-(isoquinolin-3-ylmethoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-(isoquinolin-3-ylmethoxy)pyridin-2-yl)piperazin-1-yl)methyl)-1-(oxetan-2-ylmethyl)-1H-benzo[d]imidazole-6-carboxylic acid (Compound 85)

1H NMR (400 MHz, CDCl3-d) δ 9.31 (s, 1H), 8.22 (s, 1H), 8.04 (br d, J=8.6 Hz, 1H), 8.00 (br d, J=8.2 Hz, 1H), 7.87-7.79 (m, 3H), 7.70 (t, J=7.6 Hz, 1H), 7.64-7.53 (m, 1H), 7.46 (t, J=7.8 Hz, 1H), 6.29 (d, J=7.8 Hz, 1H), 6.19 (d, J=8.0 Hz, 1H), 5.66 (s, 2H), 5.23 (br s, 1H), 4.76-4.57 (m, 3H), 4.45-4.30 (m, 1H), 4.01 (s, 2H), 3.51 (br d, J=5.0 Hz, 4H), 2.77-2.69 (m, 1H), 2.65 (br s, 4H), 2.49-2.39 (m, 1H).

### Example 86

### (S)-2-((4-(6-((1-methyl-1H-pyrazol-4-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((1-methyl-1H-pyrazol-4-yl)methoxy)pyridin-2-yl)piperazin-1-yl)methyl)-1-(oxetan-2-ylmethyl)-1H-benzo[d]imidazole-6-carboxylic acid (Compound 86)

1H NMR (400 MHz, MeOH-d4) δ 8.37 (s, 1H), 8.00 (dd, J=1.4, 8.4 Hz, 1H), 7.70 (d, J=8.4 Hz, 1H), 7.63 (s, 1H), 7.51 (s, 1H), 7.43 (t, J=8.0 Hz, 1H), 6.28 (d, J=7.8 Hz, 1H), 6.06 (d, J=7.8 Hz, 1H), 5.36-5.24 (m, 1H), 5.20 (s, 2H), 4.97-4.91 (m, 1H), 4.81-4.73 (m, 1H), 4.71-4.62 (m, 1H), 4.49 (td, J=5.8, 9.0 Hz, 1H), 4.12-4.04 (m, 1H), 4.01-3.93 (m, 1H), 3.87 (s, 3H), 3.58 (br t, J=4.8 Hz, 4H), 2.88-2.75 (m, 1H), 2.74-2.63 (m, 4H), 2.55 (br s, 1H).

### Example 87

(R)-4-(4-(6-((4-cyano-2-fluorobenzyl)oxy)pyridin-2-yl)piperidin-1-yl)-1,2,3,4-tetrahydrobenzo[4,5]imidazo[1,2-a]pyridine-8-carboxylic acid

The title compound was prepared and can be prepared similarly following the procedures described by General Procedure F.

(R)-4-(4-(6-((4-cyano-2-fluorobenzyl)oxy)pyridin-2-yl)piperidin-1-yl)-1,2,3,4-tetrahydrobenzo[4,5]imidazo[1,2-a]pyridine-8-carboxylic acid (Compound 87). 1HNMR (400 MHz, MeOH-d4) δ 8.18 (d, J=1.0 Hz, 1H), 7.98 (dd, J=1.6, 8.6 Hz, 1H), 7.69 (d, J=8.0 Hz, 2H), 7.65-7.51 (m, 3H), 6.87 (d, J=7.0 Hz, 1H), 6.69 (d, J=8.0 Hz, 1H), 5.53 (s, 2H), 4.37-4.23 (m, 2H), 4.19-4.08 (m, 1H), 3.15 (br d, J=12.0 Hz, 1H), 3.05 (br d, J=10.8 Hz, 1H), 2.97-2.84 (m, 1H), 2.81-2.62 (m, 2H), 2.42 (br dd, J=6.2, 11.0 Hz, 1H), 2.36-2.26 (m, 1H), 2.23-2.06 (m, 2H), 2.05-1.83 (m, 4H).

### Example 88

### (S)-2-((4-(6-(cyclobutylmethoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-(cyclobutylmethoxy)pyridin-2-yl)piperazin-1-yl)methyl)-1-(oxetan-2-ylmethyl)-1H-benzo[d]imidazole-6-carboxylic acid (Compound 88)

1H NMR (400 MHz, MeOH-d4) δ 8.34 (s, 1H), 7.96 (d, J=1.2 Hz, 1H), 7.67 (d, J=8.4 Hz, 1H), 7.40 (t, J=7.8 Hz, 1H), 6.23 (d, J=8.0 Hz, 1H), 6.02 (d, J=7.8 Hz, 1H), 5.34-5.19 (m, 1H), 4.87 (br d, J=7.2 Hz, 1H), 4.77-4.70 (m, 1H), 4.68-4.59 (m, 1H), 4.50-4.42 (m, 1H), 4.16 (d, J=6.8 Hz, 2H), 4.09-4.01 (m, 1H), 3.97-3.89 (m, 1H), 3.52 (br t, J=4.8 Hz, 4H), 2.86-2.68 (m, 2H), 2.67-2.58 (m, 4H), 2.57-2.45 (m, 1H), 2.14-2.02 (m, 2H), 2.01-1.78 (m, 4H).

### Example 89

### (S)-4-(4-(6-((4-cyano-2-fluorobenzyl)oxy)pyridin-2-yl)piperidin-1-yl)-1,2,3,4-tetrahydrobenzo[4,5]imidazo[1,2-a]pyridine-8-carboxylic acid

The title compound was prepared and can be prepared similarly following the procedures described by General Procedure F.

### (S)-4-(4-(6-((4-cyano-2-fluorobenzyl)oxy)pyridin-2-yl)piperidin-1-yl)-1,2,3,4-tetrahydrobenzo[4,5]imidazo[1,2-a]pyridine-8-carboxylic acid (Compound 89)

1HNMR (400 MHz, MeOH-d4) δ 8.18 (d, J=1.0 Hz, 1H), 8.01-7.95 (m, 1H), 7.69 (d, J=8.0 Hz, 2H), 7.64-7.52 (m, 3H), 6.86 (d, J=7.4 Hz, 1H), 6.69 (d, J=8.0 Hz, 1H), 5.53 (s, 2H), 4.36-4.24 (m, 2H), 4.19-4.08 (m, 1H), 3.15 (br d, J=10.8 Hz, 1H), 3.05 (br d, J=10.8 Hz, 1H), 2.96-2.85 (m, 1H), 2.81-2.63 (m, 2H), 2.41 (br dd, J=6.0, 10.8 Hz, 1H), 2.35-2.25 (m, 1H), 2.23-2.07 (m, 2H), 2.05-1.83 (m, 4H).

### Example 90

### (R)-6-(4-(6-((4-cyano-2-fluorobenzyl)oxy)pyridin-2-yl)piperidin-1-yl)-7,8,9,10-tetrahydro-6H-benzo[4,5]imidazo[1,2-a]azepine-2-carboxylic acid

The title compound was prepared and can be prepared similarly following the procedures described by General Procedure F.

### (R)-6-(4-(6-((4-cyano-2-fluorobenzyl)oxy)pyridin-2-yl)piperidin-1-yl)-7,8,9,10-tetrahydro-6H-benzo[4,5]imidazo[1,2-a]azepine-2-carboxylic acid (Compound 90)

1H NMR (400 MHz, MeOD-d4) δ 8.22 (s, 1H), 7.95 (dd, J=1.2, 8.4 Hz, 1H), 7.70-7.49 (m, 5H), 6.82 (d, J=7.4 Hz, 1H), 6.67 (d, J=8.4 Hz, 1H), 5.56-5.46 (m, 2H), 4.66-4.46 (m, 3H), 3.79 (br s, 1H), 2.63 (br s, 1H), 2.40 (br s, 2H), 2.33-2.02 (m, 4H), 1.88 (br d, J=5.4 Hz, 3H), 1.74 (br s, 4H).

### Example 91

### (S)-6-(4-(6-((4-cyano-2-fluorobenzyl)oxy)pyridin-2-yl)piperidin-1-yl)-7,8,9,10-tetrahydro-6H-benzo[4,5]imidazo[1,2-a]azepine-2-carboxylic acid

The title compound was prepared and can be prepared similarly following the procedures described by General Procedure F.

### (S)-6-(4-(6-((4-cyano-2-fluorobenzyl)oxy)pyridin-2-yl)piperidin-1-yl)-7,8,9,10-tetrahydro-6H-benzo[4,5]imidazo[1,2-a]azepine-2-carboxylic acid (Compound 91)

1H NMR (400 MHz, MeOD-d4) δ 8.23 (s, 1H), 7.96 (dd, J=1.4, 8.4 Hz, 1H), 7.71-7.51 (m, 5H), 6.84 (d, J=7.2 Hz, 1H), 6.69 (d, J=8.4 Hz, 1H), 5.57-5.47 (m, 2H), 4.70-4.47 (m, 3H), 3.82 (br d, J=5.4 Hz, 1H), 2.73-2.56 (m, 1H), 2.51-2.34 (m, 2H), 2.34-2.03 (m, 4H), 1.97-1.81 (m, 3H), 1.80-1.48 (m, 4H).

### Example 92

### (S)-2-((4-(3-((4-cyano-2-fluorobenzyl)oxy)-1H-pyrazol-1-yl)piperidin-1-yl)methyl)-1-(oxetan-2-ylmethyl)-1H-benzo[d]imidazole-6-carboxylic acid

The title compound was prepared and can be prepared similarly following the procedures described by General Procedure U.

### (S)-2-((4-(3-((4-cyano-2-fluorobenzyl)oxy)-1H-pyrazol-1-yl)piperidin-1-yl)methyl)-1-(oxetan-2-ylmethyl)-1H-benzo[d]imidazole-6-carboxylic acid (Compound 92)

1H NMR (400 MHz, MeOD-d4) δ 8.33 (s, 1H), 7.97 (dd, J=1.6, 8.4 Hz, 1H), 7.72 (t, J=7.6 Hz, 1H), 7.67 (d, J=8.4 Hz, 1H), 7.59 (s, 1H), 7.57 (s, 1H), 7.46 (d, J=2.4 Hz, 1H), 5.72 (d, J=2.4 Hz, 1H), 5.28 (s, 2H), 5.25 (br d, J=2.0 Hz, 1H), 4.91 (br d, J=7.2 Hz, 1H), 4.73 (dd, J=2.6, 15.6 Hz, 1H), 4.69-4.57 (m, 1H), 4.47 (td, J=6.0, 9.2 Hz, 1H), 4.09-3.88 (m, 3H), 3.05 (br d, J=12.2 Hz, 1H), 2.94 (br d, J=11.2 Hz, 1H), 2.88-2.76 (m, 1H), 2.60-2.46 (m, 1H), 2.45-2.25 (m, 2H), 2.10-1.93 (m, 4H).

### Example 93

### (S)-5-(4-(6-(benzyloxy)pyridin-2-yl)piperidin-1-yl)-1,2,4,5-tetrahydrobenzo[4,5]imidazo[1,2-d][1,4]oxazepine-9-carboxylic acid

The title compound was prepared and can be prepared similarly following the procedures described by General Procedure G.

### (S)-5-(4-(6-(benzyloxy)pyridin-2-yl)piperidin-1-yl)-1,2,4,5-tetrahydrobenzo[4,5]imidazo[1,2-d][1,4]oxazepine-9-carboxylic acid (Compound 93)

1H NMR (400 MHz, CDCl3-d) δ 8.15 (s, 1H), 8.06 (dd, J=1.4, 8.6 Hz, 1H), 7.82 (d, J=8.4 Hz, 1H), 7.53-7.45 (m, 3H), 7.39-7.33 (m, 2H), 7.31 (d, J=7.2 Hz, 1H), 6.72 (d, J=7.2 Hz, 1H), 6.61 (d, J=8.2 Hz, 1H), 5.38 (s, 2H), 5.04-4.96 (m, 1H), 4.53 (dd, J=4.2, 13.8 Hz, 1H), 4.33 (br d, J=13.2 Hz, 2H), 3.75-3.69 (m, 2H), 3.68-3.53 (m, 2H), 2.73 (br d, J=11.6 Hz, 1H), 2.69-2.61 (m, 1H), 2.38-2.21 (m, 2H), 2.01 (br s, 2H), 1.87-1.73 (m, 2H).

### Example 94

### (R)-5-(4-(6-(benzyloxy)pyridin-2-yl)piperidin-1-yl)-1,2,4,5-tetrahydrobenzo[4,5]imidazo[1,2-d][1,4]oxazepine-9-carboxylic acid

The title compound was prepared and can be prepared similarly following the procedures described by General Procedure G.

### (R)-5-(4-(6-(benzyloxy)pyridin-2-yl)piperidin-1-yl)-1,2,4,5-tetrahydrobenzo[4,5]imidazo[1,2-d][1,4]oxazepine-9-carboxylic acid (Compound 94)

1H NMR (400 MHz, CDCl3-d) δ 8.07 (s, 1H), 7.98 (dd, J=1.2, 8.4 Hz, 1H), 7.74 (d, J=8.6 Hz, 1H), 7.43-7.36 (m, 3H), 7.31-7.21 (m, 3H), 6.64 (d, J=7.2 Hz, 1H), 6.53 (d, J=8.2 Hz, 1H), 5.30 (s, 2H), 4.97-4.88 (m, 1H), 4.45 (dd, J=3.8, 13.4 Hz, 1H), 4.25 (d, J=12.6 Hz, 2H), 3.65 (br d, J=13.6 Hz, 2H), 3.59-3.45 (m, 2H), 2.67 (td, J=1.4, 7.0 Hz, 1H), 2.63-2.53 (m, 1H), 2.34-2.08 (m, 2H), 2.04-1.84 (m, 2H), 1.79-1.65 (m, 2H).

### Example 95

### (1R,5R)-5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylic acid

The title compound was prepared and can be prepared similarly following the procedures described by General Procedure H.

### (1R,5R)-5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylic acid (Compound 95)

1H NMR (400 MHz, CDCl3-d) δ 8.19 (s, 1H), 8.05 (br d, J=8.4 Hz, 1H), 7.83 (d, J=8.4 Hz, 1H), 7.66 (t, J=6.8 Hz, 1H), 7.59-7.49 (m, 1H), 7.49-7.42 (m, 1H), 7.39 (d, J=9.4 Hz, 1H), 6.81 (d, J=7.2 Hz, 1H), 6.66 (d, J=8.2 Hz, 1H), 5.53 (s, 2H), 4.86 (br s, 1H), 4.38 (br d, J=10.4 Hz, 1H), 4.24 (br d, J=7.2 Hz, 1H), 4.16-4.10 (m, 1H), 4.07-4.01 (m, 1H), 3.90 (br dd, J=8.6, 12.2 Hz, 1H), 3.51-3.43 (m, 1H), 3.14-2.94 (m, 2H), 2.83-2.64 (m, 2H), 2.03-1.86 (m, 4H), 1.64 (br d, J=7.0 Hz, 3H).

### Example 96

### (1R,5S)-5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylic acid

The title compound was prepared and can be prepared similarly following the procedures described by General Procedure H.

### (1R,5S)-5-(4-(6-((4-cyano-2-fluorobenzyl)oxy)pyridin-2-yl)piperidin-1-yl)-1-methyl-1,2,4,5-tetrahydrobenzo[4,5]imidazo[1,2-d][1,4]oxazepine-9-carboxylic acid (Compound 96)

1H NMR (400 MHz, CDCl3-d) δ 8.24-8.14 (m, 1H), 8.08 (br d, J=8.2 Hz, 1H), 7.83 (br d, J=8.2 Hz, 1H), 7.61 (br t, J=7.4 Hz, 1H), 7.52 (br t, J=7.8 Hz, 1H), 7.42 (br d, J=8.0 Hz, 1H), 7.36 (br d, J=8.6 Hz, 1H), 6.75 (br d, J=7.4 Hz, 1H), 6.69-6.58 (m, 1H), 5.49 (s, 2H), 4.63 (br d, J=10.4 Hz, 2H), 4.29 (br d, J=13.0 Hz, 1H), 3.84 (br d, J=13.2 Hz, 1H), 3.79-3.73 (m, 1H), 3.72-3.55 (m, 2H), 2.69-2.53 (m, 2H), 2.39-2.13 (m, 2H), 1.99 (br d, J=7.0 Hz, 3H), 1.77 (br d, J=12.4 Hz, 2H), 1.71-1.44 (m, 2H).

### Example 97

### 2-(((1S,6R)-6-(6-(benzyloxy)pyridin-2-yl)-3-azabicyclo[4.1.0]heptan-3-yl)methyl)-1-(((S)-oxetan-2-yl)methyl)-1H-benzo[d]imidazole-6-carboxylic acid

The title compound was prepared and can be prepared similarly following the procedures described by General Procedure J.

### 2-(((1S,6R)-6-(6-(benzyloxy)pyridin-2-yl)-3-azabicyclo[4.1.0]heptan-3-yl)methyl)-1-(((S)-oxetan-2-yl)methyl)-1H-benzo[d]imidazole-6-carboxylic acid (Compound 97)

1H NMR (400 MHz, MeOD-d4) δ 8.27 (s, 1H), 7.96 (dd, J=1.4, 8.6 Hz, 1H), 7.65 (d, J=8.4 Hz, 1H), 7.53 (t, J=7.8 Hz, 1H), 7.42-7.35 (m, 2H), 7.31 (t, J=7.6 Hz, 3H), 6.87 (d, J=7.4 Hz, 1H), 6.55 (d, J=8.2 Hz, 1H), 5.32 (d, J=2.2 Hz, 2H), 5.26-5.18 (m, 1H), 4.86-4.81 (m, 1H), 4.71 (s, 1H), 4.63-4.53 (m, 1H), 4.46-4.36 (m, 1H), 3.92 (q, J=13.8 Hz, 2H), 2.97 (dd, J=6.4, 11.4 Hz, 1H), 2.82-2.67 (m, 2H), 2.64-2.54 (m, 1H), 2.44 (s, 3H), 2.13-2.04 (m, 1H), 1.86-1.76 (m, 1H), 1.24 (s, 1H), 0.95 (dd, J=3.8, 5.8 Hz, 1H).

### Example 98

### 2-(((1S,6R)-6-(6-((4-chloro-2-fluorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid

The title compound was prepared and can be prepared similarly following the procedures described by General Procedure K.

### 2-(((1S,6R)-6-(6-((4-chloro-2-fluorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (Compound 98)

1H NMR (400 MHz, MeOD-d4) δ 8.28 (s, 1H), 7.97 (br d, J=8.6 Hz, 1H), 7.65 (br d, J=8.4 Hz, 1H), 7.55 (t, J=7.8 Hz, 1H), 7.44 (t, J=8.0 Hz, 1H), 7.24-7.10 (m, 2H), 6.90 (d, J=7.4 Hz, 1H), 6.56 (d, J=8.0 Hz, 1H), 5.36 (s, 2H), 5.28-5.17 (m, 1H), 4.86 (br s, 1H), 4.75-4.64 (m, 1H), 4.59 (br d, J=6.4 Hz, 1H), 4.41 (br d, J=9.2 Hz, 1H), 3.92 (q, J=13.8 Hz, 2H), 3.03-2.90 (m, 1H), 2.82-2.68 (m, 2H), 2.58 (br dd, J=6.4, 13.1 Hz, 1H), 2.52-2.38 (m, 3H), 2.15-2.02 (m, 1H), 1.79 (br d, J=7.6 Hz, 1H), 1.20 (br dd, J=3.4, 8.8 Hz, 1H), 1.00-0.90 (m, 1H).

### Example 99

### (S)-1-(oxetan-2-ylmethyl)-2-((4-(3-phenethoxy-1H-pyrazol-1-yl)piperidin-1-yl)methyl)-1H-benzo[d]imidazole-6-carboxylic acid

The title compound was prepared and can be prepared similarly following the procedures described by General Procedure U.

### (S)-1-(oxetan-2-ylmethyl)-2-((4-(3-phenethoxy-1H-pyrazol-1-yl)piperidin-1-yl)methyl)-1H-benzo[d]imidazole-6-carboxylic acid (Compound 99)

1H NMR (400 MHz, CDCl3-d) δ 8.27-8.11 (m, 1H), 8.06 (br d, J=8.4 Hz, 1H), 7.82 (d, J=8.6 Hz, 1H), 7.34-7.17 (m, 6H), 5.61 (d, J=1.8 Hz, 1H), 5.20 (br d, J=5.4 Hz, 1H), 4.81-4.57 (m, 3H), 4.47-4.33 (m, 1H), 4.33-4.24 (m, 2H), 4.04 (s, 2H), 4.01-3.88 (m, 1H), 3.07 (br t, J=7.2 Hz, 3H), 3.00 (br s, 1H), 2.91-2.67 (m, 1H), 2.49-2.28 (m, 3H), 2.06 (br d, J=9.8 Hz, 4H)

### Example 100

### (S)-2-((4-(3-(4-cyano-2-fluorobenzyl)-3H-imidazo[4,5-b]pyridin-5-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure AA.

### (S)-2-((4-(3-(4-cyano-2-fluorobenzyl)-3H-imidazo[4,5-b]pyridin-5-yl)piperazin-1-yl)methyl)-1-(oxetan-2-ylmethyl)-1H-benzo[d]imidazole-6-carboxylic acid (Compound 100)

1H NMR (400 MHz, MeOD-d4) δ 8.33 (d, J=0.8 Hz, 1H), 8.12 (s, 1H), 7.97 (dd, J=1.6, 8.6 Hz, 1H), 7.79 (d, J=9.0 Hz, 1H), 7.68 (d, J=8.6 Hz, 1H), 7.59 (dd, J=1.4, 9.8 Hz, 1H), 7.54-7.42 (m, 2H), 6.81 (d, J=9.0 Hz, 1H), 5.52 (s, 2H), 5.36-5.21 (m, 1H), 4.96-4.90 (m, 1H), 4.79-4.69 (m, 1H), 4.68-4.59 (m, 1H), 4.47 (td, J=6.0, 9.2 Hz, 1H), 4.09-3.88 (m, 2H), 3.58 (br t, J=4.8 Hz, 4H), 2.85-2.74 (m, 1H), 2.71-2.58 (m, 4H), 2.58-2.47 (m, 1H).

### Example 101

### (S)-2-((4-(3-(benzo[d]thiazol-2-ylmethoxy)-1H-pyrazol-1-yl)piperidin-1-yl)methyl)-1-(oxetan-2-ylmethyl)-1H-benzo[d]imidazole-6-carboxylic acid

The title compound was prepared and can be prepared similarly following the procedures described by General Procedure U.

### (S)-2-((4-(3-(benzo[d]thiazol-2-ylmethoxy)-1H-pyrazol-1-yl)piperidin-1-yl)methyl)-1-(oxetan-2-ylmethyl)-1H-benzo[d]imidazole-6-carboxylic acid (Compound 101)

1H NMR (400 MHz, CHLOROFORM-d) δ 8.21 (s, 1H), 8.09-8.01 (m, 2H), 7.89 (d, J=7.8 Hz, 1H), 7.81 (d, J=8.4 Hz, 1H), 7.52-7.46 (m, 1H), 7.43-7.36 (m, 1H), 7.24 (d, J=2.2 Hz, 1H), 5.75 (d, J=2.2 Hz, 1H), 5.61-5.60 (m, 1H), 5.62 (s, 1H), 5.27-5.18 (m, 1H), 4.82-4.59 (m, 3H), 4.40 (td, J=5.8, 9.0 Hz, 1H), 4.08-3.91 (m, 3H), 3.09-2.97 (m, 2H), 2.82-2.71 (m, 1H), 2.52-2.28 (m, 3H), 2.18-1.96 (m, 4H).

### Example 102 (General Procedure GG)

### (S)-2-((4-(4-chloro-3-((4-cyano-2-fluorobenzyl)oxy)-1H-pyrazol-1-yl)piperidin-1-yl)methyl)-1-(oxetan-2-ylmethyl)-1H-benzo[d]imidazole-6-carboxylic acid

The title compound was prepared according to Scheme 17. This General Procedure GG exemplifies Scheme 17 and provides particular synthetic details as applied to the title compound.

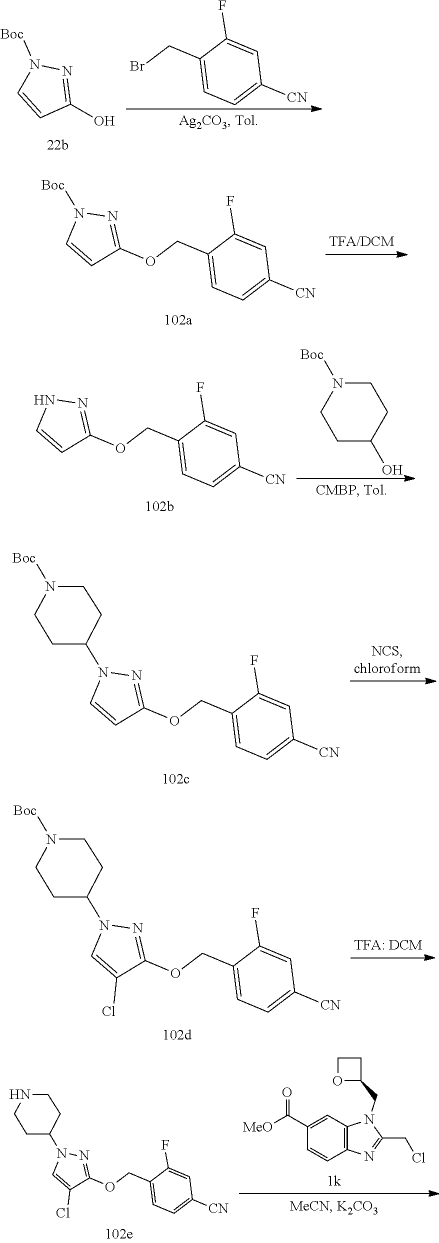

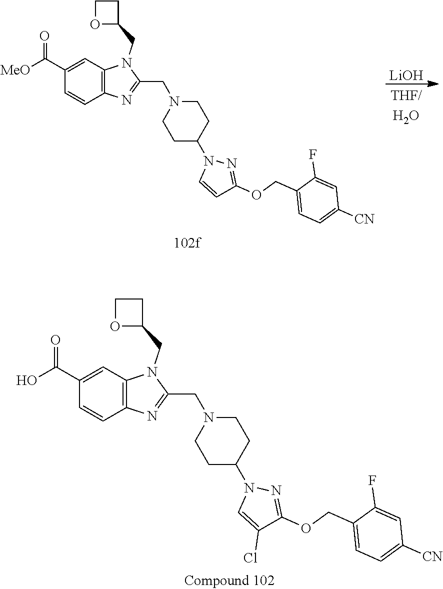 [SMILES: COC(=O)c1ccc2nc(CN3CCC(n4ccc(OCc5ccc(C#N)cc5F)n4)CC3)n(C[C@@H]3CCO3)c2c1 ; "LiOH" ; N#Cc1ccc(COc2nn(C3CCN(Cc4nc5ccc(C(=O)O)cc5n4C[C@@H]4CCO4)CC3)cc2Cl)c(F)c1]

### tert-butyl 3-((4-cyano-2-fluorobenzyl)oxy)-1H-pyrazole-1-carboxylate (102a)

To the solution of tert-butyl 3-hydroxy-1H-pyrazole-1-carboxylate (22b, 5 g, 27.15 mmol) and 4-(bromomethyl)-3-fluoro-benzonitrile (6.10 g, 28.50 mmol) in Tol. (120 mL) was added Ag2CO3 (14.97 g, 54.29 mmol, 2.46 mL). The mixture was stirred at 100° C. for 3 hours. TLC indicated 23b was consumed, and one new spot was detected. The reaction mixture was diluted with Ethyl acetate (30 mL). The mixture was filtered and concentrated under reduced pressure. The residue was purified by column chromatography (SiO2, Petroleum ether:Ethyl acetate=5:1 to 3:1) to give 102a as a white solid. 1H NMR (400 MHz, CDCl3) δ 7.88 (d, J=3.0 Hz, 1H), 7.69 (t, J=7.6 Hz, 1H), 7.49 (dd, J=1.6, 8.0 Hz, 1H), 7.38 (dd, J=1.6, 9.2 Hz, 1H), 5.94 (d, J=3.0 Hz, 1H), 5.46 (s, 2H), 1.63 (s, 9H).

### 4-(((1H-pyrazol-3-yl)oxy)methyl)-3-fluorobenzonitrile (102b)

A solution of tert-butyl 3-[(4-cyano-2-fluoro-phenyl)methoxy]pyrazole-1-carboxylate (5 g, 15.76 mmol) in TFA (5 mL) and DCM (50 mL) was stirred at 20° C. for 2 hours. LCMS showed 102a was consumed completely and desired mass was detected. The reaction mixture was concentrated under reduced pressure. The residue was diluted with Ethyl acetate (100 mL) and washed with NaHCO3 (aq) (30 mL), dried over Na2SO4, filtered and concentrated under reduced pressure to give 102b as a white solid. 1H NMR (400 MHz, CDCl3-d) δ 7.69 (t, J=7.4 Hz, 1H), 7.48 (d, J=8.2 Hz, 1H), 7.42-7.36 (m, 2H), 5.83 (d, J=2.4 Hz, 1H), 5.38 (s, 2H).

### Tert-butyl 4-(3-((4-cyano-2-fluorobenzyl)oxy)-1H-pyrazol-1-yl)piperidine-1-carboxylate (102c)

To the solution of 3-fluoro-4-(1H-pyrazol-3-yloxymethyl)benzonitrile (102b, 1g, 4.60 mmol) and tert-butyl 4-hydroxypiperidine-1-carboxylate (1.85 g, 9.21 mmol) in Tol. (30 mL) at 20° C. Then 2-(tributyl-λ5-phosphanylidene) acetonitrile (5.56 g, 23.02 mmol) was added. The mixture was stirred at 100° C. for 4 hours. LC-MS showed 102b was consumed completely and desired mass was detected. The reaction mixture was concentrated under reduced pressure. The residue was diluted with Ethyl acetate (30 mL) and washed with H2O (10 mL), dried over Na2SO4, filtered and concentrated under reduced pressure. The residue was purified by column chromatography (SiO2, Petroleum ether:Ethyl acetate=5:1 to 3:1) to give 102c as a white solid. 1H NMR (400 MHz, CDCl3-d) δ 7.70 (t, J=7.4 Hz, 1H), 7.48 (d, J=7.8 Hz, 1H), 7.37 (dd, J=1.0, 9.4 Hz, 1H), 7.23 (s, 1H), 5.70 (d, J=2.4 Hz, 1H), 5.31 (s, 2H), 4.34-4.15 (m, 2H), 4.15-4.00 (m, 1H), 2.88 (br t, J=12.4 Hz, 2H), 2.12-2.03 (m, 2H), 1.95-1.74 (m, 2H), 1.48 (s, 9H).

### Tert-butyl 4-(4-chloro-3-((4-cyano-2-fluorobenzyl)oxy)-1H-pyrazol-1-yl)piperidine-1-carboxylate (102d)

To a mixture of tert-butyl 4-(3-((4-cyano-2-fluorobenzyl)oxy)-1H-pyrazol-1-yl)piperidine-1-carboxylate (102c, 100 mg, 249.72 umol) in CHCl3 (2 mL) was added NCS (50.02 mg, 374.58 umol) under N2. The mixture was stirred at 60° C. for 2 hours. TLC indicated the starting material was consumed completely and one new spot was formed. The reaction mixture was concentrated under reduced pressure to give 102d as colorless oil. 1H NMR (400 MHz, MeOD-d4) δ 7.75-7.67 (m, 1H), 7.65 (d, J=4.8 Hz, 1H), 7.67-7.54 (m, 2H), 5.36 (s, 2H), 4.83 (s, 16H), 4.16-4.09 (m, 3H), 3.31 (td, J=1.6, 3.2 Hz, 2H), 2.92 (br s, 2H), 1.98 (br d, J=9.6 Hz, 2H), 1.79 (dt, J=4.6, 12.2 Hz, 2H), 1.49-1.46 (m, 9H).

### 4-(((4-chloro-1-(piperidin-4-yl)-1H-pyrazol-3-yl)oxy)methyl)-3-fluorobenzonitrile (102e)

To a mixture of tert-butyl 4-(4-chloro-3-((4-cyano-2-fluorobenzyl)oxy)-1H-pyrazol-1-yl)piperidine-1-carboxylate (102d, 80 mg, 183.95 umol) in DCM (5 mL) was added TFA (0.5 mL) under N2. The mixture was stirred at 20° C. for 2 hours. TLC (Petroleum ether:Ethyl acetate=1:1, Rf=0.0) indicated the starting material was consumed completely and one new spot was formed. The reaction mixture was concentrated under reduced pressure to give 102e as a white solid. MS mass calculated for [M+H]+ (C16H16ClFN4O) requires m/z 335.0, LCMS found m/z 335.0.

### (S)-methyl 2-((4-(3-((4-cyano-2-fluorobenzyl)oxy)-1H-pyrazol-1-yl)piperidin-1-yl)methyl)-1-(oxetan-2-ylmethyl)-1H-benzo[d]imidazole-6-carboxylate (102f)

To a mixture of 4-(((4-chloro-1-(piperidin-4-yl)-1H-pyrazol-3-yl)oxy)methyl)-3-fluorobenzonitrile (102e, 52.82 mg, 179.22 umol) and 4-[[4-chloro-1-(4-piperidyl)pyrazol-3-yl]oxymethyl]-3-fluoro-benzonitrile (1k, 60 mg, 179.22 umol) in CH3CN (2 mL) was added K2CO3 (74.31 mg, 537.67 umol) at 20° C. under N2. The mixture was stirred at 50° C. for 16 hours. LCMS showed the starting material was consumed completely and desired mass was detected. TLC (Petroleum ether:Ethyl acetate=0:1, Rf=0.4) indicated the starting material was consumed completely and one new spot was formed. The reaction mixture was poured into water (20 mL) and extracted with ethyl acetate (30 mL*2). The combined organic phase was washed with brine (20 mL*2), dried with anhydrous Na2SO4, filtered and concentrated in vacuum. The residue was purified by prep-TLC (SiO2, Petroleum ether/Ethyl acetate=0/1) to give 102f as a light yellow solid. 1H NMR (400 MHz, CDCl3-d) δ 8.15 (d, J=0.8 Hz, 1H), 7.98 (dd, J=1.4, 8.6 Hz, 1H), 7.79-7.68 (m, 2H), 7.52-7.46 (m, 1H), 7.41-7.35 (m, 1H), 7.27 (s, 1H), 5.38 (s, 2H), 5.26-5.17 (m, 1H), 4.74-4.54 (m, 3H), 4.38 (td, J=6.0, 9.0 Hz, 1H), 4.00 (br d, J=5.6 Hz, 2H), 3.96 (s, 3H), 3.94-3.83 (m, 1H), 3.04-2.95 (m, 2H), 2.79-2.70 (m, 1H), 2.54-2.37 (m, 1H), 2.37-2.25 (m, 2H), 2.05 (br s, 2H), 2.01-1.87 (m, 2H).

### (S)-2-((4-(4-chloro-3-((4-cyano-2-fluorobenzyl)oxy)-1H-pyrazol-1-yl)piperidin-1-yl)methyl)-1-(oxetan-2-ylmethyl)-1H-benzo[d]imidazole-6-carboxylic acid (Compound 102)

To a mixture of (S)-methyl 2-((4-(3-((4-cyano-2-fluorobenzyl)oxy)-1H-pyrazol-1-yl)piperidin-1-yl)methyl)-1-(oxetan-2-ylmethyl)-1H-benzo[d]imidazole-6-carboxylate (102f, 84 mg, 141.64 umol) in THF (2.8 mL) was added LiOH·H2O (5.94 mg, 141.64 umol) in H2O (1.2 mL) under N2. The mixture was stirred at 20° C. for 16 hours. LCMS showed the starting material was consumed and desired mass was detected. The mixture was quenched by addition of citric (10%) to adjust pH=6-7, and the reaction mixture were concentrated under reduced pressure. The residue was purified by prep-HPLC (column: Waters Xbridge BEH C18 100*30 mm*10 um; mobile phase: [water (10 mM NH4HCO3)-ACN]; B %: 15%-45%, 8 min) to give Compound 102 as a white solid. 1H NMR (400 MHz, METHANOL-d4) δ 8.34-8.32 (m, 1H), 7.99-7.94 (m, 1H), 7.71 (br t, J=7.2 Hz, 2H), 7.58 (t, J=4.4 Hz, 3H), 5.36 (s, 2H), 5.27-5.22 (m, 1H), 4.89 (br d, J=7.2 Hz, 1H), 4.74 (d, J=2.2 Hz, 1H), 4.63 (s, 1H), 4.49-4.42 (m, 1H), 4.05-3.89 (m, 3H), 3.05-2.89 (m, 2H), 2.80 (br d, J=8.8 Hz, 1H), 2.56-2.47 (m, 1H), 2.39-2.27 (m, 2H), 1.99 (br d, J=7.2 Hz, 4H).

### Example 103

### (S)-2-((4-(6-(benzo[d]oxazol-5-ylmethoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-(benzo[d]oxazol-5-ylmethoxy)pyridin-2-yl)piperazin-1-yl)methyl)-1-(oxetan-2-ylmethyl)-1H-benzo[d]imidazole-6-carboxylic acid (Compound 103)

1H NMR (400 MHz, CHLOROFORM-d) δ 8.24 (br s, 1H), 8.12-8.03 (m, 2H), 7.86 (s, 1H), 7.82 (d, J=7.8 Hz, 1H), 7.58-27.53 (m, 1H), 7.48 (d, J=8.6 Hz, 1H), 7.42 (t, J=7.4 Hz, 1H), 6.18 (d, J=7.8 Hz, 2H), 5.44 (s, 2H), 5.25 (br s, 1H), 4.82-4.61 (m, 3H), 4.42 (td, J=5.8, 9.0 Hz, 1H), 4.04 (br s, 2H), 3.65-3.42 (m, 4H), 2.81-2.60 (m, 5H), 2.56-2.43 (m, 1H).

### Example 104

### 2-(((1R,6S)-6-(6-(benzo[d]thiazol-2-ylmethoxy)pyridin-2-yl)-3-azabicyclo[4.1.0]heptan-3-yl)methyl)-1-(((S)-oxetan-2-yl)methyl)-1H-benzo[d]imidazole-6-carboxylic acid

### and

### 2-(((1S,6R)-6-(6-(benzo[d]thiazol-2-ylmethoxy)pyridin-2-yl)-3-azabicyclo[4.1.0]heptan-3-yl)methyl)-1-(((S)-oxetan-2-yl)methyl)-1H-benzo[d]imidazole-6-carboxylic acid

The title compounds were prepared and can be prepared similarly following the procedures described by General Procedure K.

### 2-(((1R,6S)-6-(6-(benzo[d]thiazol-2-ylmethoxy)pyridin-2-yl)-3-azabicyclo[4.1.0]heptan-3-yl)methyl)-1-(((S)-oxetan-2-yl)methyl)-1H-benzo[d]imidazole-6-carboxylic acid (104-P1)

1H NMR (400 MHz, CHLOROFORM-d) δ 8.24 (s, 1H) 8.03 (t, J=8.4 Hz, 2H) 7.86 (d, J=8.0 Hz, 1H) 7.80 (d, J=8.6 Hz, 1H) 7.54 (t, J=7.8 Hz, 1H) 7.43-7.50 (m, 1H) 7.33-7.40 (m, 1H) 6.87 (d, J=7.2 Hz, 1H) 6.64-6.69 (m, 1H) 5.77 (s, 2H) 5.18 (br s, 1H) 4.73 (br s, 1H) 4.58-4.70 (m, 2H) 4.38-4.44 (m, 1H) 3.92 (br s, 2H) 2.84 (br s, 2H) 2.66-2.76 (m, 1H) 2.57 (br s, 1H) 2.45 (br d, J=9.2 Hz, 2H) 2.29-2.40 (m, 1H) 2.01 (br d, J=2.6 Hz, 1H) 1.86 (br s, 1H) 1.26 (br s, 1H) 0.88-1.03 (m, 1H).

### 2-(((1S,6R)-6-(6-(benzo[d]thiazol-2-ylmethoxy)pyridin-2-yl)-3-azabicyclo[4.1.0]heptan-3-yl)methyl)-1-(((S)-oxetan-2-yl)methyl)-1H-benzo[d]imidazole-6-carboxylic acid (Compound 104-P2)

1H NMR (400 MHz, CHLOROFORM-d) δ 8.21 (s, 1H) 8.03 (t, J=8.6 Hz, 2H) 7.86 (d, J=8.0 Hz, 1H) 7.80 (d, J=8.6 Hz, 1H) 7.54 (t, J=7.8 Hz, 1H) 7.47 (t, J=7.2 Hz, 1H) 7.37 (t, J=7.2 Hz, 1H) 6.86 (d, J=7.4 Hz, 1H) 6.67 (d, J=8.0 Hz, 1H) 5.73-5.80 (m, 2H) 5.19 (br d, J=4.2 Hz, 1H) 4.70-4.80 (m, 1H) 4.56-4.67 (m, 2H) 4.36 (dt, J=8.8, 6.0 Hz, 1H) 3.93 (br s, 2H) 2.91 (br s, 1H) 2.80 (br d, J=10.2 Hz, 1H) 2.63-2.76 (m, 1H) 2.49-2.61 (m, 1H) 2.41 (br s, 3H) 1.98-2.15 (m, 1H) 1.78-1.88 (m, 1H) 1.19-1.32 (m, 1H) 0.93 (br s, 1H).

When a mixture of stereoisomers is separated by HPLC, it is to be appreciated that the resultant individual stereoisomers or mixtures will be arbitrarily assigned. In the examples described herein, when the mixture of stereoisomers is separated by HPLC, it is to be appreciated that an eluting enantiomer or an enantiomer of a resulting compound prepared from the eluting enantiomer is labeled “P1” and another eluting enantiomer or an enantiomer of a resulting compound prepared from the another eluting enantiomer is labeled “P2”. In this example, the resulting compound is Compound 104. The absolute configuration of the enantiomers, e.g., Compounds 104-P1 & 104-P2 each associated with the corresponding 1H NMR data, may be obtained by known methods.

### Example 105

### (S)-2-((4-(3-(6-cyano-3,4-dihydroisoquinolin-2(1H)-yl)-1H-pyrazol-1-yl)piperidin-1-yl)methyl)-1-(oxetan-2-ylmethyl)-1H-benzo[d]imidazole-6-carboxylic acid

The title compound was prepared and can be prepared similarly following the procedures described by General Procedure Y.

### (S)-2-((4-(3-(6-cyano-3,4-dihydroisoquinolin-2(1H)-yl)-1H-pyrazol-1-yl)piperidin-1-yl)methyl)-1-(oxetan-2-ylmethyl)-1H-benzo[d]imidazole-6-carboxylic acid (Compound 105)

1H NMR (400 MHz, CHLOROFORM-d) δ 8.19 (s, 1H), 8.03 (br d, J=8.2 Hz, 1H), 7.80 (d, J=8.4 Hz, 1H), 7.44 (br s, 2H), 7.26-7.20 (m, 2H), 5.74 (s, 1H), 5.29-5.17 (m, 1H), 4.80-4.59 (m, 3H), 4.49-4.35 (m, 3H), 4.01 (br s, 3H), 3.53 (br t, J=5.2 Hz, 2H), 3.10-2.95 (m, 4H), 2.84-2.70 (m, 1H), 2.54-2.29 (m, 4H), 2.23-2.09 (m, 2H), 2.06-1.93 (m, 3H).

### Example 106

### (S)-2-((4-(3-(7-cyano-1,2,4,5-tetrahydro-3H-benzo[d]azepin-3-yl)-1H-pyrazol-1-yl)piperidin-1-yl)methyl)-1-(oxetan-2-ylmethyl)-1H-benzo[d]imidazole-6-carboxylic acid

The title compound was prepared and can be prepared similarly following the procedures described by General Procedure Y.

### (S)-2-((4-(3-(7-cyano-1,2,4,5-tetrahydro-3H-benzo[d]azepin-3-yl)-1H-pyrazol-1-yl)piperidin-1-yl)methyl)-1-(oxetan-2-ylmethyl)-1H-benzo[d]imidazole-6-carboxylic acid (Compound 106)

1H NMR (400 MHz, CHLOROFORM-d) δ 8.19 (s, 1H) 8.06 (d, J=8.0 Hz, 1H) 7.82 (d, J=8.4 Hz, 1H) 7.36-7.42 (m, 2H) 7.19 (d, J=7.4 Hz, 1H) 5.64 (d, J=2.2 Hz, 1H) 5.23 (br dd, J=6.0, 2.6 Hz, 1H) 4.60-4.78 (m, 3H) 4.40 (dt, J=9.0, 6.0 Hz, 1 H) 3.96-4.10 (m, 3H) 3.48-3.63 (m, 4H) 2.92-3.15 (m, 6H) 2.62-2.88 (m, 2H) 2.33-2.61 (m, 4H) 2.17 (br d, J=13.0 Hz, 2H) 1.92-2.10 (m, 2H).

### Example 107

### (S)-2-((4-(6-((4-fluorobenzo[d]thiazol-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((4-fluorobenzo[d]thiazol-2-yl)methoxy)pyridin-2-yl)piperazin-1-yl)methyl)-1-(oxetan-2-ylmethyl)-1H-benzo[d]imidazole-6-carboxylic acid (Compound 107)

11H NMR (400 MHz, CHLOROFORM-d) δ 8.23 (s, 1H), 8.05 (d, J=8.4 Hz, 1H), 7.81 (d, J=8.6 Hz, 1H), 7.63 (d, J=7.8 Hz, 1H), 7.47 (t, J=7.8 Hz, 1H), 7.34 (dt, J=4.6, 8.0 Hz, 1H), 7.21-7.12 (m, 1H), 6.23 (dd, J=8.0, 16.2 Hz, 2H), 5.77 (s, 2H), 5.28-5.19 (m, 1H), 5.37-5.15 (m, 1H), 4.83-4.72 (m, 1H), 4.71-4.59 (m, 2H), 4.46-4.38 (m, 1H), 4.02 (br s, 2H), 3.51 (br s, 4H), 2.83-2.70 (m, 1H), 2.64 (br s, 4H), 2.54-2.40 (m, 1H).

### Example 108 (General Procedure HH)

### (S)-2-((4-(3-((4-cyano-2-fluorobenzyl)oxy)-4-fluoro-1H-pyrazol-1-yl)piperidin-1-yl)methyl)-1-(oxetan-2-ylmethyl)-1H-benzo[d]imidazole-6-carboxylic acid

The title compound was prepared according to Scheme 17. This General Procedure HH exemplifies Scheme 17 and provides particular synthetic details as applied to the title compound.

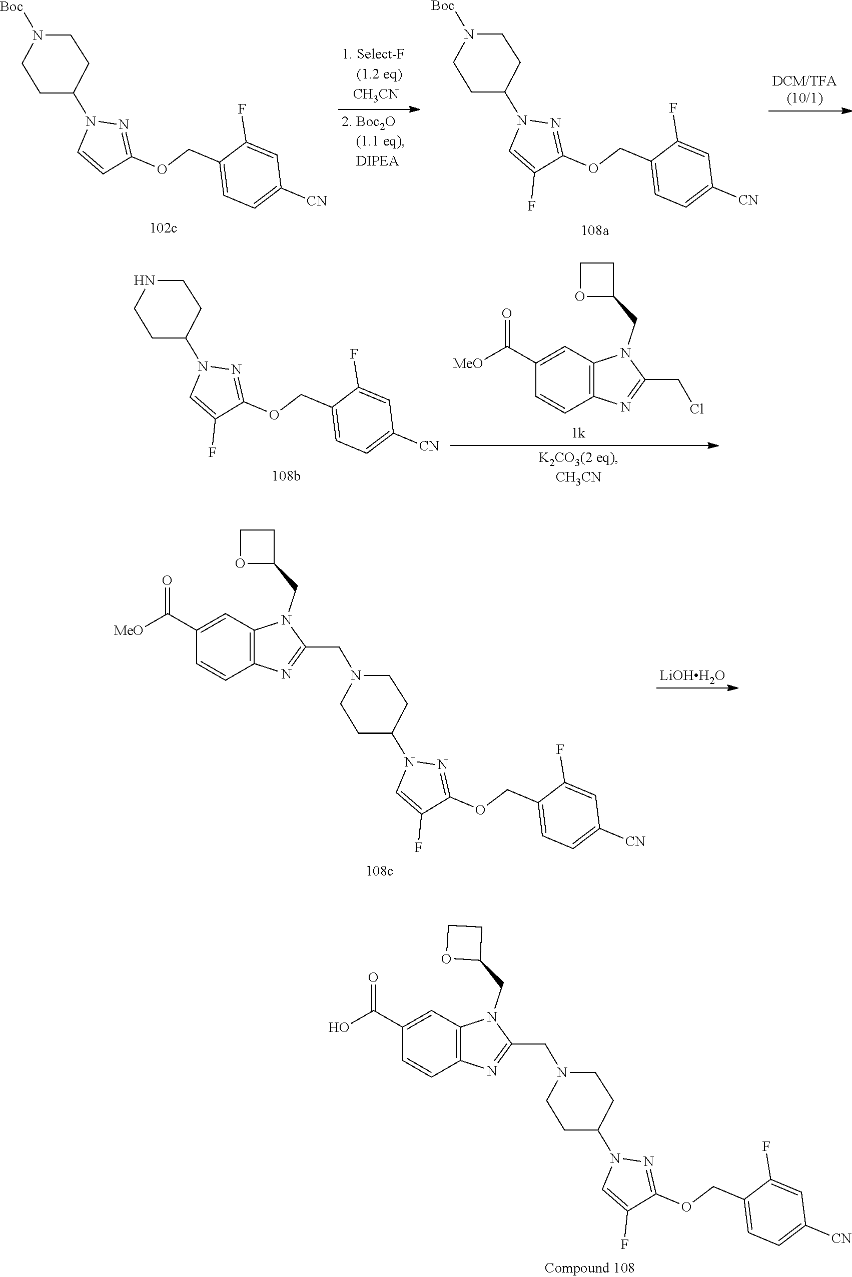

### Tert-butyl 4-(3-((4-cyano-2-fluorobenzyl)oxy)-4-fluoro-1H-pyrazol-1-yl)piperidine-1-carboxylate (108a)

To a solution of tert-butyl 4-[3-[(4-cyano-2-fluoro-phenyl)methoxy]pyrazol-1-yl]piperidine-1-carboxylate (108c, 75 mg, 187.29 umol) in CH3CN (2 mL) was added Select F (99.52 mg, 280.94 umol) at 20° C. The mixture was stirred at 60° C. for 16 hours. LCMS showed a little reactant was remained. Then DIPEA (48.41 mg, 374.58 umol, 65.25 uL) and Boc2O (61.31 mg, 280.94 umol, 64.54 uL) was added at 20° C. The mixture was stirred at 20° C. for 2 hours. LC-MS showed 108c was consumed completely desired mass was detected. The mixture was concentrated under reduced pressure. The residue was diluted with Ethyl acetate (20 mL) and washed with NH4Cl (aq) (10 mL), dried over Na2SO4, filtered and concentrated under reduced pressure. The residue was purified by prep-TLC (SiO2, Petroleum ether/Ethyl acetate=2:1) to give 108a as a light yellow oil, checked by HNMR (ET15812-1470-P1A). 1H NMR (400 MHz, CDCl3-d) δ 7.71 (t, J=7.4 Hz, 1H), 7.49 (br d, J=7.2 Hz, 1H), 7.38 (d, J=9.2 Hz, 1H), 7.19 (d, J=4.4 Hz, 1H), 5.36 (s, 2H), 4.20 (br s, 2H), 4.00-3.89 (m, 1H), 2.86 (br t, J=11.6 Hz, 2H), 2.04 (br d, J=14.4 Hz, 2H), 1.80 (qd, J=12.2, 16.3 Hz, 2H), 1.48 (s, 9H).

### 3-fluoro-4-(((4-fluoro-1-(piperidin-4-yl)-1H-pyrazol-3-yl)oxy)methyl)benzonitrile (108b)

A solution of tert-butyl 4-[3-[(4-cyano-2-fluoro-phenyl)methoxy]-4-fluoro-pyrazol-1-yl]piperidine-1-carboxylate (108a, 60 mg, 143.39 umol) in TFA (0.2 mL) and DCM (2 mL) was stirred at 20° C. for 1 hour. LCMS showed 108a was consumed completely and desired mass was detected. The mixture was concentrated under reduced pressureto give 108b as light yellow oil. The product was used directly in next step.

### (S)-methyl 2-((4-(3-((4-cyano-2-fluorobenzyl)oxy)-4-fluoro-1H-pyrazol-1-yl)piperidin-1-yl)methyl)-1-(oxetan-2-ylmethyl)-1H-benzo[d]imidazole-6-carboxylate (108c)

To a solution of 3-fluoro-4-[[4-fluoro-1-(4-piperidyl)pyrazol-3-yl]oxymethyl]benzonitrile (108b, 46 mg, 144.51 umol) and in CH3CN (2 mL) were added K2CO3 (79.89 mg, 578.03 umol) and methyl 2-(chloromethyl)-3-[[(2S)-oxetan-2-yl]methyl]benzimidazole-5-carboxylate (1k, 42.59 mg, 144.51 umol) at 20° C. under N2. The mixture was stirred at 60° C. for 16 hours. LCMS showed 108b was consumed completely and desired mass was detected. The reaction mixture was diluted with Ethyl acetate (30 mL) and washed with H2O (10 mL), dried over Na2SO4, filtered and concentrated under reduced pressure. The residue was purified by prep-TLC (SiO2, Petroleum ether:Ethyl acetate=3:1) to give 108c as light yellow oil. 1H NMR (400 MHz, CDCl3-d) δ 8.15 (s, 1H), 8.13-8.13 (m, 1H), 7.98 (d, J=7.8 Hz, 1H), 7.75 (d, J=8.6 Hz, 1H), 7.70 (t, J=7.4 Hz, 1H), 7.47 (d, J=7.8 Hz, 1H), 7.36 (d, J=9.2 Hz, 1H), 5.39-5.34 (m, 2H), 5.26-5.17 (m, 1H), 4.75-4.66 (m, 2H), 4.66-4.59 (m, 1H), 4.37 (td, J=5.8, 9.2 Hz, 1H), 4.04-3.97 (m, 2H), 3.95 (s, 3H), 3.91-3.79 (m, 1H), 2.99 (br s, 2H), 2.81-2.69 (m, 1H), 2.51-2.39 (m, 1H), 2.38-2.24 (m, 2H), 2.05 (br s, 2H), 1.98-1.83 (m, 2H).

### (S)-2-((4-(3-((4-cyano-2-fluorobenzyl)oxy)-4-fluoro-1H-pyrazol-1-yl)piperidin-1-yl)methyl)-1-(oxetan-2-ylmethyl)-1H-benzo[d]imidazole-6-carboxylic acid (Compound 108)

To a solution of methyl 2-[[4-[3-[(4-cyano-2-fluoro-phenyl)methoxy]-4-fluoro-pyrazol-1-yl]-1-piperidyl]methyl]-3-[[(2S)-oxetan-2-yl]methyl]benzimidazole-5-carboxylate (108c, 25 mg, 43.36 umol) in THF (1.4 mL) and H2O (0.6 mL) was added LiOH·H2O (3.64 mg, 86.72 umol) at 20° C. The mixture was stirred at 20° C. for 16 hours. LCMS showed 40c was consumed completely and desired mass was detected. The mixture was adjusted to pH 6 with AcOH. Then the mixture was concentrated under reduced pressure to remove THF. The aqueous layer was extracted with i-PrOH/DCM (1/10, 20 mL). The organic layer was concentrated under reduced pressure. The residue was purified by prep-HPLC (column: Phenomenex Gemini-NX C18 75*30 mm*3 um; mobile phase: [water (10 mM NH4HCO3)-ACN]; B %: 20%-40%, 6 min) to give Compound 108 as a white solid. 1H NMR (400 MHz, CDCl3-d) δ 8.22 (s, 1H), 8.06 (d, J=7.8 Hz, 1H), 7.82 (d, J=8.6 Hz, 1H), 7.73 (t, J=7.6 Hz, 1H), 7.50 (d, J=7.6 Hz, 1H), 7.39 (d, J=9.2 Hz, 1H), 7.21 (d, J=4.8 Hz, 1H), 5.38 (s, 2H), 5.24 (br dd, J=3.0, 6.2 Hz, 1H), 4.78-4.63 (m, 3H), 4.41 (td, J=6.0, 9.2 Hz, 1H), 4.08-3.99 (m, 2H), 3.92-3.82 (m, 1H), 3.03 (br t, J=10.4 Hz, 2H), 2.83-2.68 (m, 1H), 2.53-2.42 (m, 1H), 2.40-2.29 (m, 2H), 2.06 (br s, 2H), 2.00-1.88 (m, 2H).

### Example 109

### (S)-2-((4-(6-(8-cyano-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(8-cyano-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid (Compound 109)

1H NMR (400 MHz, CHLOROFORM-d) δ 8.25 (s, 1H), 8.05 (d, J=9.4 Hz, 1H), 7.81 (d, J=8.4 Hz, 1H), 7.51 (d, J=7.6 Hz, 1H), 7.43-7.33 (m, 2H), 7.27-7.22 (m, 1H), 6.13 (d, J=8.0 Hz, 1H), 6.03 (d, J=8.2 Hz, 1H), 5.25 (br dd, J=2.6, 6.6 Hz, 1H), 4.87 (s, 2H), 4.84-4.60 (m, 3H), 4.43 (td, J=5.8, 9.0 Hz, 1H), 4.02 (s, 2H), 3.85 (t, J=5.8 Hz, 2H), 3.64-3.49 (m, 4H), 2.96 (br t, J=5.8 Hz, 2H), 2.83-2.64 (m, 5H), 2.56-2.43 (m, 1H).

### Example 110 (General Procedure II)

### (S)-2-((4-(6-((6-cyanobenzo[d]thiazol-2-yl)methoxy)pyridin-2-yl)piperazin-1-yl)methyl)-1-(oxetan-2-ylmethyl)-1H-benzo[d]imidazole-6-carboxylic acid

The title compound was prepared according to Scheme 24. This General Procedure II exemplifies Scheme 24 and provides particular synthetic details as applied to the title compound.

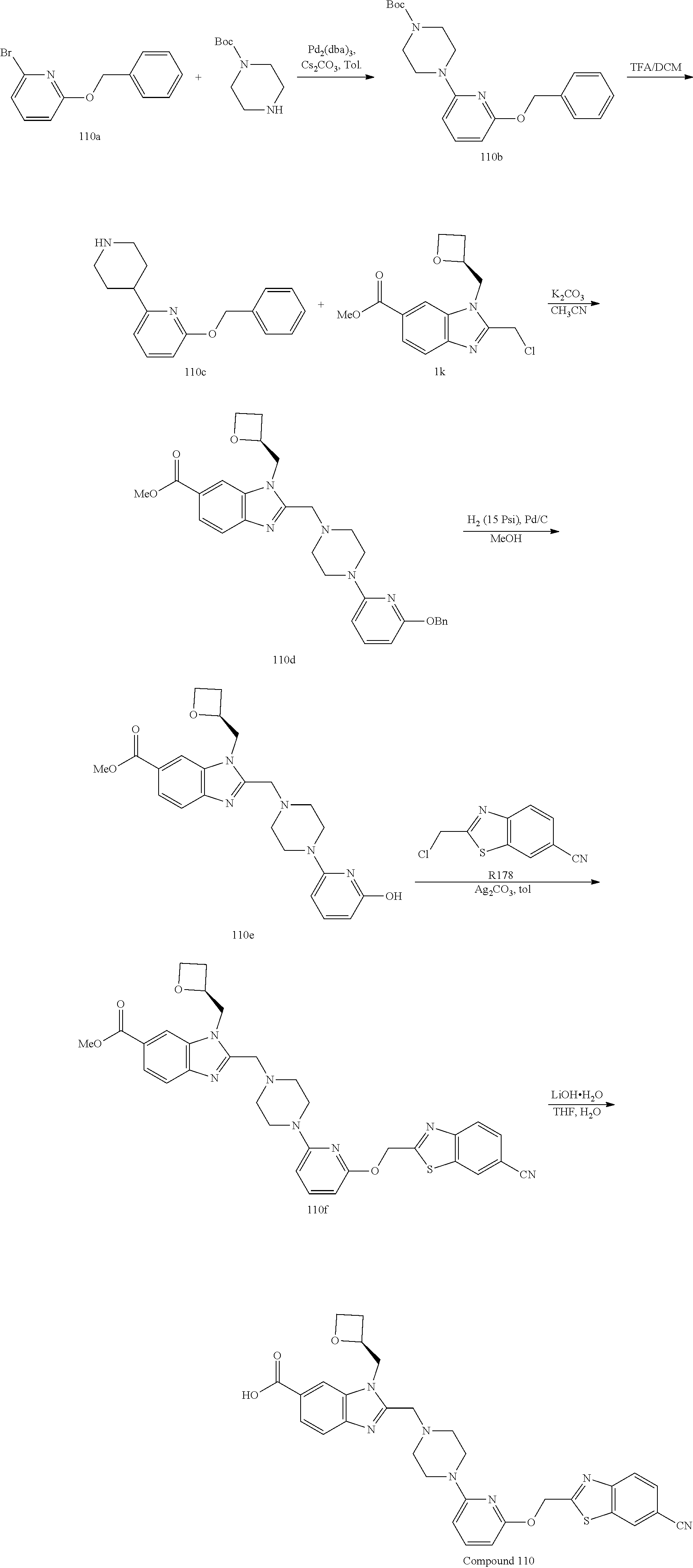

### Tert-butyl 4-(6-(benzyloxy)pyridin-2-yl)piperazine-1-carboxylate (110b)

To a mixture of 2-(benzyloxy)-6-bromopyridine (110a, 0.5 g, 1.89 mmol) and tert-butyl piperazine-1-carboxylate (387.85 mg, 2.08 mmol) in Toluene (10 mL) was added BINAP (117.88 mg, 189.31 umol), Pd2(dba)3 (86.68 mg, 94.65 umol, 0.05 eq) and Cs2CO3 (1.23 g, 3.79 mmol) at 20° C. Then the mixture was degassed and refilled with N2 for 3 times. Then the mixture was stirred at 120° C. for 16 hours. TLC (Petroleum ether:Ethyl acetate=3:1, Rf=0.5) showed 110a was consumed, and one major new spot was formed. The mixture was cooled to 20° C. and washed with H2O (5 mL). The organic layer was dried over Na2SO4, filtered and concentrated in vacuum. The residue was purified by column silicagel chromatography (Petroleum ether:Ethyl acetate=1:0 to 5:1) to give 110b as yellow oil. 1H NMR (400 MHz, CDCl3-d) δ ppm 7.40-7.47 (m, 3H), 7.37 (t, J=7.4 Hz, 2H), 7.31 (d, J=7.0 Hz, 1H), 6.18 (dd, J=8.0, 2.6 Hz, 2H), 3.51 (br d, J=4.2 Hz, 8H), 1.50 (s, 9H).

### 2-(benzyloxy)-6-(piperidin-4-yl)pyridine (110c)

To a mixture of tert-butyl 4-(6-(benzyloxy)pyridin-2-yl)piperazine-1-carboxylate (110b, 0.5 g, 1.35 mmol) in DCM (10 mL) was added TFA (2 mL). Then the mixture was stirred at 15° C. for 16 hours. TLC (Petroleum ether:Ethyl acetate=5:1, Rf=0) showed the reaction was completed. The mixture was concentrated in vacuum, and the residue was extracted with Ethyl acetate (10 mL*2) and saturated NaHCO3 solution (5 mL). The combined organic layer was dried over Na2SO4, filtered and concentrated in vacuum to give 110c as light yellow oil. 1H NMR (400 MHz, CDCl3-d) δ ppm 7.28-7.57 (m, 6H), 6.16-6.36 (m, 2H), 5.31 (s, 2H), 3.65-3.94 (m, 4H), 3.22 (br s, 4H).

### (S)-methyl 2-((4-(6-(benzyloxy)pyridin-2-yl)piperazin-1-yl)methyl)-1-(oxetan-2-ylmethyl)-1H-benzo[d]imidazole-6-carboxylate (110d)

To the solution of (S)-methyl 2-((4-(6-hydroxypyridin-2-yl)piperazin-1-yl)methyl)-1-(oxetan-2-ylmethyl)-1H-benzo[d]imidazole-6-carboxylate (110c, 500 mg, 1.86 mmol) and (S)-methyl 2-(chloromethyl)-1-(oxetan-2-ylmethyl)-1H-benzo[d]imidazole-6-carboxylate (1k, 519.78 mg, 1.76 mmol, 0.95 eq) in CH3CN (6 mL) was added K2CO3 (1.28 g, 9.28 mmol) at 20° C. Then the solution was stirred at 50° C. for 8 hours. LCMS detected desired mass and showed 110c was consumed. The mixture was filtered and the filtrate was concentrated in vacuum. The residue was purified by column chromatography (SiO2, Petroleum ether:Ethyl acetate=80:1 to 20:1) to give 110d as a white solid. 1H NMR (400 MHz, CD3OD-d4) δ ppm 8.34 (s, 1H), 7.96 (dd, J=1.2, 8.4 Hz, 1H), 7.68 (d, J=8.4 Hz, 1H), 7.49-7.18 (m, 6H), 6.24 (d, J=8.0 Hz, 1H), 6.10 (d, J=7.8 Hz, 1H), 5.33-5.20 (m, 3H), 4.85 (br d, J=7.2 Hz, 1H), 4.71 (br dd, J=2.2, 15.2 Hz, 1H), 4.62 (br d, J=6.2 Hz, 1H), 4.45 (td, J=5.8, 9.2 Hz, 1H), 4.10 (q, J=7.0 Hz, 1H), 4.04-3.98 (m, 1H), 3.95-3.84 (m, 4H), 3.49 (br t, J=4.6 Hz, 4H), 2.84-2.72 (m, 1H), 2.67-2.43 (m, 5H).

### (S)-methyl 2-((4-(6-hydroxypyridin-2-yl)piperazin-1-yl)methyl)-1-(oxetan-2-ylmethyl)-1H-benzo[d]imidazole-6-carboxylate (110e)

Pd/C (20 mg, 540.17 umol, 10% purity) was added to the solution of (S)-methyl 2-((4-(6-(benzyloxy)pyridin-2-yl)piperazin-1-yl)methyl)-1-(oxetan-2-ylmethyl)-1H-benzo[d]imidazole-6-carboxylate (110d, 285 mg, 540.17 umol) in MeOH (7 mL) at 20° C. Then the solution was stirred at 20° C. for 16 hours under H2 (15 Psi). LCMS detected the desired mass and showed that 110d was consumed. The mixture was filtered and the filtrate was concentrated in vacuum to give 110e as a yellow solid. 1H NMR (400 MHz, MeOD-d4) δ ppm 8.35 (d, J=0.8 Hz, 1H), 7.97 (dd, J=1.4, 8.5 Hz, 1H), 7.70 (d, J=8.4 Hz, 1H), 7.43 (t, J=8.2 Hz, 1H), 5.93 (d, J=8.4 Hz, 1H), 5.82 (br d, J=7.6 Hz, 1H), 5.26 (br dd, J=2.2, 7.2 Hz, 1H), 4.86 (br d, J=7.2 Hz, 1H), 4.77-4.70 (m, 1H), 4.64 (br d, J=6.0 Hz, 1H), 4.46 (td, J=5.8, 9.1 Hz, 1H), 4.11-4.02 (m, 1H), 3.99-3.90 (m, 4H), 2.87-2.75 (m, 1H), 2.72-2.59 (m, 4H), 2.57-2.46 (m, 1H).

### (S)-methyl 2-((4-(6-((6-cyanobenzo[d]thiazol-2-yl)methoxy)pyridin-2-yl)piperazin-1-yl)methyl)-1-(oxetan-2-ylmethyl)-1H-benzo[d]imidazole-6-carboxylate (110f)

Ag2CO3 (151.27 mg, 548.58 umol, 24.88 uL) was added to the solution of (S)-methyl 2-((4-(6-((6-cyanobenzo[d]thiazol-2-yl)methoxy)pyridin-2-yl)piperazin-1-yl)methyl)-1-(oxetan-2-ylmethyl)-1H-benzo[d]imidazole-6-carboxylate (110e, 120 mg, 274.29 umol) and 2-(chloromethyl)benzo[d]thiazole-6-carbonitrile (62.96 mg, 301.72 umol) in toluene (6 mL) at 20° C. Then the solution was stirred at 120° C. for 8 hours. LCMS detected the desired mass and showed that the reaction was complete. The mixture was filtered and the filtrate was concentrated under reduced pressure. The residue was purified by prep-TLC (Ethyl acetate:Methanol=20:1) to give 110f as a yellow solid. 1H NMR (400 MHz, CD3OD-d4) δ ppm 8.44 (d, J=1.0 Hz, 1H), 7.97 (dd, J=1.4, 8.6 Hz, 1H), 7.76 (dd, J=1.6, 8.6 Hz, 1H), 7.50 (t, J=8.0 Hz, 1H), 5.74 (s, 2H), 5.27-5.18 (m, 1H), 4.87 (br d, J=7.2 Hz, 1H), 4.69 (dd, J=2.4, 15.6 Hz, 1H), 4.61 (s, 1H), 4.44 (td, J=5.8, 9.2 Hz, 1H), 4.10 (q, J=7.0 Hz, 1H), 3.94 (s, 4H), 3.88-3.81 (m, 1H), 3.44 (t, J=5.0 Hz, 4H), 2.82-2.71 (m, 1H), 2.55-2.41 (m, 5H).

### (S)-2-((4-(6-((6-cyanobenzo[d]thiazol-2-yl)methoxy)pyridin-2-yl)piperazin-1-yl)methyl)-1-(oxetan-2-ylmethyl)-1H-benzo[d]imidazole-6-carboxylic acid (Compound 110)

LiOH·H2O (9.63 mg, 229.62 umol) was added to the solution of (S)-methyl 2-((4-(6-((6-cyanobenzo[d]thiazol-2-yl)methoxy)pyridin-2-yl)piperazin-1-yl)methyl)-1-(oxetan-2-ylmethyl)-1H-benzo[d]imidazole-6-carboxylate (110f, 70 mg, 114.81 umol) in THF (7 mL) and H2O (3 mL) at 20° C. Then the solution was stirred at 20° C. for 16 hours. LCMS detected the desired mass and showed 110f was consumed. The mixture was adjusted to pH=7 with HOAc. The mixture was extracted with Ethyl acetate (10 mL*3). The combined organic layers was washed with brine (15 mL), dried over Na2SO4, filtered and concentrated under reduce pressure. The residue was purified by prep-HPLC (Neutral condition, Phenomenex Gemini-NX C18 75*30 mm*3 um; mobile phase: [water (10 mM NH4HCO3)-ACN]; B %: 15%-55%, 8 min) to give Compound 110 as a light yellow solid. 1H NMR (400 MHz, MeOD-d4) δ ppm 8.45 (d, J=1.3 Hz, 1H), 8.33 (d, J=1.0 Hz, 1H), 8.06 (d, J=8.6 Hz, 1H), 7.98 (dd, J=1.4, 8.4 Hz, 1H), 7.77 (dd, J=1.6, 8.4 Hz, 1H), 7.69 (d, J=8.4 Hz, 1H), 7.50 (t, J=7.8 Hz, 1H), 6.32 (d, J=8.0 Hz, 1H), 6.23 (d, J=7.8 Hz, 1H), 5.74 (s, 2H), 5.24 (dq, J=2.4, 7.2 Hz, 1H), 4.83 (br s, 1H), 4.70 (dd, J=2.6, 15.4 Hz, 1H), 4.66-4.59 (m, 1H), 4.45 (td, J=5.8, 9.2 Hz, 1H), 4.01-3.93 (m, 1H), 3.89-3.81 (m, 1H), 3.45 (br t, J=4.8 Hz, 4H), 2.83-2.72 (m, 1H), 2.56-2.44 (m, 5H).

### Example 111 (General Procedure JJ)

### (S)-2-((4-(4-cyano-3-((4-cyano-2-fluorobenzyl)oxy)-1H-pyrazol-1-yl)piperidin-1-yl)methyl)-1-(oxetan-2-ylmethyl)-1H-benzo[d]imidazole-6-carboxylic acid

The title compound was prepared according to Scheme 17. This General Procedure JJ exemplifies Scheme 17 and provides particular synthetic details as applied to the title compound.

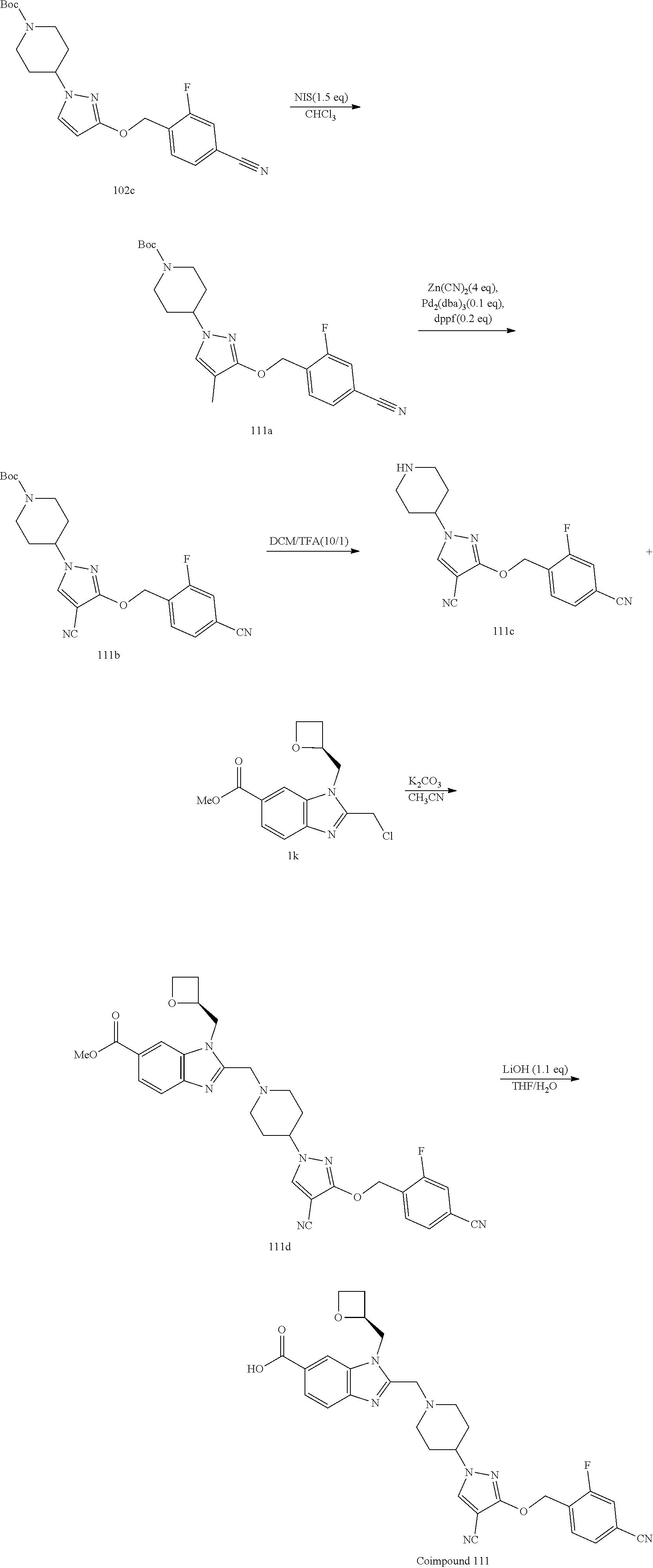

### Tert-butyl 4-(3-((4-cyano-2-fluorobenzyl)oxy)-4-iodo-1H-pyrazol-1-yl)piperidine-1-carboxylate (111a)

To a solution of tert-butyl 4-[3-[(4-cyano-2-fluoro-phenyl)methoxy]pyrazol-1-yl]piperidine-1-carboxylate (108c, 700 mg, 1.75 mmol) in CH3C1 (15 mL) was added NIS (589.93 mg, 2.62 mmol) at 20° C. The mixture was stirred at 60° C. for 2 h. TLC (Petroleum ether:Ethyl acetate=3:1, Rf=0.5) indicated 108c was consumed completely and one new spot was formed. The mixture was concentrated under reduced pressure. The residue was purified by column chromatography (SiO2, Petroleum ether/Ethyl acetate=5/1 to 3/1) to give 111a as a white solid. 1H NMR (400 MHz, CHLOROFORM-d) δ 7.72 (t, J=7.4 Hz, 1H), 7.50 (d, J=7.6 Hz, 1H), 7.38 (dd, J=1.2, 9.4 Hz, 1H), 7.29-7.27 (m, 1H), 5.38 (s, 2H), 4.33-4.17 (m, 2H), 4.17-4.01 (m, 1H), 2.95-2.77 (m, 2H), 2.04 (br d, J=10.0 Hz, 2H), 1.88-1.74 (m, 2H), 1.48 (s, 9H).

### Tert-butyl 4-(4-cyano-3-((4-cyano-2-fluorobenzyl)oxy)-1H-pyrazol-1-yl)piperidine-1-carboxylate (111b)

To a mixture of tert-butyl 4-(4-cyano-3-((4-cyano-2-fluorobenzyl)oxy)-1H-pyrazol-1-yl)piperidine-1-carboxylate (111a, 100 mg, 189.99 umol) in DMF (1.5 mL) was added Zn(CN)2 (89.24 mg, 759.96 umol, 48.24 uL), DPPF (21.07 mg, 38.00 umol) and Pd2(dba)3 (17.40 mg, 19.00 umol) under N2. The mixture was stirred at 100° C. for 2 hours. TLC (Petroleum ether:Ethyl acetate=1:1, Rf=0.40) indicated the starting material was consumed completely and many new spots were formed. The reaction mixture was filtered. The filtrate was poured into water (20 mL) and extracted with ethyl acetate (30 mL*2). The combined organic phase was washed with brine (30 mL*2), dried with anhydrous Na2SO4, filtered and concentrated in vacuum. The residue was purified by prep-TLC (SiO2, Petroleum ether/Ethyl acetate=1/1) to give 111b as a light yellow solid. 1H NMR (400 MHz, MeOD-d4) δ 8.10 (s, 1H), 7.72 (t, J=7.6 Hz, 1H), 7.60 (d, J=8.4 Hz, 2H), 5.43 (s, 2H), 4.27-4.06 (m, 3H), 3.01-2.84 (m, 2H), 2.02 (br d, J=10.4 Hz, 2H), 1.83 (dq, J=4.6, 12.2 Hz, 2H), 1.47 (s, 9H).

### 3-((4-cyano-2-fluorobenzyl)oxy)-1-(piperidin-4-yl)-1H-pyrazole-4-carbonitrile (111c)

To a mixture of tert-butyl 4-(4-cyano-3-((4-cyano-2-fluorobenzyl)oxy)-1H-pyrazol-1-yl)piperidine-1-carboxylate (111b, 50 mg, 117.52 umol) in DCM (3 mL) was added TFA (0.3 mL) under N2. The mixture was stirred at 20° C. for 1 hour. TLC (Petroleum ether:Ethyl acetate=1:1, Rf=0) indicated the starting material was consumed completely and one new spot was formed. The reaction mixture was concentrated under reduced pressure to give 111c as brown oil. The product was used directly in next step.

### (S)-methyl 2-((4-(4-cyano-3-((4-cyano-2-fluorobenzyl)oxy)-1H-pyrazol-1-yl)piperidin-1-yl)methyl)-1-(oxetan-2-ylmethyl)-1H-benzo[d]imidazole-6-carboxylate (111d)

To a mixture of 3-((4-cyano-2-fluorobenzyl)oxy)-1-(piperidin-4-yl)-1H-pyrazole-4-carbonitrile (111c, 45 mg, 138.32 umol) and (S)-methyl 2-(chloromethyl)-1-(oxetan-2-ylmethyl)-1H-benzo[d]imidazole-6-carboxylate (1k, 36.69 mg, 124.49 umol) in CH3CN (2 mL) was added K2CO3 (57.35 mg, 414.95 umol) under N2. The mixture was stirred at 60° C. for 16 hours. TLC (Ethyl acetate:Methanol=10:1, Rf=0.40) indicated the starting material was consumed completely and one new spot was formed. The residue was poured into water (10 mL) and extracted with ethyl acetate (20 mL*3). The combined organic phase was washed with brine (20 mL*2), dried with anhydrous Na2SO4, filtered and concentrated in vacuum. The residue was purified by prep-TLC (SiO2, EA:MeOH=10:1) to give 111d as an off white solid. 1H NMR (400 MHz, CDCl3-d) δ 8.15 (s, 1H), 7.99 (d, J=8.6 Hz, 1H), 7.79-7.65 (m, 2H), 7.62 (s, 1H), 7.50 (d, J=7.6 Hz, 1H), 7.39 (d, J=9.4 Hz, 1H), 5.40 (s, 2H), 5.22 (br d, J=4.4 Hz, 1H), 4.73-4.59 (m, 3H), 4.37 (td, J=6.0, 9.2 Hz, 1H), 4.07-3.99 (m, 2H), 3.96 (s, 4H), 3.04 (br d, J=9.4 Hz, 2H), 2.80-2.69 (m, 1H), 2.50-2.27 (m, 3H), 2.08 (br s, 2H), 2.02-1.90 (m, 2H).

### (S)-2-((4-(4-cyano-3-((4-cyano-2-fluorobenzyl)oxy)-1H-pyrazol-1-yl)piperidin-1-yl)methyl)-1-(oxetan-2-ylmethyl)-1H-benzo[d]imidazole-6-carboxylic acid (Compound 111)

To a mixture of (S)-methyl 2-((4-(4-cyano-3-((4-cyano-2-fluorobenzyl)oxy)-1H-pyrazol-1-yl)piperidin-1-yl)methyl)-1-(oxetan-2-ylmethyl)-1H-benzo[d]imidazole-6-carboxylate (111d, 50 mg, 85.67 umol) in THF (2.8 mL) was added LiOH·H2O (3.95 mg, 94.24 umol) in H2O (1.2 mL) under N2. The mixture was stirred at 20° C. for 16 hours. LCMS showed the starting material was remained and desired mass was detected. The mixture was quenched by addition citric (10%) to just to pH=6-7, and the reaction mixture were concentrated under reduced pressure. The residue was purified by prep-HPLC (column: Waters Xbridge BEH C18 100*30 mm*10 um; mobile phase: [water (10 mM NH4HCO3)-ACN]; B %: 10%-40%, 8 min) to give Compound 111 as a white solid. 1H NMR (400 MHz, MeOD-d4) δ 8.33 (s, 1H), 8.10 (s, 1H), 7.97 (dd, J=1.4, 8.4 Hz, 1H), 7.73 (t, J=7.6 Hz, 1H), 7.67 (d, J=8.6 Hz, 1H), 7.62 (d, J=3.2 Hz, 1H), 7.60 (s, 1H), 5.44 (s, 2H), 5.29-5.23 (m, 1H), 4.90 (br d, J=7.2 Hz, 1H), 4.76-4.69 (m, 1H), 4.69-4.61 (m, 1H), 4.47 (td, J=5.8, 9.2 Hz, 1H), 4.13-4.06 (m, 1H), 4.06-4.00 (m, 1H), 3.97-3.90 (m, 1H), 3.04 (br d, J=11.6 Hz, 1H), 2.94 (br d, J=11.6 Hz, 1H), 2.87-2.77 (m, 1H), 2.62-2.43 (m, 1H), 2.43-2.27 (m, 2H), 2.09-1.93 (m, 4H).

### Example 112

### (S)-2-((4-(6-((5-chlorobenzo[d]thiazol-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((5-chlorobenzo[d]thiazol-2-yl)methoxy)pyridin-2-yl)piperazin-1-yl)methyl)-1-(oxetan-2-ylmethyl)-1H-benzo[d]imidazole-6-carboxylic acid (Compound 112)

1H NMR (400 MHz, MeOD-d4) δ 8.33 (s, 1H), 7.98 (dd, J=1.6, 8.6 Hz, 1H), 7.96-7.92 (m, 2H), 7.66 (s, 1H), 7.49 (t, J=8.0 Hz, 1H), 7.42-7.38 (m, 1H), 6.32 (d, J=8.0 Hz, 1H), 6.21 (d, J=7.8 Hz, 1H), 5.69 (s, 2H), 5.29-5.16 (m, 1H), 4.73-4.67 (m, 1H), 4.67-4.59 (m, 1H), 4.49-4.41 (m, 1H), 4.01-3.82 (m, 2H), 3.45 (br s, 4H), 2.86-2.67 (m, 1H), 2.58-2.44 (m, 5H).

### Example 113

### (S)-2-((4-(6-((6-chlorobenzo[d]thiazol-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((6-chlorobenzo[d]thiazol-2-yl)methoxy)pyridin-2-yl)piperazin-1-yl)methyl)-1-(oxetan-2-ylmethyl)-1H-benzo[d]imidazole-6-carboxylic acid (Compound 113)

1H NMR (400 MHz, CHLOROFORM-d) δ 8.23 (s, 1H), 8.06 (dd, J=1.0, 8.6 Hz, 1H), 7.91 (d, J=8.8 Hz, 1H), 7.84 (d, J=1.8 Hz, 1H), 7.82 (d, J=8.6 Hz, 1H), 7.53-7.38 (m, 2H), 6.25 (br d, J=7.8 Hz, 1H), 6.21 (d, J=7.6 Hz, 1H), 5.72 (s, 2H), 5.28-5.20 (m, 1H), 4.80-4.60 (m, 3H), 4.40 (td, J=6.0 Hz, 1H), 4.01 (s, 2H), 3.50 (br d, J=4.2 Hz, 4H), 2.84-2.69 (m, 1H), 2.63 (br s, 4H), 2.54-2.40 (m, 1H).

### Example 114 (General Procedure KK)

### (S)-2-((4-(3-((4-cyano-2-fluorobenzyl)oxy)-4-methyl-1H-pyrazol-1-yl)piperidin-1-yl)methyl)-1-(oxetan-2-ylmethyl)-1H-benzo[d]imidazole-6-carboxylic acid

The title compound was prepared according to Scheme 17. This General Procedure KK exemplifies Scheme 17 and provides particular synthetic details as applied to the title compound.

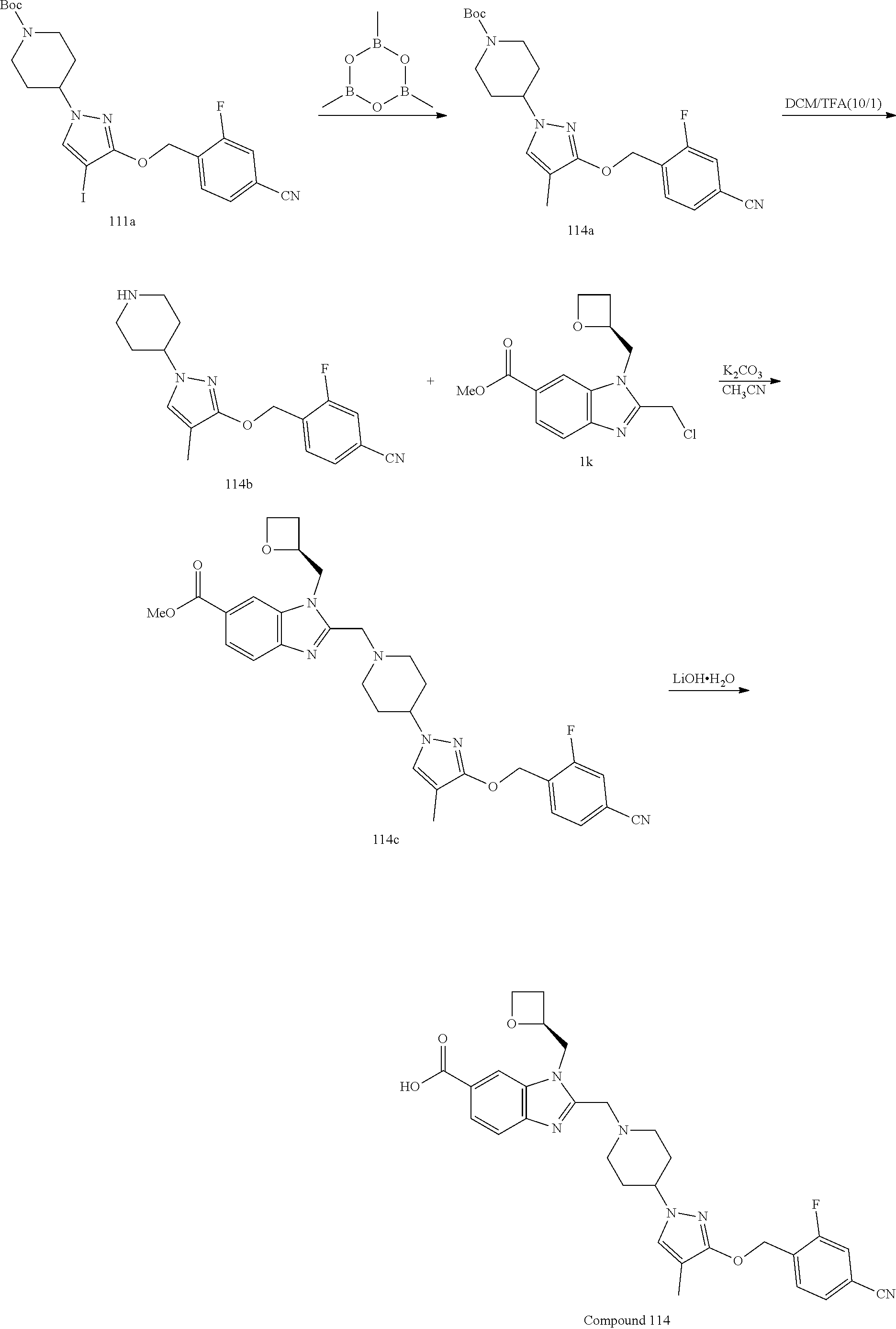

### Tert-butyl 4-(3-((4-cyano-2-fluorobenzyl)oxy)-4-methyl-1H-pyrazol-1-yl)piperidine-1-carboxylate (114a)

To a solution of tert-butyl 4-[3-[(4-cyano-2-fluoro-phenyl)methoxy]-4-iodo-pyrazol-1-yl]piperidine-1-carboxylate (100 mg, 189.99 umol) and 2,4,6-trimethyl-1,3,5,2,4,6-trioxatriborinane (477.01 mg, 1.90 mmol, 531.19 uL, 50% purity, 10 eq) in T-AMYL METHACRYLATE (3 mL) were added [2-(2-aminophenyl)phenyl]-chloro-palladium;dicyclohexyl-[2-(2,6-dimethoxyphenyl)phenyl]phosphane (13.69 mg, 19.00 umol) and Cs2CO3 (123.81 mg, 379.98 umol) at 20° C. under N2. The mixture was stirred at 80° C. for 16 hours. LCMS showed 111a was consumed completely and desired mass was detected. The reaction mixture was diluted with Ethyl acetate (30 mL) and washed with H2O (10 mL), dried over Na2SO4, filtered and concentrated under reduced pressure. The residue was purified by prep-TLC (SiO2, Petroleum ether:Ethyl acetate=2:1) to give 114a as a light yellow solid. 1H NMR (400 MHz, CDCl3-d)) δ 7.69 (t, J=7.4 Hz, 1H), 7.47 (d, J=7.8 Hz, 1H), 7.37 (dd, J=1.2, 9.2 Hz, 1H), 7.03 (s, 1H), 5.34 (s, 2H), 4.18 (br d, J=15.4 Hz, 2H), 3.99 (tt, J=3.8, 11.2 Hz, 1H), 2.86 (br t, J=12.6 Hz, 2H), 2.08-2.00 (m, 2H), 1.87-1.72 (m, 2H), 1.48 (s, 9H).

### 3-fluoro-4-(((4-methyl-1-(piperidin-4-yl)-1H-pyrazol-3-yl)oxy)methyl)benzonitrile (114b)

A solution of tert-butyl 4-[3-[(4-cyano-2-fluoro-phenyl)methoxy]-4-methyl-pyrazol-1-yl]piperidine-1-carboxylate (114a, 50 mg, 120.64 umol) in TFA (0.2 mL) and DCM (2 mL) was stirred at 20° C. for 1 hour. LCMS showed 114a was consumed completely and desired mass was detected. The mixture was concentrated under reduced pressure to give 114b. The product was used directly in next step. ¹H NMR (400 MHz, CHLOROFORM-d) δ 7.67 (t, J=7.4 Hz, 1H), 7.48 (br d, J=7.6 Hz, 1H), 7.38 (br d, J=9.2 Hz, 1H), 7.06 (s, 1H), 5.33 (s, 2H), 4.18 (br s, 1H), 3.58 (br s, 2H), 3.14 (br s, 2H), 2.28 (br d, J=4.2 Hz, 4H).

### (S)-methyl 2-((4-(3-((4-cyano-2-fluorobenzyl)oxy)-4-methyl-1H-pyrazol-1-yl)piperidin-1-yl)methyl)-1-(oxetan-2-ylmethyl)-1H-benzo[d]imidazole-6-carboxylate (114c)

To a solution of 3-fluoro-4-[[4-methyl-1-(4-piperidyl)pyrazol-3-yl]oxymethyl]benzonitrile (114b, 38 mg, 120.88 umol) and methyl 2-(chloromethyl)-3-[[(2S)-oxetan-2-yl]methyl]benzimidazole-5-carboxylate (1k, 35.63 mg, 120.88 umol) in CH3CN (3 mL) was added K2CO3 (50.12 mg, 362.65 umol) at 20° C. The mixture was stirred at 60° C. for 16 hours. LCMS showed 114b was consumed completely and desired mass was detected. The reaction mixture was diluted with Ethyl acetate (30 mL) and washed with H2O (10 mL), dried over Na2SO4, filtered and concentrated under reduced pressure. The residue was purified by prep-TLC (SiO2, Petroleum ether:Ethyl acetate=1:1) to give 114c as a light yellow solid. 1H NMR (400 MHz, CDCl3-d) δ 8.16 (s, 1H), 7.98 (dd, J=1.4, 8.6 Hz, 1H), 7.76 (d, J=8.6 Hz, 1H), 7.69 (t, J=7.0 Hz, 1H), 7.47 (d, J=8.2 Hz, 1H), 7.39-7.32 (m, 1H), 7.03 (s, 1H), 5.34 (s, 2H), 5.26-5.19 (m, 1H), 4.76-4.61 (m, 3H), 4.39 (td, J=5.9, 9.2 Hz, 1H), 3.99 (d, J=3.4 Hz, 2H), 3.96 (s, 3H), 3.93-3.84 (m, 1H), 2.99 (br t, J=10.2 Hz, 2H), 2.76 (ddd, J=3.0, 5.6, 10.6 Hz, 1H), 2.51-2.40 (m, 1H), 2.38-2.25 (m, 2H), 2.11-2.01 (m, 2H), 2.01-1.88 (m, 2H).

### (S)-2-((4-(3-((4-cyano-2-fluorobenzyl)oxy)-4-methyl-1H-pyrazol-1-yl)piperidin-1yl)methyl)-1-(oxetan-2-ylmethyl)-1H-benzo[d]imidazole-6-carboxylic acid (114)

To a solution of methyl 2-[[4-[3-[(4-cyano-2-fluoro-phenyl)methoxy]-4-methyl-pyrazol-1-yl]-1-piperidyl]methyl]-3-[[(2S)-oxetan-2-yl]methyl]benzimidazole-5-carboxylate (114c, 50 mg, 87.32 umol) in THF (3.5 mL) and H2O (1.5 mL) was added LiOH·H2O (7.33 mg, 174.63 umol) at 20° C. The mixture was stirred at 20° C. for 16 hours. LC-MS showed 114c was consumed completely and desired mass was detected. The mixture was adjusted to pH=6 with AcOH. Then the mixture was concentrated under reduced pressure to remove THF. The aqueous layer was extracted with i-PrOH/DCM (1/10, 20 mL). The organic layer was concentrated under reduced pressure. The residue was purified by prep-HPLC (column: Phenomenex Gemini-NX C18 75*30 mm*3 um; mobile phase: [water (1OMm NH4HCO3)-ACN]; B %: 25%-45%, 8 min) to give Compound 114 as a white solid. ¹H NMR (400 MHz, CHLOROFORM-d) δ 8.19 (s, 1H), 8.05 (dd, J=1.4, 8.6 Hz, 1H), 7.81 (d, J=8.4 Hz, 1H), 7.69 (t, J=7.4 Hz, 1H), 7.47 (d, J=7.6 Hz, 1H), 7.38-7.28 (m, 1H), 7.04 (s, 1H), 5.34 (s, 2H), 5.26-5.15 (m, 1H), 4.77-4.59 (m, 3H), 4.40 (td, J=5.8, 9.0 Hz, 1H), 4.02 (s, 2H), 3.97-3.80 (m, 1H), 3.02 (br t, J=12.0 Hz, 2H), 2.87-2.62 (m, 1H), 2.51-2.26 (m, 3H), 2.10-1.84 (m, 4H).

### Example 115

### 2-((4-(6-((S)-1-(benzo[d]thiazol-2-yl)ethoxy)pyridin-2-yl)piperazin-1-yl)methyl)-1-(((S)-oxetan-2-yl)methyl)-1H-benzo[d]imidazole-6-carboxylic acid

The title compound was prepared and can be prepared similarly following the procedures described by General Procedure C.

### 2-((4-(6-((S)-1-(benzo[d]thiazol-2-yl)ethoxy)pyridin-2-yl)piperazin-1-yl)methyl)-1-(((S)-oxetan-2-yl)methyl)-1H-benzo[d]imidazole-6-carboxylic acid (Compound 115)

1H NMR (400 MHz, CDCl3) δ 8.23 (s, 1H), 8.06 (d, J=8.8 Hz, 1H), 7.98 (d, J=8.0 Hz, 1H), 7.83 (t, J=8.6 Hz, 2H), 7.43 (t, J=7.8 Hz, 2H), 7.37-7.28 (m, 1H), 6.48 (q, J=6.6 Hz, 1H), 6.23 (d, J=7.8 Hz, 1H), 6.15 (d, J=8.0 Hz, 1H), 5.25-5.18 (m, 1H), 4.76-4.59 (m, 3H), 4.39 (td, J=6.0, 9.0 Hz, 1H), 3.96 (s, 2H), 3.51-3.41 (m, 2H), 3.41-3.21 (m, 2H), 2.77-2.66 (m, 1H), 2.61-2.38 (m, 5H), 1.83 (d, J=6.6 Hz, 3H).

### Example 116

### 2-((4-(6-((R)-1-(benzo[d]thiazol-2-yl)ethoxy)pyridin-2-yl)piperazin-1-yl)methyl)-1-(((S)-oxetan-2-yl)methyl)-1H-benzo[d]imidazole-6-carboxylic acid

The title compound was prepared and can be prepared similarly following the procedures described by General Procedure C.

### 2-((4-(6-((R)-1-(benzo[d]thiazol-2-yl)ethoxy)pyridin-2-yl)piperazin-1-yl)methyl)-1-(((S)-oxetan-2-yl)methyl)-1H-benzo[d]imidazole-6-carboxylic acid (Compound 116)

1H NMR (400 MHz, CDCl3) δ 8.23 (s, 1H), 8.06 (br d, J=8.6 Hz, 1H), 7.98 (d, J=8.0 Hz, 1H), 7.88-7.76 (m, 2H), 7.43 (br t, J=8.0 Hz, 2H), 7.39-7.31 (m, 1H), 6.47 (q, J=6.7 Hz, 1H), 6.22 (d, J=7.8 Hz, 1H), 6.15 (d, J=8.0 Hz, 1H), 5.20 (br d, J=2.4 Hz, 1H), 4.77-4.68 (m, 1H), 4.68-4.54 (m, 2H), 4.44-4.30 (m, 1H), 3.94 (br s, 2H), 3.42 (br s, 4H), 2.81-2.65 (m, 1H), 2.53 (br s, 2H), 2.49-2.35 (m, 6H), 1.83 (d, J=6.6 Hz, 3H).

### Example 117 (General Procedure LL)

### (S)-2-((4-(3-((4-cyano-2-fluorobenzyl)oxy)-4-(trifluoromethyl)-1H-pyrazol-1-yl)piperidin-1-yl)methyl)-1-(oxetan-2-ylmethyl)-1H-benzo[d]imidazole-6-carboxylic acid

The title compound was prepared according to Scheme 17. This General Procedure LL exemplifies Scheme 17 and provides particular synthetic details as applied to the title compound.

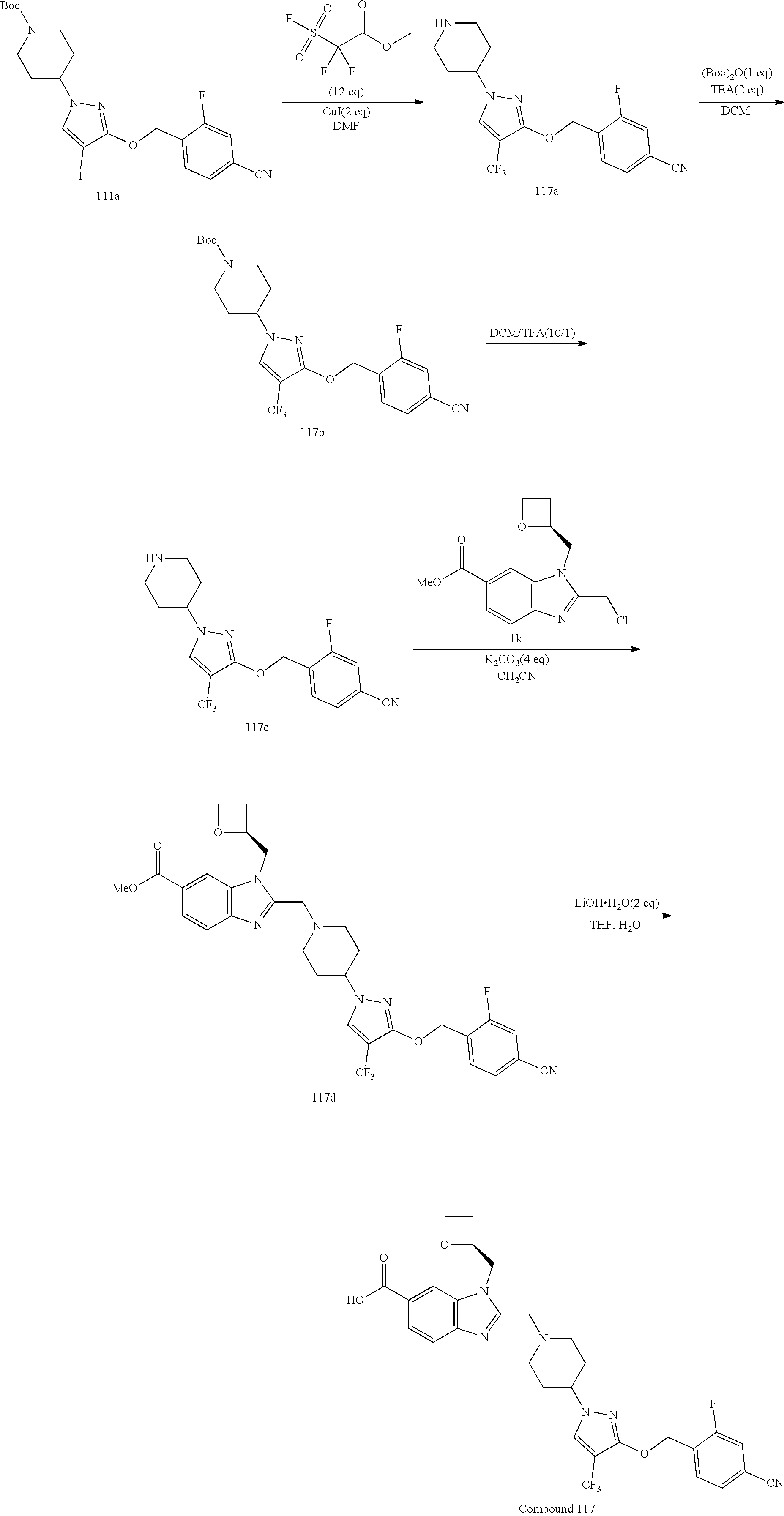

### 3-fluoro-4-(((1-(piperidin-4-yl)-4-(trifluoromethyl)-1H-pyrazol-3-yl)oxy)methyl)benzonitrile (117a)

To a solution of tert-butyl 4-(3-((4-cyano-2-fluorobenzyl)oxy)-4-iodo-1H-pyrazol-1-yl)piperidine-1-carboxylate (111a, 100 mg, 189.99 umol) in DMF (5 mL) was added methyl 2,2-difluoro-2-fluorosulfonyl-acetate (438.00 mg, 2.28 mmol, 290.06 uL, 12 eq) and CuI (72.37 mg, 379.98 umol) at 20° C. under N2. Then the mixture was stirred at 100° C. for 16 hours. LCMS showed 111a was consumed completely and desired mass was detected. The mixture was concentrated under reduced pressure to give 117a as a brown solid.

### Tert-butyl 4-(3-((4-cyano-2-fluorobenzyl)oxy)-4-(trifluoromethyl)-1H-pyrazol-1-yl)piperidine-1-carboxylate (117b)

To a solution of 3-fluoro-4-(((1-(piperidin-4-yl)-4-(trifluoromethyl)-1H-pyrazol-3-yl)oxy)methyl)benzonitrile (117a, 65 mg, 176.47 umol) in DCM (3 mL) was added Boc2O (38.52 mg, 176.47 umol, 40.54 uL) and TEA (35.71 mg, 352.95 umol, 49.13 uL) at 20° C. The mixture was stirred at 20° C. for 1 hour. LCMS showed 117a was consumed completely and desired mass was detected. The suspension was filtered through a pad of Celite and the pad cake was washed with Ethyl acetate (20 mL*3). The combined filtrates were concentrated under reduced pressure to give 117b as white oil. 1H NMR (400 MHz, CHLOROFORM-d) δ 7.68 (br t, J=7.6 Hz, 1H), 7.56-7.46 (m, 2H), 7.38 (br d, J=9.2 Hz, 1H), 5.41 (s, 2H), 4.24 (br dd, J=1.8, 5.0 Hz, 2H), 4.05 (br t, J=11.0 Hz, 1H), 2.95-2.80 (m, 2H), 2.17-2.00 (m, 2H), 1.93-1.76 (m, 2H), 1.49 (d, J=1.4 Hz, 9H).

### 3-fluoro-4-(((1-(piperidin-4-yl)-4-(trifluoromethyl)-1H-pyrazol-3-yl)oxy)methyl)benzonitrile (117c)

To a solution of tert-butyl 4-(3-((4-cyano-2-fluorobenzyl)oxy)-4-(trifluoromethyl)-1H-pyrazol-1-yl)piperidine-1-carboxylate (117b, 45 mg, 96.06 umol) in DCM (2 mL) was added TFA (0.2 mL) at 20° C. The mixture was stirred at 20° C. for 1 hour. LCMS showed 117b was consumed completely and desired mass was detected. The mixture was concentrated under reduced pressure to remove DCM. The residue was diluted with NaHCO3 (aq) 20 mL and extracted with Ethyl acetate (20 mL *2). The combined organic layers were washed with brine (15 mL*3), dried with anhydrous Na2SO4, filtered and concentrated under reduced pressure to give 117c as a yellow solid. 1H NMR (400 MHz, METHANOL-d4) δ 7.89 (s, 1H), 7.77-7.65 (m, 1H), 7.59 (br d, J=8.6 Hz, 2H), 5.42 (s, 2H), 4.24-4.06 (m, 1H), 3.15-3.15 (m, 1H), 3.18 (br d, J=12.8 Hz, 1H), 2.75 (br t, J=12.2 Hz, 2H), 2.12-1.99 (m, 2H), 1.98-1.83 (m, 2H).

### (S)-methyl 2-((4-(3-((4-cyano-2-fluorobenzyl)oxy)-4-(trifluoromethyl)-1H-pyrazol-1-yl)piperidin-1-yl)methyl)-1-(oxetan-2-ylmethyl)-1H-benzo[d]imidazole-6-carboxylate (117d)

To a mixture of 3-fluoro-4-(((1-(piperidin-4-yl)-4-(trifluoromethyl)-1H-pyrazol-3-yl)oxy)methyl)benzonitrile (117c, 35 mg, 95.02 umol) in CH3CN (3 mL) was added K2CO3 (52.53 mg, 380.10 umol) at 20° C. under N2 for 0.5 hour. Then (S)-methyl 2-(chloromethyl)-1-(oxetan-2-ylmethyl)-1H-benzo[d]imidazole-6-carboxylate (1k, 30.81 mg, 104.53 umol) was added to the mixture one portion. The mixture was stirred at 50° C. for 15.5 hours. TLC (Ethyl acetate:Methanol=20:1, Rf=0.4) showed 117c was comsumed completely. The mixture was diluted with H2O (15 mL) and extracted with Ethyl acetate (20 mL*2). The combined organic layers were washed with brine (15 mL*2), dried with anhydrous Na2SO4, filtered and concentrated under reduced pressure. The residue was purified by prep-TLC (Ethyl acetate:Methanol=20:1, Rf=0.4) to give 117d as a yellow solid. 1H NMR (400 MHz, CDCl3-d) δ 8.15 (s, 1H), 7.99 (dd, J=1.4, 8.4 Hz, 1H), 7.77 (d, J=8.4 Hz, 1H), 7.67 (t, J=7.4 Hz, 1H), 7.52-7.45 (m, 2H), 7.37 (dd, J=1.2, 9.4 Hz, 1H), 5.41 (s, 2H), 5.28-5.18 (m, 1H), 4.73-4.60 (m, 3H), 4.38 (td, J=5.8, 9.2 Hz, 1H), 4.02 (d, J=7.8 Hz, 2H), 3.96 (s, 4H), 3.10-2.97 (m, 2H), 2.82-2.70 (m, 1H), 2.54-2.27 (m, 3H), 2.15-2.07 (m, 3H), 2.03-1.89 (m, 2H).

### (S)-2-((4-(3-((4-cyano-2-fluorobenzyl)oxy)-4-(trifluoromethyl)-1H-pyrazol-1-yl)piperidin-1-yl)methyl)-1-(oxetan-2-ylmethyl)-1H-benzo[d]imidazole-6-carboxylic acid (Compound 117)

To a solution of (S)-methyl 2-((4-(3-((4-cyano-2-fluorobenzyl)oxy)-4-(trifluoromethyl)-1H-pyrazol-1-yl)piperidin-1-yl)methyl)-1-(oxetan-2-ylmethyl)-1H-benzo[d]imidazole-6-carboxylate (117d, 40 mg, 63.84 umol) in THF (2.1 mL) and H2O (0.9 mL) was added LiOH·H2O (5.36 mg, 127.67 umol, 75.67 uL) at 25° C. The mixture was stirred at 25° C. for 16 hours. LCMS showed 117d was remained and desired mass was detected. The mixture was adjusted to pH=6 with Citric acid (1 M). Then the mixture was diluted with H2O (15 mL) and extracted with Ethyl acetate (20 mL*2). The combined organic layers were washed with brine (10 mL*3), dried with anhydrous Na2SO4, filtered and concentrated under reduced pressure. The residue was purified by Prep-HPLC (column: 3_Phenomenex Luna C18 75*30 mm*3 um; mobile phase: [water (10 mM NH4HCO3)-ACN]; B %: 20%-50%, 9 min) to give Compound 117 as a white solid. 1H NMR (400 MHz, CDCl3-d) δ 8.21 (s, 1H), 8.05 (br d, J=8.4 Hz, 1H), 7.81 (d, J=8.4 Hz, 1H), 7.67 (t, J=7.4 Hz, 1H), 7.55-7.45 (m, 2H), 7.37 (d, J=9.2 Hz, 1H), 5.41 (s, 2H), 5.24 (br s, 1H), 4.79-4.60 (m, 3H), 4.39 (td, J=6.0, 9.1 Hz, 1H), 4.11-3.90 (m, 3H), 3.03 (br d, J=8.0 Hz, 2H), 2.84-2.71 (m, 2H), 2.55-2.27 (m, 3H), 2.17-1.90 (m, 5H).

### Example 118 (General Procedure MM)

### (S)-2-((4-(3-((4-cyano-2-fluorobenzyl)oxy)-4-isopropyl-1H-pyrazol-1-yl)piperidin-1-yl)methyl)-1-(oxetan-2-ylmethyl)-1H-benzo[d]imidazole-6-carboxylic acid

The title compound was prepared according to Scheme 17. This General Procedure MM exemplifies Scheme 17 and provides particular synthetic details as applied to the title compound.

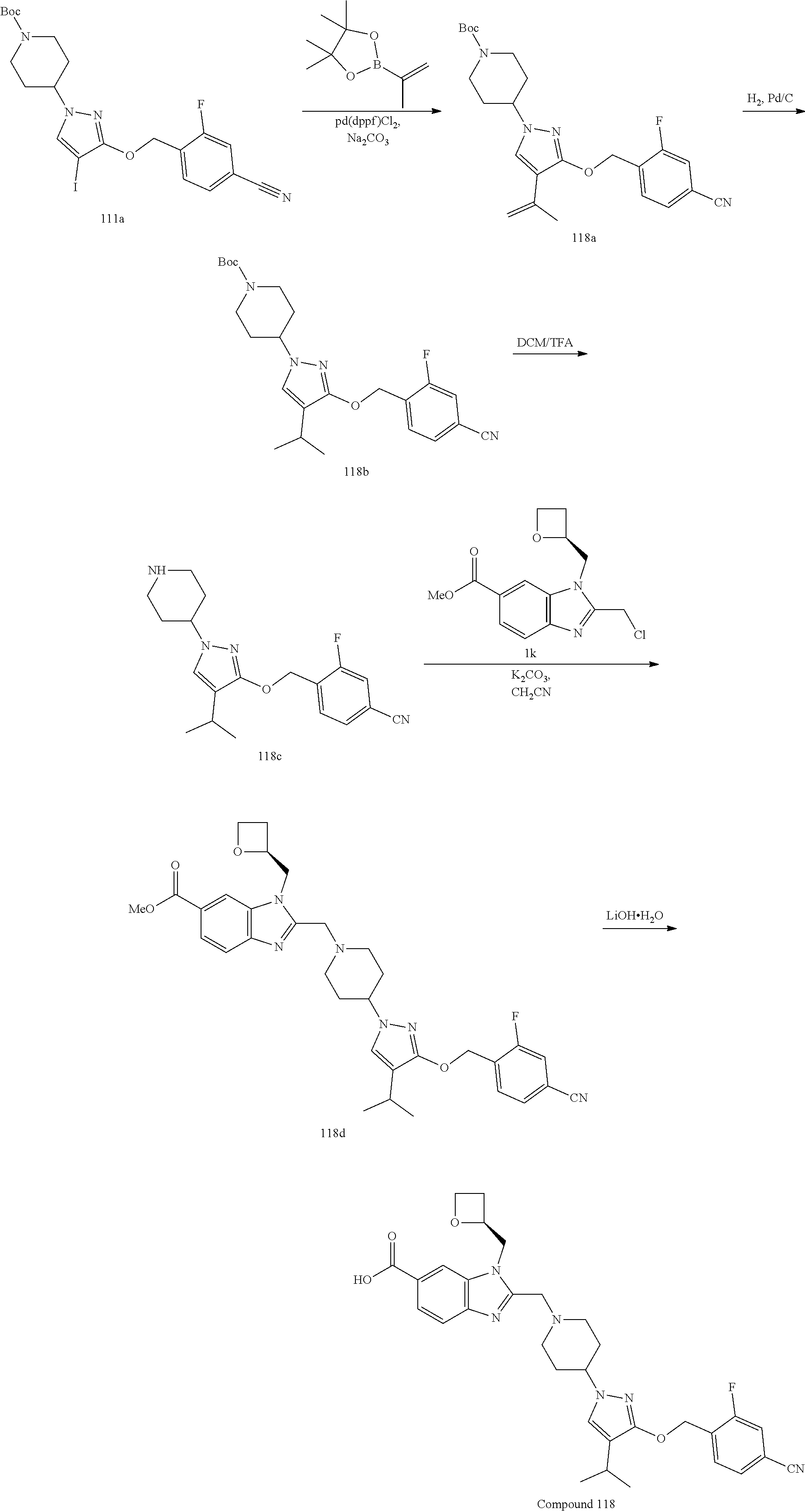

Tert-butyl 4-(3-((4-cyano-2-fluorobenzyl)oxy)-4-(prop-1-en-2-yl)-1H-pyrazol-1-yl)piperidine-1-carboxylate (118a).

To a mixture of tert-butyl 4-(3-((4-cyano-2-fluorobenzyl)oxy)-4-(prop-1-en-2-yl)-1H-pyrazol-1-yl)piperidine-1-carboxylate (111a, 100 mg, 189.99 umol) and 2-isopropenyl-4,4,5,5-tetramethyl-1,3,2-dioxaborolane (159.63 mg, 949.95 umol) in dioxane (3 mL) was added cyclopentyl(diphenyl)phosphane;dichloromethane;dichloropalladium;iron (15.52 mg, 19.00 umol) and Na2CO3 (2 M, 949.95 uL, 10 eq) at 20° C. under N2. The mixture was stirred at 80° C. for 16 hours. LCMS showed 111a was consumed completely and desired mass was detected. The suspension was filtered through a pad of Celite and the pad cake was washed with Ethyl acetate (20 mL*3). The combined filtrates were concentrated under reduced pressure. The residue was purified by prep-TLC (Petroleum ether:Ethyl acetate=3:1) to give 118a as a yellow solid. 1H NMR (400 MHz, CHLOROFORM-d) δ 7.69 (t, J=7.4 Hz, 1H), 7.48 (dd, J=1.2, 7.8 Hz, 1H), 7.38 (dd, J=1.2, 9.2 Hz, 1H), 7.22 (s, 1H), 5.48 (d, J=1.2 Hz, 1H), 5.43 (s, 2H), 4.97-4.90 (m, 1H), 4.31-4.15 (m, 2H), 4.08-3.96 (m, 1H), 2.87 (br t, J=12.0 Hz, 2H), 2.11-1.99 (m, 5H), 1.93-1.76 (m, 2H), 1.49 (s, 9H).

### Tert-butyl 4-(3-((4-cyano-2-fluorobenzyl)oxy)-4-isopropyl-1H-pyrazol-1-yl)piperidine-1-carboxylate (118b)

To a solution of tert-butyl 4-(3-((4-cyano-2-fluorobenzyl)oxy)-4-(prop-1-en-2-yl)-1H-pyrazol-1-yl)piperidine-1-carboxylate (118a, 50 mg, 113.50 umol) in Ethyl acetate (1 mL) was added Pd/C (50 mg, 100.00 umol, 10% purity, 8.81e-1 eq) under N2 atmosphere. The suspension was degassed and purged with H2 for 3 times. The mixture was stirred under H2 (15 Psi) at 20° C. for 5 min. LCMS showed 118a was consumed completely and desired mass was detected. The suspension was filtered through a pad of Celite and the pad cake was washed with Ethyl acetate (20 mL*3). And the mixture was filtered and concentrated under reduced pressure to give 118b as a white solid. 1H NMR (400 MHz, CHLOROFORM-d) δ 7.68 (t, J=7.5 Hz, 1H), 7.48 (d, J=7.8 Hz, 1H), 7.37 (dd, J=0.8, 9.2 Hz, 1H), 7.00 (s, 1H), 5.35 (s, 2H), 4.20 (br d, J=3.0 Hz, 2H), 4.06-3.90 (m, 1H), 2.95-2.73 (m, 2H), 2.13-1.99 (m, 2H), 1.90-1.73 (m, 2H), 1.48 (s, 9H), 1.20 (d, J=6.8 Hz, 6H). 3-fluoro-4-(((4-isopropyl-1-(piperidin-4-yl)-1H-pyrazol-3-yl)oxy)methyl)benzonitrile (118c).

To a solution of tert-butyl 4-(3-((4-cyano-2-fluorobenzyl)oxy)-4-isopropyl-1H-pyrazol-1-yl)piperidine-1-carboxylate (118b, 45 mg, 101.69 umol) in DCM (2 mL) was added TFA (0.2 mL) at 20° C. The mixture was stirred at 20° C. for 1 hour. LCMS showed 118b was consumed completely and desired mass was detected. The mixture was concentrated under reduced pressure to remove DCM. The residue was diluted with NaHCO3 (aq, 20 mL) and extracted with Ethyl acetate (20 mL *2). The combined organic layers were washed with brine (15 mL*3), dried with anhydrous Na2SO4, filtered and concentrated under reduced pressure to give 118c as a white solid. 1H NMR (400 MHz, MeOD-d4) δ 7.76-7.66 (m, 1H), 7.64-7.52 (m, 2H), 7.23 (s, 1H), 5.33 (s, 2H), 4.14-3.98 (m, 1H), 3.22 (br d, J=13.1 Hz, 2H), 2.85-2.69 (m, 2H), 2.12-1.99 (m, 2H), 1.98-1.85 (m, 2H), 1.17 (d, J=7.0 Hz, 6H).

### (S)-methyl 2-((4-(3-((4-cyano-2-fluorobenzyl)oxy)-4-isopropyl-1H-pyrazol-1-yl)piperidin-1-yl)methyl)-1-(oxetan-2-ylmethyl)-1H-benzo[d]imidazole-6-carboxylate (118d)

To a mixture of 3-fluoro-4-(((4-isopropyl-1-(piperidin-4-yl)-1H-pyrazol-3-yl)oxy)methyl)benzonitrile (118c, 50 mg, 146.02 umol) in CH3CN (3 mL) was added K2CO3 (80.73 mg, 584.10 umol) at 20° C. under N2 for 0.5 hour. Then (S)-methyl 2-(chloromethyl)-1-(oxetan-2-ylmethyl)-1H-benzo[d]imidazole-6-carboxylate (1k, 47.34 mg, 160.63 umol) was added to the mixture. The mixture was stirred at 50° C. for 15.5 hours. TLC (Ethyl acetate:Methanol=20:1, Rf=0.4) showed 118d was comsumed completely. The mixture was diluted with H2O (15 mL) and extracted with Ethyl acetate (20 mL*2). The combined organic layers were washed with brine (15 mL*2), dried with anhydrous Na2SO4, filtered and concentrated under reduced pressure. The residue was purified by prep-TLC (Ethyl acetate:Methanol=20:1, Rf=0.4) to give 118d as a white solid. 1H NMR (400 MHz, CDCl3-d) δ 8.16 (s, 1H), 7.98 (br d, J=8.6 Hz, 1H), 7.76 (d, J=8.2 Hz, 1H), 7.68 (t, J=7.4 Hz, 1H), 7.47 (br d, J=7.6 Hz, 1H), 7.36 (br d, J=9.2 Hz, 1H), 7.00 (s, 1H), 5.35 (s, 2H), 5.23 (br d, J=1.4 Hz, 1H), 4.79-4.59 (m, 3H), 4.44-4.34 (m, 1H), 4.02-3.94 (m, 5H), 3.93-3.82 (m, 1H), 2.98 (br t, J=9.6 Hz, 2H), 2.85-2.71 (m, 2H), 2.53-2.41 (m, 1H), 2.31 (br d, J=11.0 Hz, 2H), 2.06 (s, 2H), 2.01-1.87 (m, 2H), 1.19 (d, J=6.8 Hz, 6H).

### (S)-2-((4-(3-((4-cyano-2-fluorobenzyl)oxy)-4-isopropyl-1H-pyrazol-1-yl)piperidin-1-yl)methyl)-1-(oxetan-2-ylmethyl)-1H-benzo[d]imidazole-6-carboxylic acid (Compound 118)

To a solution of (S)-methyl 2-((4-(3-((4-cyano-2-fluorobenzyl)oxy)-4-isopropyl-1H-pyrazol-1-yl)piperidin-1-yl)methyl)-1-(oxetan-2-ylmethyl)-1H-benzo[d]imidazole-6-carboxylate (118d, 45 mg, 74.91 umol) in THF (1.4 mL) and H2O (0.6 mL) was added LiOH·H2O (6.29 mg, 149.83 umol, 75.67 uL) at 25° C. The mixture was stirred at 25° C. for 16 hours. LCMS showed 118d was consumed completely and desired mass was detected. The mixture was adjusted to pH=6 with Citric acid (1 M). Then the mixture was diluted with H2O 15 mL and extracted with Ethyl acetate (20 mL *2). The combined organic layers were washed with brine (10 mL*3), dried with anhydrous Na2SO4, filtered and concentrated under reduced pressure. The residue was purified by Prep-HPLC (column: Phenomenex Gemini-NX C18 75*30 mm*3 um; mobile phase: [water (10 mM NH4HCO3)-ACN]; B %: 30%-50%, 6 min) to give Compound 118 as a white solid. 1H NMR (400 MHz, CDCl3-d) δ 8.20 (s, 1H), 8.05 (dd, J=1.2, 8.5 Hz, 1H), 7.81 (d, J=8.4 Hz, 1H), 7.68 (t, J=7.4 Hz, 1H), 7.47 (d, J=7.8 Hz, 1H), 7.36 (d, J=9.2 Hz, 1H), 7.01 (s, 1H), 5.35 (s, 2H), 5.29-5.18 (m, 1H), 4.79-4.60 (m, 3H), 4.46-4.36 (m, 1H), 4.02 (s, 2H), 3.95-3.83 (m, 1H), 3.02 (br t, J=11.6 Hz, 2H), 2.78 (td, J=6.4, 13.6 Hz, 2H), 2.54-2.28 (m, 3H), 2.15-1.88 (m, 4H), 1.19 (d, J=6.8 Hz, 6H).

### Example 119

### (S)-2-((4-(6-((5-fluorobenzo[d]thiazol-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((5-fluorobenzo[d]thiazol-2-yl)methoxy)pyridin-2-yl)piperazin-1-yl)methyl)-1-(oxetan-2-ylmethyl)-1H-benzo[d]imidazole-6-carboxylic acid (Compound 119)

1H NMR (400 MHz, METHANOL-d4) δ 8.33 (s, 1H), 8.01-7.91 (m, 2H), 7.71-7.61 (m, 2H), 7.49 (t, J=8.0 Hz, 1H), 7.21 (dt, J=2.4, 8.8 Hz, 1H), 6.31 (d, J=8.2 Hz, 1H), 6.20 (d, J=7.8 Hz, 1H), 5.69 (s, 2H), 5.28-5.19 (m, 1H), 4.89 (br s, 1H), 4.74-4.67 (m, 1H), 4.66-4.58 (m, 1H), 4.45 (td, J=5.8, 9.0 Hz, 1H), 4.01-3.94 (m, 1H), 3.89-3.82 (m, 1H), 3.46 (br s, 4H), 2.83-2.71 (m, 1H), 2.58-2.44 (m, 5H).

### Example 120

### (S)-2-((4-(6-(5-cyano-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure B.

### (S)-2-((4-(6-(5-cyano-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid (Compound 120)

1H NMR (400 MHz, CHLOROFORM-d) δ 8.25 (s, 1H), 8.05 (d, J=8.6 Hz, 1H), 7.82 (d, J=8.6 Hz, 1H), 7.51 (d, J=7.2 Hz, 1H), 7.42-7.35 (m, 2H), 7.29 (br s, 1H), 6.10 (d, J=7.6 Hz, 1H), 6.03 (d, J=8.4 Hz, 1H), 5.29-5.22 (m, 1H), 4.81-4.62 (m, 4H), 4.83-4.61 (m, 1H), 4.43 (td, J=6.0, 9.2 Hz, 1H), 4.04 (s, 2H), 3.88 (t, J=5.8 Hz, 2H), 3.54 (br s, 4H), 3.13 (t, J=5.6 Hz, 2H), 2.81-2.73 (m, 1H), 2.69 (br t, J=4.6 Hz, 4H), 2.56-2.41 (m, 1H).

### Example 121

### 2-(((1R,6S)-6-(6-((4-cyano-2-fluorobenzyl)oxy)pyridin-2-yl)-3-azabicyclo[4.1.0]heptan-3-yl)methyl)-1-((1-ethyl-1H-imidazol-5-yl)methyl)-1H-benzo[d]imidazole-6-carboxylic acid

### and

### 2-(((1S,6R)-6-(6-((4-cyano-2-fluorobenzyl)oxy)pyridin-2-yl)-3-azabicyclo[4.1.0]heptan-3-yl)methyl)-1-((1-ethyl-1H-imidazol-5-yl)methyl)-1H-benzo[d]imidazole-6-carboxylic acid

The title compound was prepared and can be prepared similarly following the procedures described by General Procedure K.

### 2-(((1R,6S)-6-(6-((4-cyano-2-fluorobenzyl)oxy)pyridin-2-yl)-3-azabicyclo[4.1.0]heptan-3-yl)methyl)-1-((1-ethyl-1H-imidazol-5-yl)methyl)-1H-benzo[d]imidazole-6-carboxylic acid (Compound 121-P1)

1H NMR (400 MHz, METHANOL-d4) δ 8.11 (s, 1H), 7.99 (dd, J=1.2, 8.4 Hz, 1H), 7.71 (d, J=8.6 Hz, 1H), 7.65 (s, 1H), 7.63-7.50 (m, 4H), 6.83 (d, J=7.4 Hz, 1H), 6.62 (d, J=8.2 Hz, 1H), 6.50 (s, 1H), 5.73 (s, 2H), 5.45 (s, 2H), 4.00 (td, J=7.2, 14.4 Hz, 2H), 3.80 (s, 2H), 2.91 (dd, J=6.4, 11.4 Hz, 1H), 2.70 (br d, J=11.4 Hz, 1H), 2.29 (s, 3H), 1.94-1.84 (m, 1H), 1.69 (br d, J=7.2 Hz, 1H), 1.20 (t, J=7.2 Hz, 3H), 1.03 (dd, J=3.4, 9.0 Hz, 1H), 0.70 (dd, J=3.8, 5.6 Hz, 1H).

### 2-(((1S,6R)-6-(6-((4-cyano-2-fluorobenzyl)oxy)pyridin-2-yl)-3-azabicyclo[4.1.0]heptan-3-yl)methyl)-1-((1-ethyl-1H-imidazol-5-yl)methyl)-1H-benzo[d]imidazole-6-carboxylic acid (Compound 121-P2)

1H NMR (400 MHz, METHANOL-d4) δ 8.11 (s, 1H), 7.99 (dd, J=1.4, 8.4 Hz, 1H), 7.70 (d, J=8.4 Hz, 1H), 7.64 (s, 1H), 7.63-7.50 (m, 4H), 6.83 (d, J=7.4 Hz, 1H), 6.62 (d, J=8.2 Hz, 1H), 6.49 (s, 1H), 5.73 (s, 2H), 5.49-5.41 (m, 2H), 4.00 (quind, J=7.0, 14.5 Hz, 2H), 3.80 (s, 2H), 2.91 (dd, J=6.2, 11.4 Hz, 1H), 2.71 (dd, J=1.4, 11.2 Hz, 1H), 2.33-2.24 (m, 3H), 1.94-1.83 (m, 1H), 1.72-1.64 (m, 1H), 1.20 (t, J=7.2 Hz, 3H), 1.04 (dd, J=3.6, 9.2 Hz, 1H), 0.71 (dd, J=3.6, 6.0 Hz, 1H).

When a mixture of stereoisomers is separated by HPLC, it is to be appreciated that the resultant individual stereoisomers or mixtures will be arbitrarily assigned. In the examples described herein, when the mixture of stereoisomers is separated by HPLC, it is to be appreciated that an eluting enantiomer or an enantiomer of a resulting compound prepared from the eluting enantiomer is labeled “P1” and another eluting enantiomer or an enantiomer of a resulting compound prepared from the another eluting enantiomer is labeled “P2”. In this example, the resulting compound is Compound 121. The absolute configuration of the enantiomers, e.g., Compounds 121-P1 & 121-P2 each associated with the corresponding 1H NMR data, may be obtained by known methods.

### Example 122

### (S)-1-(oxetan-2-ylmethyl)-2-((4-(6-(thiazolo[5,4-b]pyridin-2-ylmethoxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-(thiazolo[5,4-b]pyridin-2-ylmethoxy)pyridin-2-yl)piperazin-1-yl)methyl)-1H-benzo[d]imidazole-6-carboxylic acid (Compound 122)

1H NMR (400 MHz, CHLOROFORM-d) δ 8.58 (dd, J=1.4, 4.6 Hz, 1H), 8.23 (dt, J=1.6, 4.0 Hz, 2H), 8.06 (dd, J=1.4, 8.4 Hz, 1H), 7.82 (d, J=8.4 Hz, 1H), 7.51-7.39 (m, 2H), 6.24 (dd, J=7.8, 19.2 Hz, 2H), 5.73 (s, 2H), 5.24 (dt, J=4.2, 6.6 Hz, 1H), 4.80-4.60 (m, 3H), 4.40 (td, J=5.8, 9.0 Hz, 1H), 4.01 (s, 2H), 3.56-3.43 (m, 4H), 2.72-2.72 (m, 1H), 2.62 (br t, J=4.8 Hz, 4H), 2.52-2.40 (m, 1H).

### Example 123

### (S)-2-((4-(6-((7-fluorobenzo[d]thiazol-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((7-fluorobenzo[d]thiazol-2-yl)methoxy)pyridin-2-yl)piperazin-1-yl)methyl)-1-(oxetan-2-ylmethyl)-1H-benzo[d]imidazole-6-carboxylic acid (Compound 123)

1H NMR (400 MHz, CHLOROFORM-d) δ 8.24 (s, 1H), 8.06 (d, J=8.4 Hz, 1H), 7.88-7.78 (m, 2H), 7.53-7.36 (m, 2H), 7.09 (t, J=8.6 Hz, 1H), 6.26 (d, J=7.8 Hz, 1H), 6.29-6.18 (m, 1H), 5.75 (s, 2H), 5.24 (br d, J=3.8 Hz, 1H), 4.81-4.60 (m, 3H), 4.40 (td, J=5.8, 9.0 Hz, 1H), 4.01 (s, 2H), 3.68-3.44 (m, 4H), 2.95-2.67 (m, 1H), 2.67-2.58 (m, 4H), 2.58-2.31 (m, 2H).

### Example 124 (General Procedure NN)

### (S)-2-((4-(3-((4-cyano-2-fluorobenzyl)oxy)-4-ethyl-1H-pyrazol-1-yl)piperidin-1-yl)methyl)-1-(oxetan-2-ylmethyl)-1H-benzo[d]imidazole-6-carboxylic acid

The title compound was prepared according to Scheme 17. This General Procedure NN exemplifies Scheme 17 and provides particular synthetic details as applied to the title compound.

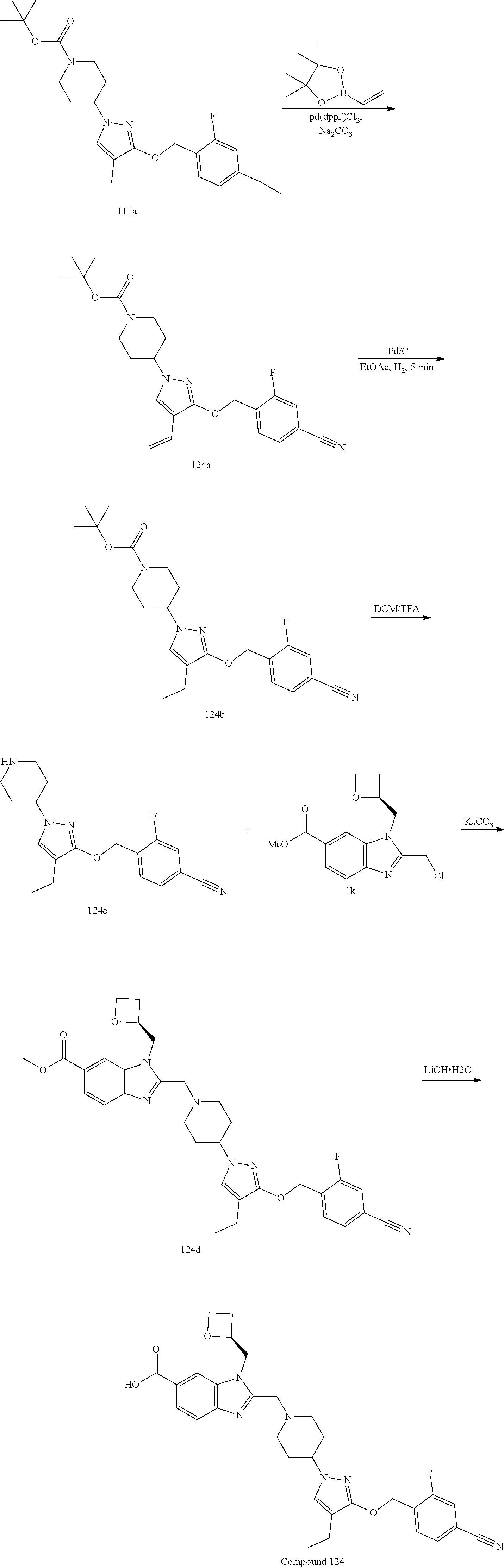

### Tert-butyl 4-(3-((4-cyano-2-fluorobenzyl)oxy)-4-vinyl-1H-pyrazol-1-yl)piperidine-1-carboxylate (124a)

To a mixture of tert-butyl 4-(3-((4-cyano-2-fluorobenzyl)oxy)-4-iodo-1H-pyrazol-1-yl)piperidine-1-carboxylate (111a, 100 mg, 189.99 umol) and 4,4,5,5-tetramethyl-2-vinyl-1,3,2-dioxaborolane (146.31 mg, 949.95 umol, 161.13 uL) in dioxane (5 mL) was added Na2CO3 (2 M, 949.95 uL, 10 eq) and Pd(dppf)Cl₂ (15.52 mg, 19.00 umol) at 20° C. under N2. The mixture was stirred at 80° C. for 16 hours. TLC (Petroleum ether:Ethyl acetate=3:1, Rf=0.52) indicated 111a was consumed completely and one new spot was formed. The reaction mixture was filtered. The filtrate was concentrated under reduced pressure. The residue was purified by prep-TLC (SiO2, Petroleum ether:Ethyl acetate=3:1) to give 124a as a colourless gum. 1H NMR (400 MHz, CDCl3-d) δ 7.68 (t, J=7.6 Hz, 1H), 7.47 (d, J=7.6 Hz, 1H), 7.37 (d, J=9.2 Hz, 1H), 7.03 (s, 1H), 6.50 (q, 1H), 5.55 (q, 3H), 5.09 (d, 1H), 4.20 (br s, 1H), 4.14 (br s, 1H), 4.06-3.95 (m, 1H), 2.87 (br t, J=12.4 Hz, 2H), 2.39 (q, J=7.6 Hz, 2H), 2.04 (br d, J=10.0 Hz, 2H), 1.88-1.73 (m, 2H), 1.48 (s, 9H).

### Tert-butyl 4-(3-((4-cyano-2-fluorobenzyl)oxy)-4-ethyl-1H-pyrazol-1-yl)piperidine-1-carboxylate (124b)

To a solution of tert-butyl 4-(3-((4-cyano-2-fluorobenzyl)oxy)-4-vinyl-1H-pyrazol-1-yl)piperidine-1-carboxylate (124a, 70 mg, 164.13 umol) in Ethyl acetate (1 mL) was added Pd/C (75 mg, 10% purity) under N2 atmosphere. The suspension was degassed and purged with H2 for 3 times. The mixture was stirred under H2 (15 Psi) at 20° C. for 5 minutes. TLC (Petroleum ether:Ethyl acetate=3:1, Rf=0.01) indicated 124a was consumed completely and one new spot was formed. The suspension was filtered through a pad of Celite and the pad cake was washed with Ethyl acetate (10 mL*3). The combined filtrate was concentrated under reduced pressure. The residue was purified by prep-TLC (SiO2, Petroleum ether:Ethyl acetate=3:1) to give 124b as a colourless gum. 1H NMR (400 MHz, CDCl3-d) δ 7.68 (t, J=7.6 Hz, 1H), 7.48 (d, J=7.0 Hz, 1H), 7.37 (dd, J=1.2, 9.2 Hz, 1H), 7.03 (s, 1H), 5.35 (s, 2H), 4.32-4.06 (m, 2H), 4.00 (tt, J=3.8, 11.4 Hz, 1H), 2.86 (br t, J=12.2 Hz, 2H), 2.39 (q, J=7.6 Hz, 2H), 2.11-1.99 (m, 2H), 1.91-1.70 (m, 2H), 1.48 (s, 9H), 1.16 (t, J=7.6 Hz, 3H).

### 4-(((4-ethyl-1-(piperidin-4-yl)-1H-pyrazol-3-yl)oxy)methyl)-3-fluorobenzonitrile (124c)

To a solution of tert-butyl 4-(3-((4-cyano-2-fluorobenzyl)oxy)-4-ethyl-1H-pyrazol-1-yl)piperidine-1-carboxylate (124b, 60 mg, 140.02 umol) in DCM (2 mL) and TFA (0.2 mL) The mixture was stirred at 20° C. for 1 hour. TLC (Petroleum ether:Ethyl acetate=3:1, Rf=0.01) indicated 124b was consumed completely. The reaction mixture was concentrated under reduced pressure to give 124c as a brown gum. 1H NMR (400 MHz, CDCl3-d) δ 7.66 (t, J=7.6 Hz, 1H), 7.48 (d, J=8.0 Hz, 1H), 7.38 (dd, J=1.4, 9.2 Hz, 1H), 7.07 (s, 1H), 5.33 (s, 2H), 4.22 (td, J=4.2, 8.6 Hz, 1H), 3.62 (br s, 2H), 3.23-3.10 (m, 2H), 2.39 (q, J=7.6 Hz, 2H), 2.36-2.24 (m, 2H), 2.24-2.18 (m, 1H), 2.25-2.17 (m, 1H), 1.17 (t, J=7.6 Hz, 3H).

### (S)-methyl 2-((4-(3-((4-cyano-2-fluorobenzyl)oxy)-4-ethyl-1H-pyrazol-1-yl)piperidin-1-yl)methyl)-1-(oxetan-2-ylmethyl)-1H-benzo[d]imidazole-6-carboxylate (124d)

To a solution of 4-(((4-ethyl-1-(piperidin-4-yl)-1H-pyrazol-3-yl)oxy)methyl)-3-fluorobenzonitrile (124c, 60 mg, 182.71 umol) in CH3CN (4 mL) was added K2CO3 (75.76 mg, 548.14 umol). Then (S)-methyl 2-(chloromethyl)-1-(oxetan-2-ylmethyl)-1H-benzo[d]imidazole-6-carboxylate (1k, 53.85 mg, 182.71 umol) was added to the solution and the mixture was stirred at 60° C. for 16 hours. TLC (Ethyl acetate:Methanol=20:1, Rf=0.44) indicated 124c was consumed completely and one new spot was formed. The reaction mixture was extracted with Ethyl acetate (10 mL*3) and H2O (10 mL). The combined organic layers were washed with brine (10 mL), dried over anhydrous Na2SO4, filtered and concentrated under reduced pressure. The residue was purified by prep-TLC (SiO2, Ethyl acetate:Methanol=20:1) to give 124d as a white solid. 1H NMR (400 MHz, CHLOROFORM-d) δ 8.16 (s, 1H), 7.98 (d, J=8.6 Hz, 1H), 7.76 (d, J=8.6 Hz, 1H), 7.67 (t, J=7.2 Hz, 1H), 7.47 (d, J=8.2 Hz, 1H), 7.36 (d, J=8.8 Hz, 1H), 7.04 (s, 1H), 5.34 (s, 2H), 5.22 (br s, 1H), 4.76-4.68 (m, 2H), 4.68-4.59 (m, 1H), 4.42-4.34 (m, 1H), 4.03-3.98 (m, 2H), 3.96 (s, 3H), 3.89 (br s, 1H), 2.99 (br s, 2H), 2.80-2.70 (m, 1H), 2.42-2.26 (m, 4H), 2.05 (s, 2H), 2.01-1.89 (m, 2H), 1.16 (t, J=7.6 Hz, 3H).

### (S)-2-((4-(3-((4-cyano-2-fluorobenzyl)oxy)-4-ethyl-1H-pyrazol-1-yl)piperidin-1-yl)methyl)-1-(oxetan-2-ylmethyl)-1H-benzo[d]imidazole-6-carboxylic acid (Compound 124)

To a solution of (S)-methyl 2-((4-(3-((4-cyano-2-fluorobenzyl)oxy)-4-ethyl-1H-pyrazol-1-yl)piperidin-1-yl)methyl)-1-(oxetan-2-ylmethyl)-1H-benzo[d]imidazole-6-carboxylate (124d, 60 mg, 108.27 umol) in THF (2.1 mL) and H2O (0.9 mL) was added LiOH·H2O (10.30 mg, 245.46 umol, 2.4 eq). The mixture was stirred at 20° C. for 16 hours. TLC (Ethyl acetate:Methanol=20:1, Rf=0.40) indicated 124d was consumed completely and one new spot was formed. LCMS showed 124d was consumed completely and one main peak with desired mass was detected. The reaction mixture was regulated by citric acid to pH=3-4. The reaction mixture was extracted with DCM/i-prOH (10/1, 15 mL*3) and H2O (15 mL). The combined organic layers were washed with brine (15 mL), dried over anhydrous Na2SO4, filtered and concentrated under reduced pressure. The residue was purified by prep-HPLC (column: Waters Xbridge BEH C18 100*30 mm*10 um; mobile phase: [water (10 mM NH4HCO3)-ACN]; B %: 10%-40%, 8 min) to give Compound 124 as a white solid. MS mass calculated for [M+H]+ (C31H33FN6O4) requires m/z 573.25, LCMS found m/z 573.2; 1H NMR (400 MHz, CDCl3-d) δ 8.19 (s, 1H), 8.05 (dd, J=1.6, 8.4 Hz, 1H), 7.81 (d, J=8.6 Hz, 1H), 7.68 (t, J=7.4 Hz, 1H), 7.47 (d, J=8.2 Hz, 1H), 7.36 (dd, J=1.2, 9.4 Hz, 1H), 7.04 (s, 1H), 5.35 (s, 2H), 5.27-5.19 (m, 1H), 4.77-4.61 (m, 3H), 4.40 (td, J=6.0, 9.2 Hz, 1H), 4.02 (s, 2H), 3.96-3.85 (m, 1H), 3.02 (br t, J=11.6 Hz, 2H), 2.81-2.70 (m, 1H), 2.51-2.24 (m, 5H), 2.12-2.03 (m, 2H), 2.02-1.90 (m, 2H), 1.16 (t, J=7.6 Hz, 3H).

### Example 125 (General Procedure OO)

### 2-((4-(6-(benzo[d]thiazol-2-ylmethoxy)pyridin-2-yl)piperidin-1-yl)methyl)-1-((1-ethyl-1H-imidazol-5-yl)methyl)-1H-benzo[d]imidazole-6-carboxylic acid

The title compound was prepared according to Scheme 25. This General Procedure OO exemplifies Scheme 25 and provides particular synthetic details as applied to the title compound.

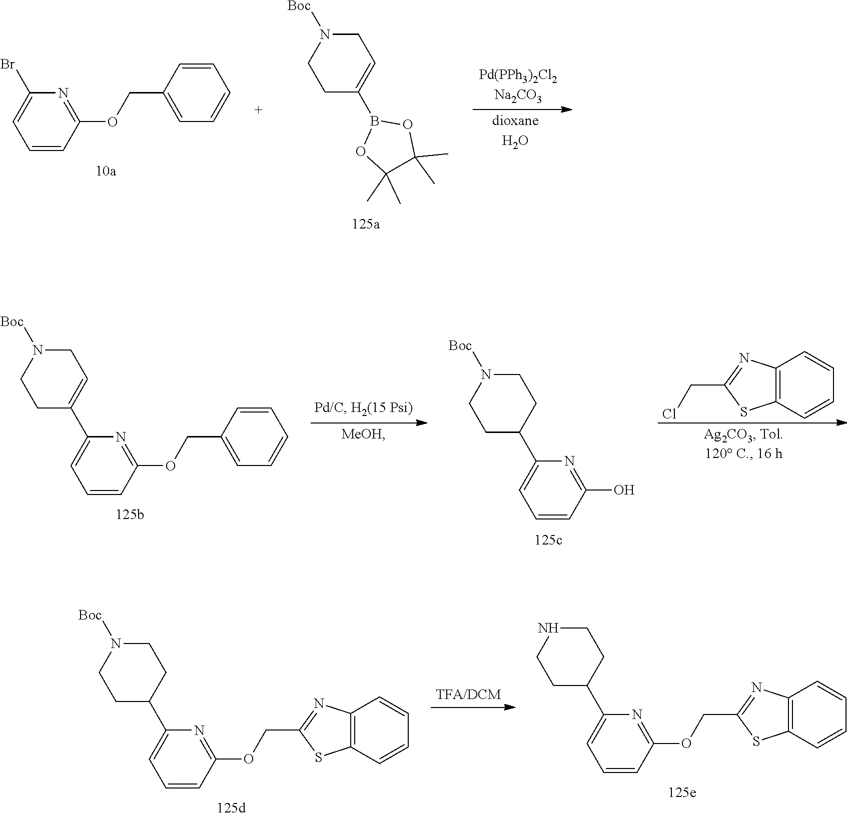

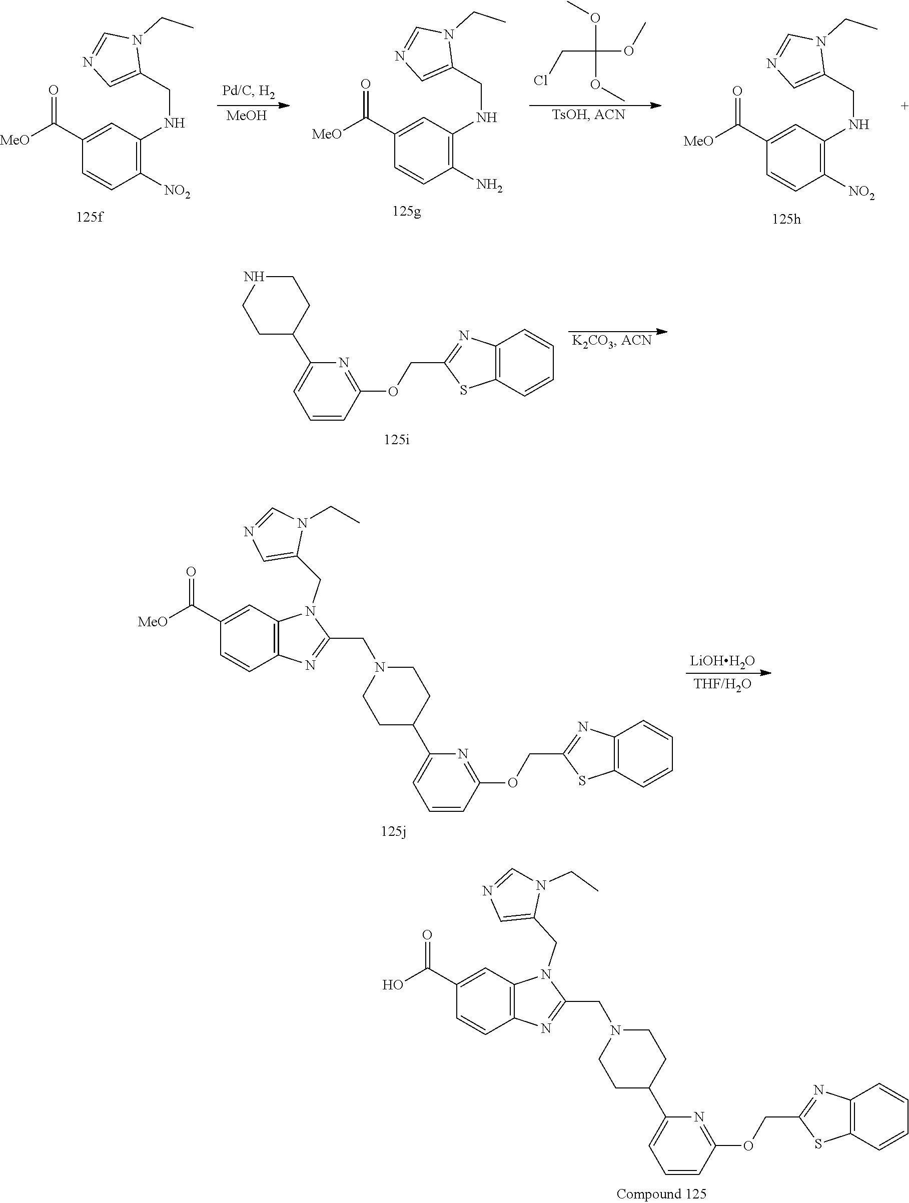

### Tert-butyl 6-(benzyloxy)-5′,6′-dihydro-[2,4′-bipyridine]-1′(2′H)-carboxylate (125b)

To a solution of 2-(benzyloxy)-6-bromopyridine (10a, 5 g, 18.93 mmol) and tert-butyl 4-(4,4,5,5-tetramethyl-1,3,2-dioxaborolan-2-yl)-5,6-dihydropyridine-1(2H)-carboxylate (125a, 7.02 g, 22.72 mmol, 1.2 eq) in dioxane (100 mL) was added H2O (20 mL) and Na2CO3 (6.02 g, 56.79 mmol). The Pd(PPh3)2Cl2 (664.38 mg, 946.55 umol, 0.05 eq) was added in the mixture under N2. Then the mixture was stirred at 100° C. for 16 hours. TLC (Petroleum ether:Ethyl acetate=3:1, Rf=0.6) showed the reaction was completed. The mixture was extracted with Ethyl acetate (100 mL) and H2O (50 mL). The organic layer was dried over Na2SO4, filtered and concentrated in vacuum. The residue was purified by MPLC (SiO2, Petroleum ether:Ethyl acetate=1:0 to 10:1) to give 125b as white oil. 1H NMR (400 MHz, CHLOROFORM-d) δ 7.56 (t, J=7.8 Hz, 1H), 7.47 (d, J=7.4 Hz, 2H), 7.38 (t, J=7.2 Hz, 2H), 7.29-7.35 (m, 1H), 6.95 (d, J=7.4 Hz, 1H), 6.73 (br s, 1H), 6.69 (d, J=8.2 Hz, 1H), 5.42 (s, 2H), 4.11-4.19 (m, 2H), 3.66 (br t, J=5.2 Hz, 2H), 2.62 (br s, 2H), 1.50 (s, 9H).

### Tert-butyl 4-(6-hydroxypyridin-2-yl)piperidine-1-carboxylate (125c)

To a solution of tert-butyl 6-(benzyloxy)-5′,6′-dihydro-[2,4′-bipyridine]-1′(2′H)-carboxylate (125b, 1g, 2.73 mmol) in MeOH (10 mL) was added Pd/C (100 mg, 10% purity) under N2. The suspension was degassed under vacuum and purged with H2 several times. The mixture was stirred under H2 (15 psi) at 20° C. for 16 hours. LCMS showed 125b was consumed completely and one main peak with desired mass was detected. The reaction mixture was filtered and the filtrate was concentrated under reduce pressure to give 125c as a white solid. 1H NMR (400 MHz, CDCl3-d) δ 12.49 (br s, 1H), 7.40 (dd, J=7.2, 9.0 Hz, 1H), 6.42 (d, J=9.0 Hz, 1H), 6.05 (d, J=6.8 Hz, 1H), 4.25 (br s, 2H), 2.96-2.77 (m, 2H), 2.68 (br t, J=12.0 Hz, 1H), 1.95 (br d, J=12.2 Hz, 2H), 1.61-1.55 (m, 2H), 1.48 (s, 9H).

### Tert-butyl 4-(6-(benzo[d]thiazol-2-ylmethoxy)pyridin-2-yl)piperidine-1-carboxylate (125d)

To a solution of tert-butyl 4-(6-hydroxypyridin-2-yl)piperidine-1-carboxylate (125c, 300 mg, 1.08 mmol) in toluene (2 mL) was added 2-(chloromethyl)benzo[d]thiazole (197.95 mg, 1.08 mmol) and Ag2CO3 (594.40 mg, 2.16 mmol, 97.76 uL). The mixture was stirred at 120° C. for 16 hours under N2. LCMS showed 125c was consumed completely and one main peak with desired mass was detected. The reaction mixture was diluted with Ethyl acetate (10 mL). The mixture was filtered and the filter cake was washed with Ethyl acetate (10 mL*2). The combined filtrate was concentrated in vacuu. The residue was purified by column chromatography (SiO2, Petroleum ether:Ethyl acetate=20:1 to 5:1) to give 125d as a yellow solid. 1H NMR (400 MHz, CDCl3-d) δ 8.04 (d, J=8.2 Hz, 1H), 7.88 (d, J=8.0 Hz, 1H), 7.58 (t, J=7.6 Hz, 1H), 7.48 (t, J=7.6 Hz, 1H), 7.40 (t, J=7.6 Hz, 1H), 6.79 (d, J=7.2 Hz, 1H), 6.74 (d, J=8.2 Hz, 1H), 5.83 (s, 2H), 4.11-4.28 (m, 2H), 2.82 (br s, 2H), 2.74 (br s, 1H), 1.88 (br d, J=13.0 Hz, 2H), 1.67-1.76 (m, 2H), 1.49 (s, 9H).

### 2-(((6-(piperidin-4-yl)pyridin-2-yl)oxy)methyl)benzo[d]thiazole (125e)

The solution of tert-butyl 4-(6-(benzo[d]thiazol-2-ylmethoxy)pyridin-2-yl)piperidine-1-carboxylate (125d, 260 mg, 610.98 umol) in TFA (0.2 mL) and DCM (2 mL) was stirred at 20° C. for 1 hour. LCMS showed 125d was consumed completely and one main peak with desired mass was detected. The reaction mixture was concentrated under reduced pressure to give 125e as yellow gum. The residue was used directly for the next step without purification. 1H NMR (400 MHz, MeOD-d4) δ 7.97 (dd, J=4.2, 8.0 Hz, 2H), 7.71 (dd, J=7.4, 8.2 Hz, 1H), 7.53 (t, J=7.6 Hz, 1H), 7.44 (t, J=7.6 Hz, 1H), 6.94 (d, J=7.2 Hz, 1H), 6.84 (d, J=7.8 Hz, 1H), 5.83 (s, 2H), 3.43 (br d, J=13.0 Hz, 2H), 3.09 (dt, J=3.8, 12.6 Hz, 2H), 3.02-2.94 (m, 1H), 2.09-1.93 (m, 4H).

### Methyl 4-amino-3-(((1-ethyl-1H-imidazol-5-yl)methyl)amino)benzoate (125g)

To a solution of methyl 3-(((1-ethyl-1H-imidazol-5-yl)methyl)amino)-4-nitrobenzoate (125f, 430 mg, 1.41 mmol) in MeOH (5 mL) was added Pd/C (50 mg, 10% purity) under N2. The suspension was degassed under vacuum and purged with H2 several times. The mixture was stirred under H2 (15 psi) at 20° C. for 2 hours. LCMS showed 125f was consumed completely and one main peak with desired mass was detected. The reaction mixture was filtered and the filtrate was concentrated under reduce pressure to give 125g as a white solid. 1H NMR (400 MHz, CDCl3-d) δ 7.50-7.58 (m, 2H), 7.49 (s, 1H), 7.04 (s, 1H), 6.72 (d, J=8.0 Hz, 1H), 4.29 (br d, J=4.4 Hz, 2H), 4.03 (q, J=7.2 Hz, 2H), 3.89 (s, 3H), 3.82 (br s, 2H), 3.21 (br s, 1H), 1.47 (t, J=7.2 Hz, 3H).

### Methyl 2-(chloromethyl)-1-((1-ethyl-1H-imidazol-5-yl)methyl)-1H-benzo[d]imidazole-6-carboxylate (125h)

To the solution of methyl 4-amino-3-(((1-ethyl-1H-imidazol-5-yl)methyl)amino)benzoate (125g, 20 mg, 72.91 umol) in ACN (2 mL) was added 2-chloro-1,1,1-trimethoxy-ethane (22.54 mg, 145.82 umol, 19.60 uL) and TsOH (1.26 mg, 7.29 umol). The mixture was stirred at 60° C. for 16 hours. LCMS showed 125g was consumed completely and one main peak with desired mass was detected. The reaction mixture was concentrated under reduced pressure to give 125h as a yellow solid. The product was used directly in next step. MS mass calculated for [M+H]+ (C16H17ClN402) requires m/z 333.1, LCMS found m/z 333.0.

### Methyl 2-((4-(6-(benzo[d]thiazol-2-ylmethoxy)pyridin-2-yl)piperidin-1-yl)methyl)-1-((1-ethyl-1H-imidazol-5-yl)methyl)-1H-benzo[d]imidazole-6-carboxylate (125j)

To a solution of methyl 2-(chloromethyl)-1-((1-ethyl-1H-imidazol-5-yl)methyl)-1H-benzo[d]imidazole-6-carboxylate (125h, 28.16 mg, 86.54 umol, 1.2 eq) in ACN (2 mL) was added K2CO3 (39.87 mg, 288.48 umol) and 2-(((6-(piperidin-4-yl)pyridin-2-yl)oxy)methyl)benzo[d]thiazole (125i, 24 mg, 72.12 umol). The mixture was stirred at 50° C. for 16 hours. LCMS showed 125h was consumed completely and one main peak with desired mass was detected. The reaction mixture was extracted with Ethyl acetate (10 mL*3) and H2O (10 mL). The combined organic layers were washed with brine (5 mL), dried over anhydrous Na2SO4, filtered and concentrated under reduced pressure. The residue was purified by prep-TLC (SiO2, DCM:MeOH=10:1) to give 125j as a light yellow solid. 1H NMR (400 MHz, CDCl3-d) δ 8.08 (d, J=1.0 Hz, 1H), 8.06-7.98 (m, 2H), 7.87 (d, J=8.0 Hz, 1H), 7.78 (d, J=8.2 Hz, 1H), 7.57 (t, J=7.6 Hz, 1H), 7.54-7.46 (m, 2H), 7.42-7.36 (m, 1H), 6.81-6.73 (m, 3H), 5.83 (s, 2H), 5.67 (s, 2H), 3.94 (s, 3H), 3.91 (s, 1H), 3.86 (q, J=7.4 Hz, 2H), 3.78 (s, 2H), 2.89 (br d, J=11.4 Hz, 2H), 2.68-2.59 (m, 1H), 2.28-2.18 (m, 2H), 1.92-1.82 (m, 2H), 1.81-1.70 (m, 2H), 1.21 (t, J=7.2 Hz, 3H).

### 2-((4-(6-(benzo[d]thiazol-2-ylmethoxy)pyridin-2-yl)piperidin-1-yl)methyl)-1-((1-ethyl-1H-imidazol-5-yl)methyl)-1H-benzo[d]imidazole-6-carboxylic acid (125)

To a solution of methyl 2-((4-(6-(benzo[d]thiazol-2-ylmethoxy)pyridin-2-yl)piperidin-1-yl)methyl)-1-((1-ethyl-1H-imidazol-5-yl)methyl)-1H-benzo[d]imidazole-6-carboxylate (125j, 18 mg, 28.95 umol) in THF (1.4 mL) and H2O (0.6 mL) was added LiOH·H2O (3.64 mg, 86.85 umol) at 20° C. The mixture was stirred at 20° C. for 16 hours. LCMS showed 125j was consumed completely and one main peak with desired mass was detected. Citric acid was added in the reaction mixture until pH=3. Then the mixture was filtered and the filtrate was extracted with DCM/i-PrOH (10:1, 10 mL*3). The combined organic layers was washed with brine (5 mL), dried over Na2SO4, filtered and concentrated under reduce pressure to give Compound 125 as white solid. MS mass calculated for [M+H]+ (C33H33N703S) requires m/z 608.2, LCMS found m/z 608.1; 1H NMR (400 MHz, CDCl3-d) δ 8.06-8.00 (m, 3H), 7.86 (d, J=7.8 Hz, 1H), 7.77 (d, J=9.0 Hz, 1H), 7.69 (s, 1H), 7.57 (t, J=7.6 Hz, 1H), 7.48 (t, J=7.2 Hz, 1H), 7.42-7.34 (m, 1H), 7.02 (s, 1H), 6.79 (d, J=7.2 Hz, 1H), 6.74 (d, J=8.2 Hz, 1H), 5.83 (s, 2H), 5.67 (s, 2H), 3.91-3.82 (m, 4H), 2.96 (br d, J=11.8 Hz, 2H), 2.66 (br s, 2H), 2.30 (br t, J=11.2 Hz, 2H), 1.97-1.68 (m, 4H), 1.17 (t, J=7.2 Hz, 3H).

### Example 126

### 2-(((1R,6S)-6-(6-((2,4-dichlorobenzyl)oxy)pyridin-2-yl)-3-azabicyclo[4.1.0]heptan-3-yl)methyl)-1-(((R)-oxetan-2-yl)methyl)-1H-benzo[d]imidazole-6-carboxylic acid

### and

### 2-(((1S,6R)-6-(6-((2,4-dichlorobenzyl)oxy)pyridin-2-yl)-3-azabicyclo[4.1.0]heptan-3-yl)methyl)-1-(((R)-oxetan-2-yl)methyl)-1H-benzo[d]imidazole-6-carboxylic acid

The title compounds were prepared and can be prepared similarly following the procedures described by General Procedure K.

### 2-(((1S,6R)-6-(6-((2,4-dichlorobenzyl)oxy)pyridin-2-yl)-3-azabicyclo[4.1.0]heptan-3-yl)methyl)-1-(((R)-oxetan-2-yl)methyl)-1H-benzo[d]imidazole-6-carboxylic acid (Compound 126-P1)

¹H NMR (400 MHz, MeOD-d4) δ 8.30 (s, 1H), 7.96 (dd, J=1.4, 8.5 Hz, 1H), 7.65 (d, J=8.5 Hz, 1H), 7.57 (t, J=7.8 Hz, 1H), 7.49-7.40 (m, 2H), 7.28 (dd, J=2.0, 8.3 Hz, 1H), 6.91 (d, J=7.5 Hz, 1H), 6.61 (d, J=8.1 Hz, 1H), 5.40 (d, J=3.8 Hz, 2H), 5.20 (dq, J=2.4, 7.2 Hz, 1H), 4.87-4.81 (m, 1H), 4.69 (dd, J=2.4, 15.3 Hz, 1H), 4.63-4.55 (m, 1H), 4.45 (td, J=6.0, 9.2 Hz, 1H), 4.00 (d, J=13.7 Hz, 1H), 3.83 (d, J=13.8 Hz, 1H), 2.95-2.88 (m, 1H), 2.85-2.79 (m, 1H), 2.78-2.69 (m, 1H), 2.62-2.53 (m, 1H), 2.53-2.46 (m, 1H), 2.43 (t, J=6.0 Hz, 2H), 2.12-2.01 (m, 1H), 1.80-1.72 (m, 1H), 1.18 (dd, J=3.6, 9.2 Hz, 1H), 0.95 (dd, J=3.8, 5.8 Hz, 1H).

### 2-(((1R,6S)-6-(6-((2,4-dichlorobenzyl)oxy)pyridin-2-yl)-3-azabicyclo[4.1.0]heptan-3-yl)methyl)-1-(((R)-oxetan-2-yl)methyl)-1H-benzo[d]imidazole-6-carboxylic acid (Compound 126-P2)

¹H NMR (400 MHz, MeOD-d4) δ 8.31 (s, 1H), 7.97 (dd, J=1.4, 8.6 Hz, 1H), 7.67 (d, J=8.6 Hz, 1H), 7.57 (t, J=8.0 Hz, 1H), 7.49-7.40 (m, 2H), 7.28 (dd, J=2.0, 8.4 Hz, 1H), 6.91 (d, J=7.6 Hz, 1H), 6.61 (d, J=8.2 Hz, 1H), 5.45-5.35 (m, 2H), 5.27-5.18 (m, 1H), 4.87-4.81 (m, 1H), 4.68 (dd, J=2.4, 15.4 Hz, 1H), 4.63-4.54 (m, 1H), 4.40 (td, J=6.0, 9.0 Hz, 1H), 4.01-3.87 (m, 2H), 2.99 (dd, J=6.3, 11.6 Hz, 1H), 2.81-2.67 (m, 2H), 2.57 (br dd, J=6.0, 13.6 Hz, 1H), 2.52-2.40 (m, 3H), 2.13-2.02 (m, 1H), 1.75 (q, J=6.8 Hz, 1H), 1.18 (dd, J=3.6, 9.2 Hz, 1H), 0.95 (dd, J=3.8, 5.8 Hz, 1H).

When a mixture of stereoisomers is separated by HPLC, it is to be appreciated that the resultant individual stereoisomers or mixtures will be arbitrarily assigned. In the examples described herein, when the mixture of stereoisomers is separated by HPLC, it is to be appreciated that an eluting enantiomer or an enantiomer of a resulting compound prepared from the eluting enantiomer is labeled “P1” and another eluting enantiomer or an enantiomer of a resulting compound prepared from the another eluting enantiomer is labeled “P2”. In this example, the resulting compound is Compound 126. The absolute configuration of the enantiomers, e.g., Compounds 126-P1 & 126-P2 each associated with the corresponding 1H NMR data, may be obtained by known methods.

### Example 127

### (S)-2-((4-(6-(6-chloro-8-fluoro-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-(6-chloro-8-fluoro-3,4-dihydroisoquinolin-2(1H)-yl)pyridin-2-yl)piperazin-1-yl)methyl)-1-(oxetan-2-ylmethyl)-1H-benzo[d]imidazole-6-carboxylic acid (Compound 127)

1H NMR (400 MHz, METHANOL-d4) δ 8.34 (s, 1H), 7.98 (d, J=8.6 Hz, 1H), 7.68 (d, J=8.4 Hz, 1H), 7.38 (t, J=8.0 Hz, 1H), 7.06-6.97 (m, 2H), 6.18 (d, J=8.0 Hz, 1H), 6.11 (d, J=8.0 Hz, 1H), 5.33-5.23 (m, 1H), 4.93 (br s, 1H), 4.79-4.71 (m, 1H), 4.68-4.56 (m, 3H), 4.47 (td, J=6.0, 9.0 Hz, 1H), 4.10-4.01 (m, 1H), 3.98-3.89 (m, 1H), 3.81 (t, J=5.8 Hz, 2H), 3.57-3.47 (m, 4H), 2.90 (br t, J=5.6 Hz, 2H), 2.85-2.75 (m, 1H), 2.70-2.58 (m, 4H), 2.57-2.48 (m, 1H).

### Example 128

### (S)-2-((4-(6-((6-fluorobenzo[d]thiazol-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((6-fluorobenzo[d]thiazol-2-yl)methoxy)pyridin-2-yl)piperazin-1-yl)methyl)-1-(oxetan-2-ylmethyl)-1H-benzo[d]imidazole-6-carboxylic acid (Compound 128)

1H NMR (400 MHz, CHLOROFORM-d) δ 8.23 (d, J=1.0 Hz, 1H), 8.05 (dd, J=1.4, 8.4 Hz, 1H), 7.94 (dd, J=4.8, 8.8 Hz, 1H), 7.81 (d, J=8.6 Hz, 1H), 7.54 (dd, J=2.4, 8.0 Hz, 1H), 7.46 (t, J=7.8 Hz, 1H), 7.20 (dt, J=2.6, 8.8 Hz, 1H), 6.23 (dd, J=7.8, 13.4 Hz, 2H), 5.71 (s, 2H), 5.24 (br dd, J=3.0, 6.4 Hz, 1H), 4.79-4.60 (m, 3H), 4.40 (td, J=5.8, 9.2 Hz, 1H), 4.01 (s, 2H), 3.50 (br d, J=4.4 Hz, 4H), 2.81-2.70 (m, 1H), 2.62 (br t, J=5.0 Hz, 4H), 2.52-2.40 (m, 1H).

### Example 129

### (S)-2-((4-(6-((5-cyanobenzo[d]thiazol-2-yl)methoxy)pyridin-2-yl)piperazin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((5-cyanobenzo[d]thiazol-2-yl)methoxy)pyridin-2-yl)piperazin-1-yl)methyl)-1-(oxetan-2-ylmethyl)-1H-benzo[d]imidazole-6-carboxylic acid (Compound 129)

1H NMR (400 MHz, CHLOROFORM-d) δ 8.30 (s, 1H), 8.23 (s, 1H), 8.05 (br d, J=8.2 Hz, 1H), 7.97 (d, J=8.2 Hz, 1H), 7.81 (d, J=8.6 Hz, 1H), 7.62 (dd, J=1.2, 8.2 Hz, 1H), 7.48 (t, J=7.8 Hz, 1H), 6.24 (dd, J=8.0, 12.8 Hz, 2H), 5.75 (s, 2H), 5.29-5.20 (m, 1H), 4.78-4.61 (m, 3H), 4.40 (td, J=6.0, 9.0 Hz, 1H), 4.05-3.96 (m, 2H), 3.57-3.43 (m, 4H), 2.82-2.70 (m, 1H), 2.62 (br t, J=4.8 Hz, 4H), 2.52-2.40 (m, 1H).

### Example 130

### (S)-1-(oxetan-2-ylmethyl)-2-((4-(6-(thiazolo[4,5-c]pyridin-2-ylmethoxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-(thiazolo[4,5-c]pyridin-2-ylmethoxy)pyridin-2-yl)piperazin-1-yl)methyl)-1H-benzo[d]imidazole-6-carboxylic acid (Compound 130)

1H NMR (400 MHz, CHLOROFORM-d) δ 9.32 (s, 1H), 8.55 (d, J=5.4 Hz, 1H), 8.22 (s, 1H), 8.06 (d, J=8.6 Hz, 1H), 7.86-7.79 (m, 2H), 7.47 (t, J=7.8 Hz, 1H), 6.23 (dd, J=8.0, 16.0 Hz, 2H), 5.76 (s, 2H), 5.26-5.19 (m, 1H), 4.75-4.59 (m, 3H), 4.39 (td, J=5.8, 9.0 Hz, 1H), 4.04-3.96 (m, 2H), 3.53-3.42 (m, 4H), 2.78-2.68 (m, 1H), 2.62 (br t, J=4.6 Hz, 4H), 2.50-2.39 (m, 1H).

### Example 131

### (S)-1-(oxetan-2-ylmethyl)-2-((4-(6-((4,5,6,7-tetrahydrobenzo[d]thiazol-2-yl)methoxy)pyridin-2-yl)piperazin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-((4,5,6,7-tetrahydrobenzo[d]thiazol-2-yl)methoxy)pyridin-2-yl)piperazin-1-yl)methyl)-1H-benzo[d]imidazole-6-carboxylic acid (Compound 131)

1H NMR (400 MHz, CHLOROFORM-d) δ 8.23 (s, 1H), 8.05 (d, J=8.6 Hz, 1H), 7.81 (d, J=8.6 Hz, 1H), 7.42 (t, J=7.8 Hz, 1H), 6.18 (d, J=7.6 Hz, 2H), 5.55 (s, 2H), 5.28-5.21 (m, 1H), 4.80-4.61 (m, 3H), 4.41 (td, J=6.0, 9.0 Hz, 1H), 4.04 (s, 2H), 3.59-3.48 (m, 4H), 2.81-2.70 (m, 5H), 2.67 (br t, J=4.6 Hz, 4H), 2.52-2.42 (m, 1H), 1.89-1.80 (m, 4H)

### Example 132 (General Procedure PP)

### (S)-2-((4-(3-((4-cyano-2-fluorobenzyl)oxy)-4-cyclopropyl-1H-pyrazol-1-yl)piperidin-1-yl)methyl)-1-(oxetan-2-ylmethyl)-1H-benzo[d]imidazole-6-carboxylic acid

The title compound was prepared according to Scheme 17. This General Procedure PP exemplifies Scheme 17 and provides particular synthetic details as applied to the title compound.

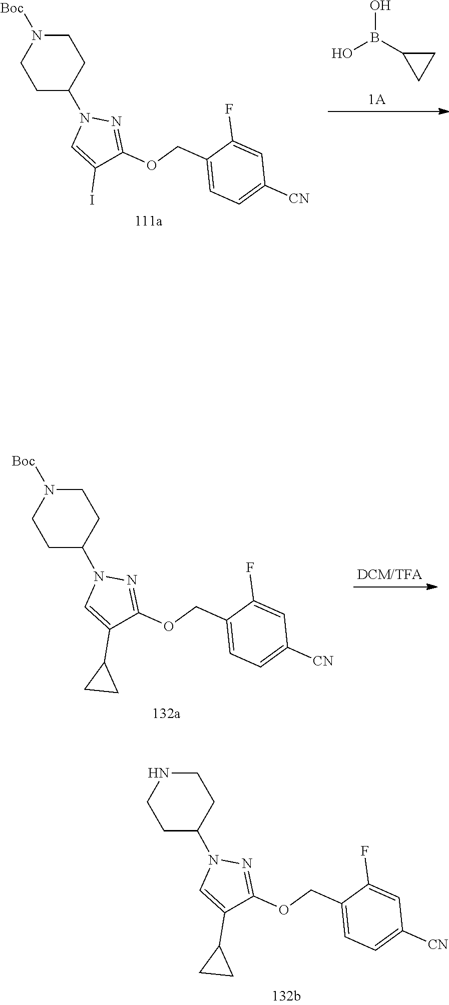 [SMILES: CC(C)(C)OC(=O)N1CCC(n2cc(I)c(OCc3ccc(C#N)cc3F)n2)CC1 ; OB(O)C1CC1 ; CC(C)(C)OC(=O)N1CCC(n2cc(C3CC3)c(OCc3ccc(C#N)cc3F)n2)CC1 ; N#Cc1ccc(COc2nn(C3CCNCC3)cc2C2CC2)c(F)c1]

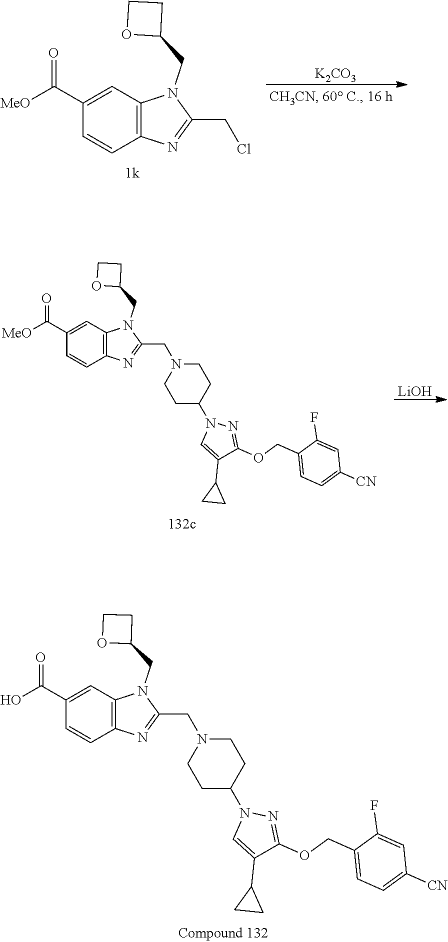 [SMILES: COC(=O)c1ccc2nc(CCl)n(C[C@@H]3CCO3)c2c1 ; "K2CO3" ; COC(=O)c1ccc2nc(CN3CCC(n4cc(C5CC5)c(OCc5ccc(C#N)cc5F)n4)CC3)n(C[C@@H]3CCO3)c2c1 ; "LiOH" ; N#Cc1ccc(COc2nn(C3CCN(Cc4nc5ccc(C(=O)O)cc5n4C[C@@H]4CCO4)CC3)cc2C2CC2)c(F)c1]

### Tert-butyl 4-(3-((4-cyano-2-fluorobenzyl)oxy)-4-cyclopropyl-1H-pyrazol-1-yl)piperidine-1-carboxylate (132a)

To a mixture of tert-butyl 4-(3-((4-cyano-2-fluorobenzyl)oxy)-4-iodo-1H-pyrazol-1-yl)piperidine-1-carboxylate (111a, 100 mg, 189.99 umol) and cyclopropylboronic acid (163.20 mg, 1.90 mmol, 10 eq) in T-AMYL METHACRYLATE (1.5 mL) was added Cs2CO3 (123.81 mg, 379.98 umol, 949.95 uL) and [2-(2-aminophenyl)phenyl]-chloro-palladium;dicyclohexyl-[2-(2,6-dimethoxyphenyl)phenyl]phosphane (13.69 mg, 19.00 umol) under N2. The mixture was degassed under vacuum and purged with N2 several times, and stirred at 80° C. for 16 hours. TLC (Petroleum ether:Ethyl acetate=3:1, Rf=0.50) indicated the starting material was consumed completely and one new spot formed. The residue was poured into water (10 mL) and extracted with ethyl acetate (30 mL*2). The combined organic phase was washed with brine (20 mL*2), dried with anhydrous Na2SO4, filtered and concentrated in vacuum. The residue was purified by prep-TLC (SiO2, Petroleum ether:Ethyl acetate=3:1) to give 132a as a yellow solid. 1H NMR (400 MHz, CDCl3-d) δ 7.71 (q, J=7.8 Hz, 1H), 7.49 (t, J=6.8 Hz, 1H), 7.37 (br d, J=9.4 Hz, 1H), 7.28-7.28 (m, 1H), 5.45-5.26 (m, 2H), 4.36-3.91 (m, 3H), 2.86 (br s, 2H), 2.08-1.97 (m, 2H), 1.88-1.73 (m, 2H), 1.48 (d, J=1.2 Hz, 9H), 1.32-1.19 (m, 2H), 0.87-0.73 (m, 1H), 0.67-0.45 (m, 1H).

### 4-(((4-cyclopropyl-1-(piperidin-4-yl)-1H-pyrazol-3-yl)oxy)methyl)-3-fluorobenzonitrile (132b)

To a mixture of tert-butyl 4-(3-((4-cyano-2-fluorobenzyl)oxy)-4-cyclopropyl-1H-pyrazol-1-yl)piperidine-1-carboxylate (132a, 45 mg, 102.15 umol) in Ethyl acetate (1 mL) was added PTSA (52.77 mg, 306.46 umol) under N2. The mixture was stirred at 20-70° C. for 18 hours. LCMS showed the starting material was consumed completely and desired mass was detected. The residue was poured into NaHCO3 (20 mL) and extracted with ethyl acetate (30 mL*2). The combined organic phase was washed with brine (20 mL*2), dried with anhydrous Na2SO4, filtered and concentrated in vacuum to give 132b as yellow oil. 1H NMR (400 MHz, MeOD-d4) δ 7.75-7.69 (m, 1H), 7.61-7.53 (m, 2H), 7.17 (s, 1H), 5.38-5.29 (m, 2H), 4.18-3.95 (m, 1H), 3.22-3.13 (m, 2H), 2.74 (ddt, J=2.6, 5.4, 12.6 Hz, 2H), 2.06-1.78 (m, 5H), 1.63-1.51 (m, 1H), 1.50-1.44 (m, 1H), 0.79-0.72 (m, 1H), 0.55-0.49 (m, 1H).

### (S)-methyl 2-((4-(3-((4-cyano-2-fluorobenzyl)oxy)-4-cyclopropyl-1H-pyrazol-1-yl)piperidin-1-yl)methyl)-1-(oxetan-2-ylmethyl)-1H-benzo[d]imidazole-6-carboxylate (132c)

To a mixture of 4-(((4-cyclopropyl-1-(piperidin-4-yl)-1H-pyrazol-3-yl)oxy)methyl)-3-fluorobenzonitrile (132b, 35 mg, 102.82 umol) and (S)-methyl 2-(chloromethyl)-1-(oxetan-2-ylmethyl)-1H-benzo[d]imidazole-6-carboxylate (1k, 30.31 mg, 102.82 umol) in CH3CN (3 mL) was added K2CO3 (42.63 mg, 308.47 umol) under N2. The mixture was stirred at 60° C. for 2 hours. TLC (Petroleum ether:Ethyl acetate=0:1, Rf=0.3) indicated the starting material was consumed completely and one new spot formed. The residue was poured into water (15 mL). The aqueous phase was extracted with ethyl acetate (30 mL*2). The combined organic phase was washed with brine (30 mL*2), dried with anhydrous Na2SO4, filtered and concentrated in vacuum. The residue was purified by prep-TLC (SiO2, Petroleum ether:Ethyl acetate=0:1) to give 132c as light yellow solid. 1H NMR (400 MHz, CDCl3-d) δ 8.15 (s, 1H), 7.98 (d, J=8.6 Hz, 1H), 7.81-7.64 (m, 2H), 7.48 (t, J=6.8 Hz, 1H), 7.36 (br d, J=9.2 Hz, 1H), 7.28 (s, 1H), 7.27 (s, 1H), 6.94 (s, 1H), 5.36 (d, J=8.2 Hz, 2H), 5.26-5.16 (m, 1H), 4.78-4.57 (m, 3H), 4.47-4.30 (m, 1H), 4.04-3.81 (m, 6H), 3.07-2.89 (m, 2H), 2.85-2.70 (m, 1H), 2.54-2.38 (m, 1H), 2.38-2.23 (m, 2H), 2.03 (br s, 2H), 1.96-1.82 (m, 2H), 1.82-1.65 (m, 2H), 1.65-1.43 (m, 1H), 0.84-0.74 (m, 1H), 0.59-0.51 (m, 1H).

### (S)-2-((4-(3-((4-cyano-2-fluorobenzyl)oxy)-4-cyclopropyl-1H-pyrazol-1-yl)piperidin-1-yl)methyl)-1-(oxetan-2-ylmethyl)-1H-benzo[d]imidazole-6-carboxylic acid (132)

To a mixture of (S)-methyl 2-((4-(3-((4-cyano-2-fluorobenzyl)oxy)-4-cyclopropyl-1H-pyrazol-1-yl)piperidin-1-yl)methyl)-1-(oxetan-2-ylmethyl)-1H-benzo[d]imidazole-6-carboxylate (132c, 40 mg, 66.82 umol) in THF (1.4 mL) was added LiOH·H2O (5.61 mg, 133.63 umol) in H2O (0.6 mL) under N2. The mixture was stirred at 20° C. for 32 hours. LCMS showed trace of starting material was remained and desired mass was detected. The mixture was quenched by addition citric (10%) to just to pH=6-7, and concentrated under reduced pressure. The residue was purity by HPLC (column: Waters Xbridge BEH C18 100*25 mm*5 um; mobile phase: [water (10 mM NH4HCO3)-ACN]; B %: 15%-50%, 10 min) to give Compound 132 as a white solid. 1H NMR (400 MHz, MeOD-d4) δ 8.33 (s, 1H), 7.98 (br d, J=8.4 Hz, 1H), 7.73 (s, 1H), 7.70-7.64 (m, 1H), 7.63-7.56 (m, 2H), 7.18 (s, 1H), 5.33 (s, 2H), 5.30-5.22 (m, 1H), 4.79-4.69 (m, 1H), 4.65 (br d, J=5.4 Hz, 1H), 4.47 (br d, J=9.2 Hz, 1H), 4.02 (s, 1H), 3.99-3.82 (m, 2H), 3.08-2.90 (m, 2H), 2.88-2.76 (m, 1H), 2.60-2.48 (m, 1H), 2.40-2.26 (m, 2H), 1.98 (br d, J=8.0 Hz, 4H), 1.59-1.51 (m, 1H), 1.31 (s, 1H), 0.75 (br dd, J=2.0, 8.4 Hz, 2H), 0.55-0.47 (m, 2H).

### Example 133

### 2-((4-(6-(6-cyano-8-fluoro-3-methyl-3,4-dihydroisoquinolin-2(1H)-yl)pyridin-2-yl)piperazin-1-yl)methyl)-1-(((S)-oxetan-2-yl)methyl)-1H-benzo[d]imidazole-6-carboxylic acid

The title compound was prepared and can be prepared similarly following the procedures described by General Procedure B.

### 2-((4-(6-(6-cyano-8-fluoro-3-methyl-3,4-dihydroisoquinolin-2(1H)-yl)pyridin-2-yl)piperazin-1-yl)methyl)-1-(((S)-oxetan-2-yl)methyl)-1H-benzo[d]imidazole-6-carboxylic acid (Compound 133)

1H NMR (400 MHz, CHLOROFORM-d) δ 8.24 (d, J=1.0 Hz, 1H), 8.05 (dd, J=1.6, 8.4 Hz, 1H), 7.82 (d, J=8.6 Hz, 1H), 7.40 (t, J=8.2 Hz, 1H), 7.29 (s, 1H), 7.21 (d, J=9.0 Hz, 1H), 6.07 (dd, J=8.0, 15.8 Hz, 2H), 5.31-5.22 (m, 1H), 4.96-4.96 (m, 1H), 5.05-4.94 (m, 1H), 4.82-4.70 (m, 2H), 4.70-4.61 (m, 1H), 4.42 (td, J=5.8, 9.0 Hz, 1H), 4.25 (d, J=18.8 Hz, 1H), 4.04 (s, 2H), 3.62-3.48 (m, 4H), 3.26-3.16 (m, 1H), 2.83-2.64 (m, 6H), 2.55-2.43 (m, 1H), 1.07 (d, J=6.8 Hz, 3H).

### Example 134

### (S)-2-((4-(6-((5-chlorothiazol-2-yl)methoxy)pyridin-2-yl)piperidin-1-yl)methyl)-1-(oxetan-2-ylmethyl)-1H-benzo[d]imidazole-6-carboxylic acid

The title compound was prepared and can be prepared similarly following the procedures described by General Procedure C.

### (S)-2-((4-(6-((5-chlorothiazol-2-yl)methoxy)pyridin-2-yl)piperidin-1-yl)methyl)-1-(oxetan-2-ylmethyl)-1H-benzo[d]imidazole-6-carboxylic acid (Compound 134)

1H NMR (400 MHz, CD30D-d4) δ 8.33 (d, J=1.0 Hz, 1H), 7.97 (dd, J=1.4, 8.4 Hz, 1H), 7.70-7.59 (m, 3H), 6.91 (d, J=7.2 Hz, 1H), 6.70 (d, J=7.8 Hz, 1H), 5.60 (s, 2H), 5.27 (dq, J=2.6, 7.2 Hz, 1H), 4.92-4.87 (m, 1H), 4.78-4.68 (m, 1H), 4.64 (dt, J=5.8, 7.8 Hz, 1H), 4.47 (td, J=6.0, 9.0 Hz, 1H), 4.11 (d, J=13.8 Hz, 1H), 3.99 (d, J=13.8 Hz, 1H), 3.21-3.11 (m, 1H), 3.04 (br d, J=10.8 Hz, 1H), 2.85-2.67 (m, 2H), 2.58-2.48 (m, 1H), 2.48-2.34 (m, 2H), 2.00-1.83 (m, 4H).

### Example 135

### (S)-2-((4-(3-((4-cyano-2-fluorobenzyl)oxy)-4-(difluoromethyl)-1H-pyrazol-1-yl)piperidin-1-yl)methyl)-1-(oxetan-2-ylmethyl)-1H-benzo[d]imidazole-6-carboxylic acid

The title compound was prepared and can be prepared similarly following the procedures described by General Procedure X.

### (S)-2-((4-(3-((4-cyano-2-fluorobenzyl)oxy)-4-(difluoromethyl)-1H-pyrazol-1-yl)piperidin-1-yl)methyl)-1-(oxetan-2-ylmethyl)-1H-benzo[d]imidazole-6-carboxylic acid (Compound 135)

1H NMR (400 MHz, METHANOL-d4) δ 8.34 (d, J=0.7 Hz, 1H), 7.97 (dd, J=1.4, 8.4 Hz, 1H), 7.78-7.65 (m, 3H), 7.62-7.56 (m, 2H), 6.82-6.51 (m, 1H), 5.40 (s, 2H), 5.26 (dq, J=2.6, 7.2 Hz, 1H), 4.73 (dd, J=2.6, 15.2 Hz, 2H), 4.68-4.57 (m, 2H), 4.47 (td, J=6.0, 9.2 Hz, 1H), 4.10-4.01 (m, 2H), 3.98-3.91 (m, 1H), 3.06 (br d, J=11.4 Hz, 1H), 2.96 (br d, J=11.4 Hz, 1H), 2.88-2.77 (m, 1H), 2.51-2.51 (m, 1H), 2.60-2.49 (m, 1H), 2.45-2.30 (m, 2H), 2.09-1.98 (m, 4H).

### Example 136

### (S)-2-((4-(3-((4-cyano-2-fluorobenzyl)oxy)-4-(hydroxymethyl)-1H-pyrazol-1-yl)piperidin-1-yl)methyl)-1-(oxetan-2-ylmethyl)-1H-benzo[d]imidazole-6-carboxylic acid

The title compound was prepared and can be prepared similarly following the procedures described by General Procedure X.

### (S)-2-((4-(3-((4-cyano-2-fluorobenzyl)oxy)-4-(hydroxymethyl)-1H-pyrazol-1-yl)piperidin-1-yl)methyl)-1-(oxetan-2-ylmethyl)-1H-benzo[d]imidazole-6-carboxylic acid (Compound 136)

1H NMR (400 MHz, DMSO-d6) δ 8.22 (d, J=0.8 Hz, 1H), 7.90-7.85 (m, 1H), 7.79 (dd, J=1.4, 8.4 Hz, 1H), 7.75-7.69 (m, 2H), 7.59 (d, J=8.4 Hz, 1H), 7.52 (s, 1H), 5.28 (s, 2H), 5.08 (dq, J=2.8, 7.0 Hz, 1H), 4.77 (dd, J=7.2, 15.2 Hz, 1H), 4.68-4.60 (m, 1H), 4.49 (dt, J=5.8, 7.6 Hz, 1H), 4.37 (td, J=5.8, 9.0 Hz, 1H), 4.20 (s, 2H), 3.99-3.89 (m, 2H), 3.77 (br d, J=13.4 Hz, 1H), 2.96 (br d, J=11.2 Hz, 1H), 2.83 (br d, J=11.2 Hz, 1H), 2.70-2.68 (m, 1H), 2.75-2.65 (m, 1H), 2.46-2.37 (m, 2H), 2.23 (dq, J=9.4, 11.6 Hz, 2H), 1.97-1.76 (m, 4H).

### Example 137 (General Procedure QQ)

### 2-((4-(6-(6-cyano-8-fluoro-3,4-dihydroisoquinolin-2(1H)-yl)pyridin-2-yl)piperidin-1-yl)methyl)-1-((1-ethyl-1H-imidazol-5-yl)methyl)-1H-benzo[d]imidazole-6-carboxylic acid

The title compound was prepared according to Scheme 26. This General Procedure QQ exemplifies Scheme 26 and provides particular synthetic details as applied to the title compound.

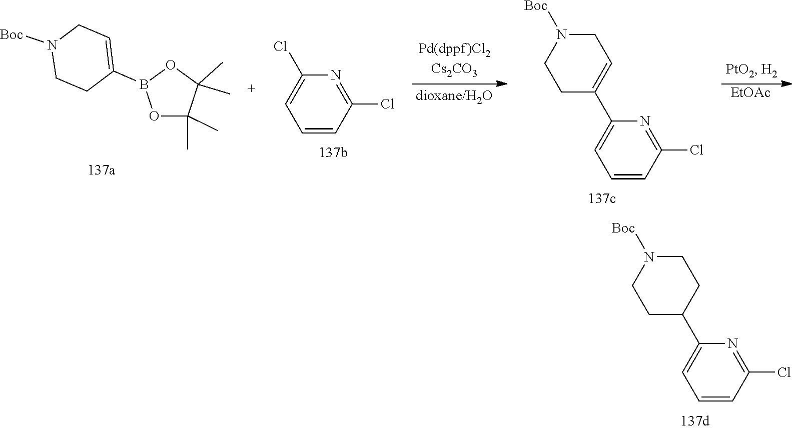 [SMILES: "EtOAc" ; CC(C)(C)OC(=O)N1CC=C(B2OC(C)(C)C(C)(C)O2)CC1 ; Clc1cccc(Cl)n1 ; CC(C)(C)OC(=O)N1CC=C(c2cccc(Cl)n2)CC1 ; CC(C)(C)OC(=O)N1CCC(c2cccc(Cl)n2)CC1]

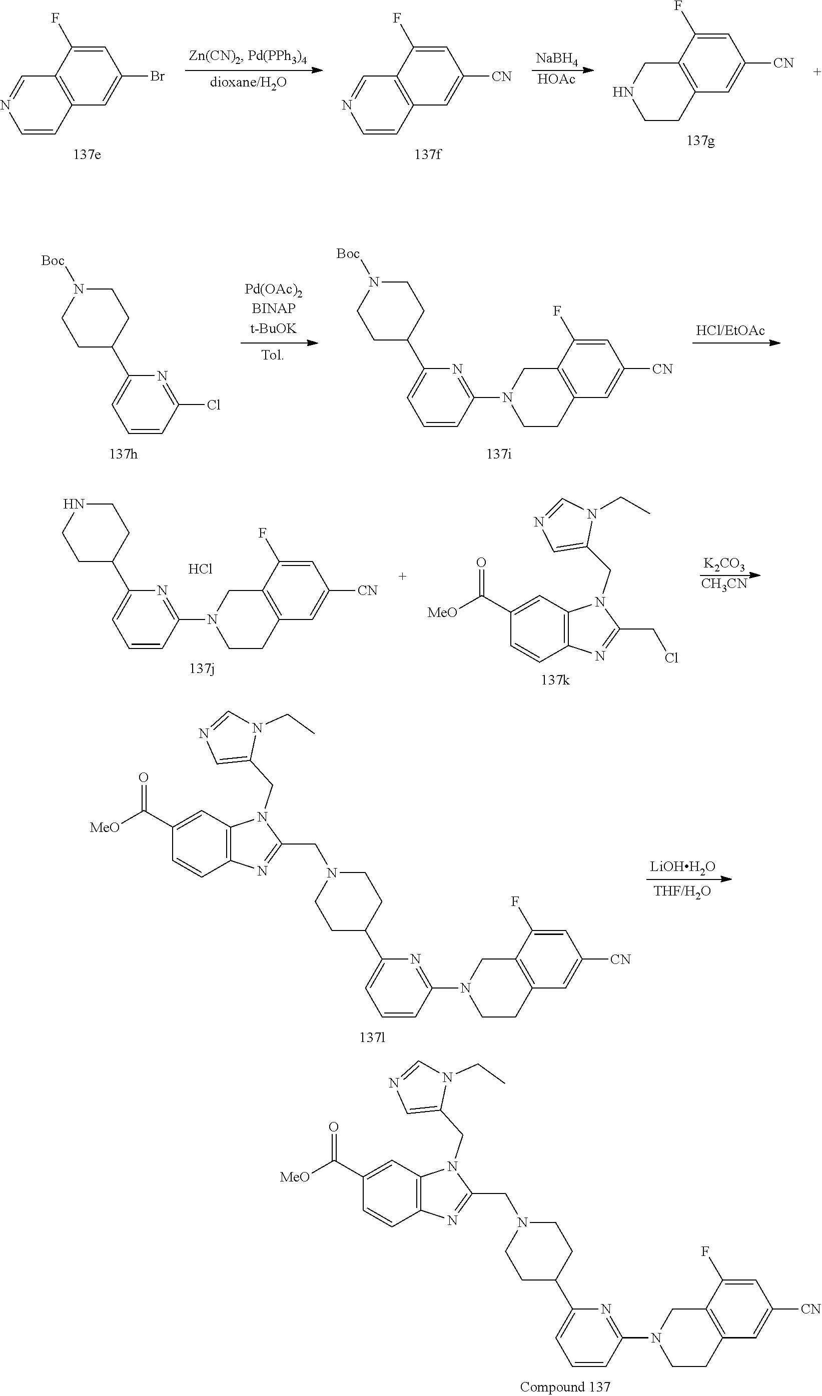

### Tert-butyl 6-chloro-5′,6′-dihydro-[2,4′-bipyridine]-1′(2′H)-carboxylate (137c)

To a solution of 2,6-dichloropyridine (137b, 2 g, 13.51 mmol) and tert-butyl 4-(4,4,5,5-tetramethyl-1,3,2-dioxaborolan-2-yl)-5,6-dihydropyridine-1(2H)-carboxylate (137a, 2.09 g, 6.76 mmol) in dioxane (20 mL) and H2O (4 mL) was added Cs2CO3 (4.84 g, 14.87 mmol). Then Pd(dppf)Cl₂ (494.43 mg, 675.72 umol) was added in the mixture under N2, and the mixture was stirred at 90° C. for 16 hours under N2. LCMS showed most of 137b was consumed and desired mass was detected. The mixture was cooled to 20° C. and extracted with Ethyl acetate (20 mL*2) and H2O (10 mL). The combined organic layers were washed with brine (5 mL), dried over Na2SO4, filtered and concentrated in vacuum. The residue was purified by column silicagel chromatography (Petroleum ether:Ethyl acetate=20:1 to 5:1) to give 137c as colorless oil.

### Tert-butyl 4-(6-chloropyridin-2-yl)piperidine-1-carboxylate (137d)

tert-butyl 6-chloro-5′,6′-dihydro-[2,4′-bipyridine]-1′(2′H)-carboxylate (137c, 100 mg, 339.24 umol) was added to the solution of PtO2 (13.87 mg, 61.06 umol) in Ethyl acetate (2 mL) at 20° C. Then the reaction was stirred at 20° C. for 8 hours under H2 (15 Psi). LCMS detected the desired mass and showed that the 137c was consumed. The mixture was concentrated under reduced pressure. The residue was purified by prep-TLC (Petroleum ether:Ethyl acetate=5:1) to give 137d as colorless oil. 1H NMR (400 MHz, MeOD-d4) δ 7.73 (t, J=7.6 Hz, 1H), 7.26 (dd, J=4.4, 7.6 Hz, 2H), 4.20 (br d, J=13.4 Hz, 2H), 2.96-2.78 (m, 3H), 1.93-1.81 (m, 2H), 1.73-1.61 (m, 2H), 1.75-1.59 (m, 2H), 1.48 (s, 9H).

### 8-fluoroisoquinoline-6-carbonitrile (137f)

Pd(PPh3)4 (51.12 mg, 44.24 umol) and Zn(CN)2 (77.92 mg, 663.58 umol) was added to the solution of 6-bromo-8-fluoroisoquinoline (137e, 100 mg, 442.39 umol) in DMF (1 mL) at 20° C. Then the solution was stirred at 100° C. for 16 hours under N2. LCMS detected the desired mass and showed that 137e was consumed. The reaction mixture was extracted with ethyl acetate (20 mL*2) and the organic layers combined. The resulting mixture was washed with brine (20 mL*2), dried over anhydrous Na2SO4, filtered and concentrated in vacuum. The residue was purified by prep-TLC (Petroleum ether:Ethyl acetate=2:1) to give 137f as a yellow solid. 1H NMR (400 MHz, CDCl3-d) δ 9.64 (s, 1H), 8.80 (d, J=5.8 Hz, 1H), 8.06 (s, 1H), 7.76 (d, J=5.6 Hz, 1H), 7.46-7.40 (m, 1H). 8-fluoro-1,2,3,4-tetrahydroisoquinoline-6-carbonitrile (137g). NaBH4 (13.19 mg, 348.52 umol) was added to the solution of 8-fluoroisoquinoline-6-carbonitrile (137f, 60.00 mg, 348.52 umol) in AcOH (1 mL) at 0° C. The reaction mixture was stirred at 0° C. for 15 minutes. Then NaBH4 (13.19 mg, 348.52 umol) was added to the mixture at 0° C. The solution was stirred at 0° C. for another 15 minutes. LCMS detected the desired mass and showed that 137g was consumed. The mixture was quenched with NH4Cl (10 mL), and extracted with DCM (10 mL*3). The combined organic layer was washed with brine (15 mL), dried over Na2SO4, filtered and concentrated under reduced pressure to give 137g as a yellow solid. 1H NMR (400 MHz, CDCl3-d) δ 7.23 (s, 1H), 7.14 (d, J=9.0 Hz, 1H), 4.07 (s, 2H), 3.14 (t, J=5.8 Hz, 2H), 2.83 (t, J=5.8 Hz, 2H), 2.20 (br s, 1H).

### Tert-butyl 4-(6-(6-cyano-8-fluoro-3,4-dihydroisoquinolin-2(1H)-yl)pyridin-2-yl)piperidine-1-carboxylate (137i)

Pd(OAc)2 (1.27 mg, 5.68 umol), BINAP (10.60 mg, 17.03 umol) and t-BuOK (31.84 mg, 283.78 umol) was added to the solution of tert-butyl 4-(6-chloropyridin-2-yl)piperidine-1-carboxylate (137h, 33.69 mg, 113.51 umol) and 8-fluoro-1,2,3,4-tetrahydroisoquinoline-6-carbonitrile (137g, 20 mg, 113.51 umol) in toluene (1 mL) at 20° C. Then the solution was stirred at 100° C. for 3 hours under N2. LCMS detected the desired mass and showed that 137h was consumed. The mixture was filtered and the filtrate was concentrated under reduced pressure. The residue was purified by prep-TLC (Petroleum ether:Ethyl acetate=5:1) to give 137i as a colorless solid. 1H NMR (400 MHz, MeOD-d4) δ 7.48 (dd, J=7.4, 8.3 Hz, 1H), 7.39-7.30 (m, 2H), 6.68 (d, J=8.4 Hz, 1H), 6.55 (d, J=7.2 Hz, 1H), 4.74 (s, 2H), 4.15 (br d, J=13.2 Hz, 2H), 3.85 (t, J=5.8 Hz, 2H), 2.95 (t, J=5.6 Hz, 2H), 2.91-2.80 (m, 2H), 2.73 (tt, J=3.6, 11.6 Hz, 1H), 1.83 (br d, J=10.8 Hz, 2H), 1.68 (dq, J=4.2, 12.5 Hz, 2H), 1.48 (s, 9H).

### 8-fluoro-2-(6-(piperidin-4-yl)pyridin-2-yl)-1,2,3,4-tetrahydroisoquinoline-6-carbonitrile (137j)

The solution of tert-butyl 4-(6-(6-cyano-8-fluoro-3,4-dihydroisoquinolin-2(1H)-yl)pyridin-2-yl)piperidine-1-carboxylate (137i, 25.2 mg, 57.73 umol) in HCl/EtOAc (0.5 mL) at 20° C. for 10 min. LCMS detected the desired mass and showed that 137i was consumed. The mixture was concentrated to remove the solvent to give 137j as a light yellow solid. 1H NMR (400 MHz, MeOD-d4) δ 8.09 (dd, J=7.4, 9.0 Hz, 1H), 7.57-7.39 (m, 3H), 7.00 (d, J=7.2 Hz, 1H), 5.00 (s, 2H), 4.06 (t, J=5.6 Hz, 2H), 3.62-3.49 (m, 3H), 3.27-3.16 (m, 4H), 2.30 (br d, J=13.8 Hz, 2H), 2.09-1.96 (m, 3H).

### Methyl 2-((4-(6-(6-cyano-8-fluoro-3,4-dihydroisoquinolin-2(1H)-yl)pyridin-2-yl)piperidin-1-yl)methyl)-1-((1-ethyl-1H-imidazol-5-yl)methyl)-1H-benzo[d]imidazole-6-carboxylate (1371)

K2CO3 (39.03 mg, 282.40 umol) was added to the solution of 8-fluoro-2-(6-(piperidin-4-yl)pyridin-2-yl)-1,2,3,4-tetrahydroisoquinoline-6-carbonitrile (137j, 19 mg, 56.48 umol) and methyl methyl 2-(chloromethyl)-1-((1-ethyl-1H-imidazol-5-yl)methyl)-1H-benzo[d]imidazole-6-carboxylate (137k, 30 mg, 54.09 umol, 60% purity) in CH3CN (1 mL) at 20° C. Then the solution was stirred at 50° C. for 3 hours. LCMS detected the desired mass and showed that 137j was consumed. The mixture was filtered and the filtrate concentrated under reduced pressure. The residue was purified by prep-TLC (Ethyl acetate:Methanol=10:1) to give 1371 as a yellow solid. 1H NMR (400 MHz, MeOD-d4) δ 8.14 (s, 1H), 7.98 (dd, J=1.3, 8.6 Hz, 1H), 7.80-7.68 (m, 2H), 7.51-7.32 (m, 3H), 6.69 (d, J=8.4 Hz, 1H), 6.59 (s, 1H), 6.52 (d, J=7.2 Hz, 1H), 5.82 (s, 2H), 4.77 (s, 2H), 4.19-4.07 (m, 2H), 3.94-3.83 (m, 7H), 3.01-2.88 (m, 4H), 2.60-2.49 (m, 1H), 2.24 (br s, 2H), 1.86-1.76 (m, 2H), 1.73-1.60 (m, 2H), 1.35-1.25 (m, 3H).

### 2-((4-(6-(6-cyano-8-fluoro-3,4-dihydroisoquinolin-2(1H)-yl)pyridin-2-yl)piperidin-1-yl)methyl)-1-((1-ethyl-1H-imidazol-5-yl)methyl)-1H-benzo[d]imidazole-6-carboxylic acid (Compound 137)

LiOH·H2O (686.43 ug, 16.36 umol) was added to the solution of methyl 2-((4-(6-(6-cyano-8-fluoro-3,4-dihydroisoquinolin-2(1H)-yl)pyridin-2-yl)piperidin-1-yl)methyl)-1-((1-ethyl-1H-imidazol-5-yl)methyl)-1H-benzo[d]imidazole-6-carboxylate (1371, 6.9 mg, 10.91 umol) in THF (0.7 mL) and H2O (0.3 mL) at 20° C. Then the solution was stirred at 20° C. for 16 hours. LCMS detected the desired mass and showed that 1371 was consumed. The mixture was adjusted to pH=7 with HOAc. The mixture was extracted with Ethyl acetate (10 mL*3). The combined organic layers was washed with brine (15 mL), dried over Na2SO4, filtered and concentrated under reduced pressure. The residue was purified by prep-HPLC (Neutral condition, Waters Xbridge Prep OBD C18 150*40 mm*10 um; mobile phase: [water (10 mM NH4HCO3)-ACN]; B %: 25%-50%, 8 min) to give Compound 137 as a white solid. 1H NMR (400 MHz, MeOD-d4) δ 8.14 (d, J=0.8 Hz, 1H), 8.00 (dd, J=1.4, 8.6 Hz, 1H), 7.80 (s, 1H), 7.71 (d, J=8.6 Hz, 1H), 7.52-7.46 (m, 1H), 7.44-7.35 (m, 2H), 6.70 (d, J=8.4 Hz, 1H), 6.63 (s, 1H), 6.53 (d, J=7.2 Hz, 1H), 5.82 (s, 2H), 4.79 (s, 2H), 4.18-4.09 (m, 2H), 3.93-3.84 (m, 4H), 3.03-2.92 (m, 4H), 2.57 (ddd, J=3.6, 8.2, 11.8 Hz, 1H), 2.27 (br t, J=10.8 Hz, 2H), 1.83 (br d, J=11.0 Hz, 2H), 1.69 (dq, J=3.4, 12.4 Hz, 2H), 1.31 (t, J=7.2 Hz, 3H).

### Example 138 (General Procedure RR)

### 2-((6-(2-(4-chloro-2-fluorophenyl)-2-methylbenzo[d][1,3]dioxol-4-yl)-3-azabicyclo[4.1.0]heptan-3-yl)methyl)-1-((1-ethyl-1H-imidazol-5-yl)methyl)-1H-benzo[d]imidazole-6-carboxylic acid

The title compound was prepared according to Scheme 27. This General Procedure RR exemplifies Scheme 27 and provides particular synthetic details as applied to the title compound.

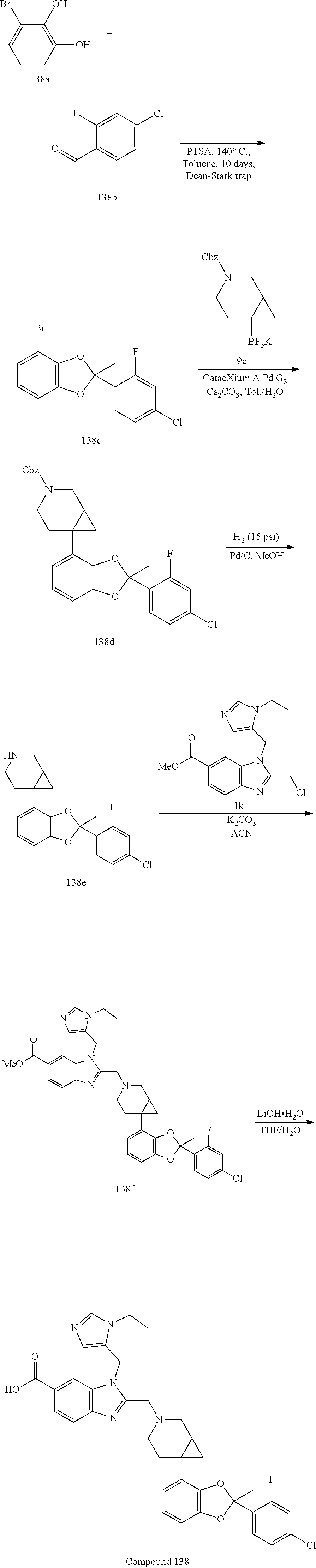

### 4-bromo-2-(4-chloro-2-fluorophenyl)-2-methylbenzo[d][1,3]dioxole (138c)

To a solution of 3-bromobenzene-1,2-diol (138a, 3 g, 15.87 mmol) and 1-(4-chloro-2-fluorophenyl)ethanone (138b, 2.88 g, 16.67 mmol) in toluene (30 mL) was added PTSA (109.33 mg, 634.90 umol). Then the reaction was fitted with a dean-stark trap, and stirred at 140° C. for 24 hours. TLC showed that desired product was formed. The mixture was stirred at 140° C. for another 9 days. The mixture was concentrated in vacuum. The residue was purified by column silica gel chromatography (Petroleum ether:ethyl acetate=5:1) to give the crude product (3.2 g, crude) as a light yellow oil. Then the crude product was diluted in MeOH (50 mL) and the mixture was stirred at 15° C. for 16 hours. The mixture was filtered and the filtrate was concentrated in vacuum to give 138c as light yellow oil. 1H NMR (400 MHz, CDCl3-d): δ 7.50-7.60 (m, 1H), 7.12-7.22 (m, 2H), 6.97 (dd, J=7.8, 1.3 Hz, 1H), 6.66-6.79 (m, 2H), 2.12 (d, J=0.8 Hz, 3H).

### Benzyl 6-(2-(4-chloro-2-fluorophenyl)-2-methylbenzo[d][1,3]dioxol-4-yl)-3-azabicyclo[4.1.0]heptane-3-carboxylate (138d)

To a solution of 4-bromo-2-(4-chloro-2-fluorophenyl)-2-methylbenzo[d][1,3]dioxole (138c, 300 mg, 873.17 umol) in toluene (6 mL) and H2O (0.6 mL) was added [(Z)-(3-benzyloxycarbonyl-3-azabicyclo[4.1.0]heptan-6-yl)boranylidene-fluoranyl]-difluoro-potassium (9c, 294.42 mg, 873.17 umol), CatacXium A Pd G3 (31.80 mg, 43.66 umol) and Cs2CO3 (853.49 mg, 2.62 mmol) under N2. The mixture was stirred at 80° C. for 16 hours under N2. LCMS showed 138c was consumed completely and one main peak with desired mass was detected. The reaction mixture was filtered and the filter cake was washed with Ethyl acetate (10 mL). The mixture was extracted with Ethyl acetate (10 mL*3) and H2O (10 mL). The combined organic layers were washed with brine (5 mL), dried over anhydrous Na2SO4, filtered and concentrated under reduced pressure. The residue was purified by prep-TLC (SiO2, Petroleum ether:ethyl acetate=5:1) to give 138d as light yellow gum. MS mass calculated for [M+H]+ (C28H25ClFNO4) requires m/z 494.1, LCMS found m/z 494.1; 1H NMR (400 MHz, CDCl3-d) δ 7.51 (br s, 1H), 7.37 (br s, 4H), 7.32-7.28 (m, 1H), 7.13 (br s, 2H), 6.80-6.64 (m, 3H), 5.22-5.11 (m, 2H), 3.96 (br d, J=12.0 Hz, 1H), 3.85 (br s, 1H), 3.44 (br s, 2H), 2.15-1.97 (m, 5H), 1.44-1.21 (m, 2H), 1.06 (br s, 1H), 0.79 (br s, 1H).

### 6-(2-(4-chloro-2-fluorophenyl)-2-methylbenzo[d][1,3]dioxol-4-yl)-3-azabicyclo[4.1.0]heptanes (138e)

To a solution of benzyl 6-(2-(4-chloro-2-fluorophenyl)-2-methylbenzo[d][1,3]dioxol-4-yl)-3-azabicyclo[4.1.0]heptane-3-carboxylate (138d, 200 mg, 404.90 umol) in MeOH (2 mL) was added Pd/C (50 mg, 10% purity) under N2. The suspension was degassed under vacuum and purged with H2 several times. The mixture was stirred under H2 (15 psi) at 20° C. for 30 min. TLC indicated 115d was consumed completely and one new spot was formed. The reaction mixture was filtered and the filter cake was concentrated under reduced pressure to give 138e as a white solid. MS mass calculated for [M+H]+ (C20H19ClFNO2) requires m/z 360.1, LCMS found m/z 360.0; 1H NMR (400 MHz, CHLOROFORM-d) δ 7.56 (q, J=8.4 Hz, 1H), 7.18-7.08 (m, 2H), 6.76-6.65 (m, 3H), 3.96 (br d, J=12.0 Hz, 1H), 3.85 (br s, 1H), 3.44 (br s, 2H), 2.15-1.97 (m, 5H), 1.44-1.21 (m, 2H), 1.06 (br s, 1H), 0.79 (br s, 1H).

### Methyl 2-((6-(2-(4-chloro-2-fluorophenyl)-2-methylbenzo[d][1,3]dioxol-4-yl)-3-azabicyclo[4.1.0]heptan-3-yl)methyl)-1-((1-ethyl-1H-imidazol-5-yl)methyl)-1H-benzo[d]imidazole-6-carboxylate (138f)

To a solution of 6-(2-(4-chloro-2-fluorophenyl)-2-methylbenzo[d][1,3]dioxol-4-yl)-3-azabicyclo[4.1.0]heptane (138e, 120 mg, 333.50 umol,) in ACN (2 mL) was added K2CO3 (184.37 mg, 1.33 mmol) and methyl 2-(chloromethyl)-1-((1-ethyl-1H-imidazol-5-yl)methyl)-1H-benzo[d]imidazole-6-carboxylate (1k, 158.55 mg, 333.50 umol, 70% purity). The mixture was stirred at 50° C. for 16 hours. LCMS showed 1k was consumed completely and one main peak with desired mass was detected. The reaction mixture was extracted with Ethyl acetate (10 mL*3) and H2O (10 mL). The combined organic layers were washed with brine (5 mL), dried over anhydrous Na2SO4, filtered and concentrated under reduced pressure. The residue was purified by prep-TLC (SiO2, DCM:MeOH=10:1) to give 138f as a light yellow solid. MS mass calculated for [M+H]+ (C36H35C1FN5O4) requires m/z 656.2, LCMS found m/z 656.2; 1H NMR (400 MHz, CHLOROFORM-d) δ 8.10 (s, 1H), 8.00 (d, J=8.2 Hz, 1H), 7.78 (d, J=8.4 Hz, 1H), 7.57-7.48 (m, 2H), 7.26-7.08 (m, 2H), 6.86 (d, J=3.0 Hz, 1H), 6.76-6.62 (m, 3H), 5.74-5.63 (m, 2H), 3.95 (s, 3H), 3.83 (q, J=7.2 Hz, 2H), 3.74 (s, 2H), 2.92-2.79 (m, 2H), 2.46-2.36 (m, 1H), 2.31 (br d, J=4.8 Hz, 1H), 2.12-2.03 (m, 4H), 2.03-1.94 (m, 1H), 1.43-1.34 (m, 1H), 1.32-1.18 (m, 4H), 1.10-1.01 (m, 1H), 0.91-0.75 (m, 1H).

### 2-((6-(2-(4-chloro-2-fluorophenyl)-2-methylbenzo[d][1,3]dioxol-4-yl)-3-azabicyclo[4.1.0]heptan-3-yl)methyl)-1-((1-ethyl-1H-imidazol-5-yl)methyl)-1H-benzo[d]imidazole-6-carboxylic acid (138)

To a solution of methyl 2-((6-(2-(4-chloro-2-fluorophenyl)-2-methylbenzo[d][1,3]dioxol-4-yl)-3-azabicyclo[4.1.0]heptan-3-yl)methyl)-1-((1-ethyl-1H-imidazol-5-yl)methyl)-1H-benzo[d]imidazole-6-carboxylate (138f, 40 mg, 60.96 umol) in THF (1.4 mL) and H2O (0.6 mL) was added LiOH·H2O (5.12 mg, 121.92 umol). The mixture was stirred at 20° C. for 16 hours. LCMS showed 115f was consumed completely and one main peak with desired mass was detected. To the reaction mixture was added citric acid until pH=4. The mixture was filtered and the filtrate was concentrated under reduced pressure. The mixture was extracted with DCM/i-PrOH (10:1, 10 mL*3). The combined organic layer was washed with brine (5 mL), dried over Na2SO4, filtered and concentrated under reduced pressure. The residue was purified by prep-HPLC (column: Waters Xbridge BEH C18 100*30 mm*10 um; mobile phase: [ ]; B %: 33%-63%, 6 min) to give Compound 138 as a white solid. MS mass calculated for [M+H]+ (C35H33C1FN5O4) requires m/z 642.2, LCMS found m/z 642.1; 1H NMR (400 MHz, MeOD-d4) δ 8.14 (s, 1H), 8.00 (dd, J=1.2, 8.6 Hz, 1H), 7.83 (s, 1H), 7.72 (d, J=8.6 Hz, 1H), 7.58 (t, J=8.2 Hz, 1H), 7.28 (dd, J=1.8, 10.8 Hz, 1H), 7.23-7.18 (m, 1H), 6.74-6.56 (m, 4H), 5.81 (d, J=9.4 Hz, 2H), 4.08 (q, J=7.2 Hz, 2H), 3.89-3.75 (m, 2H), 2.93-2.81 (m, 2H), 2.40 (br s, 1H), 2.37-2.28 (m, 1H), 2.03 (s, 3H), 2.00-1.77 (m, 2H), 1.28 (dt, J=2.4, 7.3 Hz, 4H), 0.99-0.92 (m, 1H), 0.64-0.58 (m, 1H).

### Example 139

### (S)-1-(oxetan-2-ylmethyl)-2-((4-(6-(thiazol-2-ylmethoxy)pyridin-2-yl)piperidin-1-yl)methyl)-1H-benzo[d]imidazole-6-carboxylic acid

The title compound was prepared and can be prepared similarly following the procedures described by General Procedure C.

### (S)-1-(oxetan-2-ylmethyl)-2-((4-(6-(thiazol-2-ylmethoxy)pyridin-2-yl)piperidin-1-yl)methyl)-1H-benzo[d]imidazole-6-carboxylic acid (Compound 139)

1H NMR (400 MHz, METHANOL-d4) δ 8.33 (s, 1H), 7.97 (dd, J=1.4, 8.4 Hz, 1H), 7.77 (d, J=3.2 Hz, 1H), 7.71-7.55 (m, 3H), 6.89 (d, J=7.2 Hz, 1H), 6.70 (d, J=8.2 Hz, 1H), 5.80-5.61 (m, 2H), 5.27 (br d, J=7.0 Hz, 1H), 4.77-4.58 (m, 2H), 4.47 (td, J=5.8, 9.0 Hz, 1H), 4.11 (d, J=13.8 Hz, 1H), 4.00 (d, J=13.8 Hz, 1H), 3.41-3.34 (m, 1H), 3.28-3.10 (m, 1H), 3.04 (br d, J=11.6 Hz, 1H), 2.85-2.62 (m, 2H), 2.58-2.33 (m, 3H), 2.00-1.79 (m, 4H).

### Example 140 (General Procedure SS)

### (S)-2-((4-(6-((4-cyano-2-fluorobenzyl)oxy)pyridin-2-yl)piperazin-1-yl)methyl)-1-((1,1-dioxidothietan-2-yl)methyl)-1H-benzo[d]imidazole-6-carboxylic acid

### and

### (R)-2-((4-(6-((4-cyano-2-fluorobenzyl)oxy)pyridin-2-yl)piperazin-1-yl)methyl)-1-((1,1-dioxidothietan-2-yl)methyl)-1H-benzo[d]imidazole-6-carboxylic acid

The title compound was prepared according to Scheme 25. This General Procedure SS exemplifies Scheme 25 and provides particular synthetic details as applied to the title compound.

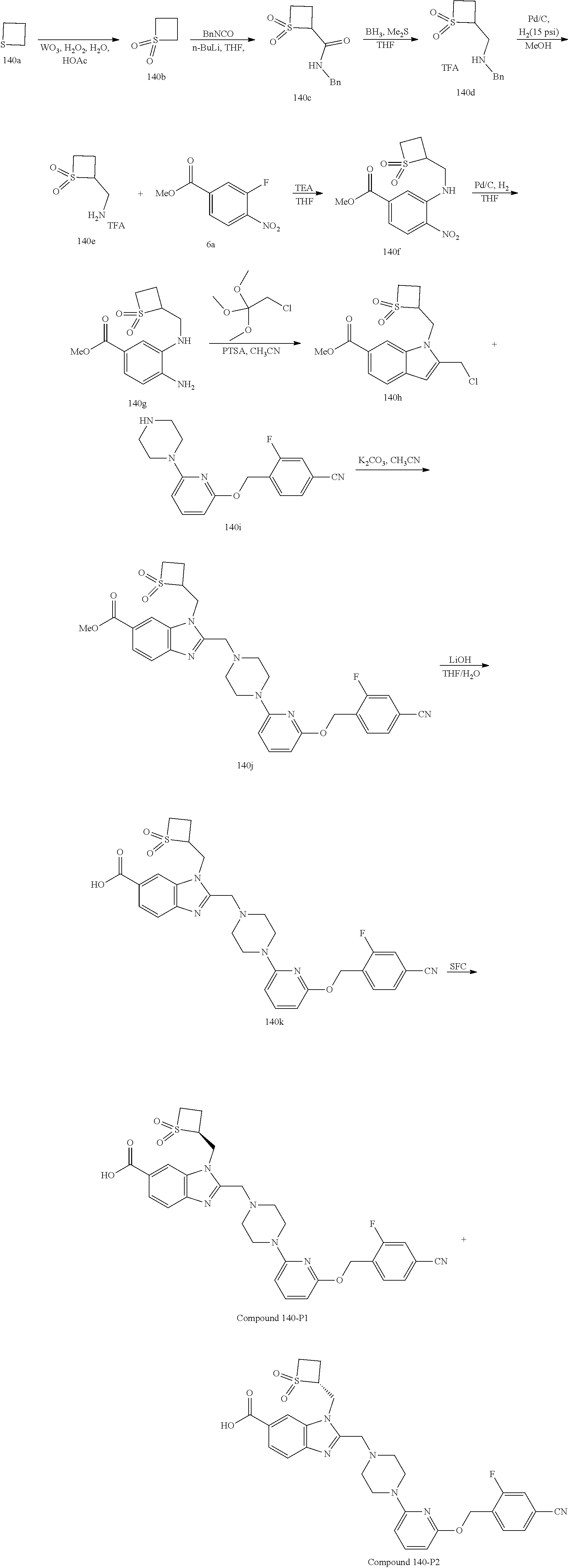

Thietane 1,1-dioxide (140b). The pH of a solution of W03 (218.88 mg, 944.10 umol, 0.07 eq) in H2O (1 mL) is adjusted to 11.5 by addition of NaOH solution (2.5 M, 377.64 uL, 0.07 eq); the white suspension of the tungstate catalyst is added to around-bottomed flask fitted with a magnetic stirrer and a pressure-equalizing addition funnel. The tungstic acid-water mixture is cooled to 0-10° C. by means of an ice-salt bath; AcOH (1 mL) and thietane (140a, 1g, 13.49 mmol) are added in the mixture. The chilled mixture wass stirred, and 30% H2O2 (3.06 g, 26.97 mmol, 2.59 mL, 30% purity) is added carefully by means of the addition funnel over a period of 2 hours. The mixture was stirred at 15° C. for 16 hours. LCMS showed that desired mass (M+23) was found. The mixture was transferred to a beaker, and heated to near dryness on a steam bath. The resulting solid material is triturated five times with 10 mL portions of hot chloroform; any catalyst is removed by filtration. The chloroform solutions are combined and dried over anhydrous magnesium sulfate and the solvent is removed via a rotary evaporator to give 140b as a white solid. 1H NMR (400 MHz, CD3Cl-d) δ 4.19-4.10 (m, 4H), 2.25-2.10 (m, 2H).

### N-benzylthietane-2-carboxamide 1,1-dioxide (140c)

To a mixture of thietane 1,1-dioxide (140b, 600 mg, 5.65 mmol) in THF (5 mL) was added hexyllithium (2.2 M, 2.83 mL) at −78° C., the mixture was stirred at −78° C. for 10 min, then BnNCO (417.68 mg, 5.65 mmol) was added to the mixture dropwise at −78° C. under N2. The mixture was stirred at −78° C. for 1 hour. LCMS showed the starting material was consumed completely and desired mass was detected. The reaction was poured into NH4Cl (5 mL) and concentrated under reduced pressure. The residue was diluted with Ethyl acetate (8 mL), and stirred for 0.5 hours, then the reaction mixture was filtered and concentrated under reduced pressure. The residue was purified by prep-HPLC (column: Phenomenex luna C18 250*50 mm*10 um; mobile phase: [water (0.1% TFA)-ACN]; B %: 10%-50%, 10 min) to give 140c as a white solid. 1H NMR (400 MHz, CD3Cl-d) δ 7.39-7.29 (m, 5H), 7.29-7.21 (m, 1H), 6.55 (br s, 1H), 5.03-4.83 (m, 1H), 4.65-4.42 (m, 2H), 4.29-4.04 (m, 2H), 2.75-2.54 (m, 1H), 2.34 (dtd, J=6.6, 10.0, 12.0 Hz, 1H).

### 2-((benzylamino)methyl)thietane 1,1-dioxide (140d)

To a mixture of N-benzylthietane-2-carboxamide 1,1-dioxide (140c, 300 mg, 1.25 mmol) in THF (3 mL) was added BH3-Me2S (10 M, 626.85 uL) at 0° C. under N2. The mixture was stirred at 75° C. for 2 hours. LCMS showed the starting material was consumed completely and desired mass was detected. The reaction mixture was quenched by addition MeOH (5 mL) at 20° C., then the mixture was stirred for 2 hours at 20° C. The mixture was concentrated under reduced pressure. The residue was purified by prep-HPLC (column: Phenomenex Luna C18 100*30 mm*5 um; mobile phase: [water (0.1% TFA)-ACN]; B %: 5%-30%, 10 min) to give 140d as a white solid. 1H NMR (400 MHz, MeOD-d4) δ 7.59-7.37 (m, 5H), 4.79-4.63 (m, 1H), 4.35-4.16 (m, 3H), 4.16-3.98 (m, 1H), 3.69 (dd, J=7.8, 13.8 Hz, 1H), 3.49 (dd, J=5.4, 14.0 Hz, 1H), 2.47 (dtd, J=4.0, 10.1, 12.0 Hz, 1H), 1.91 (tdd, J=8.6, 10.6, 12.0 Hz, 1H).

### 2-(aminomethyl)thietane 1,1-dioxide (140e)

To a solution of 2-((benzylamino)methyl)thietane 1,1-dioxide (140d, 300 mg, 884.10 umol, TFA) in MeOH (1 mL) was added Pd/C (300 mg, 600.00 umol, 10% purity). The suspension was degassed under vacuum and purged with H2 several times. The mixture was stirred at 20° C. for 2 hours. TLC (Ethyl acetate:Methanol=10:1) indicated the starting material was consumed completely and one new spot was formed. The reaction mixture was filtered and the filtrate was concentrated under reduced pressure to give 140e as colorless oil. 1H NMR (400 MHz, MeOD-d4) δ 4.68-4.56 (m, 1H), 4.22-4.12 (m, 2H), 3.53 (dd, J=8.2, 13.8 Hz, 1H), 3.40-3.33 (m, 1H), 2.43 (dtd, J=4.6, 10.8, 12.0 Hz, 1H), 1.90 (tdd, J=8.2, 10.8, 12.0 Hz, 1H).

### Methyl 3-(((1,1-dioxidothietan-2-yl)methyl)amino)-4-nitrobenzoate (140f)

To a mixture of 2-(aminomethyl)thietane 1,1-dioxide (140e, 220 mg, 1.63 mmol, TFA) and methyl 3-fluoro-4-nitrobenzoate (6a, 388.89 mg, 1.95 mmol) in THF (3 mL) was added TEA (494.03 mg, 4.88 mmol, 679.55 uL) under N2. The mixture was stirred at 75° C. for 16 hours. LCMS showed the starting material was remained and desired mass was detected. The mixture was stirred at 75° C. for another 16 hours. TLC (Petroleum ether:Ethyl acetate=1:1) indicated the most of starting material was consumed and one new spot was formed. The reaction mixture was concentrated under reduced pressure. The residue was purified by column chromatography (SiO2, Petroleum ether:Ethyl acetate=15:1 to 2:1) to give 140f as a yellow solid. 1H NMR (400 MHz, CDCl3-d) δ 8.29-8.17 (m, 2H), 7.56 (d, J=1.4 Hz, 1H), 7.35 (dd, J=1.6, 8.8 Hz, 1H), 4.80-4.61 (m, 1H), 4.21-4.02 (m, 3H), 3.97 (s, 3H), 3.86 (td, J=5.6, 14.4 Hz, 1H), 2.42 (dtd, J=5.0, 9.8, 12.0 Hz, 1H), 2.10-1.88 (m, 1H).

### Methyl 4-amino-3-(((1,1-dioxidothietan-2-yl)methyl)amino)benzoate (140g)

To a solution of methyl 3-(((1,1-dioxidothietan-2-yl)methyl)amino)-4-nitrobenzoate (140f, 150 mg, 477.23 umol) in THF (2 mL) was added Pd/C (150.00 mg, 300.00 umol, 10% purity) under N2. The suspension was degassed under vacuum and purged with H2 several times. The mixture was stirred at 20° C. for 2 hours. LCMS showed the starting material was consumed completely and desired mass was detected. The reaction mixture was filtered and the filtrate was concentrated under reduced pressure to give 140g as alight yellow solid. 1H NMR (400 MHz, MeOD-d4) δ 7.37 (dd, J=1.8, 8.4 Hz, 1H), 7.27 (d, J=1.8 Hz, 1H), 6.67 (d, J=8.2 Hz, 1H), 4.78-4.62 (m, 1H), 4.16-3.91 (m, 2H), 3.82 (s, 3H), 3.75 (dd, J=9.0, 13.8 Hz, 1H), 3.50 (dd, J=5.2, 13.8 Hz, 1H), 2.36 (dtd, J=4.6, 10.0, 11.8 Hz, 1H), 2.48 (dtd, J=4.6, 10.0, 11.8 Hz, 1H), 2.02-1.82 (m, 1H).

### Methyl 2-(chloromethyl)-1-((1,1-dioxidothietan-2-yl)methyl)-1H-benzo[d]imidazole-6-carboxylate (140h)

To a mixture of methyl 4-amino-3-(((1,1-dioxidothietan-2-yl)methyl)amino)benzoate (140g, 120 mg, 422.04 umol) and 2-chloro-1,1,1-trimethoxy-ethane (94.60 mg, 611.96 umol, 82.26 uL) in CH3CN (5 mL) under N2. The mixture was added PTSA (7.27 mg, 42.20 umol). The reaction mixture was stirred at 60° C. for 6 hours. LCMS showed the starting material was consumed and desired mass was detected. The reaction mixture was poured into water (15 mL) and extracted with ethyl acetate (30 mL*2). The combined organic phase was washed with brine (30 mL), dried with anhydrous Na2SO4, filtered and concentrated in vacuum. The residue was purified by prep-TLC (SiO2, Petroleum ether:Ethyl acetate=0:1) to give 140h as a light yellow solid. 1H NMR (400 MHz, MeOD-d4) δ 8.45 (s, 1H), 8.03 (dd, J=1.4, 8.6 Hz, 1H), 7.74 (d, J=8.6 Hz, 1H), 5.26 (d, J=12.8 Hz, 1H), 5.12-4.93 (m, 3H), 4.87-4.74 (m, 1H), 4.22-3.99 (m, 2H), 3.96 (s, 3H), 2.12-1.92 (m, 1H).

### Methyl 2-((4-(6-((4-cyano-2-fluorobenzyl)oxy)pyridin-2-yl)piperazin-1-yl)methyl)-1-((1,1-dioxidothietan-2-yl)methyl)-1H-benzo[d]imidazole-6-carboxylate (140j)

To a mixture of methyl 2-(chloromethyl)-1-((1,1-dioxidothietan-2-yl)methyl)-1H-benzo[d]imidazole-6-carboxylate (140h, 50 mg, 145.86 umol) and 3-fluoro-4-(((6-(piperazin-1-yl)pyridin-2-yl)oxy)methyl)benzonitrile (140i, 45.56 mg, 106.85 umol, TFA) in CH3CN (4 mL) was added K2CO3 (60.48 mg, 437.58 umol) under N2. The mixture was stirred at 55° C. for 16 hours. LCMS showed the starting material was consumed completely and desired mass was detected. The reaction mixture was poured into water (20 mL) and extracted with ethyl acetate (30 mL*2). The combined organic phase was washed with brine (20 mL*2), dried with anhydrous Na2SO4, filtered and concentrated in vacuum. The residue was purified by prep-TLC (SiO2, Petroleum ether:Ethyl acetate=0:1) to give 140j as a white solid 1H NMR (400 MHz, METHANOL-d4) δ 8.43 (s, 1H), 8.00 (d, J=7.2 Hz, 1H), 7.71 (d, J=8.6 Hz, 1H), 7.63 (t, J=7.6 Hz, 1H), 7.59-7.50 (m, 2H), 7.45 (t, J=8.0 Hz, 1H), 6.32-6.27 (m, 1H), 6.16 (br t, J=7.8 Hz, 1H), 5.44 (s, 2H), 5.22-5.13 (m, 2H), 4.18-4.09 (m, 2H), 3.98-3.93 (m, 4H), 3.80 (d, J=13.8 Hz, 1H), 3.49 (br t, J=4.6 Hz, 4H), 2.67-2.53 (m, 4H), 2.44 (q, J=8.8 Hz, 1H), 2.14-2.04 (m, 1H).

### 2-((4-(6-((4-cyano-2-fluorobenzyl)oxy)pyridin-2-yl)piperazin-1-yl)methyl)-1-((1,1-dioxidothietan-2-yl)methyl)-1H-benzo[d]imidazole-6-carboxylic acid (140k)

To a mixture of methyl 2-((4-(6-((4-cyano-2-fluorobenzyl)oxy)pyridin-2-yl)piperazin-1-yl)methyl)-1-((1,1-dioxidothietan-2-yl)methyl)-1H-benzo[d]imidazole-6-carboxylate (140j, 90 mg, 145.47 umol) in THF (3.5 mL) was added LiOH·H2O (12.21 mg, 290.94 umol) in H2O (1.5 mL) under N2. The mixture was stirred at 20° C. for 16 hours. LCMS showed the starting material was consumed completely and desired mass was detected. TLC indicated the starting material was consumed completely and one new spot was formed. The mixture was quenched by addition citric (10%) to just to pH=5˜6, and the reaction mixture were concentrated under reduced pressure. The residue was purified by prep-TLC (SiO2, DCM:MeOH=10:1) to give 140k as a white solid. 1H NMR (400 MHz, MeOD-d4) δ 8.34 (s, 1H), 7.99 (d, J=8.6 Hz, 1H), 7.69-7.59 (m, 2H), 7.59-7.50 (m, 2H), 7.45 (t, J=7.8 Hz, 1H), 6.29 (d, J=8.0 Hz, 1H), 6.14 (d, J=7.8 Hz, 1H), 5.43 (s, 2H), 5.27-5.08 (m, 2H), 4.78 (br dd, J=4.2, 14.6 Hz, 1H), 4.20-4.07 (m, 2H), 4.07-3.95 (m, 1H), 3.81 (d, J=13.8 Hz, 1H), 3.50 (br s, 4H), 2.70-2.52 (m, 4H), 2.44 (q, J=8.8 Hz, 1H), 2.14-2.04 (m, 1H).

### (S)-2-((4-(6-((4-cyano-2-fluorobenzyl)oxy)pyridin-2-yl)piperazin-1-yl)methyl)-1-((1,1-dioxidothietan-2-yl)methyl)-1H-benzo[d]imidazole-6-carboxylic acid (140-P1) and (R)-2-((4-(6-((4-cyano-2-fluorobenzyl)oxy)pyridin-2-yl)piperazin-1-yl)methyl)-1-((1,1-dioxidothietan-2-yl)methyl)-1H-benzo[d]imidazole-6-carboxylic acid (140-P2)

2-((4-(6-((4-cyano-2-fluorobenzyl)oxy)pyridin-2-yl)piperazin-1-yl)methyl)-1-((1,1-dioxidothietan-2-yl)methyl)-1H-benzo[d]imidazole-6-carboxylic acid (140k) was purified by Chiral SFC (column: DAICEL CHIRALPAK AD (250 mm*30 mm, 10 um); mobile phase: [0.1% NH3H2O ETOH]; B %: 45%-45%, min) to give Compound 140-P1 as a white solid. 1H NMR (400 MHz, MeOD-d4) δ 8.35 (s, 1H), 8.00 (dd, J=1.0, 8.6 Hz, 1H), 7.69-7.59 (m, 2H), 7.59-7.52 (m, 2H), 7.48-7.41 (m, 1H), 6.29 (d, J=8.0 Hz, 1H), 6.15 (d, J=7.8 Hz, 1H), 5.44 (s, 2H), 5.25-5.11 (m, 2H), 4.82-4.75 (m, 1H), 4.18-4.09 (m, 2H), 4.06-3.97 (m, 1H), 3.80 (d, J=13.8 Hz, 1H), 3.50 (br t, J=4.8 Hz, 4H), 2.68-2.53 (m, 4H), 2.49-2.39 (m, 1H), 2.15-2.02 (m, 1H).

Compound 140-P2 was obtained as a white solid. 1H NMR (400 MHz, METHANOL-d4) δ 8.38 (s, 1H), 8.00 (d, J=7.8 Hz, 1H), 7.69 (d, J=8.6 Hz, 1H), 7.63 (t, J=7.6 Hz, 1H), 7.60-7.51 (m, 2H), 7.45 (t, J=7.8 Hz, 1H), 6.29 (d, J=8.0 Hz, 1H), 6.15 (d, J=7.8 Hz, 1H), 5.44 (s, 2H), 5.25-5.10 (m, 2H), 4.83-4.74 (m, 1H), 4.19-4.08 (m, 2H), 4.06-3.96 (m, 1H), 3.81 (d, J=13.8 Hz, 1H), 3.50 (br s, 4H), 2.68-2.52 (m, 4H), 2.52-2.40 (m, 1H), 2.14-2.03 (m, 1H).

When a mixture of stereoisomers is separated by HPLC, it is to be appreciated that the resultant individual stereoisomers or mixtures will be arbitrarily assigned. In the examples described herein, when the mixture of stereoisomers is separated by HPLC, it is to be appreciated that an eluting enantiomer or an enantiomer of a resulting compound prepared from the eluting enantiomer is labeled “P1” and another eluting enantiomer or an enantiomer of a resulting compound prepared from the another eluting enantiomer is labeled “P2”. In this example, the eluting enantiomers are of Compound 140. The absolute configuration of the enantiomers, e.g., Compounds 140-P1 & 140-P2 each associated with the corresponding 1H NMR data, may be obtained by known methods.

### Example 141

### 2-(((1R,6S)-6-(6-((2,4-dichlorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid

### and

### 2-(((1S,6R)-6-(6-((2,4-dichlorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid

The title compounds were prepared and can be prepared similarly following the procedures described by General Procedure C.

### 2-(((1R,6S)-6-(6-((2,4-dichlorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (Compound 141-P1)

1H NMR (400 MHz, METHANOL-d4) δ 8.31 (s, 1H), 7.97 (dd, J=1.2, 8.4 Hz, 1H), 7.67 (d, J=8.6 Hz, 1H), 7.57 (t, J=7.8 Hz, 1H), 7.49-7.40 (m, 2H), 7.28 (dd, J=2.0, 8.2 Hz, 1H), 6.91 (d, J=7.4 Hz, 1H), 6.61 (d, J=8.2 Hz, 1H), 5.45-5.35 (m, 2H), 5.27-5.18 (m, 1H), 4.87-4.81 (m, 1H), 4.68 (dd, J=2.4, 15.4 Hz, 1H), 4.63-4.54 (m, 1H), 4.40 (td, J=5.8, 9.0 Hz, 1H), 4.01-3.87 (m, 2H), 2.99 (dd, J=6.2, 11.6 Hz, 1H), 2.81-2.67 (m, 2H), 2.57 (br dd, J=5.8, 13.4 Hz, 1H), 2.52-2.40 (m, 3H), 2.13-2.02 (m, 1H), 1.75 (q, J=6.8 Hz, 1H), 1.18 (dd, J=3.6, 9.0 Hz, 1H), 0.95 (dd, J=3.8, 5.8 Hz, 1H).

### 2-(((1S,6R)-6-(6-((2,4-dichlorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (Compound 141-P2)

1H NMR (400 MHz, METHANOL-d4) δ 8.30 (s, 1H), 7.96 (dd, J=1.2, 8.4 Hz, 1H), 7.65 (d, J=8.4 Hz, 1H), 7.57 (t, J=7.8 Hz, 1H), 7.49-7.40 (m, 2H), 7.28 (dd, J=2.0, 8.2 Hz, 1H), 6.91 (d, J=7.4 Hz, 1H), 6.61 (d, J=8.0 Hz, 1H), 5.40 (d, J=3.8 Hz, 2H), 5.20 (dq, J=2.4, 7.2 Hz, 1H), 4.87-4.81 (m, 1H), 4.69 (dd, J=2.4, 15.2 Hz, 1H), 4.63-4.55 (m, 1H), 4.45 (td, J=6.0, 9.2 Hz, 1H), 4.00 (d, J=13.6 Hz, 1H), 3.83 (d, J=13.8 Hz, 1H), 2.95-2.88 (m, 1H), 2.85-2.79 (m, 1H), 2.78-2.69 (m, 1H), 2.62-2.53 (m, 1H), 2.53-2.46 (m, 1H), 2.43 (t, J=6.0 Hz, 2H), 2.12-2.01 (m, 1H), 1.80-1.72 (m, 1H), 1.18 (dd, J=3.6, 9.2 Hz, 1H), 0.95 (dd, J=3.8, 5.8 Hz, 1H).

When a mixture of stereoisomers is separated by HPLC, it is to be appreciated that the resultant individual stereoisomers or mixtures will be arbitrarily assigned. In the examples described herein, when the mixture of stereoisomers is separated by HPLC, it is to be appreciated that an eluting enantiomer or an enantiomer of a resulting compound prepared from the eluting enantiomer is labeled “P1” and another eluting enantiomer or an enantiomer of a resulting compound prepared from the another eluting enantiomer is labeled “P2”. In this example, the resulting compound is Compound 104. The absolute configuration of the enantiomers, e.g., Compounds 141-P1 & 141-P2 each associated with the corresponding 1H NMR data, may be obtained by known methods.

### Example 142 (General Procedure TT)

### 2-((4-(6-((4-cyano-2-fluorobenzyl)oxy)pyridin-2-yl)piperidin-1-yl)methyl)-1-((1-ethyl-1H-imidazol-5-yl)methyl)-1H-benzo[d]imidazole-6-carboxylic acid

The title compound was prepared according to Scheme 11. This General Procedure TT exemplifies Scheme 11 and provides particular synthetic details as applied to the title compound.

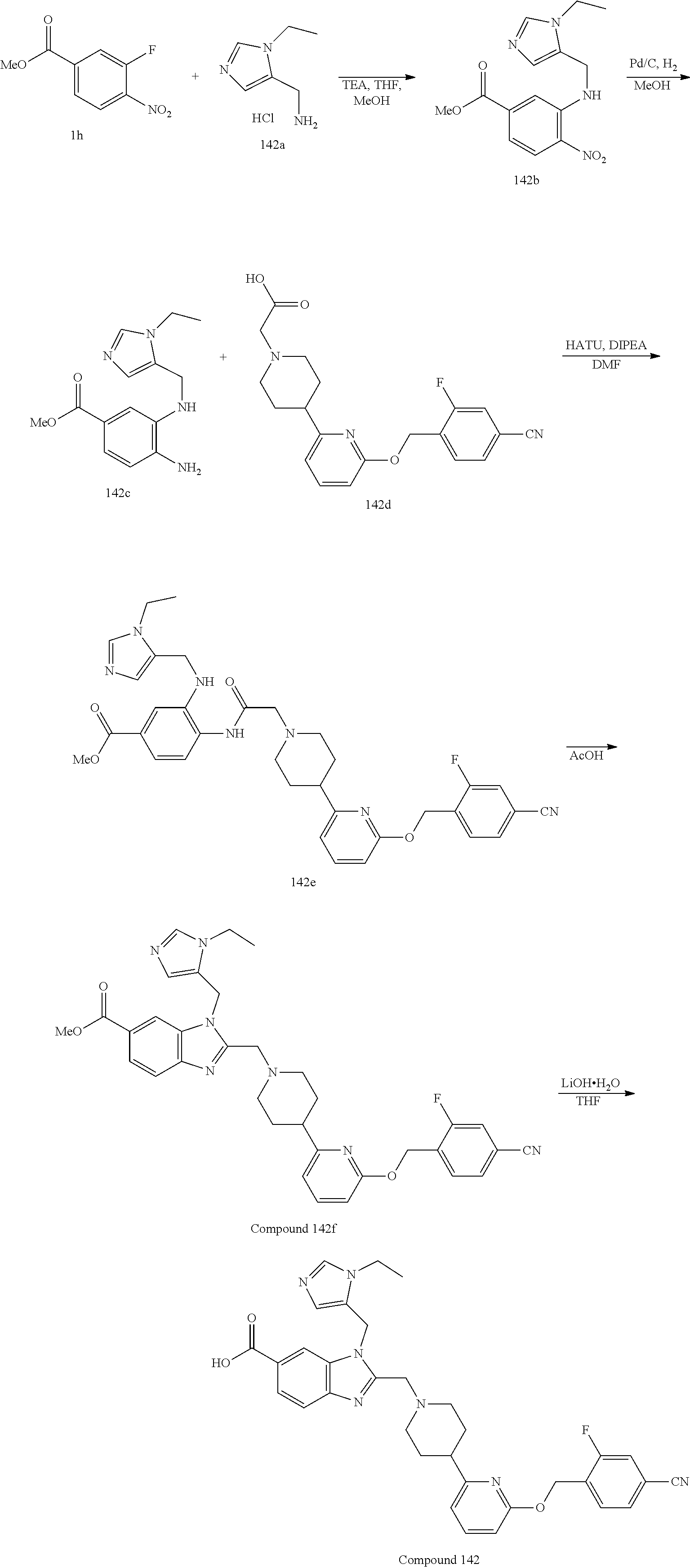

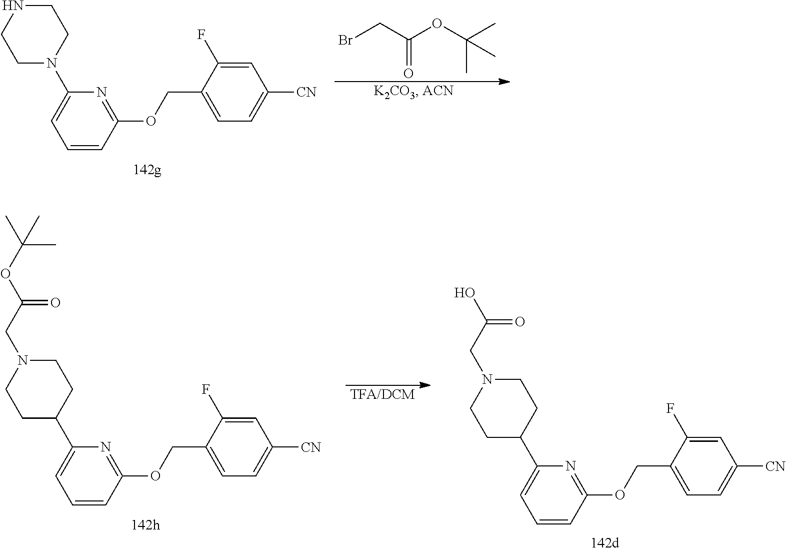

### Methyl 3-(((1-ethyl-1H-imidazol-5-yl)methyl)amino)-4-nitrobenzoate (142b)

To a solution of methyl 3-fluoro-4-nitrobenzoate (1h, 331.74 mg, 1.67 mmol, 1.1 eq) and (1-ethyl-1H-imidazol-5-yl)methanamine (142a, 300 mg, 1.51 mmol, 1 eq, 2HCl) in THF (2.4 mL) and MeOH (1.8 mL) was added TEA (612.98 mg, 6.06 mmol, 843.16 uL, 4eq). The mixture was stirred at 60° C. for 16 hours. LCMS showed 142a was consumed completely and one major peak with desired mass was detected. The reaction mixture was concentrated under reduced pressure to remove solvent. The residue was purified by column chromatography (SiO2, Ethyl acetate:Methanol=10:1 to 5:1) to give 142b as a yellow solid. 1H NMR (400 MHz, CDCl3-d) δ 8.26 (d, J=8.8 Hz, 1H) 7.95 (br s, 1H) 7.70 (d, J=1.6 Hz, 1H) 7.58 (s, 1H) 7.35 (dd, J=8.8, 1.7 Hz, 1H) 7.12 (s, 1H) 4.54 (d, J=5.0 Hz, 2H) 4.01 (q, J=7.4 Hz, 2H) 3.97 (s, 3H) 1.48 (t, J=7.4 Hz, 3H).

### Methyl 4-amino-3-(((1-ethyl-1H-imidazol-5-yl)methyl)amino)benzoate (142c)

To a solution of methyl 3-(((1-ethyl-1H-imidazol-5-yl)methyl)amino)-4-nitrobenzoate (142b, 320 mg, 1.05 mmol, 1 eq) in MeOH (1 mL) was added Pd/C (10% purity, 1.00 eq) and H2 (15 psi). The mixture was stirred at 25° C. for 2 hours. LCMS showed 142b was consumed completely and one major peak with desired mass was detected. The suspension was filtered through a pad of Celite and the pad cake was washed with Ethyl acetate (5 mL*3) to give 142c as a white solid. 1H NMR (400 MHz, MeOH-d4) δ 7.66 (d, J=0.8 Hz, 1H) 7.29-7.39 (m, 2H) 6.97 (s, 1H) 6.67 (d, J=8.0 Hz, 1H) 4.36 (s, 2H) 4.12 (q, J=7.4 Hz, 2H) 3.82 (s, 3H) 1.45 (t, J=7.4 Hz, 3H).

### Methyl 4-(2-(4-(6-((4-cyano-2-fluorobenzyl)oxy)pyridin-2-yl)piperidin-1-yl)acetamido)-3-(((1-ethyl-1H-imidazol-5-yl)methyl)amino)benzoate (142e)

HATU (149.70 mg, 393.70 umol, 1.2 eq) and DIPEA (127.21 mg, 984.26 umol, 171.44 uL, 3 eq) was added to the solution of 2-(4-(6-((4-cyano-2-fluorobenzyl)oxy)pyridin-2-yl)piperidin-1-yl)acetic acid (142d, 142.74 mg, 295.28 umol, 0.9 eq, TFA) in DMF (3 mL) at 25° C. The mixture was stirred at 20° C. for 0.5 hours. Then methyl 4-amino-3-(((1-ethyl-1H-imidazol-5-yl)methyl)amino)benzoate (142c, 90 mg, 328.09 umol, 1 eq) was added to the solution at 20° C. The reaction was stirred at 25° C. for 15.5 hours. TLC (Ethyl acetate:Methanol=1:1) showed 142c was consumed and one new major spot was formed. The residue was purified by column chromatography (SiO2, Ethyl acetate:Methanol=80:1 to 2:1) to give 142e as a yellow solid 1H NMR (400 MHz, MeOH-d4) δ 7.78-7.44 (m, 9H), 6.99 (s, 1H), 6.83 (d, J=7.2 Hz, 1H), 6.70 (d, J=8.2 Hz, 1H), 5.50 (s, 2H), 4.39 (s, 2H), 4.09 (s, 2H), 3.88 (s, 3H), 3.26 (s, 2H), 3.13-3.02 (m, 2H), 2.61 (dt, J=5.6, 10.2 Hz, 1H), 2.38 (dt, J=4.2, 11.0 Hz, 2H), 1.85 (br d, J=5.6 Hz, 4H), 1.40 (br d, J=14.8 Hz, 2H), 1.40 (s, 2H).

### Methyl 2-((4-(6-((4-cyano-2-fluorobenzyl)oxy)pyridin-2-yl)piperidin-1-yl)methyl)-1-((1-ethyl-1H-imidazol-5-yl)methyl)-1H-benzo[d]imidazole-6-carboxylate (142f)

The solution of methyl 4-(2-(4-(6-((4-cyano-2-fluorobenzyl)oxy)pyridin-2-yl)piperidin-1-yl)acetamido)-3-(((1-ethyl-1H-imidazol-5-yl)methyl)amino)benzoate (142e, 130 mg, 207.77 umol, 1 eq) in CH3COOH (3 mL) was stirred at 65° C. for 16 hours. LCMS showed 142e was consumed, and desired mass was detected. The mixture was adjusted to pH=9 with aqueous NaHCO3 (20 mL). The mixture was extracted with Ethyl acetate (10 mL*3). The combined Ethyl acetate was washed with brine (15 mL), dried over Na2SO4, filtered and concentrated to give 142f as a yellow solid. The product was used in next step without further purification. 1H NMR (400 MHz, MeOH-d4) δ 8.15 (d, J=0.9 Hz, 1H), 7.99 (dd, J=1.6, 8.6 Hz, 1H), 7.76-7.71 (m, 2H), 7.70-7.65 (m, 1H), 7.62-7.53 (m, 3H), 6.80 (d, J=7.2 Hz, 1H), 6.68 (d, J=8.2 Hz, 1H), 6.59 (s, 1H), 5.82 (s, 2H), 5.51 (s, 2H), 4.09 (d, J=7.2 Hz, 2H), 3.91 (s, 3H), 3.87 (s, 2H), 2.92 (br d, J=11.4 Hz, 2H), 2.65-2.54 (m, 1H), 2.29-2.19 (m, 2H), 1.83-1.73 (m, 2H), 1.63 (dq, J=3.6, 12.4 Hz, 2H), 1.28 (t, J=7.2 Hz, 4H).

### Tert-butyl 2-(4-(6-((4-cyano-2-fluorobenzyl)oxy)pyridin-2-yl)piperidin-1-yl)acetate (142h)

To a solution of 3-fluoro-4-(((6-(piperidin-4-yl)pyridin-2-yl)oxy)methyl)benzonitrile (142g, 200 mg, 413.60 umol, 1 eq) and tert-butyl 2-bromoacetate (88.74 mg, 454.96 umol, 67.23 uL, 1.1 eq) in ACN (3 mL) was added K2CO3 (285.82 mg, 2.07 mmol, 5 eq). The mixture was stirred at 60° C. for 3 hours. LCMS showed 142g was consumed completely and one main peak with desired mass was detected. The suspension was filtered through a pad of Celite and the pad cake was washed with Ethyl acetate (5 mL*3). The residue was purified by column chromatography (SiO2, Petroleum ether:Ethyl acetate=1:1) to give 142h as white oil.

### 2-(4-(6-((4-cyano-2-fluorobenzyl)oxy)pyridin-2-yl)piperidin-1-yl)acetic acid (142d)

To a solution of tert-butyl 2-(4-(6-((4-cyano-2-fluorobenzyl)oxy)pyridin-2-yl)piperidin-1-yl)acetate (142h, 150 mg, 352.53 umol, 1eq) in DCM (3 mL) and TFA (0.6 mL). The mixture was stirred at 15° C. for 1 hour. LCMS showed 142h was consumed completely and one major peak with desired mass was detected. The reaction mixture was concentrated under reduced pressure to remove solvent to give 142d was obtained as white oil. The crude product was used directly in next step.

### 2-((4-(6-((4-cyano-2-fluorobenzyl)oxy)pyridin-2-yl)piperidin-1-yl)methyl)-1-((1-ethyl-1H-imidazol-5-yl)methyl)-1H-benzo[d]imidazole-6-carboxylic acid (Compound 142)

LiOH·H2O (3.04 mg, 72.41 umol, 1.1 eq) was added to the solution of methyl 2-((4-(6-((4-cyano-2-fluorobenzyl)oxy)pyridin-2-yl)piperidin-1-yl)methyl)-1-((1-ethyl-1H-imidazol-5-yl)methyl)-1H-benzo[d]imidazole-6-carboxylate (142f, 40 mg, 65.82 umol, 1 eq) in THF (2.1 mL) and H2O (0.9 mL) at 20° C. Then the solution was stirred at 20° C. for 16 hours. LCMS showed 142h was consumed completely and one major peak with desired mass was detected. The pH was adjusted to 6-7 with HOAc, and the reaction mixture was concentrated under reduced pressure. The residue was purified by prep-HPLC (column: Waters Xbridge BEH C18 100*25 mm*5 um; mobile phase: [water (10 mM NH4HCO3)-ACN]; B %: 15%-50%, 10 min) to give Compound 142 as white solid. MS mass calculated for [M+H]+ (C33H32FN703) requires m/z 594.3, LCMS found m/z 594.3. 1H NMR (400 MHz, MeOH-d4) δ 8.14 (s, 1H), 7.99 (dd, J=1.2, 8.5 Hz, 1H), 7.79 (s, 1H), 7.74-7.65 (m, 2H), 7.62-7.51 (m, 3H), 6.80 (d, J=7.4 Hz, 1H), 6.70-6.61 (m, 2H), 5.81 (s, 2H), 5.51 (s, 2H), 4.10 (q, J=7.2 Hz, 2H), 3.88 (s, 2H), 2.94 (br d, J=11.4 Hz, 2H), 2.65-2.54 (m, 1H), 2.25 (br t, J=11.0 Hz, 2H), 1.83-1.73 (m, 2H), 1.70-1.57 (m, 2H), 1.28 (t, J=7.4 Hz, 3H).

### Part III: Preparing the Intermediates of Example Compounds

The Intermediates of Example compounds are prepared according to the following procedures.

### Synthesis of Example 2-Intermediate 2c

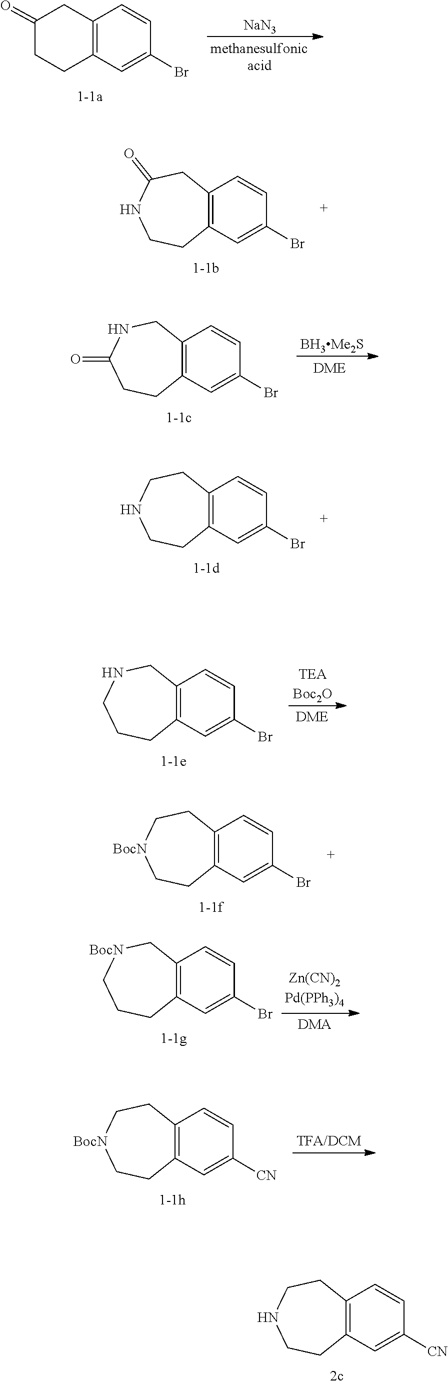

### 7-bromo-4,5-dihydro-1H-benzo[d]azepin-2(3H)-one (1-1b) and 7-bromo-4,5-dihydro-1H-benzo[c]azepin-3(2H)-one (1-1c)

To a solution of 6-bromo-3,4-dihydronaphthalen-2(1H)-one (1-1a, 1g, 4.44 mmol, 1 eq) in methanesulfonic acid (4.7 mL) was slowly added sodium azide (317.71 mg, 4.89 mmol, 1.1 eq) at 0° C. The mixture was stirred at 15° C. for 2 hours. LCMS showed desired mass was detected and 1-1a was consumed completely. The reaction mixture was slowly poured into a solution of potassium hydroxide (4.98 g, 88.8 mmol) in water (80 mL) with vigorous stirring. After the acid was completely quenched, the aqueous solution was extracted with Ethyl acetate (3×500 mL). The organic was dried over Na2SO4, filtered and concentrated under reduced pressure to give a residue. The mixture was used to next step without purification. A mixture of 1-1b and 1-1c were obtained as white solid.

### 7-bromo-2,3,4,5-tetrahydro-1H-benzo[d]azepine (1-1d) and 7-bromo-2,3,4,5-tetrahydro-1H-benzo[c]azepine (1-le)

To a solution of 7-bromo-4,5-dihydro-1H-benzo[d]azepin-2(3H)-one and 7-bromo-4,5-dihydro-1H-benzo[c]azepin-3(2H)-one). (1-1b and 1-1c, 1.06 g, 2.21 mmol, 1 eq) in DME (20 mL) under nitrogen was added a solution of borane; methylsulfanylmethane (10 M, 882.98 uL, 4 eq) at 15° C. and the reaction mixture was stirred for 16 hours at 80° C. LCMS showed desired mass was detected and starting materials were consumed completely. The mixture is quenched with MeOH (100 mL). The reaction mixture was concentrated under reduced pressure to remove solvent and dissolved in hydrogen chloride in methanol solution (HCl 1.25 M in methanol). The mixture is stirred at room temperature for 20 minutes and concentrated under reduced pressure to remove solvent. The residue solid (hydrochloride salt) was used to next step without further purification. A mixture of 1-1d and 1-le were obtained as white solid.

### Tert-butyl 7-bromo-4,5-dihydro-1H-benzo[d]azepine-3(2H)-carboxylate (1-1f) and tert-butyl 7-bromo-4,5-dihydro-1H-benzo[c]azepine-2(3H)-carboxylate (1-1g)

To a solution of 7-bromo-2,3,4,5-tetrahydro-1H-benzo[d]azepine and 7-bromo-2,3,4,5-tetrahydro-1H-benzo[c]azepine (1-1d and 1-le, 1.14 g, 2.17 mmol, 1 eq, HCl) in DCM (20 mL) was added (Boc)20 (1.69 g, 7.74 mmol, 1.78 mL, 3.56 eq) and TEA (2.64 g, 26.05 mmol, 3.63 mL, 12 eq) at 0° C. The mixture was stirred at 15° C. for 2 hours. LCMS showed desired mass was detected and starting materials were consumed completely. The reaction mixture was concentrated under reduced pressure to remove solvent. The residue was diluted with HCl (1 M, 20 mL) and extracted with Ethyl acetate (60 mL). The combined organic layers were washed with brine (10 mL), dried over Na2SO4, filtered and concentrated under reduced pressure to give a residue. The residue was purified by column chromatography (SiO2, Petroleum ether:Ethyl acetate=50:1 to 10:1). 1-1f was obtained as a white solid. 1H NMR (400 MHz, CDCl3-d) δ 7.25 (s, 1H), 6.99 (br d, J=7.6 Hz, 1H), 3.54 (br s, 4H), 2.86 (br s, 4H), 1.49 (s, 9H). 1-1g was obtained as a white solid. 1H NMR (400 MHz, CDCl3-d) δ 7.35-7.28 (m, 2H), 7.26 (d, J=2.1 Hz, 1H), 7.18 (br d, J=8.0 Hz, 1H), 7.08-7.00 (m, 1H), 4.43-4.24 (m, 2H), 3.68 (br d, J=11.2 Hz, 2H), 2.99-2.82 (m, 2H), 1.86-1.69 (m, 2H), 1.40 (s, 9H).

### Tert-butyl 7-cyano-4,5-dihydro-1H-benzo[d]azepine-3(2H)-carboxylate (1-1h)

A mixture of tert-butyl 7-bromo-4,5-dihydro-1H-benzo[d]azepine-3(2H)-carboxylate (1-1f, 100 mg, 306.53 umol, 1 eq), Zn(CN)2 (71.99 mg, 613.07 umol, 38.91 uL, 2 eq), Pd(PPh3)4 (17.71 mg, 15.33 umol, 0.05 eq) in DMA (0.5 mL) was degassed and purged with N2 for 3 times, and then the mixture was stirred at 160° C. for 0.25 hours under N2 atmosphere. TLC (Petroleum ether:Ethyl acetate=5:1) showed the reaction was finished and one new spot was generated. The mixture was added to H2O (10 mL) and extract with MTBE (60 mL). The organic layer was dried over Na2SO4, filtered and concentrated under reduced pressure to give a residue. The residue was purified by prep-TLC (SiO2, Petroleum ether:Ethyl acetate=5:1) to give 1-1h as a white solid. 1H NMR (400 MHz, CDCl3-d) δ 7.50-7.36 (m, 2H), 7.23 (br d, J=7.6 Hz, 1H), 3.57 (br s, 4H), 2.95 (br s, 4H), 1.49 (s, 9H).

### 2,3,4,5-tetrahydro-1H-benzo[d]azepine-7-carbonitrile (2c)

To a solution of tert-butyl 7-cyano-4,5-dihydro-1H-benzo[d]azepine-3(2H)-carboxylate (1-1h, 300 mg, 1.10 mmol, 1 eq) in DCM (10 mL) was added TFA (1.54 g, 13.51 mmol, 1000.00 uL, 12.26 eq) at 15° C., then the mixture was stirred for 2 hours at 15° C. TLC (Petroleum ether:Ethyl acetate=5:1) show one new spot was generated and the 1-1h was consumed completely. The raction was concentrated and added H2O (5 mL), added K2CO3 till pH=9, and extracted with Ethyl acetate (20 mL), dried over Na2SO4, filtered and concentrated under reduced pressure to give a residue. The residue was used to next step without purification. 2c was obtained as a white solid. 1H NMR (400 MHz, CDCl3-d) δ 7.47-7.39 (m, 2H), 7.22 (d, J=7.7 Hz, 1H), 3.04 (s, 8H).

### Synthesis of Example 4-Intermediate 4a

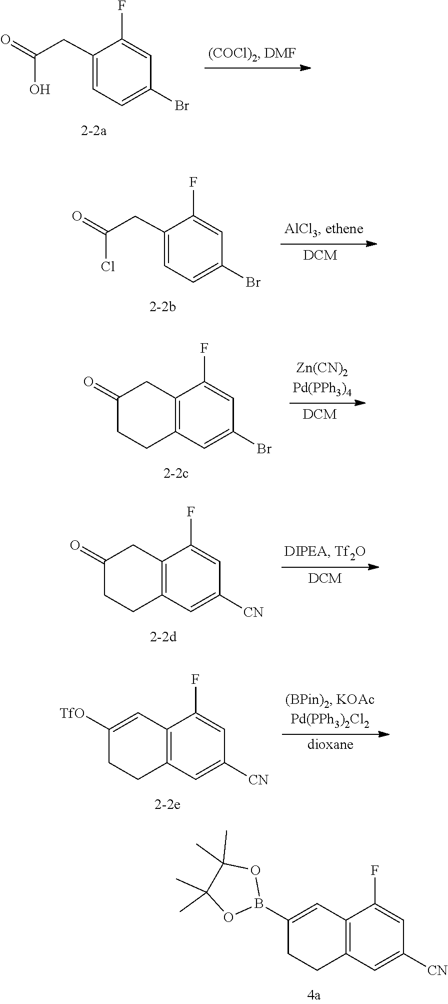 [SMILES: O=C(O)Cc1ccc(Br)cc1F ; O=C(Cl)Cc1ccc(Br)cc1F ; O=C1CCc2cc(Br)cc(F)c2C1 ; N#Cc1cc(F)c2c(c1)CCC(=O)C2 ; N#Cc1cc(F)c2c(c1)CCC(OS(=O)(=O)C(F)(F)F)=C2 ; CC1(C)OB(C2=Cc3c(F)cc(C#N)cc3CC2)OC1(C)C]

### 2-(4-bromo-2-fluorophenyl)acetyl chloride (2-2b)

A solution of 2-(4-bromo-2-fluorophenyl)acetic acid (2-2a, 5 g, 21.46 mmol, 1 eq) and DMF (31.37 mg, 429.12 umol, 33.02 uL, 0.02 eq) in DCM (1 mL) was stirred for 30 min at 15° C., and then oxalyl chloride (3.54 g, 27.89 mmol, 2.44 mL, 1.3 eq) was added slowly at 15° C., the mixture was stirred for 3 hours at 15° C. TLC (Petroleum ether:Ethyl acetate=3:1) showed 2-2a was consumed, and one new spot was generated after quenched with MeOH. The reaction mixture was concentrated under reduced pressure to remove solvent. The residue was used to next step without further purification. 2-2b was obtained as a brown solid.

### 6-bromo-8-fluoro-3,4-dihydronaphthalen-2(1H)-one (2-2c)

To a solution of AlCl3 (3.45 g, 25.85 mmol, 1.41 mL, 1.3 eq) in DCM (10 mL) was added 2-(4-bromo-2-fluorophenyl)acetyl chloride (2-2b, 5 g, 19.88 mmol, 1 eq), and then ethene (557.76 mg, 19.88 mmol, 1 eq) was charged at 13° C. for 3.5 hours. TLC (Petroleum ether:Ethyl acetate=3:1) showed 2-2b was consumed, and one new spot was generated. The reaction mixture was concentrated under reduced pressure to remove solvent. The residue was purified by prep-HPLC (FA condition; column: Phenomenex Luna C18 100*30 mm*5 um; mobile phase: [water (10 mM NH4HCO3)-ACN]; B %: 30%-60%, 9 min) to give 2-2c as a white solid. 1H NMR (400 MHz, CDCl3-d) δ 7.22 (s, 1H), 7.16 (d, J=8.6 Hz, 1H), 3.52 (s, 2H), 3.08 (t, J=6.7 Hz, 2H), 2.71-2.50 (m, 2H).

### 4-fluoro-6-oxo-5,6,7,8-tetrahydronaphthalene-2-carbonitrile (2-2d)

A mixture of 6-bromo-8-fluoro-3,4-dihydronaphthalen-2(1H)-one (2-2c, 100 mg, 411.40 umol, 1 eq), Zn(CN)2 (60.30 mg, 513.52 umol, 32.59 uL, 1.25 eq), Pd(PPh3)4 (23.77 mg, 20.57 umol, 0.05 eq) in DMF (0.5 mL) was degassed and purged with N2 for 3 times, and then the mixture was stirred at 140° C. for 1 hours under N2 atmosphere. TLC (Petroleum ether:Ethyl acetate=3:1) showed 2-2c was consumed, and one new spot was generated. The reaction mixture was added H2O (10 mL) and extracted with MTBE (60 mL). The combined organic layers were dried over Na2SO4, filtered and concentrated under reduced pressure to give a residue. The residue was purified by prep-TLC (SiO2, Petroleum ether:Ethyl acetate=3:1) to give 2-2d as a white solid. 1H NMR (400 MHz, CDCl3-d) δ 7.38 (s, 1H), 7.30 (s, 1H), 3.64 (s, 2H), 3.16 (t, J=6.8 Hz, 2H), 2.71-2.54 (m, 2H).

### 6-cyano-8-fluoro-3,4-dihydronaphthalen-2-yl trifluoromethanesulfonate (2-2e)

A solution of 4-fluoro-6-oxo-5,6,7,8-tetrahydronaphthalene-2-carbonitrile (2-2d, 330 mg, 1.74 mmol, 1 eq) in DCM (10 mL) was added DIPEA (1.13 g, 8.72 mmol, 1.52 mL, 5 eq) at 0° C. dropwised for 10 minutes and then added a solution of trifluoromethylsulfonyl trifluoromethanesulfonate (590.57 mg, 2.09 mmol, 345.36 uL, 1.2 eq) in DCM (10 mL) was added in the mixture dropwised at 0° C., the mixture was stirred for 20 min at 0° C. TLC (Petroleum ether:Ethyl acetate=5:1) showed 2-2d was consumed, and one new spot was formed. The reaction mixture was diluted with DCM (20 mL), and then saturated citric acid (20 mL) was added in the mixture. The mixture was extracted with DCM (60 mL). The organic layer was dried over Na2SO4, filtered and concentrated under reduced pressure to give a residue. The residue was purified by prep-TLC (SiO2, Petroleum ether:Ethyl acetate=5:1) to give 2-2e as a white solid. 1H NMR (400 MHz, CDCl3-d) δ 7.27-7.23 (m, 2H), 6.75 (s, 1H), 3.19-3.05 (m, 2H), 2.83-2.68 (m, 2H).

### 4-fluoro-6-(4,4,5,5-tetramethyl-1,3,2-dioxaborolan-2-yl)-7,8-dihydronaphthalene-2-carbonitrile (4a)

A mixture of 6-cyano-8-fluoro-3,4-dihydronaphthalen-2-yl trifluoromethanesulfonate (2-2e, 600 mg, 1.87 mmol, 1 eq), BPD (616.57 mg, 2.43 mmol, 1.3 eq), KOAc (916.51 mg, 9.34 mmol, 5 eq), Pd (PPh3)2Cl2 (65.55 mg, 93.39 umol, 0.05 eq) in dioxane (20 mL) was degassed and purged with N2 for 3 times, and then the mixture was stirred at 90° C. for 16 hours under N2 atmosphere. TLC (Petroleum ether:Ethyl acetate=5:1) showed 2-2e was consumed, and one new spot was generated. The reaction mixture was concentrated under reduced pressure to remove solvent. The residue was purified by column chromatography (SiO2, Petroleum ether:Ethyl acetate=30:1 to 10:1) to give 4a as a white solid. 1H NMR (400 MHz, CDCl3-d) δ 7.41 (s, 1H), 7.22-7.15 (m, 2H), 2.84-2.72 (m, 2H), 2.44 (dt, J=1.7, 8.2 Hz, 2H), 1.32 (s, 12H), 1.27 (s, 12H).

### Synthesis of Example 12-Intermediate 12c

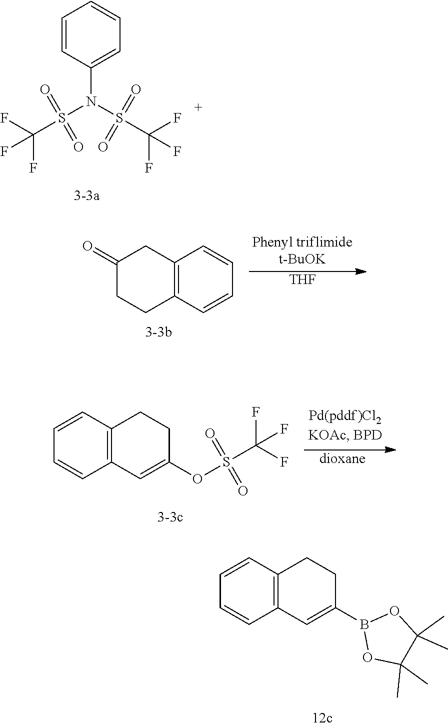

### 3-3a 3-3b 3-3c 12c 3,4-dihydronaphthalen-2-yl trifluoromethanesulfonate (3-3c)

A solution of 3,4-dihydronaphthalen-2(1H)-one (3-3b, 2.15 g, 14.70 mmol, 1.95 mL, 1.05 eq) in THF (120 mL) was cooled to −20° C. in a cooling bath (i-PrOH/dry-ice). Then t-BuOK (1 M, 14.70 mL, 1.05 eq) was added in the solution slowly over 10 minutes. After completing the addition, the mixture was warmed to 0° C. in an ice-water bath and stirred for 1 hour. Afterward, the mixture was cooled to −20° C. in a cooling bath (i-PrOH/dry-ice). Then 1,1,1-trifluoro-N-phenyl-N-((trifluoromethyl)sulfonyl)methanesulfonamide (3-3a, 5 g, 14.00 mmol, 1 eq) was added in the mixture over one minutes. The mixture was warmed to 0° C. in an ice-water bath and stirred for 4 hours. TLC (Petroleum ether:Ethyl acetate=20:1) indicated 3-3b was consumed, and one new spot was generated. The mixture is concentrated under reduced pressure to approximately one-fourth of the original volume in a rotary evaporator. The aqueous phase was extracted with ethyl acetate (30 mL*3). The combined organic phase was washed with brine (25 mL), dried with anhydrous Na2SO4, filtered and concentrated in vacuo. The crude product was purified by column chromatography (SiO2, Petroleum ether:Ethyl acetate=99:1 to 50:1) to give 3-3c as a white solid. 1H NMR (400 MHz, CDCl3-d) δ 7.23-7.19 (m, 2H), 7.17-7.14 (m, 1H), 7.10-7.07 (m, 1H), 6.49 (s, 1H), 3.07 (t, J=8.4 Hz, 3H), 2.75-2.67 (m, 2H).

### 2-(3,4-dihydronaphthalen-2-yl)-4,4,5,5-tetramethyl-1,3,2-dioxaborolane (3-3d)

A mixture of 3,4-dihydronaphthalen-2-yl trifluoromethanesulfonate (3-3c, 500 mg, 1.80 mmol, 1 eq), BPD (684.47 mg, 2.70 mmol, 1.5 eq), Pd(dppf)Cl₂ (105.19 mg, 143.76 umol, 0.08 eq) and KOAc (529.07 mg, 5.39 mmol, 3 eq) in dioxane (4 mL) was degassed and purged with N2 for 3 times, and then the mixture was stirred at 80° C. for 10 hours under N2 atmosphere. TLC (Petroleum ether:Ethyl acetate=20:1) indicated 3-3d was consumed, and one new spot was generated. The aqueous phase was extracted with ethyl acetate (30 mL*3). The combined organic phase was washed with brine (30 mL), dried with anhydrous Na2SO4, filtered and concentrated in vacuo. The crude product was purified by column chromatography on silica gel eluted with Petroleum ether:Ethyl acetate=99:1 to 20:1) to give 3-3d as a white solid. 1H NMR (400 MHz, CDCl3-d) δ 7.22-7.08 (m, 5H), 2.76 (t, J=8.0 Hz, 2H), 2.40 (dt, J=1.4, 8.0 Hz, 2H), 1.33 (s, 12H).

### Synthesis of Example 66-Intermediate

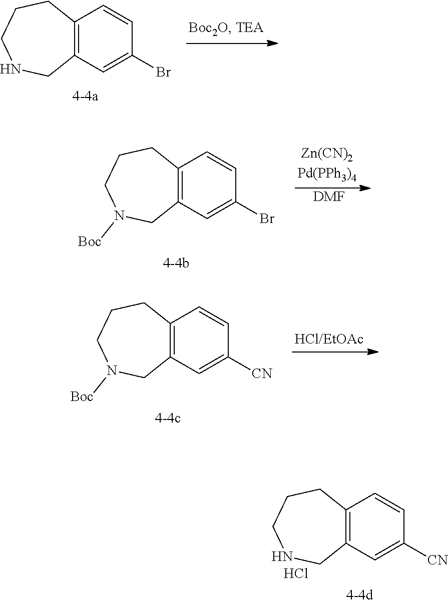

### Tert-butyl 8-bromo-4,5-dihydro-1H-benzo[c]azepine-2(3H)-carboxylate (4-4b)

Boc2O (965.21 mg, 4.42 mmol, 1.02 mL, 2 eq) was added to the solution of 8-bromo-2,3,4,5-tetrahydro-1H-benzo[c]azepine (4-4a, 0.5 g, 2.21 mmol, 1 eq) and TEA (671.27 mg, 6.63 mmol, 923.35 uL, 3 eq) in DCM (20 mL) at 0° C. Then the solution was stirred at 20° C. for 1 hours. TLC (Petroleum ether:Ethyl acetate=5:1) showed 4-4a was consumed, and one new spot was generated. The mixture was adjusted to pH=7 with HCl (1 M). The mixture was extracted with DCM (10 mL*3). The combined DCM was washed with brine (15 mL), dried over Na2SO4, filtered and concentrated. The residue was purified by column chromatography (SiO2, Petroleum ether:Ethyl acetate=80:1 to 20:1) to give 4-4b as a white solid. 1H NMR (400 MHz, MeOD-d4) δ 7.43-7.34 (m, 1H), 7.33-7.26 (m, 1H), 7.08 (br d, J=7.8 Hz, 1H), 4.37 (s, 2H), 3.69 (br s, 2H), 3.02-2.91 (m, 2H), 1.72 (br d, J=5.0 Hz, 2H), 1.38 (s, 9H).

### Tert-butyl 8-cyano-4,5-dihydro-1H-benzo[c]azepine-2(3H)-carboxylate (4-4c)

Zn(CN)2 (390.18 mg, 3.32 mmol, 210.91 uL, 2 eq) was added to the solution of tert-butyl 8-bromo-4,5-dihydro-1H-benzo[c]azepine-2(3H)-carboxylate (4-4b, 542 mg, 1.66 mmol, 1 eq) and Pd(PPh3)4 (191.99 mg, 166.14 umol, 0.1 eq) in DMF (10 mL) at 20° C. The solution was stirred at 90° C. for 3.5 hours under N2. TLC (Petroleum ether:Ethyl acetate=5:1) showed 4-4b was consumed, and one new spot was generated. The mixture was extracted with ethyl acetate (20 mL*3). The combined ethyl acetate was washed with H2O (25 mL *2), brine (15 mL), dried over Na2SO4, filtered and concentrated. The residue was purified by column chromatography (SiO2, Petroleum ether:Ethyl acetate=80:1 to 20:1). 4-4c was obtained as white solid. 1H NMR (400 MHz, MeOD-d4) δ 7.62-7.50 (m, 2H), 7.41-7.30 (m, 1H), 4.46 (s, 2H), 3.72 (br s, 2H), 3.13-3.03 (m, 2H), 1.80-1.67 (m, 2H), 1.38 (s, 9H).

### 2,3,4,5-tetrahydro-1H-benzo[c]azepine-8-carbonitrile hydrochloride (4-4d)

The solution of tert-butyl 8-cyano-4,5-dihydro-1H-benzo[c]azepine-2(3H)-carboxylate (4-4c, 230 mg, 844.53 umol, 1 eq) in HCl/Ethyl acetate (8 mL) was stirred at 20° C. for 0.5 hours. LCMS detected the desired mass and showed 4-4c was consumed. The mixture was concentrated to remove the solvent to give 4-4d as a white solid. The product was used to next step without purification. 1H NMR (400 MHz, MeOD-d4) δ 7.79 (d, J=1.5 Hz, 1H), 7.72 (dd, J=1.8, 7.8 Hz, 1H), 7.50 (d, J=7.8 Hz, 1H), 4.46 (s, 2H), 3.55-3.49 (m, 2H), 3.20-3.13 (m, 2H), 2.06-1.97 (m, 2H).

### Synthesis of Example 71-Intermediate

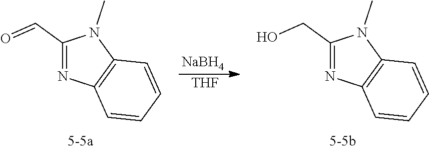

### (1-methyl-1H-benzo[d]imidazol-2-yl)methanol (5-5b)

To a solution of 1-methyl-1H-benzo[d]imidazole-2-carbaldehyde (5-5a, 150 mg, 936.49 umol, 1 eq) in THF (3 mL) was added NaBH4 (38.97 mg, 1.03 mmol, 1.1 eq) at 20° C. under N2. The mixture was stirred at 20° C. for 1 hour. LCMS showed 5-5a was consumed completely and desired mass was detected. The reaction mixture was quenched by addition water (30 mL) at 20° C. The aqueous phase was extracted with ethyl acetate (25 mL*2). The combined organic phase was washed with brine (20 mL), dried with anhydrous Na2SO4, filtered and concentrated in vacuo. The residue was purified by prep-TLC (ethyl acetate:methanol=20:1) to give 5-5b as a white solid. 1H NMR (400 MHz, CDCl3-d) δ 7.77-7.67 (m, 1H), 7.34-7.28 (m, 2H), 7.27-7.23 (m, 1H), 4.91 (s, 2H), 3.83 (s, 3H).

### Synthesis of Example 72-Intermediate

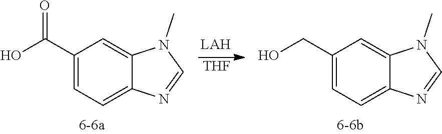

### (1-methyl-1H-benzo[d]imidazol-6-yl)methanol (6-6b)

To a solution of 1-methyl-1H-benzo[d]imidazole-6-carboxylic acid (6-6a, 300 mg, 1.70 mmol, 1 eq) in THF (6 mL) was added LAH (161.58 mg, 4.26 mmol, 2.5 eq). The mixture was stirred at 20° C. for 16 hours. LCMS showed 6-6a was remained and desired compound was detected. The reaction was cooled to room temperature and quenched by addition water (0.1 mL), followed by 0.1 mL 15 percent NaOH, and followed by 0.2 mL water. The mixture was stirred vigorously for 1 hour. The organic layers were filtered and concentrated under reduced pressure to give a residue. The residue was purified by prep-TLC (SiO2, ethyl acetate:methanol=5:1) to give 6-6b as a yellow oil. 1H NMR (400 MHz, CDCl3-d) δ 7.93 (s, 1H), 7.76 (d, J=8.3 Hz, 1H), 7.46 (s, 1H), 7.27-7.32 (m, 1H), 4.86 (s, 2H), 3.84 (s, 3H).

### Synthesis of Example 73-Intermediate

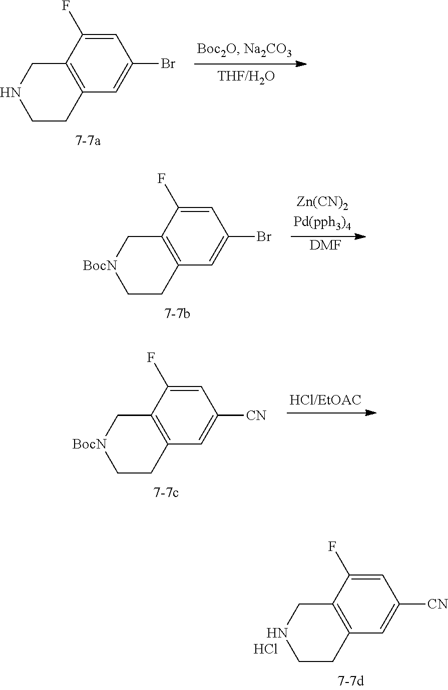

### Tert-butyl 6-bromo-8-fluoro-3,4-dihydroisoquinoline-2(1H)-carboxylate (7-7b)

To a solution of 6-bromo-8-fluoro-1,2,3,4-tetrahydroisoquinoline (7-7a, 320 mg, 1.39 mmol, 1 eq) in THF (5 mL) and H2O (5 mL) was added Na2CO3 (294.83 mg, 2.78 mmol, 2 eq) and tert-butoxycarbonyl tert-butyl carbonate (607.09 mg, 2.78 mmol, 639.04 uL, 2 eq). The mixture was stirred at 20° C. for 16 hours. TLC (petroleum ether:ethyl acetate=2:1) showed 7-7a was consumed completely. The aqueous phase was extracted with ethyl acetate (15 mL*3). The combined organic phase was washed with brine (20 mL), dried with anhydrous Na2SO4, filtered and concentrated in vacuo. The crude product was purified by column chromatography (SiO2, petroleum ether:ethyl acetate=15:1 to 1:1) to give 7-7b as light yellow oil. 1H NMR (400 MHz, CDCl3-d) δ 7.14-7.03 (m, 2H), 4.52 (br s, 2H), 3.64 (br t, J=5.4 Hz, 2H), 2.82 (br t, J=5.0 Hz, 2H), 1.50 (s, 9H).

### Tert-butyl 6-cyano-8-fluoro-3,4-dihydroisoquinoline-2(1H)-carboxylate (7-7c)

A mixture of tert-butyl 6-bromo-8-fluoro-3,4-dihydroisoquinoline-2(1H)-carboxylate (7-7b, 290 mg, 878.28 umol, 1 eq), Zn(CN)2 (206.26 mg, 1.76 mmol, 111.49 uL, 2 eq), Pd(PPh3)4 (101.49 mg, 87.83 umol, 0.1 eq) in DMF (8 mL) was degassed and purged with N2 for 3 times, and then the mixture was stirred at 120° C. for 16 hours under N2 atmosphere. LCMS showed 7-7b was consumed completely and desired mass was detected. The mixture was filtered and the filtrate was washed with water (15 mL*2) and brine (15 mL*2). The organic layer was dried by Na2SO4, filtrated and concentrated in vacuo. The filter cake was quenched by NaClO3 (aq) (20 mL). The crude product was purified by column chromatography (SiO2, petroleum ether:ethyl acetate=15:1 to 1:1) to give 7-7c as a white solid. 1H NMR (400 MHz, MeOD-d4) δ 7.44-7.35 (m, 2H), 4.63 (s, 2H), 3.67 (t, J=5.8 Hz, 2H), 2.90 (t, J=5.8 Hz, 2H), 1.50 (s, 9H).

### 8-fluoro-1,2,3,4-tetrahydroisoquinoline-6-carbonitrile (7-7d)

A mixture of tert-butyl 6-cyano-8-fluoro-3,4-dihydroisoquinoline-2(1H)-carboxylate (7-7c, 110 mg, 398.11 umol, 1 eq) in HCl/Ethyl acetate (4 M, 1 mL) was stirred at 20° C. for 1 hour. TLC (petroleum ether:ethyl acetate=2:1) showed 7-7c was consumed completely. The mixture was concentrated in vacuo to give 7-7d as a white solid. 1H NMR (400 MHz, DMSO-d6) δ 7.78 (d, J=9.9 Hz, 1H), 7.68 (s, 1H), 4.36 (s, 2H), 3.40-3.35 (m, 2H), 3.06 (t, J=6.0 Hz, 2H).

### Synthesis of Example 74-Intermediate

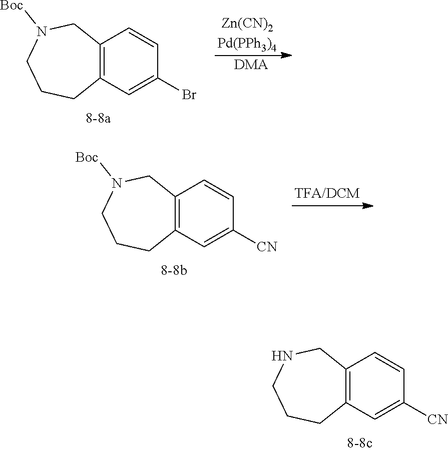

### Tert-butyl 7-cyano-4,5-dihydro-1H-benzo[c]azepine-2(3H)-carboxylate (8-8b)

A mixture of tert-butyl 7-bromo-4,5-dihydro-1H-benzo[c]azepine-2(3H)-carboxylate (8-8a, 100 mg, 306.53 umol, 1 eq), Zn(CN)2 (72 mg, 613.07 umol, 38.91 uL, 2 eq), Pd(PPh3)4 (17.71 mg, 15.33 umol, 0.05 eq) in DMA (2 mL) was degassed and purged with N2 for 3 times, and then the mixture was stirred at 160° C. for 0.25 hours under N2 atmosphere. TLC (Petroleum ether:Ethyl acetate=5:1) showed the reaction was finished and one new spot was generated. The mixture was added H2O (10 mL) and extract with MTBE (60 mL). The organic layer was dried over Na2SO4, filtered and concentrated under reduced pressure. The residue was purified by column chromatography (SiO2, Petroleum ether:Ethyl acetate=20:1 to 10:1) to give 8-8b as a white solid. 1H NMR (400 MHz, CDCl3-d) δ 7.50-7.38 (m, 2H), 7.28 (br s, 1H), 7.27-7.23 (m, 1H), 4.54-4.32 (m, 2H), 3.71 (br d, J=6.8 Hz, 2H), 2.98 (br d, J=5.8 Hz, 2H), 1.80 (br d, J=5.2 Hz, 2H), 1.39 (s, 9H).

### 2,3,4,5-tetrahydro-1H-benzo[c]azepine-7-carbonitrile (8-8c)

To a solution of tert-butyl 7-cyano-4,5-dihydro-1H-benzo[c]azepine-2(3H)-carboxylate (8-8b, 250 mg, 917.97 umol, 1 eq) in DCM (10 mL) was added TFA (1.43 g, 12.51 mmol, 925.93 uL, 13.6 eq). The mixture was stirred at 15° C. for 2 hours. TLC (Petroleum ether:Ethyl acetate=5:1) show one new spot was generated and the 8-8b was consumed completely. The reaction mixture was concentrated under reduced pressure to give 8-8c as a white solid. 1H NMR (400 MHz, CDCl3-d) δ 7.53-7.46 (m, 2H), 7.29 (d, J=8.4 Hz, 1H), 4.14 (s, 2H), 3.39-3.29 (m, 2H), 3.08-2.96 (m, 2H), 1.92 (br t, J=5.0 Hz, 2H).

### Synthesis of Example 75-Intermediate

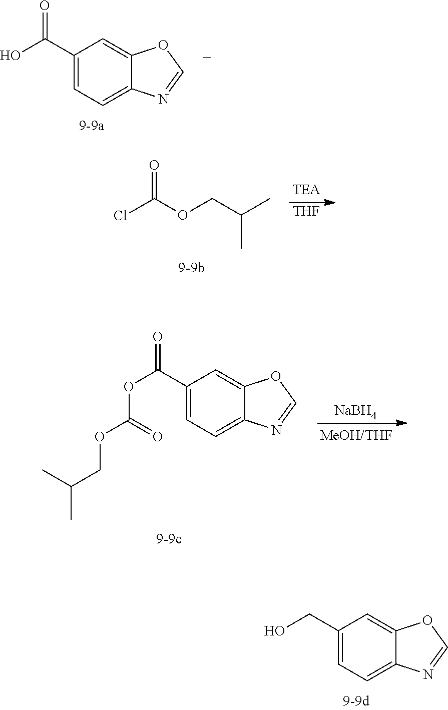

### benzo[d]oxazole-6-carboxylic (isobutyl carbonic) anhydride (9-9c)

To a stirred solution of benzo[d]oxazole-6-carboxylic acid (9-9a, 250 mg, 1.53 mmol, 1 eq) in THF (5 mL) was added TEA (465.23 mg, 4.60 mmol, 639.93 uL, 3 eq) and isobutyl carbonochloridate (9-9b, 313.96 mg, 2.30 mmol, 301.88 uL, 1.5 eq) at 0° C. under N2 atmosphere. The mixture was stirred at 20° C. for 1 hour. One drop of reaction mixture was quenched with MeOH, and TLC (Petroleum ether:Ethyl acetate=0:1) indicated 9-9a was consumed completely and one new spot was formed. The mixture was used into the next step without further purification. The mixture of 9-9c in THF was obtained as white oil.

### Benzo[d]oxazol-6-ylmethanol (9-9d)

To a solution of isobutoxycarbonyl benzo[d]oxazole-6-carboxylic (isobutyl carbonic) anhydride (9-9c, 400 mg, 1.52 mmol, 1 eq) in THF (5 mL) was added NaBH4 (114.96 mg, 3.04 mmol, 2 eq) and MeOH (5 mL) at 0° C. The mixture was stirred at 20° C. for 0.5 hours. LCMS showed 9-9c was consumed completely and desired mass was detected. The reaction mixture was quenched by addition H2O (5 mL). The reaction mixture was concentrated under reduced pressure to remove THF and MeOH. The residue water layer was extracted with Ethyl acetate (10 mL*3). The combined organic layers were washed with brine (5 mL*2), dried over Na2SO4, filtered and concentrated under reduced pressure. The residue was purified by prep-TLC (SiO2, Petroleum ether:Ethyl acetate=1:1) to give 9-9d as a light yellow solid. 1H NMR (400 MHz, DMSO-d6) δ 8.69 (s, 1H), 7.77-7.65 (m, 2H), 7.35 (d, J=8.4 Hz, 1H), 5.35 (t, J=5.8 Hz, 1H), 4.63 (d, J=5.8 Hz, 2H).

### Synthesis of Example 78 and 79-Intermediate

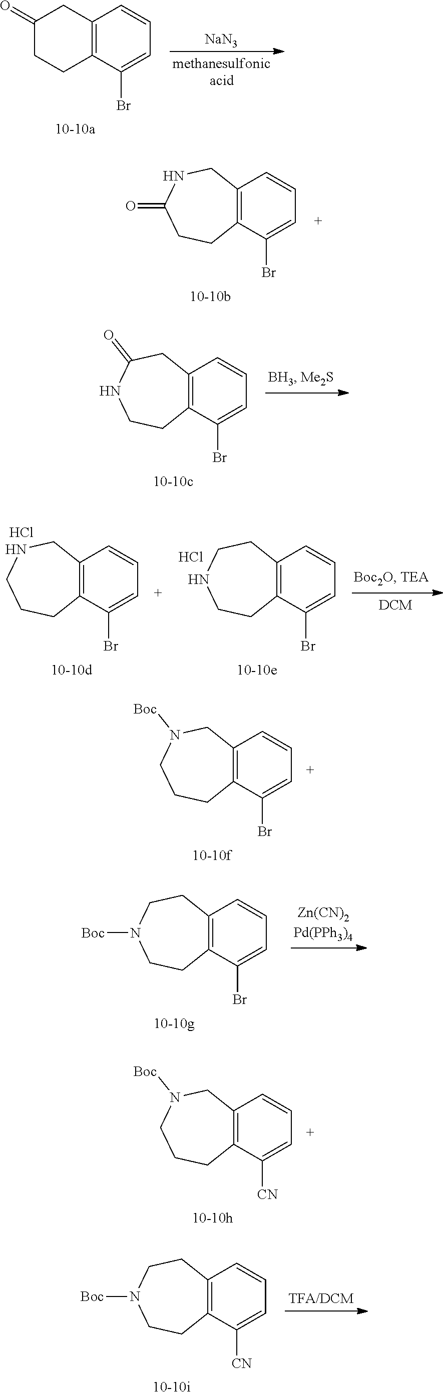

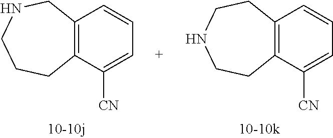 [SMILES: N#Cc1cccc2c1CCCNC2 ; N#Cc1cccc2c1CCNCC2]

### 6-bromo-4,5-dihydro-1H-benzo[c]azepin-3(2H)-one (10-10b) & 6-bromo-4,5-dihydro-1H-benzo[d]azepin-2(3H)-one (10-10c)

To a solution of 5-bromo-3,4-dihydronaphthalen-2(1H)-one (10-10a, 1g, 4.44 mmol, 1 eq) in methanesulfonic acid (8 mL) was added sodium azide (317.71 mg, 4.89 mmol, 1.1 eq) slowly at 0° C. The mixture was stirred at 0-10° C. for 2 hours. LCMS showed desired mass was detected and 10-10a was consumed completely. To the mixture was added NaOH (aq. 4 M) until pH=9-10. Then the mixture was filtered and concentrated under reduced pressure to give a mixture of 10-10c and 10-10b as white solid.

### 6-bromo-2,3,4,5-tetrahydro-1H-benzo[c]azepine (10-10d) & 6-bromo-2,3,4,5-tetrahydro-1H-benzo[d]azepine (10-10e)

To a solution of 6-bromo-4,5-dihydro-1H-benzo[c]azepin-3(2H)-one (10-10b) & 6-bromo-4,5-dihydro-1H-benzo[d]azepin-2(3H)-one (10-10b, 800 mg, 3.33 mmol, 1 eq) in DME (20 mL) was added BH3-Me2S (10 M, 666.40 uL, 2 eq) at 0° C. The mixture was stirred at 80° C. for 16 hours. LCMS showed starting materials were consumed, and desired mass was detected. The mixture was quenched with MeOH (100 mL). The reaction mixture was concentrated under reduced pressure, and the residue was dissolved in hydrogen chloride in methanol solution (HCl 1.25 M in methanol). The mixture was stirred at room temperature for 20 minutes and concentrated under reduced pressure to remove solvent to give a mixture of Compound 10-10d & 10-10e as a white solid.

### Tert-butyl 6-bromo-4,5-dihydro-1H-benzo[c]azepine-2(3H)-carboxylate (10-10f) & tert-butyl 6-bromo-4,5-dihydro-1H-benzo[d]azepine-3(2H)-carboxylate (10-10g)

To a solution of 6-bromo-2,3,4,5-tetrahydro-1H-benzo[c]azepine (10-10d) & 6-bromo-2,3,4,5-tetrahydro-1H-benzo[d]azepine (10-10e, 800 mg, 3.05 mmol, 1 eq, HCl) in DCM (8 mL) was added TEA (1.54 g, 15.23 mmol, 2.12 mL, 5 eq) and (Boc)20 (731.44 mg, 3.35 mmol, 769.94 uL, 1.1 eq). The mixture was stirred at 15° C. for 1 hour. LCMS showed desired mass was detected and 10-10d &10-10e were consumed completely. DCM (20 mL) and citric acid solution (10%, 10 mL) were added to the reaction mixture. The mixture was extracted with DCM (100 mL). The organic layers were washed with brine (10 mL), dried over Na2SO4 filtered and concentrated under reduced pressure to give a residue. The residue was purified by column chromatography (SiO2, Petroleum ether:Ethyl acetate=20:1 to 10:1) to give a mixture of 10-10f & 10-10g as colourless oil. 1H NMR (400 MHz, CDCl3-d) δ 7.52-7.39 (m, 1H), 7.17-7.03 (m, 1H), 7.03-6.92 (m, 1H), 4.53-4.33 (m, 1H), 3.79-3.50 (m, 3H), 3.20 (br d, J=4.8 Hz, 2H), 2.94 (br d, J=5.2 Hz, 1H), 1.77 (br d, J=4.8 Hz, 1H), 1.50-1.37 (m, 9H).

### Tert-butyl 6-cyano-4,5-dihydro-1H-benzo[c]azepine-2(3H)-carboxylate (10-10h) & tert-butyl 6-cyano-4,5-dihydro-1H-benzo[d]azepine-3(2H)-carboxylate (10-10i)

To a solution of tert-butyl 6-bromo-4,5-dihydro-1H-benzo[c]azepine-2(3H)-carboxylate (10-10f) & tert-butyl 6-bromo-4,5-dihydro-1H-benzo[d]azepine-3(2H)-carboxylate (10-10g, 211 mg, 646.79 umol, 1 eq) in DMA (1.5 mL) was added Zn(CN)2 (151.90 mg, 1.29 mmol, 82.11 uL, 2 eq) and Pd(PPh3)4 (37.37 mg, 32.34 umol, 0.05 eq). The mixture was stirred at 160° C. for 0.25 hours. TLC (Petroleum ether:Ethyl acetate=10:1) showed one new spot was generated and 10-10f & 10-10 g were consumed completely. The reaction mixture was added H2O (10 mL) and extracted with MTBE (60 mL), the organic layer was dried over Na2SO4, filtered and concentrated under reduced pressure to give a mixture of 10-10h & 10-10i as a white solid. 1H NMR (400 MHz, CDCl3-d) δ 7.52 (br t, J=8.4 Hz, 1H), 7.44-7.32 (m, 1H), 7.26-7.20 (m, 1H), 4.53-4.33 (m, 1H), 3.73 (br d, J=7.8 Hz, 1H), 3.61 (br d, J=5.0 Hz, 2H), 3.33-3.15 (m, 2H), 2.96 (br s, 1H), 1.84 (br d, J=5.0 Hz, 1H), 1.48 (s, 5H), 1.39 (s, 4H).

### 2,3,4,5-tetrahydro-1H-benzo[c]azepine-6-carbonitrile (10-10j) &2,3,4,5-tetrahydro-1H-benzo[d]azepine-6-carbonitrile (10-10k)

To a solution of tert-butyl 6-cyano-4,5-dihydro-1H-benzo[c]azepine-2(3H)-carboxylate (10-10h) & tert-butyl 6-cyano-4,5-dihydro-1H-benzo[d]azepine-3(2H)-carboxylate (10-10i, 170 mg, 624.22 umol, 1 eq) in DCM (3 mL) was added TFA (462.00 mg, 4.05 mmol, 0.3 mL, 6.49 eq). The mixture was stirred at 15° C. for 2 hours. TLC (Petroleum ether:Ethyl acetate=5:1) showed one new spot was generated and 10-10h & 10-10i were consumed completely. The reaction was concentrated and diluted with H2O (5 mL). Then K2CO3 was added in the mixture until pH=9, and the mixture was extracted with Ethyl acetate (20 mL), dried over Na2SO4, filtered and concentrated under reduced pressure to give a mixture of 10-10j & 10-10k as a yellow solid. 1H NMR (400 MHz, CDCl3-d) δ 7.55-7.44 (m, 1H), 7.39-7.30 (m, 1H), 7.25-7.15 (m, 1H), 3.99 (s, 1H), 3.37-3.19 (m, 3H), 3.06-3.00 (m, 1H), 2.99 (s, 1H), 1.91 (br s, 2H).

### Synthesis of Example 80-Intermediate

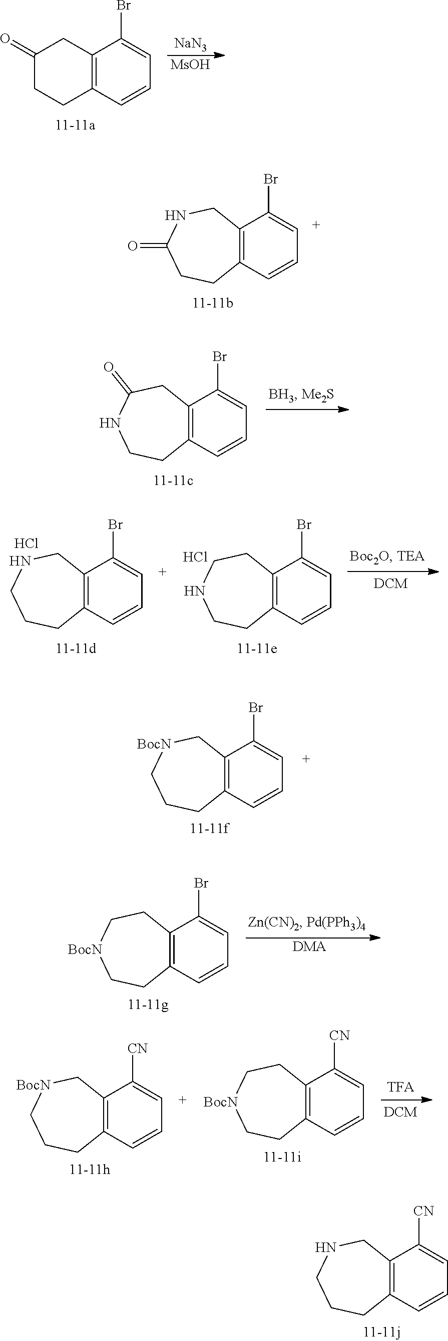

### 9-bromo-4,5-dihydro-1H-benzo[c]azepin-3(2H)-one (11-11b) and 9-bromo-4,5-dihydro-1H-benzo[d]azepin-2(3H)-one (11-11c)

To a solution of 8-bromo-3,4-dihydronaphthalen-2(1H)-one (11-11a, 1g, 4.44 mmol, 1 eq) in MsOH (5 mL) was slowly added NaN3 (346.59 mg, 5.33 mmol, 1.2 eq) at 0° C. The mixture was stirred at 0° C. for 2 hours. LCMS showed 11-11a was consumed completely and desired mass was detected. NaOH aqueous solution (4 M, 20 mL) was added to the mixture dropwise, and white solids were formed. The solids were collected by filtration, and washed with water (10 mL*3). The mixture of 11-11b and 11-11c was obtained as a white solid.

### 9-bromo-2,3,4,5-tetrahydro-1H-benzo[c]azepine (11-11d) and 6-bromo-2,3,4,5-tetrahydro-1H-benzo[d]azepine (11-11e)

To a solution of the mixture of 9-bromo-4,5-dihydro-1H-benzo[c]azepin-3(2H)-one and 9-bromo-4,5-dihydro-1H-benzo[d]azepin-2(3H)-one (11-11b and 11-11b, 1.2 g, 5.00 mmol, 1 eq) in THF (25 mL) was added BH3-Me2S (10 M, 499.80 uL, 1 eq) at 0° C. The mixture was stirred at 80° C. for 16 hours. LCMS showed one main peak with desired mass was detected. The reaction mixture was quenched by addition MeOH (100 mL), and then stirred at 20° C. for 0.5 hours. The mixture was concentrated under reduced pressure to remove solvent. The residue was dissolved by HCl/MeOH (5 mL), then concentrated under reduced pressure to remove solvent to give the mixture of 11-11d and 11-11e as a white solid, the products was used directly for the next step without purification.

### Tert-butyl 9-bromo-4,5-dihydro-1H-benzo[c]azepine-2(3H)-carboxylate (11-11f) and tert-butyl 6-bromo-4,5-dihydro-1H-benzo[d]azepine-3(2H)-carboxylate (11-11g)

To a solution of the mixture of 9-bromo-2,3,4,5-tetrahydro-1H-benzo[c]azepine and 6-bromo-2,3,4,5-tetrahydro-1H-benzo[d]azepine (11-11d and 11-11e, 1.13 g, 5.00 mmol, 1 eq) in DCM (20 mL) was added TEA (1.52 g, 14.99 mmol, 2.09 mL, 3 eq) and Boc2O (1.20 g, 5.50 mmol, 1.26 mL, 1.1 eq) dropwise. The mixture was stirred at 20° C. for 2 hours. TLC (Petroleum ether:Ethyl acetate=5:1) indicated 11-d & 11-e were consumed completely and one new spot was formed. The reaction mixture was extracted with DCM (30 mL*3) and H2O (30 mL). The combined organic layers were washed with brine (20 mL), dried over anhydrous Na2SO4, filtered and concentrated under reduced pressure. The residue was purified by column chromatography (SiO2, Petroleum ether:Ethyl acetate=10:1) to give the mixture of 11-11f and 11-1g as colorless oil. 1H NMR (400 MHz, CDCl3-d) δ 7.36-7.49 (m, 1H), 7.03-7.11 (m, 1H), 6.98 (td, J=7.6, 5.8 Hz, 1H), 4.72 (br s, 1H), 3.68 (br s, 1H), 3.52-3.63 (m, 2H), 3.12-3.25 (m, 1H), 2.84-3.02 (m, 2H), 1.88 (br s, 1H), 1.38-1.50 (m, 9 H).

### Tert-butyl 9-cyano-4,5-dihydro-1H-benzo[c]azepine-2(3H)-carboxylate (11-11h) and tert-butyl 6-cyano-4,5-dihydro-1H-benzo[d]azepine-3(2H)-carboxylate (11-11i)

To a solution of the mixture of tert-butyl 9-bromo-4,5-dihydro-1H-benzo[c]azepine-2(3H)-carboxylate and tert-butyl 6-bromo-4,5-dihydro-1H-benzo[d]azepine-3(2H)-carboxylate (11-11f and 11-11g, 200 mg, 613.07 umol, 1 eq) in DMA (1 mL) was added Zn(CN)2 (143.98 mg, 1.23 mmol, 77.83 uL, 2 eq) and Pd(PPh3)4 (35.42 mg, 30.65 umol, 0.05 eq). The mixture was stirred at 160° C. for 15 min under N2. TLC (Petroleum ether:Ethyl acetate=5:1, 12) showed 11-11f was consumed completely and one major peak with desired mass was detected. The reaction mixture was extracted with Ethyl acetate (10 mL*3) and H2O (10 mL). The combined organic layers were washed with brine (5 mL), dried over anhydrous Na2SO4, filtered and concentrated under reduced pressure. The aqueous phase was quenched by NaClO (5 mL). The residue was purified by column chromatography (SiO2, Petroleum ether:Ethyl acetate=5:1) to give the mixture of 11-1 h and 11-11i as colourless oil. Then the mixture was purified by prep-HPLC (column: Phenomenex Luna C18 200*40 mm*10 um; mobile phase: [water (10 mM NH4HCO3)-ACN]; B %: 50%-70%, 8 min) to give 11-11h as colorless gum. 1H NMR (400 MHz, CDCl3-d) δ 7.49 (dd, J=7.8, 1.0 Hz, 1H), 7.39 (br d, J=7.4 Hz, 1H), 7.23-7.26 (m, 1H), 4.72 (s, 2H), 3.74 (br s, 2H), 2.94-3.02 (m, 2H), 1.77-1.90 (m, 2H), 1.43 (s, 9H).

### 2,3,4,5-tetrahydro-1H-benzo[c]azepine-9-carbonitrile (11-1 j)

The solution of tert-butyl 9-cyano-4,5-dihydro-1H-benzo[c]azepine-2(3H)-carboxylate (11-11h, 60 mg, 220.31 umol, 1 eq) in TFA (0.1 mL) and DCM (1 mL) was stirred at 20° C. for 2 hours. TLC (Petroleum ether:Ethyl acetate=5:1) indicated 11-1 h was consumed completely and one new spot formed. The reaction mixture was concentrated under reduced pressure to remove solvent to give 11-11j as white solid. 1H NMR (400 MHz, DMSO-d6) δ 7.79 (d, J=7.6 Hz, 1H), 7.64 (d, J=7.4 Hz, 1H), 7.53 (t, J=7.8 Hz, 1H), 4.50 (br s, 2H), 4.32 (br s, 1H), 3.44 (br s, 1H), 2.98-3.17 (m, 2H), 1.85 (br s, 2H).

### Synthesis of Example 83 and 84-Intermediate

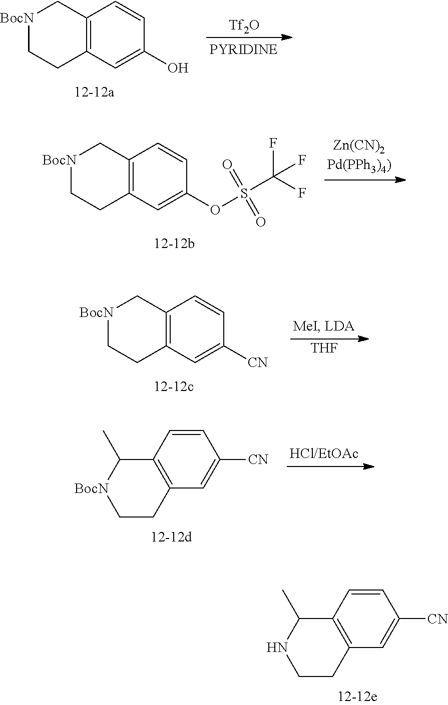

### Tert-butyl 6-(((trifluoromethyl)sulfonyl)oxy)-3,4-dihydroisoquinoline-2(1H)-carboxylate (12-12b)

To a solution of tert-butyl 6-hydroxy-3,4-dihydroisoquinoline-2(1H)-carboxylate (12-12a, 700 mg, 2.81 mmol, 1 eq) in pyridin (2 mL) was added Tf20 (871.41 mg, 3.09 mmol, 509.60 uL, 1.1 eq) at 0° C. The mixture was stirred at 0° C. for 0.5 hours. TLC (Petroleum ether:Ethyl acetate=3:1) showed starting material was consumed completely. The mixture was concentrated under reduced pressure to remove pyridine. The residue was purified by column chromatography (SiO2, Petroleum ether:Ethyl acetate=4:1 to 1:1) to give 12-12b as a yellow solid. 1H NMR (400 MHz, CDCl3-d) δ 7.23-7.15 (m, 1H), 7.14-7.05 (m, 2H), 4.59 (s, 2H), 3.66 (br t, J=5.6 Hz, 2H), 2.87 (br t, J=5.8 Hz, 2H), 1.50 (s, 9H).

### Tert-butyl 6-cyano-3,4-dihydroisoquinoline-2(1H)-carboxylate (12-12c)

To a solution of tert-butyl 6-(((trifluoromethyl)sulfonyl)oxy)-3,4-dihydroisoquinoline-2(1H)-carboxylate (12-12b, 900 mg, 2.36 mmol, 1 eq) in DMF (5 mL) was added Zn(CN)2 (554.25 mg, 4.72 mmol, 299.60 uL, 2 eq) and Pd(PPh3)4 (272.70 mg, 235.99 umol, 0.1 eq) at 20° C. under N2. The mixture was stirred at 80° C. for 16 hours. TLC (Petroleum ether:Ethyl acetate=3:1) showed 12-12b was consumed completely. The mixture was filtered and the filtrate was washed with water (40 mL*2). The mixture was extracted with MTBE (40 mL *2). The combined organic layers were washed with brine (30 mL*3), dried with anhydrous Na2SO4, filtered and concentrated under reduced pressure to give a residue. The filter cake was quenched by NaClO(aq)(50 ml). The residue was purified by column chromatography (SiO2, Petroleum ether:Ethyl acetate=5:1 to 2:1) to give 12-12 was obtaind as a white solid. 1H NMR (400 MHz, CDCl3-d) δ 7.45-7.45 (m, 1H), 7.50-7.43 (m, 1H), 7.21 (d, J=7.8 Hz, 1H), 4.62 (s, 2H), 3.67 (br t, J=5.8 Hz, 2H), 2.87 (br t, J=5.7 Hz, 2H), 1.50 (s, 9H).

### Tert-butyl 6-cyano-1-methyl-3,4-dihydroisoquinoline-2(1H)-carboxylate (12-12d)

To a solution of tert-butyl 6-cyano-3,4-dihydroisoquinoline-2(1H)-carboxylate (12-12c, 150 mg, 580.69 umol, 1 eq) in THF (5 mL) was added MeI (82.42 mg, 580.69 umol, 36.15 uL, 1 eq) and LDA (2 M, 319.38 uL, 1.1 eq) at −65° C. under N2. The mixture was stirred at −65° C. for 1 hr. TLC (Petroleum ether:Ethyl acetate=3:1) showed 12-12c was consumed completely. The mixture was quenched by NH4Cl (25 mL). The aqueous phase was extracted with ethyl acetate (30 mL*3). The combined organic phase was washed with brine (20 mL), dried with anhydrous Na2SO4, filtered and concentrated in vacuo. The residue was purified by prep-TLC (Petroleum ether:Ethyl acetate=3:1) to give 12-12d as a white solid. 1H NMR (400 MHz, CDCl3-d) δ 7.50-7.40 (m, 2H), 7.22 (d, J=8.1 Hz, 1H), 5.49-5.01 (m, 1H), 4.40-3.95 (m, 1H), 3.38-3.04 (m, 1H), 3.03-2.83 (m, 1H), 2.82-2.67 (m, 1H), 1.50 (s, 9H), 1.46 (d, J=6.8 Hz, 3H).

### 1-methyl-1,2,3,4-tetrahydroisoquinoline-6-carbonitrile (12-12e)

To a solution of tert-butyl 6-cyano-1-methyl-3,4-dihydroisoquinoline-2(1H)-carboxylate (12-12d, 135 mg, 495.70 umol, 1 eq) in HCl/Ethyl acetate (4 mL) at 25° C. under N2. The mixture was stirred at 25° C. for 1 hour. LCMS showed reactant was consumed completely and desired mass was detected. The mixture was concentrated in vacuo to give 12-12e as a white solid. 1H NMR (400 MHz, CDCl3-d) δ 7.52-7.52 (m, 1H), 7.61-7.48 (m, 1H), 7.31-7.31 (m, 1H), 7.30 (br d, J=8.4 Hz, 1H), 4.66 (ddd, J=3.0, 4.4, 6.0 Hz, 1H), 3.65-3.51 (m, 1H), 3.48-3.37 (m, 1H), 3.34-3.17 (m, 2H), 1.89 (br d, J=6.6 Hz, 3H).

### Part III: Biological Examples

Biological data reported in Table 2 for the compounds whose stereochemistry is noted as arbitrarily assigned in the Example compounds can be associated with the appropriate Example compound by reference to the corresponding 1H NMR data. It is thus possible that the compound associated with a given 1H NMR and biological data set will have the same absolute stereochemistry or a different absolute stereochemistry from the compound whose stereochemistry is noted as arbitrarily assigned in the Example compounds. Biological data is also reported in Table 3.

### Biological Assays: GLP-1R Cell Assays

### GLP-1R Cell Assay 1

GLP-1R mediated agonist activity was determined using the cAMP Hunter CHO-K1 GLP1R Gs cell line (DiscoverX, cat #95-0062C2), a stable CHO-K1 derived cell lines overexpressing naturally Gs coupled wild-type human GLP-1R (accession number: NM_002062.3). GLP-1R agonism in this cell line was detected by measuring cellular cAMP levels using the HitHunter cAMP assay kit (cat #90-0075SM2), a homogenous, gain-of-signal competitive immune assay based on Enzyme Fragment Complementation (EFC) technology. In this system cellular cAMP competes with exogenous labeled cAMP (ED-cAMP) for binding to an anti-cAMP antibody. Under conditions of high cellular cAMP (GLP-1R agonism), ED-cAMP is free to complement inactive enzyme reagent (EA) to form an active enzymatic complex that produces a luminescent signal upon addition of substrate reagents. The luminescent signal is directly proportional to the amount of cellular cAMP.

For compound testing, cells were seeded in a total volume of 20 μL in white walled, 384-well microplates and incubated at 37° C. prior to compound testing. Prior to compound addition, cell culture media was removed by aspiration and replaced with 15 μL reagent buffer (2:1 HBSS/10 mM HEPES: cAMP XS+Ab reagent). Compounds were dissolved in DSMO and serially diluted (3-fold) prior to final dilution to 4× in assay buffer. Samples (5 μL) were added to cells and incubated at 37° C. for 30-60 min prior to 1 hour incubation with 20 μL cAMP XS+ED/CL lysis reagent and 3 hour incubation with 20 μL cAMP XS+EA reagent at room temperature. Microplates were read on a PerkinElmer Envision instrument. Compound activity was analyzed using CBIS data analysis suite (ChemInnovation). Percent activity was calculated based on duplicate assay runs and normalized to the positive control Exendin-4, a GLP-1R peptide agonist, using the following formula:

$$\% Activity = 100 \% ⋆ \frac{( mean RLU of test sample - mean RLU of vehicle control )}{( mean RLU of Max control - mean RLU of vehicle control )}$$

The effective compound concentration resulting in 50% response (EC50) was determined by non-linear regression analysis of dose-response curves.

### GLP-1R Cell Assay 2

Stable cell lines expressing high and low GLP-1R surface expression were generated in CHO-K1 cells transfected (Fugene 6) with a puromycin selectable DNA plasmid encoding human GLP-1R receptor (accession number: NM_002062.5) under control of an EF1A promoter. Transfected cells were seeded into 24-well plates (9,000 cells/well) containing complete medium and incubated in a humidified incubator at 37° C. with 5% carbon dioxide. After overnight incubation, medium was replaced with complete medium supplemented with puromycin (6 μg/mL) and refreshed every 2-3 days to select for stably transfected cells. Individual pools of selected cells were expanded prior to analysis for responsiveness to GLP-1 control peptide using a TR-FRET assay to detect cAMP (LANCE Ultra cAMP Assay, Perkin Elmer). Briefly, cells were collected in Versene solution, plated in 384-well plates (1,000 cells/well) and combined with serially diluted GLP-1R control peptide (10 nL) using an acoustic dispenser (ECHO). Plates were incubated for 30 minutes at 25° C. prior to the addition of EU-cAMP tracer (5 μL) and Ulight-anti-cAMP (5 μL) reagents to each well, followed by 15 minutes incubation at 25° C. TR-FRET signal was detected using an EnVision Multimode Plate Reader (excitation=320 nm; emission=615 and 655 nm). Dose-response curves were used to generate EC₅₀ values as a measure of responsiveness to the GLP-1R control peptide. Selected cell lines were monitored for responsiveness over multiple passages to ensure stability. CHO-K1_hGLP-1Rhigh_clone16 and CHO-K1_hGLP-1Rlow_clone10 showed consistently high and low responsiveness to GLP-1R control peptide, respectively, and were chosen for further analysis to determine relative levels of GLP-1R surface expression. Briefly, GLP-1R expression was analyzed by flow cytometry using a fluorescein-labeled Exendin-4 peptide fluorescent probe (FLEX). Cells were harvested in Versene solution and washed 3-times with PBS+0.5% BSA before incubation with FLEX reagent (10 μM) for 2 hours at room temperature. After incubation, cells were washed 3-times in PBS+0.5% BSA before final resuspension in PBS prior to analysis by flow cytometry to measure FLEX mean fluorescence intensity (MFI) as a measure of GLP-1R expression on the cell surface. Both cell lines showed higher MFI values relative to control CHO-K1 cells, confirming GLP-1R surface expression; CHO-K1_hGLP-1Rhigh_clone16 cells showed significantly higher MFI levels relative to CHO-K1-hGLP-1low_clone10 cells.

For compound testing in the CHO-K1_hGLP-1Rlow_clone10 cell lines, cells were seeded in 384-well plates (1,000 cells/well). Test compounds were serially diluted in DMSO (10-point, 3-fold dilution), added to wells using an ECHO dispenser (10 nL/well) and plates were centrifuged for 1 min and agitated for 2 min at room temperature prior to 30-minute incubation at 25° C. After incubation, Eu-cAMP (5 μL) and Ulight-anti-cAMP (5 μL) reagents were added to each well, followed by centrifugation for 1 minute, agitation for 2 minutes at room temperature, and final incubation of the plates at 25° C. for 15 minutes. Plates were read using an EnVision microplate reader (excitation=320 nm; emission=615 and 655 nm). Dose-response curves were generated from duplicate wells based on percent activation calculated relative to a control GLP-1 peptide agonist that was run in parallel. EC₅₀ values were determined by fitting percent activation as a function of compound concentration using the Hill equation (XLfit).

The EC50 values of exemplary compounds are shown in Table 2 below. The compounds tested were compound samples prepared according to procedures described in the Synthetic Examples section, with the stereochemical purity as indicated in the Examples.

TABLE 2

| | GLP-1R Cell Assay 1 |
|---|---|
| Example | EC₅₀ (nM) |
| 1 | 42.4766 |
| 2 | >500 |
| 3 | 9.0482 |
| 4 | 4.5811 |
| 5 | 314.5440 |
| 6-P1 | 247.7990 |
| 6-P2 | >500 |
| 7-P1 | 5.1930 |
| 7-P2 | >500 |
| 8-P1 | >500 |
| 8-P2 | 82.1334 |
| 9 | 0.4763 |
| 9-P1 | 0.1711 |
| 9-P2 | 0.4944 |
| 10 | 13.8570 |
| 11 | 0.2570 |
| 12 | 124.2030 |
| 13 | 0.7883 |
| 14 | 0.2464 |
| 15 | 0.3110 |
| 16 | 9.2446 |
| 17 | 23.0351 |
| 18 | >500 |
| 19 | 41.2107 |
| 20-P1 | >500 |
| 20-P2 | 165.9770 |
| 21 | 2771.5300 |
| 22 | 3739.7100 |
| 23 | >500 |
| 24 | 349.8670 |
| 25 | >500 |
| 26-P1 | >500 |
| 26-P2 | >500 |
| 27 | 232.1730 |
| 28 | >500 |
| 29 | 16.0620 |
| 30 | 16.2765 |
| 31 | >500 |
| 32 | >500 |
| 33 | >500 |
| 34 | 333.2580 |
| 35 | >500 |
| 36 | 36.4418 |
| 37 | >500 |
| 38 | >500 |
| 39 | 420.1650 |
| 40 | 3.2834 |
| 41 | >500 |
| 42 | 3.8409 |
| 43 | >500 |
| 44 | 82.1240 |
| 45 | 21.6840 |
| 46 | >500 |
| 47 | >500 |
| 48 | >500 |
| 49 | >500 |
| 50 | 201.9930 |
| 51 | 348.4290 |
| 52 | >500 |
| 53 | 7.6961 |
| 54 | >500 |
| 55 | 42.3136 |
| 56 | >500 |
| 57 | >500 |
| 58 | 0.2209 |
| 59 | 227.8400 |
| 60 | 8.1796 |
| 61 | >500 |
| 62 | 0.8596 |
| 63 | 54.4615 |
| 64 | >500 |
| 65 | 0.9980 |
| 66 | 134.9190 |
| 67 | 0.8490 |
| 68 | 2.0308 |
| 69 | 0.3942 |
| 70 | 1.1933 |
| 71 | 15.7311 |
| 72 | 4.5952 |
| 73 | 1.4256 |
| 74 | 141.1320 |
| 75 | 2.1698 |
| 76 | >500 |
| 77 | 116.8190 |
| 78 | 56.1565 |
| 79 | 206.3010 |
| 80 | 37.9167 |
| 81 | 207.2120 |
| 82 | 2.7770 |
| 83 | 228.5850 |
| 84 | 101.9590 |
| 85 | 6.1125 |
| 86 | 75.3321 |
| 87 | >500 |
| 88 | 238.9240 |
| 89 | 76.2200 |
| 90 | >500 |
| 91 | 17.3403 |
| 92 | 8.3505 |
| 93 | >500 |
| 94 | 152.2000 |
| 95 | 46.6359 |
| 96 | 78.1358 |
| 97 | 18.6243 |
| 98 | 0.5565 |
| 99 | 2070.8200 |
| 100 | 245.9230 |
| 101 | 28.7376 |
| 102 | 3.9378 |
| 103 | 5.5395 |
| 104-P1 | 4.9077 |
| 104-P2 | 8.1220 |
| 105 | >500 |
| 106 | >500 |
| 107 | 1.0667 |
| 108 | 4.1550 |
| 109 | 3.6529 |
| 110 | 0.2127 |
| 111 | 36.8282 |
| 112 | 1.2433 |
| 113 | 0.1759 |
| 114 | 5.1062 |
| 115 | 8.0039 |
| 116 | 4.2736 |
| 117 | 5.9332 |
| 118 | 185.1150 |
| 119 | 0.5520 |
| 120 | 43.5472 |
| 121-P1 | 0.0329 |
| 121-P2 | 0.0386 |
| 122 | 2.2360 |
| 123 | 1.0683 |
| 124 | 22.8934 |
| 125 | 0.0329 |
| 126-P1 | 0.1735 |
| 126-P2 | 0.3733 |
| 127 | 1.0945 |
| 128 | 0.2460 |
| 129 | 2.5989 |
| 130 | 0.9663 |
| 131 | 1.7113 |
| 132 | 33.9907 |
| 133 | 10.6135 |
| 134 | 0.2221 |
| 135 | 4.8781 |
| 136 | 108.5688 |
| 137 | 0.0514 |
| 138 | 0.1076 |
| 139 | 2.6544 |
| 140-P1 | 0.5710 |
| 140-P2 | 0.1697 |
| 142 | 0.008333 |

The EC50 values of other exemplary compounds are shown in Table 3 below. The compounds tested were compound samples prepared according to procedures analogous to those described in the Synthetic Examples section. Stereochemical information was arbitrarily assigned; molecules for which no stereochemical designation is shown were generated as a mixture of stereoisomers and were tested as a mixture of stereoisomers.

TABLE 3

| | GLP-1R Cell Assay 1 | GLP-1R Cell Assay 2 |
|---|---|---|
| Cmpd No. | EC50 (nM) | EC50 (nM) |
| 143 | >500 | |
| 144 | >500 | |
| 145 | >500 | |
| 146-P1 | >500 | |
| 146-P2 | 7.0213 | |
| 147 | >500 | |
| 148 | >500 | |
| 149 | >500 | |
| 150 | 0.9005 | |
| 151-P1 | 260.5866 | |
| 151-P2 | >500 | |
| 152 | >500 | |
| 153 | >500 | |
| 154 | >500 | |
| 155 | >500 | |
| 156 | 87.10 | |
| 157 | >500 | |
| 158 | >500 | |
| 159 | >500 | |
| 160 | >500 | |
| 161 | 304.0332 | |
| 162 | >500 | |
| 163-P1 | 0.2660 | |
| 163-P2 | 0.4143 | |
| 164 | 0.0254 | |
| 165 | 0.0254 | |
| 166 | 59.3678 | |
| 167 | 265.6519 | |
| 168 | 17.4381 | |
| 169 | | 1.1367 |
| 170 | | 120 |
| 171 | | 1408.3473 |
| 172 | | 119.2906 |
| 173 | | 13.1687 |
| 174 | | 63.4305 |
| 175 | | 14.5175 |
| 176 | | 1590.2583 |
| 177 | | 6.7255 |
| 178 | | 7.5565 |
| 179 | | 36.6042 |
| 180 | | 214.5258 |
| 181 | | 22.7706 |
| 182 | | 33.9147 |
| 183 | | >10000 |
| 184-P1 | 0.2661 | |
| 184-P2 | 0.4143 | |
| 185-P1 | | 1.6908 |
| 185-P2 | | 18.0631 |
| 186-P1 | | 645.8092 |
| 186-P2 | | >10000 |
| 187 | | 13.1687 |

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

1. A compound of Formula (V): 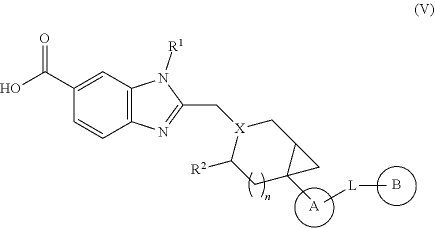
or a pharmaceutically acceptable salt thereof, wherein:
X is N or CH;
n is 0 or 1;
R¹ is —C₁-C₆ alkylene-R⁵, wherein R⁵ is 3- to 6-membered heterocyclyl or 5- to 6-membered heteroaryl, each of which is independently optionally substituted by C₁-C₆ alkyl;
R² is hydrogen, oxo, or C₁-C₆ alkyl;
Ring A is 5- to 12-membered heterocyclyl or 5- to 12-membered heteroaryl, each of which is independently optionally substituted by halo, CN, C₃-C₆ cycloalkyl, or C₁-C₆ alkyl optionally substituted by halo or OH;
L is a bond, —O—, C₁-C₆ alkylene, *—O—C₁-C₆ alkylene-**, *—C₁-C₆ alkylene-O—**, or *—NR⁶—C₁-C₆ alkylene-**, wherein
* represents the point of attachment to ring A and ** represents the point of attachment to ring B,
when L is *—O—C₁-C₆ alkylene-**, the C₁-C₆ alkylene is optionally substituted by R^{L}, wherein:
each R^{L} is independently C₁-C₆ alkyl or halo, or
two R^{L} are taken together with the carbon atom or atoms to which they are attached to form C₃-C₆ cycloalkyl or 3- to 6-membered heterocyclyl;
when L is C₁-C₆ alkylene, the C₁-C₆ alkylene is optionally substituted by R^{L1} wherein:
each R^{L1} is independently halo, OH, or C₁-C₆ alkyl; or
two R^{L} are taken together with the carbon atom or atoms to which they are attached to form C₃-C₆ cycloalkyl or 3- to 6-membered heterocyclyl, and
R⁶ is hydrogen or C₁-C₆ alkyl; and
Ring B is C₃-C₁₀ cycloalkyl, C₆-C₁₄ aryl, 4- to 12-membered heterocyclyl, or 5- to 12-membered heteroaryl, each of which is independently optionally substituted by one to three substituents independently selected from the group consisting of halo, CN, oxo, C₁-C₆ alkyl, C₁-C₆ haloalkyl, —COCH₃, —CONH₂, —S(O)₂CH3, and phenyl.

2. The compound of claim 1, wherein the compound is of formula (Va) or (Vb) 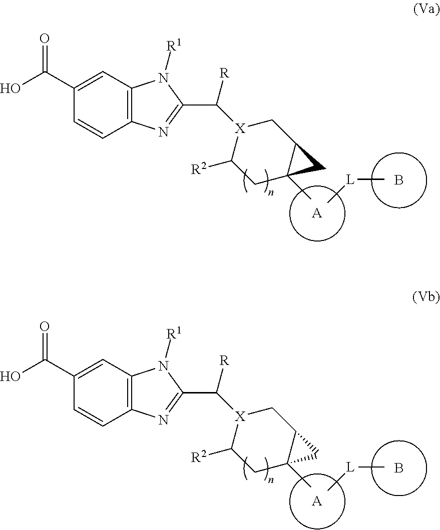
or a pharmaceutically acceptable salt thereof.

3. The compound of claim 1 or 2, or a pharmaceutically acceptable salt thereof, wherein R¹ is —CH₂—R⁵.

4. The compound of any one of claims 1-3, or a pharmaceutically acceptable salt thereof, wherein R⁵ is 3- to 6-membered heterocyclyl, which is optionally substituted by C₁-C₆ alkyl.

5. The compound of any one of claims 1-4, or a pharmaceutically acceptable salt thereof, wherein R⁵ is 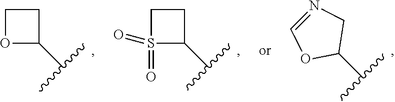 [SMILES: *C1CCO1 ; *C1CCS1(=O)=O ; *C1CN=CO1]
each of which is independently optionally substituted by C₁-C₆ alkyl.

6. The compound of any one of claims 1-5, or a pharmaceutically acceptable salt thereof, wherein R⁵ is

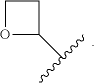 [SMILES: *C1CCO1]

7. The compound of any one of claims 1-3, or a pharmaceutically acceptable salt thereof, wherein R⁵ is 5- to 6-membered heteroaryl, which is optionally substituted by C₁-C₆ alkyl.

8. The compound of any one of claims 1-3 or 7, or a pharmaceutically acceptable salt thereof, wherein R⁵ is 5-membered heteroaryl, which is optionally substituted by C₁-C₆ alkyl.

9. The compound of any one of claims 1-3 or 7-8, or a pharmaceutically acceptable salt thereof, wherein R⁵ is 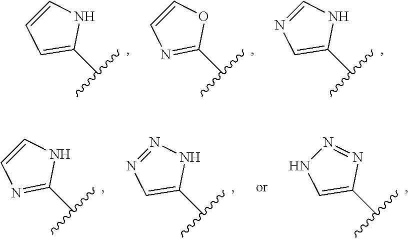 [SMILES: *c1ccc[nH]1 ; *c1ncco1 ; *c1cnc[nH]1 ; *c1ncc[nH]1 ; *c1cnn[nH]1 ; *c1c[nH]nn1]
each of which is optionally substituted by C₁-C₆ alkyl.

10. The compound of any one of claims 1-3 or 7-9, or a pharmaceutically acceptable salt thereof, wherein R⁵ is 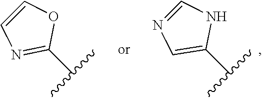 [SMILES: *c1ncco1 ; *c1cnc[nH]1]
each of which is optionally substituted by C₁-C₆ alkyl.

11. The compound of any one of claims 1-10, or a pharmaceutically acceptable salt thereof, wherein X is N.

12. The compound of any one of claims 1-11, or a pharmaceutically acceptable salt thereof, wherein n is 1.

13. The compound of any of one of claims 1-12, or a pharmaceutically acceptable salt thereof, wherein Ring A is 5- to 12-membered heterocyclyl, which is optionally substituted by halo, CN, C₃-C₆ cycloalkyl, or C₁-C₆ alkyl optionally substituted by halo or OH.

14. The compound of any one of claims 1-13, or a pharmaceutically acceptable salt thereof, wherein Ring A is Ring A is 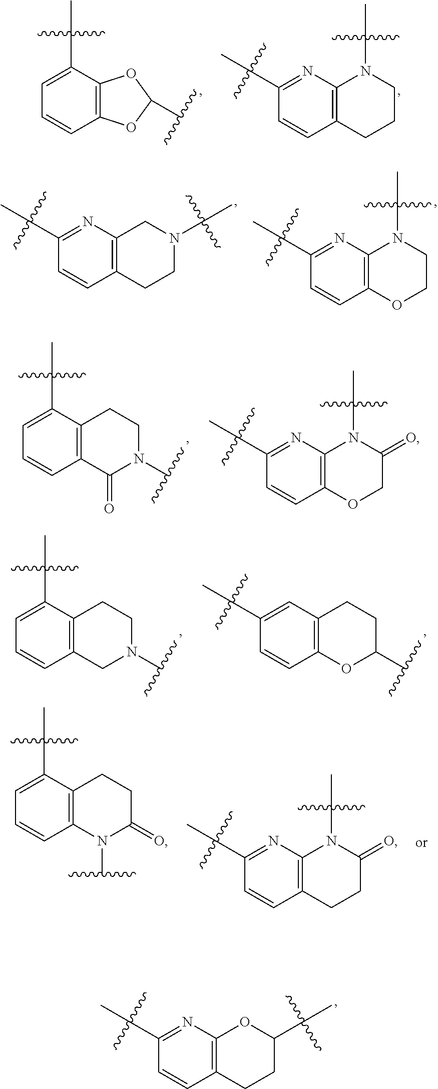
each of which is optionally substituted by halo, CN, C₃-C₆ cycloalkyl, or C₁-C₆ alkyl optionally substituted by halo or OH.

15. The compound of any of one of claims 1-12, or a pharmaceutically acceptable salt thereof, wherein Ring A is 5- to 12-membered heteroaryl, which is optionally substituted by halo, CN, C₃-C₆ cycloalkyl, or C₁-C₆ alkyl optionally substituted by halo or OH.

16. The compound of any one of claims 1-12 or 15, wherein Ring A is 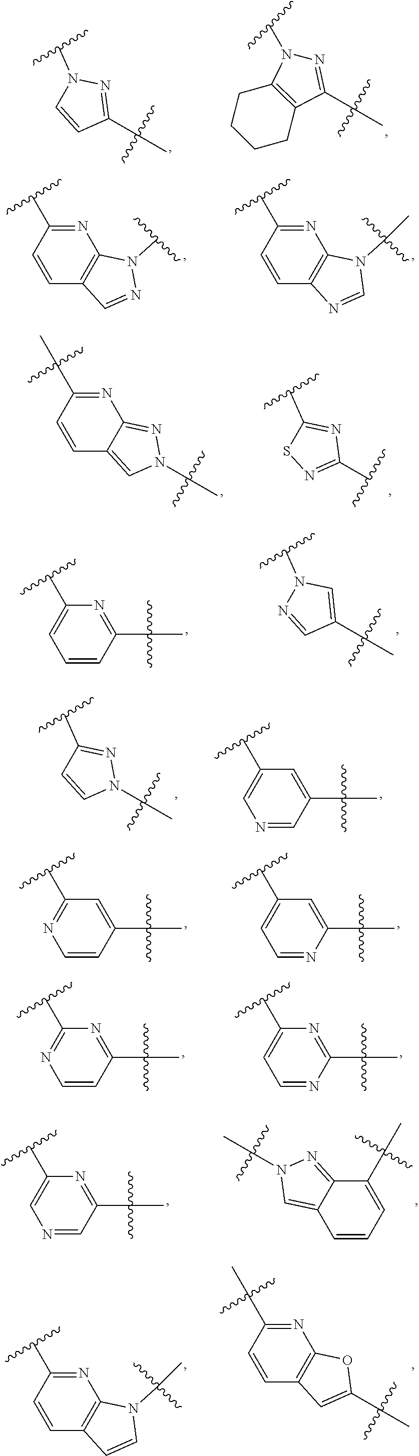 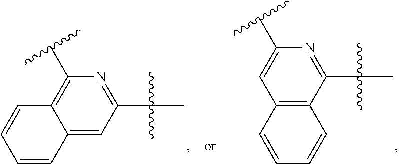 [SMILES: *c1cc2ccccc2c(*)n1 ; *c1cc2ccccc2c(*)n1]
each of which is independently optionally substituted by halo, CN, C₃-C₆ cycloalkyl, or C₁-C₆ alkyl optionally substituted by halo or OH.

17. The compound of any of one of claims 1-12 or 15, or a pharmaceutically acceptable salt thereof, wherein Ring A is 5- to 6-membered heteroaryl, which is optionally substituted by halo, CN, C₃-C₆ cycloalkyl, or C₁-C₆ alkyl optionally substituted by halo or OH.

18. The compound of any one of claims 1-12, 15, or 17, or a pharmaceutically acceptable salt thereof, wherein Ring A is 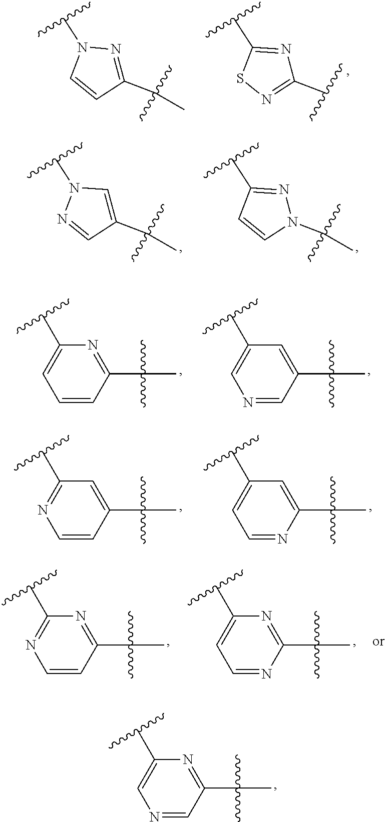
each of which is independently optionally substituted by halo, CN, C₃-C₆ cycloalkyl, or C₁-C₆ alkyl optionally substituted by halo or OH.

19. The compound of any one of claims 1-18, or a pharmaceutically acceptable salt thereof, wherein L is a bond.

20. The compound of any one of claims 1-18, or a pharmaceutically acceptable salt thereof, wherein L is —O—.

21. The compound of any one of claims 1-18, or a pharmaceutically acceptable salt thereof, wherein L is C₁-C₆ alkylene optionally substituted by R^{L1}.

22. The compound of claim 21, or a pharmaceutically acceptable salt thereof, wherein L is C₂ alkylene optionally substituted by R^{L1}.

23. The compound of any one of claims 1-18, or a pharmaceutically acceptable salt thereof, wherein L is *—O—C₁-C₆ alkylene-** optionally substituted by R^{L}.

24. The compound of any one of claims 1-18, or a pharmaceutically acceptable salt thereof, wherein L is *—C₁-C₆ alkylene-O—**.

25. The compound of any one of claims 1-18, or a pharmaceutically acceptable salt thereof, wherein L is *—NR⁶—C₁-C₆ alkylene-**.

26. The compound of any one of claims 1-25, or a pharmaceutically acceptable salt thereof, wherein Ring B is C₃-C₁₀ cycloalkyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl.

27. The compound of any one of claims 1-26, or a pharmaceutically acceptable salt thereof, wherein Ring B is 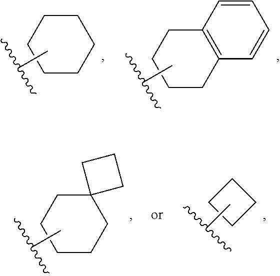
each of which is independently optionally substituted by one to three substituents independently selected from the group consisting of halo, CN, oxo, C₁-C₆ alkyl, —COCH₃, —CONH₂, —S(O)₂CH₃ and phenyl.

28. The compound of any one of claims 1-25, or a pharmaceutically acceptable salt thereof, wherein Ring B is C₆-C₁₄ aryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl.

29. The compound of any one of claims 1-25 or 28, or a pharmaceutically acceptable salt thereof, wherein Ring B is 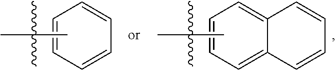
each of which is independently optionally substituted by one to three substituents each independently selected from the group consisting of halo, CN, oxo, C₁-C₆ alkyl, C₁-C₆ haloalkyl, —COCH₃, —CONH₂, —S(O)₂CH₃ and phenyl.

30. The compound of any one of claims 1-25 or 29, or a pharmaceutically acceptable salt thereof, wherein Ring B is phenyl substituted by one to three substituents independently selected from the group consisting of halo and CN.

31. The compound of any one of claims 1-25, or a pharmaceutically acceptable salt thereof, wherein Ring B is 4- to 12-membered heterocyclyl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl.

32. The compound of any one of claims 1-25 or 31, or a pharmaceutically acceptable salt thereof, wherein Ring B is 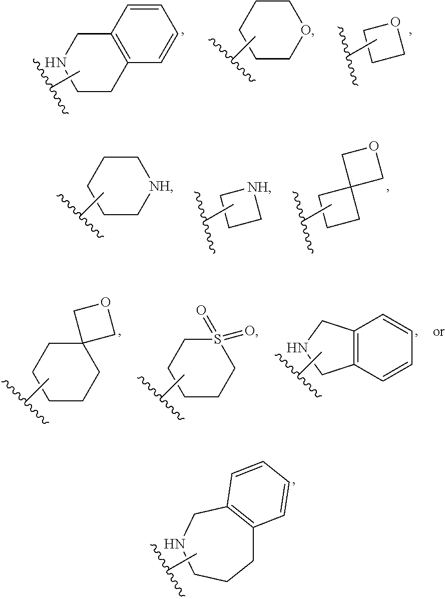
each of which is independently optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl.

33. The compound of any one of claims 1-25 or 31-32, or a pharmaceutically acceptable salt thereof, wherein Ring B is 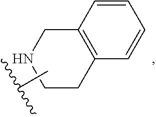
which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl.

34. The compound of any one of claims 1-25, or a pharmaceutically acceptable salt thereof, wherein Ring B is 5- to 12-membered heteroaryl, which is optionally substituted by one to three substituents independently selected from the group consisting of halo, CN, oxo, C₁-C₆ alkyl, C₁-C₆ haloalkyl, —COCH₃, —CONH₂, —S(O)₂CH₃ and phenyl.

35. The compound any one of claims 1-25 or 34, or a pharmaceutically acceptable salt thereof, wherein Ring B is 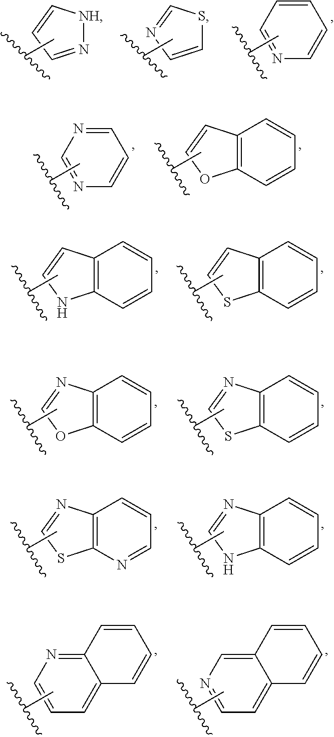
each of which is independently optionally substituted by one to three substituents independently selected from the group consisting of halo, CN, oxo, C₁-C₆ alkyl, —COCH₃, —CONH₂, —S(O)₂CH₃ and phenyl.

36. A compound of Formula (VI) 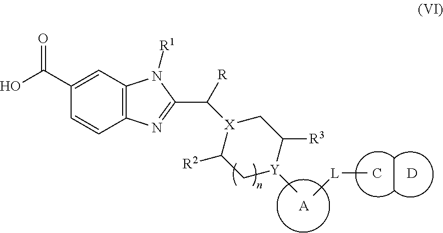
or a pharmaceutically acceptable salt thereof, wherein
X is N or CH;
n is 0 or 1;
R¹ is —C₁-C₆ alkylene-R⁵, wherein R⁵ is 3- to 6-membered heterocyclyl or 5- to 6-membered heteroaryl, each of which is independently optionally substituted by C₁-C₆ alkyl;
R² is hydrogen, oxo, or C₁-C₆ alkyl;
Ring A is 5- to 12-membered heterocyclyl or 5- to 12-membered heteroaryl, each of which is independently optionally substituted by halo, CN, C₃-C₆ cycloalkyl, or C₁-C₆ alkyl optionally substituted by halo or OH;
L is a bond, —O—, C₁-C₆ alkylene, *—O—C₁-C₆ alkylene-**, *—C₁-C₆ alkylene-O—**, or *—NR⁶—C₁-C₆ alkylene-**, wherein
* represents the point of attachment to ring A and ** represents the point of attachment to 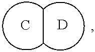
when L is *—O—C₁-C₆ alkylene-**, the C₁-C₆ alkylene is optionally substituted by R^{L}, wherein:
each R^{L} is independently C₁-C₆ alkyl or halo, or
two R^{L} are taken together with the carbon atom or atoms to which they are attached to form C₃-C₆ cycloalkyl or 3- to 6-membered heterocyclyl;
when L is C₁-C₆ alkylene, the C₁-C₆ alkylene is optionally substituted by R^{L1}, wherein:
each R^{L1} is independently halo, OH, or C₁-C₆ alkyl; or
two R^{L1} are taken together with the carbon atom or atoms to which they are attached to form C₃-C₆ cycloalkyl or 3- to 6-membered heterocyclyl, and
R⁶ is hydrogen or C₁-C₆ alkyl; and 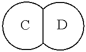
is a fused bicyclic ring system comprising fused rings Ring C and Ring D, wherein Ring C is C₅-C₆ cycloalkyl, 5- to 7-membered heterocyclyl, or 5- to 6-membered heteroaryl; and
Ring D is C₆ cycloalkyl, C₆ aryl or 6-membered heteroaryl;
wherein Ring C and Ring D are optionally substituted by one to three substituents independently selected from the group consisting of halo, CN, oxo, C₁-C₆ alkyl, C₁-C₆ haloalkyl, —COCH₃, —CONH₂, —S(O)₂CH₃, and phenyl.

37. The compound of claim 36, or a pharmaceutically acceptable salt thereof, wherein R¹ is —CH₂—R⁵.

38. The compound of claim 36 or 37, or a pharmaceutically acceptable salt thereof, wherein R⁵ is 3- to 6-membered heterocyclyl, which is optionally substituted by C₁-C₆ alkyl.

39. The compound of any one of claims 36-38, or a pharmaceutically acceptable salt thereof, wherein R⁵ is 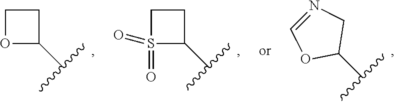 [SMILES: *C1CCO1 ; *C1CCS1(=O)=O ; *C1CN=CO1]
each of which is independently optionally substituted by C₁-C₆ alkyl.

40. The compound of any one of claims 36-39, or a pharmaceutically acceptable salt thereof, wherein R⁵ is

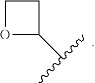 [SMILES: *C1CCO1]

41. The compound of claim 36 or 37, or a pharmaceutically acceptable salt thereof, wherein R⁵ is 5- to 6-membered heteroaryl, which is optionally substituted by C₁-C₆ alkyl.

42. The compound of any one of claims 36-37 or 41, or a pharmaceutically acceptable salt thereof, wherein R⁵ is 5-membered heteroaryl, which is optionally substituted by C₁-C₆ alkyl.

43. The compound of any one of claims 36-37 or 41-42, or a pharmaceutically acceptable salt thereof, wherein R⁵ is 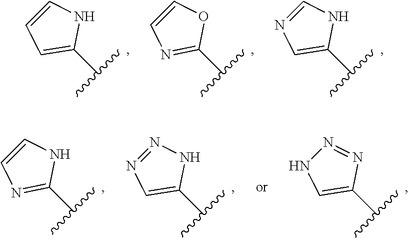 [SMILES: *c1ccc[nH]1 ; *c1ncco1 ; *c1cnc[nH]1 ; *c1ncc[nH]1 ; *c1cnn[nH]1 ; *c1c[nH]nn1]
each of which is optionally substituted by C₁-C₆ alkyl.

44. The compound of any one of claims 36-37 or 41-43, or a pharmaceutically acceptable salt thereof, wherein R⁵ is 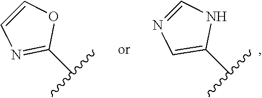 [SMILES: *c1ncco1 ; *c1cnc[nH]1]
each of which is optionally substituted by C₁-C₆ alkyl.

45. The compound of any one of claims 36-44, or a pharmaceutically acceptable salt thereof, wherein X is N.

46. The compound of any one of claims 36-45, or a pharmaceutically acceptable salt thereof, wherein n is 1.

47. The compound of any of one of claims 36-46, or a pharmaceutically acceptable salt thereof, wherein Ring A is 5- to 12-membered heterocyclyl, which is optionally substituted by halo, CN, C₃-C₆ cycloalkyl, or C₁-C₆ alkyl optionally substituted by halo or OH.

48. The compound of any one of claims 36-47, or a pharmaceutically acceptable salt thereof, wherein Ring A is Ring A is 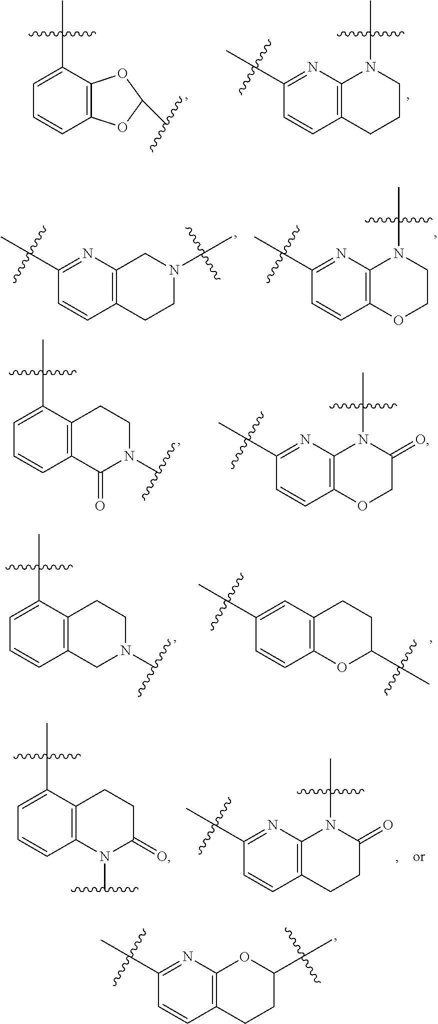
each of which is optionally substituted by halo, CN, C₃-C₆ cycloalkyl, or C₁-C₆ alkyl optionally substituted by halo or OH.

49. The compound of any of one of claims 36-46, or a pharmaceutically acceptable salt thereof, wherein Ring A is 5- to 12-membered heteroaryl, which is optionally substituted by halo, CN, C₃-C₆ cycloalkyl, or C₁-C₆ alkyl optionally substituted by halo or OH.

50. The compound of any one of claims 36-46 or 49, wherein Ring A is 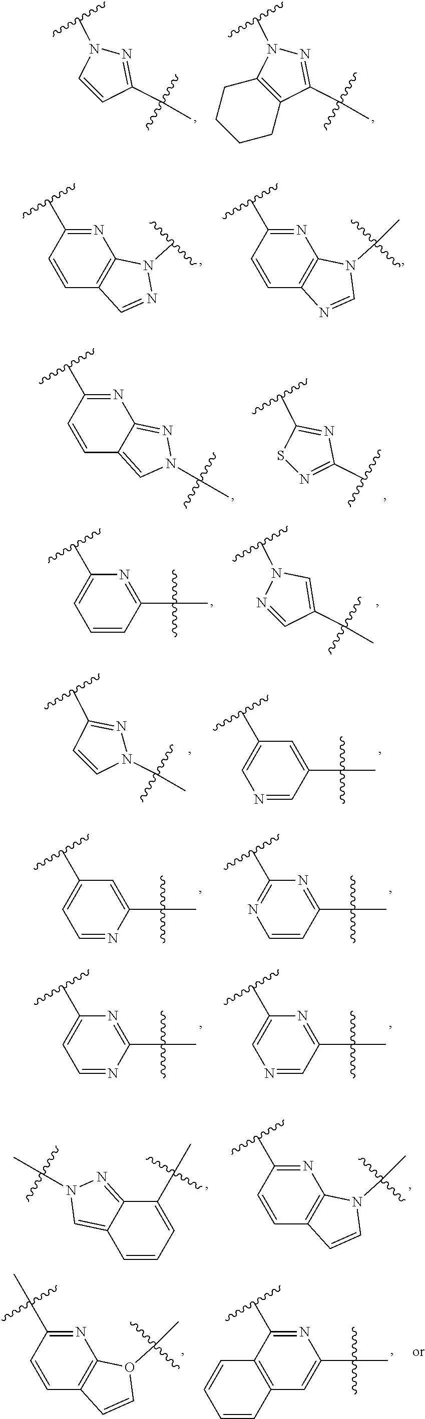 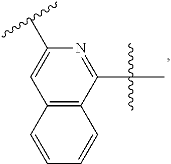 [SMILES: *c1cc2ccccc2c(*)n1]
each of which is independently optionally substituted by halo, CN, C₃-C₆ cycloalkyl, or C₁-C₆ alkyl optionally substituted by halo or OH.

51. The compound of any of one of claims 36-46 or 49, or a pharmaceutically acceptable salt thereof, wherein Ring A is 5- to 6-membered heteroaryl, which is optionally substituted by halo, CN, C₃-C₆ cycloalkyl, or C₁-C₆ alkyl optionally substituted by halo or OH.

52. The compound of any one of claims 36-46, 49, or 51, or a pharmaceutically acceptable salt thereof, wherein Ring A is 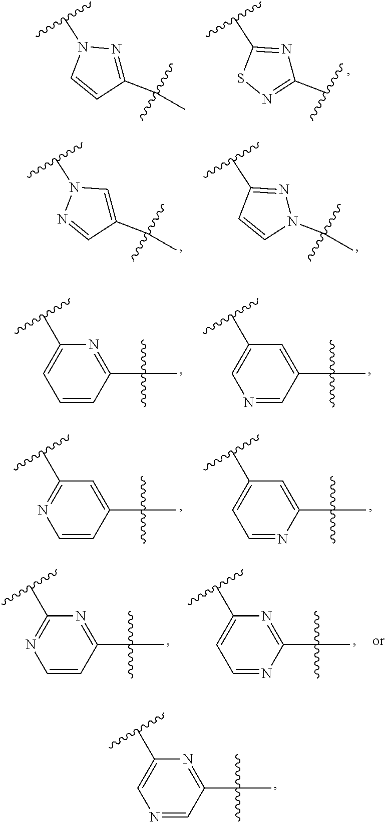
each of which is independently optionally substituted by halo, CN, C₃-C₆ cycloalkyl, or C₁-C₆ alkyl optionally substituted by halo or OH.

53. The compound of any one of claims 36-52, or a pharmaceutically acceptable salt thereof, wherein L is a bond.

54. The compound of any one of claims 36-52, or a pharmaceutically acceptable salt thereof, wherein L is —O—.

55. The compound of any one of claims 36-52, or a pharmaceutically acceptable salt thereof, wherein L is C₁-C₆ alkylene optionally substituted by R^{L1}.

56. The compound of claim 55, or a pharmaceutically acceptable salt thereof, wherein L is C₂ alkylene optionally substituted by R^{L1}.

57. The compound of any one of claims 36-52, or a pharmaceutically acceptable salt thereof, wherein L is *—O—C₁-C₆ alkylene-** optionally substituted by R^{L}.

58. The compound of any one of claims 36-52, or a pharmaceutically acceptable salt thereof, wherein L is *—C₁-C₆ alkylene-O—**.

59. The compound of any one of claims 36-52, or a pharmaceutically acceptable salt thereof, wherein L is *—NR⁶—C₁-C₆ alkylene-**.

60. The compound of any one of claims 36-59, or a pharmaceutically acceptable salt thereof, wherein Ring D is C₆ aryl.

61. The compound of claim 60, wherein Ring C is C₅-C₆ cycloalkyl.

62. The compound of claim 60, wherein Ring C is 5- to 7-membered heterocyclyl.

63. The compound of claim 60, wherein Ring C is 5- to 6-membered heteroaryl.

64. The compound of any one of claims 36-59, or a pharmaceutically acceptable salt thereof, wherein Ring D is 6-membered heteroaryl.

65. The compound of claim 64, wherein Ring C is C₅-C₆ cycloalkyl.

66. The compound of claim 64, wherein Ring C is 5- to 7-membered heterocyclyl.

67. The compound of claim 64, wherein Ring C is 5- to 6-membered heteroaryl.

68. A compound or a pharmaceutically acceptable salt thereof, wherein the compound is selected from the group consisting of the compounds in Table 1.

69. A pharmaceutical composition comprising the compound of any one of claims 1-68, or a pharmaceutically acceptable salt thereof, and a pharmaceutical acceptable excipient.

70. A method of treating a disease mediated by glucagon-like peptide-1 receptor (GLP-1R) in an individual in need thereof, comprising administering to the individual a therapeutically effective amount of the compound of any one of claims 1-68, or a pharmaceutically acceptable salt thereof, or the pharmaceutical composition of claim 69.

71. The method of claim 70, wherein the disease is a liver disease.

72. The method of claim 71, wherein the liver disease is primary biliary cirrhosis (PBC), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, nonalcoholic fatty liver disease (NAFLD), nonalcoholic steatohepatitis (NASH), graft versus host disease, transplant liver regeneration, congenital hepatic fibrosis, choledocholithiasis, granulomatous liver disease, intra- or extrahepatic malignancy, Sjogren's syndrome, sarcoidosis, Wilson's disease, Gaucher's disease, hemochromatosis, or oti-antitrypsin deficiency.

73. The method of claim 70, wherein the disease is diabetes.

74. The method of claim 70, wherein the disease is a cardiometabolic disease.

75. The method of claim 70, wherein the disease is obesity.

76. Use of the compound of any one of claims 1-68, or a pharmaceutically acceptable salt thereof, in the manufacture of a medicament for treating a disease mediated by mediated by GLP-1R.
